(19) United States
(12) Patent Application Publication
(10) Pub. No.: US 2023/0203087 A1
(43) Pub. Date: Jun. 29, 2023

(54) OLIGONUCLEOTIDE COMPOSITIONS AND METHODS THEREOF

(71) Applicants: Pachamuthu Kandasamy, Cambridge, MA (US); Jayakanthan Kumarasamy, Cambridge, MA (US); Chandra Vargeese, Cambridge, MA (US); Subramanian Marappan, Cambridge, MA (US); Gopal Bommineni, Cambridge, MA (US); Mamoru Shimizu, Cambridge, MA (US); Naoki Iwamoto, Cambridge, MA (US); Stephany Standley, Cambridge, MA (US); Yuanjing Liu, Cambridge, MA (US); Amy Jada Andreucci, Cambridge, MA (US); Genliang LU, Cambridge, MA (US); Onanong Chivatakarn, Cambridge, MA (US); Akbar Husain Khan, Cambridge, MA (US); WAVE LIFE SCIENCES LTD., Singapore (SG)

(72) Inventors: Pachamuthu Kandasamy, Lexington, MA (US); Jayakanthan Kumarasamy, Arlington, MA (US); Chandra Vargeese, Schwenksville, PA (US); Subramanian Marappan, Acton, MA (US); Gopal Reddy Bommineni, Belmont, MA (US); Mamoru Shimizu, Arlington, MA (US); Naoki Iwamoto, Boston, MA (US); Stephany Michelle Standley, Wakefield, MA (US); Yuanjing Liu, Boston, MA (US); Amy Jada Andreucci, Belmont, MA (US); Genliang Lu, Winchester, MA (US); Onanong Chivatakarn, Auburndale, MA (US); Akbar Husain Khan, Shrewsbury, MA (US)

(21) Appl. No.: 17/926,987

(22) PCT Filed: May 24, 2021
(86) PCT No.: PCT/US21/33945

§ 371 Date: Nov. 21, 2022

Related U.S. Application Data
(60) Provisional application No. 63/029,387, filed on May 22, 2020.

(51) Int. Cl. C07H 21/04; C07F 9/165; C07D 295/02

(52) U.S. Cl. CPC C07H 21/04; C07F 9/165; C07D 295/02

(57) ABSTRACT

The present disclosure provides modified oligonucleotides and compositions and methods thereof. In some embodiments, provided technologies comprise modified sugars and/or modified internucleotidic linkages. In some embodiments, the present disclosure provides technologies for preparing modified oligonucleotides. In some embodiments, the present disclosure provides chirally controlled oligonucleotide compositions and methods for their preparation and uses.

### CROSS-REFERENCE TO RELATED APPLICATION

This application claims priority to U.S. Provisional Application No. 63/029,387, filed May 22, 2020, the entirety of which is incorporated herein by reference.

### BACKGROUND

Oligonucleotides are useful in various applications, e.g., therapeutic, diagnostic, and/or research applications. For example, oligonucleotides targeting various genes can be useful for treatment of conditions, disorders or diseases related to such target genes.

### SUMMARY

Oligonucleotides are useful for many purposes. However, natural oligonucleotides have been found to suffer disadvantages, such as low stability, low activity, etc., that can reduce or negate their usefulness, e.g., as therapeutics. Certain technologies have been developed that can improve oligonucleotide properties and usefulness. For example, certain modifications, e.g., to nucleobases, sugars, and/or internucleotidic linkages, etc., have been described that can improve oligonucleotide properties and/or activities. In some embodiments, technologies that permit chiral control of chiral internucleotidic linkages can be particularly useful and effective.

Among other things, the present Applicant appreciated that technologies that can effectively incorporate various type of modifications and/or patterns thereof (e.g., those described in various embodiments of present disclosure), particularly into chirally controlled oligonucleotide compositions, can provide significant benefits and advantages. In various embodiments, the present disclosure describes developments of oligonucleotides and compositions thereof, particularly chirally controlled oligonucleotide compositions, that can provide various benefits and advantages (e.g., with respect to stability, activity, delivery, selectivity, clearance, toxicity, etc.), and may be particularly useful, for example, for therapeutic uses.

For example, in some embodiments, the present disclosure provides oligonucleotides comprising one or more modified sugars which are connected to internucleotidic linkages through nitrogen atoms (e.g., morpholine as in various oligonucleotides described herein). In some embodiments, provided oligonucleotides comprise one or more acyclic sugars. In some embodiments, provided oligonucleotides comprises one or more one or more modified sugars which are connected to internucleotidic linkages through nitrogen atoms or one or more acyclic sugars, and one or more ribose sugars each of which is independently and optionally modified. In some embodiments, provided oligonucleotides comprises one or more one or more modified sugars which are connected to internucleotidic linkages through nitrogen atoms or one or more acyclic sugars, and one or more ribose sugars each of which is independently and optionally modified. In some embodiments, provided oligonucleotides comprises one or more one or more modified sugars which are connected to internucleotidic linkages through nitrogen atoms or one or more acyclic sugars, and one or more modified ribose sugars (different from sugars typically found in natural DNA and RNA molecules, e.g., those with R^{2s} that are not —H or —OH). In some embodiments, provided oligonucleotides comprises one or more one or more modified sugars which are connected to internucleotidic linkages through nitrogen atoms or one or more acyclic sugars, one or more modified ribose sugars, and one or more natural DNA sugars (which, as appreciated by those skilled in the art, have no substitution at 2′-carbon as typically found in natural DNA molecules).

As demonstrated in many embodiments herein, the present disclosure provides oligonucleotides comprising sugars, including modified sugars described above, connected by various types of internucleotidic linkages, e.g., natural phosphate linkages (as typically found in natural DNA and RNA molecules), modified internucleotidic linkages comprising linkage phosphorus, modified internucleotidic linkages that comprise no linkage phosphorus (e.g., —C(O)—O— or —C(O)—N(R′)— as described in various embodiments, in which, in some embodiments, —C(O)— may be bonded to a nitrogen atom of a sugar, and —O— or —N(R′)— may be bonded to a carbon atom of a sugar). In some embodiments, modified internucleotidic linkages comprising linkage phosphorus are non-negatively charged internucleotidic linkages; in some embodiments, they are neutral internucleotidic linkages. In some embodiments, modified internucleotidic linkages comprise nitrogen atoms bonded to linkage phosphorus atoms, wherein the nitrogen atoms are not bonded to sugar atoms (e.g., sugar carbon atoms). In some embodiments, provided technologies provide chiral control of chiral internucleotidic linkages, e.g., control of stereochemical configurations of chiral linkage phosphorus atoms.

In some embodiments, provided technologies comprise one or more modified sugars (e.g., those described above) and/or one or more modified internucleotidic linkages (e.g., those described above), wherein one or more chiral internucleotidic linkages are independently chirally controlled. Among other things, the present disclosure provides technologies that are particularly useful for chirally controlled compositions of such oligonucleotides. For example, in some embodiments, the present disclosure provides technologies that are particularly effective for incorporating certain types of sugars (e.g., those bonded to linkage phosphorus through nitrogen atoms) which are compatible with chirally controlled incorporation of various types of internucleotidic linkages, e.g., various internucleotidic linkages having the structure of —Y—P^{L}(—X—R^{L})—Z— such as natural phosphate linkages, n006, etc. In some embodiments, each linkage having the structure of —Y—P^{L}(—X—R^{L})—Z— is independently chirally controlled. In some embodiments, each phosphorothioate internucleotidic linkage is independently chirally controlled.

In some embodiments, an oligonucleotides of the present disclosure, e.g., in an oligonucleotide composition such as a chirally controlled oligonucleotide composition (e.g., an oligonucleotide of a plurality in chirally controlled oligonucleotide compositions) comprises:

a sugar that is bonded to an internucleotidic linkage through a nitrogen atom and/or an acyclic sugar, and/or an internucleotidic linkage having the structure of:

—Y—P^{L}(—X—R^{L})—Z—,

—C(O)—O—,

—C(O)—N(R′)—, or

-L^{L1}-Cy^{IL}-L^{L2}-;

wherein:

P^{L} is P, P(═W), P->B(-L^{L}-R^{L})₃, or P^{N};

W is O, N(-L^{L}-R^{L}), S or Se;

each of X, Y and Z is independently —O—, —S—, —N(-L^{L}-R^{L})—, or L^{L}-;

each R^{L} is independently -L^{L}-R′ or —N═C(-L^{L}-R′)₂;

P^{N} is P═N—C(-L^{L}-R′)(=L^{N}-R′) or P═N-L^{L}-R^{L};

L^{N} is ═N-L^{L1}-, ═CH-L^{L1}—wherein CH is optionally substituted, or ═N⁺(R′)(Q⁻)-L^{L1}-;

Q⁻ is an anion;

each of L^{s}, L^{L1}, L^{L2} and L^{L} is independently L;

-Cy^{IL}- is -Cy-;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)—, —OP(SR′)—, —OP(NR′)—, —OP(R′)—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R; each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, P^{L} is P, P(═W), or P^{N}. In some embodiments, P^{L} is P. In some embodiments, P^{L} is P(═O). In some embodiments, P^{L} is P(═S). In some embodiments, P^{L} is P^{N}. In some embodiments, a linkage has the structure of —Y—P^{L}(—X—R^{L})—Z—, or a salt form thereof.

In some embodiments, an oligonucleotide comprises a sugar that is bonded to an internucleotidic linkage through a nitrogen atom. In some embodiments, an oligonucleotide comprises a sugar that is bonded to an internucleotidic linkage through a nitrogen atom, and an internucleotidic linkage having the structure of —P^{L}(—X—R^{L})—Z—, —C(O)—O—, or —C(O)—N(R′)—, wherein the P^{L} or —C(O)— is bonded to the nitrogen of the sugar. In some embodiments, a sugar has the structure of

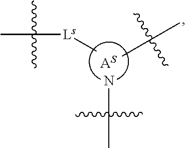

wherein Ring As is an optionally substituted 3-30 membered, monocyclic, bicyclic or polycyclic ring having, in addition to the nitrogen, 0-10 heteroatoms, and L^{s} is Las described herein. In some embodiments, an oligonucleotide comprises a sugar having the structure of

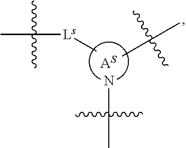

and an internucleotidic linkage having the structure of —P^{L}(—X—R^{L})—Z—, —C(O)—O—, —C(O)—N(R′)—, or -L^{L1}-Cy^{IL}-L^{L2}-, wherein each variable is independently as described herein. In some embodiments, an oligonucleotide comprises a sugar having the structure of

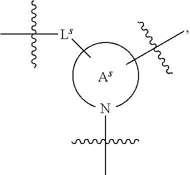

and an internucleotidic linkage having the structure of —P^{L}(—X—R^{L})—Z—, —C(O)—O—, or —C(O)—N(R′)—, wherein each variable is independently as described herein. In some embodiments, an oligonucleotide comprises a sugar having the structure of

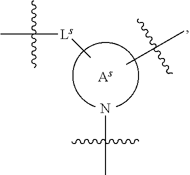

and an internucleotidic linkage having the structure of —P^{L}(—X—R^{L})—Z—, —C(O)—O—, or —C(O)—N(R′)—, wherein the P^{L} or —C(O)— is bonded to the nitrogen of the sugar, each variable is independently as described herein.

In some embodiments, an oligonucleotide comprises an acyclic sugar. In some embodiments, an acyclic sugar has the structure of —CH₂; —CH(-L^{SA}-)-CH₂—, wherein each of the CH₂ and CH is independently optionally substituted, and L^{SA} is L as described herein. In some embodiments, L^{SA} is —O—CH₂—, wherein the —CH₂;—is optionally substituted. In some embodiments, L^{SA} is bonded to a nucleobase. In some embodiments, each of the optionally substituted —CH₂;—is independently bonded to an internucleotidic linkage. In some embodiments, an acyclic sugar is —CH₂; —CH(—O—CH₂; —)—CH₂—, —CH₂; —CH(—O—CH₂)—CH(CH₃)—, —CH₂; —CH(—O—CH(CH₃)—)—CH₂—, or —CH₂; —CH(—O—CH(CH₂OH)—)—CH₂—. In some embodiments,

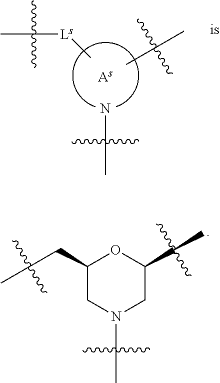

In some embodiments, an oligonucleotide comprises an internucleotidic linkage having the structure of —Y—P^{L}(—X—R^{L})—Z—. In some embodiments, Y is a covalent bond. In some embodiments, an oligonucleotide comprises an internucleotidic linkage having the structure of —P^{L}(—X—R^{L})—Z—. In some embodiments, P^{L} is bonded to a sugar through a nitrogen atom. In some embodiments, P^{L} is P. In some embodiments, P^{L} is P(═W). In some embodiments, P^{L} is P(═O). In some embodiments, P^{L} is P^{N}. In some embodiments, Z is —O—. In some embodiments, Y is —O— and Z is —O—. In some embodiments, an comprises an internucleotidic linkage having the structure of —C(O)-O— or —C(O)—N(R′)—. In some embodiments, an comprises an internucleotidic linkage having the structure of —C(O)-—O— or —C(O)—N(R′)—, wherein —C(O)— is bonded to a sugar through a nitrogen atom. In some embodiments, —O— or —N(R′)— is bonded a carbon atom of a sugar. In some embodiments, an oligonucleotide comprises an internucleotidic linkage having the structure of -L^{L1}-Cy^{IL}-L^{L2}-. In some embodiments, each of L^{L1} and L^{L2} is independently optionally substituted bivalent C₁₋₆ aliphatic or heteroaliphatic having 1-4 heteroatoms. In some embodiments, each of L^{L1} and L^{L2} is independently optionally substituted bivalent C₁₋₆ aliphatic. In some embodiments, -Cy^{IL}—is independently an optionally substituted 5-6 membered heteroaryl ring having 1-4 heteroatoms. In some embodiments, -Cy^{IL}—is independently an optionally substituted 5-6 membered heteroaryl ring having 1-4 heteroatoms. In some embodiments, -Cy^{IL}—is

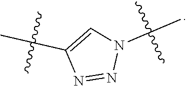 [SMILES: *c1cn(*)nn1]

In some embodiments, the present disclosure provides oligonucleotide compositions, particularly chirally controlled oligonucleotide compositions in which configurations of one or more or all linkage phosphorus are each independently chirally controlled. In some embodiments, the present disclosure provides an oligonucleotide composition comprising a plurality of oligonucleotides, wherein oligonucleotides of the plurality share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein the composition is enriched, relative to a substantially racemic preparation of oligonucleotides having the same common base sequence, for oligonucleotides of the plurality.

In some embodiments, the present disclosure provides an oligonucleotide composition comprising a plurality of oligonucleotides, wherein oligonucleotides of the plurality share:

1) a common constitution, and

2) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),

wherein the composition is enriched, relative to a substantially racemic preparation of oligonucleotides sharing the common constitution, for oligonucleotides of the plurality.

As appreciated by those skilled in the art, oligonucleotides may be in various forms, e.g., acid forms, salt forms, etc. Unless indicated otherwise, references to oligonucleotides include various forms of such oligonucleotides. In some embodiments, the present disclosure provides pharmaceutical compositions comprising a provided oligonucleotide and a pharmaceutically acceptable carrier. In some embodiments, an oligonucleotide is in a salt form, e.g., pharmaceutically acceptable salt form. In some embodiments, a salt is a sodium salt.

Among other things, the present disclosure provides technologies (e.g., compounds, methods, etc.) useful for preparing oligonucleotides and compositions, particularly chirally controlled oligonucleotide compositions, of the present disclosure. In some embodiments, a provided method utilizes a compound of LG-I, LG-II, M-I, or M-II, or a salt thereof.

Technologies of the present disclosure are useful for various purposes. In some embodiments, provided technologies are useful for modulating levels of nucleic acids (e.g., transcripts, mRNA, etc.) and/or products thereof (e.g., proteins) in various systems (e.g., in vitro assays, cells, tissues, organs, organisms, subjects, etc.). In some embodiments, provided technologies can be utilized to reduce expression, levels, activities, etc. of target nucleic acids (e.g., transcripts, mRNA, etc.) and/or products thereof (e.g., through cleavage by RNase H, RNAi, etc., steric hindrance, etc.). In some embodiments, provided technologies can increase expression, levels, activities, etc. of target nucleic acids (e.g., transcripts, mRNA, etc.) and/or products thereof through modulation of splicing. Those skilled in the art appreciates that in many embodiments, base sequences of provided oligonucleotides may be complementary or identical to those of their target nucleic acids, and provided oligonucleotides may hybridize with target nucleic acids under suitable conditions.

### BRIEF DESCRIPTION OF THE DRAWINGS

FIG. 1. Provided technologies provide high activities. FIG. 1 demonstrates that provided technologies can provide effective splicing modulation provide desired exon-skipping products. H2K cells were grown in 96 well plates for 4 days, dosed, and left to further differentiate for an additional 4 days. RNA isolation was performed using the bead-based assay. cDNA was synthesized, pre-amplified, and multiplex Taqman was performed. Gblocks were used for quantification.

FIG. 2. Provided technologies provide high activities. FIG. 2 demonstrates that provided technologies can provide effective splicing modulation provide desired exon-skipping products. H2K cells were seeded into a 24WP (40K/W, pre-diff) and dosed (3-1-0.3 uM) for 3 h, washed, and further differentiated for 4 days prior to RNA extraction using Trizol and the Promega 96WP RNA kit. qPCR was performed on cDNA and % skipping values were extrapolated from an absolute curve generated with gBlocks.

FIG. 3. Provided technologies provide high activities. As demonstrated in FIG. 3, provided technologies can effectively reduce target nucleic acids. K562 cells were seeded into a 96WP (15K/W) and dosed (10 nM-3 uM) for 4 days prior to RNA extraction using the Promega 96WP kit. qPCR was performed on cDNA and %mRNA remaining values were normalized against mock values.

FIG. 4. Provided technologies provide high activities. As demonstrated in FIG. 4, provided technologies can effectively reduce target nucleic acids. GABA iNeurons with 4 day treatment.

FIG. 5. Provided technologies provide high activities. FIG. 5 demonstrates that provided technologies can provide effective splicing modulation provide desired exon-skipping products. H2K cells 4 days treatment.

FIG. 6. Provided oligonucleotide compositions provide activities in vivo. (A): Dosing schedule. (B): Provided compositions reduced mRNA levels. (C): Oligonucleotides were delivered to tissue.

FIG. 7. Technologies of the present disclosure can provide various advantages. (A): Schematic representation of dosing regimen with arrows indicating administration of intracerebroventricular dose (day 0, D0) and day of analysis (day 7, D7). Relative Malatl expression (normalized to Hprtl) in spinal cord (left) and cortex (right) one-week post treatment with PBS, WV-8587 and WV-11533 at the indicated dose. Data are presented as box and whisker plots with box from min to max with data from individual mice shown, n=8 * P<0.05, ** P<0.01, *** P<0.001 mixed-effects model with multiple comparisons. (B): Schematic representation of dosing regimen with arrow indicating administration of ICV dose (day 0, D0) and day of analysis (day 28, D28). Relative Malatl expression (normalized to Hprtl) in spinal cord (top left) and cortex (top right) 4-weeks post treatment with PBS, WV-8587 and WV-11533. Concentration of oligonucleotide detected in spinal cord (bottom left) and cortex (bottom right) 4-weeks post treatment with PBS, WV-8587 and WV-11533. Data are presented as in panel A, n=4 **** P<0.0001 one-way ANOVA with multiple comparisons. (C): Schematic representation of dosing regimen with arrow indicating administration of intracerebroventricular dose (day 0, D0) and day of analysis (day 70, D70). Relative Malatl expression (normalized to Hprtl) in indicated tissue in CNS 10-weeks post treatment with PBS, WV-8587 and WV-11533. Data are presented as in panel A, n=3 one-way ANOVA with multiple comparisons. Bottom row asterisks show comparison of WV-11533 to PBS; top row asterisks WV-11533 to WV-8587.

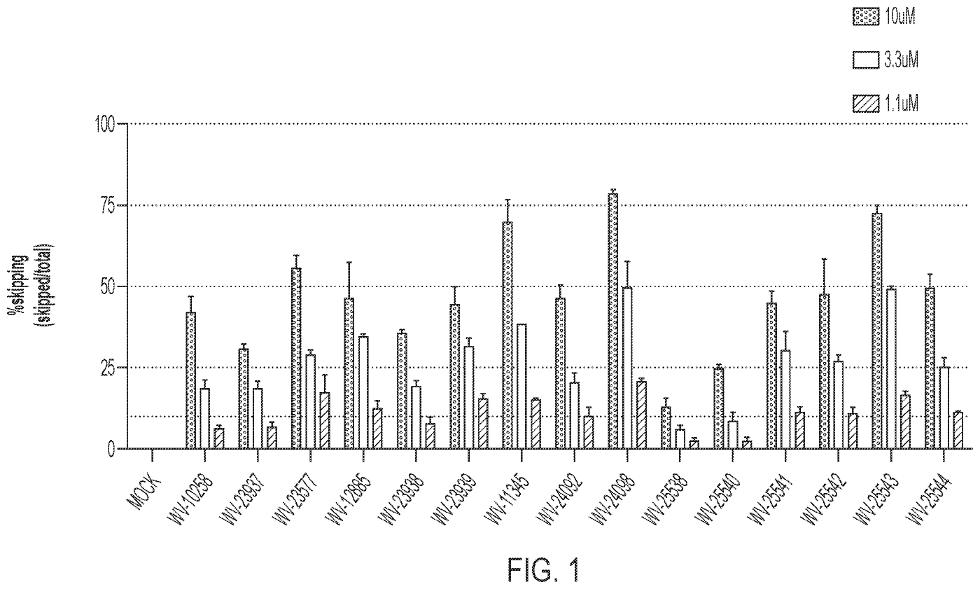

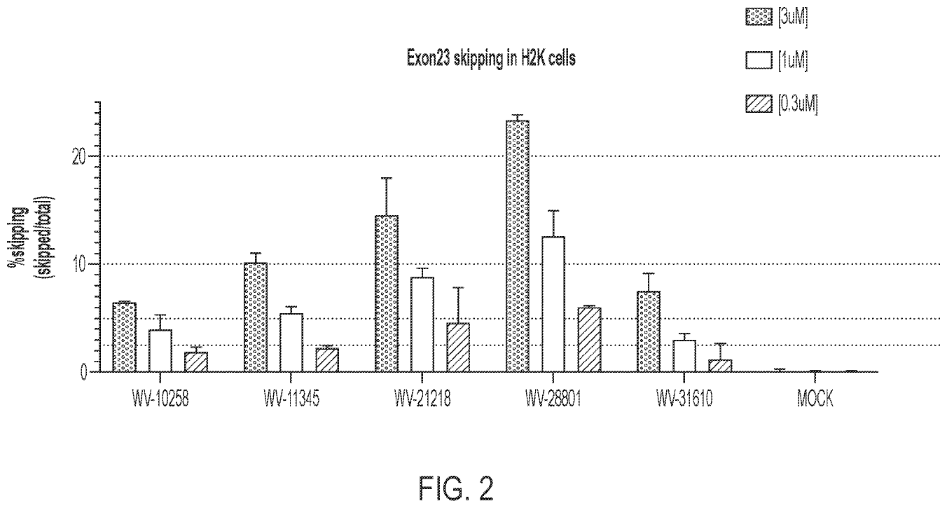

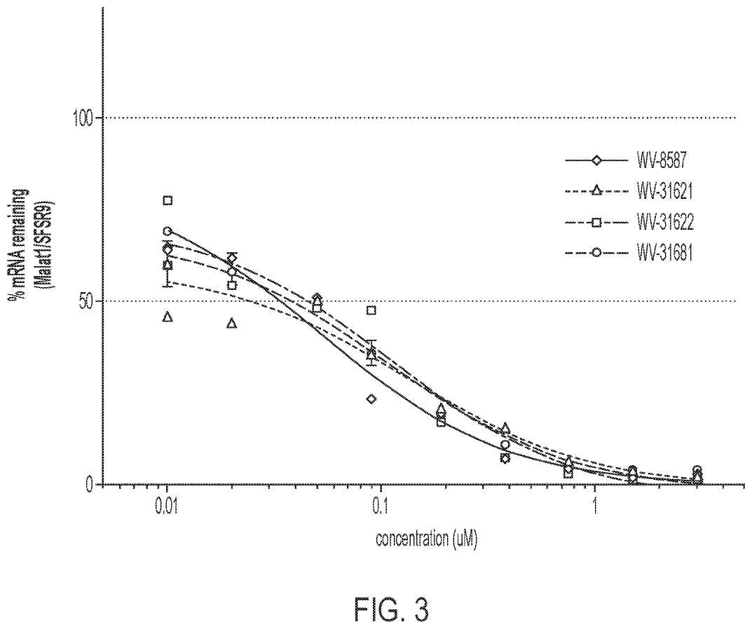

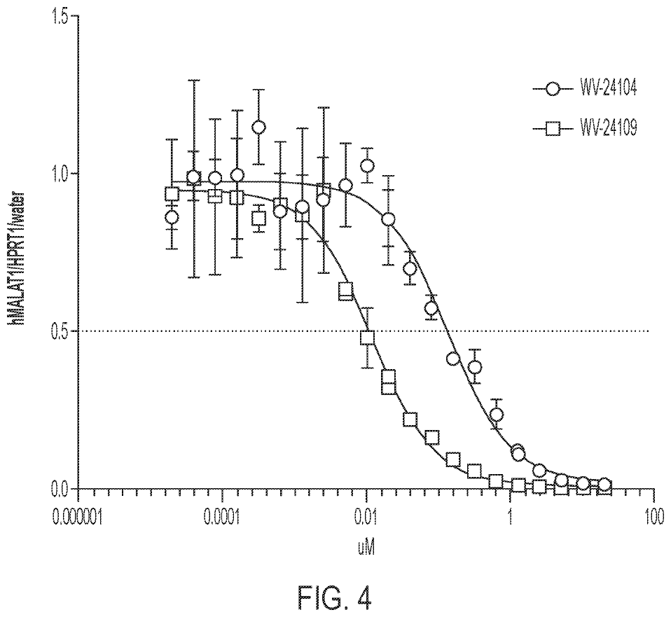

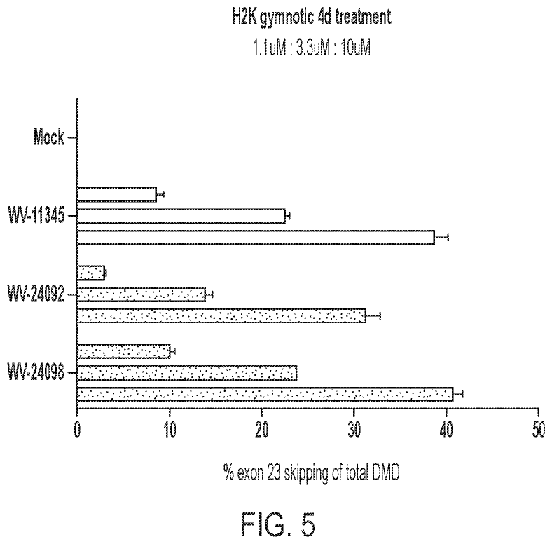

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, etc.) is from 5′ to 3′. Unless otherwise specified, oligonucleotides described herein may be provided and/or utilized in salt forms, particularly pharmaceutically acceptable salt forms, e.g., sodium salts. As those skilled in the art will appreciate, in some embodiments, individual oligonucleotides within a composition may be considered to be of the same constitution and/or structure even though, within such composition (e.g., a liquid composition), particular such oligonucleotides might be in different form(s) (e.g., different salt form(s) and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with Ft) are of the same constitution and/or structure, such individual oligonucleotides may properly be considered to be of the same constitution and/or structure (and share the same pattern of backbone linkages and/or pattern of backbone chiral centers).

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Antisense: The term “antisense”, as used herein, refers to a characteristic of an oligonucleotide or other nucleic acid having a base sequence complementary or substantially complementary to a target nucleic acid to which it is capable of hybridizing. In some embodiments, a target nucleic acid is a target gene mRNA. In some embodiments, hybridization is required for or results in at one activity, e.g., an increase in the level of skipping of a deleterious exon in a target nucleic acid and/or an increase in production of a gene product produced from a target nucleic acid from which a deleterious exon has been skipped. The term “antisense oligonucleotide”, as used herein, refers to an oligonucleotide complementary to a target nucleic acid. In some embodiments, an antisense oligonucleotide is capable of directing an increase in the level of skipping of a deleterious exon in a target nucleic acid and/or increase in production of a gene product produced from a target nucleic acid from which a deleterious exon has been skipped.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non—aromatic rings, such as indanyl, phthalimidyl, naphthimidyl, phenanthridinyl, or tetrahydronaphthyl, and the like.

Chiral control: As used herein, “chiral control” refers to control of the stereochemical designation of the chiral linkage phosphorus in a chiral internucleotidic linkage within an oligonucleotide. As used herein, a chiral internucleotidic linkage is an internucleotidic linkage whose linkage phosphorus is chiral. In some embodiments, a control is achieved through a chiral element that is absent from the sugar and base moieties of an oligonucleotide, for example, in some embodiments, a control is achieved through use of one or more chiral auxiliaries during oligonucleotide preparation as described in the present disclosure, which chiral auxiliaries often are part of chiral coupling partners (e.g., chiral phosphoramidites) used during oligonucleotide preparation. In contrast to chiral control, a person having ordinary skill in the art appreciates that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in each chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share a common constitution; or which share 1) a common base sequence, and/or 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled (e.g., through chirally controlled oligonucleotide preparation to stereoselectively form one or more chiral internucleotidic linkages). In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, oligonucleotides (or nucleic acids) of a plurality are of the same constitution. In some embodiments, level of the oligonucleotides (or nucleic acids) of the plurality is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides (or nucleic acids) in a composition that share the same constitution as the oligonucleotides (or nucleic acids) ofthe plurality. In some embodiments, each chiral internucleotidic linkage is a chiral controlled internucleotidic linkage, and the composition is a completely chirally controlled oligonucleotide composition. In some embodiments, oligonucleotides (or nucleic acids) of a plurality are structurally identical. In some embodiments, a chirally controlled internucleotidic linkage has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, a chirally controlled internucleotidic linkage has a diastereopurity of at least 95%. In some embodiments, a chirally controlled internucleotidic linkage has a diastereopurity of at least 96%. In some embodiments, a chirally controlled internucleotidic linkage has a diastereopurity of at least 97%. In some embodiments, a chirally controlled internucleotidic linkage has a diastereopurity of at least 98%. In some embodiments, a chirally controlled internucleotidic linkage has a diastereopurity of at least 99%. In some embodiments, a percentage of a level is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, a percentage of a level is or is at least (DS)^{nc}, wherein DS is 95%-100%. For example, when DS is 99% and nc is 10, the percentage is or is at least 90% ((99%)¹⁰≈0.90=90%). In some embodiments, level of a plurality of oligonucleotides in a composition is represented as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy). In some embodiments, not all chiral internucleotidic linkages are chiral controlled internucleotidic linkages, and the composition is a partially chirally controlled oligonucleotide composition. In some embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramidite method). In some embodiments, oligonucleotides (or nucleic acids) of a plurality are of the same type. In some embodiments, a chirally controlled oligonucleotide composition comprises non-random or controlled levels of individual oligonucleotide or nucleic acids types. For instance, in some embodiments a chirally controlled oligonucleotide composition comprises one and no more than one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of an oligonucleotide type, which composition comprises a non-random or controlled level of a plurality of oligonucleotides of the oligonucleotide type.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)—, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H—quinolizinyl, carbazolyl, acridinyl, phenazinyl, phenothiazinyl, phenoxazinyl, tetrahydroquinolinyl, tetrahydroisoquinolinyl, and pyrido [2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is oxygen, sulfur or nitrogen. In some embodiments, at least one heteroatom is nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Identity: As used herein, the term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using a NWSgapdna.CMP matrix.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In some embodiments, an internucleotidic linkage is a modified internucleotidic linkage (not a natural phosphate linkage). In some embodiments, an internucleotidic linkage is a “modified internucleotidic linkage” wherein at least one oxygen atom or —OH of a phosphodiester linkage is replaced by a different organic or inorganic moiety. In some embodiments, such an organic or inorganic moiety is selected from =S, =Se, =NR′, —SR′, —SeR′, —N(R′)₂, B(R′)₃, —S—, —Se—, and —N(R′)—, wherein each R′ is independently as defined and described in the present disclosure. In some embodiments, an internucleotidic linkage is a phosphorothioate linkage (or phosphorothioate diester linkage, —OP(═O)(SH)O—, which as appreciated by those skilled in the art may exist as a salt form). In some embodiments, an internucleotidic linkage is one of, e.g., PNA (peptide nucleic acid) or PMO (phosphorodiamidate Morpholino oligomer) linkage. In some embodiments, a modified internucleotidic linkage is a non-negatively charged internucleotidic linkage. In some embodiments, a modified internucleotidic linkage is a neutral internucleotidic linkage (e.g., n001 in certain provided oligonucleotides). In some embodiments, a modified internucleotidic linkage comprise no linkage phosphorus (e.g., —C(O)—O— or —C(O)—N(R′)— as described herein). It is understood by a person of ordinary skill in the art that internucleotidic linkages may exist as anions or cations at a given pH due to the existence of acid or base moieties in the linkages.

In certain embodiments, a non-negatively charged internucleotidic linkage comprises a cyclic guanidine moiety. In certain embodiments, a modified internucleotidic linkage comprising a cyclic guanidine moiety has the structure of:

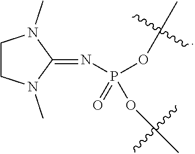 [SMILES: *OP(=O)(N=C1N(C)CCN1C)O*]

In certain embodiments, a neutral internucleotidic linkage comprising a cyclic guanidine moiety is chirally controlled. In certain embodiments, the present disclosure pertains to a composition comprising an oligonucleotide comprising at least one neutral internucleotidic linkage and at least one phosphorothioate internucleotidic linkage.

In certain embodiments, the present disclosure pertains to a composition comprising an oligonucleotide comprising at least one neutral internucleotidic linkage and at least one phosphorothioate internucleotidic linkage, wherein the phosphorothioate internucleotidic linkage is a chirally controlled internucleotidic linkage in the Sp configuration.

In certain embodiments, the present disclosure pertains to a composition comprising an oligonucleotide comprising at least one neutral internucleotidic linkage and at least one phosphorothioate internucleotidic linkage, wherein the phosphorothioate is a chirally controlled internucleotidic linkage in the Rp configuration.

In certain embodiments, the present disclosure pertains to a composition comprising an oligonucleotide comprising at least one neutral internucleotidic linkage of a neutral internucleotidic linkage comprising a Tmg group

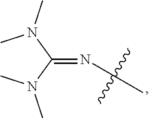 [SMILES: *N=C(N(C)C)N(C)C]

and at least one phosphorothioate.

In certain embodiments, each internucleotidic linkage in an oligonucleotide is independently selected from a natural phosphate linkage, a phosphorothioate linkage, and a non-negatively charged internucleotidic linkage (e.g., n001, n003, n004, n006, n008, n009, n013, n020, n021, n025, n026, n029, n031, n037, n046, n047, n048, n054, or n055). In some embodiments, each internucleotidic linkage in an oligonucleotide is independently selected from a natural phosphate linkage, a phosphorothioate linkage, and a neutral internucleotidic linkage (e.g., n001, n003, n004, n006, n008, n009, n013 n020, n021, n025, n026, n029, n031, n037, n046, n047, n048, n054, or n055). In some embodiments, an oligonucleotide comprises an internucleotidic linkage selected from n001, n002, n003, n004, n006, n008, n009, n012, n013 n020, n021, n024, n025, n026, n029, n030, n031, n033, n034, n035, n036, n037, n041, n043, n044, n046, n047, n048, n051, n052, n054, n055, and n057.

As used herein, the phrase “linkage phosphorus” is used to indicate that the particular phosphorus atom being referred to is the phosphorus atom present in an internucleotidic linkage, which phosphorus atom corresponds to the phosphorus atom of a phosphodiester internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is the P of Formula I as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858). In some embodiments, a linkage phosphorus atom is chiral. In some embodiments, a linkage phosphorus atom is achiral (e.g., as in natural phosphate linkages). In some embodiments, a linkage phosphorus is bonded to a sugar through an oxygen or a nitrogen atom.

Linker: The terms “linker”, “linking moiety” and the like refer to any chemical moiety which connects one chemical moiety to another. As appreciated by those skilled in the art, a linker can be bivalent or trivalent or more, depending on the number of chemical moieties the linker connects. In some embodiments, a linker is a moiety which connects one oligonucleotide to another oligonucleotide in a multimer. In some embodiments, a linker is a moiety optionally positioned between the terminal nucleoside and the solid support or between the terminal nucleoside and another nucleoside, nucleotide, or nucleic acid. In some embodiments, in an oligonucleotide a linker connects a chemical moiety (e.g., a targeting moiety, a lipid moiety, a carbohydrate moiety, etc.) with an oligonucleotide chain (e.g., through its 5′-end, 3′-end, nucleobase, sugar, internucleotidic linkage, etc.)

Modified nucleobase: The terms “modified nucleobase”, “modified base” and the like refer to a chemical moiety which differs structurally from a natural nucleobase but is capable of performing at least one function of a natural nucleobase. In some embodiments, a modified nucleobase is capable of, e.g., forming a moiety in a polymer capable of base-pairing to a nucleic acid comprising a complementary sequence of bases. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. In some embodiments, a modified nucleoside comprises a modified nucleobase. In some embodiments, a modified nucleoside comprises a modified sugar. In some embodiments, a modified nucleoside comprises a modified nucleobase and a modified sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising a complementary sequence of bases.

Modified nucleotide: The term “modified nucleotide” refers to a chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, a modified nucleobase and/or a modified internucleotidic linkage. In some embodiments, a modified nucleotide is capable of, e.g., forming a subunit in a polymer capable of base-pairing to a nucleic acid comprising an at least complementary sequence of bases.

Modified sugar: The term “modified sugar” refers to a moiety which differs structurally from the natural ribose and deoxyribose sugars typically found in natural DNA and RNA and can replace a sugar in an oligonucleotide or a nucleic acid. A modified sugar mimics the spatial arrangement, electronic properties, or some other physicochemical property of a sugar. In some embodiments, as described in the present disclosure, a modified sugar is substituted ribose or deoxyribose. In some embodiments, a modified sugar comprises a 2′-modification. Examples of useful 2′- modification are widely utilized in the art and described herein. In some embodiments, a 2′-modification is 2′-OR, wherein R is optionally substituted C₁₋₁₀ aliphatic. In some embodiments, a 2′-modification is 2′-OMe. In some embodiments, a 2′-modification is 2′-MOE. In some embodiments, a modified sugar is a bicyclic sugar (e.g., a sugar used in LNA, BNA, etc.). In some embodiments, a modified sugar comprises a ring that is not a 5-membered ring. In some embodiments, a modified sugar is acyclic sugar. In some embodiments, a modified sugar comprises a nitrogen atom. In some embodiments, a modified sugar comprises a nitrogen atom, and is bond to an internucleotidic linkage through the nitrogen atom.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleobase: The term “nucleobase” refers to moieties that forms parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a naturally-occurring nucleobases are modified adenine, guanine, uracil, cytosine, or thymine. In some embodiments, a naturally-occurring nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” and is a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides. In some embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, the oligonucleotide is from about 9 to about 39 nucleosides in length. In some embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In some embodiments, the oligonucleotide is at least 4 nucleosides in length. In some embodiments, the oligonucleotide is at least 5 nucleosides in length. In some embodiments, the oligonucleotide is at least 6 nucleosides in length. In some embodiments, the oligonucleotide is at least 7 nucleosides in length. In some embodiments, the oligonucleotide is at least 8 nucleosides in length. In some embodiments, the oligonucleotide is at least 9 nucleosides in length. In some embodiments, the oligonucleotide is at least 10 nucleosides in length. In some embodiments, the oligonucleotide is at least 11 nucleosides in length. In some embodiments, the oligonucleotide is at least 12 nucleosides in length. In some embodiments, the oligonucleotide is at least 15 nucleosides in length. In some embodiments, the oligonucleotide is at least 15 nucleosides in length. In some embodiments, the oligonucleotide is at least 16 nucleosides in length. In some embodiments, the oligonucleotide is at least 17 nucleosides in length. In some embodiments, the oligonucleotide is at least 18 nucleosides in length. In some embodiments, the oligonucleotide is at least 19 nucleosides in length. In some embodiments, the oligonucleotide is at least 20 nucleosides in length. In some embodiments, the oligonucleotide is at least 25 nucleosides in length. In some embodiments, the oligonucleotide is at least 30 nucleosides in length. In some embodiments, the oligonucleotide is a duplex of complementary strands of at least 18 nucleosides in length. In some embodiments, the oligonucleotide is a duplex of complementary strands of at least 21 nucleosides in length. In some embodiments, each nucleoside counted in an oligonucleotide length independently comprises A, T, C, G, or U, or optionally substituted A, T, C, G, or U, or an optionally substituted tautomer of A, T, C, G or U.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, phosphorothioate triester, etc.), pattern of backbone chiral centers [i.e., pattern of linkage phosphorus stereochemistry (Rp/Sp)], and pattern of backbone phosphorus modifications (e.g., pattern of “—XLR¹” groups in Formula I as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858). In some embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. In some embodiments, the present disclosure provides compositions comprising or consisting of a plurality of oligonucleotide molecules (e.g., chirally controlled oligonucleotide compositions). In some embodiments, all such molecules are of the same type (i.e., are structurally identical to one another). In some embodiments, however, provided compositions comprise a plurality of oligonucleotides of different types, typically in pre-determined relative amounts.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R°; —(CH₂)₀₋₄OR°; —O(CH₂)₀₋₄R°, —O—(CH₂)₀₋₄C(O)OR°; —(CH₂)₁₋₄CH(OR°)₂; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; —NO₂; —CN; —N₃;-(CH₂)ₒₜN(R°)₂; —(CH₂)₀₋₄N(R°)C(O)R°); —N(R°)C(S)R°); —(CH₂)₀₋₄N(R°)C(O)NR°₂; —N(R°)C(S)NR°₂; —(CH₂)₀₋₄N(R°)C(O)₀R°);)—N(R°)N(R°)C(O)R°; —N(R°)N(R°)C(O)NR°₂); —N(R°)N(R°)C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°)₃; —(CH₂)₀₋₄OC(O)R°; —OC(O)(CH₂)₀₋₄SR°, —SC(S)SR°; —(CH₂)₀₋₄SC(O)R°; —(CH₂)₀₋₄C(O)NR°₂; —C(S)NR°₂; —C(S)SR°; —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)OR°; —(CH₂)₀₋₄OS(O)₂R°; —S(O)₂NR°₂; —(CH₂)₀₋₄S(O)R°);—N(R°)S(O)₂NR°₂; —N(R°)S(O)₂R°);—N(OR°)R°; —C(NH)NR°₂; —Si(R°)₃; —OSi(R°)₃; —B(R°)₂; —OB(R°)₂; —OB(OR°)₂; —P(R°)₂; —P(OR°)₂; —P(R°)(OR°); —OP(R°)₂; —OP(OR°)₂; —OP(R°)(OR°); —P(O)(R°)₂; —P(O)(OR°)₂; —OP(O)(R°)₂; —OP(O)(OR°)₂; —OP(O)(OR°)(SR°);—SP(O)(R°)₂; —SP(O)(OR°)₂; —N(R°)P(O)(R°)_{2;))}; —N(R°)P(O)(OR°)₂; —P(R°)₂[B(R°)₃]; —P(OR°)₂[B(R°)₃]; —OP(R°)₂[B(R°)₃]; —OP(OR°)₂[B(R°)₃]; —(C₁₋₄ straight or branched) alkylene)O—N(R°)₂; or —(C₁₋₄ straight or branched)alkylene)C(O)O—N(R°)₂, wherein each R° may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, ; —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R°, taken together with their intervening atom(s), form a 5-20 membered, monocyclic, bicyclic, or polycyclic, saturated, partially unsaturated or aryl ring having 05 heteroatoms independently selected from nitrogen, oxygen, sulfur, silicon and phosphorus, which may be substituted as defined below.

Suitable monovalent substituents on R° (or the ring formed by taking two independent occurrences of R° together with their intervening atoms), are independently halogen, —(CH₂)₀₋₂R·,—(halonR·), —(CH₂)₀₋₂OH, —(CH₂)₀₋₂OR°, —(CH₂)₀₋₂CH(OR·)_{2; —0}(haloR*), —CN, —N₃, —(CH₂)₀₋₂C(O)R·,—(CH₂)₀₋₂C(O)OH, —(CH₂)₀₋₂C(O)OR·, —(CH₂)₀₋₂SR·, —(CH₂)₀₋₂SH, —(CH₂)₀₋₂NH₂, —(CH₂)₀₋₂NHR·,—(CH₂)₀₋₂NR·₂, —NO₂, —SiR·₃, —C(O)SR·, —(C₁₋₄ straight or branched alkylene)C(O)OR·, or—SSR· wherein each R· is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R°include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R^{*} are independently halogen, —R·, -(halonR·), —OH, —OR·, —O(halonR·), —CN, —C(O)OH, —C(O)OR·, —NH₂, —NHR·, —NR·₂, or —NO₂, wherein each R· is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R·, (halon·), —OH, —OR·, —O(halonR·), —CN, —C(O)OH, —C(O)OR·, —NH₂, —NHR·, —NR·₂, or —NO₂, wherein each R· is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

P-modification: as used herein, the term “P-modification” refers to any modification at the linkage phosphorus other than a stereochemical modification. In some embodiments, a P-modification comprises addition, substitution, or removal of a pendant moiety covalently attached to a linkage phosphorus.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O— and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Predetermined: By predetermined (or pre-determined) is meant deliberately selected or non-random or controlled, for example as opposed to randomly occurring, random, or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that are not controlled to intentionally generate the particular chemistry and/or stereochemistry features are not “predetermined” compositions. In some embodiments, a predetermined composition is one that can be intentionally reproduced (e.g., through repetition of a controlled process). In some embodiments, a predetermined level of a plurality of oligonucleotides in a composition means that the absolute amount, and/or the relative amount (ratio, percentage, etc.) of the plurality of oligonucleotides in the composition is controlled. In some embodiments, a predetermined level of a plurality of oligonucleotides in a composition is achieved through chirally controlled oligonucleotide preparation.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylypethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridypethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilypethoxy]methylamine (SEM), N-3-acetoxypropylamine, N-(1-isopropyl 4 nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-R4-methoxyphenyl)diphenylmethyllamine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethylene amine, N-benzylideneamine, N-p-methoxybenzylideneamine, N-diphenylmethylene amine, N-[(2-pyridyl)me sityl]methylene amine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzene sulfenamide, o-nitrobenzenesulfenamide (Nps), 2,4-dinitrobenzenesulfenamide, pentachlorobenzene sulfenamide, 2-nitro-4-methoxybenzene sulfenamide, triphenylmethylsulfenamide, 3-nitropyridine sulfenamide (Npys), p-toluene sulfonamide (Ts), benzene sulfonamide, 2,3 ,6,-trimethyl-4-methoxybenzene sulfonamide (Mtr), 2,4,6-trimethoxybenzenesulfonamide (Mtb), 2,6-dimethyl-4-methoxybenzene sulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methane sulfonamide (Ms), β-trimethylsilylethane sulfonamide (SES), 9-anthracene sulfonamide, 4-(4′, 8′-dimethoxynaphthylmethyl)benzene sulfonamide (DNMB S), benzyl sulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3 ,3a,4,5,6,7,7a-octahydro-7, 8 , 8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis (4′,4′-dimethoxyphenyl)methyl, 1, 1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2, 6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2- trimethylsilylethyl, p-chlorophenyl, 2,4-dinitrophenyl, benzyl, benzoyl, p-phenylbenzoyl, 2,6- dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9- fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1, 1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetypamino]butyl.

Subject: As used herein, the term “subject” or “test subject” refers to any organism to which a provided compound (e.g., a provided oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially identical to a second sequence is not identical to the second sequence, but is mostly or nearly identical to the second sequence. In addition, one of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars. In some embodiments, a sugar is a RNA or DNA sugar (ribose or deoxyribose). In some embodiments, a sugar is a modified ribose or deoxyribose sugar, e.g., 2′-modified, 5′-modified, etc. As described herein, in some embodiments, when used in oligonucleotides and/or nucleic acids, modified sugars may provide one or more desired properties, activities, etc. In some embodiments, a sugar is optionally substituted ribose or deoxyribose. In some embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The term “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., oligonucleotides) generally also apply to pharmaceutically acceptable salts of such compounds.

### Description of Certain Embodiments

Oligonucleotides are useful tools for a wide variety of applications. For example, oligonucleotides are useful in various therapeutic, diagnostic, and research applications. Use of naturally occurring nucleic acids (e.g., unmodified DNA or RNA) is limited, for example, by their susceptibility to endo- and exo-nucleases. As such, various synthetic counterparts have been developed to circumvent these shortcomings and/or to further improve various properties and activities. These include synthetic oligonucleotides that contain chemical modifications, e.g., base modifications, sugar modifications, backbone modifications, etc., which, among other things, can render these molecules less susceptible to degradation and improve other properties and/or activities. From a structural point of view, modifications to internucleotidic linkages can introduce chirality, and certain properties may be affected by configurations of linkage phosphorus atoms of oligonucleotides. For example, binding affinity, sequence specific binding to complementary RNA, stability to nucleases, cleavage of target nucleic acids, delivery, pharmacokinetics, etc. can be affected by chirality of backbone linkage phosphorus atoms.

Among other things, the present disclosure provides technologies (e.g., oligonucleotides, compositions, methods, etc.) that comprise various structural elements and/or patterns thereof (e.g., modified sugars, modified internucleotidic linkages, patterns of sugars, patterns of internucleotidic linkages, patters of backbone linkage phosphorus, additional chemical moieties, etc.). With its incorporation and control of various structural elements in oligonucleotides, the present disclosure provides oligonucleotides with improved and/or new properties and/or activities for various applications, e.g., as therapeutic agents, probes, etc. In some embodiments, oligonucleotides of the present disclosure comprise one or more modified sugars and/or modified internucleotidic linkages as described herein. In some embodiments, various internucleotidic linkages oligonucleotides are independently chirally controlled. In some embodiments, the present disclosure provides chirally controlled oligonucleotide compositions in which oligonucleotides comprises various modified sugars (e.g., sugars contain nitrogen atoms and/or acyclic sugars) and/or modified internucleotidic linkages (e.g., those with linkage phosphorus atoms bonded to nitrogen atoms, those of or comprising —C(O)—O— or —C(O)—N(R′)— in which —C(O)— is bonded to a nitrogen atom).

### Sugars

Various sugars, including modified sugars, can be utilized in accordance with the present disclosure. In some embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar is a natural DNA sugar (in DNA nucleic acids or oligonucleotides, having the structure of

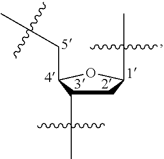

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., typically —OH unless indicated otherwise), and if at the 3′-end of an oligonucleotide, the 3′ position may be connected to a 3′-end group (e.g., typically —OH unless indicated otherwise)). In some embodiments, a sugar is a natural RNA sugar (in RNA nucleic acids or oligonucleotides, having the structure of

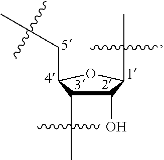

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., typically —OH unless indicated otherwise), and if at the 3′-end of an oligonucleotide, the 3′ position may be connected to a 3′-end group (e.g., typically —OH unless indicated otherwise). In some embodiments, a sugar is a modified sugar in that it is not a natural DNA sugar or a natural RNA sugar. Among other things, modified sugars may provide improved stability. In some embodiments, modified sugars can be utilized to alter and/or optimize one or more hybridization characteristics. In some embodiments, modified sugars can be utilized to alter and/or optimize target recognition. In some embodiments, modified sugars can be utilized to optimize Tm. In some embodiments, modified sugars can be utilized to improve oligonucleotide activities. Sugars can be bonded to internucleotidic linkages at various positions. As non-limiting examples, internucleotidic linkages can be bonded to the 2′, 3′, 4′ or 5′ positions of ribose sugars. In some embodiments, as most commonly in natural nucleic acids, an internucleotidic linkage connects with one sugar at the 5′ position and another sugar at the 3′ position unless otherwise indicated.

In some embodiments, the present disclosure provides oligonucleotides comprising modified sugars comprising nitrogen. In some embodiments, oligonucleotides of the present disclosure comprise combinations of sugars comprising nitrogen, and deoxyribose sugars which are independently and optionally modified as described herein (e.g., 2′-modifications such as R^{2s}, bicyclic sugars comprising bridges between 2′-carbons and carbons at other positions (e.g., 4′-carbons)). In some embodiments, a sugar comprising nitrogen is bonded to an internucleotidic linkage via the nitrogen atom. In some embodiments, an internucleotidic linkage bonded to nitrogen has the structure of —P^{L}(—X—R^{L})—Z—. In some embodiments, an internucleotidic linkage bonded to nitrogen has the structure of —C(O)—O—. In some embodiments, an internucleotidic linkage bonded to nitrogen has the structure of —C(O)—N(R′)—.

In some embodiments, a modified sugar has the structure of

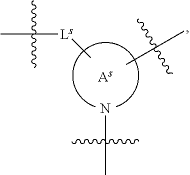

wherein each variable is as described herein. In some embodiments, a sugar is bonded to an internucleotidic linkage, e.g., an internucleotidic linkage having the structure of —P^{L}(—X—R^{L})—Z—, —C(O)—O—, or —C(O)—N(R′)—, at the nitrogen. In some embodiments, Ring As is an optionally substituted 3-30 membered, monocyclic, bicyclic or polycyclic ring having, in addition to the nitrogen, 0-10 heteroatoms. In some embodiments, Ring As is an optionally substituted 3-10 membered, monocyclic, bicyclic or polycyclic ring having, in addition to the nitrogen, 0-10 heteroatoms. In some embodiments, Ring As is an optionally substituted 3-30 membered monocyclic ring having, in addition to the nitrogen, 0-5 heteroatoms. In some embodiments, Ring As is an optionally substituted 3-30 membered monocyclic ring having, in addition to the nitrogen, one heteroatom. In some embodiments, the one heteroatom is oxygen. In some embodiments, Ring As is saturated. In some embodiments, Ring As is optionally substituted

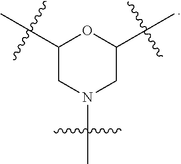 [SMILES: *C1CN(*)CC(*)O1]

In some embodiments, L^{s} is optionally substituted —CH₂—. In some embodiments, L^{s} is —CH₂—. In some embodiments, a modified sugar is optionally substituted

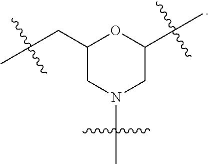 [SMILES: *CC1CN(*)CC(*)O1]

In some embodiments, a modified sugar is

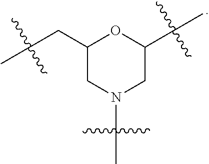 [SMILES: *CC1CN(*)CC(*)O1]

In some embodiments, a modified sugar is optionally substituted

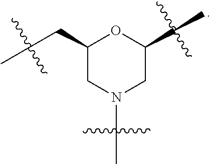 [SMILES: *C[C@@H]1CN(*)C[C@H](*)O1]

In some embodiments, a modified sugar is

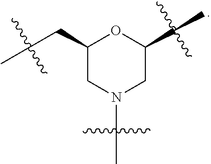 [SMILES: *C[C@@H]1CN(*)C[C@H](*)O1]

In some embodiments, a modified sugar has the structure of

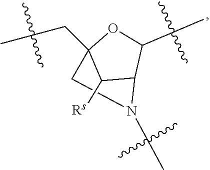

wherein R^{s} is as described herein. In some embodiments, a modified sugar is

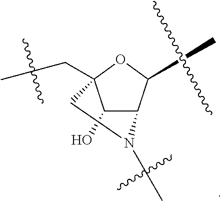

In some embodiments, a modified sugar is

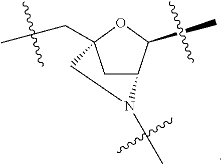

In some embodiments, a nucleoside is Asm01, Tsm01, Csm01, Gsm01, in which the sugar is

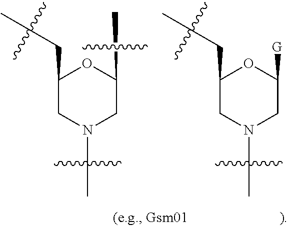

In some embodiments, an oligonucleotide comprises one or more (e.g., 1-20, 1-15, 1-10, 1-8, 1-5, 1-4, 1-3, 1, 2, 3, 4, 5, 6, 7, 8, 9, or 10) sm01. In some embodiments, an oligonucleotide contains no more than 10 sm01. In some embodiments, an oligonucleotide contains no more than 9 sm01. In some embodiments, an oligonucleotide contains no more than 8 sm01. In some embodiments, an oligonucleotide contains no more than 7 sm01. In some embodiments, an oligonucleotide contains no more than 6 sm01. In some embodiments, an oligonucleotide contains no more than 5 sm01. In some embodiments, an oligonucleotide contains no more than 4 sm01. In some embodiments, an oligonucleotide contains no more than 3 sm01. In some embodiments, an oligonucleotide contains no more than 10 consecutive sm01. In some embodiments, an oligonucleotide contains no more than 9 consecutive sm01. In some embodiments, an oligonucleotide contains no more than 8 consecutive sm01. In some embodiments, an oligonucleotide contains no more than 7 consecutive sm01. In some embodiments, an oligonucleotide contains no more than 6 consecutive sm01. In some embodiments, an oligonucleotide contains no more than 5 consecutive sm01. In some embodiments, an oligonucleotide contains no more than 4 consecutive sm01. In some embodiments, an oligonucleotide contains no more than 3 consecutive sm01. In some embodiments, one or more sm01 are each independently bonded at its nitrogen atom to a linkage whose linkage phosphorus is bonded to another nitrogen (e.g., as in sm01n001). In some embodiments, each sm01 is independently bonded at its nitrogen atom to a linkage whose linkage phosphorus is bonded to another nitrogen (e.g., as in sm01n001).

In some embodiments, a modified sugar is an acyclic sugar. In some embodiments, an acyclic sugar has the structure of a′-L^{SA1}-L^{SA2}(-L^{SA3}-)-L^{SA4}-b′, wherein each of L^{SA1}, L^{SA3}, and L^{SA4} is independently optionally substituted bivalent C₁₋₄ aliphatic or C₁₋₄ aliphatic having 1-3 heteroatoms, and L^{SA2} is optionally substituted CH or N. In some embodiments, wherein each of L^{SA1}, L^{SA3}, and L^{SA4} is independently optionally substituted bivalent C₁₋₂ aliphatic or C₁₋₂ aliphatic having 1-2 heteroatoms. In some embodiments, L^{SA3} is bonded to a nucleobase. In some embodiments, L^{SA1} is optionally substituted —CH₂—. In some embodiments, L^{SA1} ^{is ; —CH}₂—. In some embodiments, L^{SA1} is —CH(CH₃)—. In some embodiments, L^{SA1} is optionally substituted —CH₂CH₂—. In some embodiments, L^{SA1} is —CH₂CH₂—. In some embodiments, L^{SA1} is optionally substituted —CH₂NH₂—. In some embodiments, L^{SA1} is —CH₂NH₂—. In some embodiments, L^{SA2} is optionally substituted CH. In some embodiments, L^{SA2} is optionally substituted N. In some embodiments, L^{SA3} is optionally substituted —O—CH₂—. In some embodiments, L^{SA3} is —O—CH(CH₃)—. In some embodiments, L^{SA3} is —O—CH(CH₂OH)—. In some embodiments, L^{SA3} is optionally substituted —C(O)—CH₂—. In some embodiments, L^{SA3} is —C(O)—CH₂—. In some embodiments, LSA4 is optionally substituted —CH₂—. In some embodiments, L^{SA4} is n some embodiments, L^{SA4} is —CH(CH₃)—. In some embodiments, L^{SA4}is optionally substituted —CH₂CH₂—. In some embodiments, L^{SA4} is —CH₂CH₂—. In some embodiments, L^{SA4}is optionally substituted —CH₂NH₂—. In some embodiments, L′ is —CH₂NH₂—. In some embodiments, an acyclic sugar has the structure of a′-CH₂; —CH(-L^{SA3}-)—CH₂-b′, wherein each of the CH₂ and CH is independently optionally substituted. In some embodiments, L^{SA3} is —O—CH₂—, wherein the CH₂ is optionally substituted. In some embodiments, an acyclic sugar has the structure of a′-CH₂; —CH(—O—CH₂)—CH₂-b′, wherein each of the CH₂ and CH is independently optionally substituted. In some embodiments, an acyclic sugar has the structure of a′-CH₂; —CH(—O—CH₂)—CH₂-b′. In some embodiments, an acyclic sugar has the structure of a′-CH₂; —CH(—O—CH₂)—CH(CH₃)-b′, wherein each of the CH₂ and CH is independently optionally substituted. In some embodiments, an acyclic sugar has the structure of a′-CH₂; —CH(—O—CH₂)—CH(CH₃)-b′. In some embodiments, an acyclic sugar has the structure of a′-CH₂; —CH(—O—CH(CH₃)—)—CH₂-b′, wherein each of the CH₂ and CH is independently optionally substituted. In some embodiments, an acyclic sugar has the structure of a′; —CH₂CH(—O—CH(CH₃)—)—CH₂-b′. In some embodiments, an acyclic sugar has the structure of a′-CH₂; —CH(—O—CH(CH₂OH)—)—CH₂-b′, wherein each of the CH₂ and CH is independently optionally substituted. In some embodiments, an acyclic sugar has the structure of a′-CH₂; —CH(—O—CH(CH₂OH)—)—CH₂-b′. In some embodiments, an acyclic sugar has the structure of a′-CH₂; —CH(O—CH₂)—CH₂; —NHR′-b′, wherein each of the CH₂ and CH is independently optionally substituted. In some embodiments, an acyclic sugar has the structure of a′-CH₂; —CH(O—CH₂)—CH₂; —NHR′-b′. In some embodiments, an acyclic sugar has the structure of a′-CH₂; —CH(O—CH₂)—CH₂; —NH₂-b′, wherein each of the CH₂, NH₂ and CH is independently optionally substituted. In some embodiments, an acyclic sugar has the structure of a′-CH₂; —CH(O—CH₂)—CH₂; —NH₂-b′. In some embodiments, an acyclic sugar has the structure of a′-CH₂; —N[—C(O)—CH₂-]—CH₂CH₂-b′, wherein each of the CH₂ and CH is independently optionally substituted. In some embodiments, an acyclic sugar has the structure of a′-CH₂; —N[—C(O)—CH₂; —]—CH₂CH₂-b′. In some embodiments, a′ is the 5′-end. In some embodiments, b′ is the 5′-end.

In some embodiments, an acyclic sugar is

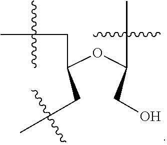

In some embodiments, In some embodiments, a nucleoside is Asm04, Tsm04, Csm04, Gsm04, in which the sugar is

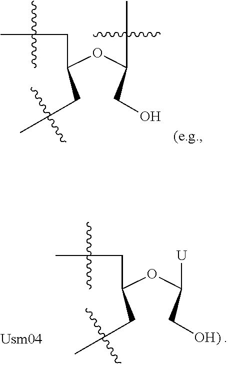

In some embodiments, an acyclic sugar is

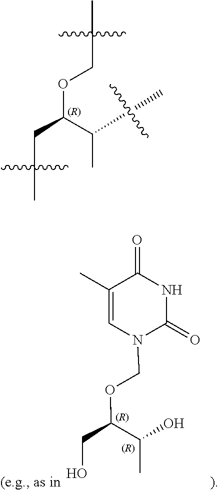

In some embodiments, an acyclic sugar is

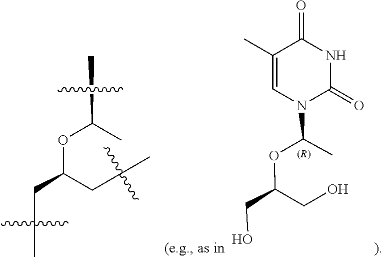

In some embodiments, a modified sugar has the structure of

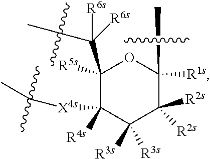

wherein X^{4s} is —O— or —N(R^{4s})—, and each of R^{1s}, R^{2s}, R^{3s}, R^{4s}, R^{5s} and R^{6s} is independently R^{s} as described herein. In some embodiments, X^{4s} is —N(R^{4s})—. In some embodiments, X^{4s} is —NH—. In some embodiments, a modified sugar has the structure of

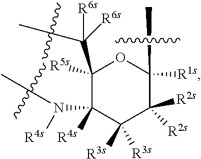

wherein each variable is independently as described herein. In some embodiments, a modified sugar has the structure of

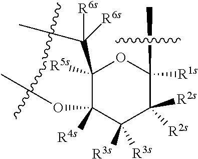

wherein each variable is independently as described herein. In some embodiments, a modified sugar has the structure of

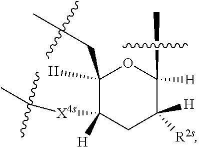

wherein each variable is independently as described herein. In some embodiments, a modified sugar has the structure of

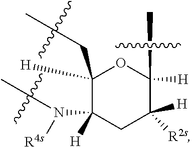

wherein each variable is independently as described herein. In some embodiments, a modified sugar has the structure of

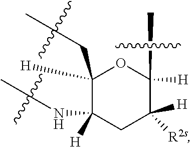

wherein R^{2s} is as described herein. In some embodiments, a modified sugar has the structure of

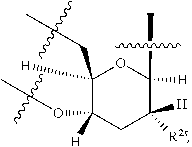

wherein R^{2s} is as described herein.

Various types of sugars may be utilized in accordance with the present disclosure. Sugars comprising nitrogen and/or acyclic sugars are typically utilized together with other types of sugars, e.g., one or more natural sugars (in some embodiments, natural DNA sugars) and one or more other types of modified sugars (e.g., substituted

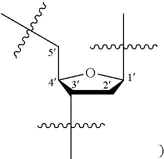

that are not the typical natural DNA or RNA sugars. In some embodiments, oligonucleotides comprise one or more natural DNA sugars. In some embodiments, oligonucleotides comprise one or more natural RNA sugars. In some embodiments, oligonucleotides comprise one or more modified sugars. In some embodiments, a sugar is an optionally substituted natural DNA or RNA sugar. In some embodiments, a sugar is optionally substituted

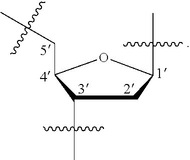 [SMILES: *C[C@H]1O[C@@H](*)C[C@H]1*]

In some embodiments, the 2′ position is optionally substituted. In some embodiments, a sugar is

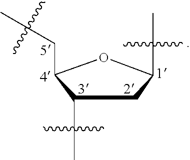 [SMILES: *C[C@H]1O[C@@H](*)C[C@H]1*]

In some embodiments, a sugar has the structure of

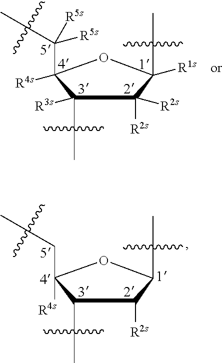

wherein each of R^{1s}, R^{2s}, R^{3s}, R^{4s}, and R^{5s} is independently as described herein. In some embodiments, each of R^{1s}, R^{2s}, R^{3s}, R^{4s}, and R^{5s} is independently -, a suitable substituent or suitable sugar modification (e.g., those described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the substituents, sugar modifications, descriptions of R^{1s}, R^{2s}, R^{3s}, R^{4s}, and R^{5s}, and modified sugars of each of which are independently incorporated herein by reference). In some embodiments, each of R^{1s}, R^{2s}, R^{3s}, R^{4s}, and R^{5s} is independently R^{s}, wherein each R^{s} is independently —H, —F, —Cl, —Br, —I, —CN, —N₃, —NO, —NO₂, -L-R′, -L-OR′, -L-SR′, -L-N(R′)₂, —O-L-OR′, —O-L-SR′, or —O-L-N(R′)₂, wherein each R′ is independently as described herein, and each L is independently a covalent bond or optionally substituted bivalent C₁₋₆ aliphatic or heteroaliphatic having 1-4 heteroatoms; or two R^{s} are taken together to form a bridge -L-. In some embodiments, R′ is optionally substituted C₁₋₁₀ aliphatic. In some embodiments, a sugar has the structure of

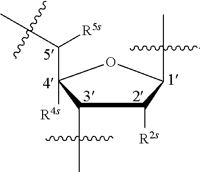

In some embodiments, a sugar has the structure of

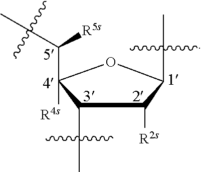

In some embodiments, a sugar has the structure of

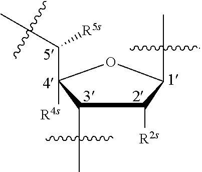

In some embodiments, a sugar has the structure of

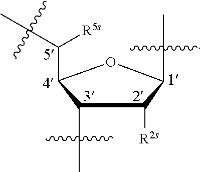

In some embodiments, a sugar has the structure of

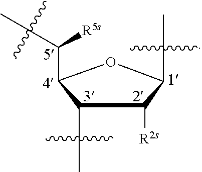

In some embodiments, a sugar has the structure of

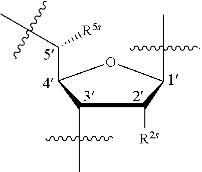

In some embodiments, a sugar has the structure of

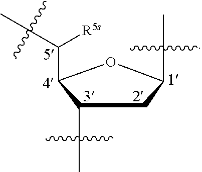

In some embodiments, a sugar has the structure of

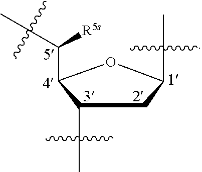

In some embodiments, a sugar has the structure of

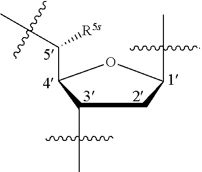

In some embodiments, R^{5s} is optionally substituted C₁₋₆ aliphatic. In some embodiments, R^{5s} is optionally substituted C₁₋₆ alkyl. In some embodiments, R^{5s} is optionally substituted methyl. In some embodiments, R^{5s} is methyl. In some embodiments, a sugar has the structure of

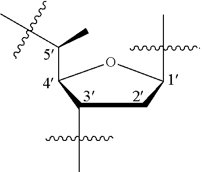 [SMILES: *[C@@H]1C[C@H](*)O[C@@H]1[C@@H](*)C]

In some embodiments, a sugar has the structure of

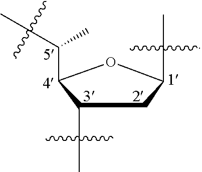 [SMILES: *[C@H](C)[C@H]1O[C@@H](*)C[C@H]1*]

In some embodiments, a sugar has the structure of

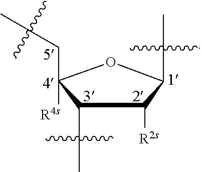

In some embodiments, R^{4s} is —H. In some embodiments, a sugar has the structure of

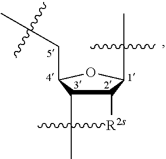

wherein R^{2s} is —H, halogen, or —OR, wherein R is optionally substituted C₁₋₆ aliphatic. In some embodiments, R^{2s} is —H. In some embodiments, R^{2s} is —F. In some embodiments, a modified nucleoside is fA, fr, fC, fG, fU, etc., in which R^{2s} is —F. In some embodiments, R^{2s} is —OMe. In some embodiments, a modified nucleoside is mA, mT, mC, m5 mC, mG, mU, etc., in which R^{2s} is —OMe. In some embodiments, R^{2s} is —OCH₂CH₂OMe. In some embodiments, a modified nucleoside is Aeo, Teo, Ceo, m5Ceo, Geo, Ueo, etc., in which R^{2s} is —OCH₂CH₂OMe. In some embodiments, R^{2s} is —OCH₂CH₂OH. In some embodiments, an oligonucleotide comprises a 2′-F modified sugar having the structure of

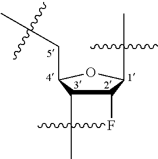

(e.g., as in fA, fr, fC, f5 mC, fG, fU, etc.). In some embodiments, an oligonucleotide comprises a 2′-OMe modified sugar having the structure of

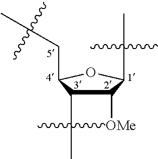

(e.g., as in mA, mT, mC, m5 mC, mG, mU, etc.). In some embodiments, an oligonucleotide comprises a 2′-MOE modified sugar having the structure of

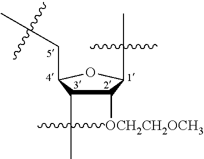

(e.g., as in Aeo, Teo, Ceo, m5Ceo, Geo, Ueo, etc.).

In some embodiments, a sugar has the structure of

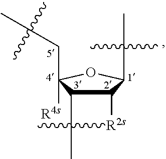

wherein R^{2s} and R^{4s} are taken together to form -L-, wherein L is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L is optionally substituted C2; —O—CH₂; —C4. In some embodiments, L is C2; —O—CH₂; —C4. In some embodiments, L is C2; —O—(R)—CH(CH₂CH₃)—C4. In some embodiments, L is C2; —O—(S)—CH(CH₂CH₃)—C4.

In some embodiments, a sugar comprises a 5′-modification. In some embodiments, one R^{5s} is R, wherein R is optionally substituted C₁₋₆ aliphatic. In some embodiments, R is methyl. In some embodiments, it is 5′-(R)-methyl. In some embodiments, it is 5′-(S)-methyl.

In some embodiments, a sugar is a bicyclic sugar, e.g., sugars wherein R^{2s} and R^{4s} are taken together to form a link as described in the present disclosure. In some embodiments, a sugar is selected from LNA sugars, BNA sugars, cEt sugars, etc. In some embodiments, a bridge is between the 2′ and 4′-carbon atoms (corresponding to R^{2s} and R^{4s} taken together with their intervening atoms to form an optionally substituted ring as described herein). In some embodiments, examples of bicyclic sugars include alpha-L-methyleneoxy (4′-CH₂-O-2′) LNA, beta-D-methyleneoxy (4′-CH₂-O-2′) LNA, ethyleneoxy (4′-(CH₂)₂-O-2′) LNA, aminooxy (4′-CH₂-O—N(R)-2′) LNA, and oxyamino (4′-CH₂; —N(R)-O-2′) LNA. In some embodiments, a bicyclic sugar, e.g., a LNA or BNA sugar, is sugar having at least one bridge between two sugar carbons. In some embodiments, a bicyclic sugar in a nucleoside may have the stereochemical configurations of alpha-L-ribofuranose or beta-D-ribofuranose. In some embodiments, a sugar is a sugar described in WO 1999014226. In some embodiments, a 4′-2′ bicyclic sugar or 4′ to 2′ bicyclic sugar is a bicyclic sugar comprising a furanose ring which comprises a bridge connecting the 2′ carbon atom and the 4′ carbon atom of the sugar ring. In some embodiments, a bicyclic sugar, e.g., a LNA or BNA sugar, comprises at least one bridge between two pentofuranosyl sugar carbons. In some embodiments, a LNA or BNA sugar, comprises at least one bridge between the 4′ and the 2′ pentofuranosyl sugar carbons.

In some embodiments, a bicyclic sugar is a sugar of alpha-L-methyleneoxy (4′-CH₂; —O-2′) BNA, beta-D-methyleneoxy (4′-CH₂-O-2′) BNA, ethyleneoxy (4′—(CH₂)₂; —O-2′) BNA, aminooxy (4′-CH₂; —O—N(R)-2′) BNA, oxyamino (4′-CH₂; —N(R)—O-2′) BNA, methyl(methyleneoxy) (4′-CH(CH₃)—O-2′) BNA (also referred to as constrained ethyl or cEt), methylene-thio (4′-CH₂; —S-2′) BNA, methylene-amino (4′-CH₂; —N(R)-2′) BNA, methyl carbocyclic (4′—CH₂; —CH(CH₃)-2′) BNA, propylene carbocyclic (4′—(CH₂)₃-2′) BNA, or vinyl BNA.

In some embodiments, a sugar modification is 2′-OMe, 2′-MOE, 2′-LNA, 2′-F, 5′-vinyl, or S-cEt. In some embodiments, a modified sugar is a sugar of FRNA, FANA, or morpholino. In some embodiments, an oligonucleotide comprises a nucleic acid analog, e.g., GNA, LNA, PNA, TNA, F-HNA (F-THP or 3′-fluoro tetrahydropyran), MNA (mannitol nucleic acid, e.g., Leumann 2002 Bioorg. Med. Chem. 10: 841-854), ANA (anitol nucleic acid), or morpholino, or a portion thereof. In some embodiments, a sugar modification replaces a natural sugar with another cyclic or acyclic moiety. Examples of such moieties are widely known in the art, e.g., those used in morpholino, glycol nucleic acids, etc. and may be utilized in accordance with the present disclosure. As appreciated by those skilled in the art, when utilized with modified sugars, in some embodiments internucleotidic linkages may be modified, e.g., as in morpholino, PNA, etc.

In some embodiments, a sugar is a 6′-modified bicyclic sugar that have either (R) or (S)-chirality at the 6-position, e.g., those described in U.S. Pat. No. 7,399,845. In some embodiments, a sugar is a 5′-modified bicyclic sugar that has either (R) or (S)-chirality at the 5-position, e.g., those described in US 20070287831.

In some embodiments, a modified sugar contains one or more substituents at the 2′ position (typically one substituent, and often at the axial position) independently selected from —F; —CF₃, —CN, —N₃, —NO, —NO₂, —OR′, —SR′, or —N(R′)₂, wherein each R′ is independently optionally substituted C₁₋₁₀ aliphatic; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S— (C₂-C₁₀ alkynyl), —NH—(C₂-C₁₀ alkynyl), or —N(C₂-C₁₀ alkynyl)₂; or —O--(C₁-C₁₀ alkylene)-O--(C₁-C₁₀ alkyl), —O—(C₁-C₁₀ alkylene)—NH—(C₁-C₁₀ alkyl) or —O—(C₁-C₁₀ alkylene)—NH(C₁-C₁₀ alkyl)₂, —NH—(C₁-C₁₀ alkylene)—O—(C₁-C₁₀ alkyl), or —N(C₁-C₁₀ alkyl)—(C₁-C₁₀ alkylene)—O—(C₁-C₁₀ alkyl), wherein each of the alkyl, alkylene, alkenyl and alkynyl is independently and optionally substituted. In some embodiments, a substituent is —O(CH₂)₁₁₀CH3, —0(CH₂)₁₁NH₂, MOE, DMAOE, or DMAEOE, wherein wherein n is from 1 to about 10. In some embodiments, a modified sugar is one described in WO 2001/088198; and Martin et al., Hely. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the 2′, 3′, 4′, or 5′ positions, including the 3′ position of the sugar on the 3′-terminal nucleoside or in the 5′ position of the 5′-terminal nucleoside.

In some embodiments, the 2′-OH of a ribose is replaced with a group selected from —H, —F;—CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O— (C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)— O—(C₁-C₁₀ alkyl), —O—(C₁-C₁₀ alkylene)—NH—(C₁-C₁₀ alkyl) or —O—(C₁-C₁₀ alkylene)—NH(C₁-C₁₀ alkyl)₂, —NH—(C₁-C₁₀ alkylene)—O—(C₁-C₁₀ alkyl), or —N(C₁-C₁₀ alkyl)—(C₁-C₁₀ alkylene)—O—(C₁-C₁₀ alkyl), wherein each of the alkyl, alkylene, alkenyl and alkynyl is independently and optionally substituted. In some embodiments, the 2′-OH is replaced with —H (deoxyribose). In some embodiments, the 2′-OH is replaced with —F. In some embodiments, the 2′-OH is replaced with —OR′. In some embodiments, the 2′- OH is replaced with —OMe. In some embodiments, the 2′-OH is replaced with —OCH₂CH₂OMe.

In some embodiments, a sugar modification is a 2′-modification. Commonly used 2′-modifications include but are not limited to 2′-OR, wherein R is optionally substituted C₁₋₆ aliphatic. In some embodiments, a modification is 2′-OR, wherein R is optionally substituted C₁₋₆ alkyl. In some embodiments, a modification is 2′-OMe. In some embodiments, a modification is 2′-MOE. In some embodiments, a 2′-modification is S-cEt. In some embodiments, a modified sugar is an LNA sugar. In some embodiments, a 2′-modification is —F. In some embodiments, a 2′-modification is FANA. In some embodiments, a 2′-modification is FRNA. In some embodiments, a sugar modification is a 5′-modification, e.g., 5′-Me. In some embodiments, a sugar modification changes the size of the sugar ring. In some embodiments, a sugar modification is the sugar moiety in FHNA. In some embodiments, a 2′-modification is 2′-F.

In some embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In some embodiments, 5% or more of the sugars of an oligonucleotide are modified. In some embodiments, 10% or more of the sugars of an oligonucleotide are modified. In some embodiments, 15% or more of the sugars of an oligonucleotide are modified. In some embodiments, 20% or more of the sugars of an oligonucleotide are modified. In some embodiments, 25% or more of the sugars of an oligonucleotide are modified. In some embodiments, 30% or more of the sugars of an oligonucleotide are modified. In some embodiments, 35% or more of the sugars of an oligonucleotide are modified. In some embodiments, 40% or more of the sugars of an oligonucleotide are modified. In some embodiments, 45% or more of the sugars of an oligonucleotide are modified. In some embodiments, 50% or more of the sugars of an oligonucleotide are modified. In some embodiments, 55% or more of the sugars of an oligonucleotide are modified. In some embodiments, 60% or more of the sugars of an oligonucleotide are modified. In some embodiments, 65% or more of the sugars of an oligonucleotide are modified. In some embodiments, 70% or more of the sugars of an oligonucleotide are modified. In some embodiments, 75% or more of the sugars of an oligonucleotide are modified. In some embodiments, 80% or more of the sugars of an oligonucleotide are modified. In some embodiments, 85% or more of the sugars of an oligonucleotide are modified. In some embodiments, 90% or more of the sugars of an oligonucleotide are modified. In some embodiments, 95% or more of the sugars of an oligonucleotide are modified. In some embodiments, each sugar of an oligonucleotide is independently modified. In some embodiments, a modified sugar comprises a 2′-modification. In some embodiments, each modified sugar independently comprises a 2′-modification. In some embodiments, a 2′-modification is 2′-OR¹. In some embodiments, a 2′-modification is a 2′-OMe. In some embodiments, a 2′-modification is a 2′-MOE. In some embodiments, a 2′-modification is an LNA sugar modification. In some embodiments, a 2′-modification is 2′-F. In some embodiments, each sugar modification is independently a 2′-modification. In some embodiments, each sugar modification is independently 2′-OR¹ or 2′-F. In some embodiments, each sugar modification is independently 2′-OR¹ or 2′-F, wherein R¹ is optionally substituted C₁₋₆ alkyl. In some embodiments, each sugar modification is independently 2′-OR¹ or 2′-F, wherein at least one is 2′-F. In some embodiments, each sugar modification is independently 2′-OR¹ or 2′-F, wherein R¹ is optionally substituted C₁₋₆ alkyl, and wherein at least one is 2′-OR¹. In some embodiments, each sugar modification is independently 2′-OR′ or 2′-F, wherein at least one is 2′-F, and at least one is 2′-OR¹. In some embodiments, each sugar modification is independently 2′-OR¹ or 2′-F, wherein R¹ is optionally substituted C₁₋₆ alkyl, and wherein at least one is 2′-F, and at least one is 2′-OR¹. In some embodiments, each sugar modification is independently 2′-OR¹. In some embodiments, each sugar modification is independently 2′-OR¹, wherein R¹ is optionally substituted C₁₋₆ alkyl. In some embodiments, each sugar modification is 2′-OMe. In some embodiments, each sugar modification is 2′-MOE. In some embodiments, each sugar modification is independently 2′-OMe or 2′-MOE. In some embodiments, each sugar modification is independently 2′-OMe, 2′-MOE, or a LNA sugar.

In some embodiments, each sugar independently comprises a 2′-F or 2′-OR modification, wherein R is independently C₁₋₆ aliphatic. In some embodiments, R is —CH₃.

In some embodiments, an oligonucleotide is or comprises a structure of 5′-a first region-a second region-a third region, each of which independently comprises one or more (e.g., 1-30, e.g., about or at least about 1, 2, 3, 4, 5, 6, 7, 8, 9, 10, 11, 12, 13, 14, 15, 16, 17, 18, 19 or 20) nucleobases. In some embodiments, a first region comprises two or more (e.g., 2-10, e.g. about or at least about 2, 3, 4, 5, 6, 7, 8, 9, or 10) nucleobases. In some embodiments, a second region comprises two or more (e.g., 2-20, 5-20, 6-20, 7-20, 8-20, e.g. about or at least about 2, 3, 4, 5, 6, 7, 8, 9, 10, 11, 12, 13, 14, 15, 16, 17, 18, 19, or 20) nucleobases. In some embodiments, a third region comprises two or more (e.g., 2-10, e.g. about or at least about 2, 3, 4, 5, 6, 7, 8, 9, or 10) nucleobases.

In some embodiments, one or more (1-50, 1-40, 1-30, 1-25, 1-20, e.g., 1, 2, 3, 4, 5, 6, 7, 8, 9, 10, 11, 12, 13, 14, 15, 16, 17, 18, 19, 20, 21, 22, 23, 24, or 25, or more) sugars in an oligonucleotide comprise 2′-F modification. In some embodiments, at least 10%, 20%, 30%, 40%, 50%, 60%, 70%, 75%, 80%, 85%, 90%, or 95%, or 100% of all sugars in an oligonucleotide comprise a 2′-F modification. In some embodiments, each of the regions independently comprises one or more (1-50, 1-40, 1-30, 1-25, 1-20, e.g., 1, 2, 3, 4, 5, 6, 7, 8, 9, 10, 11, 12, 13, 14, 15, 16, 17, 18, 19, 20, 21, 22, 23, 24, or 25, or more) sugars comprises 2′-F modification. In some embodiments, at least 10%, 20%, 30%, 40%, 50%, 60%, 70%, 75%, 80%, 85%, 90%, or 95%, or 100% of all sugars in each of the regions independently comprise a 2′-F modification. In some embodiments, the number of 2′-F modified sugars in an oligonucleotide or a region is 2 or more. In some embodiments, it is 3 or more. In some embodiments, it is 4 or more. In some embodiments, it is 5 or more. In some embodiments, it is 6 or more. In some embodiments, it is 7 or more. In some embodiments, it is 8 or more. In some embodiments, it is 9 or more. In some embodiments, it is 10 or more. In some embodiments, the percentage of 2′-F modified sugars in an oligonucleotide or a region is 50% or more. In some embodiments, it is 60% or more. In some embodiments, it is 70% or more. In some embodiments, it is 80% or more. In some embodiments, it is 90% or more. In some embodiments, it is 95% or more. In some embodiments, it is 100%. In some embodiments, two or more or all 2′-F modified sugars are consecutive.

In some embodiments, a first region comprises 1, 2, 3, 4, 5, 6, 7, 8, 9, or more 2′-F modified sugars. In some embodiments, a first region comprises 5, 6, 7, or 8 2′-F modified sugars. In some embodiments, 50%, 60%, 70%, 75%, 80%, 85%, 90%, or 95%, or 100% of all sugars in a first region comprise 2′-F. In some embodiments, each sugar is a first region comprises 2′-F. In some embodiments, a first region comprises one or more (e.g., 1, 2, 3, 4, 5, 6, 7, 8, 9, 10 or more; in some embodiments, 5 or more) phosphorothioate internucleotidic linkages. In some embodiments, each phosphorothioate internucleotidic linkage in a first region is independently chirally controlled and is Sp. In some embodiments, a first region comprises one or more (e.g., 1, 2, 3, 4, 5, 6, 7, 8, 9, 10 or more) non-negatively charged internucleotidic linkages. In some embodiments, each non-negatively charged internucleotidic linkage in a first region is chirally controlled. In some embodiments, one or more non-negatively charged internucleotidic linkage in a first region is not chirally controlled. In some embodiments, each non-negatively charged internucleotidic linkage in a first region is chirally controlled and is Rp. In some embodiments, two or more or all 2′-F modified sugars in a first region are consecutive.

In some embodiments, a second region comprises 1, 2, 3, 4, 5, 6, 7, 8, 9, or more 2′-F modified sugars. In some embodiments, a second region comprises 5, 6, 7, or 8 2′-F modified sugars. In some embodiments, 50%, 60%, 70%, 75%, 80%, 85%, 90%, or 95%, or 100% of all sugars in a second region comprise 2′-F. In some embodiments, each sugar is a second region comprises 2′-F. In some embodiments, a second region comprises one or more (e.g., 1, 2, 3, 4, 5, 6, 7, 8, 9, 10 or more; in some embodiments, 5 or more) phosphorothioate internucleotidic linkages. In some embodiments, each phosphorothioate internucleotidic linkage in a second region is independently chirally controlled and is Sp. In some embodiments, a second region comprises one or more (e.g., 1, 2, 3, 4, 5, 6, 7, 8, 9, 10 or more) non-negatively charged internucleotidic linkages. In some embodiments, each non-negatively charged internucleotidic linkage in a second region is chirally controlled. In some embodiments, one or more non-negatively charged internucleotidic linkage in a second region is not chirally controlled. In some embodiments, each non-negatively charged internucleotidic linkage in a second region is chirally controlled and is Rp. In some embodiments, each internucleotidic linkage in a second region is independently a phosphorothioate internucleotidic linkage. In some embodiments, two or more or all 2′-F modified sugars in a second region are consecutive. In some embodiments, a second region comprises one or more (e.g., 1, 2, 3, 4, 5, 6, 7, 8, 9, 10 or more) sugars that are not 2′-F modified. In some embodiments, one or more (e.g., 1, 2, 3, 4, 5, 6, 7, 8, 9, 10 or more) or all sugars that are not 2′-F modified are 2′-OR modified, wherein R is optionally substituted C₁₋₆ aliphatic. In some embodiments, a second region comprises alternating 2′-F modified sugars and 2′-OR modified sugars, wherein R is optionally substituted C₁₋₆ aliphatic. In some embodiments, the first sugar in a second region (from 5′ to 3′) is a 2′-OR modified sugar, wherein R is optionally substituted C₁₋₆ aliphatic. In some embodiments, the last sugar in a second region (from 5′ to 3′) is a 2′-OR modified sugar, wherein R is optionally substituted C₁₋₆ aliphatic. In some embodiments, both the first and last sugars in a second region are independently a 2′-OR modified sugar, wherein R is optionally substituted C₁₋₆ aliphatic. In some embodiments, R is methyl.

In some embodiments, a third region comprises 1, 2, 3, 4, 5, 6, 7, 8, 9, or more 2′-F modified sugars. In some embodiments, a third region comprises 5, 6, 7, or 8 2′-F modified sugars. In some embodiments, 50%, 60%, 70%, 75%, 80%, 85%, 90%, or 95%, or 100% of all sugars in a third region comprise 2′-F. In some embodiments, each sugar is a third region comprises 2′-F. In some embodiments, a third region comprises one or more (e.g., 1, 2, 3, 4, 5, 6, 7, 8, 9, 10 or more; in some embodiments, 5 or more) phosphorothioate internucleotidic linkages. In some embodiments, each phosphorothioate internucleotidic linkage in a third region is independently chirally controlled and is Sp. In some embodiments, a third region comprises one or more (e.g., 1, 2, 3, 4, 5, 6, 7, 8, 9, 10 or more) non-negatively charged internucleotidic linkages. In some embodiments, each non-negatively charged internucleotidic linkage in a third region is chirally controlled. In some embodiments, one or more non-negatively charged internucleotidic linkage in a third region is not chirally controlled. In some embodiments, each non-negatively charged internucleotidic linkage in a third region is chirally controlled and is Rp. In some embodiments, two or more or all 2′-F modified sugars in a third region are consecutive.

In some embodiments, one or more (1-50, 1-40, 1-30, 1-25, 1-20, e.g., 1, 2, 3, 4, 5, 6, 7, 8, 9, 10, 11, 12, 13, 14, 15, 16, 17, 18, 19, 20, 21, 22, 23, 24, or 25, or more) sugars comprises 2′-F modification.

Among other things, oligonucleotides comprising 2′-F modified sugars are useful for modulating splicing. In some embodiments, the present disclosure provides technologies to incorporating nitrogen-containing sugars, either cyclic or acyclic, into such oligonucleotides, e.g., in first, second and/or third regions. As demonstrated herein, provided oligonucleotides can provide various activities while bearing certain sugars (e.g., sugars comprising nitrogen) and/or internucleotidic linkages (those comprising nitrogen) and/or additional chemical moieties) for modulating and/or optimizing one or more properties (e.g., charges, delivery, distribution, binding strength, etc.).

In some embodiments, provided oligonucleotides comprise portions that can form DNA-RNA duplexes with RNA molecules. Such oligonucleotides may be useful, for example, RNase H-associated activities.

In some embodiments, a first region is referred to as a 5′-wing, a second region is referred to as a core, and a third region is referred to as a 3′-wing. In some embodiments, a wing comprises a sugar modification or a pattern thereof that is absent from a core. In some embodiments, a wing comprises a sugar modification that is absent from a core. In some embodiments, each sugar in a wing is the same. In some embodiments, at least one sugar in a wing is different from another sugar in the wing. In some embodiments, one or more sugar modifications and/or patterns of sugar modifications in a first wing of an oligonucleotide (e.g., a 5′-wing) is/are different from one or more sugar modifications and/or patterns of sugar modifications in a second wing of the oligonucleotide (e.g., a 3′-wing). In some embodiments, a modification is a 2′-OR modification, wherein R is as described herein. In some embodiments, R is optionally substituted C₁₋₄ alkyl. In some embodiments, a modification is 2′-OMe. In some embodiments, a modification is a 2′-MOE. In some embodiments, a modified sugar is a high-affinity sugar, e.g., a bicyclic sugar (e.g., a LNA sugar), 2′-MOE, etc. In some embodiments, a 5′-wing comprises 2-MOE modifications. In some embodiments, each 5′-wing sugar is 2′-MOE modified. In some embodiments, a 3′-wing comprises 2-OMe modifications. In some embodiments, each 3′-wing sugar is 2′-OMe modified.

In some embodiments, an internucleotidic linkage linking a wing nucleoside and a core nucleoside is considered a core internucleotidic linkage.

In some embodiments, a wing comprises one or more (e.g., 1, 2, 3, 4, 5, 6, 7, 8, 9, or 10) non-negatively charged internucleotidic linkages. In some embodiments, a non-negatively charged internucleotidic linkage is a neutral internucleotidic linkage. In some embodiments, as demonstrated herein, oligonucleotides that comprise wings comprising non-negatively charged internucleotidic linkages can deliver high activities and/or selectivities.

In some embodiments, a core sugar is a natural DNA sugar which comprises no substitution at the 2′ position (two —H at 2′-carbon). In some embodiments, each core sugar is a natural DNA sugar which comprises no substitution at the 2′ position (two —H at 2′-carbon).

In some embodiments, each wing and core is independently and optionally comprises a sugar comprising a nitrogen as described herein. In some embodiments, a 5′-wing comprises one or more sugar comprising nitrogen. In some embodiments, a 3′-wing comprises one or more sugar comprising nitrogen. In some embodiments, a core comprises one or more sugar comprising nitrogen.

As demonstrated herein, various oligonucleotides and compositions can provide various activities when incorporating sugars comprising nitrogen together with ribose/modified ribose sugars. Such sugars may also provide improved properties (e.g., charges, delivery, binding, selectivity, stability, etc.) over oligonucleotides comprising no sugars comprising nitrogen, and/or oligonucleotides comprising no ribose sugars (which can be independently modified or unmodified).

In some embodiments, a first wing (e.g., a 5′-wing) comprises one or more 2′-OR modifications, wherein R is optionally substituted C₁₋₄ aliphatic. In some embodiments, each sugar of a first wing comprises a 2′-OR modification. In some embodiments, 2′-OR is 2′-MOE. In some embodiments, each sugar of a first wing comprises 2′-MOE.

In some embodiments, a second wing (e.g., a 3′-wing) comprises one or more 2′-OR modifications, wherein R is optionally substituted C₁₋₄ aliphatic. In some embodiments, each sugar of a second wing comprises a 2′-OR modification. In some embodiments, 2′-OR is 2′-OMe. In some embodiments, each sugar of a second wing comprises 2′-OMe. In some embodiments, a second wing, e.g., a 3′-wing, does not share the same pattern of sugar modifications of a first wing, e.g., a 5′-wing. In some embodiments, a second wing, e.g., a 3′-wing, does not contain a sugar modification of a first wing, e.g., a 5′-wing. As appreciated by those skilled in the art, in some embodiments, a first wing can be a 3′-wing, and a second wing can be a 5′-wing.

In some embodiments, a core comprises 1-25, e.g., 1, 2, 3, 4, 5, 6, 7, 8, 9, 10, 11, 12, 13, 14, 15, 16, 17, 18, 19, 20, 21, 22, 23, 24, 25 or sugars that comprises no 2′-OR groups or are not bicyclic or polycyclic sugars. In some embodiments, a core comprises 1-25, e.g., 1, 2, 3, 4, 5, 6, 7, 8, 9, 10, 11, 12, 13, 14, 15, 16, 17, 18, 19, 20, 21, 22, 23, 24, 25 or sugars that comprises no 2′-OR groups. In some embodiments, a core comprises 1-25, e.g., 1, 2, 3, 4, 5, 6, 7, 8, 9, 10, 11, 12, 13, 14, 15, 16, 17, 18, 19, 20, 21, 22, 23, 24, 25 or sugars that comprises two 2′-H. In many embodiments, a core comprises no 2′-OR groups. In many embodiments, sugars in core regions have two 2′-H.

In some embodiments, certain sugar modifications, e.g., 2′-MOE, provide more stability under certain conditions than other sugar modifications, e.g., 2′-OMe. In some embodiments, a wing comprises 2′-MOE modifications. In some embodiments, each nucleoside unit of a wing comprising a pyrimidine base (e.g., C, U, T, etc.) comprises a 2′-MOE modification. In some embodiments, each sugar unit of a wing comprises a 2′-MOE modification. In some embodiments, each nucleoside unit of a wing comprising a purine base (e.g., A, G, etc.) comprises no 2′-MOE modification (e.g., each such nucleoside unit comprises 2′-OMe, or no 2′-modification, etc.). In some embodiments, each nucleoside unit of a wing comprising a purine base comprises a 2′-OMe modification. In some embodiments, each internucleotidic linkage at the 3′-position of a sugar unit comprising a 2′-MOE modification is a natural phosphate linkage.

In some embodiments, a wing comprises no 2′-MOE modifications. In some embodiments, a wing comprises 2′-OMe modifications. In some embodiments, each nucleoside unit of a wing independently comprises a 2′-OMe modification.

In some embodiments, a wing comprises a bicyclic sugar. In some embodiments, each wing independently comprises one or more bicyclic sugars.

In some embodiments, sugars are connected by internucleotidic linkages, in some embodiments, modified internucleotidic linkage. In some embodiments, an internucleotidic linkage does not contain a linkage phosphorus. In some embodiments, an internucleotidic linkage is -L-. In some embodiments, an internucleotidic linkage is —OP(O)(—C≡CH)—, —OP(O)(R)O— (e.g., R is —CH₃), 3′ —NHP(O)(OH)O—5′, 3′-OP(O)(CH₃)OCH₂—5′, 3′—CH₂C(O)NHCH₂—5′, 3′—SCH₂OCH₂—5′, 3′-OCH₂OCH₂—5′, 3′—CH₂NR′CH₂—5′, 3′—CH₂N(Me)OCH₂—5′, 3′—NHC(O)CH₂CH₂—5′, 3′—NR′C(O)CH₂CH₂-5′, 3′-CH₂CH₂NR′-5′, 3′-CH₂CH₂NH-5′, or 3′-OCH₂CH₂N(R′)-5′. In some embodiments, a 5′ carbon may be optionally substituted with ═O.

In some embodiments, a modified sugar is an optionally substituted pentose or hexose. In some embodiments, a modified sugar is an optionally substituted pentose. In some embodiments, a modified sugar is an optionally substituted hexose. In some embodiments, a modified sugar is an optionally substituted ribose or hexitol. In some embodiments, a modified sugar is an optionally substituted ribose. In some embodiments, a modified sugar is an optionally substituted hexitol.

In some embodiments, a sugar modification is 5′-vinyl (R or S), 5′-methyl (R or S), 2′-SH, 2′-F, 2′-OCH₃, 2′-OCH₂CH₃, 2′-OCH₂CH₂F or 2′-O(CH₂)₂₀CH₃. In some embodiments, a substituent at the 2′ position, e.g., a 2′-modification, is allyl, amino, azido, thio, O-allyl, O—C₁-C₁₀ alkyl, OCF₃, OCH₂F, O(CH₂)₂SCH₃, O(CH₂)₂; —O—N(Rₘ)(Rₙ), O—CH₂; —C(═O)—N(Rₘ)(Rₙ), and O—CH₂; —C(═O)—N(R₁)—(CH₂)₂; —N(Rₘ) (Rₙ), wherein each allyl, amino and alkyl is optionally substituted, and each of R₁, Rₘ and Rₙ is independently R′ as described in the present disclosure. In some embodiments, each of R₁, Rₘ and Rₙ is independently —H or optionally substituted C₁-C₁₀ alkyl.

In some embodiments, a sugar is a tetrahydropyran or THP sugar. In some embodiments, a modified nucleoside is tetrahydropyran nucleoside or THP nucleoside which is a nucleoside having a six-membered tetrahydropyran sugar substituted for a pentofuranosyl residue in typical natural nucleosides. THP sugars and/or nucleosides include those used in hexitol nucleic acid (HNA), anitol nucleic acid (ANA), mannitol nucleic acid (MNA) (e.g., Leumann, Bioorg. Med. Chem., 2002, 10, 841-854) or fluoro HNA (F-HNA).

In some embodiments, sugars comprise rings having more than 5 atoms and/or more than one heteroatom, e.g., morpholino sugars.

As those skilled in the art will appreciate, modifications of sugars, nucleobases, internucleotidic linkages, etc. can and are often utilized in combination in oligonucleotides, e.g., see various oligonucleotides in Table A1, A2, A3, and A4. For example, a combination of sugar modification and nucleobase modification is 2′-F (sugar) 5-methyl (nucleobase) modified nucleosides. In some embodiments, a combination is replacement of a ribosyl ring oxygen atom with S and substitution at the 2′-position.

In some embodiments, a 2′-modified sugar is a furanosyl sugar modified at the 2′ position. In some embodiments, a 2′-modification is halogen, —R′ (wherein R′ is not —H), —OR′ (wherein R′ is not —H), —SR′, —N(R′)₂, optionally substituted —CH₂; —CH═CH₂, optionally substituted alkenyl, or optionally substituted alkynyl. In some embodiments, a 2′-modifications is selected from —O[(CH₂)ₙO]ₘCH₃, —O(CH₂)ₙNH₂, —O(CH₂)ₙCH3, —O(CH₂)ₙF, —O(CH₂)ₙONH₂, —OCH₂C(═O)N(H)CH₃, and —O(CH₂)ₙON[(CH₂)ₙCH₃]₂, wherein each n and m is independently from 1 to about 10. In some embodiments, a 2′-modification is optionally substituted C₁-C₁₂ alkyl, optionally substituted alkenyl, optionally substituted alkynyl, optionally substituted alkaryl, optionally substituted aralkyl, optionally substituted -O-alkaryl, optionally substituted —O-aralkyl, —SH, —SCH₃, —OCN, —Cl, —Br, —CN, —F, —CF₃, —OCF₃, —SOCH₃, —SO₂CH₃, —ONO₂, —NO₂, —N₃, —NH₂, optionally substituted heterocycloalkyl, optionally substituted heterocycloalkaryl, optionally substituted aminoalkylamino, optionally substituted polyalkylamino, substituted silyl, a reporter group, an intercalator, a group for improving pharmacokinetic properties, a group for improving the pharmacodynamic properties, and other substituents. In some embodiments, a 2′-modification is a 2′-MOE modification.

In some embodiments, a 2′-modified or 2′-substituted sugar or nucleoside is a sugar or nucleoside comprising a substituent at the 2′ position of the sugar which is other than —H (typically not considered a substituent) or -OH. In some embodiments, a 2′-modified sugar is a bicyclic sugar comprising a bridge connecting two carbon atoms of the sugar ring one of which is the 2′ carbon. In some embodiments, a 2′-modification is non-bridging, e.g., allyl, amino, azido, thio, optionally substituted —O-allyl, optionally substituted —O—C₁-C₁₀ alkyl, —OCF₃, —O(CH₂)₂OCH₃, 2′-O(CH₂)₂SCH₃, —O(CH₂)₂ON(Rₘ)(Rₙ), or —OCH₂C(═O)N(Rₘ)(Rₙ), where each Rₘ and Rₙ is independently —H or optionally substituted C₁-C₁₀ alkyl.

In some embodiments, a sugar is the sugar of N-methanocarba, LNA, cMOE BNA, cEt BNA, α-L-LNA or related analogs, HNA, Me-ANA, MOE-ANA, Ara-FHNA, FHNA, R-6′-Me-FHNA, S-6′-Me-FHNA, ENA, or c-ANA. In some embodiments, a modified internucleotidic linkage is C3-amide (e.g., sugar that has the amide modification attached to the C3′, Mutisya et al. 2014 Nucleic Acids Res. 2014 Jun 1; 42(10): 6542-6551), formacetal, thioformacetal, MMI [e.g., methylene(methylimino), Peoc'h et al. 2006 Nucleosides and Nucleotides 16 (7-9)], a PMO (phosphorodiamidate linked morpholino) linkage (which connects two sugars), or a PNA (peptide nucleic acid) linkage.

In some embodiments, a sugar is one described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10450568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the sugars of each of which are incorporated herein by reference.

In some embodiments a modified sugar is one described in U.S. Pat. Nos. 5,658,873, 5,118,800, 5,393,878, 5,514,785, 5,627,053, 7,034,133,7084,125, 7,399,845, 5,319,080, 5,591,722, 5,597,909, 5,466,786, 6,268,490, 6,525,191, 5,519,134, 5,576,427, 6,794,499, 6,998,484, 7,053,207, 4,981,957, 5,359,044, 6,770,748, 7,427,672, 5,446,137, 6,670,461, 7,569,686, 7,741,457, 8,022,193, 8,030,467, 8,278,425, 5,610,300, 5,646,265, 8,278,426, 5,567,811, 5,700,920, 8,278,283, 5,639,873, 5,670,633, 8,314,227, US 2008/0039618, US 2009/0012281, WO 2021/030778, WO 2020/154344, WO 2020/154343, WO 2020/154342, WO 2020/165077, WO 2020/201406, WO 2020/216637, or WO 2020/252376.

Various additional sugars useful for preparing oligonucleotides or analogs thereof are known in the art and may be utilized in accordance with the present disclosure.

### Internucleotidic Linkages

Among other things, the present disclosure provides various internucleotidic linkages, including various modified internucleotidic linkages, either comprising phosphorus or not, that may be utilized together with other structural elements, e.g., various sugars as described herein, to provide oligonucleotides and compositions thereof.

As widely known by those skilled in the art, natural phosphate linkages are widely found in natural DNA and RNA molecules; they have the structure of —OP(O)(OH)—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S⁻)O—.

In some embodiments, an oligonucleotide comprises different types of internucleotidic phosphorus linkages. In some embodiments, a chirally controlled oligonucleotide comprises at least one natural phosphate linkage and at least one modified (non-natural) internucleotidic linkage. In some embodiments, an oligonucleotide comprises no natural phosphate linkages. In some embodiments, an oligonucleotide comprises at least one natural phosphate linkage and at least one phosphorothioate. In some embodiments, an oligonucleotide comprises at least one non-negatively charged internucleotidic linkage. In some embodiments, an oligonucleotide comprises at least one natural phosphate linkage and at least one non-negatively charged internucleotidic linkage. In some embodiments, an oligonucleotide comprises at least one phosphorothioate internucleotidic linkage and at least one non-negatively charged internucleotidic linkage. In some embodiments, an oligonucleotide comprises at least one phosphorothioate internucleotidic linkage, at least one natural phosphate linkage, and at least one non-negatively charged internucleotidic linkage. In some embodiments, oligonucleotides comprise one or more, e.g., 1-50, 1-40, 1-30, 1-20, 1-15, 1-10, 1, 2, 3, 4, 5, 6, 7, 8, 9, 10, 11, 12, 13, 14, 15, 16, 17, 18, 19, 20 or more non-negatively charged internucleotidic linkages. In some embodiments, a non-negatively charged internucleotidic linkage is not negatively charged in that at a given pH in an aqueous solution less than 50%, 40%, 40%, 30%, 20%, 10%, 5%, or 1% of the internucleotidic linkage exists in a negatively charged salt form. In some embodiments, a pH is about pH 7.4. In some embodiments, a pH is about 4-9. In some embodiments, the percentage is less than 10%. In some embodiments, the percentage is less than 5%. In some embodiments, the percentage is less than 1%. In some embodiments, an internucleotidic linkage is a non-negatively charged internucleotidic linkage in that the neutral form of the internucleotidic linkage has no pKa that is no more than about 1, 2, 3, 4, 5, 6, or 7 in water. In some embodiments, no pKa is 7 or less. In some embodiments, no pKa is 6 or less. In some embodiments, no pKa is 5 or less. In some embodiments, no pKa is 4 or less. In some embodiments, no pKa is 3 or less. In some embodiments, no pKa is 2 or less. In some embodiments, no pKa is 1 or less. In some embodiments, pKa of the neutral form of an internucleotidic linkage can be represented by pKa of the neutral form of a compound having the structure of CH3—the internucleotidic linkage-CH3. For example, pKa of

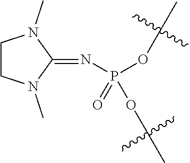 [SMILES: *OP(=O)(N=C1N(C)CCN1C)O*]

can be represented by pKa

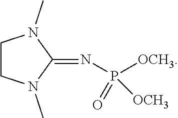

In some embodiments, a non-negatively charged internucleotidic linkage is a neutral internucleotidic linkage. In some embodiments, a non-negatively charged internucleotidic linkage is a positively-charged internucleotidic linkage. In some embodiments, a non-negatively charged internucleotidic linkage comprises a guanidine moiety. In some embodiments, a non-negatively charged internucleotidic linkage comprises a heteroaryl base moiety. In some embodiments, a non-negatively charged internucleotidic linkage comprises a triazole moiety. In some embodiments, a non-negatively charged internucleotidic linkage comprises an alkynyl moiety.

Without wishing to be bound by any particular theory, the present disclosure notes that a neutral internucleotidic linkage can be more hydrophobic than a phosphorothioate internucleotidic linkage (PS), which can be more hydrophobic than a natural phosphate linkage (PO). Typically, unlike a PS or PO, a neutral internucleotidic linkage bears less charge. Without wishing to be bound by any particular theory, the present disclosure notes that incorporation of one or more neutral internucleotidic linkages into an oligonucleotide may increase oligonucleotides' ability to be taken up by a cell and/or to escape from endosomes. Without wishing to be bound by any particular theory, the present disclosure notes that incorporation of one or more neutral internucleotidic linkages can be utilized to modulate melting temperature of duplexes formed between an oligonucleotide and its target nucleic acid. Without wishing to be bound by any particular theory, the present disclosure notes that incorporation of non-negatively charged internucleotidic linkages, e.g., neutral internucleotidic linkages, into oligonucleotides may be able to increase the oligonucleotides' ability to modulate levels, expressions and/or activities of target nucleic acids and/or products encoded thereby, e.g., through knock-down (e.g., by RNase H), exon skipping, etc. In some embodiments, a non-negatively charged internucleotidic linkage can improve the delivery and/or activity of an oligonucleotide.

In some embodiments, a linkage has the structure of or comprises —Y—P^{L}(—X—R^{L})—Z—, or a salt form thereof, wherein:

P^{L} is P, P(═W), P->B(-L^{L}-R^{L})₃, or P^{N};

W is O, N(-L^{L}-R^{L}), S or Se;

P^{N} is P═N—C(-L^{L}-R′)(=L^{N}-R′) or P═N-L^{L}-R^{L};

L^{N} is ═N-L^{L1}-, ═CH-L^{L1}—wherein CH is optionally substituted, or ═N⁺(R′)(Q⁻)-L^{L1}-;

Q⁻ is an anion;

each of X, Y and Z is independently —O—, —S—, -L^{L}-N(-L^{L}-R^{L})-L^{L}-, -L^{L}-N═C(-L^{L}-R^{L})-L^{L}-, or L^{L};

each R^{L} is independently -L^{L}-N(R′)₂, -L^{L}-R′, —N═C(-L^{L}-R′)₂, -L^{L}-N(R′)C(NR′)N(R′)₂, -L^{L}-N(R′)C(O)N(R′)₂, a carbohydrate, or one or more additional chemical moieties optionally connected through a linker;

each of L^{L1} and L^{L} is independently L;

-Cy^{IL}- is -Cy-;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—; —S—S—, —N(R′)—, —C(O)—, —C(S)—, —C(NR′)—, —C(NR′)N(R′)—, —N(R′)C(NR′)N(R′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—; —OP(O)(OR′)—, —OP(O)(SR′)—, —OP(O)(R′)—, —OP(O)(NR′)—, —OP(OR′)—, —OP(SR′)—, —OP(NR′)—, —OP(R′)—, —OP(OR′)[B(R′)₃]O—, and —[C(R′)₂C(R′)₂O]ₙ—, wherein n is 1-50, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)N(R)₂, —C(O)OR, or —S(O)₂R; each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, an internucleotidic linkage has the structure of —Y—P^{L}(—X—R^{L})—Z—, wherein each variable is independently as described herein. In some embodiments, an internucleotidic linkage has the structure of —O—P^{L}(—X—R^{L})—O—, wherein each variable is independently as described herein. In some embodiments, an internucleotidic linkage has the structure of —O—P(═W)(—X—R^{L})—O—, wherein each variable is independently as described herein. In some embodiments, an internucleotidic linkage has the structure of —O—P(═W)[—N(-L^{L}-R^{L})-R^{L}]—O—, wherein each variable is independently as described herein. In some embodiments, an internucleotidic linkage has the structure of 0 P(═W)(—NH—L^{L}-R^{L})—O—, wherein each variable is independently as described herein. In some embodiments, an internucleotidic linkage has the structure of —O—P(═W)[—N(R′)₂]—O—, wherein each variable is independently as described herein. In some embodiments, an internucleotidic linkage has the structure of —O—P(═W)(—NHR′)—O—, wherein each variable is independently as described herein. In some embodiments, an internucleotidic linkage has the structure of —O—P(═W)(—NHSO₂R)—O—, wherein each variable is independently as described herein. In some embodiments, R is methyl. In some embodiments, an internucleotidic linkage is —O—P(═O)(—NHSO₂CH₃)—O—. In some embodiments, an internucleotidic linkage has the structure of —O—P(═W)[—N═C(-L^{L}; —R′)₂]—O—, wherein each variable is independently as described herein. In some embodiments, an internucleotidic linkage has the structure of —O—P(═W)[—N═C[N(R′)₂]₂]—O—, wherein each variable is independently as described herein. In some embodiments, an internucleotidic linkage has the structure of —OP(═W)(—N═C(R″)₂)—O—, wherein each variable is independently as described herein. In some embodiments, an internucleotidic linkage has the structure of —OP(═W)(—N(R″)₂)—O—, wherein each variable is independently as described herein. In some embodiments, W is O. In some embodiments, W is S. In some embodiments, such an internucleotidic linkage is a non-negatively charged internucleotidic linkage. In some embodiments, such an internucleotidic linkage is a neutral internucleotidic linkage.

In some embodiments, an internucleotidic linkage has the structure of —P^{L}(—X—R^{L})—Z—, wherein each variable is independently as described herein. In some embodiments, an internucleotidic linkage has the structure of —P^{L}(—X—R^{L})—O—, wherein each variable is independently as described herein. In some embodiments, an internucleotidic linkage has the structure of P(═W)(—X—R^{L})—O—, wherein each variable is independently as described herein. In some embodiments, an internucleotidic linkage has the structure of wherein each variable is independently as described herein. In some embodiments, an internucleotidic linkage has the structure of —P(═W)(—NH-L^{L}-R^{L})—O—, wherein each variable is independently as described herein. In some embodiments, an internucleotidic linkage has the structure of —P(═W)[—N(R′)₂]—O—, wherein each variable is independently as described herein. In some embodiments, an internucleotidic linkage has the structure of —P(═W)(—NHR′)—O—, wherein each variable is independently as described herein. In some embodiments, an internucleotidic linkage has the structure of —P(═W)(—NHSO₂R)—O—, wherein each variable is independently as described herein. In some embodiments, R is methyl. In some embodiments, an internucleotidic linkage is —P(═O)(—NHSO₂CH₃)—O—. In some embodiments, an internucleotidic linkage has the structure of —P(═W)[—N═C(-L^{L}; —R′)₂]—O—, wherein each variable is independently as described herein. In some embodiments, an internucleotidic linkage has the structure of P(═W)[ N═C[N(R′)₂]₂]—O—, wherein each variable is independently as described herein. In some embodiments, an internucleotidic linkage has the structure of P(═W)(N═C(R″)₂)—O—, wherein each variable is independently as described herein. In some embodiments, an internucleotidic linkage has the structure of P(═W)(—N(R″)₂)—O—, wherein each variable is independently as described herein. In some embodiments, W is O. In some embodiments, W is S. In some embodiments, such an internucleotidic linkage is a non-negatively charged internucleotidic linkage. In some embodiments, such an internucleotidic linkage is a neutral internucleotidic linkage. In some embodiments, P of such an internucleotidic linkage is bonded to N of a sugar.

In some embodiments, a linkage is a phosphoryl guanidine internucleotidic linkage. In some embodiments, a linkage is a thio-phosphoryl guanidine internucleotidic linkage.

In some embodiments, one or more methylene units are optionally and independently replaced with a moiety as described herein. In some embodiments, L or L^{L} is or comprises —SO₂13 . In some embodiments, L or L^{L} is or comprises —SO₂N(R′)—. In some embodiments, L or L^{L} is or comprises —C(O)—. In some embodiments, L or L^{L} is or comprises —C(O)O—. In some embodiments, L or L^{L} is or comprises —C(O)N(R′)—. In some embodiments, L or L^{L} is or comprises —P(═W)(R′)—. In some embodiments, L or L^{L} is or comprises —P(═O)(R′)—. In some embodiments, L or L^{L} is or comprises —P(═S)(R′)—. In some embodiments, L or L^{L} is or comprises —P(R′)—. In some embodiments, L or L^{L} is or comprises —P(═W)(OR′)—. In some embodiments, L or L^{L} is or comprises —P(═O)(OR′)—. In some embodiments, L or L^{L} is or comprises —P(═S)(OR′)—. In some embodiments, L or L^{L} is or comprises —P(OR′)—.

In some embodiments, —X—R^{L} is —N(R′)SO₂R^{L}. In some embodiments, —X—R^{L} is —N(R′)C(O)R^{L}. In some embodiments, —X—R^{L} is —N(R′)P(═O)(R′)R^{L}.

In some embodiments, a linkage, e.g., a non-negatively charged internucleotidic linkage or neutral internucleotidic linkage, has the structure of or comprises —P(═W)(N═C(R″)₂)—, —P(═W)(—N(R′)SO₂R″)—, —P(═W)(—N(R′)C(O)R″)—, —P(═W)(—N(R″)₂)—, —P(═W)(—N(R′)P(O)(R″)₂)—, —OP(═W)(—N═C(R″)₂))O——OP(═W)(—N(R′)SO₂R″)O—, —OP(═W)(—N(R′)C(O)R″)O—, —OP(═W)(—N(R″)₂))O——OP(═W)(—N(R′)P(O)(R″)₂)O—, —P(═W)(—N═C(R″)₂)O——P(═W)(—N(R′)SO₂R″)O—, —P(═W)(—N(R′)C(O)R″)O—, —P(═W)(—N(R″)₂)O—, or —P(═W)(—N(R′)P(O)(R″)₂)O—, or a salt form thereof, wherein:

W is O or S;

each R^{M1} is independently R′, —OR′, —P(═W)(R′)₂, or —N(R′)₂;

each R′ is independently —R, —C(O)R, —C(O)N(R)₂, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, W is O. In some embodiments, an internucleotidic linkage has the structure of P(═O)(N═C(R″)₂)—, —P(═O)(—N(R′)SO₂R″)—, —P(═O)(—N(R′)C(O)R″)—, —P(═O)(—N(R″)₂) , P(═O)(N(R′)P(O)(R″)₂) , OP(═O)(N═C(R″)₂)O—, —OP(═O)(—N(R′)SO₂R″)O—, —OP(═O)(—N(R′)C(O)R″)O—, —OP(═O)(—N(R″)₂)O—, —OP(═O)(—N(R′)P(O)(R″)₂))O—, —P(═O)(—N═C(R″)₂)O—, —P(═O)(—N(R′)SO₂R″)O—, —P(═O)(—N(R′)C(O)R″)O—, —P(═O)(—N(R″)₂)O—, or —P(═O)(—N(R′)P(O)(R″)₂)O—, or a salt form thereof. In some embodiments, an internucleotidic linkage has the structure of —P(═O)(—N═C(R″)₂)— —P(═O)(—N(R″)₂)—, —OP(═O)(—N═C(R″)₂)—O——OP(═O)(—N(R″)₂)—O—, —P(═O)(—N═C(R″)₂)—O— or —P(═O)(—N(R″)₂)—O— or a salt form thereof. In some embodiments, an internucleotidic linkage has the structure of —OP(═O)(N═C(R″)₂)—O— or —OP(═O)(—N(R″)₂)—O—, or a salt form thereof. In some embodiments, an internucleotidic linkage has the structure of —OP(═O)(N═C(R″)₂)—O—, or a salt form thereof. In some embodiments, an internucleotidic linkage has the structure of —OP(═O)(—N(R″)₂)—O—, or a salt form thereof. In some embodiments, an internucleotidic linkage has the structure of —OP(═O)(—N(R′)SO₂R″)O—, or a salt form thereof. In some embodiments, an internucleotidic linkage has the structure of —OP(═O)(—N(R′)C(O)R″)O—, or a salt form thereof. In some embodiments, an internucleotidic linkage has the structure of —OP(═O)(—N(R′)P(O)(R″)₂)O—, or a salt form thereof. In some embodiments, a internucleotidic linkage is n001.

In some embodiments, W is S. In some embodiments, an internucleotidic linkage has the structure of —P(═S)(—N═C(R″)₂)—, —P(═S)(—N(R′)SO₂R″)—, —P(═S)(—N(R′)C(O)R″)—, —P(═S)(—N(R″)₂)—, —P(═S)(—N(R′)P(O)(R″)₂)—, —OP(═S)(—N═C(R″)₂)O—, —OP(═S)(—N(R′)SO₂R″)O—, —OP(═S)(—N(R′)C(O)R″)O—, —OP(═S)(—N(R″)₂)O—, —OP(═S)(—N(R′)P(O)(R″)₂))O—, —P(═S)(—N═C(R″)₂)O—, —P(═S)(—N(R′)SO₂R″)O—, —P(═S)(—N(R′)C(O)R″)O—, —P(═S)(—N(R″)₂)O—, or —P(═S)(—N(R′)P(O)(R″)₂)O—, or a salt form thereof. In some embodiments, an internucleotidic linkage has the structure of —P(═S)(—N═C(R″)₂)— —P(═S)(—N(R″)₂)—, —OP(═S)(—N═C(R″)₂)—O—, —OP(═S)(—N(R″)₂)—O—, —P(═S)(—N═C(R″)₂)—O— or —P(═S)(—N(R″)₂)—O— or a salt form thereof. In some embodiments, an internucleotidic linkage has the structure of —OP(═S)(—N═C(R″)₂)—O— or —OP(═S)(—N(R″)₂)—O—, or a salt form thereof. In some embodiments, an internucleotidic linkage has the structure of —OP(═S)(N═C(R″)₂)—O—, or a salt form thereof. In some embodiments, an internucleotidic linkage has the structure of —OP(═S)(—N(R″)₂)—O—, or a salt form thereof. In some embodiments, an internucleotidic linkage has the structure of —OP(═S)(—N(R′)SO₂R″)O—, or a salt form thereof. In some embodiments, an internucleotidic linkage has the structure of —OP(═S)(—N(R′)C(O)R″)O—, or a salt form thereof. In some embodiments, an internucleotidic linkage has the structure of —OP(═S)(—N(R′)P(O)(R″)₂)O—, or a salt form thereof. In some embodiments, a internucleotidic linkage is *n001.

In some embodiments, an internucleotidic linkage has the structure of —P(═O)(—N(R′)SO₂R″)—, wherein R^{M1} is as described herein. In some embodiments, an internucleotidic linkage has the structure of P(═S)(N(R′)SO₂R″)—, wherein R^{M1} is as described herein. In some embodiments, an internucleotidic linkage has the structure of —P(═O)(—N(R′)SO₂R″)O—, wherein R^{M1} is as described herein. In some embodiments, an internucleotidic linkage has the structure of —P(═S)(—N(R′)SO₂R″)O—, wherein R^{M1} is as described herein. In some embodiments, an internucleotidic linkage has the structure of —OP(═O)(—N(R′)SO₂R″)O—, wherein R^{M1} is as described herein. In some embodiments, an internucleotidic linkage has the structure of —OP(═S)(—N(R′)SO₂R″)O—, wherein R^{M1} is as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —SO₂R″, is R′ as described herein. In some embodiments, an internucleotidic linkage has the structure of —P(═O)(—NHSO₂R″)—, wherein R^{M1} is as described herein. In some embodiments, an internucleotidic linkage has the structure of P(═S)(NHSO₂R″)—, wherein R^{M1} is as described herein. In some embodiments, an internucleotidic linkage has the structure of —P(═O)(—NHSO₂R″)—, wherein R^{M1} is as described herein. In some embodiments, an internucleotidic linkage has the structure of —P(═S)(—NHSO₂R″)—, wherein R^{M1} is as described herein. In some embodiments, an internucleotidic linkage has the structure of —OP(═O)(—NHSO₂R″)—, wherein R^{M1} is as described herein. In some embodiments, an internucleotidic linkage has the structure of —OP(═S)(—NHSO₂R″)—, wherein R^{M1} is as described herein. In some embodiments, —X—R^{L} is —N(R′)SO₂R^{L}, wherein each of R′ and R^{L} is independently as described herein. In some embodiments, R^{L} is R″. In some embodiments, R^{L} is R′. In some embodiments, —X—R^{L} is —N(R′)SO₂R″, wherein R′ is as described herein. In some embodiments, —X—R^{L} is —N(R′)SO₂R′, wherein R′ is as described herein. In some embodiments, —X—R^{L} is -NHSO₂R′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in —SO₂R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is optionally substituted methyl. In some embodiments, —X—R^{L} is —NHSO₂CH3. In some embodiments, R is —CF₃. In some embodiments, R is methyl. In some embodiments, R is optionally substituted ethyl. In some embodiments, R is ethyl. In some embodiments, R is —CH₂CHF2. In some embodiments, R is —CH₂CH₂OCH₃. In some embodiments, R is optionally substituted propyl. In some embodiments, R is optionally substituted butyl. In some embodiments, R is n-butyl. In some embodiments, R is —(CH₂)₆NH₂. In some embodiments, R is an optionally substituted linear C2-20 aliphatic. In some embodiments, R is optionally substituted linear C2-20 alkyl. In some embodiments, R is linear C2-20 alkyl. In some embodiments, R is optionally substituted C1, C2, C3, C4, Cs, C6, C7, Cs, C9, Cu), C₁₂, C₁₂, C13, C₁₋₄, C15, C16, C17, C18, C19, or C20 aliphatic. In some embodiments, R is optionally substituted C1, C2, C3, C4, C5, C6, C7, C8, C9, C10, C11, C₁₂, C13, C₁₋₄, C15, C16, C17, C18, C19, or C20 alkyl. In some embodiments, R is optionally substituted linear C₁, C₂, C₃, C₄, C₅, C₆, C₇, C₈, C₉, C₁₀, C₁₁, Cᵤ, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, Cᵤ, C₁₃, C₁₄, C₁₅, C₁₆, C₁₇, C₁₈, C19, or C₂₀ alkyl. In some embodiments, R is optionally substituted phenyl. In some embodiments, R is phenyl. In some embodiments, R is p-methylphenyl. In some embodiments, R is 4-dimethylaminophenyl. In some embodiments, R is 3-pyridinyl. In some embodiments, R is

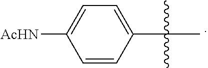 [SMILES: *c1ccc(NC(C)=O)cc1]

In some embodiments, R is

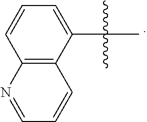 [SMILES: *c1cccc2ncccc12]

In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R is isopropyl. In some embodiments, R^{M1} is —N(R′)₂. In some embodiments, R^{M1} is —N(CH₃)₂. In some embodiments, R″, e.g., in —SO₂R″, is —OR′, wherein R′ is as described herein. In some embodiments, R′ is R as described herein. In some embodiments, R^{M1} is —OCH₃. In some embodiments, a linkage is —OP(═O)(—NHSO₂R)O—, wherein R is as described herein. In some embodiments, R is optionally substituted linear alkyl as described herein. In some embodiments, R is linear alkyl as described herein. In some embodiments, a linkage is —OP(═O)(—NHSO₂CH₃)O—. In some embodiments, a linkage is —OP(═O)(—NHSO₂CH₂CH₃)O—. In some embodiments, a linkage is —OP(═O)(—NHSO₂CH₂CH₂OCH₃)O—. In some embodiments, a linkage is —OP(═O)(—NHSO₂CH₂Ph)O—. In some embodiments, a linkage is —OP(═O)(—NHSO₂CH₂CHF₂)O—. In some embodiments, a linkage is —OP(═O)(—NHSO₂(4-methylphenyl))O—. In some embodiments, —X—R^{L is}

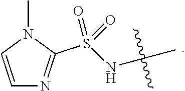 [SMILES: *NS(=O)(=O)c1nccn1C]

In some embodiments, a linkage is —OP(═O)(—X—R^{L})O—, wherein —X—R^{L} is

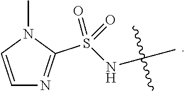 [SMILES: *NS(=O)(=O)c1nccn1C]

In some embodiments, a linkage is —OP(═O)(—NHSO₂CH(CH₃)₂)O—. In some embodiments, a linkage is —OP(═O)(—NHSO₂N(CH₃)₂)O—.

In some embodiments, an internucleotidic linkage has the structure of —P(═O)(—N(R′)C(O)R″)—, wherein R^{M1} is as described herein. In some embodiments, an internucleotidic linkage has the structure of —P(═S)(—N(R′)C(O)R″)—, wherein R^{M1} is as described herein. In some embodiments, an internucleotidic linkage has the structure of —P(═O)(—N(R′)C(O)R″)O—, wherein R^{M1} is as described herein. In some embodiments, an internucleotidic linkage has the structure of —P(═S)(—N(R′)C(O)R″)O—, wherein R^{M1} is as described herein. In some embodiments, an internucleotidic linkage has the structure of —OP(═O)(—N(R′)C(O)R″)O—, wherein R^{M1} is as described herein. In some embodiments, an internucleotidic linkage has the structure of —OP(═S)(—N(R′)C(O)R″)O—, wherein R^{M1} is as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —C(O)R″, is R′ as described herein. In some embodiments, an internucleotidic linkage has the structure of —P(═O)(—NHC(O)R″)—, wherein R^{M1} is as described herein. In some embodiments, an internucleotidic linkage has the structure of —P(═S)(—NHC(O)R″)—, wherein R^{M1} is as described herein. In some embodiments, an internucleotidic linkage has the structure of —P(═O)(—NHC(O)R″)—, wherein R^{M1} is as described herein. In some embodiments, an internucleotidic linkage has the structure of —P(═S)(—NHC(O)R″)—, wherein R^{M1} is as described herein. In some embodiments, an internucleotidic linkage has the structure of —OP(═O)(—NHC(O)R″)—, wherein R^{M1} is as described herein. In some embodiments, an internucleotidic linkage has the structure of —OP(═S)(—NHC(O)R″)—, wherein R^{M1} is as described herein. In some embodiments, —X—R^{L} is —N(R′)COR^{L}, wherein R^{L} is as described herein. In some embodiments, —X—R^{L} is —N(R′)COR″, wherein R^{M1} is as described herein. In some embodiments, —X—R^{L} is —N(R′)COR′, wherein R′ is as described herein. In some embodiments, —X—R^{L} is -NHCOR′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in —C(O)R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, —X—R^{L} is —NHC(O)CH3. In some embodiments, R is optionally substituted methyl. In some embodiments, R is -CF₃. In some embodiments, R is optionally substituted ethyl. In some embodiments, R is ethyl. In some embodiments, R is —CH₂CHF2. In some embodiments, R is —CH₂CH₂OCH₃. In some embodiments, R is optionally substituted C₁₋₂₀ (e.g., C₁₋₆, C₂₋₆, C₃₋₆, C₁₋₁₀, C₂₋₁₀, C₃₋₁₀, C2-20, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C₂₋₆, C₃₋₆, C₁₋₁₀, C₂₋₁₀, C₃₋₁₀, C2-20, C₃₋₂₀, C₁₀₋₂₀, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C2-20 aliphatic. In some embodiments, R is optionally substituted linear C2-20 alkyl. In some embodiments, R is linear C2-20 alkyl. In some embodiments, R is optionally substituted C₁, C₂, C₃, C₄, C₅, C₆, C₇, C₈, C₉, C₁₀, C₁₁, C₁₂, C₁₃, C₁₋₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, Cᵤ, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R^{L} is —(CH₂)₅NH₂. In some embodiments, R^{L} is

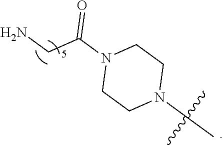

In some embodiments, R^{L} is

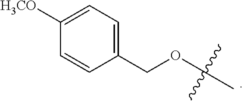 [SMILES: *OCc1ccc(OC)cc1]

In some embodiments, R^{M1} is —N(R′)₂. In some embodiments, R^{M1} is —N(CH₃)₂. In some embodiments, is —N(R′)CON(R^{L})₂, wherein each of R′ and R^{L} is independently as described herein. In some embodiments, —X—R^{L} is —NHCON(R^{L})₂, wherein R^{L} is as described herein. In some embodiments, two R′ or two R^{L} are taken together with the nitrogen atom to which they are attached to form a ring as described herein, e.g., optionally substituted

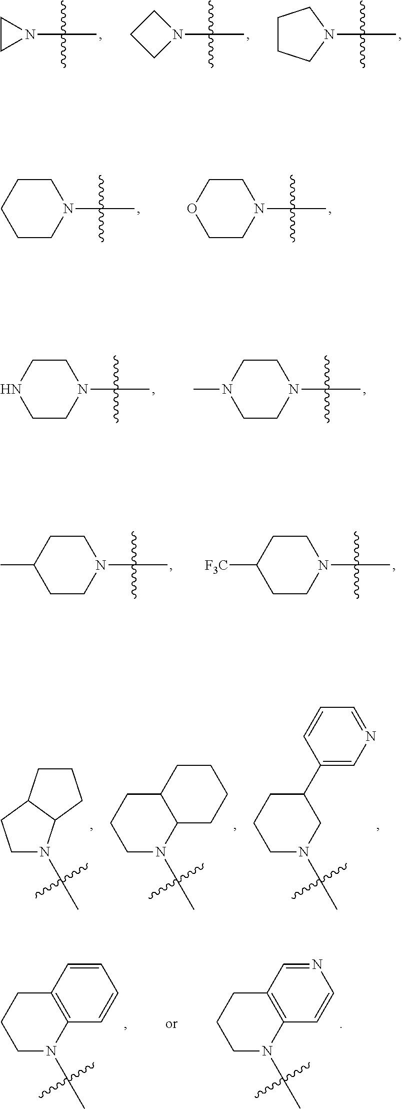

In some embodiments, R″, e.g., in —C(O)R″, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R^{M1} is —OCH₃. In some embodiments, is —N(R′)C(O)₀₁V⁻, wherein each of R′ and R^{L} is independently as described herein. In some embodiments, R is

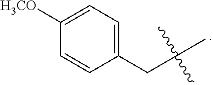 [SMILES: *Cc1ccc(OC)cc1]

In some embodiments, —X—R^{L} is —NHC(O)OCH₃. In some embodiments, —X—R^{L} is —NHC(O)N(CH₃)₂. In some embodiments, a linkage is —OP(O)(NHC(O)CH₃)O—. In some embodiments, a linkage is —OP(O)(NHC(O)OCH₃)O—. In some embodiments, a linkage is —OP(O)(NHC(O)(p-methylphenyl))O—. In some embodiments, a linkage is —OP(O)(NHC(O)N(CH₃)₂)O—. In some embodiments, —X—R^{L} is —N(R′)R^{L}, wherein each of R′ and R^{L} is independently as described herein. In some embodiments, —X—R^{L} is —N(R′)R^{L}, wherein each of R′ and R^{L} is independently not hydrogen. In some embodiments, —X—R^{L} is —NHR^{L}, wherein R^{L} is as described herein. In some embodiments, R^{L} is not hydrogen. In some embodiments, R^{L} is optionally substituted aryl or heteroaryl. In some embodiments, R^{L} is optionally substituted aryl. In some embodiments, R^{L} is optionally substituted phenyl. In some embodiments, —X—R^{L} is —N(R′)₂, wherein each R′ is independently as described herein. In some embodiments, —X—R^{L} is —NHR′, wherein R′ is as described herein. In some embodiments, —X—R^{L} is —NHR, wherein R is as described herein. In some embodiments, —X—R^{L} is R^{L}, wherein R^{L} is as described herein. In some embodiments, R^{L} is —N(R′)₂, wherein each R′ is independently as described herein. In some embodiments, R^{L} is —NHR′, wherein R′ is as described herein. In some embodiments, R^{L} is —NHR, wherein R is as described herein. In some embodiments, R^{L} is —N(R′)₂, wherein each R′ is independently as described herein. In some embodiments, none of R′ in —N(R′)₂ is hydrogen. In some embodiments, R^{L} is —N(R′)₂, wherein each R′ is independently C₁₋₆ aliphatic. In some embodiments, R^{L} is -L-R′, wherein each of L and R′ is independently as described herein. In some embodiments, R^{L} is -L-R, wherein each of L and R is independently as described herein. In some embodiments, R^{L} is —N(R′)-Cy-N(R′)—R′. In some embodiments, R^{L} is —N(R′)-Cy-C(O)—R′. In some embodiments, R^{L} is —N(R′)-Cy-O—R′. In some embodiments, R^{L} is —N(R′)-Cy-SO₂; —R′. In some embodiments, R^{L} is —N(R′)-Cy-SO₂; —N(R′)₂. In some embodiments, R^{L} is —N(R′)-Cy-C(O)—N(R′)₂. In some embodiments, R^{L} is —N(R′)-Cy-OP(O)(R″)₂. In some embodiments, -Cy- is an optionally substituted bivalent aryl group. In some embodiments, -Cy-is optionally substituted phenylene. In some embodiments, -Cy- is optionally substituted 1,4-phenylene. In some embodiments, -Cy- is 1,4-phenylene. In some embodiments, R^{L} is —N(CH₃)₂. In some embodiments, R^{L} is —N(i-Pr)₂. In some

embodiments, R^{L} is

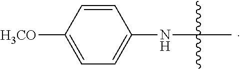 [SMILES: *Nc1ccc(OC)cc1]

In some embodiments, R^{L} is

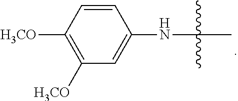 [SMILES: *Nc1ccc(OC)c(OC)c1]

In some

embodiments, R^{L} is

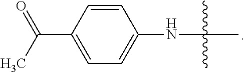 [SMILES: *Nc1ccc(C(C)=O)cc1]

In some embodiments, R^{L} is

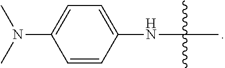 [SMILES: *Nc1ccc(N(C)C)cc1]

In some embodiments, R^{L} is

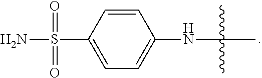 [SMILES: *Nc1ccc(S(N)(=O)=O)cc1]

In some embodiments, R^{L} is

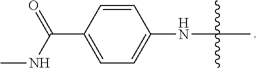 [SMILES: *Nc1ccc(C(=O)NC)cc1]

In some embodiments, R^{L} is

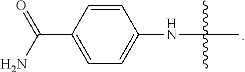 [SMILES: *Nc1ccc(C(N)=O)cc1]

In some embodiments, R^{L} is

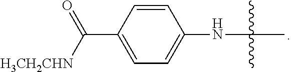

In some embodiments, R^{L} is

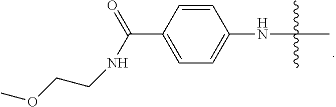 [SMILES: *Nc1ccc(C(=O)NCCOC)cc1]

In some embodiments, R^{L} is

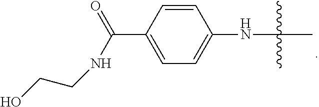 [SMILES: *Nc1ccc(C(=O)NCCO)cc1]

In some embodiments, R^{L} is

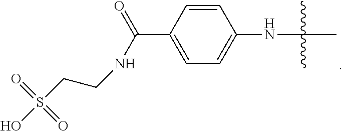 [SMILES: *Nc1ccc(C(=O)NCCS(=O)(=O)O)cc1]

In some embodiments, R^{L} is

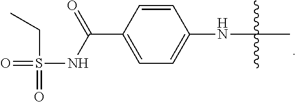 [SMILES: *Nc1ccc(C(=O)NS(=O)(=O)CC)cc1]

In some embodiments, R^{L} is

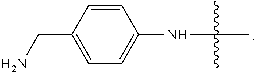 [SMILES: *Nc1ccc(CN)cc1]

In some embodiments, R^{L} is

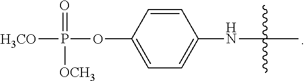 [SMILES: *Nc1ccc(OP(=O)(OC)OC)cc1]

In some embodiments, R^{L} is

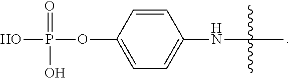 [SMILES: *Nc1ccc(OP(=O)(O)O)cc1]

In some embodiments, R^{L} is

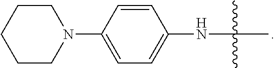 [SMILES: *Nc1ccc(N2CCCCC2)cc1]

In some embodiments, R^{L} is

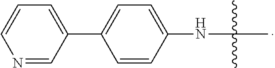 [SMILES: *Nc1ccc(-c2cccnc2)cc1]

In some embodiments, R^{L} is

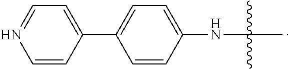 [SMILES: *Nc1ccc(-c2ccncc2)cc1]

In some embodiments, R^{L} is

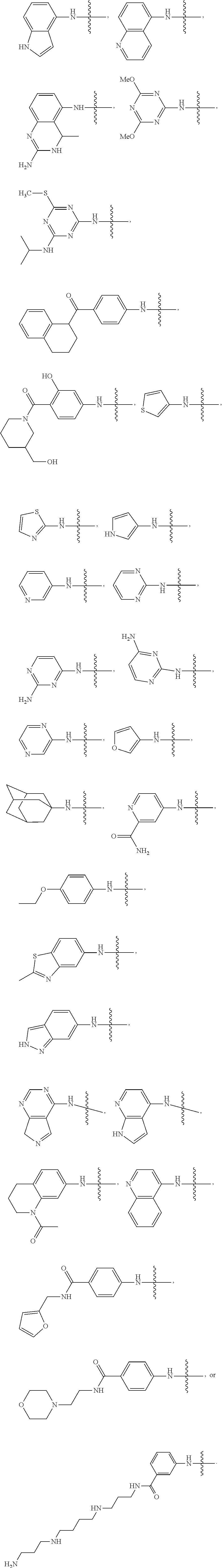

In some embodiments, —X—R^{L} is —N(R′)—C(O)-Cy-R^{L}. In some embodiments, —X—R^{L} is R^{L}. In some embodiments, R^{L} is —N(R′)—C(O)-Cy-O—R′. In some embodiments, R^{L} is —N(R′)—C(O)-Cy-R′. In some embodiments, R^{L} is —N(R′)—C(O)-Cy-C(O)—R′. In some embodiments, R^{L} is —N(R′)—C(O)-Cy-N(R′)₂. In some embodiments, R^{L} is —N(R′)—C(O)-Cy-SO₂—N(R′)₂. In some embodiments, R^{L} is —N(R′)—C(O)-Cy-C(O)—N(R′)₂. In some embodiments, R^{L} is —N(R′)—C(O)-Cy-C(O)—N(R′)—SO₂; —R′. In some embodiments, R′ is R as described herein. In some embodiments, R^{L} is

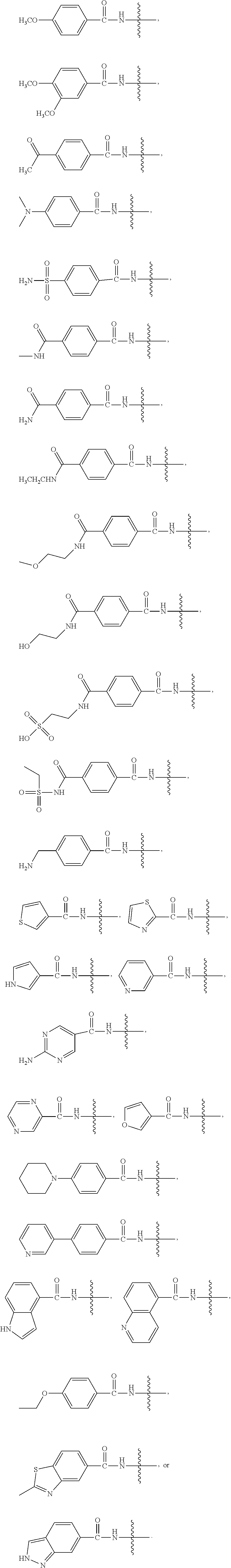

As described herein, in some embodiments, one or more methylene units of L, or a variable which comprises or is L, are independently replaced with —O—, —N(R′)—, —C(O)—, —C(O)N(R′)—, —SO₂—-, —SO₂N(R′)—, or -Cy-. In some embodiments, a methylene unit is replaced with -Cy-. In some embodiments, -Cy- is an optionally substituted bivalent aryl group. In some embodiments, -Cy- is optionally substituted phenylene. In some embodiments, -Cy- is optionally substituted 1,4-phenylene. In some embodiments, -Cy- is an optionally substituted bivalent 5-20 (e.g. 5, 6, 7, 8, 9, 10, 11, 12, 13, 14, 15, 16, 17, 18, 19, or 20) membered heteroaryl group having 1-10 (e.g., 1, 2, 3, 4, 5, 6, 7, 8, 9, or 10) heteroatoms. In some embodiments, -Cy- is monocyclic. In some embodiments, -Cy- is bicyclic. In some embodiments, -Cy- is polycyclic. In some embodiments, each monocyclic unit in -Cy- is independently 3-10 (e.g., 3, 4, 5, 6, 7, 8, 9, or 10) membered, and is independently saturated, partially saturated, or aromatic. In some embodiments, -Cy- is an optionally substituted 3-20 (e.g., 3, 4, 5, 6, 7, 8, 9, 10, 11, 12, 13, 14, 15, 16, 17, 18, 19, or 20) membered monocyclic, bicyclic or polycyclic aliphatic group. In some embodiments, -Cy- is an optionally substituted 3-20 (e.g., 3, 4, 5, 6, 7, 8, 9, 10, 11, 12, 13, 14, 15, 16, 17, 18, 19, or 20) membered monocyclic, bicyclic or polycyclic heteroaliphatic group having 1-10 (e.g., 1, 2, 3, 4, 5, 6, 7, 8, 9, or 10) heteroatoms.

In some embodiments, an internucleotidic linkage has the structure of —P(═O)(—N(R′)P(O)(R″)₂)—, wherein each R^{M1} is independently as described herein. In some embodiments, an internucleotidic linkage has the structure of P(═S)(N(R′)P(O)(R″)₂)—, wherein each R^{M1} is independently as described herein. In some embodiments, an internucleotidic linkage has the structure of —P(═O)(—N(R′)P(O)(R″)₂)—, wherein each R^{M1} is independently as described herein. In some embodiments, an internucleotidic linkage has the structure of —P(═S)(—N(R′)P(O)(R″)₂)—, wherein each R^{M1} is independently as described herein. In some embodiments, an internucleotidic linkage has the structure of —OP(═O)(—N(R′)P(O)(R″)₂)—, wherein each R^{M1} is independently as described herein. In some embodiments, an internucleotidic linkage has the structure of —OP(═S)(—N(R′)P(O)(R″)₂)—, wherein each R^{M1} is independently as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —P(O)(R″)₂, is R′ as described herein. In some embodiments, an internucleotidic linkage has the structure of —P(═O)(—NHP(O)(R″)₂)—, wherein each R^{M1} is independently as described herein. In some embodiments, an internucleotidic linkage has the structure of —P(═S)(—NHP(O)(R″)₂)—, wherein each R^{M1} is independently as described herein. In some embodiments, an internucleotidic linkage has the structure of —P(═O)(—NHP(O)(R″)₂)—, wherein each R^{M1} is independently as described herein. In some embodiments, an internucleotidic linkage has the structure of —P(═S)(—NHP(O)(R″)₂)—, wherein each R^{M1} is independently as described herein. In some embodiments, an internucleotidic linkage has the structure of —OP(═O)(—NHP(O)(R″)₂)—, wherein each R^{M1} is independently as described herein. In some embodiments, an internucleotidic linkage has the structure of —OP(═S)(—NHP(O)(R″)₂)—, wherein each R^{M1} is independently as described herein. In some embodiments, an occurrence of R″, e.g., in —P(O)(R″)₂, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, R is optionally substituted methyl. In some embodiments, R is —CF3. In some embodiments, R is optionally substituted ethyl. In some embodiments, R is ethyl. In some embodiments, R is —CH₂CHF2. In some embodiments, R is —CH₂CH₂OCH₃. In some embodiments, R is optionally substituted C_{1,20} (e.g., C₁₋₆, C₂₋₆, C₃₋₆, C₁₋₁₀, C₂₋₁₀, C₃₋₁₀, C2-20, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C₂₋₆, C₃₋₆, C₁₋₁₀, C₂₋₁₀, C₃₋₁₀, C2-20, C₃₋₂₀, C₁₀₋₂₀, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C2-20 aliphatic. In some embodiments, R is optionally substituted linear C2-20 alkyl. In some embodiments, R is linear C2-20 alkyl. In some embodiments, R is isopropyl. In some embodiments, R is optionally substituted C₁, C₂, C₃, C₄, Cs, C₆, C₇, Cs, C₉, Cio, Cii, C₁₂, C₁₃, C₁₋₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₋₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, Cᵤ, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, Cᵤ, C₁₃, C₁₄, C₁₅, C₁₆, C₁₇, C₁₈, C₁₉, or C₂₀ alkyl. In some embodiments, each R^{M1} is independently R as described herein, for example, in some embodiments, each R^{M1} is methyl. In some embodiments, R^{M1} is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, an occurrence of R^{M1} is —N(R′)₂. In some embodiments, R^{M1} is —N(CH₃)₂. In some embodiments, an occurrence of R″, e.g., in —P(O)(R″)₂, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R^{M1} is —OCH₃. In some embodiments, each R^{M1} is —OR′ as described herein. In some embodiments, each R^{M1} is —OCH₃. In some embodiments, each R^{M1} is —OH. In some embodiments, a linkage is —OP(O)(NHP(O)(OH)₂)O—. In some embodiments, a linkage is —OP(O)(NHP(O)(OCH₃)₂)O—. In some embodiments, a linkage is —OP(O)(NHP(O)(CH₃)₂)O—.

In some embodiments, —N(R″)₂ is —N(R′)₂. In some embodiments, —N(R″)₂ is —NHR. In some embodiments, —N(R″)₂ is —NHC(O)R. In some embodiments, —N(R″)₂ is —NHC(O)OR. In some embodiments, —N(R″)₂ is —NHS(O)₂R.

In some embodiments, an internucleotidic linkage is a phosphoryl guanidine internucleotidic linkage. In some embodiments, an internucleotidic linkage comprises —X-12L as described herein. In some embodiments, —X—R^{L} is —N═C(-L^{L}-R^{L})₂. In some embodiments, —X—R^{L} is —N═C[N(R^{L})₂]₂. In some embodiments, —X—R^{L} is —N═C[NR′R^{L}]₂. In some embodiments, —X—R^{L} is —N═C[N(R′)₂]₂. In some embodiments, —X—R^{L} is —N═C[N(R^{L})₂](CHR^{L1}R)wherein each of R^{L1} and R^{L2} is independently as described herein. In some embodiments, —X—R^{L} is —N═C(NR′R^{L})(CHR^{L1}R)wherein each of R^{L1} and R^{L2} is independently as described herein. In some embodiments, —X—R^{L} is N═C(NR′R^{L})(CR′R^{L1}R^{L2}) wherein each of R^{L1} and R^{L2} is independently as described herein. In some embodiments, —X—R^{L} is —N═C[N(R′)₂](CHR′R^{L2}). In some embodiments, —X —R^{L} is N═C[N(R^{L})₂](R^{L}). In some embodiments, —X—R^{L} is N═C(NR′R^{L})(R^{L}). In some embodiments, —X—R^{L} is —N═C(NR′R^{L})(R′). In some embodiments, —X—R^{L} is —N═C[N(R′)₂](R′). In some embodiments, —X—R^{L} is —N═C(NR′R^{L2}), wherein each R^{L1} and R^{L2} is independently R^{L}, and each R′ and R^{L} is independently as described herein. In some embodiments, —X—R^{L} is —N═C(NR′R)wherein variable is independently as described herein. In some embodiments, —X—R^{L} is —N═C(NR′R^{L1})(CHR′R^{L2}), wherein variable is independently as described herein. In some embodiments, —X—R^{L} is —N═C(NR′R^{L1})(R′), wherein variable is independently as described herein. In some embodiments, each R′ is independently R. In some embodiments, R is optionally substituted C₁₋₆ aliphatic. In some embodiments, R is methyl. In some embodiments, —X—R^{L} is

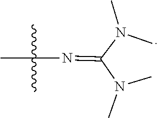 [SMILES: *N=C(N(C)C)N(C)C]

In some embodiments, two groups selected from R′, R^{L}, R^{L1}, R^{R2}, etc. (in some embodiments, on the same atom (e.g., —N(R′)₂, or —NR′R^{L}, or —N(R^{L})², wherein R′ and R^{L} can independently be R as described herein), etc.), or on different atoms (e.g., the two R′ in —N═C(NR′R^{L})(CR′R^{L1}R^{L2}) or —N═C(NR′R^{L1})(NR′R^{L2}) can also be two other variables that can be R, e.g., R^{L}, R^{L1}, R^{L2}, etc.)) are independently R and are taken together with their intervening atoms to form a ring as described herein. In some embodiments, two of R, R′, R^{L}, R^{L1}, or R^{L2} on the same atom, e.g., of —N(R′)₂, —N(R^{L})₂, —NR′R^{L}, —NR′R^{L1}, —NR′R^{L2}, —CR′R^{L1}R^{L2}, etc., are taken together to form a ring as described herein. In some embodiments, two R′, R^{L}, R^{L1}, or R^{L2} on two different atoms, e.g., the two R′ in —N═C(NR′R^{L})(CR′R^{L1}R^{L2}) —N═C(NR′R^{L1})(NR′R^{L2}), etc. are taken together to form a ring as described herein. In some embodiments, a formed ring is an optionally substituted 3-20 (e.g., 3-15, 3-12, 3-10, 3-9, 3-8, 3-7, 3-6, 4-15, 4-12, 4-10, 4-9, 4-8, 4-7, 4-6, 5-15, 5-12, 5-10, 5-9, 5-8, 5-7, 5-6, 1, 2, 3, 4, 5, 6, 7, 8, 9, 10, 11, 12, 13, 14, 15, 16, 17, 18, 19, 20, etc.) monocyclic, bicyclic or tricyclic ring having 0-5 additional heteroatoms. In some embodiments, a formed ring is monocyclic as described herein. In some embodiments, a formed ring is an optionally substituted 5-10 membered monocyclic ring. In some embodiments, a formed ring is bicyclic. In some embodiments, a formed ring is polycyclic. In some embodiments, two groups that are or can be R (e.g., the two R′ in —N═C(NR′R′R^{L})(CR′R^{L1}R^{L2}) or —N═C(NR′R^{L1})(NR′R^{L2}), the two R′ in —N═C(NR′R^{L})(CR′R^{L1}R^{L2}), —N═C(NR′R^{L1})(NR′R^{L2}), etc.) are taken together to form an optionally substituted bivalent hydrocarbon chain, e.g., an optionally substituted C₁₋₂₀ aliphatic chain, optionally substituted —(CH₂)n— wherein n is 1-20 (e.g., 1, 2, 3, 4, 5, 6, 7, 8, 9, 10, 11, 12, 13, 14, 15, 16, 17, 18, 19, or 20). In some embodiments, a hydrocarbon chain is saturated. In some embodiments, a hydrocarbon chain is partially unsaturated. In some embodiments, a hydrocarbon chain is unsaturated. In some embodiments, two groups that are or can be R (e.g., the two R′ in —N═C(NR′R^{L})(CR′R^{L1}R^{L2}) or —N═C(NR′R^{L1})(NR′R^{L2}); the two R′ in —N═C(NR′R^{L})(CR′R^{L1}R^{L2}); —N═C(NR′R^{L1})(NR′R^{L2}); etc.) are taken together to form an optionally substituted bivalent heteroaliphatic chain, e.g., an optionally substituted C₁₋₂₀ heteroaliphatic chain having 1-10 heteroatoms. In some embodiments, a heteroaliphatic chain is saturated. In some embodiments, a heteroaliphatic chain is partially unsaturated. In some embodiments, a heteroaliphatic chain is unsaturated. In some embodiments, a chain is optionally substituted —(CH₂)—. In some embodiments, a chain is optionally substituted —(CH₂)₂—. In some embodiments, a chain is optionally substituted —(CH₂)—. In some embodiments, a chain is optionally substituted —(CH₂)₂-. In some embodiments, a chain is optionally substituted —(CH₂)₃—. In some embodiments, a chain is optionally substituted —(CH₂)₄—. In some embodiments, a chain is optionally substituted —(CH₂)₅—. In some embodiments, a chain is optionally substituted —(CH₂)₆—. In some embodiments, a chain is optionally substituted —CH=CH—. In some embodiments, a chain is optionally substituted

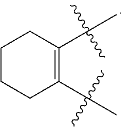 [SMILES: *C1=C(*)CCCC1]

In some embodiments, a chain is optionally substituted

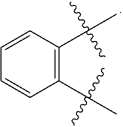 [SMILES: *c1ccccc1*]

In some embodiments, a chain is optionally substituted

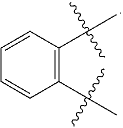 [SMILES: *c1ccccc1*]

In some embodiments, a chain is optionally substituted

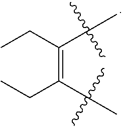 [SMILES: */C(CC)=C(\*)CC]

In some embodiments, a chain is optionally substituted

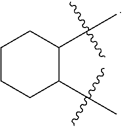 [SMILES: *C1CCCCC1*]

In some embodiments, a chain is optionally substituted

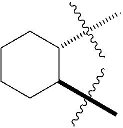 [SMILES: *[C@H]1CCCC[C@@H]1*]

In some embodiments, a chain is optionally substituted

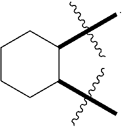 [SMILES: *[C@@H]1CCCC[C@@H]1*]

In some embodiments, a chain is optionally substituted

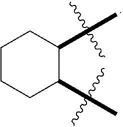 [SMILES: *[C@@H]1CCCC[C@@H]1*]

In some embodiments, a chain is optionally substituted

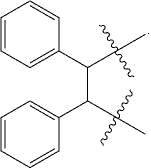 [SMILES: *C(c1ccccc1)C(*)c1ccccc1]

In some embodiments, two of R, R′, R^{L}, R^{L1}, R^{L2}, etc. on different atoms are taken together to form a ring as described herein. For examples, in some embodiments, —X—R^{L} is

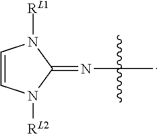

In some embodiments, —X—R^{L} is

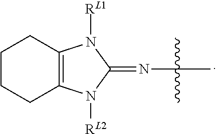

In some embodiments, —X—R^{L} is

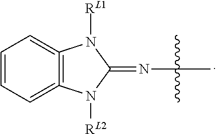

In some embodiments, —X—R^{L} is

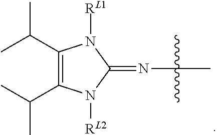

In some embodiments, —X—R^{L} is

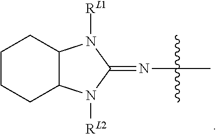

In some embodiments, —X—R^{L} is

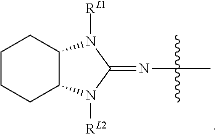

In some embodiments, —X—R^{L} is

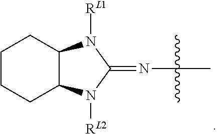

In some embodiments, —X—R^{L} is

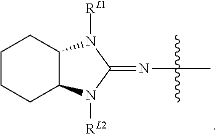

In some embodiments, —X—R^{L} is

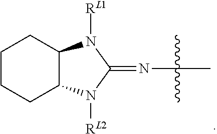

In some embodiments, —X—R^{L} is

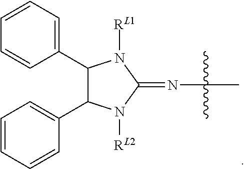

In some embodiments, —N(R′)₂, —N(R)₂, —N(R^{L})₂, —NR′R^{L}, —NR′R^{L1}, —NR′R^{L2}, —NR^{L1}R^{L2}, etc. is a formed ring. In some embodiments, a ring is optionally substituted

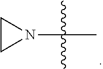 [SMILES: *N1CC1]

In some embodiments, a ring is optionally substituted

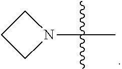 [SMILES: *N1CCC1]

In some embodiments, a ring is optionally substituted

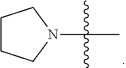 [SMILES: *N1CCCC1]

In some embodiments, a ring is optionally substituted

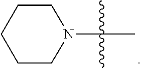 [SMILES: *N1CCCCC1]

In some embodiments, a ring is optionally substituted

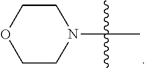 [SMILES: *N1CCOCC1]

In some embodiments, a ring is optionally substituted

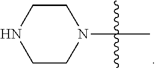 [SMILES: *N1CCNCC1]

In some embodiments, a ring is optionally substituted

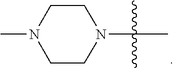 [SMILES: *N1CCN(C)CC1]

In some embodiments, a ring is optionally substituted

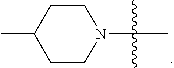 [SMILES: *N1CCC(C)CC1]

In some embodiments, a ring is optionally substituted

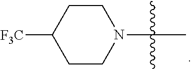 [SMILES: *N1CCC(C(F)(F)F)CC1]

In some embodiments, a ring is optionally substituted

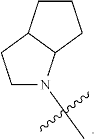 [SMILES: *N1CCC2CCCC21]

In some embodiments, a ring is optionally substituted

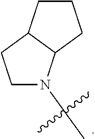 [SMILES: *N1CCC2CCCC21]

In some embodiments, a ring is optionally substituted

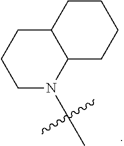 [SMILES: *N1CCCC2CCCCC21]

In some embodiments, a ring is optionally substituted

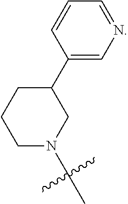 [SMILES: *N1CCCC(c2cccnc2)C1]

In some embodiments, a ring is optionally substituted

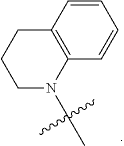 [SMILES: *N1CCCc2ccccc21]

In some embodiments, a ring is optionally substituted

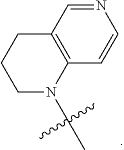 [SMILES: *N1CCCc2cnccc21]

In some embodiments, R^{L1} and R^{L2} are the same. In some embodiments, R^{L1} and R^{L2} are different. In some embodiments, each of R^{L1} and R^{L2} is independently R^{L} as described herein, e.g., below.

In some embodiments, R^{L} is optionally substituted C₁₋₃₀ aliphatic. In some embodiments, R^{L} is optionally substituted C₁₋₃₀ alkyl. In some embodiments, R^{L} is linear. In some embodiments, R^{L} is optionally substituted linear C₁₋₃₀ alkyl. In some embodiments, R^{L} is optionally substituted C₁₋₆ alkyl. In some embodiments, R^{L} is methyl. In some embodiments, R^{L} is ethyl. In some embodiments, R^{L} is n-propyl. In some embodiments, R^{L} is isopropyl. In some embodiments, R^{L} is n-butyl. In some embodiments, R^{L} is tert-butyl. In some embodiments, R^{L} is (E)-CH₂—CH═CH—CH₂—CH₃. In some embodiments, R^{L} is (Z)—CH₂—CH═CH—CH₂—CH₃. In some embodiments, R^{L} is

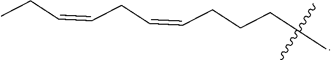 [SMILES: *CCC/C=C\C/C=C\CC]

In some embodiments, R^{L} is

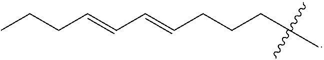 [SMILES: *CCC/C=C/C=C/CCC]

In some embodiments, R^{L} is CH₃(CH₂)₂C≡CC≡C(CH₂)₃—. In some embodiments, R^{L} is CH₃(CH₂)₅C≡C—. In some embodiments, R^{L} optionally substituted aryl. In some embodiments, R^{L} is optionally substituted phenyl. In some embodiments, R^{L} is phenyl substituted with one or more halogen. In some embodiments, R^{L} is phenyl optionally substituted with halogen, —N(R′), or —N(R′)C(O)R′. In some embodiments, R^{L} is phenyl optionally substituted with —Cl, —Br, —F, —N(Me)₂, or —NHCOCH₃. In some embodiments, R^{L} is -L^{L}-R′, wherein L^{L} is an optionally substituted C₁₋₂₀ saturated, partially unsaturated or unsaturated hydrocarbon chain. In some embodiments, such a hydrocarbon chain is linear. In some embodiments, such a hydrocarbon chain is unsubstituted. In some embodiments, L^{L} is (E)-CH₂—CH═CH—. In some embodiments, L^{L} is —CH₂—CC≡CH₂—. In some embodiments, L^{L} is —(CH₂)₃—. In some embodiments, L^{L} is —(CH₂)₄-. In some embodiments, L^{L} is —(CH₂)₃—, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R′ is optionally substituted aryl as described herein. In some embodiments, R′ is optionally substituted phenyl. In some embodiments, R′ is phenyl. In some embodiments, R′ is optionally substituted heteroaryl as described herein. In some embodiments, R′ is 2′-pyridinyl. In some embodiments, R′ is 3′-pyridinyl. In some embodiments, R^{L} is

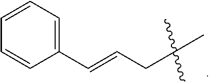 [SMILES: *C/C=C/c1ccccc1]

In some embodiments, R^{L} is

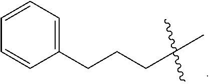 [SMILES: *CCCc1ccccc1]

In some embodiments, R^{L} is

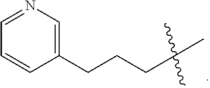 [SMILES: *CCCc1cccnc1]

In some embodiments, R^{L} is -L^{L}-N(R′)₂, wherein each variable is independently as described herein. In some embodiments, each R′ is independently C₁₋₆ aliphatic as described herein. In some embodiments, —N(R′)₂ is —N(CH₃)₂. In some embodiments, —N(R′)₂ is —NH₂. In some embodiments, R^{L} is —(CH₂)ₙ—N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙ—CH₂CH₂—N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is

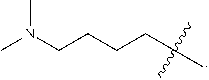 [SMILES: *CCCCN(C)C]

In some embodiments, R^{L} is

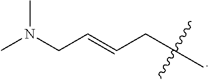 [SMILES: *C/C=C/CN(C)C]

In some embodiments, R^{L} is

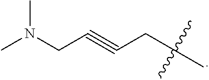 [SMILES: *CC#CCN(C)C]

In some embodiments, R^{L} is —(CH₂)ₙ—NH₂. In some embodiments, R^{L} is —(CH₂CH₂O)ₙ—CH₂CH₂—NH₂. In some embodiments, R^{L} is —(CH₂CH₂O)ₙ—CH₂CH₂—R′, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙ—CH₂CH₂CH₃, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙ—CH₂CH₂OH, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is or comprises a carbohydrate moiety, e.g., GalNAc. In some embodiments, R^{L} is -L^{L}-GalNAc. In some embodiments, R^{L} is

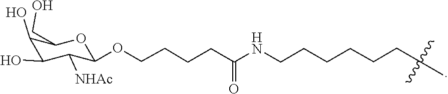 [SMILES: *CCCCCCNC(=O)CCCCOC1OC(CO)C(O)C(O)C1NC(C)=O]

In some embodiments, one or more methylene units of L^{L} are independently replaced with -Cy- (e.g., optionally substituted 1,4-phenylene, a 3-30 membered bivalent optionally substituted monocyclic, bicyclic, or polycyclic cycloaliphatic ring, etc.), —O—, —N(R′)—(e.g., —NH), —C(O)—, —C(O)N(R′)— (e.g., —C(O)NH—), —C(NR′)— (e.g., —C(NH)—), —N(R′)C(O)(N(R′)— (e.g., —NHC(O)NH—), —N(R′)C(NR′)(N(R′)— (e.g., —NHC(NH)NH—), —(CH₂CH₂O)ₙ—, etc. For example, in some embodiments, R^{L} is

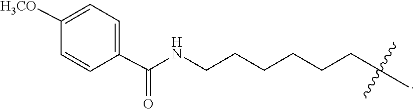 [SMILES: *CCCCCCNC(=O)c1ccc(OC)cc1]

In some embodiments, R^{L} is

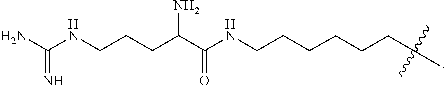 [SMILES: *CCCCCCNC(=O)C(N)CCCNC(=N)N]

In some embodiments, R^{L} is

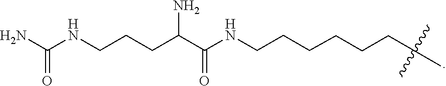 [SMILES: *CCCCCCNC(=O)C(N)CCCNC(N)=O]

In some embodiments, R^{L} is

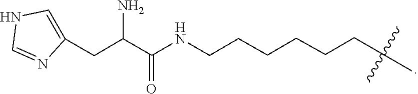 [SMILES: *CCCCCCNC(=O)C(N)Cc1c[nH]cn1]

In some embodiments, R^{L} is

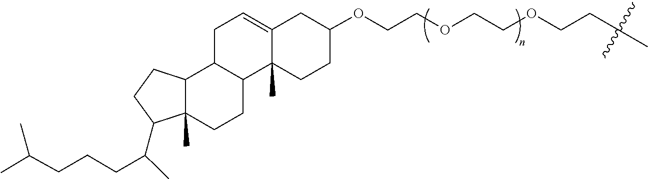

wherein n is 0-20. In some embodiments, R^{L} is or comprises one or more additional chemical moieties (e.g., carbohydrate moieties, GalNAc moieties, etc.) optionally substituted connected through a linker (which can be bivalent or polyvalent). For example, in some embodiments, R^{L} is

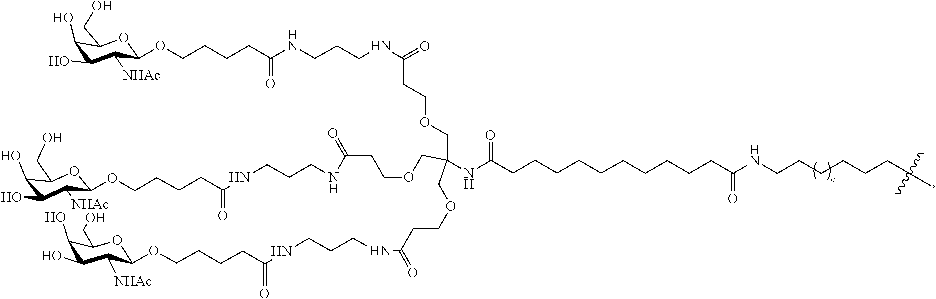

wherein n is 0-20. In some embodiments, R^{L} is

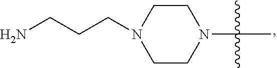

wherein n is 0-20. In some embodiments, R^{L} is R′ as described herein. As described herein, many variable can independently be R′. In some embodiments, R′ is R as described herein. As described herein, various variables can independently be R. In some embodiments, R is optionally substituted C₁₋₆ aliphatic. In some embodiments, R is optionally substituted C₁₋₆ alkyl. In some embodiments, R is methyl. In some embodiments, R is optionally substituted cycloaliphatic. In some embodiments, R is optionally substituted cycloalkyl. In some embodiments, R is optionally substituted aryl. In some embodiments, R is optionally substituted phenyl. In some embodiments, R is optionally substituted heteroaryl. In some embodiments, R is optionally substituted heterocyclyl. In some embodiments, R is optionally substituted C₁₋₂₀ heterocyclyl having 1-5 heteroatoms, e.g., one of which is nitrogen. In some embodiments, R is optionally substituted

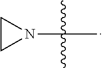 [SMILES: *N1CC1]

In some embodiments, R is optionally substituted

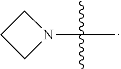 [SMILES: *N1CCC1]

In some embodiments, R is optionally substituted

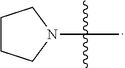 [SMILES: *N1CCCC1]

In some embodiments, R is optionally substituted

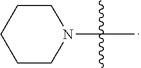 [SMILES: *N1CCCCC1]

In some embodiments, R is optionally substituted

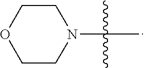 [SMILES: *N1CCOCC1]

In some embodiments, R is optionally substituted

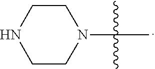 [SMILES: *N1CCNCC1]

In some embodiments, R is optionally substituted

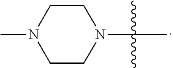 [SMILES: *N1CCN(C)CC1]

In some embodiments, R is optionally substituted

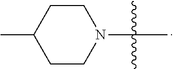 [SMILES: *N1CCC(C)CC1]

In some embodiments, R is optionally substituted

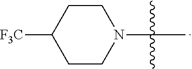 [SMILES: *N1CCC(C(F)(F)F)CC1]

In some embodiments, R is optionally substituted

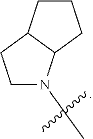 [SMILES: *N1CCC2CCCC21]

In some embodiments, R is optionally substituted

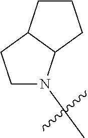 [SMILES: *N1CCC2CCCC21]

In some embodiments, R is optionally substituted

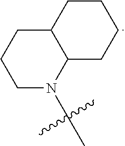 [SMILES: *N1CCCC2CCCCC21]

In some embodiments, R is optionally substituted

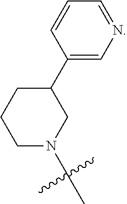 [SMILES: *N1CCCC(c2cccnc2)C1]

In some embodiments, R is optionally substituted

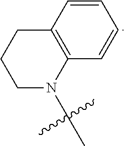 [SMILES: *N1CCCc2ccccc21]

In some embodiments, R is optionally substituted

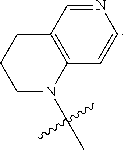 [SMILES: *N1CCCc2cnccc21]

In some embodiments, —X—R^{L} is

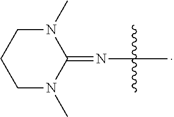 [SMILES: *N=C1N(C)CCCN1C]

In some embodiments, —X—R^{L} is

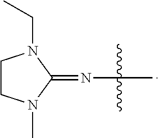 [SMILES: *N=C1N(C)CCN1CC]

In some embodiments, —X—R^{L} is

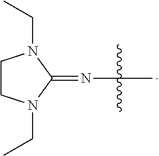 [SMILES: *N=C1N(CC)CCN1CC]

In some embodiments, —X—R^{L} is

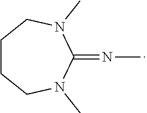

In some embodiments, —X—R^{L} is

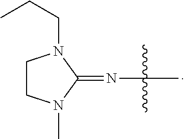 [SMILES: *N=C1N(C)CCN1CCC]

In some embodiments, —X—R^{L} is

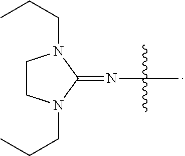 [SMILES: *N=C1N(CCC)CCN1CCC]

In some embodiments, —X—R^{L} is

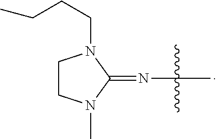 [SMILES: *N=C1N(C)CCN1CCCC]

In some embodiments, —X—R^{L} is

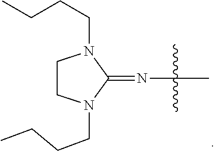 [SMILES: *N=C1N(CCCC)CCN1CCCC]

In some embodiments, —X—R^{L} is

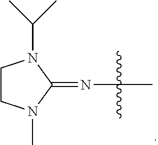 [SMILES: *N=C1N(C)CCN1C(C)C]

In some embodiments, —X—R^{L} is

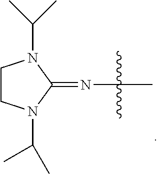 [SMILES: *N=C1N(C(C)C)CCN1C(C)C]

In some embodiments, —X—R^{L} is

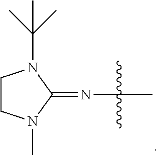 [SMILES: *N=C1N(C)CCN1C(C)(C)C]

In some embodiments, —X—R^{L} is

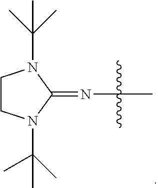 [SMILES: *N=C1N(C(C)(C)C)CCN1C(C)(C)C]

In some embodiments, —X—R^{L} is

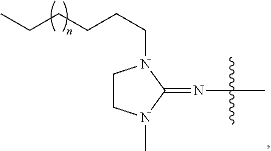

wherein n is 1-20. In some embodiments, —X—R^{L} is

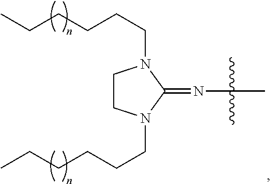

wherein n is 1-20. In some embodiments, —X—R^{L} is selected from:

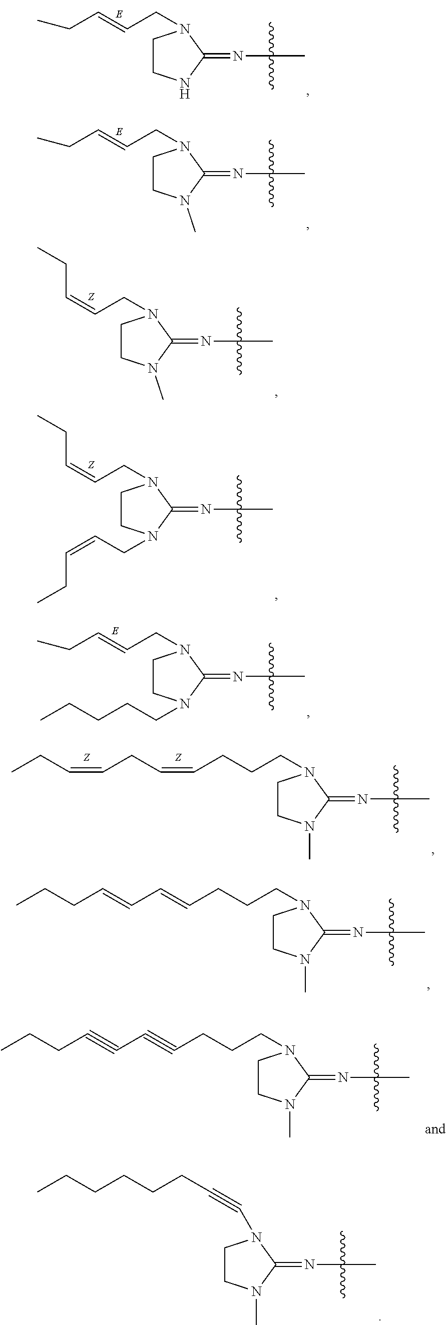

In some embodiments, —X—R^{L} is

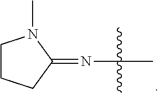 [SMILES: *N=C1CCCN1C]

In some embodiments, —X—R^{L} is

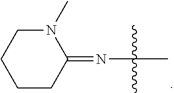 [SMILES: *N=C1CCCCN1C]

In some embodiments, —X—R^{L} is

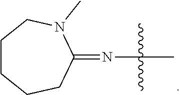 [SMILES: *N=C1CCCCCN1C]

In some embodiments, R^{L} is R^{M1} as described herein. In some embodiments, R^{L} is R as described herein.

In some embodiments, R^{M1} or R^{L} is or comprises an additional chemical moiety. In some embodiments, R^{M1} or R^{L} is or comprises an additional chemical moiety, wherein the additional chemical moiety is or comprises a carbohydrate moiety. In some embodiments, R^{M1} or R^{L} is or comprises a GalNAc. In some embodiments, R^{L} or R^{M1} is replaced with, or is utilized to connect to, an additional chemical moiety.

In some embodiments, X is —O—. In some embodiments, X is —5; —. In some embodiments, X is -L^{L}-N(-L^{L}-R^{L})-L^{L}-. In some embodiments, X is —N(-L^{L}-R^{L})-L^{L}-. In some embodiments, X is -L^{L}-N(-L^{L}-R^{L})—. In some embodiments, X is —N(-L^{L}-R^{L})-. In some embodiments, X is -L^{L}-N═C(-L^{L}-R^{L})-L^{L}-. In some embodiments, X is —N═C(-L^{L}-R^{L})-L^{L}-. In some embodiments, X is -L^{L}-N═C(-L^{L}-R^{L}) In some embodiments, X is —N═C(-L^{L}-R^{L})-. In some embodiments, X is L^{L}. In some embodiments, X is a covalent bond.

In some embodiments, Y is a covalent bond. In some embodiments, Y is —O—. In some embodiments, Y is —N(R′)—. In some embodiments, Z is a covalent bond. In some embodiments, Z is —O—. In some embodiments, Z is —N(R′)—. In some embodiments, R′ is R. In some embodiments, R is —H. In some embodiments, R is optionally substituted C₁₋₆ aliphatic. In some embodiments, R is methyl. In some embodiments, R is ethyl. In some embodiments, R is propyl. In some embodiments, R is optionally substituted phenyl. In some embodiments, R is phenyl.

As described herein, various variables in structures in the present disclosure can be or comprise R. Suitable embodiments for R are described extensively in the present disclosure. As appreciated by those skilled in the art, R embodiments described for a variable that can be R may also be applicable to another variable that can be R. Similarly, embodiments described for a component/moiety (e.g., L) for a variable may also be applicable to other variables that can be or comprise the component/moiety.

In some embodiments, R^{M1} is R′. In some embodiments, R^{M1} is —N(R′)₂.

In some embodiments, —X—R^{L} is —SH. In some embodiments, —X—R^{L} is —OH.

In some embodiments, —X—R^{L} is —N(R′)₂. In some embodiments, each R′ is independently optionally substituted C₁₋₆ aliphatic. In some embodiments, each R′ is independently methyl.

In some embodiments, a non-negatively charged internucleotidic linkage has the structure of —OP(═O)(—N═C((N(R′)₂)₂—O—. In some embodiments, a R′ group of one N(R′)₂ is R, a R′ group of the other N(R′)₂ is R, and the two R groups are taken together with their intervening atoms to form an optionally substituted ring, e.g., a 5-membered ring as in n001. In some embodiments, each R′ is independently R, wherein each R is independently optionally substituted C₁₋₆ aliphatic.

In some embodiments, —X—R^{L} is N═C((N(R′₂. In some embodiments, —X —R^{L} is N═C(— L^{L1}-L^{L2}-L^{L3}-R′)₂, wherein each L^{L1}, L^{L2} and L^{L3} is independently L″, wherein each L″ is independently a covalent bond, or a bivalent, optionally substituted, linear or branched group selected from a C₁₋₁₀ aliphatic group and a C₁₋₁₀ heteroaliphatic group having 1-5 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′))O——OP(O)(SR′))O——OP(O)(R′)O—, —OP(O)(NR′)O—, —OP(OR′)O—, —OP(SR′)O—, —OP(NR′))O——OP(R′)O—, or —OP(OR′)[B(R′)₃]O—, and one or more nitrogen or carbon atoms are optionally and independently replaced with Cy^{L}. In some embodiments, L^{L2} is -Cy-. In some embodiments, L^{u} is a covalent bond. In some embodiments, L^{L3} is a covalent bond. In some embodiments, —X—R^{L} is —N═C(-L^{L1}-Cy-L^{L3}-R′)₂. In some embodiments, —X—R^{L is}

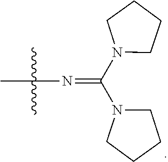 [SMILES: *N=C(N1CCCC1)N1CCCC1]

In some embodiments, —X—R^{L} is

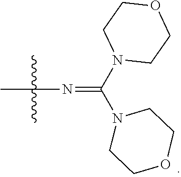 [SMILES: *N=C(N1CCOCC1)N1CCOCC1]

In some embodiments, —X—R^{L} is

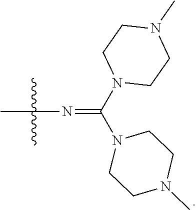 [SMILES: *N=C(N1CCN(C)CC1)N1CCN(C)CC1]

In some embodiments, —X—R^{L} is

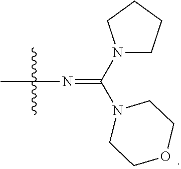 [SMILES: *N=C(N1CCCC1)N1CCOCC1]

In some embodiments, —X—R^{L} is

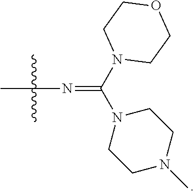 [SMILES: *N=C(N1CCOCC1)N1CCN(C)CC1]

In some embodiments, —X—R^{L} is

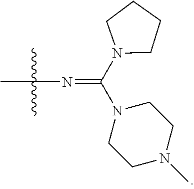 [SMILES: *N=C(N1CCCC1)N1CCN(C)CC1]

In some embodiments, as utilized in the present disclosure, L is covalent bond. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)O—, —OP(O)(NR′)—, —OP(OR′)—, —OP(SR′)—, —OP(NR′)—, —OP(R′)—, or —OP(OR′)[B(R′)₃]—, and one or more nitrogen or carbon atoms are optionally and independently replaced with Cy^{L}. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from —CEC— —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)—, —OP(O)(R′)—, —OP(O)(NR′)—, —OP(OR′)—, —OP(SR′)—, —OP(NR′)O—, —OP(R′)—, or —OP(OR′)[B(R′)₃]O—and one or more nitrogen or carbon atoms are optionally and independently replaced with Cy^{L}. In some embodiments, L is a bivalent, optionally substituted, linear or branched group selected from a C₁₋₁₀ aliphatic group and a C₁₋₁₀ heteroaliphatic group having 1-10 heteroatoms, wherein one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂-, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)O—, —OP(SR′)—, —OP(NR′)—, —OP(R′)—, or —OP(OR′)[B(R′)₃]O—and one or more nitrogen or carbon atoms are optionally and independently replaced with Cy^{L}. In some embodiments, one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, or —C(O)O—.

In some embodiments, an internucleotidic linkage is a phosphoryl guanidine internucleotidic linkage. In some embodiments, —X—R^{L} is —N═C[N(R′)₂]₂. In some embodiments, each R′ is independently R. In some embodiments, R is optionally substituted C₁₋₆ aliphatic. In some embodiments, R is methyl. In some embodiments, —X—R^{L} is

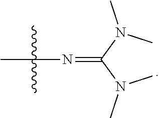 [SMILES: *N=C(N(C)C)N(C)C]

In some embodiments, one R′ on a nitrogen atom is taken with a R′ on the other nitrogen to form a ring as described herein.

In some embodiments, —X—R^{L} is

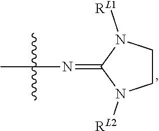

wherein R¹ and R² are independently R′. In some embodiments, —X—R^{L} is

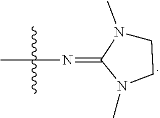 [SMILES: *N=C1N(C)CCN1C]

In some embodiments, —X—R^{L} is

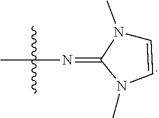 [SMILES: *N=c1n(C)ccn1C]

In some embodiments, two R′ on the same nitrogen are taken together to form a ring as described herein. In some embodiments, —X—R^{L} is

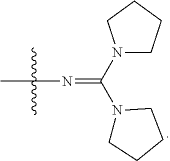 [SMILES: *N=C(N1CCCC1)N1CCCC1]

In some embodiments, —X—R^{L} is

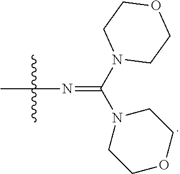 [SMILES: *N=C(N1CCOCC1)N1CCOCC1]

In some embodiments, —X—R^{L} is

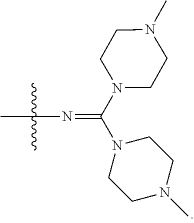 [SMILES: *N=C(N1CCN(C)CC1)N1CCN(C)CC1]

In some embodiments, —X—R^{L} is

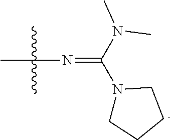 [SMILES: *N=C(N(C)C)N1CCCC1]

In some embodiments, —X—R^{L} is

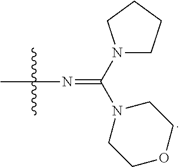 [SMILES: *N=C(N1CCCC1)N1CCOCC1]

In some embodiments, —X—R^{L} is

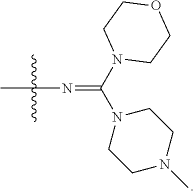 [SMILES: *N=C(N1CCOCC1)N1CCN(C)CC1]

In some embodiments, —X—R^{L} is

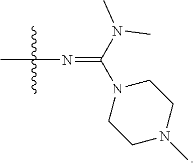 [SMILES: *N=C(N(C)C)N1CCN(C)CC1]

In some embodiments, —X—R^{L} is

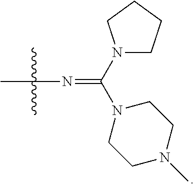 [SMILES: *N=C(N1CCCC1)N1CCN(C)CC1]

In some embodiments, —X—R^{L} is

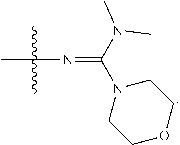 [SMILES: *N=C(N(C)C)N1CCOCC1]

In some embodiments, —X—R^{L} is

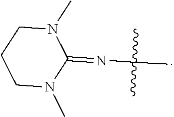 [SMILES: *N=C1N(C)CCCN1C]

In some embodiments, —X—R^{L} is

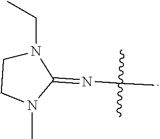 [SMILES: *N=C1N(C)CCN1CC]

In some embodiments, —X—R^{L} is

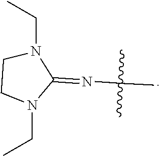 [SMILES: *N=C1N(CC)CCN1CC]

In some embodiments, —X—R^{L} is

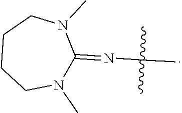 [SMILES: *N=C1N(C)CCCCN1C]

In some embodiments, —X—R^{L} is

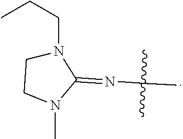 [SMILES: *N=C1N(C)CCN1CCC]

In some embodiments, —X—R^{b} is

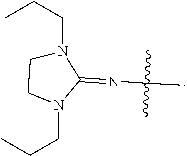 [SMILES: *N=C1N(CCC)CCN1CCC]

In some embodiments, —X—R^{L} is

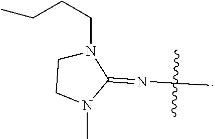 [SMILES: *N=C1N(C)CCN1CCCC]

In some embodiments, —X—R^{L} is

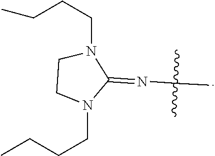 [SMILES: *N=C1N(CCCC)CCN1CCCC]

In some embodiments, —X—R^{L} is

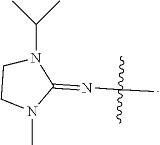 [SMILES: *N=C1N(C)CCN1C(C)C]

In some embodiments, —X—R^{L} is

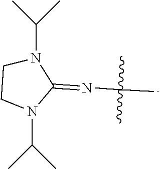 [SMILES: *N=C1N(C(C)C)CCN1C(C)C]

In some embodiments, —X—R^{L} is

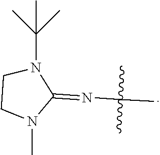 [SMILES: *N=C1N(C)CCN1C(C)(C)C]

In some embodiments, —X—R^{L} is

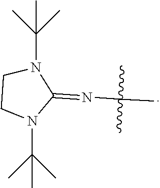 [SMILES: *N=C1N(C(C)(C)C)CCN1C(C)(C)C]

In some embodiments, —X—R^{L} is

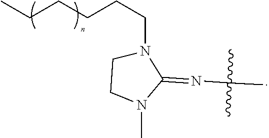

wherein n is 1-20. In some embodiments, —X—R^{L} is

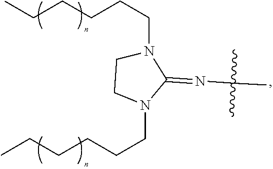

wherein n is 1-20.

In some embodiments, —X—R^{L} is R as described herein. In some embodiments, R is not hydrogen. In some embodiments, R is optionally substituted C₁₋₆ aliphatic. In some embodiments, R is optionally substituted C₁₋₆ alkyl. In some embodiments, R is methyl.

In some embodiments, an internucleotidic linkage, e.g., a non-negatively charged internucleotidic linkage or neutral internucleotidic linkage, has the structure of —OP(═W)(—N═C(R″)₂)—O—, —OP(═W)(—N(R″)₂)—O—, —P(═W)(—N═C(R″)₂)—O— or —P(═W)(—N(R″)₂)—O—, wherein:

W is O or S;

each R^{M1} is independently R′ or —N(R′)₂;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or:

two R groups are optionally and independently taken together to form a covalent bond, or:

two or more R groups on the same atom are optionally and independently taken together with the atom to form an optionally substituted, 3-30 membered monocyclic, bicyclic or polycyclic ring having, in addition to the atom, 0-10 heteroatoms, or:

two or more R groups on two or more atoms are optionally and independently taken together with their intervening atoms to form an optionally substituted, 3-30 membered monocyclic, bicyclic or polycyclic ring having, in addition to the intervening atoms, 0-10 heteroatoms.

In some embodiments, W is O. In some embodiments, W is S.

In some embodiments, Y is a covalent bond. In some embodiments, Y is —O—. In some embodiments, Y is —N(R′)—. In some embodiments, Z is a covalent bond. In some embodiments, Z is —O—. In some embodiments, Z is —N(R′)—. In some embodiments, R′ is R. In some embodiments, R is —H. In some embodiments, R is optionally substituted C₁₋₆ aliphatic. In some embodiments, R is methyl. In some embodiments, R is ethyl. In some embodiments, R is propyl. In some embodiments, R is optionally substituted phenyl. In some embodiments, R is phenyl.

As described herein, various variables in structures in the present disclosure can be or comprise R. Suitable embodiments for R are described extensively in the present disclosure. As appreciated by those skilled in the art, R embodiments described for a variable that can be R may also be applicable to another variable that can be R. Similarly, embodiments described for a component/moiety (e.g., L) for a variable may also be applicable to other variables that can be or comprise the component/moiety.

In some embodiments, R^{M1} is R′. In some embodiments, R^{M1} is —N(R′)₂.

In some embodiments, —X—R^{L} is —SH. In some embodiments, —X—R^{L} is —OH.

In some embodiments, —X—R^{L} is —N(R′)₂. In some embodiments, each R′ is independently optionally substituted C₁₋₆ aliphatic. In some embodiments, each R′ is independently methyl.

In some embodiments, a non-negatively charged internucleotidic linkage has the structure of —OP(═O)(—N═C((N(R′)₂)₂—O—. In some embodiments, a R′ group of one N(R′) 2 is R, a R′ group of the other N(R′) 2 is R, and the two R groups are taken together with their intervening atoms to form an optionally substituted ring, e.g., a 5-membered ring as in n001. In some embodiments, each R′ is independently R, wherein each R is independently optionally substituted C₁₋₆ aliphatic.

In some embodiments, —X—R^{L} is —N═C(-L^{L}-R′)₂. In some embodiments, —X—R^{L} is —N═C(-L^{L1}-L^{L2}-L^{L3}-R′)₂, wherein each L^{L1}, L^{L2} and L^{L3} is independently L″, wherein each L″ is independently a covalent bond, or a bivalent, optionally substituted, linear or branched group selected from a C₁₋₁₀ aliphatic group and a C₁₋₁₀ heteroaliphatic group having 1-5 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L^{L2} is -Cy-. In some embodiments, L^{L1} is a covalent bond. In some embodiments, L^{L3} is a covalent bond. In some embodiments, —X—R^{L} is —N═C(-L^{L1}-Cy-L^{L3}; —R′)₂. In some embodiments, —X—R^{L} is

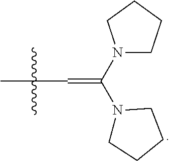 [SMILES: *C=C(N1CCCC1)N1CCCC1]

In some embodiments, —X—R^{L} is

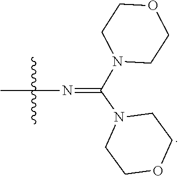 [SMILES: *N=C(N1CCOCC1)N1CCOCC1]

In some embodiments, —X—R^{L} is

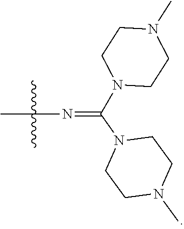 [SMILES: *N=C(N1CCN(C)CC1)N1CCN(C)CC1]

In some embodiments, —X—R^{L} is

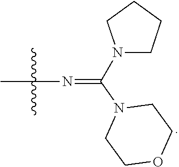 [SMILES: *N=C(N1CCCC1)N1CCOCC1]

In some embodiments, —X—R^{L} is

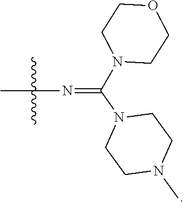 [SMILES: *N=C(N1CCOCC1)N1CCN(C)CC1]

In some embodiments, —X—R^{L} is

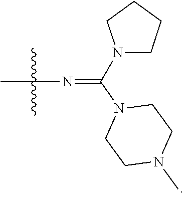 [SMILES: *N=C(N1CCCC1)N1CCN(C)CC1]

In some embodiments, as utilized in the present disclosure, L is covalent bond. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁-₆ alkenylene, ^{-CEC-}, a bivalent C₁-C₆ heteroaliphatic group having 1-5 heteroatoms, —C(R′)₂—, -Cy-, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)—, —OP(SR′)—, —OP(NR′)—, —OP(R′)—, or —OP(OR′)[B(R′)₃]—, and one or more nitrogen or carbon atoms are optionally and independently replaced with Cy^{L}. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by a group selected from C₁₋₆ alkylene, C₁₋₆ alkenylene, —C≡C—, a bivalent C₁-C6 heteroaliphatic group having 1-5 heteroatoms, —C(R′)₂—, -Cy-, —O—, —S—, —S—S-, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)₅—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)—, —OP(SR′)—, —OP(NR′)—, —OP(R′)—, or —OP(OR′)[B(R′)₃]O—. In some embodiments, L is a bivalent, optionally substituted, linear or branched group selected from a C₁₋₂₀ aliphatic group and a C₁₋₂₀ heteroaliphatic group having 1-10 heteroatoms, wherein one or more methylene units are optionally and independently replaced by a group selected from C₁₋₆ alkylene, C₁₋₆ alkenylene, —C≡C—, a bivalent C₁-C₆ heteroaliphatic group having 1-5 heteroatoms, —C(R′)₂—, -Cy-, ——, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)O—, —OP(SR′)—, —OP(NR′)—, —OP(R′)—, or —OP(OR′)[B(R′)₃]O—. In some embodiments, L is a bivalent, optionally substituted, linear or branched group selected from a C₁₋₁₀ (e.g., 1, 2, 3, 4, 5, 6, 7, 8, 9, or 10, 1-9, 1-8, 1-7, 1-6, etc.) aliphatic group and a C₁₋₁₀ (e.g., 1, 2, 3, 4, 5, 6, 7, 8, 9, or 10, 1-9, 1-8, 1-7, 1-6, etc.) heteroaliphatic group having 1-5 (e.g., 1, 2, 3, 4, or 5) heteroatoms, wherein one or more methylene units are optionally and independently replaced by a group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S-, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)O—, —OP(OR′)—, —OP(SR′)—, —OP(NR′)—, —OP(R′)—, or —OP(OR′)[B(R′)₃]O—. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)O—, —OP(O)(NR′)—, —OP(OR′)—, —OP(SR′)—, —OP(NR′)—, —OP(R′)—, or —OP(OR′)[B(R′)₃]O—, and one or more nitrogen or carbon atoms are optionally and independently replaced with Cy^{L}. In some embodiments, L is a bivalent, optionally substituted, linear or branched group selected from a C₁₋₁₀ aliphatic group and a C₁₋₁₀ heteroaliphatic group having 1-10 heteroatoms, wherein one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)—, —OP(O)(R′)—, —OP(O)(NR′)—, —OP(OR′)—, —OP(SR′)—, —OP(NR′)O—, —OP(R′)—, or —OP(OR′)[B(R′)₃]O—, and one or more nitrogen or carbon atoms are optionally and independently replaced with Cy^{L}. In some embodiments, one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L is a bivalent, optionally substituted, linear or branched group selected from a C₁₋₁₀ aliphatic group and a C₁₋₁₀ heteroaliphatic group having 1-5 heteroatoms, wherein one or more methylene units are optionally and independently replaced by —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)—, —OP(O)(R′)—, —OP(O)(NR′)—, —OP(OR′)—, —OP(SR′)—, —OP(NR′)O—, —OP(R′)—, or —OP(OR′)[B(R′)₃]O—. In some embodiments, L is a bivalent, optionally substituted, linear or branched group selected from a C₁₋₁₀ aliphatic group and a C₁₋₁₀ heteroaliphatic group having 1-5 heteroatoms, wherein one or more methylene units are optionally and independently replaced by —≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, or —C(O)O—.

In some embodiments, —X—R^{L} is —N═C[N(R′)₂]₂. In some embodiments, each R′ is independently R. In some embodiments, R is optionally substituted C₁₋₆ aliphatic. In some embodiments, R is methyl. In some embodiments, —X—R^{L} is

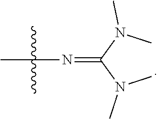 [SMILES: *N=C(N(C)C)N(C)C]

In some embodiments, one R′ on a nitrogen atom is taken with a R′ on the other nitrogen to form a ring as described herein. In some embodiments, a formed ring is optionally substituted 5-10 membered ring having 0-3 additional heteroatoms in addition to the two nitrogen atoms. In some embodiments, a formed ring is optionally substituted 5-10 membered ring having no additional heteroatoms in addition to the two nitrogen atoms. In some embodiments, a formed ring is optionally substituted 5-membered ring having no additional heteroatoms in addition to the two nitrogen atoms. In some embodiments, a formed ring is optionally substituted 6-membered ring having no additional heteroatoms in addition to the two nitrogen atoms. In some embodiments, a formed ring is optionally substituted 7-membered ring having no additional heteroatoms in addition to the two nitrogen atoms. In some embodiments, a formed ring is optionally substituted 8-membered ring having no additional heteroatoms in addition to the two nitrogen atoms. In some embodiments, a formed ring is monocyclic. In some embodiments, a formed ring is bicyclic. In some embodiments, a formed ring comprises no double or triple bond. In some embodiments, a formed ring comprises a double bond. In some embodiments, —X—R^{L} is optionally substituted

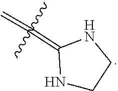 [SMILES: *=C1NCCN1]

In some embodiments, —X—R^{L} is

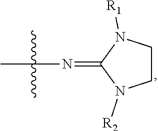

wherein R¹ and R² are independently R′. In some embodiments, —X—R^{L} is

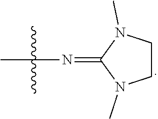 [SMILES: *N=C1N(C)CCN1C]

In some embodiments, —X—R^{L} is

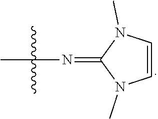 [SMILES: *N=c1n(C)ccn1C]

In some embodiments, two R′ on the same nitrogen are taken together to form a ring as described herein. In some embodiments, —X—R^{L} is

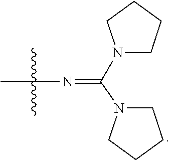 [SMILES: *N=C(N1CCCC1)N1CCCC1]

In some embodiments, —X—R^{L} is

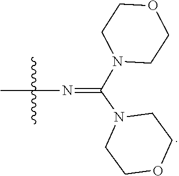 [SMILES: *N=C(N1CCOCC1)N1CCOCC1]

In some embodiments, —X—R^{L} is

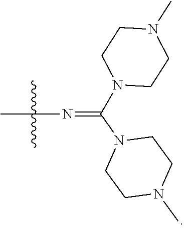 [SMILES: *N=C(N1CCN(C)CC1)N1CCN(C)CC1]

In some embodiments, —X—R^{L} is

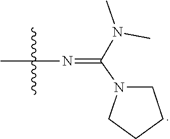 [SMILES: *N=C(N(C)C)N1CCCC1]

In some embodiments, —X—R^{L} is

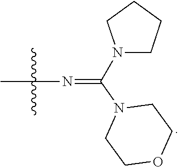 [SMILES: *N=C(N1CCCC1)N1CCOCC1]

In some embodiments, —X—R^{L} is

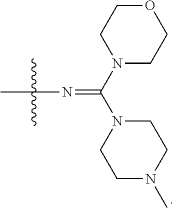 [SMILES: *N=C(N1CCOCC1)N1CCN(C)CC1]

In some embodiments, —X—R^{L} is

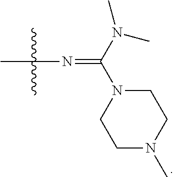 [SMILES: *N=C(N(C)C)N1CCN(C)CC1]

In some embodiments, —X—R^{L} is

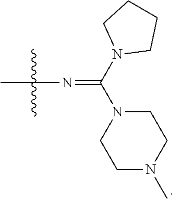 [SMILES: *N=C(N1CCCC1)N1CCN(C)CC1]

In some embodiments, —X—R^{L} is

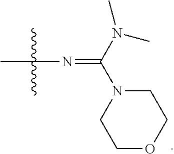 [SMILES: *N=C(N(C)C)N1CCOCC1]

In some embodiments, —X—R^{L} is

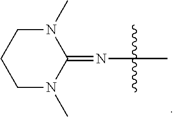 [SMILES: *N=C1N(C)CCCN1C]

In some embodiments, —X—R^{L} is

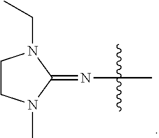 [SMILES: *N=C1N(C)CCN1CC]

In some embodiments, —X—R^{L} is

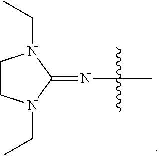 [SMILES: *N=C1N(CC)CCN1CC]

In some embodiments, —X—R^{L} is

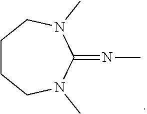

In some embodiments, —X—R^{L} is

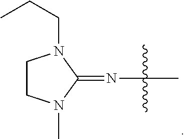 [SMILES: *N=C1N(C)CCN1CCC]

In some embodiments, —X—R^{L} is

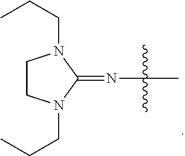 [SMILES: *N=C1N(CCC)CCN1CCC]

In some embodiments, —X—R^{L} is

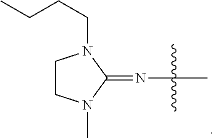 [SMILES: *N=C1N(C)CCN1CCCC]

In some embodiments, —X—R^{L} is

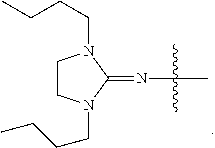 [SMILES: *N=C1N(CCCC)CCN1CCCC]

In some embodiments, —X—R^{L} is

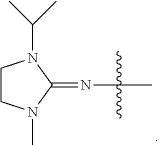 [SMILES: *N=C1N(C)CCN1C(C)C]

In some embodiments, —X—R^{L} is

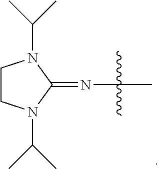 [SMILES: *N=C1N(C(C)C)CCN1C(C)C]

In some embodiments, —X—R^{L} is

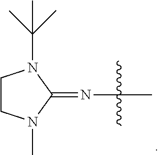 [SMILES: *N=C1N(C)CCN1C(C)(C)C]

In some embodiments, —X—R^{L} is

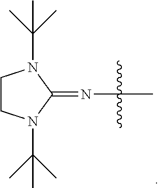 [SMILES: *N=C1N(C(C)(C)C)CCN1C(C)(C)C]

In some embodiments, —X—R^{L} is

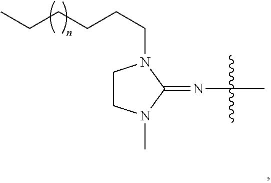

wherein n is 1-20. In some embodiments, —X—R^{L} is

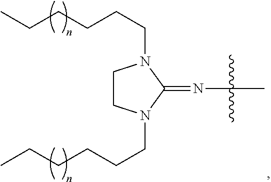

wherein n is 1-20. In some embodiments, —X—R^{L} is selected from

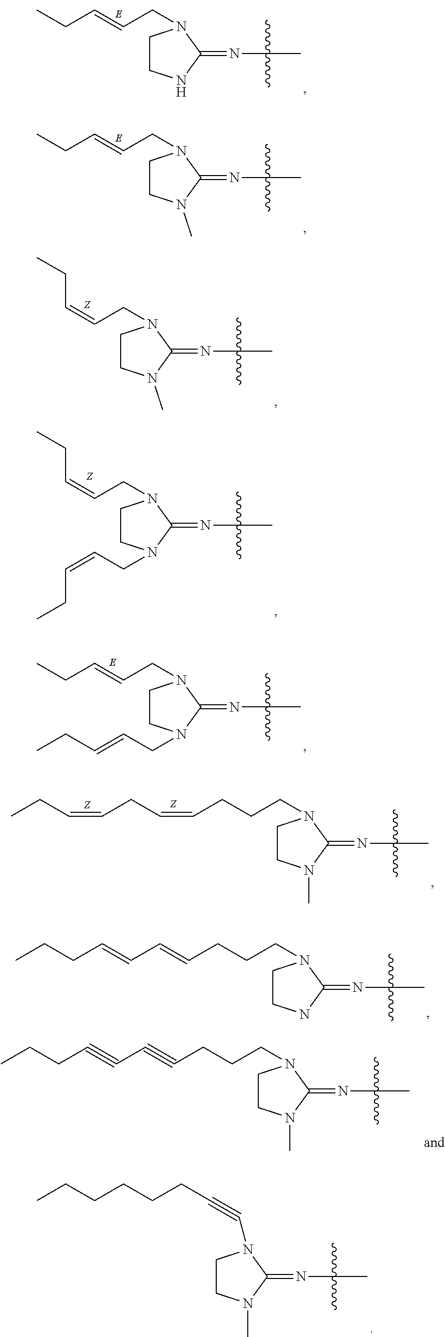

In some embodiments, —X—R^{L} is

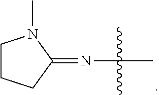 [SMILES: *N=C1CCCN1C]

In some embodiments, —X—R^{L} is

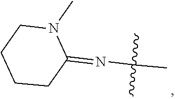 [SMILES: *N=C1CCCCN1C]

In some embodiments, —X—R^{L} is

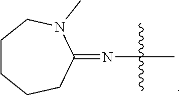 [SMILES: *N=C1CCCCCN1C]

In some embodiments, —X—R^{L} is optionally substituted

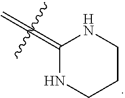 [SMILES: *=C1NCCCN1]

In some embodiments, —X—R^{L} is optionally substituted

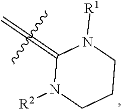

wherein each of R¹ and R² is independently R′ as described herein. In some embodiments, —X—R^{L} is optionally substituted

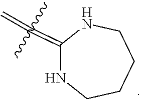 [SMILES: *=C1NCCCCN1]

In some embodiments, —X—R^{L} is optionally substituted

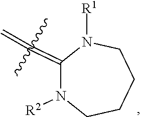

wherein each of R¹ and R² is independently R′ as described herein. In some embodiments, R¹ is R as described herein. In some embodiments, R¹ is optionally substituted C₁₋₃₀, C₁₋₂₀, C₁₋₁₀, or C₁₋₆ aliphatic. In some embodiments, R¹ is methyl. In some embodiments, R² is R as described herein. In some embodiments, R² is optionally substituted C₁₋₃₀, C₁₋₂₀, C₁₋₁₀, or C₁₋₆ aliphatic. In some embodiments, R² is methyl.

In some embodiments, —X—R^{L} is selected from Tables below. In some embodiments, X is as described herein. In some embodiments, R^{L} is as described herein. In some embodiments, a linkage has the structure of —Y—P^{L}(—X—R^{L})—Z—, wherein —X—R^{L} is selected from Tables below, and each other variable is independently as described herein. In some embodiments, a linkage has the structure of or comprises —P(O)(—X—R^{L})—, wherein —X—R^{L} is selected from Tables below. In some embodiments, a linkage has the structure of or comprises —P(S)(—X—R^{L})—, wherein —X—R^{L} is selected from Tables below. In some embodiments, a linkage has the structure of or comprises —P(—X—R^{L})—, wherein —X—R^{L} is selected from Tables below. In some embodiments, a linkage has the structure of or comprises —P(O)(—X—R^{L})—O—, wherein —X—R^{L} is selected from Tables below. In some embodiments, a linkage has the structure of or comprises —P(S)(—X—R^{L})—O—, wherein —X—R^{L} is selected from Tables below. In some embodiments, a linkage has the structure of or comprises —P(—X—R^{L})—O—, wherein —X—R^{L} is selected from Tables below. In some embodiments, a linkage has the structure of —P(O)(—X—R^{L})—O—, wherein —X—R^{L} is selected from Tables below. In some embodiments, a linkage has the structure of —P(S)(—X—R^{L})—O—, wherein —X—R^{L} is selected from Tables below. In some embodiments, a linkage has the structure of —P(—X—R^{L})—O—, wherein —X—R^{L} is selected from Tables below. In some embodiments, P is bonded to a nitrogen atom (e.g., a nitrogen atom in sm0 1). In some embodiments, a linkage has the structure of or comprises —O—P(O)(—X—R^{L})—O—, wherein —X—R^{L} is selected from Tables below. In some embodiments, a linkage has the structure of or comprises —O—P(S)(—X—R^{L})—O—, wherein —X—R^{L} is selected from Tables below. In some embodiments, a linkage has the structure of or comprises —O—P(—X—R^{L})—O—, wherein —X—R^{L} is selected from Tables below. In some embodiments, a linkage has the structure of —O—P(O)(—X—R^{L})—O—, wherein —X—R^{L} is selected from Tables below. In some embodiments, a linkage has the structure of —O—P(S)(—X—R^{L})—O—, wherein —X—R^{L} is selected from Tables below. In some embodiments, a linkage has the structure of —O—P(—X—R^{L})—O—, wherein —X—R^{L} is selected from Tables below. In some embodiments, the Tables below, n is 0-20 or as described herein. As those skilled in the art appreciate, a linkage may exist in a salt form.

TABLE L-1
Certain useful moieties bonded to linkage phosphorus (e.g., —X—R^{L}).

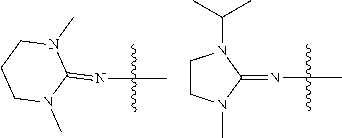

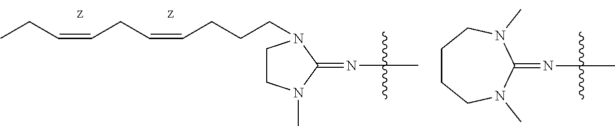

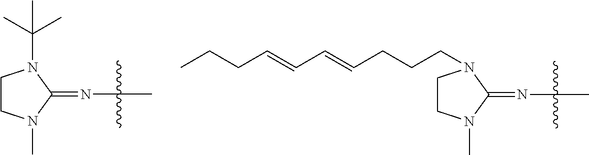

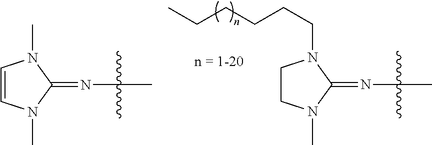

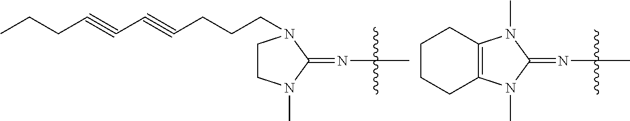

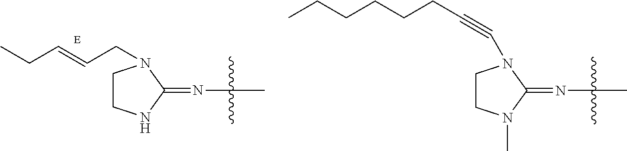

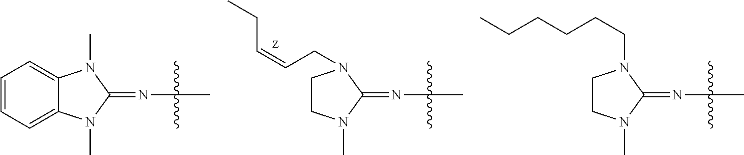

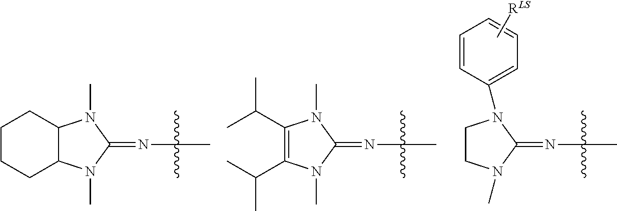

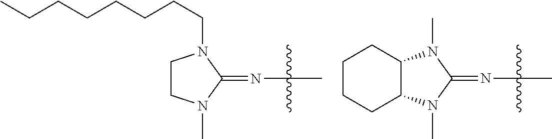

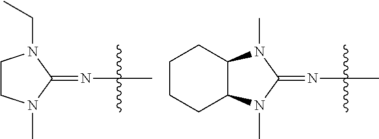

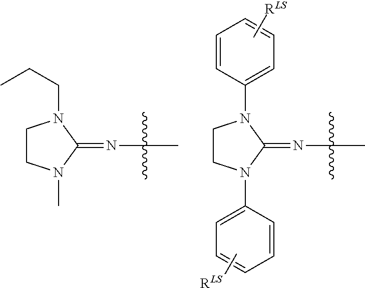

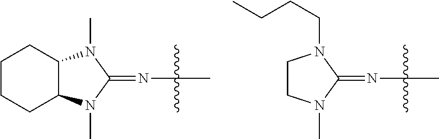

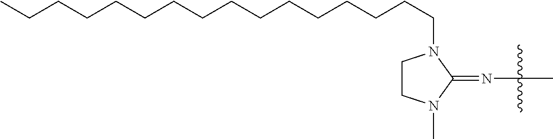

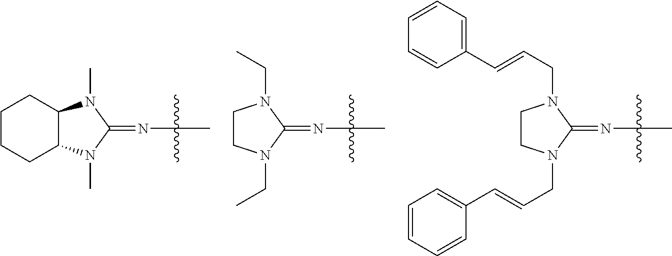

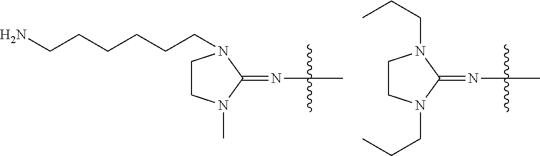

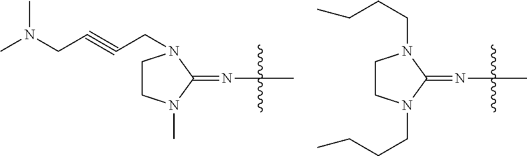

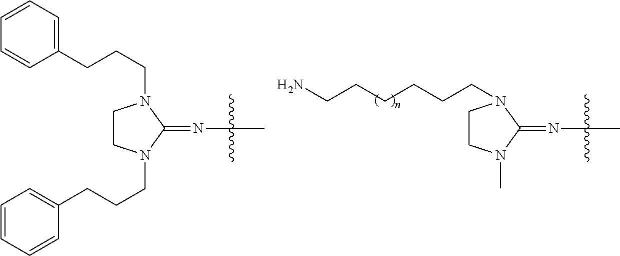

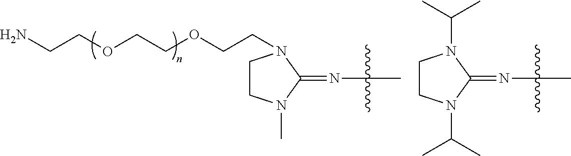

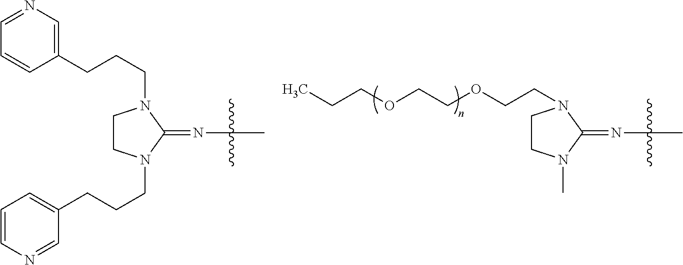

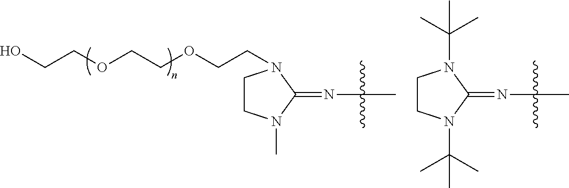

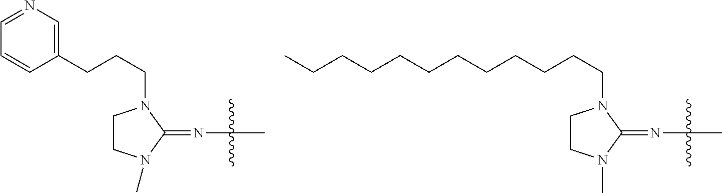

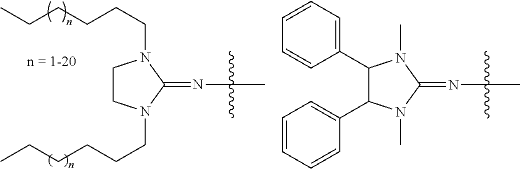

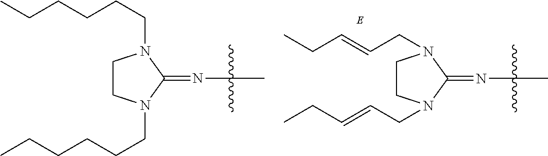

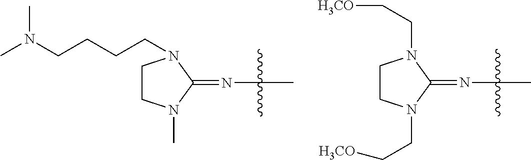

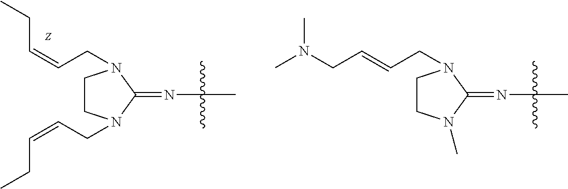

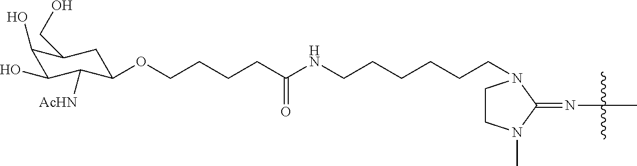

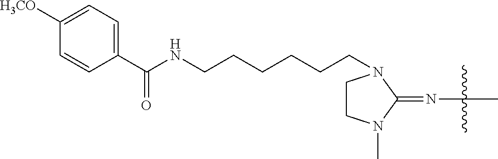

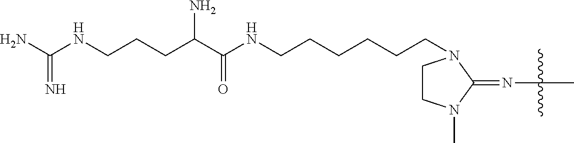

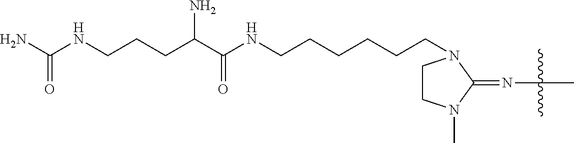

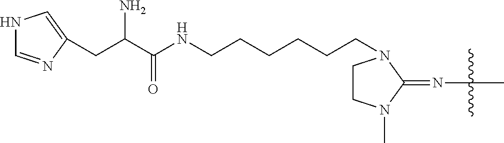

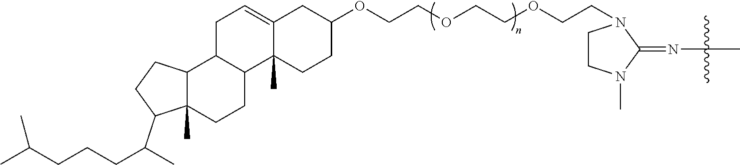

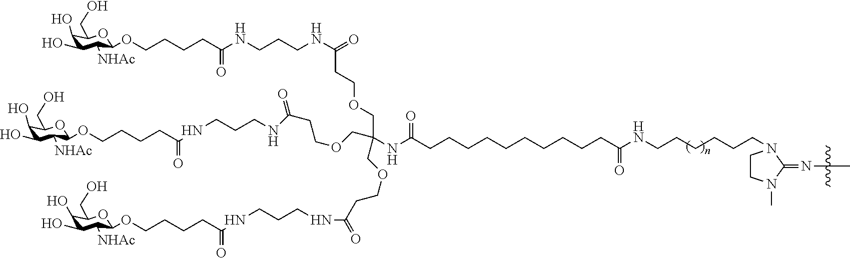

wherein each R^{LS} is independently R^{s}. In some embodiments, each R^{LS} is independently —Cl, —Br, —F, —N(Me)₂, or —NHCOCH₃.

TABLE L-2
Certain useful moieties bonded to linkage
phosphorus (e.g., —X—R^{L}).

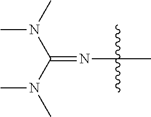 [SMILES: *N=C(N(C)C)N(C)C]

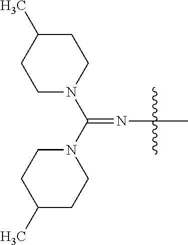 [SMILES: *N=C(N1CCC(C)CC1)N1CCC(C)CC1]

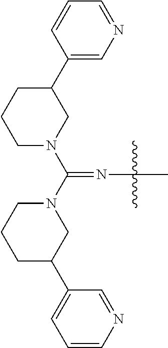 [SMILES: *N=C(N1CCCC(c2cccnc2)C1)N1CCCC(c2cccnc2)C1]

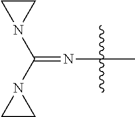 [SMILES: *N=C(N1CC1)N1CC1]

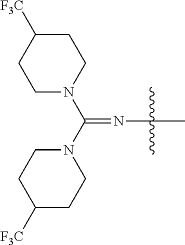 [SMILES: *N=C(N1CCC(C(F)(F)F)CC1)N1CCC(C(F)(F)F)CC1]

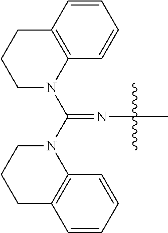 [SMILES: *N=C(N1CCCc2ccccc21)N1CCCc2ccccc21]

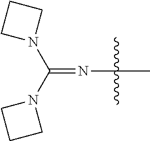 [SMILES: *N=C(N1CCC1)N1CCC1]

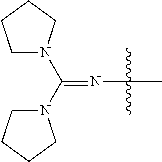 [SMILES: *N=C(N1CCCC1)N1CCCC1]

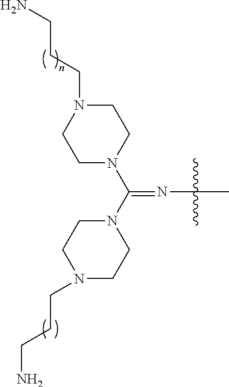

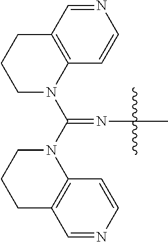 [SMILES: *N=C(N1CCCc2cnccc21)N1CCCc2cnccc21]

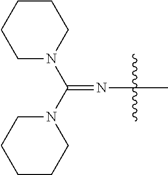 [SMILES: *N=C(N1CCCCC1)N1CCCCC1]

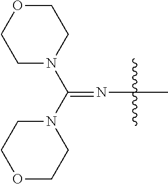 [SMILES: *N=C(N1CCOCC1)N1CCOCC1]

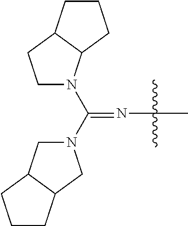 [SMILES: *N=C(N1CC2CCCC2C1)N1CCC2CCCC21]

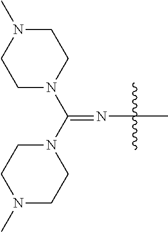 [SMILES: *N=C(N1CCN(C)CC1)N1CCN(C)CC1]

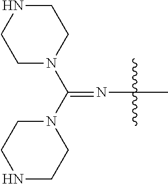 [SMILES: *N=C(N1CCNCC1)N1CCNCC1]

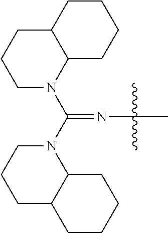 [SMILES: *N=C(N1CCCC2CCCCC21)N1CCCC2CCCCC21]

TABLE L-3
Certain useful moieties bonded
to linkage phosphorus
(e.g., —X—R^{L}).

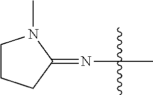 [SMILES: *N=C1CCCN1C]

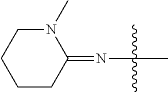 [SMILES: *N=C1CCCCN1C]

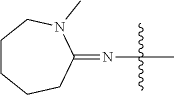 [SMILES: *N=C1CCCCCN1C]

TABLE L-4
Certain useful moieties bonded to linkage phosphorus
(e.g., —X—R^{L}).

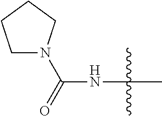 [SMILES: *NC(=O)N1CCCC1]

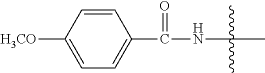 [SMILES: *NC(=O)c1ccc(OC)cc1]

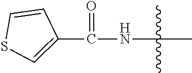 [SMILES: *NC(=O)c1ccsc1]

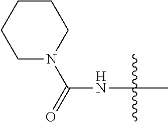 [SMILES: *NC(=O)N1CCCCC1]

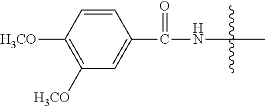 [SMILES: *NC(=O)c1ccc(OC)c(OC)c1]

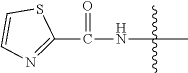 [SMILES: *NC(=O)c1nccs1]

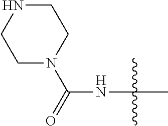 [SMILES: *NC(=O)N1CCNCC1]

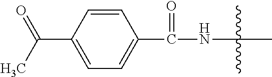 [SMILES: *NC(=O)c1ccc(C(C)=O)cc1]

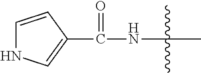 [SMILES: *NC(=O)c1cc[nH]c1]

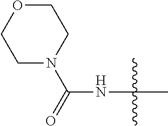 [SMILES: *NC(=O)N1CCOCC1]

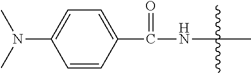 [SMILES: *NC(=O)c1ccc(N(C)C)cc1]

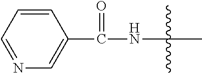 [SMILES: *NC(=O)c1cccnc1]

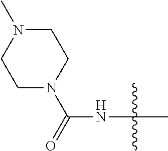 [SMILES: *NC(=O)N1CCN(C)CC1]

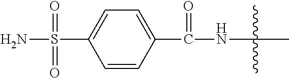 [SMILES: *NC(=O)c1ccc(S(N)(=O)=O)cc1]

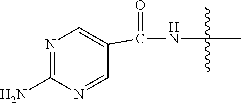 [SMILES: *NC(=O)c1cnc(N)nc1]

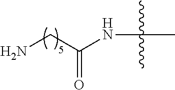

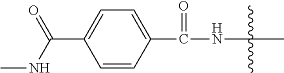 [SMILES: *NC(=O)c1ccc(C(=O)NC)cc1]

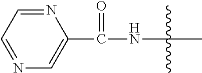 [SMILES: *NC(=O)c1cnccn1]

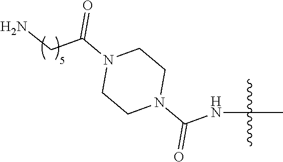

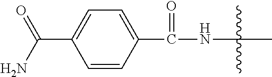 [SMILES: *NC(=O)c1ccc(C(N)=O)cc1]

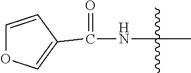 [SMILES: *NC(=O)c1ccoc1]

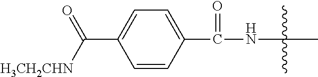

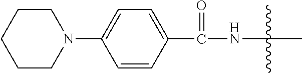 [SMILES: *NC(=O)c1ccc(N2CCCCC2)cc1]

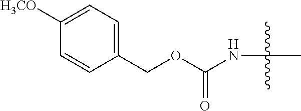 [SMILES: *NC(=O)OCc1ccc(OC)cc1]

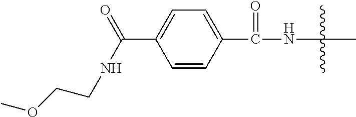 [SMILES: *NC(=O)c1ccc(C(=O)NCCOC)cc1]

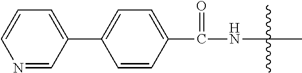 [SMILES: *NC(=O)c1ccc(-c2cccnc2)cc1]

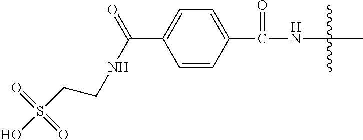 [SMILES: *NC(=O)c1ccc(C(=O)NCCS(=O)(=O)O)cc1]

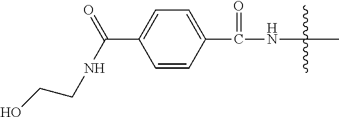 [SMILES: *NC(=O)c1ccc(C(=O)NCCO)cc1]

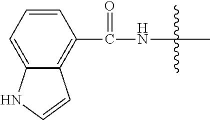 [SMILES: *NC(=O)c1cccc2[nH]ccc12]

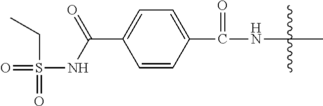 [SMILES: *NC(=O)c1ccc(C(=O)NS(=O)(=O)CC)cc1]

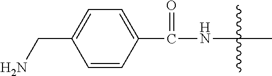 [SMILES: *NC(=O)c1ccc(CN)cc1]

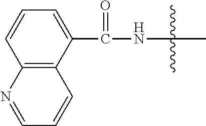 [SMILES: *NC(=O)c1cccc2ncccc12]

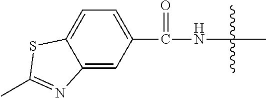 [SMILES: *NC(=O)c1ccc2sc(C)nc2c1]

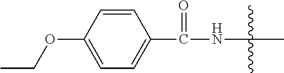 [SMILES: *NC(=O)c1ccc(OCC)cc1]

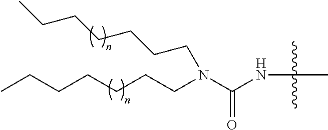

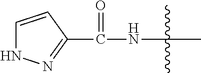 [SMILES: *NC(=O)c1cc[nH]n1]

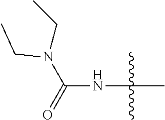 [SMILES: *NC(=O)N(CC)CC]

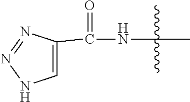 [SMILES: *NC(=O)c1c[nH]nn1]

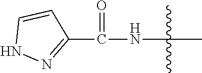 [SMILES: *NC(=O)c1cc[nH]n1]

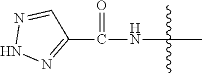 [SMILES: *NC(=O)c1cn[nH]n1]

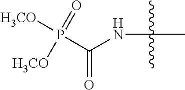 [SMILES: *NC(=O)P(=O)(OC)OC]

TABLE L-5
Certain useful moieties bonded to linkage phosphorus
(e.g., —X—R^{L}).

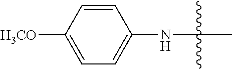 [SMILES: *Nc1ccc(OC)cc1]

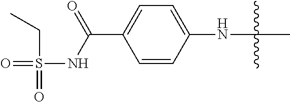 [SMILES: *Nc1ccc(C(=O)NS(=O)(=O)CC)cc1]

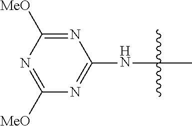 [SMILES: *Nc1nc(OC)nc(OC)n1]

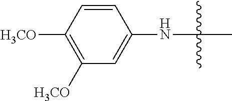 [SMILES: *Nc1ccc(OC)c(OC)c1]

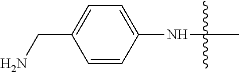 [SMILES: *Nc1ccc(CN)cc1]

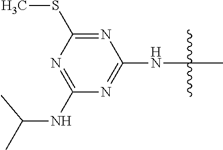 [SMILES: *Nc1nc(NC(C)C)nc(SC)n1]

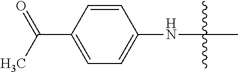 [SMILES: *Nc1ccc(C(C)=O)cc1]

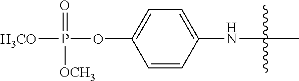 [SMILES: *Nc1ccc(OP(=O)(OC)OC)cc1]

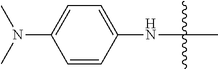 [SMILES: *Nc1ccc(N(C)C)cc1]

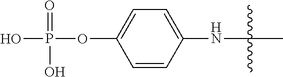 [SMILES: *Nc1ccc(OP(=O)(O)O)cc1]

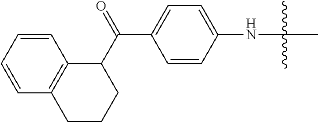 [SMILES: *Nc1ccc(C(=O)C2CCCc3ccccc32)cc1]

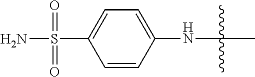 [SMILES: *Nc1ccc(S(N)(=O)=O)cc1]

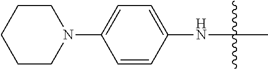 [SMILES: *Nc1ccc(N2CCCCC2)cc1]

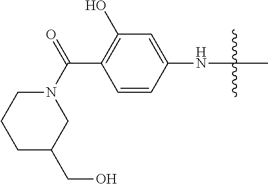 [SMILES: *Nc1ccc(C(=O)N2CCCC(CO)C2)c(O)c1]

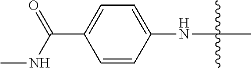 [SMILES: *Nc1ccc(C(=O)NC)cc1]

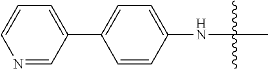 [SMILES: *Nc1ccc(-c2cccnc2)cc1]

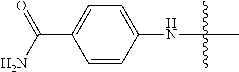 [SMILES: *Nc1ccc(C(N)=O)cc1]

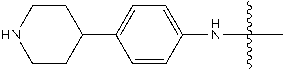 [SMILES: *Nc1ccc(C2CCNCC2)cc1]

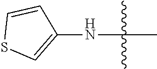 [SMILES: *Nc1ccsc1]

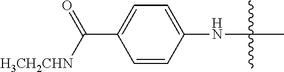

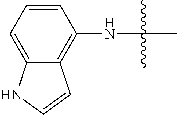 [SMILES: *Nc1cccc2[nH]ccc12]

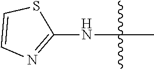 [SMILES: *Nc1nccs1]

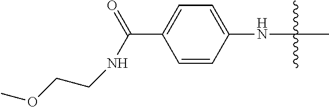 [SMILES: *Nc1ccc(C(=O)NCCOC)cc1]

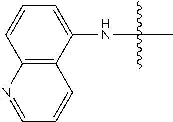 [SMILES: *Nc1cccc2ncccc12]

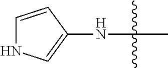 [SMILES: *Nc1cc[nH]c1]

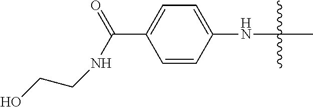 [SMILES: *Nc1ccc(C(=O)NCCO)cc1]

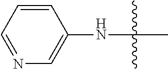 [SMILES: *Nc1cccnc1]

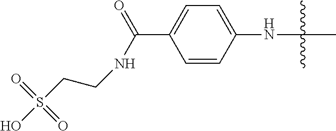 [SMILES: *Nc1ccc(C(=O)NCCS(=O)(=O)O)cc1]

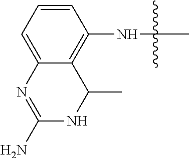 [SMILES: *Nc1cccc2c1C(C)NC(N)=N2]

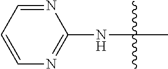 [SMILES: *Nc1ncccn1]

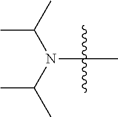 [SMILES: *N(C(C)C)C(C)C]

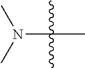 [SMILES: *N(C)C]

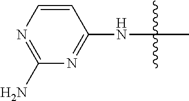 [SMILES: *Nc1ccnc(N)n1]

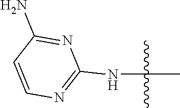 [SMILES: *Nc1nccc(N)n1]

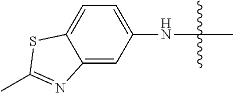 [SMILES: *Nc1ccc2sc(C)nc2c1]

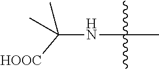 [SMILES: *NC(C)(C)C(=O)O]

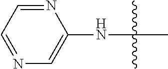 [SMILES: *Nc1cnccn1]

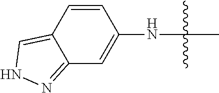 [SMILES: *Nc1ccc2c[nH]nc2c1]

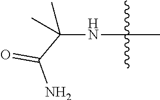 [SMILES: *NC(C)(C)C(N)=O]

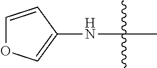 [SMILES: *Nc1ccoc1]

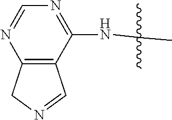 [SMILES: *Nc1ncnc2c1C=NC2]

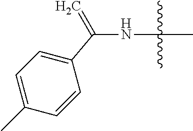 [SMILES: *NC(=C)c1ccc(C)cc1]

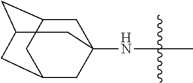 [SMILES: *NC12CC3CC(CC(C3)C1)C2]

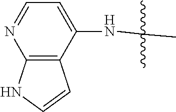 [SMILES: *Nc1ccnc2[nH]ccc12]

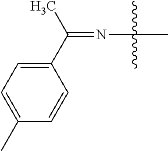 [SMILES: *N=C(C)c1ccc(C)cc1]

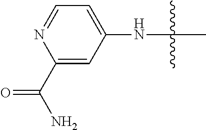 [SMILES: *Nc1ccnc(C(N)=O)c1]

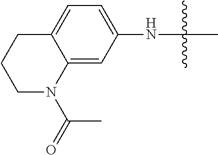 [SMILES: *Nc1ccc2c(c1)N(C(C)=O)CCC2]

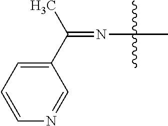 [SMILES: *N=C(C)c1cccnc1]

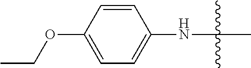 [SMILES: *Nc1ccc(OCC)cc1]

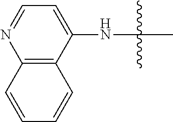 [SMILES: *Nc1ccnc2ccccc12]

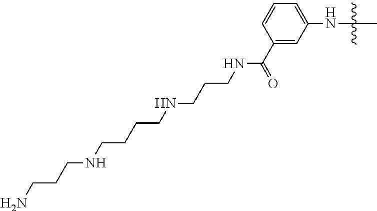 [SMILES: *Nc1cccc(C(=O)NCCCNCCCCNCCCN)c1]

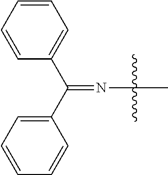 [SMILES: *N=C(c1ccccc1)c1ccccc1]

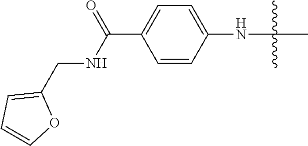 [SMILES: *Nc1ccc(C(=O)NCc2ccco2)cc1]

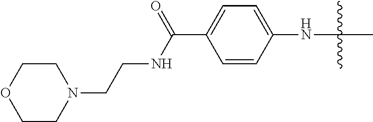 [SMILES: *Nc1ccc(C(=O)NCCN2CCOCC2)cc1]

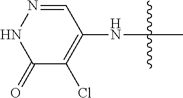 [SMILES: *Nc1cn[nH]c(=O)c1Cl]

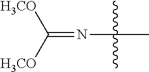 [SMILES: *N=C(OC)OC]

TABLE L-6
Certain useful moieties bonded to linkage
phosphorus (e.g., —X—R^{L} ).

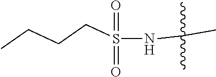 [SMILES: *NS(=O)(=O)CCCC]

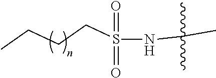

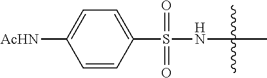 [SMILES: *NS(=O)(=O)c1ccc(NC(C)=O)cc1]

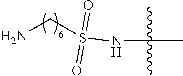

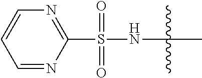 [SMILES: *NS(=O)(=O)c1ncccn1]

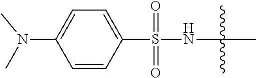 [SMILES: *NS(=O)(=O)c1ccc(N(C)C)cc1]

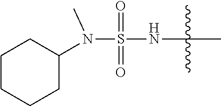 [SMILES: *NS(=O)(=O)N(C)C1CCCCC1]

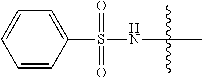 [SMILES: *NS(=O)(=O)c1ccccc1]

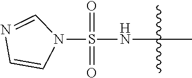 [SMILES: *NS(=O)(=O)n1ccnc1]

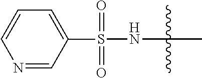 [SMILES: *NS(=O)(=O)c1cccnc1]

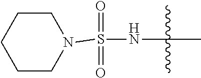 [SMILES: *NS(=O)(=O)N1CCCCC1]

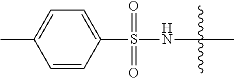 [SMILES: *NS(=O)(=O)c1ccc(C)cc1]

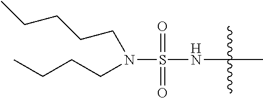 [SMILES: *NS(=O)(=O)N(CCCC)CCCCC]

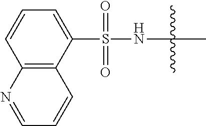 [SMILES: *NS(=O)(=O)c1cccc2ncccc12]

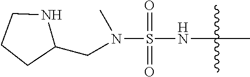 [SMILES: *NS(=O)(=O)N(C)CC1CCCN1]

In some embodiments, —X—R^{L} is —NHSO₂R′, wherein R′ is as described herein. In some embodiments, —X—R^{L} is —NHCOR′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl.

In some embodiments, an internucleotidic linkage, e.g., a non-negatively charged internucleotidic linkage or a neutral internucleotidic linkage, has the structure of -L^{L1}-Cy^{IL}-L^{L2}-. In some embodiments, L^{L1} is bonded to a 3′-carbon of a sugar. In some embodiments, L^{L2} is bonded to a 5′-carbon of a sugar. In some embodiments, L^{L1} is —O—CH₂—. In some embodiments, L^{L2} is a covalent bond. In some embodiments, L^{L2} is a —N(R′)—. In some embodiments, L^{L2} is a —NH—. In some embodiments, L^{L2} is bonded to a 5′-carbon of a sugar, which 5′-carbon is substituted with ═O. In some embodiments, Cy^{lL} is optionally substituted 3-10 membered saturated, partially unsaturated, or aromatic ring having 0-5 heteroatoms. In some embodiments, Cy^{IL} is an optionally substituted triazole ring. In some embodiments, Cy^{IL} is

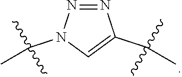 [SMILES: *c1cn(*)nn1]

In some embodiments, a linkage is

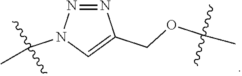 [SMILES: *OCc1cn(*)nn1]

In some embodiments, a non-negatively charged internucleotidic linkage has the structure of —OP(═W)(—N(R′)₂)—O—.

In some embodiments, R′ is R. In some embodiments, R′ is H. In some embodiments, R′ is —C(O)R. In some embodiments, R′ is —C(O)OR. In some embodiments, R′ is —S(O)₂R.

In some embodiments, R^{M1} is —NHR′. In some embodiments, —N(R′)₂ is —NHR′.

As described herein, some embodiments, R is H. In some embodiments, R is optionally substituted C₁₋₆ aliphatic. In some embodiments, R is optionally substituted C₁₋₆ alkyl. In some embodiments, R is methyl. In some embodiments, R is substituted methyl. In some embodiments, R is ethyl. In some embodiments, R is substituted ethyl.

In some embodiments, as described herein, a non-negatively charged internucleotidic linkage is a neutral internucleotidic linkage.

In some embodiments, a modified internucleotidic linkage (e.g., a non-negatively charged internucleotidic linkage) comprises optionally substituted triazolyl. In some embodiments, a modified internucleotidic linkage (e.g., a non-negatively charged internucleotidic linkage) comprises optionally substituted alkynyl. In some embodiments, a modified internucleotidic linkage comprises a triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted cyclic guanidine moiety. In some embodiments, a modified internucleotidic linkage comprises an optionally substituted cyclic guanidine moiety and has the structure of:

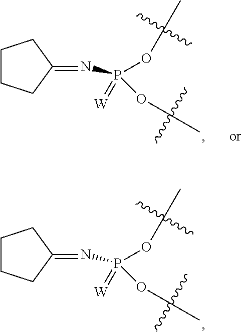

wherein W is O or S. In some embodiments, W is O. In some embodiments, W is S. In some embodiments, a non-negatively charged internucleotidic linkage is stereochemically controlled.

In some embodiments, a non-negatively charged internucleotidic linkage or a neutral internucleotidic linkage is an internucleotidic linkage comprising a triazole moiety. In some embodiments, a non-negatively charged internucleotidic linkage or a non-negatively charged internucleotidic linkage comprises an optionally substituted triazolyl group. In some embodiments, an internucleotidic linkage comprising a triazole moiety (e.g., an optionally substituted triazolyl group) has the structure of

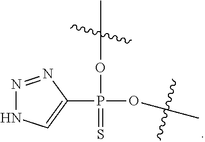 [SMILES: *OP(=S)(O*)c1c[nH]nn1]

In some embodiments, an internucleotidic linkage comprising a triazole moiety has the structure of

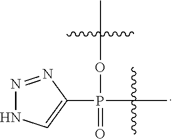 [SMILES: *OP(*)(=O)c1c[nH]nn1]

In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

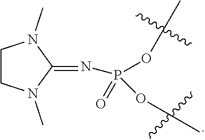 [SMILES: *OP(=O)(N=C1N(C)CCN1C)O*]

In some embodiments, a non-negatively charged internucleotidic linkage, or a neutral internucleotidic linkage, is or comprising a structure selected from

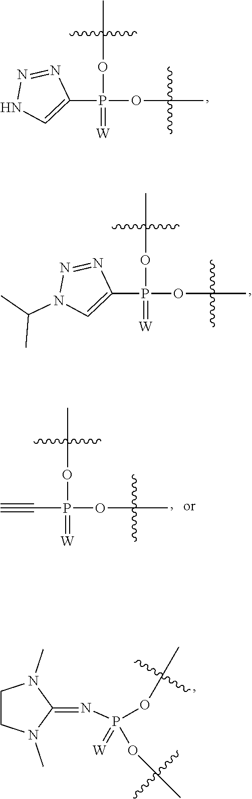

wherein W is O or S.

In some embodiments, an internucleotidic linkage comprises a Tmg group

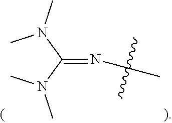 [SMILES: *N=C(N(C)C)N(C)C]

In some embodiments, an internucleotidic linkage comprises a Tmg group and has the structure of

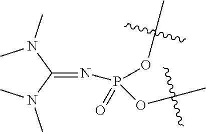 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, a non-negatively charged internucleotidic linkage has the structure of

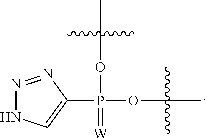

In some embodiments, a non-negatively charged internucleotidic linkage has the structure of

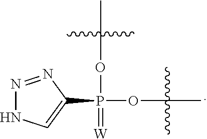

In some embodiments, a non-negatively charged internucleotidic linkage has the structure of

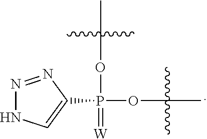

In some embodiments, a non-negatively charged internucleotidic linkage has the structure of

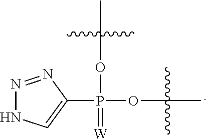

In some embodiments, a non-negatively charged internucleotidic linkage has the structure of

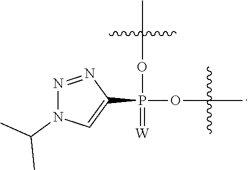

In some embodiments, a non-negatively charged internucleotidic linkage has the structure of

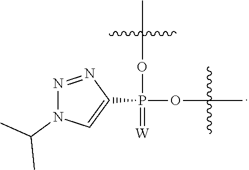

In some embodiments, a non-negatively charged internucleotidic linkage has the structure of

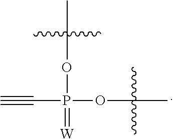

In some embodiments, a non-negatively charged internucleotidic linkage has the structure of

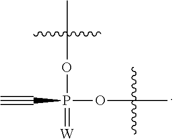

In some embodiments, a non-negatively charged internucleotidic linkage has the structure of

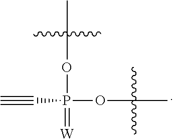

In some embodiments, W is O. In some embodiments, W is S. In some embodiments, a neutral internucleotidic linkage is a non-negatively charged internucleotidic linkage described above.

In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-10 heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a non-negatively charged internucleotidic linkage comprises an optionally substituted triazolyl group. In some embodiments, a non-negatively charged internucleotidic linkage comprises an unsubstituted triazolyl group, e.g.,

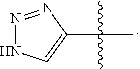 [SMILES: *c1c[nH]nn1]

In some embodiments, a non-negatively charged internucleotidic linkage comprises a substituted triazolyl group, e.g.,

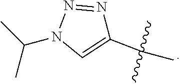 [SMILES: *c1cn(C(C)C)nn1]

In some embodiments, a heterocyclyl group is directly bonded to a linkage phosphorus. In some embodiments, a heterocyclyl group is bonded to a linkage phosphorus through a linker, e.g., ═N— when the heterocyclyl group is part of a guanidine moiety who directed bonded to a linkage phosphorus through its ═N—. In some embodiments, a non-negatively charged internucleotidic linkage comprises an optionally substituted

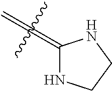 [SMILES: *=C1NCCN1]

group. In some embodiments, a non-negatively charged internucleotidic linkage comprises an substituted

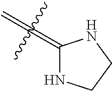 [SMILES: *=C1NCCN1]

group. In some embodiments, a non-negatively charged internucleotidic linkage comprises a

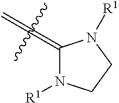

group. In some embodiments, a non-negatively charged internucleotidic linkage comprises an optionally substituted

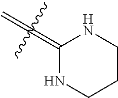 [SMILES: *=C1NCCCN1]

group. In some embodiments, a non-negatively charged internucleotidic linkage comprises an substituted

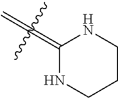 [SMILES: *=C1NCCCN1]

group. In some embodiments, a non-negatively charged internucleotidic linkage comprises a

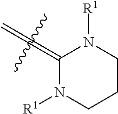

group. In some embodiments, a non-negatively charged internucleotidic linkage comprises an optionally substituted

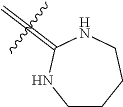 [SMILES: *=C1NCCCCN1]

group. In some embodiments, a non-negatively charged internucleotidic linkage comprises an substituted

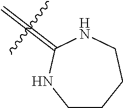 [SMILES: *=C1NCCCCN1]

group. In some embodiments, a non-negatively charged internucleotidic linkage comprises a

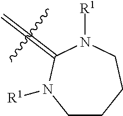

group. In some embodiments, each R′ is independently optionally substituted C₁₋₆ alkyl. In some embodiments, each R¹ is independently methyl. In some embodiments, —X—R^{L} is such a group.

In some embodiments, a non-negatively charged internucleotidic linkage, e.g., a neutral internucleotidic linkage is not chirally controlled. In some embodiments, a non-negatively charged internucleotidic linkage is chirally controlled. In some embodiments, a non-negatively charged internucleotidic linkage is chirally controlled and its linkage phosphorus is Rp. In some embodiments, a non-negatively charged internucleotidic linkage is chirally controlled and its linkage phosphorus is Sp.

In some embodiments, an internucleotidic linkage comprises no linkage phosphorus. In some embodiments, an internucleotidic linkage has the structure of —C(O)—(O)— or —C(O)—N(R′)—, wherein R′ is as described herein. In some embodiments, an internucleotidic linkage has the structure of —C(O)—(O)—. In some embodiments, an internucleotidic linkage has the structure of —C(O)—N(R′)—, wherein R′ is as described herein. In various embodiments, —C(O)— is bonded to nitrogen. In some embodiments, an internucleotidic linkage is or comprises —C(O)—O— which is part of a carbamate moiety. In some embodiments, an internucleotidic linkage is or comprises —C(O)—O— which is part of a urea moiety.

In some embodiments, an oligonucleotide comprises 1-20, 1-15, 1-10, 1-5, or 1, 2, 3, 4, 5, 6, 7, 8, 9, 10, or more non-negatively charged internucleotidic linkages. In some embodiments, an oligonucleotide comprises 1-20, 1-15, 1-10, 1-5, or 1, 2, 3, 4, 5, 6, 7, 8, 9, 10, or more neutral internucleotidic linkages. In some embodiments, each of non-negatively charged internucleotidic linkage and/or neutral internucleotidic linkages is optionally and independently chirally controlled. In some embodiments, each non-negatively charged internucleotidic linkage in an oligonucleotide is independently a chirally controlled internucleotidic linkage. In some embodiments, each neutral internucleotidic linkage in an oligonucleotide is independently a chirally controlled internucleotidic linkage. In some embodiments, at least one non-negatively charged internucleotidic linkage/neutral internucleotidic linkage has the structure of

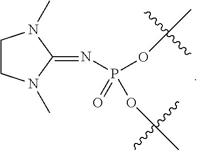 [SMILES: *OP(=O)(N=C1N(C)CCN1C)O*]

In some embodiments, an oligonucleotide comprises at least one non-negatively charged internucleotidic linkage wherein its linkage phosphorus is in Rp configuration, and at least one non-negatively charged internucleotidic linkage wherein its linkage phosphorus is in Sp configuration.

In many embodiments, as demonstrated extensively, oligonucleotides of the present disclosure comprise two or more different internucleotidic linkages. In some embodiments, an oligonucleotide comprises a phosphorothioate internucleotidic linkage and a non-negatively charged internucleotidic linkage. In some embodiments, an oligonucleotide comprises a phosphorothioate internucleotidic linkage, a non-negatively charged internucleotidic linkage, and a natural phosphate linkage. In some embodiments, a non-negatively charged internucleotidic linkage is a neutral internucleotidic linkage. In some embodiments, a non-negatively charged internucleotidic linkage is

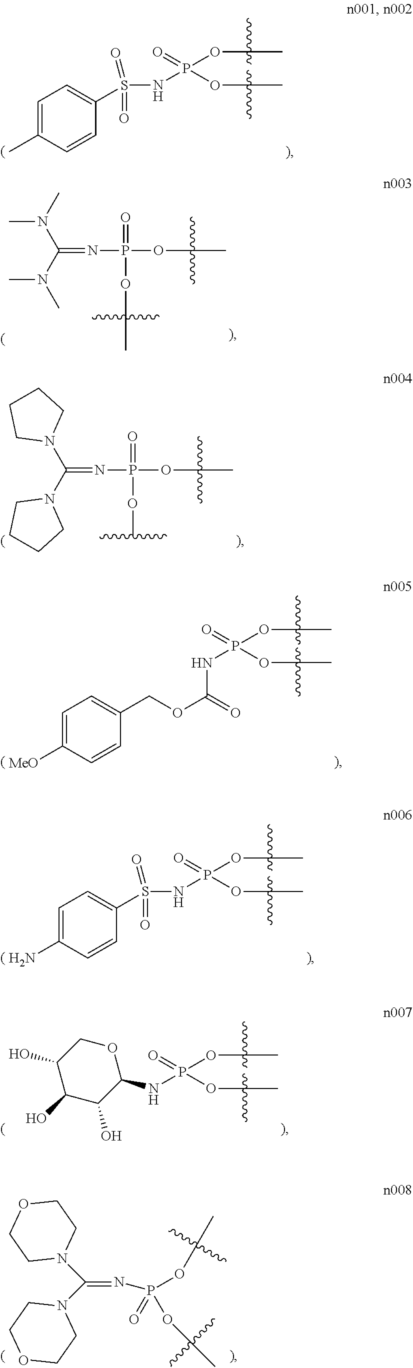

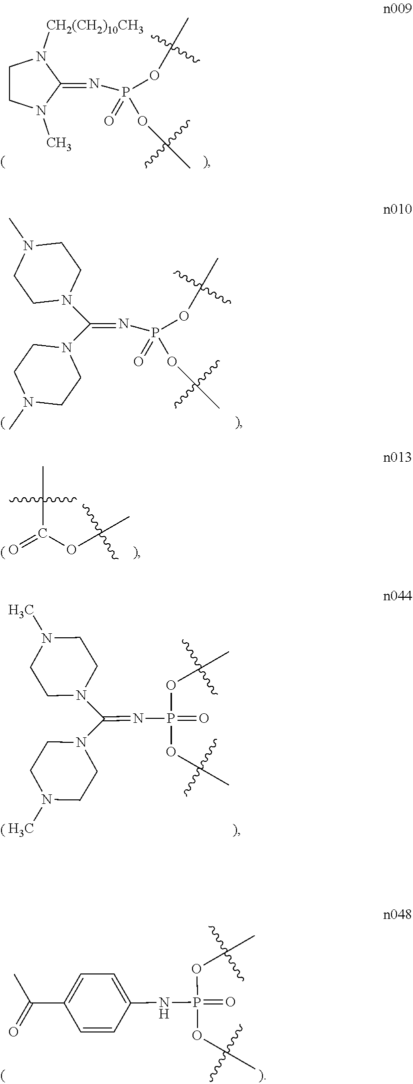

In some embodiments, a non-negatively charged internucleotidic linkage is n001. In some embodiments, each phosphorothioate internucleotidic linkage is independently chirally controlled. In some embodiments, each chiral modified internucleotidic linkage is independently chirally controlled. In some embodiments, one or more non-negatively charged internucleotidic linkage are not chirally controlled.

A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms with one optionally modified ribose or deoxyribose at its 5′ carbon, and the other optionally modified ribose or deoxyribose at its 3′ carbon. In some embodiments, internucleotidic linkages connect sugars that are not ribose sugars, e.g., sugars comprising N ring atoms and acyclic sugars as described herein.

In some embodiments, each nucleoside units connected by an internucleotidic linkage independently comprises a nucleobase which is independently an optionally substituted A, T, C, G, or U, or an optionally substituted tautomer of A, T, C, G or U.

In some embodiments, an oligonucleotide comprises a modified internucleotidic linkage (e.g., a modified internucleotidic linkage having the structure of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc., or a salt form thereof) as described in U.S. Pat. Nos. 9,394333, 9,744,183, 9,605,019, 9,598,458, 9,982,257, 10,160,969, 10,479,995, US 2020/0056173, 2018/0216107, 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858 the internucleotidic linkages (e.g., those of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc.,) of each of which are independently incorporated herein by reference. In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage or a neutral internucleotidic linkage (e.g., one of Formula I-n-1, I-n-2, I-n-3, I-n-4, II, II-a-1, II-a-2, II-b-1, II-b-2, II-c-1, II-c-2, II-d-1, II-d-2, etc.) is as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858. In some embodiments, a non-negatively charged internucleotidic linkage or neutral internucleotidic linkage is one of Formula I-n-1, I-n-2, I-n-3, I-n-4, II, II-a-1, II-a-2, II-b-1, II-b-2, II-c-1, II-c-2, II-d-1, II-d-2, etc. as described in WO 2018/223056, WO 2019/032607, WO 2019/075357, WO 2019/032607, WO 2019/075357, WO 2019/200185, WO 2019/217784, WO 2019/032612, WO 2020/191252, and/or WO 2021/071858, such internucleotidic linkages of each of which are independently incorporated herein by reference.

As appreciated by those skilled in the art, many other types of internucleotidic linkages may be utilized in accordance with the present disclosure, for example, those described in U.S. Pat. Nos. 3,687,808; 4,469,863; 4,476,301; 5,177,195; 5,023,243; 5,034,506; 5,166,315; 5,185,444; 5,188,897; 5,214,134; 5,216,141; 5,235,033; 5,264,423; 5,264,564; 5,276,019; 5,278,302; 5,286,717; 5,321,131; 5,399,676; 5,405,938; 5,405,939; 5,434,257; 5,453,496; 5,455,233; 5,466,677; 5,466,677; 5,470,967; 5,476,925; 5,489,677; 5,519,126; 5,536,821; 5,541,307; 5,541,316; 5,550,111; 5,561,225; 5,563,253; 5,571,799; 5,587,361; 5,596,086; 5,602,240; 5,608,046; 5,610,289; 5,618,704; 5,623,070; 5,625,050; 5,633,360; 5,64,562; 5,663,312; 5,677,437; 5,677,439; 6,160,109; 6,239,265; 6,028,188; 6,124,445; 6,169,170; 6,172,209; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; or RE39464. In some embodiments, a modified internucleotidic linkage is one described in U.S. Pat. Nos. 9,394333, 9,744,183, 9,605,019, 9,598,458, 9,982,257, 10,160,969, 10,479,995, US 2020/0056173, US 2018/0216107, US 2019/0127733, U.S. Pat. No. 10,450568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the nucleobases, sugars, internucleotidic linkages, chiral auxiliaries/reagents, and technologies for oligonucleotide synthesis (reagents, conditions, cycles, etc.) of each of which is independently incorporated herein by reference. In some embodiments, an internucleotidic linkage is described in WO 2012/030683, WO 2021/030778, WO 2019112485, US 20170362270, WO 2018156056, WO 2018056871, WO 2020/154344, WO 2020/154343, WO 2020/154342, WO 2020/165077, WO 2020/201406, WO 2020/216637, or WO 2020/252376, and can be utilized in accordance with the present disclosure.

In some embodiments, each internucleotidic linkage in an oligonucleotide is independently selected from a natural phosphate linkage, a phosphorothioate linkage, and a non-negatively charged internucleotidic linkage (e.g., n001, n003, n004, n006, n008, n009, or n013). In some embodiments, each internucleotidic linkage in an oligonucleotide is independently selected from a natural phosphate linkage, a phosphorothioate linkage, and a neutral internucleotidic linkage (e.g., n001, n003, n004, n006, n008, n009, or n013). In some embodiments, an oligonucleotide comprises an internucleotidic linkage selected from n001, n002, n003, n004, n006, n008, n009, n012, n013 n020, n021, n024, n025, n026, n029, n030, n031, n033, n034, n035, n036, n037, n041, n043, n044, n046, n047, n048, n051, n052, n054, n055, and n057.

In some embodiments, an oligonucleotide comprises one or more (e.g., 1-20, 1-15, 1-10, 1-8, 1-5, 1-4, 1-3, 1, 2, 3, 4, 5, 6, 7, 8, 9, or 10) non-negatively charged internucleotidic linkage. In some embodiments, an oligonucleotide contains no more than 10 non-negatively charged internucleotidic linkages. In some embodiments, an oligonucleotide contains no more than 9 non-negatively charged internucleotidic linkages. In some embodiments, an oligonucleotide contains no more than 8 non-negatively charged internucleotidic linkages. In some embodiments, an oligonucleotide contains no more than 7 non-negatively charged internucleotidic linkages. In some embodiments, an oligonucleotide contains no more than 6 non-negatively charged internucleotidic linkages. In some embodiments, an oligonucleotide contains no more than 5 non-negatively charged internucleotidic linkages. In some embodiments, an oligonucleotide contains no more than 4 non-negatively charged internucleotidic linkages. In some embodiments, an oligonucleotide contains no more than 3 non-negatively charged internucleotidic linkages. In some embodiments, an oligonucleotide contains no more than 10 consecutive non-negatively charged internucleotidic linkages. In some embodiments, an oligonucleotide contains no more than 9 consecutive non-negatively charged internucleotidic linkages. In some embodiments, an oligonucleotide contains no more than 8 consecutive non-negatively charged internucleotidic linkages. In some embodiments, an oligonucleotide contains no more than 7 consecutive non-negatively charged internucleotidic linkages. In some embodiments, an oligonucleotide contains no more than 6 consecutive non-negatively charged internucleotidic linkages. In some embodiments, an oligonucleotide contains no more than 5 consecutive non-negatively charged internucleotidic linkages. In some embodiments, an oligonucleotide contains no more than 4 consecutive non-negatively charged internucleotidic linkages. In some embodiments, an oligonucleotide contains no more than 3 consecutive non-negatively charged internucleotidic linkages. In some embodiments, an oligonucleotide comprises 2 or more consecutive non-negatively charged internucleotidic linkages. In some embodiments, an oligonucleotide comprises 3 or more consecutive non-negatively charged internucleotidic linkages. In some embodiments, an oligonucleotide comprises 4 or more consecutive non-negatively charged internucleotidic linkages. In some embodiments, an oligonucleotide comprises 5 or more consecutive non-negatively charged internucleotidic linkages. In some embodiments, one or more or all non-negatively charged internucleotidic linkages are in wings of an oligonucleotide comprising wing-core-wing. In some embodiments, one or more or all non-negatively charged internucleotidic linkages are in a wing of an oligonucleotide comprising wing-core-wing. In some embodiments, one or more or all non-negatively charged internucleotidic linkages are in a core of an oligonucleotide comprising wing-core-wing. In some embodiments, each non-negatively charged internucleotidic linkages is in a wing. In some embodiments, each non-negatively charged internucleotidic linkages is in the same wing. In some embodiments, each non-negatively charged internucleotidic linkages is in a core. In some embodiments, one or more non-negatively charged internucleotidic linkages are in wings, and one or more non-negatively charged internucleotidic linkages are in a core. In some embodiments, each non-negatively charged internucleotidic linkage is independently a neutral internucleotidic linkage. In some embodiments, each non-negatively charged internucleotidic linkage independently comprises a linkage phosphorus bonded to a nitrogen atom which connects a sugar to a linkage. In some embodiments, each non-negatively charged internucleotidic linkage independently comprises —NR′ —N═C[N(R′)₂]₂, or —N═C(-L^{L}-R′)₂. In some embodiments, one or more (e.g., about or at least about 1, 2, 3, 4, 5, 6, 7, 8, 9, or 10) internucleotidic linkages are independently n001. In some embodiments, one or more (e.g., about or at least about 1, 2, 3, 4, 5, 6, 7, 8, 9, or 10) internucleotidic linkages are independently n003. In some embodiments, one or more (e.g., about or at least about 1, 2, 3, 4, 5, 6, 7, 8, 9, or 10) internucleotidic linkages are independently n004. In some embodiments, one or more (e.g., about or at least about 1, 2, 3, 4, 5, 6, 7, 8, 9, or 10) internucleotidic linkages are independently n008. In some embodiments, one or more (e.g., about or at least about 1, 2, 3, 4, 5, 6, 7, 8, 9, or 10) internucleotidic linkages are independently n025. In some embodiments, one or more (e.g., about or at least about 1, 2, 3, 4, 5, 6, 7, 8, 9, or 10) internucleotidic linkages are independently n026. In some embodiments, one or more (e.g., about or at least about 1, 2, 3, 4, 5, 6, 7, 8, 9, or 10) internucleotidic linkages are independently n029. In some embodiments, each non-negatively charged internucleotidic linkage is independently selected from n001, n003, n004, n008, n025, n026, n029, n030, n031, n033, n036, and n037. In some embodiments, each non-negatively charged internucleotidic linkage is independently selected from n001, n003, n004, n025, n026, and n029. In some embodiments, one or more non-negatively charged internucleotidic linkage is n001, and one or more internucleotidic linkage is selected from n003, n004, n008, n025, n026, n029, n030, n031, n033, n036, and n037. In some embodiments, each non-negatively charged internucleotidic linkage is the same. In some embodiments, each non-negatively charged internucleotidic linkage is independently n001. In some embodiments, each non-negatively charged internucleotidic linkage is independently n003. In some embodiments, each non-negatively charged internucleotidic linkage is independently n004. In some embodiments, each non-negatively charged internucleotidic linkage is independently n008. In some embodiments, each non-negatively charged internucleotidic linkage is independently n025. In some embodiments, each non-negatively charged internucleotidic linkage is independently n026. In some embodiments, each non-negatively charged internucleotidic linkage is independently n029.

In some embodiments, an oligonucleotide comprises one or more (e.g., 1-20, 1-15, 1-10, 1-8, 1-5, 1-4, 1-3, 1, 2, 3, 4, 5, 6, 7, 8, 9, or 10) n001. In some embodiments, an oligonucleotide contains no more than 10 n001. In some embodiments, an oligonucleotide contains no more than 9 n001. In some embodiments, an oligonucleotide contains no more than 8 n001. In some embodiments, an oligonucleotide contains no more than 7 n001. In some embodiments, an oligonucleotide contains no more than 6 n001. In some embodiments, an oligonucleotide contains no more than 5 n001. In some embodiments, an oligonucleotide contains no more than 4 n001. In some embodiments, an oligonucleotide contains no more than 3 n001. In some embodiments, an oligonucleotide contains no more than 10 consecutive n001. In some embodiments, an oligonucleotide contains no more than 9 consecutive n001. In some embodiments, an oligonucleotide contains no more than 8 consecutive n001. In some embodiments, an oligonucleotide contains no more than 7 consecutive n001. In some embodiments, an oligonucleotide contains no more than 6 consecutive n001. In some embodiments, an oligonucleotide contains no more than 5 consecutive n001. In some embodiments, an oligonucleotide contains no more than 4 consecutive n001. In some embodiments, an oligonucleotide contains no more than 3 consecutive n001. In some embodiments, an oligonucleotide comprises 2 or more consecutive n001. In some embodiments, an oligonucleotide comprises 3 or more consecutive n001. In some embodiments, an oligonucleotide comprises 4 or more consecutive n001. In some embodiments, an oligonucleotide comprises 5 or more consecutive n001. In some embodiments, one or more or all n001 are in wings of an oligonucleotide comprising wing-core-wing. In some embodiments, one or more or all n001 are in a wing of an oligonucleotide comprising wing-core-wing. In some embodiments, one or more or all n001 are in a core of an oligonucleotide comprising wing-core-wing. In some embodiments, each n001 is in a wing. In some embodiments, each n001 is in the same wing. In some embodiments, each n001 is in a core. In some embodiments, one or more n001 are in wings, and one or more n001 are in a core.

In some embodiments, an oligonucleotide comprises one or more (e.g., 1-20, 1-15, 1-10, 1-8, 1-5, 1-4, 1-3, 1, 2, 3, 4, 5, 6, 7, 8, 9, or 10) n013. In some embodiments, an oligonucleotide contains no more than 10 n013. In some embodiments, an oligonucleotide contains no more than 9 n013. In some embodiments, an oligonucleotide contains no more than 8 n013. In some embodiments, an oligonucleotide contains no more than 7 n013. In some embodiments, an oligonucleotide contains no more than 6 n013. In some embodiments, an oligonucleotide contains no more than 5 n013. In some embodiments, an oligonucleotide contains no more than 4 n013. In some embodiments, an oligonucleotide contains no more than 3 n013. In some embodiments, an oligonucleotide contains no more than 10 consecutive n013. In some embodiments, an oligonucleotide contains no more than 9 consecutive n013. In some embodiments, an oligonucleotide contains no more than 8 consecutive n013. In some embodiments, an oligonucleotide contains no more than 7 consecutive n013. In some embodiments, an oligonucleotide contains no more than 6 consecutive n013. In some embodiments, an oligonucleotide contains no more than 5 consecutive n013. In some embodiments, an oligonucleotide contains no more than 4 consecutive n013. In some embodiments, an oligonucleotide contains no more than 3 consecutive n013. In some embodiments, one or more n013 are each independently bonded to a nitrogen atom (e.g., of sm01 as in sm01n013). In some embodiments, each n013 is independently bonded to a nitrogen atom (e.g., of sm01 as in sm01n013). As confirmed in the Examples, various compositions of oligonucleotides comprising n013 can provide desired activities. In some embodiments, one or more or all n013 are in wings of an oligonucleotide comprising wing-core-wing. In some embodiments, one or more or all n013 are in a wing of an oligonucleotide comprising wing-core-wing. In some embodiments, one or more or all n013 are in a core of an oligonucleotide comprising wing-core-wing. In some embodiments, each n013 is in a wing. In some embodiments, each n013 is in the same wing. In some embodiments, each n013 is in a core. In some embodiments, one or more n013 are in wings, and one or more n013 are in a core.

In some embodiments, a linkage is or comprises —CH₂C(O)NR′—, wherein the —CH₂— is optionally substituted. In some embodiments, R′ is H. In some embodiments, —NR′— is connected to a 3′ side sugar.

In some embodiments, a linkage is or comprises

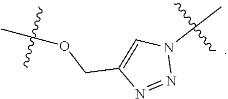 [SMILES: *OCc1cn(*)nn1]

In some embodiments, the —O— is connected to a 5′ side sugar. In some embodiments, a linkage is or comprises

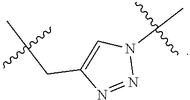 [SMILES: *Cc1cn(*)nn1]

In some embodiments, —CH₂— is connected to a 5′ side sugar.

Oligonucleotides can comprise various numbers of natural phosphate linkages, e.g., 1-50, 1-40, 1-30, 1-25, 1-20, 1-10, 1-5, or 1, 2, 3, 4, 5, 6, 7, 8, 9, 10, 11, 12, 13, 14, 15, 16, 17, 18, 19, 20 or more. In some embodiments, one or more (e.g., 1-50, 1-40, 1-30, 1-25, 1-20, 1-10, 1-5, or 1, 2, 3, 4, 5, 6, 7, 8, 9, 10, 11, 12, 13, 14, 15, 16, 17, 18, 19, 20 or more) of the natural phosphate linkages in an oligonucleotide are consecutive. In some embodiments, provided oligonucleotides comprise no natural phosphate linkages. In some embodiments, provided oligonucleotides comprise one natural phosphate linkage. In some embodiments, provided oligonucleotides comprise 1 to 30 or more natural phosphate linkages.

In some embodiments, a modified internucleotidic linkage is a chiral internucleotidic linkage which comprises a chiral linkage phosphorus. In some embodiments, a chiral internucleotidic linkage is a phosphorothioate linkage. In some embodiments, a chiral internucleotidic linkage is a non-negatively charged internucleotidic linkage. In some embodiments, a chiral internucleotidic linkage is a neutral internucleotidic linkage. In some embodiments, a chiral internucleotidic linkage is chirally controlled with respect to its chiral linkage phosphorus. In some embodiments, a chiral internucleotidic linkage is stereochemically pure with respect to its chiral linkage phosphorus. In some embodiments, a chiral internucleotidic linkage is not chirally controlled. In some embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

In some embodiments, provided oligonucleotides comprise one or more non-negatively charged internucleotidic linkages. In some embodiments, provided oligonucleotides comprise one or more neutral internucleotidic linkages. In some embodiments, provided oligonucleotides comprise one or more phosphoryl guanidine internucleotidic linkages. In some embodiments, a neutral internucleotidic linkage or non-negatively charged internucleotidic linkage is a phosphoryl guanidine internucleotidic linkage. In some embodiments, each neutral internucleotidic linkage or non-negatively charged internucleotidic linkage is independently a phosphoryl guanidine internucleotidic linkage. In some embodiments, each neutral internucleotidic linkage and non-negatively charged internucleotidic linkage is independently n001.

In some embodiments, each internucleotidic linkage in a provided oligonucleotide is independently selected from a phosphorothioate internucleotidic linkage, a phosphoryl guanidine internucleotidic linkage, and a natural phosphate linkage. In some embodiments, each internucleotidic linkage in a provided oligonucleotide is independently selected from a phosphorothioate internucleotidic linkage, n001, and a natural phosphate linkage.

Various types of internucleotidic linkages may be utilized in combination of other structural elements, e.g., sugars, to achieve desired oligonucleotide properties and/or activities. For example, the present disclosure routinely utilizes modified internucleotidic linkages and modified sugars, optionally with natural phosphate linkages and natural sugars, in designed oligonucleotides. In some embodiments, the present disclosure provides an oligonucleotide comprising one or more modified sugars. In some embodiments, the present disclosure provides an oligonucleotide comprising one or more modified sugars and one or more modified internucleotidic linkages, one or more of which are natural phosphate linkages.

In some embodiments, an internucleotidic linkage is a phosphoryl guanidine, phosphoryl amidine, phosphoryl isourea, phosphoryl isothiourea, phosphoryl imidate, or phosphoryl imidothioate internucleotidic linkage, e.g., those as described in US 20170362270.

In some embodiments, stability of various internucleotidic linkages is assessed. In some embodiments, internucleotidic linkages are exposed to various conditions utilized for oligonucleotide manufacturing, e.g., solid phase oligonucleotide synthesis, including reagents, solvents, temperatures (in some cases, temperatures higher than room temperature), cleavage conditions, deprotection conditions, purification conditions, etc., and stability is assessed. In some embodiments, stable internucleotidic linkages (e.g., those having no more than 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% degradation when exposed to one or more conditions and/or processes, or after a complete oligonucleotide manufacturing process) are selected for utilization in various oligonucleotide compositions and applications.

As described herein, various variables can be R, e.g., R′, R^{L}, etc. Various embodiments for R are described in the present disclosure (e.g., when describing variables that can be R). Such embodiments are generally useful for all variables that can be R. In some embodiments, R is hydrogen. In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) aliphatic. In some embodiments, R is optionally substituted C₁₋₂₀ aliphatic. In some embodiments, R is optionally substituted C₁₋₁₀ aliphatic. In some embodiments, R is optionally substituted C₁₋₆ aliphatic. In some embodiments, R is optionally substituted alkyl. In some embodiments, R is optionally substituted C₁₋₆ alkyl. In some embodiments, R is optionally substituted methyl. In some embodiments, R is methyl. In some embodiments, R is optionally substituted ethyl. In some embodiments, R is optionally substituted propyl. In some embodiments, R is isopropyl. In some embodiments, R is optionally substituted butyl. In some embodiments, R is optionally substituted pentyl. In some embodiments, R is optionally substituted hexyl.

In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) cycloaliphatic. In some embodiments, R is optionally substituted cycloalkyl. In some embodiments, cycloaliphatic is monocyclic, bicyclic, or polycyclic, wherein each monocyclic unit is independently saturated or partially saturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted adamantyl.

In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) heteroaliphatic having 1-10 heteroatoms. In some embodiments, R is optionally substituted C₁₋₂₀ aliphatic having 1-10 heteroatoms. In some embodiments, R is optionally substituted C₁₋₁₀ aliphatic having 1-10 heteroatoms. In some embodiments, R is optionally substituted C₁₋₆ aliphatic having 1-3 heteroatoms. In some embodiments, R is optionally substituted heteroalkyl. In some embodiments, R is optionally substituted C₁₋₆ heteroalkyl. In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) heterocycloaliphatic having 1-10 heteroatoms. In some embodiments, R is optionally substituted heteroclycloalkyl. In some embodiments, heterocycloaliphatic is monocyclic, bicyclic, or polycyclic, wherein each monocyclic unit is independently saturated or partially saturated.

In some embodiments, R is optionally substituted C₆₋₃₀ aryl. In some embodiments, R is optionally substituted phenyl. In some embodiments, R is optionally substituted phenyl. In some embodiments, R is C₆₋₁₄ aryl. In some embodiments, R is optionally substituted bicyclic aryl. In some embodiments, R is optionally substituted polycyclic aryl. In some embodiments, R is optionally substituted C₆₋₃₀ arylaliphatic. In some embodiments, R is C₆₋₃₀ arylheteroaliphatic having 1-10 heteroatoms.

In some embodiments, R is optionally substituted 5-30 (5, 6, 7, 8, 9, 10, 11, 12, 13, 14, 15, 16, 17, 18, 19, 20, 21, 22, 23, 24, 25, 26, 27, 28, 29 or 30) membered heteroaryl having 1-10 heteroatoms. In some embodiments, R is optionally substituted 5-20 membered heteroaryl having 1-10 heteroatoms. In some embodiments, R is optionally substituted 5-10 membered heteroaryl having 1-10 heteroatoms. In some embodiments, R is optionally substituted 5-membered heteroaryl having 1-5 heteroatoms. In some embodiments, R is optionally substituted 5-membered heteroaryl having 1-4 heteroatoms. In some embodiments, R is optionally substituted 5-membered heteroaryl having 1-3 heteroatoms. In some embodiments, R is optionally substituted 5-membered heteroaryl having 1-2 heteroatoms. In some embodiments, R is optionally substituted 5-membered heteroaryl having one heteroatom. In some embodiments, R is optionally substituted 6-membered heteroaryl having 1-5 heteroatoms. In some embodiments, R is optionally substituted 6-membered heteroaryl having 1-4 heteroatoms. In some embodiments, R is optionally substituted 6-membered heteroaryl having 1-3 heteroatoms. In some embodiments, R is optionally substituted 6-membered heteroaryl having 1-2 heteroatoms. In some embodiments, R is optionally substituted 6-membered heteroaryl having one heteroatom. In some embodiments, R is optionally substituted monocyclic heteroaryl. In some embodiments, R is optionally substituted bicyclic heteroaryl. In some embodiments, R is optionally substituted polycyclic heteroaryl. In some embodiments, a heteroatom is nitrogen.

In some embodiments, R is optionally substituted 2-pyridinyl. In some embodiments, R is optionally substituted 3-pyridinyl. In some embodiments, R is optionally substituted 4-pyridinyl. In some embodiments, R is optionally substituted

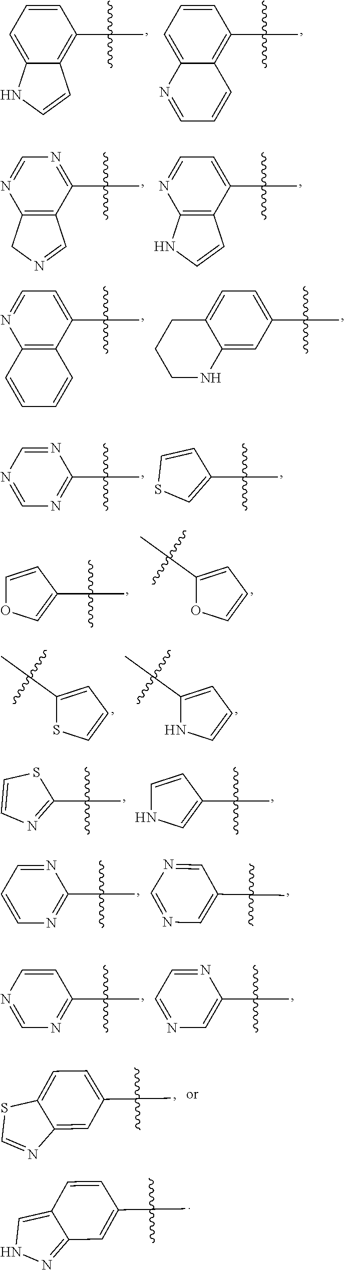

In some embodiments, R is optionally substituted 3-30 (3, 4, 5, 6, 7, 8, 9, 10, 11, 12, 13, 14, 15, 16, 17, 18, 19, 20, 21, 22, 23, 24, 25, 26, 27, 28, 29 or 30) membered heterocyclyl having 1-10 heteroatoms. In some embodiments, R is optionally substituted 3-membered heterocyclyl having 1-2 heteroatoms. In some embodiments, R is optionally substituted 4-membered heterocyclyl having 1-2 heteroatoms. In some embodiments, R is optionally substituted 5-20 membered heterocyclyl having 1-10 heteroatoms. In some embodiments, R is optionally substituted 5-10 membered heterocyclyl having 1-10 heteroatoms. In some embodiments, R is optionally substituted 5-membered heterocyclyl having 1-5 heteroatoms. In some embodiments, R is optionally substituted 5-membered heterocyclyl having 1-4 heteroatoms. In some embodiments, R is optionally substituted 5-membered heterocyclyl having 1-3 heteroatoms. In some embodiments, R is optionally substituted 5-membered heterocyclyl having 1-2 heteroatoms. In some embodiments, R is optionally substituted 5-membered heterocyclyl having one heteroatom. In some embodiments, R is optionally substituted 6-membered heterocyclyl having 1-5 heteroatoms. In some embodiments, R is optionally substituted 6-membered heterocyclyl having 1-4 heteroatoms. In some embodiments, R is optionally substituted 6-membered heterocyclyl having 1-3 heteroatoms. In some embodiments, R is optionally substituted 6-membered heterocyclyl having 1-2 heteroatoms. In some embodiments, R is optionally substituted 6-membered heterocyclyl having one heteroatom. In some embodiments, R is optionally substituted monocyclic heterocyclyl. In some embodiments, R is optionally substituted bicyclic heterocyclyl. In some embodiments, R is optionally substituted polycyclic heterocyclyl. In some embodiments, R is optionally substituted saturated heterocyclyl. In some embodiments, R is optionally substituted partially unsaturated heterocyclyl. In some embodiments, a heteroatom is nitrogen. In some embodiments, R is optionally substituted

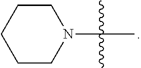 [SMILES: *N1CCCCC1]

In some embodiments, R is optionally substituted

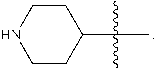 [SMILES: *C1CCNCC1]

In some embodiments, R is optionally substituted

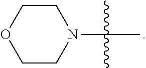 [SMILES: *N1CCOCC1]

In some embodiments, two R groups are optionally and independently taken together to form a covalent bond. In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms. In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

Various variables may comprises an optionally substituted ring, or can be taken together with their intervening atom(s) to form a ring. In some embodiments, a ring is 3-30 (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) membered. In some embodiments, a ring is 3-20 membered. In some embodiments, a ring is 3-15 membered. In some embodiments, a ring is 3-10 membered. In some embodiments, a ring is 3-8 membered. In some embodiments, a ring is 3-7 membered. In some embodiments, a ring is 3-6 membered. In some embodiments, a ring is 4-20 membered. In some embodiments, a ring is 5-20 membered. In some embodiments, a ring is monocyclic. In some embodiments, a ring is bicyclic. In some embodiments, a ring is polycyclic. In some embodiments, each monocyclic ring or each monocyclic ring unit in bicyclic or polycyclic rings is independently saturated, partially saturated or aromatic. In some embodiments, each monocyclic ring or each monocyclic ring unit in bicyclic or polycyclic rings is independently 3-10 membered and has 0-5 heteroatoms.

In some embodiments, each heteroatom is independently selected oxygen, nitrogen, sulfur, silicon, and phosphorus. In some embodiments, each heteroatom is independently selected oxygen, nitrogen, sulfur, and phosphorus. In some embodiments, each heteroatom is independently selected oxygen, nitrogen, and sulfur. In some embodiments, a heteroatom is in an oxidized form.

### Nucleobases

Various nucleobases may be utilized in provided oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5 mC, 5-hydroxymethyl C, etc. In some embodiments, a nucleobase is alkyl-substituted A, T, C, G or U. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5 mC. In some embodiments, a nucleobase is substituted A, T, C, G or U. In some embodiments, a nucleobase is a substituted tautomer of A, T, C, G or U. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5 mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5 mC may be treated the same as C [e.g., an oligonucleotide having 5 mC in place of C (e.g., AT5mCG) is considered to have the same base sequence as an oligonucleotide having C at the corresponding location(s) (e.g., ATCG)].

In some embodiments, an oligonucleotide comprises one or more A, T, C, G or U. In some embodiments, an oligonucleotide comprises one or more optionally substituted A, T, C, G or U. In some embodiments, an oligonucleotide comprises one or more 5-methylcytidine, 5-hydroxymethylcytidine, 5-formylcytosine, or 5-carboxylcytosine. In some embodiments, an oligonucleotide comprises one or more 5-methylcytidine. In some embodiments, each nucleobase in an oligonucleotide is selected from the group consisting of optionally substituted A, T, C, G and U, and optionally substituted tautomers of A, T, C, G and U. In some embodiments, each nucleobase in an oligonucleotide is optionally protected A, T, C, G and U. In some embodiments, each nucleobase in an oligonucleotide is optionally substituted A, T, C, G or U. In some embodiments, each nucleobase in an oligonucleotide is selected from the group consisting of A, T, C, G, U, and 5 mC.

In some embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, RNA, 2003, 9, 1034-1048, Limbach et al. Nucleic Acids Research, 1994, 22, 2183-2196 and Revankar and Rao, Comprehensive Natural Products Chemistry, vol. 7, 313. In some embodiments, a modified nucleobase is substituted uracil, thymine, adenine, cytosine, or guanine. In some embodiments, a modified nucleobase is a functional replacement, e.g., in terms of hydrogen bonding and/or base pairing, of uracil, thymine, adenine, cytosine, or guanine. In some embodiments, a nucleobase is optionally substituted uracil, thymine, adenine, cytosine, 5-methylcytosine, or guanine. In some embodiments, a nucleobase is uracil, thymine, adenine, cytosine, 5-methylcytosine, or guanine.

In some embodiments, a provided oligonucleotide comprises one or more 5-methylcytosine. In some embodiments, the present disclosure provides an oligonucleotide whose base sequence is disclosed herein, e.g., in Table A1, A2, A3, and A4, wherein each T may be independently replaced with U and vice versa, and each cytosine is optionally and independently replaced with 5-methylcytosine or vice versa. As appreciated by those skilled in the art, in some embodiments, 5 mC may be treated as C with respect to base sequence of an oligonucleotide - such oligonucleotide comprises a nucleobase modification at the C position (e.g., see various oligonucleotides in Table A1, A2, A3, and A4). In description of oligonucleotides, typically unless otherwise noted, nucleobases, sugars and internucleotidic linkages are non-modified.

In some embodiments, a modified base is optionally substituted adenine, cytosine, guanine, thymine, or uracil, or a tautomer thereof. In some embodiments, a modified nucleobase is a modified adenine, cytosine, guanine, thymine or uracil, modified by one or more modifications by which:

(1) a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof;

(2) one or more atoms of a nucleobase are independently replaced with a different atom selected from carbon, nitrogen and sulfur;

(3) one or more double bonds in a nucleobase are independently hydrogenated; or

(4) one or more aryl or heteroaryl rings are independently inserted into a nucleobase.

In some embodiments, a modified nucleobase is a modified nucleobase known in the art, e.g., WO2017/210647. In some embodiments, modified nucleobases are expanded-size nucleobases in which one or more aryl and/or heteroaryl rings, such as phenyl rings, have been added.

In some embodiments, a modified nucleobase is selected from 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from 2-aminopropyladenine, 5-hydroxymethyl cytosine, xanthine, hypoxanthine, 2-aminoadenine, 6-N-methylguanine, 6-N- methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4- thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N- benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. In some embodiments, modified nucleobases are tricyclic pyrimidines, such as 1,3-diazaphenoxazine-2-one, 1,3-diazaphenothiazine-2-one or 9-(2-aminoethoxy)-1,3-diazaphenoxazine-2- one (G-clamp). In some embodiments, modified nucleobases are those in which the purine or pyrimidine base is replaced with other heterocycles, for example, 7-deaza-adenine, 7-deazaguanosine, 2-aminopyridine or 2-pyridone.

In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One example of a universal base is 3-nitropyrrole.

In some embodiments, nucleosides that can be utilized in provided technologies comprise modified nucleobases and/or modified sugars, e.g., 4-acetylcytidine; 5-(carboxyhydroxylmethypuridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine ; 5-methyluridine ; 2′-O-methyl-5-methyluridine; and 2′-O-methyluridine.

In some embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5-iodouracil, or 2,6-diaminopurine. In some embodiments, a nucleobase comprises substitution with a fluorescent or biomolecule binding moiety. In some embodiments, a substituent is a fluorescent moiety. In some embodiments, a substituent is biotin or avidin.

In some embodiments, in various formulae, BA is a nucleobase as described herein. In some embodiments, BA is an optionally substituted group selected from C₃₋₃₀ cycloaliphatic, C₆₋₃₀ aryl, C₅₋₃₀ heteroaryl having 1-10 heteroatoms, C₃₋₃₀ heterocyclyl having 1-10 heteroatoms, a natural nucleobase moiety, and a modified nucleobase moiety. In some embodiments, BA is an optionally substituted, saturated, partially unsaturated or aromatic C₃₋₃₀ monocyclic, bicyclic or polycyclic ring having 0-10 heteroatoms. In some embodiments, each monocyclic wring in BA is optionally substituted 3-10 membered saturated, partially unsaturated or aromatic ring having 1-5 heteroatoms. In some embodiments, one or more ring heteroatom is nitrogen. In some embodiments, BA comprises one or more partially unsaturated monocyclic rings. In some embodiments, BA comprises one or more aromatic rings. In some embodiments, BA comprises one or more heteroaryl rings. In some embodiments, BA comprises one or more heteroaryl rings, one or more of which independently comprise a nitrogen atom. In some embodiments, BA comprises one or more heterocyclyl rings, one or more of which independently comprise a nitrogen atom. In some embodiments, a ring, e.g., a monocyclic ring unit in BA, or BA, is 5-membered. In some embodiments, a monocyclic ring unit in BA, or BA, is 6-membered. In some embodiments, a bicyclic ring unit in BA, or BA, is 8-10-membered. In some embodiments, it is 8-membered. In some embodiments, it is 9-membered. In some embodiments, it is 10-membered.

In some embodiments, a nucleobase, e.g., BA, comprises at least one optionally substituted ring which comprises a heteroatom ring atom. In some embodiments, a nucleobase comprises at least one optionally substituted ring which comprises a nitrogen ring atom. In some embodiments, such a ring is aromatic. In some embodiments, a nucleobase is bonded to a sugar through a heteroatom. In some embodiments, a nucleobase is bonded to a sugar through a nitrogen atom. In some embodiments, a nucleobase is bonded to a sugar through a ring nitrogen atom.

In some embodiments, a nucleobase, e.g., BA, is one described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the nucleobases of each of which are incorporated herein by reference.

For example, in some embodiments, a nucleobase, e.g., BA, is an optionally substituted group, which group is formed by removing a —H from

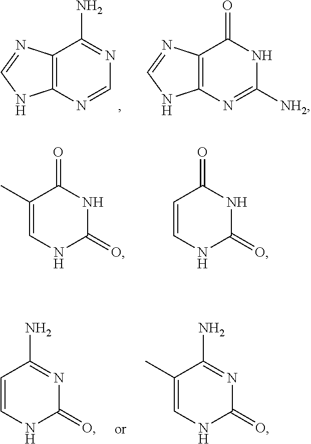

or a tautomer thereof. In some embodiments, a nucleobase, e.g., BA, is an optionally substituted group, which group is formed by removing a —H from

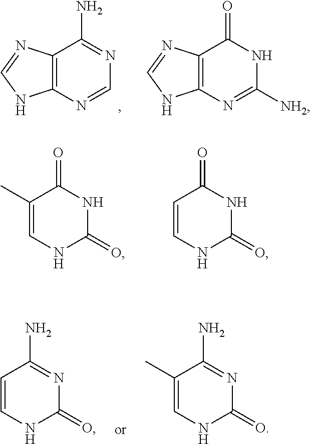

In some embodiments, a nucleobase, e.g., BA, is an optionally substituted group which group is selected from

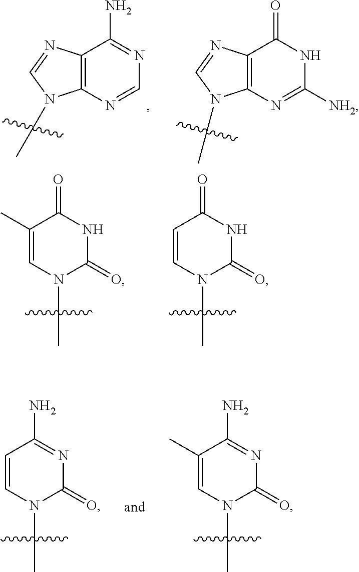

and tautomeric forms thereof. In some embodiments, a nucleobase, e.g., BA, is an optionally substituted group which group is selected from

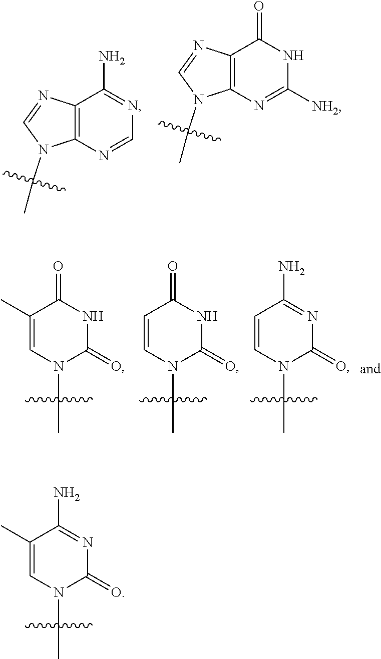

In some embodiments, a nucleobase, e.g., BA, is an optionally substituted group, which group is formed by removing a —H from

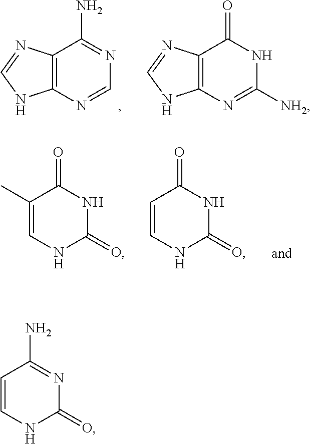

and tautomers thereof. In some embodiments, a nucleobase, e.g., BA, is an optionally substituted group, which group is formed by removing a —H from

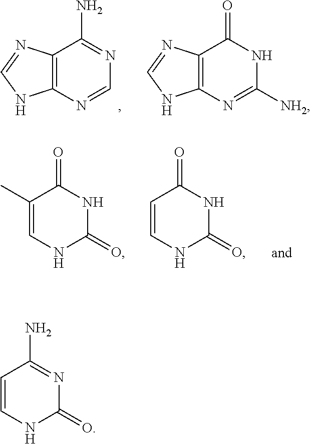

In some embodiments, a nucleobase, e.g., BA, is an optionally substituted group which group is selected from

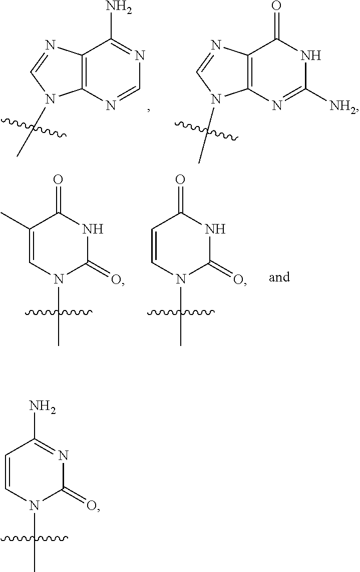

and tautomeric forms thereof. In some embodiments, a nucleobase, e.g., BA, is an optionally substituted group which group is selected from

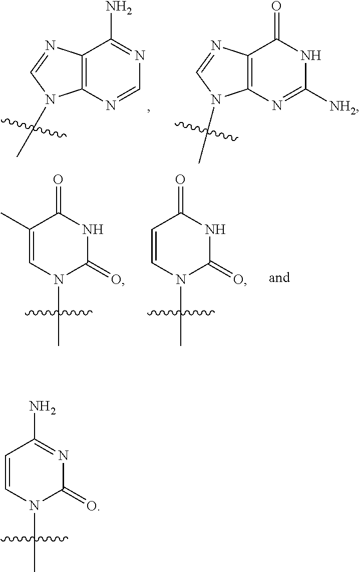

In some embodiments, a nucleobase, e.g., BA is optionally substituted

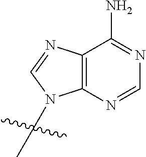 [SMILES: *n1cnc2c(N)ncnc21]

or a tautomeric form thereof. In some embodiments, a nucleobase, e.g., BA is optionally substituted

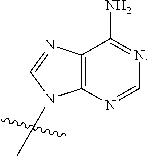 [SMILES: *n1cnc2c(N)ncnc21]

In some embodiments, a nucleobase, e.g., BA is optionally substituted

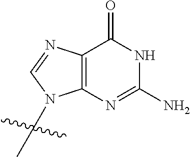 [SMILES: *n1cnc2c(=O)[nH]c(N)nc21]

or a tautomeric form thereof. In some embodiments, a nucleobase, e.g., BA is optionally substituted

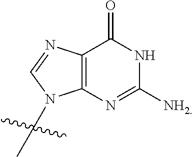 [SMILES: *n1cnc2c(=O)[nH]c(N)nc21]

In some embodiments, a nucleobase, e.g., BA is optionally substituted

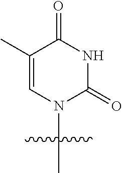 [SMILES: *n1cc(C)c(=O)[nH]c1=O]

or a tautomeric form thereof. In some embodiments, a nucleobase, e.g., BA is optionally substituted

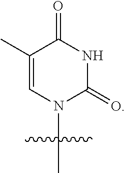 [SMILES: *n1cc(C)c(=O)[nH]c1=O]

In some embodiments, a nucleobase, e.g., BA is optionally substituted

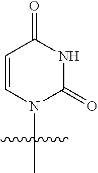 [SMILES: *n1ccc(=O)[nH]c1=O]

or a tautomeric form thereof. In some embodiments, a nucleobase, e.g., BA is optionally substituted

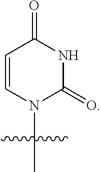 [SMILES: *n1ccc(=O)[nH]c1=O]

In some embodiments, a nucleobase, e.g., BA is optionally substituted

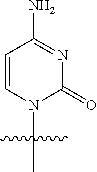 [SMILES: *n1ccc(N)nc1=O]

or a tautomeric form thereof. In some embodiments, a nucleobase, e.g., BA is optionally substituted

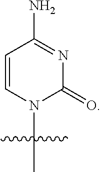 [SMILES: *n1ccc(N)nc1=O]

In some embodiments, a nucleobase, e.g., BA is

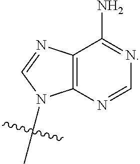 [SMILES: *n1cnc2c(N)ncnc21]

In some embodiments, a nucleobase, e.g., BA is

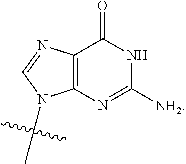 [SMILES: *n1cnc2c(=O)[nH]c(N)nc21]

In some embodiments, a nucleobase, e.g., BA is

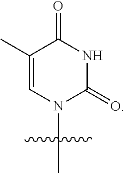 [SMILES: *n1cc(C)c(=O)[nH]c1=O]

In some embodiments, a nucleobase, e.g., BA is

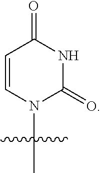 [SMILES: *n1ccc(=O)[nH]c1=O]

In some embodiments, a nucleobase, e.g., BA is

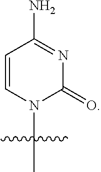 [SMILES: *n1ccc(N)nc1=O]

In some embodiments, a nucleobase, e.g., BA, is

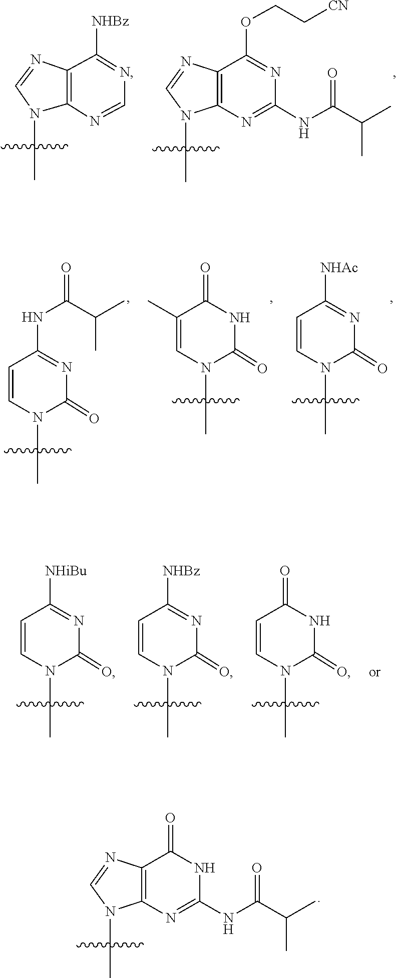

In some embodiments, a nucleobase, e.g., BA, is

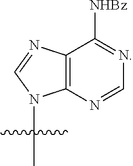

In some embodiments, a nucleobase, e.g., BA, is

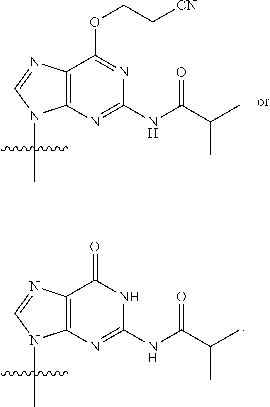 [SMILES: *n1cnc2c(OCCC#N)nc(NC(=O)C(C)C)nc21 ; *n1cnc2c(=O)[nH]c(NC(=O)C(C)C)nc21]

In some embodiments, a nucleobase, e.g., BA, is

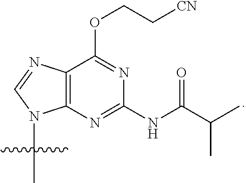 [SMILES: *n1cnc2c(OCCC#N)nc(NC(=O)C(C)C)nc21]

In some embodiments, a nucleobase, e.g., BA, is

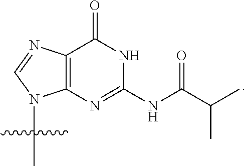 [SMILES: *n1cnc2c(=O)[nH]c(NC(=O)C(C)C)nc21]

In some embodiments, a nucleobase, e.g., BA, is

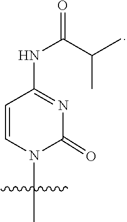 [SMILES: *n1ccc(NC(=O)C(C)C)nc1=O]

In some embodiments, a nucleobase, e.g., BA, is

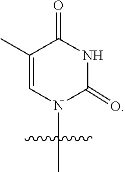 [SMILES: *n1cc(C)c(=O)[nH]c1=O]

In some embodiments, a nucleobase, e.g., BA, is

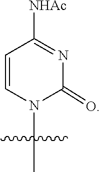 [SMILES: *n1ccc(NC(C)=O)nc1=O]

In some embodiments, a nucleobase, e.g., BA, is

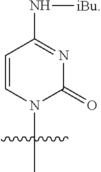

In some embodiments, a nucleobase, e.g., BA, is

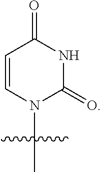 [SMILES: *n1ccc(=O)[nH]c1=O]

In some embodiments, a protection group is —Ac. In some embodiments, a protection group is —Bz. In some embodiments, a protection group is -iBu for nucleobase.

In some embodiments, a nucleobase, e.g., BA, is optionally substituted hypoxanthine or a tautomer thereof.

In some embodiments, a nucleobase, e.g., BA, is an optionally substituted purine base residue. In some embodiments, a nucleobase is a protected purine base residue. In some embodiments, a nucleobase is an optionally substituted adenine residue. In some embodiments, a nucleobase is a protected adenine residue. In some embodiments, a nucleobase is an optionally substituted guanine residue. In some embodiments, a nucleobase is a protected guanine residue. In some embodiments, a nucleobase is an optionally substituted cytosine residue. In some embodiments, a nucleobase is a protected cytosine residue. In some embodiments, a nucleobase is an optionally substituted thymine residue. In some embodiments, a nucleobase is a protected thymine residue. In some embodiments, a nucleobase is an optionally substituted uracil residue. In some embodiments, a nucleobase is a protected uracil residue. In some embodiments, a nucleobase is an optionally substituted 5-methylcytosine residue. In some embodiments, a nucleobase is a protected 5-methylcytosine residue.

In some embodiments, an oligonucleotide comprises a nucleobase or modified nucleobase as described in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the bases and modified nucleobases of each of which are independently incorporated herein by reference.

In some embodiments, a provided oligonucleotide comprises a modified nucleobase described in, e.g., U.S. Pat. Nos. 5,552,540, 6,222,025, 6,528,640, 4,845,205, 5,681,941, 5,750,692, 6,015,886, 5,614,617, 6,147,200, 5,457,187, 6,639,062, 7,427,672, 5,459,255, 5,484,908, 7,045,610, 3,687,808, 5,502,177, 5,525,711 6,235,887, 5,175,273, 6,617,438, 5,594,121, 6,380,368, 5,367,066, 5,587,469, 6,166,197, 5,432,272, 7,495,088, 5,134,066, or 5,596,091. In some embodiments, a nucleobase is described in WO 2020/154344, WO 2020/154343, WO 2020/154342, WO 2020/165077, WO 2020/201406, WO 2020/216637, or WO 2020/252376, and can be utilized in accordance with the present disclosure.

In some embodiments, a nucleobase is a protected base residue as used in oligonucleotide preparation. In some embodiments, a nucleobase is a base residue illustrated in US 2011/0294124, US 2015/0211006, US 2015/0197540, WO 2015/107425, WO 2017/192679, WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the base residues of each of which are independently incorporated herein by reference.

### Base Sequences

In certain embodiments, a base sequence of an oligonucleotide is at least about 50%, about 60%, about 70%, about 75%, about 80%, about 85%, about 90%, about 91%, about 92%, about 93%, about 94%, about 95%, about 96%, about 97%, about 98%, or about 99%, or 100% complementary or identical to a target nucleic acid sequence (e.g., a base sequence of a transcript, RNA, mRNA, etc.)

Base sequences of provided oligonucleotides, as appreciated by those skilled in the art, typically have sufficient length and complementarity to their targets, e.g., RNA transcripts (e.g., pre-mRNA, mature mRNA, etc.) to bind their targets.

Certain sequences are provided, e.g., in Table Al, A2, A3, and A4 as examples. In some embodiments, a base sequence has about 70%, about 75%, about 80%, about 85%, about 90%, about 91%, about 92%, about 93%, about 94%, about 95%, about 96%, about 97%, about 98%, or about 99%, or 100% identity with a base sequence of an oligonucleotide disclosed in a Table, wherein each T can be independently substituted with U and vice versa. In some embodiments, a base sequence has about 85% or more identity with a base sequence of an oligonucleotide disclosed in a Table, wherein each T can be independently substituted with U and vice versa. In some embodiments, a base sequence has about 90% or more identity with a base sequence of an oligonucleotide disclosed in a Table, wherein each T can be independently substituted with U and vice versa. In some embodiments, a base sequence has about 95% or more identity with a base sequence of an oligonucleotide disclosed in a Table, wherein each T can be independently substituted with U and vice versa. In some embodiments, a base sequence has 100% identity with a base sequence of an oligonucleotide disclosed in a Table, wherein each T can be independently substituted with U and vice versa. In some embodiments, a base sequence comprises a continuous span of 15 or more bases of an oligonucleotide disclosed in a Table, wherein each T can be independently substituted with U and vice versa. In some embodiments, a base sequence comprises a continuous span of 16 or more bases of an oligonucleotide disclosed in a Table, wherein each T can be independently substituted with U and vice versa. In some embodiments, a base sequence comprises a continuous span of 17 or more bases of an oligonucleotide disclosed in a Table, wherein each T can be independently substituted with U and vice versa. In some embodiments, a base sequence comprises a continuous span of 18 or more bases of an oligonucleotide disclosed in a Table, wherein each T can be independently substituted with U and vice versa. In some embodiments, a base sequence comprises a continuous span of 19 or more bases of an oligonucleotide disclosed in a Table, wherein each T can be independently substituted with U and vice versa. In some embodiments, a base sequence comprises a continuous span of 20 or more bases of an oligonucleotide disclosed in a Table, wherein each T can be independently substituted with U and vice versa.

In some embodiments, a base sequence of an oligonucleotide comprises 1-5, e.g., 1, 2, or 3 mismatches when align with its target. In some embodiments, one or more or all mismatches are close to or at the 5′-end and/or the 3′-end. As appreciated by those skilled in the art, in some embodiments, sequences of oligonucleotides need not be 100% complementary to their targets for the oligonucleotides to perform their functions. In some embodiments, homology, sequence identity or complementarity is about 60%-100%, e.g., about or at least 60%, 70%, 75%, 80%, 85%, 90%, 91%, 92%, 93%, 94%, 95%, 96%, 97%, 98%, or 99%, or 100%. In some embodiments, a provided oligonucleotide has about 75%-100% (e.g., about or at least 80%, 85%, 90%, 91%, 92%, 93%, 94%, 95%, 96%, 97%, 98%, or 99%, or 100%) sequence complementarity to a target region (e.g., a target sequence) within its target nucleic acid. In some embodiments, the percentage is about 80% or more. In some embodiments, the percentage is about 85% or more. In some embodiments, the percentage is about 90% or more. In some embodiments, the percentage is about 95% or more. Typically when determining complementarity, A and T (or U) are complementary nucleobases and C and G are complementary nucleobases for sequences formed by A, T, C, G and/or U.

### Lengths

As appreciated by those skilled in the art, oligonucleotides can be of various lengths to provide desired properties and/or activities for various uses. Many technologies for assessing, selecting and/or optimizing oligonucleotide length are available in the art and can be utilized in accordance with the present disclosure. As demonstrated herein, in many embodiments, provided oligonucleotides are of suitable lengths to hybridize with their targets and reduce levels of their targets and/or an encoded product thereof. In some embodiments, an oligonucleotide is long enough to recognize a target nucleic acid (e.g., a mRNA). In some embodiments, an oligonucleotide is sufficiently long to distinguish between a target nucleic acid and other nucleic acids to reduce off-target effects. In some embodiments, an oligonucleotide is sufficiently short to reduce complexity of manufacture or production and to reduce cost of products.

In some embodiments, the base sequence of an oligonucleotide is about 10-500 nucleobases in length. In some embodiments, a base sequence is about 10-500 nucleobases in length. In some embodiments, a base sequence is about 10-50 nucleobases in length. In some embodiments, a base sequence is about 15-50 nucleobases in length. In some embodiments, a base sequence is from about 15 to about 30 nucleobases in length. In some embodiments, a base sequence is from about 10 to about 25 nucleobases in length. In some embodiments, a base sequence is from about 15 to about 22 nucleobases in length. In some embodiments, a base sequence is about 10, 11, 12, 13, 14, 15, 16, 17, 18, 19, 20, 21, 22, 23, 24, or 25 nucleobases in length. In some embodiments, a base sequence is at least 12 nucleobases in length. In some embodiments, a base sequence is at least 13 nucleobases in length. In some embodiments, a base sequence is at least 14 nucleobases in length. In some embodiments, a base sequence is at least 15 nucleobases in length. In some embodiments, a base sequence is at least 16 nucleobases in length. In some embodiments, a base sequence is at least 17 nucleobases in length. In some embodiments, a base sequence is at least 18 nucleobases in length. In some embodiments, a base sequence is at least 19 nucleobases in length. In some embodiments, a base sequence is at least 20 nucleobases in length. In some embodiments, a base sequence is at least 21 nucleobases in length. In some embodiments, a base sequence is at least 22 nucleobases in length. In some embodiments, a base sequence is at least 23 nucleobases in length. In some embodiments, a base sequence is at least 24 nucleobases in length. In some embodiments, a base sequence is at least 25 nucleobases in length. In some embodiments, a base sequence is 15 nucleobases in length. In some embodiments, a base sequence is 16 nucleobases in length. In some embodiments, a base sequence is 17 nucleobases in length. In some embodiments, a base sequence is 18 nucleobases in length. In some embodiments, a base sequence is 19 nucleobases in length. In some embodiments, a base sequence is 20 nucleobases in length. In some embodiments, a base sequence is 21 nucleobases in length. In some embodiments, a base sequence is 22 nucleobases in length. In some embodiments, a base sequence is 23 nucleobases in length. In some embodiments, a base sequence is 24 nucleobases in length. In some embodiments, a base sequence is 25 nucleobases in length. In some other embodiments, a base sequence is at least 30 nucleobases in length. In some other embodiments, a base sequence is a duplex of complementary strands of at least 18 nucleobases in length. In some other embodiments, a base sequence is a duplex of complementary strands of at least 21 nucleobases in length. In some embodiments, each nucleobase independently comprises an optionally substituted monocyclic, bicyclic or polycyclic ring wherein at least one ring atom is nitrogen. In some embodiments, each nucleobase counted in length independently comprises an optionally substituted monocyclic, bicyclic or polycyclic ring wherein at least one ring atom is nitrogen. In some embodiments, each nucleobase is independently optionally substituted adenine, cytosine, guanosine, thymine, or uracil, or an optionally substituted tautomer of adenine, cytosine, guanosine, thymine, or uracil. In some embodiments, each nucleobase counted in length is independently optionally substituted adenine, cytosine, guanosine, thymine, or uracil, or an optionally substituted tautomer of adenine, cytosine, guanosine, thymine, or uracil.

### Stereochemistry and Patterns of Backbone Chiral Centers

In contrast to natural phosphate linkages, linkage phosphorus of chiral modified internucleotidic linkages, e.g., phosphorothioate internucleotidic linkages, are chiral. Among other things, the present disclosure provides technologies (e.g., oligonucleotides, compositions, methods, etc.) comprising control of stereochemistry of chiral linkage phosphorus in chiral internucleotidic linkages. In some embodiments, control of stereochemistry can provide improved properties and/or activities, including desired stability, reduced toxicity, improved reduction of target nucleic acids, etc. In some embodiments, the present disclosure provides useful patterns of backbone chiral centers for oligonucleotides and/or regions thereof, which pattern is a combination of stereochemistry of each chiral linkage phosphorus (Rp or Sp) of chiral linkage phosphorus, indication of each achiral linkage phosphorus (Op, if any), etc. from 5′ to 3′. In some embodiments, patterns of backbone chiral centers can control cleavage patterns of target nucleic acids when they are contacted with provided oligonucleotides or compositions thereof in a cleavage system (e.g., in vitro assay, cells, tissues, organs, organisms, subjects, etc.). In some embodiments, patterns of backbone chiral centers improve cleavage efficiency and/or selectivity of target nucleic acids when they are contacted with provided oligonucleotides or compositions thereof in a cleavage system. In some embodiments, patterns of backbone chiral centers improve activities and/or properties, e.g., editing, splicing modulation, cleavage, inhibition, stability, delivery, toxicity, clearance, etc.

In some embodiments, a pattern of backbone chiral centers of an oligonucleotide or a region thereof comprises or is (Np)n(Op)m, wherein Np is Rp or Sp, Op represents a linkage phosphorus being achiral (e.g., as for the linkage phosphorus of natural phosphate linkages), and each of n and m is independently 1-50. In some embodiments, a pattern of backbone chiral centers of an oligonucleotide or a region thereof comprises or is (Rp)n(Sp)m, wherein each of n and m is independently as defined and described in the present disclosure. In some embodiments, a pattern of backbone chiral centers of an oligonucleotide or a region thereof comprises or is Rp(Sp)m, wherein each of n and m is independently as defined and described in the present disclosure. In some embodiments, a pattern of backbone chiral centers of an oligonucleotide or a region thereof comprises or is (Sp)n(Op)m, wherein each variable is independently as defined and described in the present disclosure. In some embodiments, a pattern of backbone chiral centers of an oligonucleotide or a region thereof comprises or is (Rp)n(Op)m, wherein each variable is independently as defined and described in the present disclosure. In some embodiments, n is 1. In some embodiments, a pattern of backbone chiral centers of an oligonucleotide or a region thereof comprises or is (Sp)(Op)m, wherein m is 1, 2, 3, 4, 5, 6, 7, 8, 9, or 10. In some embodiments, a pattern of backbone chiral centers of an oligonucleotide or a region thereof comprises or is (Rp)(Op)m, wherein m is 1, 2, 3, 4, 5, 6, 7, 8, 9, or 10. In some embodiments, as described in the present disclosure, m is 2; in some embodiments, m is 3; in some embodiments, m is 4; in some embodiments, m is 5; in some embodiments, m is 6.

In some embodiments, a pattern of backbone chiral centers of an oligonucleotide or a region thereof comprises or is (Op)m(Np)n, wherein Np is Rp or Sp, Op represents a linkage phosphorus being achiral (e.g., as for the linkage phosphorus of natural phosphate linkages), and each of n and m is independently as defined and described in the present disclosure. In some embodiments, a pattern of backbone chiral centers of an oligonucleotide or a region thereof comprises or is (Op)m(Sp)n, wherein each variable is independently as defined and described in the present disclosure. In some embodiments, a pattern of backbone chiral centers of an oligonucleotide or a region thereof comprises or is (Op)m(Rp)n, wherein each variable is independently as defined and described in the present disclosure. In some embodiments, n is 1. In some embodiments, a pattern of backbone chiral centers of an oligonucleotide or a region thereof comprises or is (Op)m(Sp), wherein m is 1, 2, 3, 4, 5, 6, 7, 8, 9, or 10. In some embodiments, a pattern of backbone chiral centers of an oligonucleotide or a region thereof comprises or is (Op)m(Rp), wherein m is 1, 2, 3, 4, 5, 6, 7, 8, 9, or 10. In some embodiments, as described in the present disclosure, m is 2; in some embodiments, m is 3; in some embodiments, m is 4; in some embodiments, m is 5; in some embodiments, m is 6.

In some embodiments, at least one or each Rp is the configuration of a chiral non-negatively charged internucleotidic linkage, e.g., n001. In some embodiments, at least one or each Rp is the configuration of a phosphorothioate internucleotidic linkage.

In some embodiments, a pattern of backbone chiral centers of an oligonucleotide or a region thereof comprises or is any (Np)n(Op)m, wherein Np is Rp or Sp, Op represents a linkage phosphorus being achiral (e.g., as for the linkage phosphorus of natural phosphate linkages), and each of n and m is independently as defined and described in the present disclosure. In some embodiments, a pattern of backbone chiral centers of an oligonucleotide or a region thereof comprises or is (Sp)n(Op)m, wherein each variable is independently as defined and described in the present disclosure. In some embodiments, a pattern of backbone chiral centers of an oligonucleotide or a region thereof comprises or is (Rp)n(Op)m, wherein each variable is independently as defined and described in the present disclosure. In some embodiments, n is 1. In some embodiments, a pattern of backbone chiral centers of an oligonucleotide or a region thereof comprises or is (Sp)(Op)m, wherein m is 1, 2, 3, 4, 5, 6, 7, 8, 9, or 10. In some embodiments, a pattern of backbone chiral centers of an oligonucleotide or a region thereof comprises or is (Rp)(Op)m, wherein m is 1, 2, 3, 4, 5, 6, 7, 8, 9, or 10. In some embodiments, the pattern of backbone chiral centers of a 5′-wing is or comprises (Np)n(Op)m. In some embodiments, the pattern of backbone chiral centers of a 5′-wing is or comprises (Sp)n(Op)m. In some embodiments, the pattern of backbone chiral centers of a 5′-wing is or comprises (Rp)n(Op)m. In some embodiments, the pattern of backbone chiral centers of a 5′-wing is or comprises (Sp)(Op)m. In some embodiments, the pattern of backbone chiral centers of a 5′-wing is or comprises (Rp)(Op)m. In some embodiments, the pattern of backbone chiral centers of a 5′-wing is (Sp)(Op)m. In some embodiments, the pattern of backbone chiral centers of a 5′-wing is (Rp)(Op)m. In some embodiments, the pattern of backbone chiral centers of a 5′-wing is (Sp)(Op)m, wherein Sp is the linkage phosphorus configuration of the first internucleotidic linkage of the oligonucleotide from the 5′-end. In some embodiments, the pattern of backbone chiral centers of a 5′-wing is (Rp)(Op)m, wherein Rp is the linkage phosphorus configuration of the first internucleotidic linkage of the oligonucleotide from the 5′-end. In some embodiments, as described in the present disclosure, m is 2; in some embodiments, m is 3; in some embodiments, m is 4; in some embodiments, m is 5; in some embodiments, m is 6.

In some embodiments, a pattern of backbone chiral centers of an oligonucleotide or a region thereof comprises or is (Op)m(Np)n, wherein Np is Rp or Sp, Op represents a linkage phosphorus being achiral (e.g., as for the linkage phosphorus of natural phosphate linkages), and each of n and m is independently as defined and described in the present disclosure. In some embodiments, a pattern of backbone chiral centers of an oligonucleotide or a region thereof comprises or is (Op)m(Sp)n, wherein each variable is independently as defined and described in the present disclosure. In some embodiments, a pattern of backbone chiral centers of an oligonucleotide or a region thereof comprises or is (Op)m(Rp)n, wherein each variable is independently as defined and described in the present disclosure. In some embodiments, n is 1. In some embodiments, a pattern of backbone chiral centers of an oligonucleotide or a region thereof comprises or is (Op)m(Sp), wherein m is 1, 2, 3, 4, 5, 6, 7, 8, 9, or 10. In some embodiments, a pattern of backbone chiral centers of an oligonucleotide or a region thereof comprises or is (Op)m(Rp), wherein m is 1, 2, 3, 4, 5, 6, 7, 8, 9, or 10. In some embodiments, the pattern of backbone chiral centers of a 3′-wing is or comprises (Op)m(Np)n. In some embodiments, the pattern of backbone chiral centers of a 3′-wing is or comprises (Op)m(Sp)n. In some embodiments, the pattern of backbone chiral centers of a 3′-wing is or comprises (Op)m(Rp)n. In some embodiments, the pattern of backbone chiral centers of a 3′-wing is or comprises (Op)m(Sp). In some embodiments, the pattern of backbone chiral centers of a 3′-wing is or comprises (Op)m(Rp). In some embodiments, the pattern of backbone chiral centers of a 3′-wing is (Op)m(Sp). In some embodiments, the pattern of backbone chiral centers of a 3′-wing is (Op)m(Rp). In some embodiments, the pattern of backbone chiral centers of a 3′-wing is (Op)m(Sp), wherein Sp is the linkage phosphorus configuration of the last internucleotidic linkage of the oligonucleotide from the 5′-end. In some embodiments, the pattern of backbone chiral centers of a 3′-wing is (Op)m(Rp), wherein Rp is the linkage phosphorus configuration of the last internucleotidic linkage of the oligonucleotide from the 5′-end. In some embodiments, as described in the present disclosure, m is 2; in some embodiments, m is 3; in some embodiments, m is 4; in some embodiments, m is 5; in some embodiments, m is 6.

In some embodiments, a pattern of backbone chiral centers of an oligonucleotide or a region thereof (e.g., a core) comprises or is (Sp)m(Rp/Op)n or (Rp/Op)n(Sp)m, wherein each variable is independently as described in the present disclosure. In some embodiments, a pattern of backbone chiral centers of an oligonucleotide or a region thereof (e.g., a core) comprises or is (Sp)m(Rp)n or (Rp)n(Sp)m, wherein each variable is independently as described in the present disclosure. In some embodiments, a pattern of backbone chiral centers of an oligonucleotide or a region thereof (e.g., a core) comprises or is (Sp)m(Op)n or (Op)n(Sp)m, wherein each variable is independently as described in the present disclosure. In some embodiments, a pattern of backbone chiral centers of an oligonucleotide or a region thereof (e.g., a core) comprises or is (Np)t[(Rp/Op)n(Sp)m]y or [(Rp/Op)n(Sp)m]y(Np)t, wherein y is 1-50, and each other variable is independently as described in the present disclosure. In some embodiments, a pattern of backbone chiral centers of an oligonucleotide or a region thereof (e.g., a core) comprises or is (Np)t[(Rp)n(Sp)m]y or [(Rp)n(Sp)m]y(Np)t, wherein each variable is independently as described in the present disclosure. In some embodiments, a pattern of backbone chiral centers of an oligonucleotide or a region thereof (e.g., a core) comprises or is [(Rp/Op)n(Sp)m]y(Rp)k, [(Rp/Op)n(Sp)m]y, (Sp)t[(Rp/Op)n(Sp)m]y, (Sp)t[(Rp/Op)n(Sp)m]y(Rp)k, wherein k is 1-50, and each other variable is independently as described in the present disclosure. In some embodiments, a pattern of backbone chiral centers of an oligonucleotide or a region thereof (e.g., a core) comprises or is [(Op)n(Sp)m]y(Rp)k, [(Op)n(Sp)m]y, (Sp)t[(Pp)n(Sp)m]y, (Sp)t[(Pp)n(Sp)m]y(Rp)k, wherein each variable is independently as described in the present disclosure. In some embodiments, a pattern of backbone chiral centers of an oligonucleotide or a region thereof (e.g., a core) comprises or is [(Rp)n(Sp)m]y(Rp)k, [(Rp)n(Sp)m]y, (Sp)t[(Rp)n(Sp)m]y, (Sp)t[(Rp)n(Sp)m]y(Rp)k, wherein each variable is independently as described in the present disclosure. In some embodiments, an oligonucleotide comprises a core region. In some embodiments, an oligonucleotide comprises a core region, wherein each sugar in the core region does not contain a 2′-OR¹, wherein R¹ is as described in the present disclosure. In some embodiments, an oligonucleotide comprises a core region, wherein each sugar in the core region is independently a natural DNA sugar. In some embodiments, the pattern of backbone chiral centers of the core comprises or is (Rp)(Sp)m. In some embodiments, the pattern of backbone chiral centers of the core comprises or is (Op)(Sp)m. In some embodiments, the pattern of backbone chiral centers of the core comprises or is (Np)t[(Rp/Op)n(Sp)m]y or [(Rp/Op)n(Sp)m]y(Np)t. In some embodiments, the pattern of backbone chiral centers of the core comprises or is (Np)t[(Rp/Op)n(Sp)m]y or [(Rp/Op)n(Sp)m]y(Np)t. In some embodiments, the pattern of backbone chiral centers of the core comprises or is (Np)t[(Rp)n(Sp)m]y or [(Rp)n(Sp)m]y(Np)t. In some embodiments, the pattern of backbone chiral centers of a core comprises or is [(Rp/Op)n(Sp)m]y(Rp)k, [(Rp/Op)n(Sp)m]y, (Sp)t[(Rp/Op)n(Sp)m]y, (Sp)t[(Rp/Op)n(Sp)m]y(Rp)k. In some embodiments, a pattern of backbone chiral centers of a core comprises or is [(Op)n(Sp)m]y(Rp)k, [(Op)n(Sp)m]y, (Sp)t[(Pp)n(Sp)m]y, (Sp)t[(Pp)n(Sp)m]y(Rp)k. In some embodiments, a pattern of backbone chiral centers of a core comprises or is [(Rp)n(Sp)m]y(Rp)k, [(Rp)n(Sp)m]y, (Sp)t[(Rp)n(Sp)m]y, or (Sp)t[(Rp)n(Sp)m]y(Rp)k. In some embodiments, a pattern of backbone chiral centers of a core comprises [(Rp)n(Sp)m]y(Rp)k. In some embodiments, a pattern of backbone chiral centers of a core comprises [(Rp)n(Sp)m]y(Rp). In some embodiments, a pattern of backbone chiral centers of a core comprises [(Rp)n(Sp)m]y. In some embodiments, a pattern of backbone chiral centers of a core comprises (Sp)t[(Rp)n(Sp)m]y. In some embodiments, a pattern of backbone chiral centers of a core comprises (Sp)t[(Rp)n(Sp)m]y(Rp)k. In some embodiments, a pattern of backbone chiral centers of a core comprises (Sp)t[(Rp)n(Sp)m]y(Rp). In some embodiments, a pattern of backbone chiral centers of a core is [(Rp)n(Sp)m]y(Rp)k. In some embodiments, a pattern of backbone chiral centers of a core is [(Rp)n(Sp)m]y(Rp). In some embodiments, a pattern of backbone chiral centers of a core is [(Rp)n(Sp)m]y. In some embodiments, a pattern of backbone chiral centers of a core is (Sp)t[(Rp)n(Sp)m]y. In some embodiments, a pattern of backbone chiral centers of a core is (Sp)t[(Rp)n(Sp)m]y(Rp)k. In some embodiments, a pattern of backbone chiral centers of a core is (Sp)t[(Rp)n(Sp)m]y(Rp). In some embodiments, each n is 1. In some embodiments, each t is 1. In some embodiments, t is 2, 3, 4, 5, 6, 7, 8, 9, or 10. In some embodiments, each oft and n is 1. In some embodiments, each m is 2 or more. In some embodiments, k is 1. In some embodiments, k is 2-10.

In some embodiments, a pattern of backbone chiral centers comprises or is (Sp)m(Rp)n, (Rp)n(Sp)m, (Np)t(Rp)n(Sp)m, (Sp)t(Rp)n(Sp)m, (Np)t[(Rp)n(Sp)m]2, (Sp)t[(Rp)n(Sp)m]2, (Np)t(Op)n(Sp)m, (Sp)t(Op)n(Sp)m, (Np)t[(Pp)n(Sp)m]2, or (Sp)t[(Pp)n(Sp)m]2. In some embodiments, a pattern is (Np)t(Op/Rp)n(Sp)m(Op/Rp)n(Sp)m. In some embodiments, a pattern is (Np)t(Op/Rp)n(Sp)₁-5(Op/Rp)n(Sp)m. In some embodiments, a pattern is (Np)t(Op/Rp)n(Sp)—S(Op/Rp)n(Sp)m. In some embodiments, a pattern is (Np)t(Op/Rp)n(Sp)₂(Op/Rp)n(Sp)m. In some embodiments, a pattern is (Np)t(Op/Rp)n(Sp)₃(Op/Rp)n(Sp)m In some embodiments, a pattern is (Np)t(Op/Rp)n(Sp)t(Op/Rp)n(Sp)m. In some embodiments, a pattern is (Np)t(Op/Rp)n(Sp)₅(Op/Rp)n(Sp)m.

In some embodiments, Np is Sp. In some embodiments, (Op/Rp) is Op. In some embodiments, (Op/Rp) is Rp. In some embodiments, Np is Sp and (Op/Rp) is Rp. In some embodiments, Np is Sp and (Op/Rp) is Op. In some embodiments, Np is Sp and at least one (Op/Rp) is Rp, and at least one (Op/Rp) is Op. In some embodiments, a pattern of backbone chiral centers comprises or is (Rp)n(Sp)m, (Np)t(Rp)n(Sp)m, or (Sp)t(Rp)n(Sp)m, wherein m >2. In some embodiments, a pattern of backbone chiral centers comprises or is (Rp)n(Sp)m, (Np)t(Rp)n(Sp)m, or (Sp)t(Rp)n(Sp)m, wherein n is 1, at least one t >1, and at least one m >2.

In some embodiments, oligonucleotides comprising core regions whose patterns of backbone chiral centers starting with Rp can provide high activities and/or improved properties. In some embodiments, oligonucleotides comprising core regions whose patterns of backbone chiral centers ending with Rp can provide high activities and/or improved properties. In some embodiments, oligonucleotides comprising core regions whose patterns of backbone chiral centers starting with Rp provide high activities (e.g., target cleavage) without significantly impacting its properties, e.g., stability. In some embodiments, oligonucleotides comprising core regions whose patterns of backbone chiral centers ending with Rp provide high activities (e.g., target cleavage) without significantly impacting its properties, e.g., stability. In some embodiments, patterns of backbone chiral centers start with Rp and end with Sp. In some embodiments, patterns of backbone chiral centers start with Rp and end with Rp. In some embodiments, patterns of backbone chiral centers start with Sp and end with Rp. Typically, for patterns of backbone chiral centers internucleotidic linkages connecting core nucleosides and wing nucleosides are included in the patterns of the core regions. In many embodiments as described in the present disclosure (e.g., various oligonucleotides in Table 1), the wing sugar connected by such a connecting internucleotidic linkage has a different structure than the core sugar connected by the same connecting internucleotidic linkage (e.g., in some embodiments, the wing sugar comprises a 2′-modification while the core sugar does not contain the same 2′-modification or have two —H at the 2′ position). In some embodiments, the wing sugar comprises a sugar modification that the core sugar does not contain. In some embodiments, the wing sugar is a modified sugar while the core sugar is a natural DNA sugar. In some embodiments, the wing sugar comprises a sugar modification at the 2′ position (less than two —H at the 2′ position), and the core sugar has no modification at the 2′-position (two —H at the 2′ position).

In some embodiments, as demonstrated herein, an additional Rp internucleotidic linkage links a sugar containing no 2′-substituent (e.g., a core sugar) and a sugar comprising a 2′-modification (e.g., 2′-OR′, wherein R′ is optionally substituted C₁₋₆ aliphatic (e.g., 2′-OMe, 2′-MOE, etc.), which can be a wing sugar). In some embodiments, an internucleotidic linkage linking a sugar containing no 2′-substituent to the 5′-end (e.g., to the 3′-carbon of the sugar) and a sugar comprising a 2′-modification to the 3′-end (e.g., to the 5′-carbon of the sugar) is a Rp internucleotidic linkage. In some embodiments, an internucleotidic linkage linking a sugar containing no 2′-substituent to the 3′-end (e.g., to the 5′-carbon of the sugar) and a sugar comprising a 2′-modification to the 5′-end (e.g., to the 3′-carbon of the sugar) is a Rp internucleotidic linkage. In some embodiments, each internucleotidic linkage linking a sugar containing no 2′-substituent and a sugar comprising a 2′-modification is independently a Rp internucleotidic linkage. In some embodiments, a Rp internucleotidic linkage is a Rp phosphorothioate internucleotidic linkage.

In some embodiments, a pattern of backbone chiral centers of an oligonucleotide or a region thereof (e.g., a core) comprises or is (Op)[(Rp/Op)n(Sp)m]y(Rp)k(Op), (Op)[(Rp/Op)n(Sp)m]y(Op), (Op)(Sp)t[(Rp/Op)n(Sp)m]y(Op), or (Op)(Sp)t[(Rp/Op)n(Sp)m]y(Rp)k(Op), wherein k is 1-50, and each other variable is independently as described in the present disclosure. In some embodiments, a pattern of backbone chiral centers of an oligonucleotide comprises or is (Op)[(Rp/Op)n(Sp)m]y(Rp)k(Op), (Op)[(Rp/Op)n(Sp)m]y(Op), (Op)(Sp)t[(Rp/Op)n(Sp)m]y(Op), or (Op)(Sp)t[(Rp/Op)n(Sp)m]y(Rp)k(Op), wherein each of f, g, h and j is independently 1-50, and each other variable is independently as described in the present disclosure, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In some embodiments, a pattern of backbone chiral centers is or comprises (Op)[(Rp/Op)n(Sp)m]y(Rp)k(Op). In some embodiments, a pattern of backbone chiral centers is or comprises (Op)[(Rp/Op)n(Sp)m]y(Rp)(Op). In some embodiments, a pattern of backbone chiral centers is or comprises (Op)[(Rp/Op)n(Sp)m]y(Op). In some embodiments, a pattern of backbone chiral centers is or comprises (Op)(Sp)t[(Rp/Op)n(Sp)m]y(Op). In some embodiments, a pattern of backbone chiral centers is or comprises (Op)(Sp)t[(Rp/Op)n(Sp)m]y(Rp)k(Op). In some embodiments, a pattern of backbone chiral centers is or comprises (Op)(Sp)t[(Rp/Op)n(Sp)m]y(Rp)(Op). In some embodiments, a pattern of backbone chiral centers is or comprises (Op)[(Rp)n(Sp)m]y(Rp)k(Op). In some embodiments, a pattern of backbone chiral centers is or comprises (Op)[(Rp)n(Sp)m]y(Rp)(Op). In some embodiments, a pattern of backbone chiral centers is or comprises (Op)[(Rp)n(Sp)m]y(Op). In some embodiments, a pattern of backbone chiral centers is or comprises (Op)(Sp)t[(Rp)n(Sp)m]y(Op). In some embodiments, a pattern of backbone chiral centers is or comprises (Op)(Sp)t[(Rp)n(Sp)m]y(Rp)k(Op). In some embodiments, a pattern of backbone chiral centers is or comprises (Op)(Sp)t[(Rp)n(Sp)m]y(Rp)(Op). In some embodiments, each n is 1. In some embodiments, k is 1. In some embodiments, k is 2-10.

In some embodiments, a pattern of backbone chiral centers of an oligonucleotide or a region thereof (e.g., a core) comprises or is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, wherein each of f, g, h and j is independently 1-50, and each other variable is independently as described in the present disclosure. In some embodiments, a pattern of backbone chiral centers of an oligonucleotide comprises or is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In some embodiments, a pattern of backbone chiral centers of an oligonucleotide is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In some embodiments, a pattern of backbone chiral centers is or comprises (Np)f(Op)g[(Rp/Op)n(Sp)m]y(Rp)k(Op)h(Np)j. In some embodiments, a pattern of backbone chiral centers is or comprises (Np)f(Op)g[(Rp/Op)n(Sp)m]y(Rp)(Op)h(Np)j. In some embodiments, a pattern of backbone chiral centers is or comprises (Np)f(Op)g[(Rp/Op)n(Sp)m]y(Op)h(Np)j. In some embodiments, a pattern of backbone chiral centers is or comprises (Np)f(Op)g(Sp)t[(Rp/Op)n(Sp)m]y(Op)h(Np)j. In some embodiments, a pattern of backbone chiral centers is or comprises (Np)f(Op)g(Sp)t[(Rp/Op)n(Sp)m]y(Rp)k(Op)h(Np)j. In some embodiments, a pattern of backbone chiral centers is or comprises (Np)f(Op)g(Sp)t[(Rp/Op)n(Sp)m]y(Rp)(Op)h(Np)j. In some embodiments, a pattern of backbone chiral centers is or comprises (Np)f(Op)g[(Rp)n(Sp)m]y(Rp)k(Op)h(Np)j. In some embodiments, a pattern of backbone chiral centers is or comprises (Np)f(Op)g[(Rp)n(Sp)m]y(Rp)(Op)h(Np)j. In some embodiments, a pattern of backbone chiral centers is or comprises (Np)f(Op)g[(Rp)n(Sp)m]y(Op)h(Np)j. In some embodiments, a pattern of backbone chiral centers is or comprises (Np)f(Op)g(Sp)t[(Rp)n(Sp)m]y(Op)h(Np)j. In some embodiments, a pattern of backbone chiral centers is or comprises (Np)f(Op)g(Sp)t[(Rp)n(Sp)m]y(Rp)k(Op)h(Np)j. In some embodiments, a pattern of backbone chiral centers is or comprises (Np)f(Op)g(Sp)t[(Rp)n(Sp)m]y(Rp)(Op)h(Np)j. In some embodiments, at least one Np is Sp. In some embodiments, at least one Np is Rp. In some embodiments, the 5′ most Np is Sp. In some embodiments, the 3′ most Np is Sp. In some embodiments, each Np is Sp. In some embodiments, (Np)f(Op)g[(Rp/Op)n(Sp)m]y(Rp)k(Op)h(Np)j is (Sp)(Op)g[(Rp)n(Sp)m]y(Rp)k(Op)h(Sp). In some embodiments, (Np)f(Op)g[(Rp/Op)n(Sp)m]y(Rp)k(Op)h(Np)j is (Sp)(Op)g[(Rp)n(Sp)m]y(Rp)(Op)h(Sp). In some embodiments, a pattern of backbone chiral center of an oligonucleotide is or comprises (Sp)(Op)g[(Rp)n(Sp)m]y(Rp)(Op)h(Sp). In some embodiments, a pattern of backbone chiral center of an oligonucleotide is (Sp)(Op)g[(Rp)n(Sp)m]y(Rp)(Op)h(Sp). In some embodiments, (Np)f(Op)g[(Rp/Op)n(Sp)m]y(Op)h(Np)j is (Sp)(Op)g[(Rp)n(Sp)m]y(Op)h(Sp). In some embodiments, a pattern of backbone chiral center of an oligonucleotide is or comprises (Sp)(Op)g[(Rp)n(Sp)m]y(Op)h(Sp). In some embodiments, a pattern of backbone chiral center of an oligonucleotide is (Sp)(Op)g[(Rp)n(Sp)m]y(Op)h(Sp). In some embodiments, (Np)f(Op)g(Sp)t[(Rp/Op)n(Sp)m]y(Op)h(Np)j is (Sp)(Op)g(Sp)t[(Rp)n(Sp)m]y(Op)h(Sp). In some embodiments, a pattern of backbone chiral center of an oligonucleotide is or comprises (Sp)(Op)g(Sp)t[(Rp)n(Sp)m]y(Op)h(Sp). In some embodiments, a pattern of backbone chiral center of an oligonucleotide is (Sp)(Op)g(Sp)t[(Rp)n(Sp)m]y(Op)h(Sp). In some embodiments, (Np)f(Op)g(Sp)t[(Rp/Op)n(Sp)m]y(Rp)k(Op)h(Np)j is (Sp)(Op)g(Sp)t[(Rp)n(Sp)m]y(Rp)k(Op)h(Sp). In some embodiments, (Np)f(Op)g(Sp)t[(Rp/Op)n(Sp)m]y(Rp)k(Op)h(Np)j is (Sp)(Op)g(Sp)t[(Rp)n(Sp)m]y(Rp)(Op)h(Sp). In some embodiments, a pattern of backbone chiral center of an oligonucleotide is or comprises (Sp)(Op)g(Sp)t[(Rp)n(Sp)m]y(Rp)(Op)h(Sp). In some embodiments, a pattern of backbone chiral center of an oligonucleotide is (Sp)(Op)g(Sp)t[(Rp)n(Sp)m]y(Rp)(Op)h(Sp). In some embodiments, each n is 1. In some embodiments, f is 1. In some embodiments, g is 1. In some embodiments, g is greater than 1. In some embodiments, g is 2. In some embodiments, g is 3. In some embodiments, g is 4. In some embodiments, g is 5. In some embodiments, g is 6. In some embodiments, g is 7. In some embodiments, g is 8. In some embodiments, g is 9. In some embodiments, g is 10. In some embodiments, h is 1. In some embodiments, h is greater than 1. In some embodiments, h is 2. In some embodiments, h is 3. In some embodiments, h is 4. In some embodiments, h is 5. In some embodiments, h is 6. In some embodiments, h is 7. In some embodiments, h is 8. In some embodiments, h is 9. In some embodiments, h is 10. In some embodiments, j is 1. In some embodiments, k is 1. In some embodiments, k is 2-10.

In some embodiments, a pattern of backbone chiral centers of an oligonucleotide or a region thereof (e.g., a core) comprises or is [(Rp/Op)n(Sp)m]y, (Sp)t[(Rp/Op)n(Sp)m]y, (Sp)t[(Rp/Op)n(Sp)m]yRp, [(Rp/Op)n(Sp)m]y(Rp)k, (Sp)t[(Rp/Op)n(Sp)m]y(Rp)k, (Sp)t[(Rp/Op)n(Sp)m]y(Rp)k(Op)h, (Sp)t[(Rp/Op)n(Sp)m]y(Rp)k(Op)h(Np)j, wherein each variable is independently as described in the present disclosure.

In some embodiments, in a provided pattern of backbone chiral centers, at least one (Rp/Op) is Rp. In some embodiments, at least one (Rp/Op) is Op. In some embodiments, each (Rp/Op) is Rp. In some embodiments, each (Rp/Op) is Op. In some embodiments, at least one of [(Rp)n(Sp)m]y or [(Rp/Op)n(Sp)m]y of a pattern is RpSp. In some embodiments, at least one of [(Rp)n(Sp)m]y or [(Rp/Op)n(Sp)m]y of a pattern is or comprises RpSpSp. In some embodiments, at least one of [(Rp)n(Sp)m]y or [(Rp/Op)n(Sp)m]y in a pattern is RpSp, and at least one of [(Rp)n(Sp)m]y or [(Rp/Op)n(Sp)m]y in a pattern is or comprises RpSpSp. For example, in some embodiments, [(Rp)n(Sp)m]y in a pattern is (RpSp)[(Rp)n(Sp)m]_{(y-1)}; in some embodiments, [(Rp)n(Sp)m]y in a pattern is (RpSp)[RpSpSp(Sp)₍ₘ₋₂₎][(Rp)n(Sp)m]_{(y-2)}. In some embodiments, (Sp)t[(Rp)n(Sp)m]y(Rp) is (Sp)t(RpSp)[(Rp)n(Sp)m]_{(y-1)}(Rp). In some embodiments, (Sp)t[(Rp)n(Sp)m]y(Rp) is (Sp)t(RpSp)[RpSpSp(Sp)(ₘ₋₂)][(Rp)n(Sp)m]_{(y-2)}(Rp). In some embodiments, each [(Rp/Op)n(Sp)m] is independently [Rp(Sp)m]. In some embodiments, the first Sp of (Sp)t represents linkage phosphorus stereochemistry of the first internucleotidic linkage of an oligonucleotide from 5′ to 3′. In some embodiments, the first Sp of (Sp)t represents linkage phosphorus stereochemistry of the first internucleotidic linkage of a region from 5′ to 3′, e.g., a core. In some embodiments, the last Np of (Np)j represents linkage phosphorus stereochemistry of the last internucleotidic linkage of the oligonucleotide from 5′ to 3′. In some embodiments, the last Np is Sp.

In some embodiments, a pattern of backbone chiral centers of a core comprises or is [(Rp(Sp)m]y, (Np)t[Rp(Sp)m]y, or (Sp)t[Rp(Sp)m]y. In some embodiments, m is 2 or more. In some embodiments, m is 2, 3, 4, 5, 6, 7, 8, 9, 10. In some embodiments, t is one or more. In some embodiments, t is 1, 2, 3, 4, 5, 6, 7, 8, 9 or 10. In some embodiments, there are about or at least about 1-20, e.g., about or at least about 1, 2, 3, 4, 5, 6, 7, 8, 9, 10, 11, 12, 13, 14, 15, 16, 17, 18, 19, or 20 internucleotidic linkages, each of which is independently bonded to one or more core sugars, to the 5′ side of a core internucleotidic linkage whose configuration is the Rp of [(Rp(Sp)m]y, (Np)t[Rp(Sp)m]y, or (Sp)t[Rp(Sp)m]y. In some embodiments, it is about or at least about 1. In some embodiments, it is about or at least about 2. In some embodiments, it is about or at least about 3. In some embodiments, it is about or at least about 4. In some embodiments, it is about or at least about 5. In some embodiments, it is about or at least about 6. In some embodiments, it is about or at least about 7. In some embodiments, it is about or at least about 8. In some embodiments, it is about or at least about 9. In some embodiments, it is about or at least about 10. In some embodiments, the internucleotidic linkage whose configuration is the Rp of [(Rp(Sp)m]y, (Np)t[Rp(Sp)m]y, or (Sp)t[Rp(Sp)m]y is the 5^{th}, 6^{th}, 7^{th}, 8^{th}, 9^{th}, 10^{th}, 11^{th} or 12^{th} internucleotidic linkage that is bonded to at least one core sugar. In some embodiments, each Sp of [(Rp(Sp)m]y, (Np)t[Rp(Sp)m]y, or (Sp)t[Rp(Sp)m]y is independently the configuration of an internucleotidic linkage which is bonded to at least one core sugar. In some embodiments, a sugar comprising nitrogen is at position +1, +2, +3, +4, +5, +6, +7, +8, −1, −2, −3, −4, −5, −6, −7, or −8 relative to the Rp internucleotidic linkage (5′- . . . N₊₄ N₊₃ N₊₂ N₊₁ N₋₁ N₋₂ N₋₃ N₋₄. . . −3′, wherein Rp is the configuration of the internucleotidic linkage connecting N₊₁ and N₋₁). In some embodiments, a position is +1. In some embodiments, a position is +2. In some embodiments, a position is +3. In some embodiments, a position is +4. In some embodiments, a position is +5. In some embodiments, a position is +6. In some embodiments, a position is +7. In some embodiments, a position is +8. In some embodiments, a position is −1. In some embodiments, a position is −2. In some embodiments, a position is −3. In some embodiments, a position is −4. In some embodiments, a position is −5. In some embodiments, a position is −6. In some embodiments, a position is −7. In some embodiments, a position is −8. In some embodiments, a sugar comprising nitrogen is

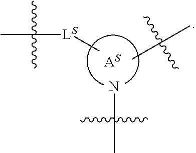

In some embodiments, a sugar comprising nitrogen is sm01. In some embodiments, it forms sm01n001

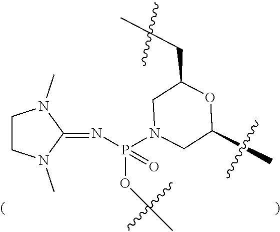

with an internucleotidic linkage

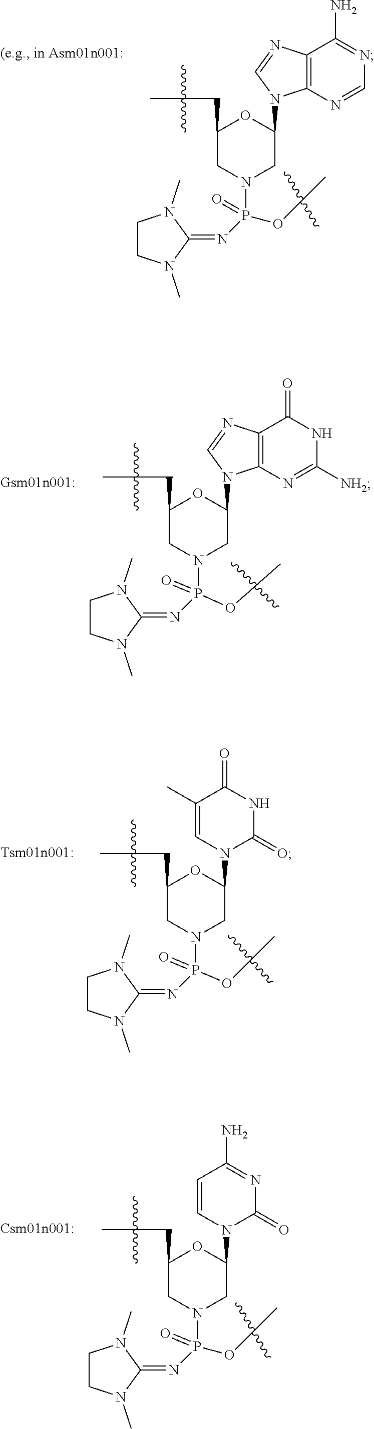 [SMILES: *C[C@@H]1CN(P(=O)(N=C2N(C)CCN2C)O*)C[C@H](n2cnc3c(N)ncnc32)O1 ; *C[C@@H]1CN(P(=O)(N=C2N(C)CCN2C)O*)C[C@H](n2cnc3c(=O)[nH]c(N)nc32)O1 ; *C[C@@H]1CN(P(=O)(N=C2N(C)CCN2C)O*)C[C@H](n2cc(C)c(=O)[nH]c2=O)O1 ; *C[C@@H]1CN(P(=O)(N=C2N(C)CCN2C)O*)C[C@H](n2ccc(N)nc2=O)O1]

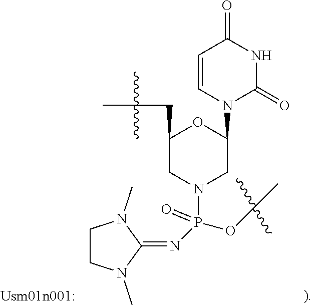 [SMILES: *C[C@@H]1CN(P(=O)(N=C2N(C)CCN2C)O*)C[C@H](n2ccc(=O)[nH]c2=O)O1]

In some embodiments, it forms

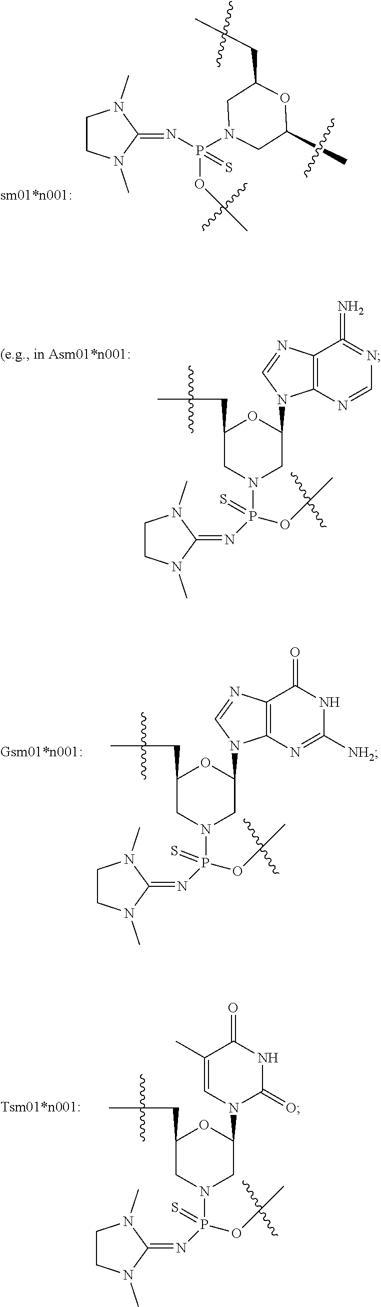

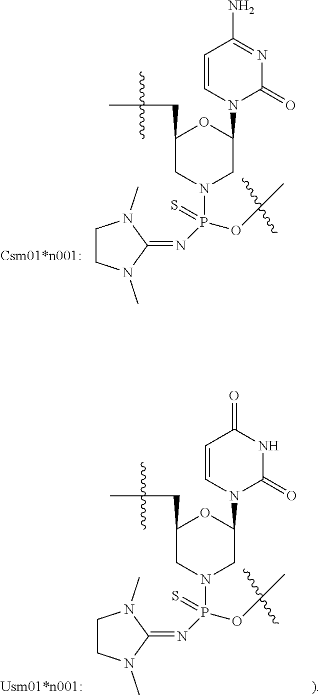 [SMILES: *C[C@@H]1CN(P(=S)(N=C2N(C)CCN2C)O*)C[C@H](n2ccc(N)nc2=O)O1 ; *C[C@@H]1CN(P(=S)(N=C2N(C)CCN2C)O*)C[C@H](n2ccc(=O)[nH]c2=O)O1]

In some embodiments, each Rp and Sp is independently the configuration of a phosphorothioate internucleotidic linkage wherein X is S. In some embodiments, each Rp and Sp is independently the configuration of a phosphorothioate internucleotidic linkage.

In some embodiments, a pattern of backbone chiral centers of an oligonucleotide or a region (e.g., of a 5′-wing) is or comprises Sp(Op)₃. In some embodiments, a pattern of backbone chiral centers of an oligonucleotide or a region (e.g., of a 5′-wing) is or comprises Rp(Op)₃. In some embodiments, a pattern of backbone chiral centers of an oligonucleotide or a region (e.g., of a 3′-wing) is or comprises (Op)₃Sp. In some embodiments, a pattern of backbone chiral centers of an oligonucleotide or a region (e.g., of a 3′-wing) is or comprises (Op)₃Rp. In some embodiments, a pattern of backbone chiral centers of an oligonucleotide or a region (e.g., of a core) is or comprises Rp(Sp)₄Rp(Sp)₄Rp. In some embodiments, a pattern of backbone chiral centers of an oligonucleotide or a region (e.g., of a core) is or comprises (Sp)₅Rp(Sp)₄Rp. In some embodiments, a pattern of backbone chiral centers of an oligonucleotide or a region (e.g., of a core) is or comprises (Sp)₅Rp(Sp)₅. In some embodiments, a pattern of backbone chiral centers of an oligonucleotide or a region (e.g., of a core) is or comprises Rp(Sp)₄Rp(Sp)₅. In some embodiments, a pattern of backbone chiral centers of an oligonucleotide is or comprises Np(Op)₃Rp(Sp)₄Rp(Sp)₄Rp(Op)₃Np. In some embodiments, a pattern of backbone chiral centers of an oligonucleotide is or comprises Np(Op)₃(Sp)₅Rp(Sp)₄Rp(Op)₃Np. In some embodiments, a pattern of backbone chiral centers of an oligonucleotide is or comprises Np(Op)₃(Sp)₅Rp(Sp)₅(Op)₃Np. In some embodiments, a pattern of backbone chiral centers of an oligonucleotide is or comprises Np(Op)₃Rp(Sp)₄Rp(Sp)₅(Op)₃Np. In some embodiments, a pattern of backbone chiral centers of an oligonucleotide is or comprises Sp(Op)₃Rp(Sp)₄Rp(Sp)₄Rp(Op)₃Sp. In some embodiments, a pattern of backbone chiral centers of an oligonucleotide is or comprises Sp(Op)₃(Sp)₅Rp(Sp)₄Rp(Op)₃Sp. In some embodiments, a pattern of backbone chiral centers of an oligonucleotide is or comprises Sp(Op)₃(Sp)₅Rp(Sp)₅(Op)₃Sp. In some embodiments, a pattern of backbone chiral centers of an oligonucleotide is or comprises Sp(Op)₃Rp(Sp)₄Rp(Sp)₅(Op)₃Sp. In some embodiments, a pattern of backbone chiral centers of an oligonucleotide is or comprises Rp(Op)₃Rp(Sp)₄Rp(Sp)₄Rp(Op)₃Rp. In some embodiments, a pattern of backbone chiral centers of an oligonucleotide is or comprises Rp(Op)₃(Sp)₅Rp(Sp)₄Rp(Op)₃Rp. In some embodiments, a pattern of backbone chiral centers of an oligonucleotide is or comprises Rp(Op)₃(Sp)₅Rp(Sp)₅(Op)₃Rp. In some embodiments, a pattern of backbone chiral centers of an oligonucleotide is or comprises Rp(Op)₃Rp(Sp)₄Rp(Sp)₅(Op)₃Rp.

In some embodiments, each of m, y, t, n, k, f, g, h, and j is independently 1-25.

In some embodiments, m is 1, 2, 3, 4, 5, 6, 7, 8, 9, 10, 11, 12, 13, 14, 15, 16, 17, 18, 19, 20, 21, 22, 23, 24, or 25. In some embodiments, in a pattern of backbone chiral centers each m is independently 2 or more. In some embodiments, each m is independently 2, 3, 4, 5, 6, 7, 8, 9, or 10. In some embodiments, each m is independently 2-3, 2-5, 2-6, or 2-10. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, where there are two or more occurrences of m, they can be the same or different, and each of them is independently as described in the present disclosure.

In some embodiments, y is 1, 2, 3, 4, 5, 6, 7, 8, 9, 10, 11, 12, 13, 14, 15, 16, 17, 18, 19, 20, 21, 22, 23, 24, or 25. In some embodiments, y is 1, 2, 3, 4, 5, 6, 7, 8, 9, or 10. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10.

In some embodiments, t is 1, 2, 3, 4, 5, 6, 7, 8, 9, 10, 11, 12, 13, 14, 15, 16, 17, 18, 19, 20, 21, 22, 23, 24, or 25. In some embodiments, each t is independently 1, 2, 3, 4, 5, 6, 7, 8, 9, or 10. In some embodiments, t is 2 or more. In some embodiments, t is 1. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, t is 9. In some embodiments, t is 10. In some embodiments, where there are two or more occurrences oft, they can be the same or different, and each of them is independently as described in the present disclosure.

In some embodiments, n is 1, 2, 3, 4, 5, 6, 7, 8, 9, 10, 11, 12, 13, 14, 15, 16, 17, 18, 19, 20, 21, 22, 23, 24, or 25. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, where there are two or more occurrences of n, they can be the same or different, and each of them is independently as described in the present disclosure. In many embodiments, in a pattern of backbone chiral centers, at least one occurrence of n is 1; in some cases, each n is 1.

In some embodiments, k is 1, 2, 3, 4, 5, 6, 7, 8, 9, 10, 11, 12, 13, 14, 15, 16, 17, 18, 19, 20, 21, 22, 23, 24, or 25. In some embodiments, k is 1. In some embodiments, k is 2. In some embodiments, k is 3. In some embodiments, k is 4. In some embodiments, k is 5. In some embodiments, k is 6. In some embodiments, k is 7. In some embodiments, k is 8. In some embodiments, k is 9. In some embodiments, k is 10.

In some embodiments, f is 1-20. In some embodiments, f is 1-10. In some embodiments, f is 1-5. In some embodiments, f is 1, 2, 3, 4, 5, 6, 7, 8, 9, 10, 11, 12, 13, 14, 15, 16, 17, 18, 19, 20, 21, 22, 23, 24, or 25. In some embodiments, f is 1. In some embodiments, f is 2. In some embodiments, f is 3. In some embodiments, f is 4. In some embodiments, f is 5. In some embodiments, f is 6. In some embodiments, f is 7. In some embodiments, f is 8. In some embodiments, f is 9. In some embodiments, f is 10.

In some embodiments, g is 1-20. In some embodiments, g is 1-10. In some embodiments, g is 1-5. In some embodiments, g is 2-10. In some embodiments, g is 2-5. In some embodiments, g is 1, 2, 3, 4, 5, 6, 7, 8, 9, 10, 11, 12, 13, 14, 15, 16, 17, 18, 19, 20, 21, 22, 23, 24, or 25. In some embodiments, g is 1. In some embodiments, g is 2. In some embodiments, g is 3. In some embodiments, g is 4. In some embodiments, g is 5. In some embodiments, g is 6. In some embodiments, g is 7. In some embodiments, g is 8. In some embodiments, g is 9. In some embodiments, g is 10.

In some embodiments, h is 1-20. In some embodiments, h is 1-10. In some embodiments, h is 1-5. In some embodiments, h is 2-10. In some embodiments, h is 2-5. In some embodiments, h is 1, 2, 3, 4, 5, 6, 7, 8, 9, 10, 11, 12, 13, 14, 15, 16, 17, 18, 19, 20, 21, 22, 23, 24, or 25. In some embodiments, h is 1. In some embodiments, his 2. In some embodiments, his 3. In some embodiments, his 4. In some embodiments, h is 5. In some embodiments, h is 6. In some embodiments, h is 7. In some embodiments, h is 8. In some embodiments, h is 9. In some embodiments, h is 10.

In some embodiments, j is 1-20. In some embodiments, j is 1-10. In some embodiments, j is 1-5. In some embodiments, j is 1, 2, 3, 4, 5, 6, 7, 8, 9, 10, 11, 12, 13, 14, 15, 16, 17, 18, 19, 20, 21, 22, 23, 24, or 25. In some embodiments, j is 1. In some embodiments, j is 2. In some embodiments, j is 3. In some embodiments, j is 4. In some embodiments, j is 5. In some embodiments, j is 6. In some embodiments, j is 7. In some embodiments, j is 8. In some embodiments, j is 9. In some embodiments, j is 10.

In some embodiments, at least one n is 1, and at least one m is no less than 2. In some embodiments, at least one n is 1, at least one t is no less than 2, and at least one m is no less than 3. In some embodiments, each n is 1. In some embodiments, t is 1. In some embodiments, at least one t >1. In some embodiments, at least one t >2. In some embodiments, at least one t >3. In some embodiments, at least one t >4. In some embodiments, at least one m >1. In some embodiments, at least one m >2. In some embodiments, at least one m >3. In some embodiments, at least one m >4. In some embodiments, a pattern of backbone chiral centers comprises one or more achiral natural phosphate linkages. In some embodiments, the sum of m, t, and n (or m and n if not in a pattern) is no less than 5, 6, 7, 8, 9, 10, 11, 12, 13, 14, 15, 16, 17, 18, 19 or 20. In some embodiments, the sum is 5. In some embodiments, the sum is 6. In some embodiments, the sum is 7. In some embodiments, the sum is 8. In some embodiments, the sum is 9. In some embodiments, the sum is 10. In some embodiments, the sum is 11. In some embodiments, the sum is 12. In some embodiments, the sum is 13. In some embodiments, the sum is 14. In some embodiments, the sum is 15.

In some embodiments, a number of linkage phosphorus in chirally controlled internucleotidic linkages are Sp. In some embodiments, at least 10%, 20%, 25%, 30%, 35%, 40%, 45%, 50%, 55%, 60%, 65%, 70%, 75%, 80%, 85%, 90% or 95% of chirally controlled internucleotidic linkages have Sp linkage phosphorus. In some embodiments, at least 10%, 20%, 25%, 30%, 35%, 40%, 45%, 50%, 55%, 60%, 65%, 70%, 75%, 80%, 85%, 90% or 95%, or 100% of chirally controlled phosphorothioate internucleotidic linkages have Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all chiral internucleotidic linkages are chirally controlled internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all chiral internucleotidic linkages are chirally controlled phosphorothioate internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all internucleotidic linkages are chirally controlled internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all phosphorothioate internucleotidic linkages are chirally controlled internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of chirally controlled non-negatively charged internucleotidic linkages (e.g., neutral internucleotidic linkages, n001, etc.) have Rp linkage phosphorus. In some embodiments, the percentage is at least 20%. In some embodiments, the percentage is at least 30%. In some embodiments, the percentage is at least 40%. In some embodiments, the percentage is at least 50%. In some embodiments, the percentage is at least 60%. In some embodiments, the percentage is at least 65%. In some embodiments, the percentage is at least 70%. In some embodiments, the percentage is at least 75%. In some embodiments, the percentage is at least 80%. In some embodiments, the percentage is at least 90%. In some embodiments, the percentage is at least 95%. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Sp linkage phosphorus. In some embodiments, at least 5 internucleotidic linkages are chirally controlled internucleotidic linkages having Sp linkage phosphorus. In some embodiments, at least 6 internucleotidic linkages are chirally controlled internucleotidic linkages having Sp linkage phosphorus. In some embodiments, at least 7 internucleotidic linkages are chirally controlled internucleotidic linkages having Sp linkage phosphorus. In some embodiments, at least 8 internucleotidic linkages are chirally controlled internucleotidic linkages having Sp linkage phosphorus. In some embodiments, at least 9 internucleotidic linkages are chirally controlled internucleotidic linkages having Sp linkage phosphorus. In some embodiments, at least 10 internucleotidic linkages are chirally controlled internucleotidic linkages having Sp linkage phosphorus. In some embodiments, at least 11 internucleotidic linkages are chirally controlled internucleotidic linkages having Sp linkage phosphorus. In some embodiments, at least 12 internucleotidic linkages are chirally controlled internucleotidic linkages having Sp linkage phosphorus. In some embodiments, at least 13 internucleotidic linkages are chirally controlled internucleotidic linkages having Sp linkage phosphorus. In some embodiments, at least 14 internucleotidic linkages are chirally controlled internucleotidic linkages having Sp linkage phosphorus. In some embodiments, at least 15 internucleotidic linkages are chirally controlled internucleotidic linkages having Sp linkage phosphorus. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In some embodiments, no more than 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In some embodiments, one and no more than one internucleotidic linkage in an oligonucleotide is a chirally controlled internucleotidic linkage having Rp linkage phosphorus. In some embodiments, 2 and no more than 2 internucleotidic linkages in an oligonucleotide are chirally controlled internucleotidic linkages having Rp linkage phosphorus. In some embodiments, 3 and no more than 3 internucleotidic linkages in an oligonucleotide are chirally controlled internucleotidic linkages having Rp linkage phosphorus. In some embodiments, 4 and no more than 4 internucleotidic linkages in an oligonucleotide are chirally controlled internucleotidic linkages having Rp linkage phosphorus. In some embodiments, 5 and no more than 5 internucleotidic linkages in an oligonucleotide are chirally controlled internucleotidic linkages having Rp linkage phosphorus. In some embodiments, each Rp chirally controlled internucleotidic linkage is independently a non-negatively charged internucleotidic linkage. In some embodiments, each Rp chirally controlled internucleotidic linkage is independently a neutral internucleotidic linkage. In some embodiments, each Rp chirally controlled internucleotidic linkage is independently n001. In some embodiments, each non-negatively charged internucleotidic linkage is n001.

In some embodiments, an oligonucleotide comprises one or more Rp internucleotidic linkages. In some embodiments, an oligonucleotide comprises one and no more than one Rp internucleotidic linkages. In some embodiments, an oligonucleotide comprises two or more Rp internucleotidic linkages. In some embodiments, an oligonucleotide comprises three or more Rp internucleotidic linkages. In some embodiments, an oligonucleotide comprises four or more Rp internucleotidic linkages. In some embodiments, an oligonucleotide comprises five or more Rp internucleotidic linkages. In some embodiments, about 5%-50% of all chirally controlled internucleotidic linkages in an oligonucleotide are Rp. In some embodiments, about 5%-40% of all chirally controlled internucleotidic linkages in an oligonucleotide are Rp. In some embodiments, about 10%-40% of all chirally controlled internucleotidic linkages in an oligonucleotide are Rp. In some embodiments, about 15%-40% of all chirally controlled internucleotidic linkages in an oligonucleotide are Rp. In some embodiments, about 20%-40% of all chirally controlled internucleotidic linkages in an oligonucleotide are Rp. In some embodiments, about 25%-40% of all chirally controlled internucleotidic linkages in an oligonucleotide are Rp. In some embodiments, about 30%-40% of all chirally controlled internucleotidic linkages in an oligonucleotide are Rp. In some embodiments, about 35%-40% of all chirally controlled internucleotidic linkages in an oligonucleotide are Rp.

In some embodiments, instead of an Rp internucleotidic linkage, a natural phosphate linkage may be similarly utilized, optionally with a modification, e.g., a sugar modification (e.g., a 5′-modification such as R^{5s} as described herein). In some embodiments, a modification improves stability of a natural phosphate linkage.

In some embodiments, at least about 25% of the internucleotidic linkages of an oligonucleotide are chirally controlled and have Sp linkage phosphorus. In some embodiments, at least about 30% of the internucleotidic linkages of an oligonucleotide are chirally controlled and have Sp linkage phosphorus. In some embodiments, at least about 40% of the internucleotidic linkages of a provided oligonucleotide are chirally controlled and have Sp linkage phosphorus. In some embodiments, at least about 50% of the internucleotidic linkages of a provided oligonucleotide are chirally controlled and have Sp linkage phosphorus. In some embodiments, at least about 60% of the internucleotidic linkages of a provided oligonucleotide are chirally controlled and have Sp linkage phosphorus. In some embodiments, at least about 65% of the internucleotidic linkages of a provided oligonucleotide are chirally controlled and have Sp linkage phosphorus. In some embodiments, at least about 70% of the internucleotidic linkages of a provided oligonucleotide are chirally controlled and have Sp linkage phosphorus. In some embodiments, at least about 75% of the internucleotidic linkages of a provided oligonucleotide are chirally controlled and have Sp linkage phosphorus. In some embodiments, at least about 80% of the internucleotidic linkages of a provided oligonucleotide are chirally controlled and have Sp linkage phosphorus. In some embodiments, at least about 85% of the internucleotidic linkages of a provided oligonucleotide are chirally controlled and have Sp linkage phosphorus. In some embodiments, at least about 90% of the internucleotidic linkages of a provided oligonucleotide are chirally controlled and have Sp linkage phosphorus. In some embodiments, at least about 95% of the internucleotidic linkages of a provided oligonucleotide are chirally controlled and have Sp linkage phosphorus.

### Additional Chemical Moieties

In some embodiments, an oligonucleotide comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of oligonucleotides, e.g., stability, half-life, activities, delivery, pharmacodynamics properties, pharmacokinetic properties, etc. In some embodiments, certain additional chemical moieties facilitate delivery of oligonucleotides to desired cells, tissues and/or organs. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides.

In some embodiments, an oligonucleotide comprises an additional chemical moiety demonstrates increased delivery to and/or activity in a tissue or an organ (e.g., eye or a part thereof) compared to a reference oligonucleotide, e.g., a reference oligonucleotide which does not have the additional chemical moiety but is otherwise identical.

In some embodiments, additional chemical moieties are carbohydrate moieties, targeting moieties, etc., which, when incorporated into oligonucleotides, can improve one or more properties. In some embodiments, an additional chemical moiety is selected from glucose, GluNAc (N-acetyl amine glucosamine) and anisamide moieties.

In some embodiments, an additional chemical moiety is a targeting moiety. In some embodiments, an additional chemical moiety is or comprises a carbohydrate moiety. In some embodiments, an additional chemical moiety is or comprises a lipid moiety. In some embodiments, an additional chemical moiety is or comprises a ligand moiety for, e.g., cell receptors such as a sigma receptor, an asialoglycoprotein receptor, etc. In some embodiments, a ligand moiety is or comprises an anisamide moiety, which may be a ligand moiety for a sigma receptor. In some embodiments, an additional chemical moiety is or comprises a ligand moiety for an asialoglycoprotein receptor. In some embodiments, a ligand is or comprises GalNAc. In some embodiments, a ligand is or comprises

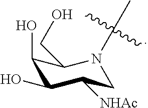

In some embodiments, an oligonucleotide comprises two or more (e.g., 2, 3, 4, 5 or more) additional moieties (e.g., GalNAc,

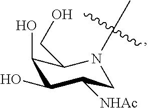

etc.)(e.g., oligonucleotides comprising Mod001, Mod155, etc.).

In some embodiments, an additional chemical moiety is or comprises a GalNac moiety. In some embodiments, an additional chemical moiety is or comprises

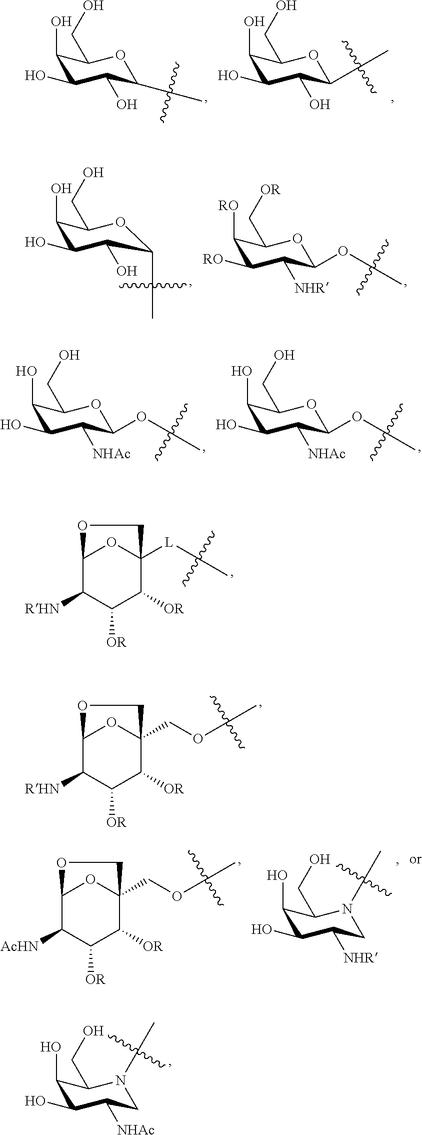

wherein each variable is independently as described in the present disclosure. In some embodiments, R is —H. In some embodiments, R′ is —C(O)R. In some embodiments, an additional chemical moiety is or comprises

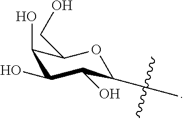

In some embodiments, an additional chemical moiety is or comprises

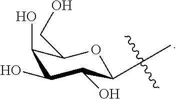

In some embodiments, an additional chemical moiety is or comprises

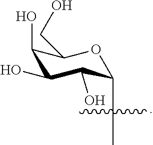

In some embodiments, an additional chemical moiety is or comprises

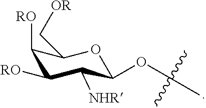

In some embodiments, an additional chemical moiety is or comprises optionally substituted

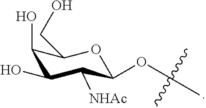

In some embodiments, an additional chemical moiety is or comprises

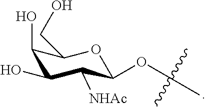

In some embodiments, an additional chemical moiety is or comprises

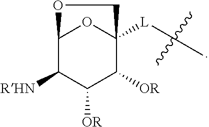

In some embodiments, an additional chemical moiety is or comprises

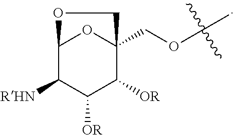

In some embodiments, an additional chemical moiety is or comprises

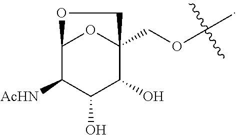

In some embodiments, an additional chemical moiety is or comprises

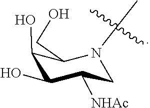

In some embodiments, an additional chemical moiety is or comprises

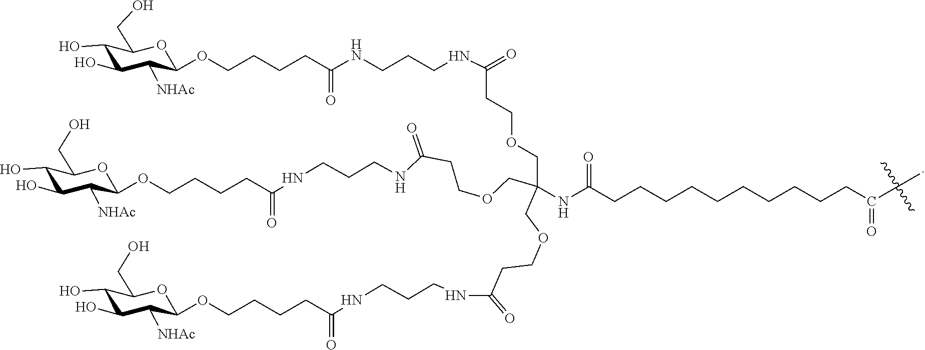

In some embodiments, an additional chemical moiety is or comprises

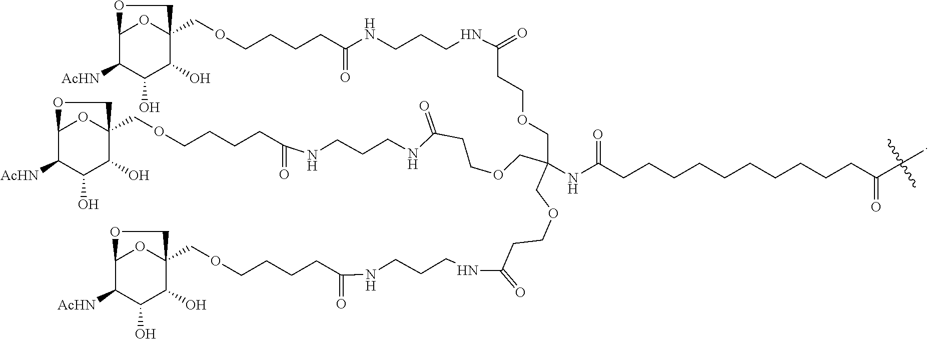

In some embodiments, an additional chemical moiety is or comprises

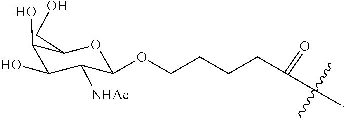

In some embodiments, an additional chemical moiety is or comprises

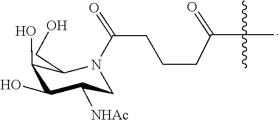

In some

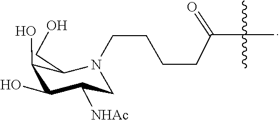

embodiments, an additional chemical moiety is or comprises In some embodiments, an additional chemical moiety is or comprises

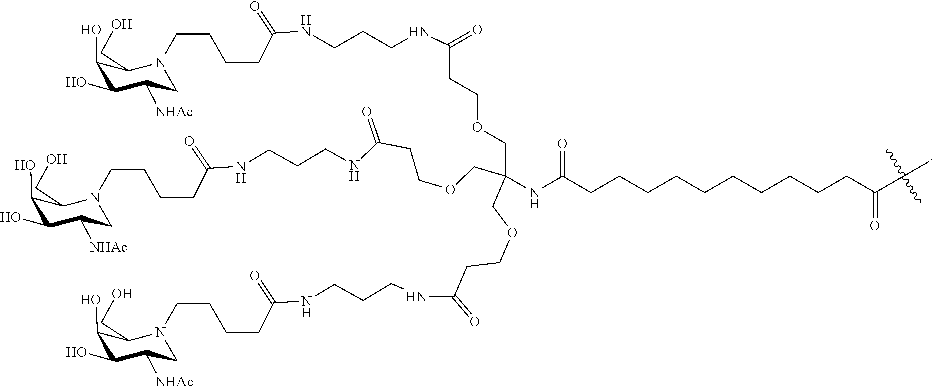

In some embodiments, an additional moiety is or comprises:

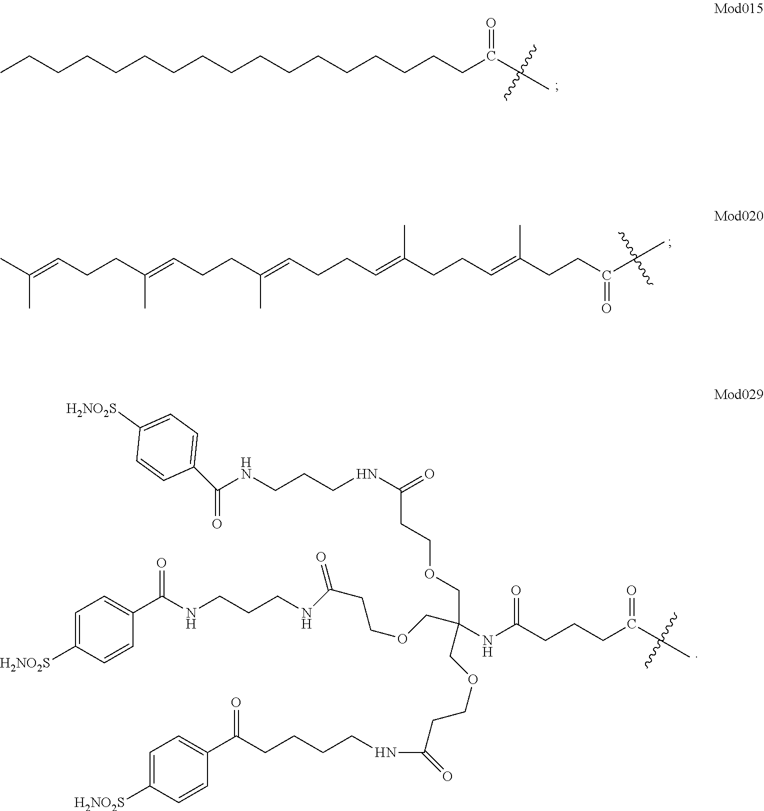 [SMILES: *C(=O)CCCCCCCCCCCCCCCCC ; *C(=O)CC/C(C)=C/CC/C(C)=C/CC/C=C(\C)CC/C=C(\C)CCC=C(C)C ; *C(=O)CCCC(=O)NC(COCCC(=O)NCCCCC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, an additional chemical moiety is or comprises a hydrocarbon moiety. In some embodiments, an additional chemical moiety is or comprises a hydrophobic moiety. In some embodiments, an additional chemical moiety is or comprises a lipid moiety. In some embodiments, a hydrocarbon, hydrophobic or lipid moiety is C₁₋₁₀₀, e.g., about C₅, C₆, C₇, C₈, C₉, C₁₀, C₁₁, ^{Cu}, C₁₃, C₁₄, C₁₅ to about C₁₅, C₁₆, C₁₇, C₁₈, C₁₉, C₂₀, C₂₅, C₃₅, C₄₀, C₄₅, or C₅₀, or about C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₅, C₃₅, C₄₀, C₄₅, or C₅₀ optionally substituted aliphatic. In some embodiments, it is linear. In some embodiments, it is branched. In some embodiments, it comprises no cyclic moieties. In some embodiments, it is saturated. In some embodiments, it comprises one or more unsaturation. In some embodiments, it is CH₃—(CH₂)₁₁—.

Additional moieties can be connected to oligonucleotide chains at various locations optionally through linker moieties. In some embodiments, e.g., as in WV-28763, additional moieties are connected to 5′-end of an oligonucleotide chain through linkers (e.g., L009 and n009). In some embodiments, an additional moiety may comprise one or more individual target, carbohydrate, lipid, and/or hydrocarbon moieties, each of which may be the same or different (e.g., see WV-28763).

In some embodiments, an additional moiety is or comprises one or more moieties each of which independently has the structure of a non-negatively charged internucleotidic linkage or neutral internucleotidic linkage (e.g., n001),In some embodiments, an additional moiety is or comprises

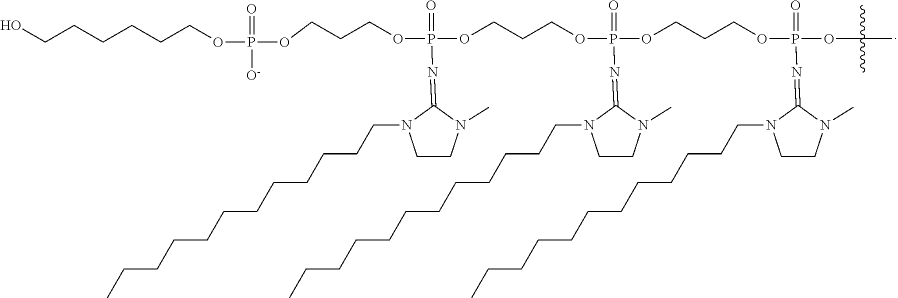

In some embodiments, an additional moiety is or comprises

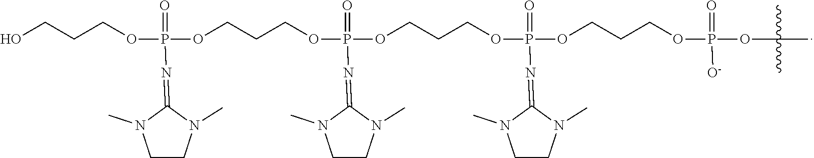

In some embodiments, an additional moiety is or comprises

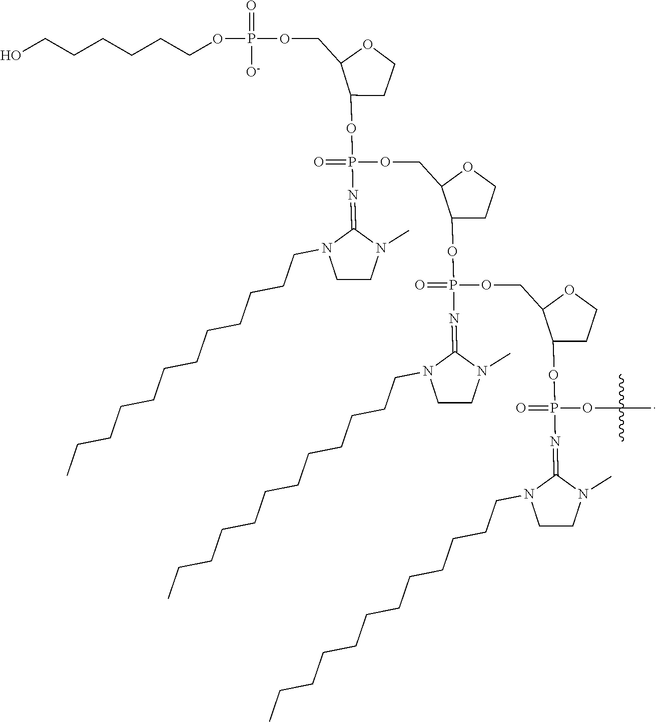

In some embodiments, an additional moiety is or comprises

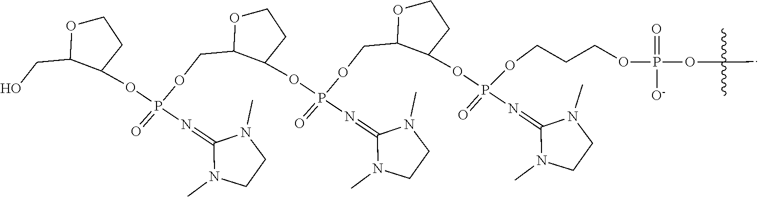

In some embodiments, an additional moiety is or comprises

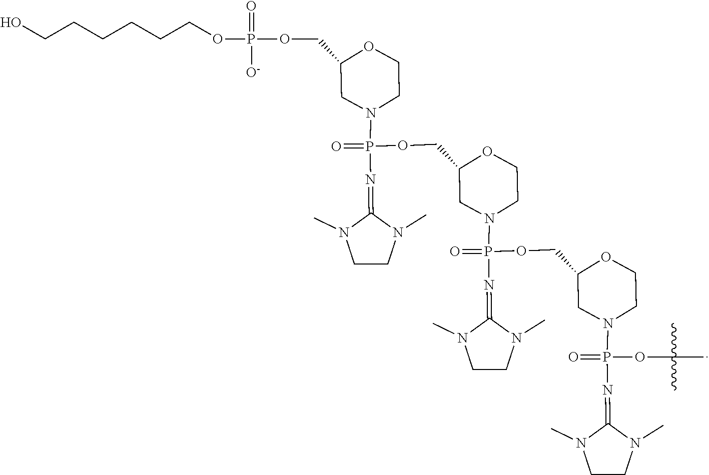

In some embodiments, an additional moiety is or comprises

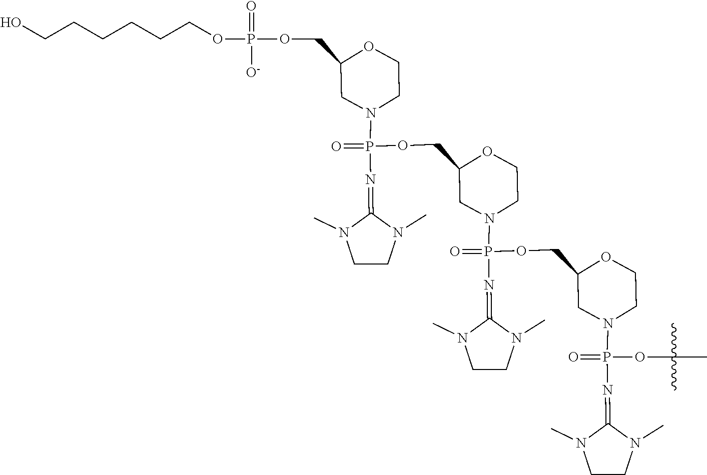 [SMILES: *OP(=O)(N=C1N(C)CCN1C)N1CCO[C@H](COP(=O)(N=C2N(C)CCN2C)N2CCO[C@H](COP(=O)(N=C3N(C)CCN3C)N3CCO[C@H](COP(=O)([O-])OCCCCCCO)C3)C2)C1]

In some embodiments, an additional moiety is or comprises

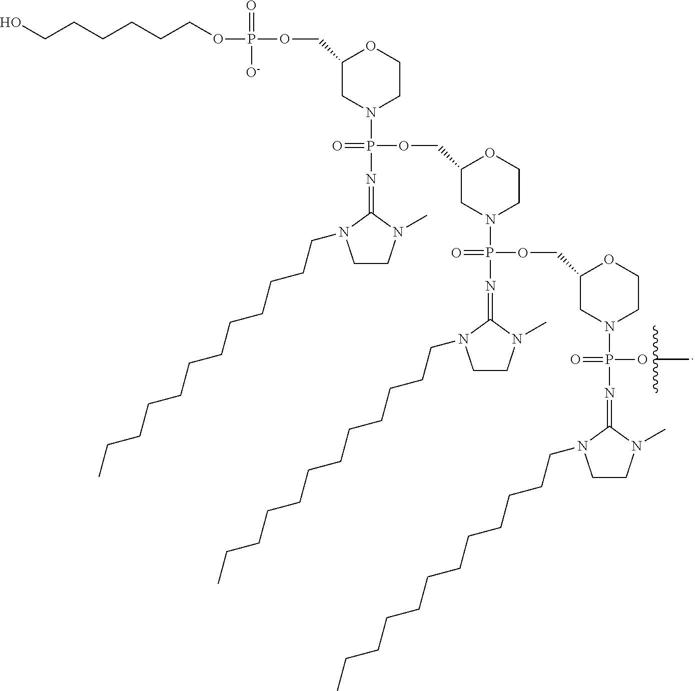

In some embodiments, an additional moiety is or comprises

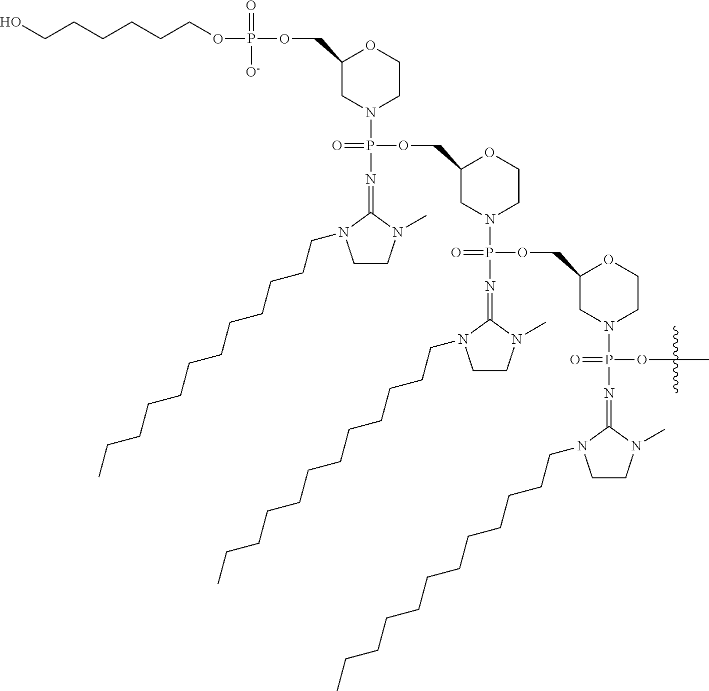 [SMILES: *OP(=O)(N=C1N(C)CCN1CCCCCCCCCCCC)N1CCO[C@H](COP(=O)(N=C2N(C)CCN2CCCCCCCCCCCC)N2CCO[C@H](COP(=O)(N=C3N(C)CCN3CCCCCCCCCCCC)N3CCO[C@H](COP(=O)([O-])OCCCCCCO)C3)C2)C1]

Certain useful additional chemical moieties are described in U.S. Pat. Nos. 9,394333, 9,744,183, 9,605,019, 9,598,458, 9,982,257, 10,160,969, 10,479995, US 2020/0056173, US 2018/0216107, US 2019/0127733, U.S. Pat. No. 10,450568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the additional chemical moieties, and connections and uses thereof, of each of which are independently incorporated herein by reference.

In some embodiments, an additional chemical moiety is cleaved from the remainder of an oligonucleotide, e.g., an oligonucleotide chain, e.g., after administration to a system, cell, tissue, organ, subject, etc. In some embodiments, additional chemical moieties promote, increase, and/or accelerate delivery to certain cells, and after delivery of oligonucleotides into such cells, additional chemical moieties are cleaved from oligonucleotides. In some embodiments, linker moieties comprise one or more cleavable moieties that can be cleaved at desirable locations (e.g., within certain type of cells, subcellular compartments such as lysosomes, etc.) and/or timing. In some embodiments, a cleavable moiety is selectively cleaved by a polypeptide, e.g., an enzyme such as a nuclease. Many useful cleavable moieties and cleavable linkers are reported and can be utilized in accordance with the present disclosure. In some embodiments, a cleavable moiety is or comprises one or more functional groups selected from amide, ester, ether, phosphodiester, disulfide, carbamate, etc. In some embodiments, a linker is as described in WO 2012/030683, WO 2021/030778, WO 2020/154344, WO 2020/154343, WO 2020/154342, WO 2020/165077, WO 2020/201406, WO 2020/216637, or WO 2020/252376.

In some embodiments, as demonstrated herein, additional chemical moieties are connected to oligonucleotide chains through linkers, e.g., L001, L009, L016, L017, L018, L019, L023, or L as described herein. In some embodiments, a linker is or comprises:

L012:-CH₂CH₂OCH₂CH₂OCH₂CH₂—. When L012 is present in the middle of an oligonucleotide, each of its two ends is independently bonded to an internucleotidic linkage (e.g., a phosphate linkage (O or PO) or a phosphorothioate linkage (can be either not chirally controlled or chirally controlled (Sp or Rp)));

L022:

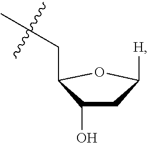

wherein L022 is connected to the rest of a molecule through a phosphate unless indicated otherwise;

L025:

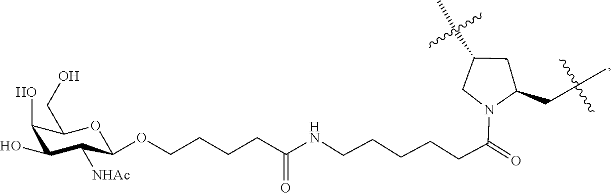

wherein the —CH₂—connection site is utilized as a C5 connection site of a sugar (e.g., a DNA sugar) and is connected to another unit (e.g., 3′ of a sugar), and the connection site on the ring is utilized as a C3 connection site and is connected to another unit (e.g., a 5′-carbon of a carbon), each of which is independently, e.g., via a linkage (e.g., a phosphate linkage (O or PO) or a phosphorothioate linkage (can be either not chirally controlled or chirally controlled (Sp or Rp))). When L025 is at a5′-end without any modifications, its —CH₂—connection site is bonded to —OH. For example, L025L025L025—in various oligonucleotides has the structure of

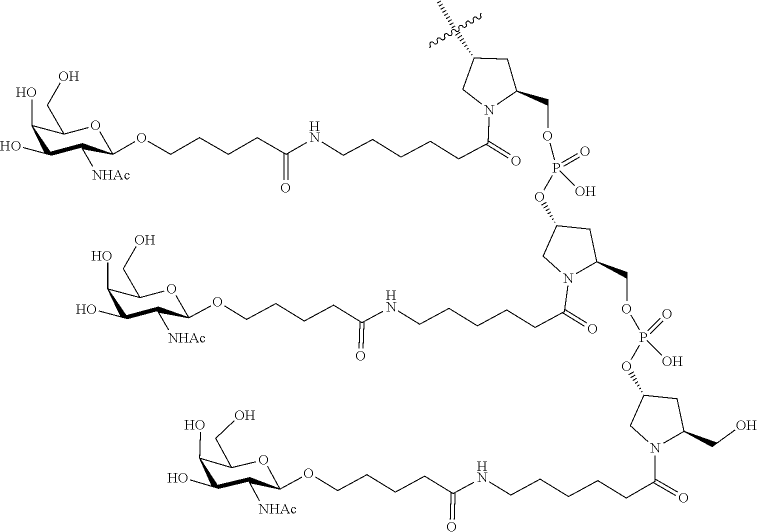

(may exist as various salt forms) and is connected to 5′-carbon of an oligonucleotide chain via a linkage as indicated (e.g., a phosphate linkage (O or PO) or a phosphorothioate linkage (can be either not chirally controlled or chirally controlled (Sp or Rp))).

### Oligonucleotides

Among other things, the present disclosure provides oligonucleotides of various designs, which may comprises various nucleobase, sugar, and/or internucleotidic linkage modifications and patterns thereof, and/or various additional chemical moieties and patterns thereof. For example, in some embodiments, provided oligonucleotides comprise sugars comprising nitrogen and modified internucleotidic linkages bonded to such nitrogen. In some embodiments, provided oligonucleotide comprise acyclic sugars. In some embodiments, provided oligonucleotides comprise patterns of modifications (e.g., of sugar and/or internucleotidic linkage modifications) and/or patters of backbone chiral centers as described herein. In some embodiments, provided oligonucleotides have base sequences that are antisense to target nucleic acids. In some embodiments, provided oligonucleotides are single-stranded. In some embodiments, provided oligonucleotides are double-stranded, e.g., siRNAs. Provided oligonucleotides and compositions thereof may be utilized for many purposes and function through various mechanisms. In some embodiments, they can reduce levels, expression, activities, etc. of target nucleic acids and/or products thereof (e.g., through RNase H, RNAi, etc.). In some embodiments, they can increase levels, expression, activities, etc. of desired target nucleic acids and/or products thereof (e.g., through exon skipping, exon inclusion, editing, etc.).

In some embodiments, provided oligonucleotides comprise at least one natural phosphate linkage and at least one modified internucleotidic linkage. In some embodiments, provided oligonucleotides comprise at least one natural phosphate linkage and at least two modified internucleotidic linkages. In some embodiments, provided oligonucleotides comprise at least one natural phosphate linkage and at least three modified internucleotidic linkages. In some embodiments, provided oligonucleotides comprise at least one natural phosphate linkage and at least four modified internucleotidic linkages. In some embodiments, provided oligonucleotides comprise at least one natural phosphate linkage and at least five modified internucleotidic linkages. In some embodiments, provided oligonucleotides comprise at least one natural phosphate linkage and 1, 2, 3, 4, 5, 6, 7, 8, 9, 10, 11, 12, 13, 14, 15, 16, 17, 18, 19, 20, 21, 22, 23, 24, or 25 modified internucleotidic linkages. In some embodiments, a modified internucleotidic linkage is a phosphorothioate internucleotidic linkage. In some embodiments, each modified internucleotidic linkage is a phosphorothioate internucleotidic linkage. In some embodiments, a modified internucleotidic linkage is a phosphorothioate triester internucleotidic linkage. In some embodiments, each modified internucleotidic linkage is a phosphorothioate triester internucleotidic linkage. In some embodiments, provided oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive modified internucleotidic linkages. In some embodiments, provided oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate internucleotidic linkages. In some embodiments, provided oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate triester internucleotidic linkages.

In some embodiments, an oligonucleotide is chirally controlled. In some embodiments, an oligonucleotide is chirally pure (or “stereopure”, “stereochemically pure”), wherein the oligonucleotide exists as a single stereoisomeric form (in many cases a single diastereoisomeric (or “diastereomeric”) form as multiple chiral centers may exist in an oligonucleotide, e.g., at linkage phosphorus, sugar carbon, etc.). As appreciated by those skilled in the art, a chirally pure oligonucleotide is separated from its other stereoisomeric forms (to the extent that some impurities may exist as chemical and biological processes, selectivities and/or purifications etc. rarely, if ever, go to absolute completeness). In a chirally pure oligonucleotide, each chiral center is independently defined with respect to its configuration (for a chirally pure oligonucleotide, each internucleotidic linkage is independently stereodefined or chirally controlled). In contrast to chirally controlled and chirally pure oligonucleotides which comprise stereodefined linkage phosphorus, racemic (or “stereorandom”, “non-chirally controlled”) oligonucleotides comprising chiral linkage phosphorus, e.g., from traditional phosphoramidite oligonucleotide synthesis without stereochemical control during coupling steps in combination with traditional sulfurization (creating stereorandom phosphorothioate internucleotidic linkages), refer to a random mixture of various stereoisomers (typically diastereoisomers (or “diastereomers”) as there are multiple chiral centers in an oligonucleotide; e.g., from traditional oligonucleotide preparation using reagents containing no chiral elements other than those in nucleosides and linkage phosphorus). For example, for A*A*A wherein * is a phosphorothioate internucleotidic linkage (which comprises a chiral linkage phosphorus), a racemic oligonucleotide preparation includes four diastereomers [2²=4, considering the two chiral linkage phosphorus, each of which can exist in either of two configurations (Sp or Rp)]: A *S A *S A, A *S A *R A, A *R A *S A, and A *R A *R A, wherein *S represents a Sp phosphorothioate internucleotidic linkage and *R represents a Rp phosphorothioate internucleotidic linkage. For a chirally pure oligonucleotide, e.g., A *S A *S A, it exists in a single stereoisomeric form and it is separated from the other stereoisomers (e.g., the diastereomers A *S A *R A, A *R A *S A, and A *R A *R A). In some embodiments, a Sp phosphorothioate is rendered as *S or * S. In some embodiments, a Rp phosphorothioate is rendered as *R or * R.

In some embodiments, provided oligonucleotides comprise 2-30 chirally controlled internucleotidic linkages. In some embodiments, provided oligonucleotides comprise 5-30 chirally controlled internucleotidic linkages. In some embodiments, provided oligonucleotides comprise 10-30 chirally controlled internucleotidic linkages. In some embodiments, provided oligonucleotides comprise 1, 2, 3, 4, 5, 6, 7, 8, 9, 10, 11, 12, 13, 14, 15, 16, 17, 18, 19, 20, 21, 22, 23, 24, or 25 or more chirally controlled internucleotidic linkages. In some embodiments, about 1-100% of all internucleotidic linkages are chirally controlled internucleotidic linkages. In some embodiments, a percentage is about 5%-100%. In some embodiments, a percentage is at least 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%. In some embodiments, a percentage is about 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%.

In some embodiments, stereochemistry of linkage phosphorus can be controlled during oligonucleotide synthesis, e.g., at couple steps. In some embodiments, a coupling step has a stereoselectivity (diastereoselectivity when there are other chiral centers) of 60% at the linkage phosphorus. After such a coupling step, the new internucleotidic linkage formed may be referred to have a 60% stereochemical purity (for oligonucleotides, typically diastereomeric purity in view of the existence of other chiral centers). In some embodiments, each coupling step independently has a stereoselectivity of at least 60%. In some embodiments, each coupling step independently has a stereoselectivity of at least 70%. In some embodiments, each coupling step independently has a stereoselectivity of at least 80%. In some embodiments, each coupling step independently has a stereoselectivity of at least 85%. In some embodiments, each coupling step independently has a stereoselectivity of at least 90%. In some embodiments, each coupling step independently has a stereoselectivity of at least 91%. In some embodiments, each coupling step independently has a stereoselectivity of at least 92%. In some embodiments, each coupling step independently has a stereoselectivity of at least 93%. In some embodiments, each coupling step independently has a stereoselectivity of at least 94%. In some embodiments, each coupling step independently has a stereoselectivity of at least 95%. In some embodiments, each coupling step independently has a stereoselectivity of at least 96%. In some embodiments, each coupling step independently has a stereoselectivity of at least 97%. In some embodiments, each coupling step independently has a stereoselectivity of at least 98%. In some embodiments, each coupling step independently has a stereoselectivity of at least 99%. In some embodiments, each coupling step independently has a stereoselectivity of at least 99.5%. In some embodiments, each coupling step independently has a stereoselectivity of virtually 100%. In some embodiments, a coupling step has a stereoselectivity of virtually 100% in that each detectable product from the coupling step analyzed by an analytical method (e.g., NMR, HPLC, etc.) has the intended stereoselectivity. In some embodiments, a chirally controlled internucleotidic linkage is typically formed with a stereoselectivity of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%). In some embodiments, a chirally controlled internucleotidic linkage has a stereochemical purity (typically diastereomeric purity for oligonucleotides with multiple chiral centers) of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus. In some embodiments, each chirally controlled internucleotidic linkage independently has a stereochemical purity (typically diastereomeric purity for oligonucleotides with multiple chiral centers) of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus. In some embodiments, a non-chirally controlled internucleotidic linkage is typically formed with a stereoselectivity of less than 60%, 70%, 80%, 85%, or 90% (in some embodiments, less than 60%; in some embodiments, less than 70%; in some embodiments, less than 80%; in some embodiments, less than 85%; in some embodiments, less than 90%). In some embodiments, each non-chirally controlled internucleotidic linkage is independently formed with a stereoselectivity of less than 60%, 70%, 80%, 85%, or 90% (in some embodiments, less than 60%; in some embodiments, less than 70%; in some embodiments, less than 80%; in some embodiments, less than 85%; in some embodiments, less than 90%). In some embodiments, a non-chirally controlled internucleotidic linkage has a stereochemical purity (typically diastereomeric purity for oligonucleotides with multiple chiral centers) of less than 60%, 70%, 80%, 85%, or 90% (in some embodiments, less than 60%; in some embodiments, less than 70%; in some embodiments, less than 80%; in some embodiments, less than 85%; in some embodiments, less than 90%) at its chiral linkage phosphorus. In some embodiments, each non-chirally controlled internucleotidic linkage independently has a stereochemical purity (typically diastereomeric purity for oligonucleotides with multiple chiral centers) of less than 60%, 70%, 80%, 85%, or 90% (in some embodiments, less than 60%; in some embodiments, less than 70%; in some embodiments, less than 80%; in some embodiments, less than 85%; in some embodiments, less than 90%) at its chiral linkage phosphorus.

In some embodiments, at least 1, 2, 3, 4, 5, 6, 7, 8, 9, or 10 couplings of a monomer (as appreciated by those skilled in the art in many embodiments a phosphoramidite for oligonucleotide synthesis) independently have a stereoselectivity less than about 60%, 70%, 80%, 85%, or 90% [for oligonucleotide synthesis, typically diastereoselectivity with respect to formed linkage phosphorus chiral center(s)]. In some embodiments, at least one coupling has a stereoselectivity less than about 60%, 70%, 80%, 85%, or 90%. In some embodiments, at least two couplings independently have a stereoselectivity less than about 60%, 70%, 80%, 85%, or 90%. In some embodiments, at least three couplings independently have a stereoselectivity less than about 60%, 70%, 80%, 85%, or 90%. In some embodiments, at least four couplings independently have a stereoselectivity less than about 60%, 70%, 80%, 85%, or 90%. In some embodiments, at least five couplings independently have a stereoselectivity less than about 60%, 70%, 80%, 85%, or 90%. In some embodiments, each coupling independently has a stereoselectivity less than about 60%, 70%, 80%, 85%, or 90%. In some embodiments, each non-chirally controlled internucleotidic linkage is independently formed with a stereoselectivity less than about 60%, 70%, 80%, 85%, or 90%. In some embodiments, a stereoselectivity is less than about 60%. In some embodiments, a stereoselectivity is less than about 70%. In some embodiments, a stereoselectivity is less than about 80%. In some embodiments, a stereoselectivity is less than about 90%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 90%. In some embodiments, at least one coupling has a stereoselectivity less than about 90%. In some embodiments, at least two couplings have a stereoselectivity less than about 90%. In some embodiments, at least three couplings have a stereoselectivity less than about 90%. In some embodiments, at least four couplings have a stereoselectivity less than about 90%. In some embodiments, at least five couplings have a stereoselectivity less than about 90%. In some embodiments, each coupling independently has a stereoselectivity less than about 90%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 85%. In some embodiments, each coupling independently has a stereoselectivity less than about 85%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 80%. In some embodiments, each coupling independently has a stereoselectivity less than about 80%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 70%. In some embodiments, each coupling independently has a stereoselectivity less than about 70%.

In some embodiments, a stereochemical purity, e.g., diastereomeric purity, is about 60%-100%. In some embodiments, a diastereomeric purity, is about 60%-100%. In some embodiments, diastereomeric purity of chirally controlled linkage phosphorus is about 60%-100%, typically 85%-100% or 90%-100%. In some embodiments, diastereomeric purity of chirally controlled phosphorothioate internucleotidic linkages is about 90%-100%. In some embodiments, the percentage is at least 60%, 65%, 70%, 75%, 80%, 85%, 90%, 91%, 92%, 93%, 93%, 95%, 96%, 97%, 98%, or 99%. In some embodiments, the percentage is at least 80%, 85%, 90%, 91%, 92%, 93%, 93%, 95%, 96%, 97%, 98%, or 99%. In some embodiments, the percentage is at least 90%, 91%, 92%, 93%, 93%, 95%, 96%, 97%, 98%, or 99%. In some embodiments, a diastereomeric purity is at least 60%. In some embodiments, a diastereomeric purity is at least 70%. In some embodiments, a diastereomeric purity is at least 80%. In some embodiments, a diastereomeric purity is at least 85%. In some embodiments, a diastereomeric purity is at least 90%. In some embodiments, a diastereomeric purity is at least 91%. In some embodiments, a diastereomeric purity is at least 92%. In some embodiments, a diastereomeric purity is at least 93%. In some embodiments, a diastereomeric purity is at least 94%. In some embodiments, a diastereomeric purity is at least 95%. In some embodiments, a diastereomeric purity is at least 96%. In some embodiments, a diastereomeric purity is at least 97%. In some embodiments, a diastereomeric purity is at least 98%. In some embodiments, a diastereomeric purity is at least 99%. In some embodiments, a diastereomeric purity is at least 99.5%. As understood by a person having ordinary skill in the art, in some embodiments, diastereoselectivity of a coupling or diastereomeric purity of a chiral linkage phosphorus center can be assessed through the diastereoselectivity of a dimer formation or diastereomeric purity of a dimer prepared under the same or comparable conditions, wherein the dimer has the same nucleosides and internucleotidic linkage.

In some embodiments, an oligonucleotide comprises a chiral auxiliary, which, for example, a chiral auxiliary used to control the stereoselectivity of a reaction, e.g., a coupling reaction in an oligonucleotide synthesis cycle. In some embodiments, an internucleotidic linkage comprises a chiral auxiliary.

Various technologies can be utilized for identifying or confirming stereochemistry of chiral elements (e.g., configuration of chiral linkage phosphorus) and/or patterns of backbone chiral centers, and/or for assessing stereoselectivity (e.g., diastereoselectivity of couple steps in oligonucleotide synthesis) and/or stereochemical purity (e.g., diastereomeric purity of internucleotidic linkages, compounds (e.g., oligonucleotides), etc.). Example technologies include NMR [e.g., 1D (one-dimensional) and/or 2D (two-dimensional) ¹H-³¹P HETCOR (heteronuclear correlation spectroscopy)], HPLC, RP-HPLC, mass spectrometry, LC-MS, and cleavage of internucleotidic linkages by stereospecific nucleases, etc., which may be utilized individually or in combination. Example useful nucleases include benzonase, micrococcal nuclease, and svPDE (snake venom phosphodiesterase), which are specific for certain internucleotidic linkages with Rp linkage phosphorus (e.g., a Rp phosphorothioate linkage); and nuclease P1, mung bean nuclease, and nuclease S1, which are specific for internucleotidic linkages with Sp linkage phosphorus (e.g., a Sp phosphorothioate linkage). Without wishing to be bound by any particular theory, the present disclosure notes that, in at least some cases, cleavage of oligonucleotides by a particular nuclease may be impacted by structural elements, e.g., chemical modifications (e.g., 2′-modifications of a sugars), base sequences, or stereochemical contexts. For example, it is observed that in some cases, benzonase and micrococcal nuclease, which are specific for internucleotidic linkages with Rp linkage phosphorus, were unable to cleave an isolated Rp phosphorothioate internucleotidic linkage flanked by Sp phosphorothioate internucleotidic linkages.

In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, the solid support is Highly Crosslinked Polystyrene (HCP). In some embodiments, the solid support is hybrid support of Controlled Pore Glass (CPG) and Highly Cross-linked Polystyrene (HCP),In some embodiments, a solid support is a metal foam. In some embodiments, a solid support is a resin. In some embodiments, oligonucleotides are cleaved from a solid support.

As used in the present disclosure, in some embodiments, “one or more” is 1-200, 1-150, 1-100, 1-90, 1-80, 1-70, 1-60, 1-50, 1-40, 1-30, or is or is about 1, 2, 3, 4, 5, 6, 7, 8, 9, 10, 11, 12, 13, 14, 15, 16, 17, 18, 19, 20, 21, 22, 23, 24, or 25. In some embodiments, “one or more” is one. In some embodiments, “one or more” is two. In some embodiments, “one or more” is three. In some embodiments, “one or more” is four. In some embodiments, “one or more” is five. In some embodiments, “one or more” is six. In some embodiments, “one or more” is seven. In some embodiments, “one or more” is eight. In some embodiments, “one or more” is nine. In some embodiments, “one or more” is ten. In some embodiments, “one or more” is at least one. In some embodiments, “one or more” is at least two. In some embodiments, “one or more” is at least three. In some embodiments, “one or more” is at least four. In some embodiments, “one or more” is at least five. In some embodiments, “one or more” is at least six. In some embodiments, “one or more” is at least seven. In some embodiments, “one or more” is at least eight. In some embodiments, “one or more” is at least nine. In some embodiments, “one or more” is at least ten.

As used in the present disclosure, in some embodiments, “at least one” is 1-200, 1-150, 1-100, 1-90, 1-80, 1-70, 1-60, 1-50, 1-40, 1-30, or is or is about 1, 2, 3, 4, 5, 6, 7, 8, 9, 10, 11, 12, 13, 14, 15, 16, 17, 18, 19, 20, 21, 22, 23, 24, or 25. In some embodiments, “at least one” is one. In some embodiments, “at least one” is two. In some embodiments, “at least one” is three. In some embodiments, “at least one” is four. In some embodiments, “at least one” is five. In some embodiments, “at least one” is six. In some embodiments, “at least one” is seven. In some embodiments, “at least one” is eight. In some embodiments, “at least one” is nine. In some embodiments, “at least one” is ten.

In some embodiments, oligonucleotides are provided as salt forms. In some embodiments, oligonucleotides are provided as salts comprising negatively-charged internucleotidic linkages (e.g., phosphorothioate internucleotidic linkages, natural phosphate linkages, etc.) existing as their salt forms. In some embodiments, oligonucleotides are provided as pharmaceutically acceptable salts. In some embodiments, oligonucleotides are provided as metal salts. In some embodiments, oligonucleotides are provided as sodium salts. In some embodiments, oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

In some embodiments, oligonucleotides in compositions comprise 1, 2, 3, 4, 5, 6, 7, 8, 9, 10 or more stereorandom internucleotidic linkages (mixture of Rp and Sp linkage phosphorus at the internucleotidic linkage, e.g., from traditional non-chirally controlled oligonucleotide synthesis). In some embodiments, oligonucleotides comprise one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chirally controlled internucleotidic linkages (Rp or Sp linkage phosphorus at the internucleotidic linkage, e.g., from chirally controlled oligonucleotide synthesis). In some embodiments, an internucleotidic linkage is a phosphorothioate internucleotidic linkage. In some embodiments, an internucleotidic linkage is a stereorandom phosphorothioate internucleotidic linkage. In some embodiments, an internucleotidic linkage is a chirally controlled phosphorothioate internucleotidic linkage. In some embodiments, each phosphorothioate internucleotidic linkage is independently chirally controlled.

In some embodiments, a compound, e.g., an oligonucleotide, has the structure of: or a salt thereof, wherein:

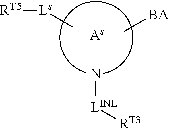

BA is an optionally substituted or protected nucleobase;

R^{T5} is optionally substituted or protected hydroxyl, an optionally substituted or protected nucleotide moiety, an oligonucleotide moiety, R′, or an additional chemical moiety optionally connected through a linker;

R^{T3} is hydrogen, an optionally substituted or protected or nucleoside nucleotide moiety, an oligonucleotide moiety, R′, or an additional chemical moiety optionally connected through a linker;

L^{INL} is —Y—P^{L}(—X—R^{L})—Z—, —C(O)—O— wherein —C(O)— in bonded to a nitrogen atom,

—C(O)—N(R′)—, or -L^{L1}-Cy^{1L}-L^{L2}-,

P^{L} is P, P(═W), P->B(-L^{L}-R^{L})₃, or P^{N};

W is O, N(-L^{L}-R^{L}), S or Se;

P^{N} is P═N—C(-L^{L}-R′)(=L^{N}-R′) or P═N-L^{L}-R^{L};

L^{N} is ═N-L^{L1}-, ═CH-L^{L1}- wherein CH is optionally substituted, or ═N⁺(R′)(Q⁻)-L^{L1}-;

Q⁻ is an anion;

each of X, Y and Z is independently —O—, —S—, —N(-L^{L}-R^{L})-, or L^{L};

each R^{L} is independently -L^{L}-R′ or —N═C(-L^{L}-R′)₂;

Ring A^{s} is an optionally substituted 3-30 membered, monocyclic, bicyclic or polycyclic ring having, in addition to the nitrogen, 0-10 heteroatoms;

each of L^{s}, L^{L1}, L^{L2} and L^{L} is independently L;

-Cy^{IL}- is -Cy-;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)—, —OP(SR′)—, —OP(NR′)—, —OP(R′)—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a compound, e.g., an oligonucleotide, has the structure of:

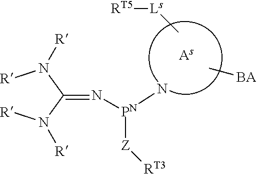

or a salt thereof, wherein each variable is independently as described herein. In some embodiments, a compound, e.g., an oligonucleotide. has the structure of:

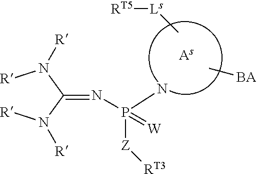

or a salt thereof, wherein each variable is independently as described herein. In some embodiments, a compound, e.g., an oligonucleotide, has the structure of:

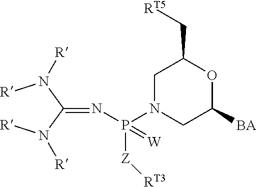

or a salt thereof, wherein each variable is independently as described herein. In some embodiments, a compound, e.g., an oligonucleotide, has the structure of

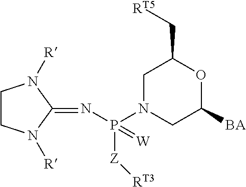

or a salt thereof.

In some embodiments, W is O. In some embodiments, W is S. In some embodiments, Z is O.

In some embodiments, R′ is optionally substituted —OH. In some embodiments, R′ is optionally substituted —OH. In some embodiments, R^{T5} is —OH. In some embodiments, R^{T5} is an optionally substituted nucleotide. In some embodiments, R^{T5} is optionally protected nucleotide. In some embodiments, R^{T5} is an optionally substituted oligonucleotide moiety. An oligonucleotide moiety may comprise one or more sugars, nucleobases and/or linkages (e.g., non-negatively charged internucleotidic linkages, phosphorothioate internucleotidic linkages, natural phosphate linkages, etc., wherein each chiral internucleotidic linkage is independently and optionally chirally controlled), and/or patterns thereof as described herein. In some embodiments, R^{T5} comprises a pattern of backbone chiral centers as described herein. In some embodiments, R⁵ comprises one or more additional chemical moieties, e.g., GalNAc. In some embodiments, R^{T5} is R′. In some embodiments, R^{T5} is a 5′-end group (e.g., those suitable for RNAi). In some embodiments, additional chemical moieties, etc., may be connected through a linker, e.g., L.

In some embodiments, R^{T3} is —H. In some embodiments, R^{T3} is R′. In some embodiments, R^{T3} is —OH. In some embodiments, R^{T3} is an optionally substituted nucleotide. In some embodiments, R^{T3} is optionally protected nucleotide. In some embodiments, R^{T3} is an optionally substituted nucleoside. In some embodiments, R^{T3} is optionally protected nucleoside. In some embodiments, R^{T3} is an optionally substituted oligonucleotide moiety. An oligonucleotide moiety may comprise one or more sugars, nucleobases and/or linkages (e.g., non-negatively charged internucleotidic linkages, phosphorothioate internucleotidic linkages, natural phosphate linkages, etc., wherein each chiral internucleotidic linkage is independently and optionally chirally controlled), and/or patterns thereof as described herein. In some embodiments, R^{T3} comprises a pattern of backbone chiral centers as described herein. In some embodiments, R⁵ comprises one or more additional chemical moieties, e.g., GalNAc. In some embodiments, R^{T3} is R′. In some embodiments, R^{T3} is a 5′-end group (e.g., those suitable for RNAi). In some embodiments, additional chemical moieties, etc., may be connected through a linker, e.g., L. In some embodiments, a nucleotide, a nucleoside, an additional chemical moiety or an oligonucleotide moiety is connected to a support, e.g., those suitable for oligonucleotide synthesis, optionally through a linker, e.g., L. In some embodiments, a support is a solid support. Certain supports and linkers as described herein.

In some embodiments, a compound, e.g., an oligonucleotide, comprises

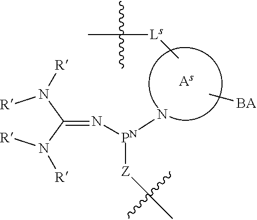

or a salt form thereof, wherein each variable is independently as described herein. In some embodiments, a compound, e.g., an oligonucleotide, comprises

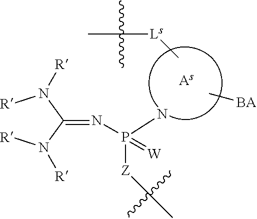

or a salt form thereof, wherein each variable is independently as described herein. In some embodiments, a compound, e.g., an oligonucleotide, comprises

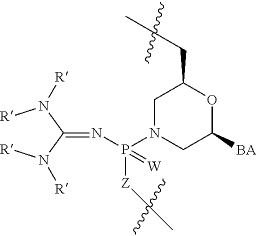

or a salt form thereof, wherein each variable is independently as described herein. In some embodiments, a compound, e.g., an oligonucleotide, has the structure of

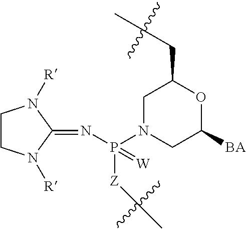

or a salt thereof. In some embodiments, W is O. In some embodiments, W is S. In some embodiments, Z is O.

In some embodiments, oligonucleotides are stereochemically pure. In some embodiments, oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75%, 80%, 85%, 90%, 95%, or 100%, or at least about 5%, 10%, 15%, 20%, 25%, 30%, 35%, 40%, 45%, 50%, 55%, 60%, 65%, 70%, 75%, 80%, 85%, 90%, 95%, or 99%, pure. In some embodiments, oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75%, 80%, 85%, 90%, 95%, or 100%, or at least about 5%, 10%, 15%, 20%, 25%, 30%, 35%, 40%, 45%, 50%, 55%, 60%, 65%, 70%, 75%, 80%, 85%, 90%, 95%, or 99%, diastereomerically pure. In some embodiments, internucleotidic linkages of oligonucleotides comprise or consist of one or more (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages, each of which independently has a diastereopurity of about or at least about 60%, 65%, 70%, 75%, 80%, 85%, 90%, 91%, 92%, 93%, 94%, 95%, 96%, 97%, 98%, 99% or 99.5%, typically about or at least about 90%, 91%, 92%, 93%, 94%, 95%, 96%, 97%, 98%, 99% or 99.5%. In some embodiments, one or more or each chirally controlled phosphorothioate internucleotidic linkage independently have a diastereomeric purity of about or at least about 90%, 91%, 92%, 93%, 94%, 95%, 96%, 97%, 98%, 99% or 99.5%. In some embodiments, one or more or each chirally controlled internucleotidic linkage having the structure of —O—P(═O)—(X-L^{L}-R^{L})—O— independently have a diastereomeric purity of about or at least about 90%, 91%, 92%, 93%, 94%, 95%, 96%, 97%, 98%, 99% or 99.5%. In some embodiments, a chiral internucleotidic linkage has a diastereopurity of at least 85%. In some embodiments, a chiral internucleotidic linkage has a diastereopurity of at least 90%. In some embodiments, a chiral internucleotidic linkage has a diastereopurity of at least 95%. In some embodiments, a chiral internucleotidic linkage has a diastereopurity of at least 96%. In some embodiments, a chiral internucleotidic linkage has a diastereopurity of at least 97%. In some embodiments, a chiral internucleotidic linkage has a diastereopurity of at least 98%. In some embodiments, a chiral internucleotidic linkage has a diastereopurity of at least 99%. In some embodiments, oligonucleotides of the present disclosure have a diastereopurity of (DS)^{CIL}, wherein DS is a diastereopurity as described in the present disclosure (e.g., 90%, 91%, 92%, 93%, 94%, 95%, 96%, 97%, 98%, 99% or 99.5% or more) and CIL is the number of chirally controlled internucleotidic linkages (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, DS is 95%-100%.

### Oligonucleotide Compositions

Among other things, the present disclosure provides various oligonucleotide compositions. In some embodiments, the present disclosure provides oligonucleotide compositions of oligonucleotides described herein. In some embodiments, an oligonucleotide composition comprises a plurality of an oligonucleotide described in the present disclosure. In some embodiments, an oligonucleotide composition is chirally controlled. In some embodiments, an oligonucleotide composition is not chirally controlled (stereorandom).

Linkage phosphorus of natural phosphate linkages is achiral. Linkage phosphorus of many modified internucleotidic linkages, e.g., phosphorothioate internucleotidic linkages, are chiral. In some embodiments, during preparation of oligonucleotide compositions (e.g., in traditional phosphoramidite oligonucleotide synthesis), configurations of chiral linkage phosphorus are not purposefully designed or controlled, creating non-chirally controlled (stereorandom) oligonucleotide compositions (substantially racemic preparations) which are complex, random mixtures of various stereoisomers (diastereoisomers)—for oligonucleotides with n chiral internucleotidic linkages (linkage phosphorus being chiral), typically 2ⁿ stereoisomers (e.g., when n is 10, 2¹⁰=1,032; when n is 20, 2²⁰=1,048,576). These stereoisomers have the same constitution, but differ with respect to the pattern of stereochemistry of their linkage phosphorus.

In some embodiments, oligonucleotide compositions are stereorandom. In some embodiments, stereorandom oligonucleotide compositions have sufficient properties and/or activities for certain purposes and/or applications. Stereoisomers within stereorandom compositions may have different properties, activities, and/or toxicities, in some instances resulting in inconsistent therapeutic effects and/or unintended side effects by stereorandom compositions, particularly compared to certain chirally controlled oligonucleotide compositions of oligonucleotides of the same constitution.

In some embodiments, oligonucleotides are chirally controlled. In some embodiments, the present disclosure provides chirally controlled oligonucleotide compositions wherein the composition comprises a non-random or controlled level of a plurality of oligonucleotides, wherein oligonucleotides of the plurality share a common base sequence, and share the same configuration of linkage phosphorus independently at 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more chiral internucleotidic linkages. In some embodiments, oligonucleotides of the plurality share the same constitution.

In some embodiments, oligonucleotides of a plurality, e.g., in provided compositions, are of the same oligonucleotide type. In some embodiments, oligonucleotides of a plurality share the same constitution. In some embodiments, oligonucleotides of a plurality are identical. As appreciated by those skilled in the art, in some embodiments, oligonucleotide of the same constitution or of the same structure may exist in different forms, e.g., in different pharmaceutically acceptable salt forms (e.g., in a liquid pharmaceutical composition comprising a buffer system whose pH is around 7.4 and/or one or more organic and/or or inorganic salts).

In some embodiments, the present disclosure encompasses technologies for designing and preparing chirally controlled oligonucleotide compositions. In some embodiments, the present disclosure provides chirally controlled oligonucleotide compositions, e.g., of many oligonucleotides in Table A1, A2, A3, and A4 which contain S and/or R in their stereochemistry/linkage. In some embodiments, a chirally controlled oligonucleotide composition comprises a controlled/pre-determined (not random as in stereorandom compositions) level of a plurality of oligonucleotides, wherein the oligonucleotides share the same linkage phosphorus stereochemistry at one or more chiral internucleotidic linkages (chirally controlled internucleotidic linkages). In some embodiments, the oligonucleotides share the same pattern of backbone chiral centers (stereochemistry of linkage phosphorus). In some embodiments, a pattern of backbone chiral centers is as described in the present disclosure.

In some embodiments, an oligonucleotide composition is a chirally controlled oligonucleotide composition comprising a plurality of oligonucleotides, wherein the oligonucleotides share:

1) a common base sequence,

2) a common pattern of backbone linkages, and

3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),

wherein the composition is enriched, relative to a substantially racemic preparation of oligonucleotides sharing the common base sequence and pattern of backbone linkages, for oligonucleotides of the plurality.

In some embodiments, an oligonucleotide composition is a chirally controlled oligonucleotide composition comprising a plurality of oligonucleotides, wherein the oligonucleotides share:

1) a common constitution, and

2) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),

wherein the composition is enriched, relative to a substantially racemic preparation of oligonucleotides sharing the common constitution for oligonucleotides of the plurality.

In some embodiments, an oligonucleotide composition is a chirally controlled oligonucleotide composition comprising a plurality of oligonucleotides, wherein the oligonucleotides share:

1) a common base sequence,

2) a common patter of backbone linkages, and

3) a common pattern of backbone chiral centers, which pattern comprises at least one Sp,

wherein the composition is enriched, relative to a substantially racemic preparation of oligonucleotides sharing the common base sequence and pattern of backbone linkages, for oligonucleotides of the plurality.

In some embodiments, an oligonucleotide composition is a chirally controlled oligonucleotide composition comprising a plurality of oligonucleotides, wherein the oligonucleotides share:

1) a common base sequence,

2) a common patter of backbone linkages, and

3) a common pattern of backbone chiral centers, which pattern comprises at least one Rp,

wherein the composition is enriched, relative to a substantially racemic preparation of oligonucleotides sharing the common base sequence and pattern of backbone linkages, for oligonucleotides of the plurality.

In some embodiments, an oligonucleotide composition is a chirally controlled oligonucleotide composition comprising a plurality of oligonucleotides, wherein the oligonucleotides share:

1) a common base sequence,

2) a common pattern of backbone linkages, and

3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),

wherein about 1-100% of all oligonucleotides within the composition that share the common constitution are the oligonucleotides of the plurality.

In some embodiments, an oligonucleotide composition is a chirally controlled oligonucleotide composition comprising a plurality of oligonucleotides, wherein the oligonucleotides share:

1) a common constitution, and

2) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),

wherein about 1-100% of all oligonucleotides within the composition that share the common constitution are the oligonucleotides of the plurality.

In some embodiments, an oligonucleotide composition is a chirally controlled oligonucleotide composition comprising a plurality of oligonucleotides, wherein the oligonucleotides share:

1) a common base sequence,

2) a common patter of backbone linkages, and

3) a common pattern of backbone chiral centers, which pattern comprises at least one Sp,

wherein about 1-100% of all oligonucleotides within the composition that share the common constitution are the oligonucleotides of the plurality.

In some embodiments, an oligonucleotide composition is a chirally controlled oligonucleotide composition comprising a plurality of oligonucleotides, wherein the oligonucleotides share:

1) a common base sequence,

2) a common patter of backbone linkages, and

3) a common pattern of backbone chiral centers, which pattern comprises at least one Rp, wherein about 1-100% of all oligonucleotides within the composition that share the common constitution are the oligonucleotides of the plurality.

In some embodiments, oligonucleotides of a plurality share the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages. In some embodiments, oligonucleotides of a plurality share the same linkage phosphorus stereochemistry at five or more (e.g., 5-50, 5-40, 5-30, 5-25, 5-20, 5-15, 5-10, 5, 6, 7, 8, 9, 10, 11, 12, 13, 14, 15, 16, 17, 18, 19, 20, or more) chiral internucleotidic linkages. In some embodiments, each chiral internucleotidic linkage is independently chirally controlled.

In some embodiments, the present disclosure provides a composition comprising a plurality of oligonucleotides, wherein each oligonucleotide of the plurality is independently a particular oligonucleotide or a salt thereof. In some embodiments, the present disclosure provides a composition comprising a plurality of oligonucleotides, wherein each oligonucleotide of the plurality is independently a particular oligonucleotide or a pharmaceutically acceptable salt thereof. In some embodiments, such a composition is enriched relative to a substantially racemic preparation of a particular oligonucleotide. As appreciated by those skilled in the art, oligonucleotides of the plurality share a common sequence which is the base sequence of the particular oligonucleotide. In some embodiments, at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the base sequence of a particular oligonucleotide are oligonucleotide of the plurality. In some embodiments, at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the constitution of a particular oligonucleotide or a salt thereof are oligonucleotide of the plurality. In some embodiments, a percentage is at least 10%. In some embodiments, a percentage is at least 20%. In some embodiments, a percentage is at least 30%. In some embodiments, a percentage is at least 40%. In some embodiments, a percentage is at least 50%. In some embodiments, it is at least 60%. In some embodiments, it is at least 70%. In some embodiments, it is at least 80%. In some embodiments, it is at least 90%. In some embodiments, it is at least 95%. In some embodiments, it is about 5-100%. In some embodiments, it is about 10-100%. In some embodiments, it is about 20-100%. In some embodiments, it is about 30-90%. In some embodiments, it is about 30-80%. In some embodiments, it is about 30-70%. In some embodiments, it is about 40-90%. In some embodiments, it is about 40-80%. In some embodiments, it is about 40-70%. In some embodiments, a particular oligonucleotide is an oligonucleotide exemplified herein, e.g., an oligonucleotide of Table Al, A2, A3, A4 or another table.

In some embodiments, an enrichment relative to a racemic preparation is that about 1-100% (e.g., about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides within the composition that share the common base sequence and pattern of backbone linkages are oligonucleotides of the plurality. In some embodiments, an enrichment relative to a racemic preparation is that about 1-100% of all oligonucleotides within the composition that share the common constitution are oligonucleotides of the plurality. In some embodiments, the present disclosure provides an oligonucleotide composition comprising an oligonucleotide, wherein about 1-100% of all oligonucleotides within the composition that share the same base sequence as the oligonucleotide share the same pattern of backbone chiral centers as the oligonucleotide. In some embodiments, the present disclosure provides an oligonucleotide composition comprising an oligonucleotide, wherein about 1-100% of all oligonucleotides within the composition that share the same base sequence as the oligonucleotide share the same oligonucleotide chain as the oligonucleotide. In some embodiments, the present disclosure provides an oligonucleotide composition comprising an oligonucleotide, wherein about 1-100% of all oligonucleotides within the composition that share the same constitution (in some embodiments, independently in various acid, base, or salt forms) as the oligonucleotide have the structure of the oligonucleotide (in some embodiments, independently in various acid, base, or salt forms). In some embodiments, the present disclosure provides an oligonucleotide composition comprising an oligonucleotide, wherein about 1-100% of all oligonucleotides within the composition that share the same base sequence as the oligonucleotide have the structure of the oligonucleotide (in some embodiments, independent in various acid, base, or salt forms). In some embodiments, a composition is a liquid composition, and oligonucleotides are dissolved in a solution.

In some embodiments, a percentage in the present disclosure, e.g., of levels of oligonucleotides in chirally controlled oligonucleotide compositions, is about, or is at least about 5%, 10%, 15%, 20%, 25%, 30%, 35%, 40%, 45%, 50%, 60%, 70%, 75%, 80%, 85%, 90%, 95%, 96%, 97%, 98%, or 99%. In some embodiments, a percentage is about, or is at least about 50%. In some embodiments, a percentage is about, or is at least about 60%. In some embodiments, a percentage is about, or is at least about 70%. In some embodiments, a percentage is about, or is at least about 75%. In some embodiments, a percentage is about, or is at least about 80%. In some embodiments, a percentage is about, or is at least about 85%. In some embodiments, a percentage is about, or is at least about 90%. In some embodiments, a percentage is about, or is at least about 95%. In some embodiments, a percentage is about, or is at least about 97%. In some embodiments, a percentage is about, or is at least about 98%. In some embodiments, a percentage is about, or is at least about 99%. As appreciated by those skilled in the art, various forms of an oligonucleotide may be properly considered to have the same constitution and/or structure, and various forms of oligonucleotides sharing the same constitution may be properly considered to have the same constitution. In some embodiments, a level as a percentage (e.g., a controlled level, a pre-determined level, an enrichment) is or is at least (DS)^{nc}, wherein DS is 90%-100%, and nc is the number of chirally controlled internucleotidic linkages as described in the present disclosure (e.g., 5, 6, 7, 8, 9, 10, 11, 12, 13, 14, 15, 16, 17, 18, 19, 20 or more). In some embodiments, each chiral internucleotidic linkage is chirally controlled, and nc is the number of chiral internucleotidic linkage. In some embodiments, DS is 90%, 91%, 92%, 93%, 94%, 95%, 96%, 97%, 98%, 99% or 99.5% or more. In some embodiments, DS is or is at least 90%. In some embodiments, DS is or is at least 91%. In some embodiments, DS is or is at least 92%. In some embodiments, DS is or is at least 93%. In some embodiments, DS is or is at least 94%. In some embodiments, DS is or is at least 95%. In some embodiments, DS is or is at least 96%. In some embodiments, DS is or is at least 97%. In some embodiments, DS is or is at least 98%. In some embodiments, DS is or is at least 99%. In some embodiments, a level (e.g., a controlled level, a pre-determined level, an enrichment) is a percentage of all oligonucleotides in a composition that share the same constitution, wherein the percentage is or is at least (DS)^{nc}. For example, when DS is 99% and nc is 10, the percentage is or is at least 90% ((⁹⁹%)¹⁰ 0.90≈90%). As appreciated by those skilled in the art, in a stereorandom preparation the percentage is typically about ½^{nc}; —when nc is 10, the percentage is about ½¹⁰≈00.001=0.1%.

In some embodiments, an oligonucleotide composition is a chirally controlled oligonucleotide composition comprising a plurality of oligonucleotides, wherein the oligonucleotides share:

1) a common base sequence,

2) a common pattern of backbone linkages, and

3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),

wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, an oligonucleotide composition is a chirally controlled oligonucleotide composition comprising a plurality of oligonucleotides, wherein the oligonucleotides share:

1) a common constitution, and

2) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),

wherein the percentage of the oligonucleotides of the plurality within all oligonucleotides in the composition that share the common constitution is at least (DS)^{nc}, wherein DS is 90%-100%, and nc is the number of chirally controlled internucleotidic linkages.

In some embodiments, an oligonucleotide composition is a chirally controlled oligonucleotide composition comprising a plurality of oligonucleotides, wherein the oligonucleotides share:

1) a common base sequence,

2) a common patter of backbone linkages, and

3) a common pattern of backbone chiral centers, which pattern comprises at least one Sp,

wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, an oligonucleotide composition is a chirally controlled oligonucleotide composition comprising a plurality of oligonucleotides, wherein the oligonucleotides share:

1) a common base sequence,

2) a common patter of backbone linkages, and

3) a common pattern of backbone chiral centers, which pattern comprises at least one Rp,

wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, the present disclosure provides a chirally controlled oligonucleotide composition comprising a plurality of oligonucleotides, wherein the oligonucleotides are structurally identical, wherein the percentage of the oligonucleotides of the plurality within all oligonucleotides of the same constitution as the oligonucleotides of the plurality in the composition is at least (DS)^{nc}, wherein DS is 90%-100%, and nc is the number of chirally controlled internucleotidic linkages.

In some embodiments, oligonucleotides of the plurality are of different salt forms. In some embodiments, oligonucleotides of the plurality comprise one or more forms, e.g., various pharmaceutically acceptable salt forms, of a single oligonucleotide. In some embodiments, oligonucleotides of the plurality comprise one or more forms, e.g., various pharmaceutically acceptable salt forms, of two or more oligonucleotides. In some embodiments, oligonucleotides of the plurality comprise one or more forms, e.g., various pharmaceutically acceptable salt forms, of 2^{NCC} oligonucleotides, wherein NCC is the number of non-chirally controlled chiral internucleotidic linkages. In some embodiments, the 2^{NCC} oligonucleotides have relatively similar levels within a composition as, e.g., none of them are specifically enriched using chirally controlled oligonucleotide synthesis.

In some embodiments, level of a plurality of oligonucleotides in a composition can be determined as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy).

In some embodiments, all chiral internucleotidic linkages are chiral controlled, and the composition is a completely chirally controlled oligonucleotide composition. In some embodiments, not all chiral internucleotidic linkages are chiral controlled internucleotidic linkages, and the composition is a partially chirally controlled oligonucleotide composition. In some embodiments, at least 10%, 20%, 30%, 40%, 50%, 60%, 70%, 80%, 85%, 90%, 91%, 92%, 93%, 94%, 95%, 96%, 97%, 98%, or 99% of all chiral internucleotidic linkages are chirally controlled. In some embodiments, at least 50%, 60%, 70%, 80%, 85%, 90%, 91%, 92%, 93%, 94%, 95%, 96%, 97%, 98%, or 99% of all chiral internucleotidic linkages are chirally controlled. In some embodiments, each phosphorothioate internucleotidic linkage is independently chirally controlled. In some embodiments, each internucleotidic linkage having the structure of —O—P^{L}(—X—R^{L})—O— is independently chirally controlled.

Oligonucleotides may comprise or consist of various patterns of backbone chiral centers (patterns of stereochemistry of chiral linkage phosphorus). Certain useful patterns of backbone chiral centers are described in the present disclosure. In some embodiments, a plurality of oligonucleotides share a common pattern of backbone chiral centers, which is or comprises a pattern described in the present disclosure (e.g., as in “Linkage Phosphorus Stereochemistry and Patterns Thereof”, a pattern of backbone chiral centers of a chirally controlled oligonucleotide in Table A1, A2, A3, and A4, etc.).

In some embodiments, a chirally controlled oligonucleotide composition is chirally pure (or stereopure, stereochemically pure) oligonucleotide composition, wherein the oligonucleotide composition comprises a plurality of oligonucleotides, wherein the oligonucleotides are identical [including that each chiral element of the oligonucleotides, including each chiral linkage phosphorus, is independently defined (stereodefined)], and the composition does not contain other stereoisomers. A chirally pure (or stereopure, stereochemically pure) oligonucleotide composition of an oligonucleotide stereoisomer does not contain other stereoisomers (as appreciated by those skilled in the art, one or more unintended stereoisomers may exist as impurities - example purities are descried in the present disclosure).

Chirally controlled oligonucleotide compositions can demonstrate a number of advantages over stereorandom oligonucleotide compositions. Among other things, chirally controlled oligonucleotide compositions are more uniform than corresponding stereorandom oligonucleotide compositions with respect to oligonucleotide structures. By controlling stereochemistry, compositions of individual stereoisomers can be prepared and assessed, so that chirally controlled oligonucleotide composition of stereoisomers with desired properties and/or activities can be developed. In some embodiments, chirally controlled oligonucleotide compositions provides better delivery, stability, clearance, activity, selectivity, and/or toxicity profiles compared to, e.g., corresponding stereorandom oligonucleotide compositions. In some embodiments, chirally controlled oligonucleotide compositions provide better efficacy, fewer side effects, and/or more convenient and effective dosage regimens. Among other things, patterns of backbone chiral centers as described herein can be utilized to provide controlled cleavage of oligonucleotide targets (e.g., transcripts such as pre-mRNA, mature mRNA, etc; including control of cleavage sites, rate and/or extent of cleavage at cleavage sites, and/or overall rate and extent of cleavage, etc.) and greatly increased target selectivity. In some embodiments, chirally controlled oligonucleotide compositions of oligonucleotides comprising certain patterns of backbone chiral centers can differentiate sequences with nucleobase difference at very few positions, in some embodiments, at single position (e.g., at SNP site, point mutation site, etc.).

As examples, certain oligonucleotides comprising certain example base sequences, nucleobase modifications and patterns thereof, sugar modifications and patterns thereof, internucleotidic linkages and patterns thereof, and/or linkage phosphorus stereochemistry and patterns thereof are presented in Table A1, A2, A3, and A4, below. In some embodiments, an oligonucleotide comprises a base sequence (or a portion thereof), one or more nucleobase modifications, a pattern of nucleobase modification (or a portion thereof), one or more sugar modifications, a pattern of sugar modification (or a portion thereof), one or more internucleotidic linkages, a pattern of internucleotidic linkage modification (or a portion thereof), a pattern of linkage phosphorus stereochemistry (or a portion thereof) of an oligonucleotide described in Table A1, A2, A3, or A4, below.

TABLE Al
Example oligonucleotides and/or compositions that target ACTB.

| | | | Stereochemistry/ |
|---|---|---|---|
| ID | Description | Base Sequence | Linkage |
| WV- | Mod001L00IfAn001fC*SfA*SfU*SfA*SfA*SfU*SfU*SfU*SfA*SfC* | ACAUAAUUUACACAA | OnXSSSSSSSSSSSSSSS |
| 36648 | SfA*SfC*SfA*SfA*SmA*SmG*SmG*SmC*SmAn001mA*SmU*SmG*SC* | AGGCAAUGCCAUCAC | SSSnXSSSSOnXSSnX |
| | SCsm04An001mU*SmC*SmAn001mC | | |
| WV- | Mod001L001fAn001fC*SfA*SfU*SfA*SfA*SfU*SfU*SfU*SfA*SfC* | ACAUAAUUUACACAA | OnXSSSSSSSSSSSSSSS |
| 36650 | SfA*SfC*SfA*SfA*SmA*SmG*SmG*SmC*SmAn001mA*SmU*SmG*SC* | AGGCAAUGCCAUCAC | SSSnXSSSSnXnXSSnX |
| | SCsm04n001An001mU*SmC*SmAn001mC | | |
| WV- | Mod001L001fAn001fC*SfA*SfU*SfAn001fA*SfU*SfU*SfU*SfA*SfC* | ACAUAAUUUACACAA | OnXSSSnXSSSSSSSSnX |
| 36651 | SfA*SfC*SfAn001fA*SmA*SmG*SmG*SmC*SmAn001mA*SmU*SmG*SC* | AGGCAAUGCCAUCAC | SSSSSnXSSSSOnXSSnX |
| | SCsm04An00ImU*SmC*SmAn001mC | | |
| WV- | Mod001L001fAn001fC*SfA*SfU*SfAn001fA*SfU*SfU*SfU*SfA*SfC* | ACAUAAUUUACACAA | OnXSSSnXSSSSSSSSnX |
| 36652 | SfA*SfC*SfAn001fA*SmA*SmG*SmG*SmC*SmAn001mA*SmU*SmG*SC* | AGGCAAUGCCAUCAC | SSSSSnXSSSSXnXSSnX |
| | SCsm04*An001mU*SmC*SmAn001mC | | |
| WV- | Mod001L001fAn001fC*SfA*SfU*SfA*SfA*SfU*SfU*SfU*SfA*SfC* | ACAUAAUUUACACAA | OnXSSSSSSSSSSSSSSS |
| 36653 | SfA*SfC*SfA*SfA*SmA*SmG*SmG*SmC*SmAn001mA*SmU*SmG*SC* | GAGCAAUGCUAUCAC | SSSnXSSSSOnXSSnX |
| | SUsm04An001mU*SmC*SmAn001mC | | |
| WV- | Mod001L001fAn001fC*SfA*SfU*SfA*SfA*SfU*SfU*SfU*SfA*SfC* | ACAUAAUUUACACAA | OnXSSSSSSSSSSSSSSS |
| 36655 | SfA*SfC*SfA*SfA*SmA*SmG*SmG*SmC*SmAn001mA*SmU*SmG*SC* | GAGCAAUGCUAUCAC | SSSnXSSSSnXnXSSnX |
| | SUsm04n001An001mU*SmC*SmAn001mC | | |
| WV- | Mod001L001fAn001fC*SfA*SfU*SfAn001fA*SfU*SfU*SfU*SfA* | ACAUAAUUUACACAA | OnXSSSnXSSSSSSSSnX |
| 36657 | SfC*SfA*SfC*SfAn001fA*SmA*SmG*SmG*SmC*SmAn001mA*SmU*SmG* | AGGCAAUGCUAUCAC | SSSSSnXSSSSXnXSSnX |
| | SC*SUsm04*An001mU*SmC*SmAn001mC | | |

TABLE A2
Example oligonucleotides and/or compositions that target DMD.

| | | | Stereochemistry/ |
|---|---|---|---|
| ID | Description | Base Sequence | Linkage |
| WV- | fU*SfC*SfA*SfC*SfU*SfC*SmAfG*SfA*SmU*SfA*SmGmUfU*SfG*SfA*SfA* | UCACUCAGAUAGUUGA | SSSSSSOSSSSOOSSSSS |
| 10258 | SfG*SfC*SfC | AGCC | S |
| WV- | fU*SfC*SfA*SfC*SfU*SfC*SmAn001fG*SfA*SmU*SfA*SmGn001mUn001fU* | UCACUCAGAUAGUUGA | SSSSSSnXSSSSnXnXSS |
| 11345 | SfG*SfA*SfA*SfG*SfC*SfC | AGCC | SSSS |
| WV- | fU*SfC*SfAn001fC*SfU*SfCn001mAfG*SfA*SmU*SfA*SmGmUfU*SfG*SfA* | UCACUCAGAUAGUUGA | SSnXSSnXOSSSSOOSSS |
| 12885 | SfAn001fG*SfC*SfC | AGCC | nXSS |
| WV- | fU*SfC*SfAn001RfC*SfU*SfCn001RmAfG*SfA*SmU*SfA*SmGmU*SfU*SfG* | UCACUCAGAUAGUUGA | SSnRSSnROSSSSOSSSS |
| 21218 | SfA*SfAn001RfG*SfC*SfC | AGCC | nRSS |
| WV- | L009n001L009n001L009n001L009fU*SfC*SfA*SfC*SfU*SfC*SmAfG*SfA* | UCACUCAGAUAGUUGA | nXnXnXOSSSSSSOSSSS |
| 23576 | SmU*SfA*SmGmUfU*SfG*SfA*SfA*SfG*SfC*SfC | AGCC | OOSSSSSS |
| WV- | L009n001L009n001L009n001fU*SfC*SfA*SfC*SfU*SfC*SmAfG*SfA*SmU* | UCACUCAGAUAGUUGA | nXnXnXSSSSSSOSSSSO |
| 23577 | SfA*SmGmUfU*SfG*SfA*SfA*SfG*SfC*SfC | AGCC | OSSSSSS |
| WV- | L009n001L009n001L009n001L009fU*SfC*SfAn001fC*SfU*SfCn001mAfG* | UCACUCAGAUAGUUGA | nXnXnXOSSnXSSnXOSS |
| 23578 | SfA*SmU*SfA*SmGmUfU*SfG*SfA*SfAn001fG*SfC*SfC | AGCC | SSOOSSSnXSS |
| WV- | L009n001L009n001L009n001fU*SfC*SfAn001fC*SfU*SfCn001mAfG*SfA* | UCACUCAGAUAGUUGA | nXnXnXSSnXSSnXOSSS |
| 23579 | SmU*SfA*SmGmUfU*SfG*SfA*SfAn001fG*SfC*SfC | AGCC | SOOSSSnXSS |
| WV- | L010n001L010n001L010n001L009fU*SfC*SfA*SfC*SfU*SfC*SmAfG*SfA* | UCACUCAGAUAGUUGA | nXnXnXOSSSSSSOSSSS |
| 23936 | SmU*SfA*SmGmUfU*SfG*SfA*SfA*SfG*SfC*SfC | AGCC | OOSSSSSS |
| WV- | L010n001L010n001L010n001fU*SfC*SfA*SfC*SfU*SfC*SmAfG*SfA*SmU* | UCACUCAGAUAGUUGA | nXnXnXSSSSSSOSSSSO |
| 23937 | SfA*SmGmUfU*SfG*SfA*SfA*SfG*SfC*SfC | AGCC | OSSSSSS |
| WV- | L010n001L010n001L010n001L009fU*SfC*SfAn001fC*SfU*SfCn001mAfG* | UCACUCAGAUAGUUGA | nXnXnXOSSnXSSnXOSS |
| 23938 | SfA*SmU*SfA*SmGmUfU*SfG*SfA*SfAn001fG*SfC*SfC | AGCC | SSOOSSSnXSS |
| WV- | L010n001L010n001L010n001fU*SfC*SfAn001fC*SfU*SfCn001mAfG*SfA* | UCACUCAGAUAGUUGA | nXnXnXSSnXSSnXOSSS |
| 23939 | SmU*SfA*SmGmUfU*SfG*SfA*SfAn001fG*SfC*SfC | AGCC | SOOSSSnXSS |
| WV- | fU*SfC*SfA*SfC*SfU*SfC*SmAn006RfG*SfA*SmU*SfA*SmGn006RmUn006R | UCACUCAGAUAGUUGA | SSSSSSnRSSSSnRnRSS |
| 24092 | fU*SfG*SfA*SfA*SfG*SfC*SfC | AGCC | SSSS |
| WV- | fU*SfC*SfA*SfC*SfU*SfC*SmAn008RfG*SfA*SmU*SfA*SmGn008RmUn008R | UCACUCAGAUAGUUGA | SSSSSSnRSSSSnRnRSS |
| 24098 | fU*SfG*SfA*SfA*SfG*SfC*SfC | AGCC | SSSS |
| WV- | 1T*SfC*S1A*SfC*SfU*SfC*SmAfG*SfA*SmU*SfA*SmGmUfU*SfG*SfA*SfA* | TCACUCAGAUAGUUGA | SSSSSSOSSSSOOSSSSS |
| 25538 | S1G*SfC*SfC | AGCC | S |
| WV- | fU*SfC*SfA*SfC*SfU*SfC*S1AfG*SfA*SmU*SfA*S1G1T1T*SfG*SfA*SfA* | UCACUCAGAUAGTTGA | SSSSSSOSSSSOOSSSSS |
| 25540 | SfG*SfC*SfC | AGCC | S |
| WV- | fU*SfC*SfA*SfC*SfU*SfC*S1An001RfG*SfA*SmU*SfA*S1Gn001R1Tn001R | UCACUCAGAUAGTTGA | SSSSSSnRSSSSnRnRSS |
| 25541 | 1T*SfG*SfA*SfA*SfG*SfC*SfC | AGCC | SSSS |
| WV- | 1T*SfC*S1A*SfC*SfU*SfC*SmAn001RfG*SfA*SmU*SfA*SmGn001RmUn001R | TCACUCAGAUAGUUGA | SSSSSSnRSSSSnRnRSS |
| 25542 | fU*SfG*SfA*SfA*SfG*SfC*SfC | AGCC | SSSS |
| WV- | fU*SfC*SfA*SfC*SfU*SfC*SmAn001RfG*SfA*SmU*SfA*SmGn001RmUn001R | UCACUCAGAUAGUUGA | SSSSSSnRSSSSnRnRSS |
| 25543 | fU*SfG*SfA*SfA*S1G*SfC*SfC | AGCC | SSSS |
| WV- | 1T*SfC*S1A*SfC*SfU*SfC*SmAn001RfG*SfA*SmU*SfA*SmGn001RmUn001R | TCACUCAGAUAGUUGA | SSSSSSnRSSSSnRnRSS |
| 25544 | fU*SfG*SfA*SfA*S1G*SfC*SfC | AGCC | SSSS |
| WV- | fU*SfC*SAsm01n001fC*SfU*SCsm01n001mAfG*SfA*SmU*SfA*SmGmU*SfU* | UCACUCAGAUAGUUGA | SSnXSSnXOSSSSOSSSS |
| 28294 | SfG*SfA*SAsm01n001fG*SfC*SfC | AGCC | nXSS |
| WV- | fU*SfC*SfA*SfC*SfU*SfC*SAsm01n001fG*SfA*SmU*SfA*SGsm01n001Tsm | UCACUCAGAUAGTUGA | SSSSSSnXSSSSnXnXSS |
| 28471 | 01n001fU*SfG*SfA*SfA*SfG*SfC*SfC | AGCC | SSSS |
| WV- | fU*SfC*SfA*SfC*SfU*SfC*SmAfG*SfA*SmU*SfA*SmGmUTsm01n001Gsm01n | UCACUCAGAUAGUTGA | SSSSSSOSSSSOOnXnXn |
| 28472 | 001Asm01n001Asm01n001Gsm01n001Csm01n001fC | AGCC | XnXnXnX |
| WV- | Tsm01n001Csm01n001Asm01n001Csm01n001Tsm01n001Csm01n001mAGsm01 | TCACTCAGATAGUTGA | nXnXnXnXnXnXOnXnXn |
| 28475 | n001Asm01n001Tsm01n001Asm01n001mGmUTsm01n001Gsm01n001Asm01n | AGCC | XnXOOnXnXnXnXnXnX |
| | 001Asm01n001Gsm01n001Csm01n001fC | | |
| WV- | fU*SfC*SfA*SfC*SfU*SfC*SmAGsm01n001Asm01n001Tsm01n001Asm01n | UCACUCAGATAGUUGA | SSSSSSOnXnXnXnXOOS |
| 28476 | 001mGmUfU*SfG*SfA*SfA*SfG*SfC*SfC | AGCC | SSSSS |
| WV- | Tsm01n001Csm01n001Asm01n001Csm01n001Tsm01n001Csm01n001mAfG* | TCACTCAGAUAGUUGA | nXnXnXnXnXnXOSSSSO |
| 28477 | SfA*SmU*SfA*SmGmUfU*SfG*SfA*SfA*SfG*SfC*SfC | AGCC | OSSSSSS |
| WV- | L023L009n009L009n009L009n009fU*SfC*SfA*SfC*SfU*SfC*SmAfG*SfA* | UCACUCAGAUAGUUGA | OnXnXnXSSSSSSOSSSS |
| 28763 | SmU*SfA*SmGmUfU*SfG*SfA*SfA*SfG*SfC*SfC | AGCC | OOSSSSSS |
| WV- | L023L010n009L010n009L010n009fU*SfC*SfA*SfC*SfU*SfC*SmAfG*SfA* | UCACUCAGAUAGUUGA | OnXnXnXSSSSSSOSSSS |
| 28764 | SmU*SfA*SmGmUfU*SfG*SfA*SfA*SfG*SfC*SfC | AGCC | OOSSSSSS |
| WV- | L023L018n009L018n009L018n009fU*SfC*SfA*SfC*SfU*SfC*SmAfG*SfA* | UCACUCAGAUAGUUGA | OnXnXnXSSSSSSOSSSS |
| 28765 | SmU*SfA*SmGmUfU*SfG*SfA*SfA*SfG*SfC*SfC | AGCC | OOSSSSSS |
| WV- | L023L019n009L019n009L019n009fU*SfC*SfA*SfC*SfU*SfC*SmAfG*SfA* | UCACUCAGAUAGUUGA | OnXnXnXSSSSSSOSSSS |
| 28766 | SmU*SfA*SmGmUfU*SfG*SfA*SfA*SfG*SfC*SfC | AGCC | OOSSSSSS |
| WV- | L023L016n001L016n001L016n001fU*SfC*SfA*SfC*SfU*SfC*SmAfG*SfA* | UCACUCAGAUAGUUGA | OnXnXnXSSSSSSOSSSS |
| 28767 | SmU*SfA*SmGmUfU*SfG*SfA*SfA*SfG*SfC*SfC | AGCC | OOSSSSSS |
| WV- | L023L017n001L017n001L017n001fU*SfC*SfA*SfC*SfU*SfC*SmAfG*SfA* | UCACUCAGAUAGUUGA | OnXnXnXSSSSSSOSSSS |
| 28768 | SmU*SfA*SmGmUfU*SfG*SfA*SfA*SfG*SfC*SfC | AGCC | OOSSSSSS |
| WV- | fU*SfC*SfAn009fC*SfU*SfCn009mAfG*SfA*SmU*SfA*SmGmUfU*SfG*SfA* | UCACUCAGAUAGUUGA | SSnXSSnXOSSSSOOSSS |
| 28769 | SfAn009fG*SfC*SfC | AGCC | nXSS |
| WV- | fU*SfC*SfAn009RfC*SfU*SfCn009RmAfG*SfA*SmU*SfA*SmGmU*SfU*SfG* | UCACUCAGAUAGUUGA | SSnRSSnROSSSSOSSSS |
| 28770 | SfA*SfAn009RfG*SfC*SfC | AGCC | nRSS |
| WV- | fU*SfC*SfA*SfC*SfU*SfC*SmAn009fG*SfA*SmU*SfA*SmGn009mUn009fU* | UCACUCAGAUAGUUGA | SSSSSSnXSSSSnXnXSS |
| 28771 | SfG*SfA*SfA*SfG*SfC*SfC | AGCC | SSSS |
| WV- | L023L009n001L009n001L009n001fU*SfC*SfA*SfC*SfU*SfC*SmAfG*SfA* | UCACUCAGAUAGUUGA | OnXnXnXSSSSSSOSSSS |
| 28800 | SmU*SfA*SmGmUfU*SfG*SfA*SfA*SfG*SfC*SfC | AGCC | OOSSSSSS |
| WV- | L023L010n001L010n001L010n001fU*SfC*SfA*SfC*SfU*SfC*SmAfG*SfA* | UCACUCAGAUAGUUGA | OnXnXnXSSSSSSOSSSS |
| 28801 | SmU*SfA*SmGmUfU*SfG*SfA*SfA*SfG*SfC*SfC | AGCC | OOSSSSSS |
| WV- | L023L010n001L010n001L010n001L009fU*SfC*SfA*SfC*SfU*SfC*SmAfG* | UCACUCAGAUAGUUGA | OnXnXnXOSSSSSSOSSS |
| 31610 | SfA*SmU*SfA*SmGmUfU*SfG*SfA*SfA*SfG*SfC*SfC | AGCC | SOOSSSSSS |
| WV- | fU*SfC*SfAn001RfC*SfU*Sfn001RmAfG*SfA*SmU*SfA*SmGmU*SfU*SfG* | UCACUCAGAUAGUUGA | SSnRSSnROSSSSOSSSS |
| 21218 | SfA*SfAn001RfG*SfC*SfC | AGCC | nRSS |
| WV- | fU*SfC*SfA*SfC*SfU*SfC*SmAfG*S1A*SmU*SfA*SmGmU*SfU*SFG*SfA* | UCACUCAGAUAGUUGA | SSSSSSOSSSSOSSSSSS |
| 22749 | SfA*SfG*SfC*SfC | AGCC | S |
| WV- | fU*SFC*SfAn02SR1C*SfU*SfCn02SRmA1G*SfA*SmU*SA*SmGmU*SfU*Sf* | UCACUCAGAUAGUUGA | SSnRSSnROSSSSOSSSS |
| 43239 | SfA*SfAn025RfG*SfC*SfC | AGCC | nRSS |
| WV- | fU*SfC*SAn026RC*SfU*SfCn026RmAfG*SfA*SmU*SfA*SmGmU*SfU*SfG* | UCACUCAGAUAGUUGA | SSnRSSnROSSSSOSSSS |
| 43240 | SfA*SfAn026RfG*Sf*SfC | AGCC | nRSS |
| WV- | fU*SfC*SfAn030RfC*SfU*SfC0030RmAfG*SfA*SmU*SfA*SmGmU*SfU*SfG* | UCACUCAGAUAGUUGA | SSnRSSnROSSSSOSSSS |
| 43241 | SfA*SfAn030RfG*SfC*SfC | AGCC | nRSS |
| WV- | fU*SfC*SfAn031RfC*SfU*SfCn031RmAfG*SfA*SmU*SfA*SmGmU*SfU*SfG* | UCACUCAGAUAGUUGA | SSnRSSnROSSSSOSSSS |
| 43242 | SfA*SfAn031RfG*SfC*SfC | AGCC | nRSS |
| WV- | fU*SfC*SfAn036RfC*SfU*SfCn036RmAfG*SfA*SmU*SfA*SmGmU*SfU*SfG* | UCACUCAGAUAGUUGA | SSnRSSnROSSSSOSSSS |
| 43243 | SfA*SfAn036RfG*SfC*SfC | AGCC | nRSS |
| WV- | fU*SfC*SfAn037RfC*SfU*SfCn037RmAfG*SfA*SmU*SfA*SmGmU*SfU*SfG* | UCACUCAGAUAGUUGA | SSnRSSnROSSSSOSSSS |
| 43244 | SfA*SfAn037RfG*SfC*SfC | AGCC | nRSS |
| WV- | fU*SfC*SfAn004RfC*SfU*SCn004RmAfG*SfA*SmU*SfA*SmGmU*SfU*SfG* | UCACUCAGAUAGUUGA | SSnRSSnROSSSSOSSSS |
| 43287 | SfA*SfAn004RfG*SfC*SfC | AGCC | nRSS |
| WV- | fU*C*A*C*fU*C*mAfG*fA*mU*A*mGmU*U*fG*fA*fA*SG*EC*C | UCACUCAGAUAGUUGA | XXXXXXOXXXXOXXXXXX |
| 32062 | | AGCC | X |
| WV- | 11*RfC*01A*RfC*RfU*R1C*RmAfG*R1A*RmU*R1A*RmGmU*RfU*RfG*RfA* | UCACUCAGAUAGUUGA | RRRRRRORRRRORRRRRR |
| 34789 | RfA*RfG*RfU*RfC | AGCC | R |
| WV- | fU*SfC*SfA*SfC*SfU*SfC*SmAfG*SfA*SmU*SfA*SmGmU*SfU*SfG*SfA* | UCACUCAGAUAGUUGA | SSSSSSOSSSSOSSSSSS |
| 22749 | SfA*SfG*SfC*SfC | AGCC | S |
| WV- | fU*fC*fAn001fC*fU*fCn001mAfG*fA*mU*fA*mGmU*fU*fG*fA*fAn001fGU | UCACUCAGAUAGUUGA | XXnXXXnXOXXXXOXXXX |
| 32063 | U*1f | AGCC | nXXX |
| WV- | fU*RfC*RfAn001fC*RfU*RfCm001mAfG*RfA*RmU*RfA*RmGmU*RfU*RfG* | UCACUCAGAUAGUUGA | RRnXRRnXORRRRORRRR |
| 34790 | RtA*RfAn001fG*RfC*RfC | AGCC | nXRR |
| WV- | fU*SfC*SfAn001fC*SfU*SfCn001mAfG*SfA*SmU*SfA*SmGmU*SfU*SfG* | UCACUCAGAUAGUUGA | SSnXSSnXOSSSSOSSSS |
| 31377 | SfA*SfAn001fG*SfC*SfC | AGCC | nXSS |
| WV- | fU*SfC*SfAn001R1C*SfU*SfCn001RmAfG*SfA*SmU*SfA*SmGmU*SfU*SfG* | UCACUCAGAUAGUUGA | SSnRSSnROSSSSOSSSS |
| 21218 | SfA*SfAn001RfG*SfC*SfC | AGCC | nRSS |
| WV- | fU*SfC*SfAn001SfC*SfU*SfCn001SmAfG*SfA*SmU*SfA*SmGmU*SfU*SfG* | UCACUCAGAUAGUUGA | SSnSSSnSOSSSSOSSSS |
| 31386 | SfA*S1An001SfG*SfC*SfU | AGCC | nSSS |

TABLE A3
Example oligonucleotides and/or compositions that target MALATI.

| | | | Stereochemistry/ |
|---|---|---|---|
| ID | Description | Base Sequence | Linkage |
| WV- | mU*SGeom5Ceom5CeomA*SG*SG*SC*ST*SG*SG*RT*ST*SA*ST*SmG* | UGCCAGGCTGGTTATG | SOOOSSSSSSRSSSSSSS |
| 8584 | SmA*SmC*SmU*SmC | ACUC | S |
| WV- | mU*SGeom5Ceom5CeomA*SG*SG*RC*ST*SG*RG*ST*ST*RA*ST*SmG* | UGCCAGGCTGGTTATG | SOOOSSRSSRSSRSSSSS |
| 8587 | SmA*SmC*SmU*SmC | ACUC | S |
| WV- | mU*SGeon001m5Ceon001m5Ceon001mA*SG*SG*RC*ST*SG*RG*ST*ST* | UGCCAGGCTGGTTATG | SnXnXnXSSRSSRSSRSS |
| 11533 | RA*ST*SmG*SmA*SmC*SmU*SmC | ACUC | SSSS |
| WV- | Mod001L001mU*SGeom5Ceom5CeomA*SG*SG*RC*ST*SG*RG*ST*ST* | UGCCAGGCTGGTTATG | OSOOOSSRSSRSSRSSSS |
| 12503 | RA*ST*SmG*SmA*SmC*SmU*SmC | ACUC | SS |
| WV- | Mod001L001mU*SGeon001m5Ceon001m5Ceon001mA*SG*SG*RC*ST* | UGCCAGGCTGGTTATG | OSnXnXnXSSRSSRSSRS |
| 12504 | SG*RG*ST*ST*RA*ST*SmG*SmA*SmC*SmU*SmC | ACUC | SSSSS |
| WV- | mU*SGeom5Ceom5CeomA*SG*SG*RC*ST*SG*RG*ST*ST*RA*ST*SmGn | UGCCAGGCTGGTTATG | SOOOSSRSSRSSRSSnXn |
| 13303 | 001mAn001mCn001mU*SmC | ACUC | XnXS |
| WV- | mU*SGeon001m5Ceon001m5Ceon001mA*SG*SG*RC*ST*SG*RG*ST*ST* | UGCCAGGCTGGTTATG | SnXnXnXSSRSSRSSRSS |
| 13304 | RA*ST*SmGn001mAn001mCn001mU*SmC | ACUC | nXnXnXS |
| WV- | mU*SGeon006Rm5Ceon006Rm5Ceon006RmA*SG*SG*RC*ST*SG*RG*ST* | UGCCAGGCTGGTTATG | SnRnRnRSSRSSRSSRSS |
| 24104 | ST*RA*ST*SmG*SmA*SmC*SmU*SmC | ACUC | SSSS |
| WV- | mU*SGeon008Rm5Ceon008Rm5Ceon008RmA*SG*SG*RC*ST*SG*RG*ST* | UGCCAGGCTGGTTATG | SnRnRnRSSRSSRSSRSS |
| 24109 | ST*RA*ST*SmG*SmA*SmC*SmU*SmC | ACUC | SSSS |
| WV- | mU*SGsm01n001Csm01n001Csm01n001mA*SG*SG*RC*ST*SG*RG*ST* | UGCCAGGCTGGTTATG | SnXnXnXSSRSSRSSRSS |
| 28299 | ST*RA*ST*SmG*SmA*SmC*SmU*SmC | ACUC | SSSS |
| WV- | mU*SGsm01n001Csm01n001Csm01n001mA*SG*SG*RC*ST*SG*RG*ST* | UGCCAGGCTGGTTATG | SnXnXnXSSRSSRSSRSS |
| 28468 | ST*RA*ST*SmG*SAsm01n001Csm01n001Tsm01n001mC | ACTC | SnXnXnX |
| WV- | mU*SGeom5Ceom5CeomA*SG*SG*RC*ST*SG*RG*ST*ST*RA*ST*SmG* | UGCCAGGCTGGTTATG | SOOOSSRSSRSSRSSSnX |
| 28469 | SAsm01n001Csm01n001Tsm01n001mC | ACTC | nXnX |
| WV- | mU*SGeon001m5Ceon001m5Ceon001mA*SG*SG*RC*ST*SG*RG*ST*ST* | UGCCAGGCTGGTTATG | SnXnXnXSSRSSRSSRSS |
| 28470 | RA*ST*SmG*SAsm01n001Csm01n001Tsm01n001mC | ACTC | SnXnXnX |
| WV- | mU*SGsm01n001m5CeoCsm01n001mA*SG*SG*RC*ST*SG*RG*ST*ST* | UGCCAGGCTGGTTATG | SnXOnXSSRSSRSSRSSS |
| 31055 | RA*ST*SmG*SmA*SmC*SmU*SmC | ACUC | SSS |
| WV- | mU*SGsm01n001Csm01n001m5CeomA*SG*SG*RC*ST*SG*RG*ST*ST* | UGCCAGGCTGGTTATG | SnXnXOSSRSSRSSRSSS |
| 31056 | RA*ST*SmG*SmA*SmC*SmU*SmC | ACUC | SSS |
| WV- | mU*SGeoCsm01n001Csm01n001mA*SG*SG*RC*ST*SG*RG*ST*ST*RA* | UGCCAGGCTGGTTATG | SOnXnXSSRSSRSSRSSS |
| 31057 | ST*SmG*SmA*SmC*SmU*SmC | ACUC | SSS |
| WV- | Tsm01n001Gsm01n001Csm01n001Csm01n001mA*SG*SG*RC*ST*SG* | TGCCAGGCTGGTTATG | nXnXnXnXSSRSSRSSRS |
| 31058 | RG*ST*ST*RA*ST*SmG*SmA*SmC*SmU*SmC | ACUC | SSSSS |
| WV- | Tsm01n001Gsm01n001Csm01n001m5CeomA*SG*SG*RC*ST*SG*RG*ST* | TGCCAGGCTGGTTATG | nXnXnXOSSRSSRSSRSS |
| 31059 | ST*RA*ST*SmG*SmA*SmC*SmU*SmC | ACUC | SSSS |
| WV- | Tsm01n001Gsm01n001m5CeoCsm01n001mA*SG*SG*RC*ST*SG*RG*ST* | TGCCAGGCTGGTTATG | nXnXOnXSSRSSRSSRSS |
| 31060 | ST*RA*ST*SmG*SmA*SmC*SmU*SmC | ACUC | SSSS |
| WV- | Tsm01n001GeoCsm01n001Csm01n001mA*SG*SG*RC*ST*SG*RG*ST* | TGCCAGGCTGGTTATG | nXOnXnXSSRSSRSSRSS |
| 31061 | ST*RA*ST*SmG*SmA*SmC*SmU*SmC | ACUC | SSSS |
| WV- | Tsm01n001Geom5CeoCsm01n001mA*SG*SG*RC*ST*SG*RG*ST*ST*RA* | TGCCAGGCTGGTTATG | nXOOnXSSRSSRSSRSSS |
| 31062 | ST*SmG*SmA*SmC*SmU*SmC | ACUC | SSS |
| WV- | Tsm01n001Geom5CeoCsm01n001mA*SG*SG*RC*ST*SG*RG*ST*ST*RA* | TGCCAGGCTGGTTATG | nXOOnXSSRSSRSSRSSS |
| 31063 | ST*SmG*SAsm01n001mC*SmU*SmC | ACUC | nXSS |
| WV- | mU*SGsm01n001m5CeoCsm01n001mA*SG*SG*RC*ST*SG*RG*ST*ST* | UGCCAGGCTGGTTATG | SnXOnXSSRSSRSSRSSS |
| 31064 | RA*ST*SmG*SAsm01n001mC*SmU*SmC | ACUC | nXSS |
| WV- | mU*SGeon001Csm01n001m5Ceon001mA*SG*SG*RC*ST*SG*RG*ST*ST* | UGCCAGGCTGGTTATG | SnXnXnXSSRSSRSSRSS |
| 31065 | RA*ST*SmG*SmA*SmC*SmU*SmC | ACUC | SSSS |
| WV- | mU*SGsm01n001m5Ceon001Csm01n001mA*SG*SG*RC*ST*SG*RG*ST* | UGCCAGGCTGGTTATG | SnXnXnXSSRSSRSSRSS |
| 31066 | ST*RA*ST*SmG*SmA*SmC*SmU*SmC | ACUC | SSSS |
| WV- | mU*SGeon001m5Ceon001m5Ceon001mA*SG*SGsm01n001C*ST*SG*RG* | UGCCAGGCTGGTTATG | SnXnXnXSSnXSSRSSRS |
| 31067 | ST*ST*RA*ST*SmG*SmA*SmC*SmU*SmC | ACUC | SSSSS |
| WV- | mU*SGeon001m5Ceon001m5Ceon001mA*SG*SG*RC*ST*SGsm01n001G* | UGCCAGGCTGGTTATG | SnXnXnXSSRSSnXSSRS |
| 31068 | ST*ST*RA*ST*SmG*SmA*SmC*SmU*SmC | ACUC | SSSSS |
| WV- | mU*SGeon001m5Ceon001m5Ceon001mA*SG*SG*RC*ST*SG*RG*ST*ST* | UGCCAGGCTGGTTATG | SnXnXnXSSRSSRSSRSn |
| 31069 | RA*STsm01n001mG*SmA*SmC*SmU*SmC | ACUC | XSSSS |
| WV- | mU*SGsm01n001m5CeoCsm01n001mA*SG*SG*RC*ST*SG*RG*ST*ST* | UGCCAGGCTGGTTATG | SnXOnXSSRSSRSSRSnX |
| 31070 | RA*STsm01n001mG*SmA*SmC*SmU*SmC | ACUC | SSSS |
| WV- | mU*SGsm01n001m5CeoCsm01n001mA*SG*SGsm01n001C*ST*SG*RG*ST | UGCCAGGCTGGTTATG | SnXOnXSSnXSSRSSRSS |
| 31071 | *ST*RA*ST*SmG*SmA*SmC*SmU*SmC | ACUC | SSSS |
| WV- | mU*SGsm01n001m5CeoCsm01n001mA*SG*SG*RC*ST*SGsm01n001G*ST | UGCCAGGCTGGTTATG | SnXOnXSSRSSnXSSRSS |
| 31072 | *ST*RA*ST*SmG*SmA*SmC*SmU*SmC | ACUC | SSSS |
| WV- | mU*SGsm01n001m5CeoCsm01n001mA*SG*SG*RC*ST*SG*RG*ST*STsm | UGCCAGGCTGGTTATG | SnXOnXSSRSSRSSnXSS |
| 31073 | 01n001A*ST*SmG*SmA*SmC*SmU*SmC | ACUC | SSSS |
| WV- | mU*SGsm01n001m5CeoCsm01n001mA*SG*SG*RC*ST*SG*RG*ST*STsm | UGCCAGGCTGGTTATG | SnXOnXSSRSSRSSnXSS |
| 31073 | 01n001A*ST*SmG*SmA*SmC*SmU*SmC | ACUC | SSSS |
| WV- | Teo*SGsm01n001m5CeoCsm01n001Aeo*SG*SG*RC*ST*SG*RG*ST*ST* | TGCCAGGCTGGTTATG | SnXOnXSSRSSRSSRSSS |
| 31095 | RA*ST*SmG*SmA*SmC*SmU*SmC | ACUC | SSS |
| WV- | Teo*SGsm01n001Csm01n001m5CeoAeo*SG*SG*RC*ST*SG*RG*ST*ST* | TGCCAGGCTGGTTATG | SnXnXOSSRSSRSSRSSS |
| 31096 | RA*ST*SmG*SmA*SmC*SmU*SmC | ACUC | SSS |
| WV- | Teo*SGeoCsm01n001Csm01n001Aeo*SG*SG*RC*ST*SG*RG*ST*ST*RA | TGCCAGGCTGGTTATG | SOnXnXSSRSSRSSRSSS |
| 31097 | *ST*SmG*SmA*SmC*SmU*SmC | ACUC | SSS |
| WV- | Teo*SGeon001Csm01n001m5Ceon001Aeo*SG*SG*RC*ST*SG*RG*ST* | TGCCAGGCTGGTTATG | SnXnXnXSSRSSRSSRSS |
| 31098 | ST*RA*ST*SmG*SmA*SmC*SmU*SmC | ACUC | SSSS |
| WV- | Teo*SGsm01n001m5Ceon001Csm01n001Aeo*SG*SG*RC*ST*SG*RG*ST | TGCCAGGCTGGTTATG | SnXnXnXSSRSSRSSRSS |
| 31099 | *ST*RA*ST*SmG*SmA*SmC*SmU*SmC | ACUC | SSSS |
| WV- | Teo*SGeon001m5Ceon001m5Ceon001Aeo*SG*SGsm01n001C*ST*SG* | TGCCAGGCTGGTTATG | SnXnXnXSSnXSSRSSRS |
| 31100 | RG*ST*ST*RA*ST*SmG*SmA*SmC*SmU*SmC | ACUC | SSSSS |
| WV- | Teo*SGeon001m5Ceon001m5Ceon001Aeo*SG*SG*RC*ST*SGsm01n001 | TGCCAGGCTGGTTATG | SnXnXnXSSRSSnXSSRS |
| 31101 | G*ST*ST*RA*ST*SmG*SmA*SmC*SmU*SmC | ACUC | SSSSS |
| WV- | Teo*SGeon001m5Ceon001m5Ceon001Aeo*SG*SG*RC*ST*SG*RG*ST* | TGCCAGGCTGGTTATG | SnXnXnXSSRSSRSSRSn |
| 31102 | ST*RA*STsm01n001mG*SmA*SmC*SmU*SmC | ACUC | XSSSS |
| WV- | Teo*SGsm01n001m5CeoCsm01n001Aeo*SG*SG*RC*ST*SG*RG*ST*ST* | TGCCAGGCTGGTTATG | SnXOnXSSRSSRSSRSnX |
| 31103 | RA*STsm01n001mG*SmA*SmC*SmU*SmC | ACUC | SSSS |
| WV- | Teo*SGsm01n001m5CeoCsm01n001Aeo*SG*SGsm01n001C*ST*SG*RG* | TGCCAGGCTGGTTATG | SnXOnXSSnXSSRSSRSS |
| 31104 | ST*ST*RA*ST*SmG*SmA*SmC*SmU*SmC | ACUC | SSSS |
| WV- | Teo*SGsm01n001m5CeoCsm01n001Aeo*SG*SG*RC*ST*SGsm01n001G* | TGCCAGGCTGGTTATG | SnXOnXSSRSSnXSSRSS |
| 31105 | ST*ST*RA*ST*SmG*SmA*SmC*SmU*SmC | ACUC | SSSS |
| WV- | Teo*SGsm01n001m5CeoCsm01n001Aeo*SG*SG*RC*ST*SG*RG*ST*STs | TGCCAGGCTGGTTATG | SnXOnXSSRSSRSSnXSS |
| 31106 | m01n001A*ST*SmG*SmA*SmC*SmU*SmC | ACUC | SSSS |
| WV- | mU*SGsm01n001m5CeoCsm01n001mA*SG*SG*RC*ST*SG*RG*ST*ST*RA | UGCCAGGCTGGTTATG | SnXOnXSSRSSRSSRSSS |
| 31107 | *ST*SmG*SAeo*Sm5Ceo*STeo*SmC | ACTC | SSS |
| WV- | mU*SGsm01n001m5CeoCsm01n001mA*SG*SG*RC*ST*SG*RG*ST*ST*RA | UGCCAGGCTGGTTATG | SnXOnXSSRSSRSSRSSS |
| 31108 | *ST*SGeo*SAeo*Sm5Ceo*STeo*SmC | ACTC | SSS |
| WV- | U023U010n001U010n001U010n001L009mU*SGeom5Ceom5CeomA*SG* | UGCCAGGCTGGTTATG | OnXnXnXOSOOOSSRSSR |
| 31621 | SG*RC*ST*SG*RG*ST*ST*RA*ST*SmG*SmA*SmC*SmU*SmC | ACUC | SSRSSSSSS |
| WV- | U023U010n001U010n001U010n001mU*SGeom5Ceom5CeomA*SG*SG*RC | UGCCAGGCTGGTTATG | OnXnXnXSOOOSSRSSRS |
| 31622 | *ST*SG*RG*ST*ST*RA*ST*SmG*SmA*SmC*SmU*SmC | ACUC | SRSSSSSS |
| WV- | U023U010n009L009mU*SGeom5Ceom5CeomA*SG*SG*RC*ST*SG*RG*ST | UGCCAGGCTGGTTATG | OnXOSOOOSSRSSRSSRS |
| 32681 | *ST*RA*ST*SmG*SmA*SmC*SmU*SmC | ACUC | SSSSS |
| WV- | mU*SGeom5Ceom5CeomA*SG*SG*RC*ST*SG*RG*ST*ST*RA*ST*SmG*SA | UGCCAGGCTGGTTATG | SOOOSSRSSRSSRSSSOS |
| 37972 | sm01n013mC*SmU*SmC | ACUC | S |
| WV- | mU*SGeom5Ceom5CeoAsm01n013G*SG*RC*ST*SG*RG*ST*ST*RA*ST* | UGCCAGGCTGGTTATG | SOOOOSRSSRSSRSSSOS |
| 37973 | SmG*SAsm01n013mC*SmU*SmC | ACUC | S |
| WV- | mU*SGeom5Ceom5CeoAsm01n013G*SG*RC*ST*SG*RG*ST*ST*RA*ST* | UGCCAGGCTGGTTATG | SOOOOSRSSRSSRSSSSS |
| 37976 | SmG*SmA*SmC*SmU*SmC | ACUC | S |
| WV- | mU*SGeom5Ceom5CeoAsm01n013G*SG*RC*ST*SG*RG*ST*ST*RA*ST* | UGCCAGGCTGGTTATG | SOOOOSRSSRSSRSSSnR |
| 37980 | SmG*SmAn001RmC*SmU*SmC | ACUC | SS |
| WV- | mUn001RGeom5Ceom5Ceon001RAsm01n013G*SG*RC*ST*SG*RG*ST*ST | UGCCAGGCTGGTTATG | nROOnROSRSSRSSRSSS |
| 37981 | *RA*ST*SmG*SmA*SmC*SmU*SmC | ACUC | SSS |
| WV- | mUn001RGeom5Ceom5Ceon001RAsm01n013G*SG*RC*ST*SG*RG*ST*ST | UGCCAGGCTGGTTATG | nROOnROSRSSRSSRSSS |
| 37982 | *RA*ST*SmG*SmAn001RmC*SmU*SmC | ACUC | nRSS |
| WV- | mU*SGeon001Rm5Ceon001Rm5Ceon001RmA*SG*SG*RC*ST*SG*RG*ST* | UGCCAGGCTGGTTATG | SnRnRnRSSRSSRSSRSS |
| 15562 | ST*RA*ST*SmG*SmA*SmC*SmU*SmC | ACUC | SSSS |
| WV- | mU*SGeon002Rm5Ceon002Rm5Ceon002RmA*SG*SG*RC*ST*SG*RG*ST* | UGCCAGGCTGGTTATG | SnRnRnRSSRSSRSSRSS |
| 15885 | ST*RA*ST*SmG*SmA*SmC*SmU*SmC | ACUC | SSSS |
| WV- | mU*SGeon002Sm5Ceon002Sm5Ceon002SmA*SG*SG*RC*ST*SG*RG*ST* | UGCCAGGCTGGTTATG | SnSnSnSSSRSSRSSRSS |
| 15887 | ST*RA*ST*SmG*SmA*SmC*SmU*SmC | ACUC | SSSS |
| WV- | mU*SGcon006Rm5Ccon006Rm5Ceon006RmA*SG*SG*RC*ST*SG*RG*ST* | UGCCAGGCTGGTTATG | SnRnRnRSSRSSRSSRSS |
| 24104 | ST*RA*ST*SmG*SmA*SmC*SmU*SmC | ACUC | SSSS |
| WV- | mU*SGcon006Sm5Ceon006Sm5Ccon006SmA*SG*SG*RC*ST*SG*RG*ST* | UGCCAGGCTGGTTATG | SnSnSnSSSRSSRSSRSS |
| 24105 | ST*RA*ST*SmG*SmA*SmC*SmU*SmC | ACUC | SSSS |
| WV- | mU*SGsm01n001Csm01n001Csm01n001mA*SG*SG*RC*ST*SG*RG*ST* | UGCCAGGCTGGTTATG | SnXnXnXSSRSSRSSRSS |
| 28299 | ST*RA*ST*SmG*SmA*SmC*SmU*SmC | ACUC | SSSS |
| WV- | mUn001Geom5Ceo*Sm5Ceon001mA*SG*SG*RC*ST*SG*RG*ST*ST*RA* | UGCCAGGCTGGhaTGA | nXOSnXSSRSSRSSRSSS |
| 30938 | ST*SmG*SmAn001mC*SmU*SmC | CUC | nXSS |
| WV- | mU*SGeon001Rm5Ceom5Ceon001RmA*SG*SG*RC*ST*SG*RG*ST*ST* | UGCCAGGCTGGTTATG | SnROnRSSRSSRSSRSSS |
| 38642 | RA*ST*SmG*SmAn001RmC*SmU*SmC | ACUC | nRSS |
| WV- | mUsm09Geosm10m5Ceo*Sm5Ceon001mA*SG*SG*RC*ST*SG*RG*ST*ST* | UGCCAGGCTGGTTATG | OOSnXSSRSSRSSRSSSn |
| 39291 | RA*ST*SmG*SmAn001mC*SmU*SmC | ACUC | XSS |
| WV- | mUsm05Geosm06m5Ceo*SmSCeon001mA*SG*SG*RC*ST*SG*RG*ST*ST* | UGCCAGGCTGGTTATG | OOSnXSSRSSRSSRSSSn |
| 39402 | RA*ST*SmG*SmAn001mC*SmU*SmC | ACUC | XSS |
| WV- | mU*SGeo*n001m5Ceo*n001m5Ceo*n001mA*SG*SG*RC*ST*SG*RG*ST* | UGCCAGGCTGGTTATG | Sn*Xn*Xn*XSSRSSRSS |
| 40386 | ST*RA*ST*SmG*SmA*SmC*SmU*SmC | ACUC | RSSSSSS |
| WV- | mU*SGsm01*n001Csm01*n001Csm01*n001mA*SG*SG*RC*ST*SG*RG* | UGCCAGGCTGGTTATG | Sn*Xn*Xn*XSSRSSRSS |
| 40387 | ST*ST*RA*ST*SmG*SmA*SmC*SmU*SmC | ACUC | RSSSSSS |
| WV- | mUn001Geom5Ceom5CeomA*SG*SG*RC*ST*SG*RG*ST*ST*RA*ST*SmG* | UGCCAGGCTGGTTATG | nXOOOSSRSSRSSRSSSS |
| 40579 | SmA*SmC*SmU*SmC | ACUC | SS |
| WV- | mUsm05Geosm06m5Ceom5CeomA*SG*SG*RC*ST*SG*RG*ST*ST*RA*ST* | UGCCAGGCTGGTTATG | OOOOSSRSSRSSRSSSSS |
| 40807 | SmG*SmA*SmC*SmU*SmC | ACUC | S |
| WV- | mUsm09Gcosm10m5Ceom5CeomA*SG*SG*RC*ST*SG*RG*ST*ST*RA*ST* | UGCCAGGCTGGTTATG | OOOOSSRSSRSSRSSSSS |
| 40808 | SmG*SmA*SmC*SmU*SmC | ACUC | S |
| WV- | mUn001SGeom5Ceom5CeomA*SG*SG*RC*ST*SG*RG*ST*ST*RA*ST* | UGCCAGGCTGGTTATG | nSOOOSSRSSRSSRSSSS |
| 40831 | SmG*SmA*SmC*SmU*SmC | ACUC | SS |
| WV- | mUn001.RGeom5Ceom5CeomA*SG*SG*RC*ST*SG*RG*ST*ST*RA*ST* | UGCCAGGCTGGTTATG | nROOOSSRSSRSSRSSSS |
| 40832 | SmG*SmA*SmC*SmU*SmC | ACUC | SS |
| WV- | Tsm01n024Gcosm10m5Ceom5CeomA*SG*SG*RC*ST*SG*RG*ST*ST*RA* | TGCCAGGCTGGTTATG | OOOOSSRSSRSSRSSSSS |
| 40835 | ST*SmG*SmA*SmC*SmU*SmC | ACUC | S |
| WV- | mU*SGeon020Rm5Ceon020Rm5Ceon020RmA*SG*SG*RC*ST*SG*RG*ST* | UGCCAGGCTGGTTATG | SnRnRnRSSRSSRSSRSS |
| 41422 | ST*RA*ST*SmG*SmA*SmC*SmU*SmC | ACUC | SSSS |
| WV- | mU*SGeon020Sm5Ceon020SmSCeon020SmA*SG*SG*RC*ST*SG*RG*ST* | UGCCAGGCTGGTTATG | SnSnSnSSSRSSRSSRSS |
| 41430 | ST*RA*ST*SmG*SmA*SmC*SmU*SmC | ACUC | SSSS |
| WV- | mU*SGeon033Rm5Ceom5CeomA*SG*SG*RC*ST*SG*RG*ST*ST*RA*ST* | UGCCAGGCTGGTTATG | SnROOSSRSSRSSRSSSS |
| 43245 | SmG*SmA*SmC*SmU*SmC | ACUC | SS |
| WV- | mU*SGeon036Rm5CcomSCeomA*SG*SG*RC*ST*SG*RG*ST*ST*RA*ST* | UGCCAGGCTGGTTATG | SnROOSSRSSRSSRSSSS |
| 43246 | SmG*SmA*SmC*SmU*SmC | ACUC | SS |
| WV- | mU*SGcon037RmSCeom5CcomA*SG*SG*RC*STSG*RG*ST*ST*RA*ST* | UGCCAGGCTGGTTATG | SnROOSSRSSRSSRSSSS |
| 43247 | SmG*SmA*SmC*SmU*SmC | ACUC | SS |
| WV- | mU*SGeon004Rm5Ceon004Rm5Ceon004RmA*SG*SG*RC*ST*SG*RG*ST* | UGCCAGGCTGGTCATG | SnRnRnRSSRSSRSSRSS |
| 43248 | S1'*RA*ST*SmG*SmA*SmC*SmU*SmC | ACUC | SSSS |
| WV- | mU*SGcon025Rm5Ceon025Rm5Ceon025RmA*SG*SG*RC*ST*SG*RG*ST* | UGCCAGGCTGGTATGA | SnRnRnRSSRSSRSSRSS |
| 43249 | ST*RA*ST*SmG*SmA*SmC*SmU*SmC | CUC | SSSS |
| WV- | mU*SGeon026Rm5Ceon026Rm5Ceon026RmA*SG*SG*RC*ST*SG*RG*ST* | UGCCAGGCTGGTTATG | SnRnRnRSSRSSRSSRSS |
| 43250 | S1'*RA*ST*SmG*SmA*SmC*SmU*SmC | ACUC | SSSS |
| WV- | mU*SGeon030Rm5Ceon030Rm5Ceon030RmA*SG*SG*RC*ST*SG*RG*ST* | UGCCAGGCTGGTTATG | SnRnRnRSSRSSRSSRSS |
| 43251 | ST*RA*S1'*SroG*SmA*SmC*SmU*SmC | ACUC | SSSS |
| WV- | mU*SGeon031RmSCeon031Rm5Ceon031RmA*SG*SG*RC*ST*SG*RG*ST* | UGCCAGGCTGGTTATG | SnRnRnRSSRSSRSSRSS |
| 43252 | ST*RA*ST*SmG*SmA*SmC*SmU*SmC | ACUC | SSSS |
| WV- | mU*SGeon036Rm5Ceon036Rm5Ceon036RmA*SG*SG*RC*ST*SG*RG*ST* | UGCCAGGCTGGTTATG | SnRnRnRSSRSSRSSRSS |
| 43253 | ST*RA*ST*SroG*SmA*SmC*SmU*SmC | ACUC | SSSS |
| WV- | mU*SGeon037Rm5Ceon037Rm5Ceon037RmA*SG*SG*RC*ST*SG*RG*ST* | UGCCAGGCTGGTTATG | SnRnRnRSSRSSRSSRSS |
| 43254 | ST*RA*ST*SmG*SmA*SmC*SmU*SmC | ACUC | SSSS |
| WV- | mU*SGcon033Rm5Ccon001Rm5Ceon001RmA*SG*SG*RCST*SG*RG*ST* | UGCCAGGCTGGTTATG | SnRnRnRSSRSSRSSRSS |
| 43255 | ST*RA*ST*SmG*SmA*SmC*SmU*SmC | ACUC | SSSS |
| WV- | mU*SGeon003RmSCeon003RmSCeon003RmA*SG*SG*RC*ST*SG*RG*ST* | UGCCAGGCTGGTTATG | SnRnRnRSSRSSRSSRSS |
| 44357 | ST*RA*ST*SmG*SmA*SmC*SmU*SmC | ACUC | SSSS |
| WV- | mU*SGeo*n002m5Ceo*n002m5Ceo*n002mA*SG*SG*RC*ST*SG*RG*ST* | UGCCAGGC!GGTCATG | Sn*Xn*Xn*XSSRSSRSS |
| 44359 | ST*RA*ST*SmG*SmA*SmC*SmU*SmC | ACUC | RSSSSSS |
| WV- | mU*SGeo*n006m5Ceo*n006m5Ceo*n006mA*SG*SG*RC*ST*SG*RG*ST* | UGCCAGGCTGGTTATG | Sn*Xn*Xn*XSSRSSRSS |
| 44360 | ST*RA*ST*SmG*SmA*SmC*SmU*SroC | ACUC | RSSSSSS |
| WV- | mU*SGeo*n020m5Ceo*n020m5Ceo*n020mA*SG*SG*RC*ST*SG*RG*ST* | UGCCAGGCTGGTTATG | Sn*Xn*Xn*XSSRSSRSS |
| 44361 | ST*RA*ST*SmG*SmA*SmC*SmL*SmC | ACUC | RSSSSSS |
| WV- | mU*SGsm01n002Csm01n002Csm01n002mA*SG*SG*RC*ST*SG*RG*ST* | UGCCAGGCTGGTTATG | SnXnXnXSSRSSRSSRSS |
| 44364 | ST*RA*ST*SmG*SmA*SmC*SmU*SmC | ACUC | SSSS |
| WV- | mU*SGsm01n006Csm01n006Csm01n006mA*SG*SG*RC*ST*SG*RG*ST* | UGCCAGGCTGGTTATG | SnXnXnXSSRSSRSSRSS |
| 44365 | ST*RA*ST*SmG*SmA*SmC*SmU*SmC | ACUC | SSSS |
| WV- | mU*SGsm01n020Csm01n020Csm01n020mA*SG*SG*RC*ST*SG*RG*ST* | UGCCAGGCTGGTTATG | SnXnXnXSSRSSRSSRSS |
| 44366 | ST*RA*ST*SmG*SmA*SmC*SmU*SmC | ACUC | SSSS |
| WV- | mU*SGsm01*n002Csm01*n002Csm01*n002mA*SG*SG*RC*ST*SG*RG* | UGCCAGGCTGGTTATG | Sn*Xn*Xn*XSSRSSRSS |
| 44367 | ST*ST*RA*ST*SmG*SmA*SmC*SmU*SmC | ACUC | RSSSSSS |
| WV- | mU*SGsm01*n006Csm01*n006Csm01*n006mA*SG*SG*RC*ST*SG*RG* | UGCCAGGCTGGTTATG | Sn*Xn*Xn*XSSRSSRSS |
| 44368 | ST*ST*RA*ST*SmG*SmA*SmC*SmU*SmC | ACUC | RSSSSSS |
| WV- | mU*SGsm01*n020Csm01*n020Csm01*n020mA*SG*SG*RC*ST*SG*RG* | UGCCAGGCTGGTTATG | Sn*Xn*Xn*XSSRSSRSS |
| 44369 | ST*ST*RA*ST*SmG*SmA*SmC*SmU*SmC | ACUC | RSSSSSS |
| WV- | mU*SGeon029Rm5Ceon029Rm5Ceon029RmA*SG*SG*RC*ST*SG*RG*ST* | UGCCAGGCTGGTFATG | SnRnRnRSSRSSRSSRSS |
| 44370 | S1'*RA*ST*SmG*SmA*SmC*SmU*SmC | ACUC | SSSS |
| WV- | mU*SGcon001Rm5Ccom5CcomA*SG*SG*RC*STSG*RG*ST*ST*RA*ST* | UGCCAGGCTGGTATGA | SOROOSSRSSRSSRSSSS |
| 44468 | SmG*SmA*SmC*SmU*SmC | CUC | SS |
| WV- | mU*SGeon051Rm5Ceom5CeomA*SG*SG*RC*ST*SG*RG*ST*ST*RA*ST* | UGCCAGGCTGGTTATG | SnROOSSRSSRSSRSSSS |
| 44469 | SmG*SmA*SmC*SmU*SmC | ACUC | SS |
| WV- | mU*SGeon052Rm5Ceom5CeomA*SG*SG*RC*ST*SG*RG*ST*ST*RA*ST* | UGCCAGGCTGGTTATG | SnROOSSRSSRSSRSSSS |
| 44470 | SmG*SmA*SmC*SmU*SmC | ACUC | SS |
| WV- | mU*SGeon001RmSCeo*Sm5Ceon001RmA*SG*SG*RC*ST*SG*RG*ST*ST* | UGCCAGGCTGGTTATG | SnRSnRSSRSSRSSRSSS |
| 44962 | RA*ST*SmG*SmAn001RmC*SmU*SmC | ACUC | nRSS |
| WV- | mU*SGeon012Rm5Ceo*SmSCeon012RmA*SG*SG*RC*ST*SG*RG*ST*ST* | UGCCAGGCTGGTTATG | SnRSnRSSRSSRSSRSSS |
| 44964 | RA*ST*SmG*SmAn012RmC*SmU*SmC | ACUC | nRSS |
| WV- | mU*SGeom5Ceo*Sm5CeomA*SG*SG*RC*ST*SG*RG*ST*ST*RA*ST*SmG* | UGCCAGGCTGGTTATG | SOSOSSRSSRSSRSSSOS |
| 44966 | SmAmC*SmU*SmC | ACUC | S |
| WV- | mU*SGcon034Rm5Ccon034Rm5Ceon034RmA*SG*SG*RC*ST*SG*RG*ST* | UGCCAGGCTGGTTATG | SnRnRnRSSRSSRSSRSS |
| 45083 | ST*RA*ST*SmG*SmA*SmC*SmU*SmC | ACUC | SSSS |
| WV- | mU*SGcon035Rm5Ceon035Rm5Ceon@5RmA*SG*SG*RC*ST*SG*RG*ST* | UGCCAGGCTGGTTATG | SnRnRnRSSRSSRSSRSS |
| 45084 | ST*RA*ST*SmG*SmA*SmC*SmU*SmC | ACUC | SSSS |
| WV- | mU*SGcon041Rm5Ccon04LRm5Ceon041RmA*SG*SG*RC*ST*SG*RG*ST* | UGCCAGGCTGGTTATG | SnRnRnRSSRSSRSSRSS |
| 45085 | S1'*RA*ST*SmG*SmA*SmC*SmU*SmC | ACUC | SSSS |
| WV- | mU*SGeon043Rm5Ceon043Rm5Ceon043RmA*SG*SG*RC*ST*SG*RG*ST* | UGCCAGGCTGGTTATG | SnRnRnRSSRSSRSSRSS |
| 45086 | ST*RA*ST*SmG*SmA*SmC*SmU*SmC | ACUC | SSSS |
| WV- | mU*SGeon046Rm5Ceon046Rm5Ceon046RmA*SG*SG*RC*ST*SG*RG*ST* | UGCCAGGCTGGTTATG | SnRnRnRSSRSSRSSRSS |
| 45087 | ST*RA*ST*SmG*SmA*SmC*SmU*SmC | ACUC | SSSS |
| WV- | mU*SGeon047Rm5Ceon047Rm5Ceon047RmA*SG*SG*RC*ST*SG*RG*ST* | UGCCAGGCTGGTTATG | SnRnRnRSSRSSRSSRSS |
| 45088 | ST*RA*ST*SmG*SmA*SmC*SmU*SmC | ACUC | SSSS |
| WV- | mU*SGeon057Rm5Ceom5CeomA*SG*SG*RC*ST*SG*RG*ST*ST*RA*ST* | UGCCAGGCTGGTTATG | SnROOSSRSSRSSRSSSS |
| 45140 | SmG*SmA*SmC*SmU*SmC | ACUC | SS |
| WV- | mU*SGeom5Ceom5CeomA*SG*SG*RC*ST*SG*RG*ST*ST*RA*ST*SmG* | UGCCAGGCTGGTTATG | SOOOSSRSSRSSRSSSSS |
| 8587 | SmA*SmC*SmU*SmC | ACUC | S |
| WV- | mUn001Geom5Ceo®Sm5Ceon001mA*SG*SG*RC*ST*SG*RG*ST*ST*RA* | UGCCAGGCTGGTTATG | nXOSnXSSRSSRSSRSSS |
| 37974 | ST*SmG*SAsm01n013mC*SmU*SmC | ACUC | OSS |
| WV- | mU*SGeom5Ceom5CeoAsm01n0130*SG*RC*ST*SG*RG*ST*ST*RA*ST* | UGCCAGGCTGGTTATG | SOOOOSRSSRSSRSSSnX |
| 37977 | SmG*SmAn001mC*SmU*SmC | ACUC | SS |
| WV | fCn001RfU*S1C*SfU*SfG*SfG*S1G*S1C*S1A*S1A*S1A*S1G*SfA* | CUCUGGGCAAAGACAG | nRSSSSSSSSSSSSnRSn |
| 43086 | SfCn001R1A*SmGn001RmA*SmG*SmG*SmA*SmG*SmU*SmC*SfC*SC* | AGGAGUCCCCCUCG | RSSSSSSSSSnRSSnR |
| | SCn001RmC*SmU*SmCn001RmG | | |
| Wy- | mU*SGcon001Sm5Ceon001Sm5Ceon001SmA*SG*SG*RC*ST*SG*RG*ST* | UGCCAGGCTGGTTATG | SaSaSaSSSRSSRSSRSS |
| 15563 | ST*RA*ST*SmG*SmA*SmC*SmU*SmC | ACUC | SSSS |
| WV- | mU*SGsm01n001RCsm01n001RCsm01n001RmA*SG*SG*RC*ST*SG*RG* | UGCCAGGCTGGTTATG | SnRnRnRSSRSSRSSRSS |
| 43083 | ST*ST*RA*ST*SmG*SmA*SmC*SmU*SmC | ACUC | SSSS |
| WV- | mU*SGsm01n001SCsm01n001SCsm01n001SmA*SG*SG*RC*ST*SG*RG* | UGCCAGGCTGGTATGA | SOSOSOSSSRSSRSSRSS |
| 43084 | ST*ST*RA*ST*SmG*SmA*SmC*SmU*SmC | CUC | SSSS |
| wV- | mU*SGcom5CeoCsm01n013mA*SG*SG*RC*ST*SG*RG*ST*ST*RA*ST* | UGCCAGGCTGGTTATG | SOOOSSRSSRSSRSSSSS |
| 40557 | SmG*SmA*SmC*SmU*SmC | ACUC | S |
| WV- | mU*SGsm01n0BCsm0in013Csm01n013mA*SG*SG*RC*ST*SG*RG*ST* | UGCCAGGCTGGTTATG | SOOOSSRSSRSSRSSSSS |
| 40558 | ST*RA*ST*SmG*SmA*SmC*SmU*SmC | ACUC | S |
| WV- | mU*SGsm01n013m5CeoCsm0in013mA*SG*SG*RC*ST*SG*RG*ST*ST* | UGCCAGGCTGGTTATG | SOOOSSRSSRSSRSSSSS |
| 40559 | RA*ST*SmG*SmA*SmC*SmU*SmC | ACUC | S |
| WV- | mU*SGsm01n013m5CeoCsm01n013mA*SG*SG*RC*ST*SG*RG*ST*ST* | UGCCAGGCTGGTTATG | SOOOSSRSSRSSRSSSOS |
| 40560 | RA*ST*SmG*SAsm01n013mC*SmU*SmC | ACUC | S |
| WV- | mU*SGeom5Ceom5CeomA*SG*SGsm01n013C*ST*SG*RG*ST*ST*RA*ST* | UGCCAGGCTGGTTATG | SOOOSSOSSRSSRSSSSS |
| 40561 | SmG*SmA*SmC*SmU*SmC | ACUC | S |
| WV- | mU*SGcon001Rm5Ceon001Rm5Ceon001RmA*SG*SGsm01n013C*ST*SG* | UGCCAGGCTGGTTATG | SnRnRnRSSOSSRSSRSS |
| 40562 | RG*ST*ST*RA*ST*SmG*SmA*SmC*SmU*SmC | ACUC | SSSS |
| WV- | mU*SGeon001m5Ceon001m5Ceon001mA*SG*SG*RC*ST*SG*RG*ST*ST* | UGCCAGGCTGGTTATG | SnXnXnXSSRSSRSSRSS |
| 40563 | RA*ST*SmG*SAsm01n013mC*SmU*SmC | ACUC | SOSS |
| WV- | Mod038L001mU*SGeom5Ceom5CeomA*SG*SG*RC*ST*SG*RG*ST*ST*RA | UGCCAGGCTGGTTATG | OSOOOSSRSSRSSRSSSS |
| 39603 | *ST*SmG*SmA*SmC*SmU*SmC | ACUC | SS |
| WV- | Mod152L001mU*SGeom5Ceom5CeomA*SG*SG*RC*ST*SG*RG*ST*ST*RA | UGCCAGGCTGGTTATG | OSOOOSSRSSRSSRSSSS |
| 39604 | *ST*SmG*SmA*SmC*SmU*SmC | ACUC | SS |
| WV- | Mod154L001mU*SGeom5Ceom5CeomA*SG*SG*RC*ST*SG*RG*ST*ST*RA | UGCCAGGCTGGTTATG | OSOOOSSRSSRSSRSSSS |
| 39605 | *ST*SmG*SmA*SmC*SmU*SmC | ACUC | SS |
| WV- | Mod155U001mU*SGeom5Ceom5CeomA*SG*SG*RC*ST*SG*RG*ST*ST*RA | UGCCAGGCTGGTTATG | OSOOOSSRSSRSSRSSSS |
| 39601 | *ST*SmG*SmA*SmC*SmU*SmC | ACUC | SS |
| WV- | mC*Sm5Ceon001Teon001Teon001mC*SC*SC*RT*SG*SA*RA*SG*SG*RT | CCTTCCCTGAAGGTTC | SnXnXnXSSRSSRSSRSS |
| 16373 | *ST*SmC*SmC*SmU*SmC*SmC | CUCC | SSSS |
| WV- | mU*Geom5Ceom5CeomA*G*G*C*T*G*G*T*T*A*T*mG*mA*mCmU*mC | UGCCAGGCTGGTTATG | XOOOXXXXXXXXXXXXXX |
| 8556 | | ACUC | X |
| WV- | mU*SGeon001Rm5Ceon001SmSCeon001SmA*SG*SG*RC*ST*SG*RG*ST* | UGCCAGGCTGGTTATG | SnRnSnSSSRSSRSSRSS |
| 32687 | ST*RA*ST*SmG*SmA*SmC*SmU*SmC | ACUC | SSSS |
| WV- | mU*SGeon001Rm5Ceon001Rm5Ceon001SmA*SG*SG*RC*ST*SG*RG*ST* | UGCCAGGCTGGTTATG | SnRnRnSSSRSSRSSRSS |
| 32688 | ST*RA*ST*SmG*SmA*SmC*SmU*SmC | ACUC | SSSS |
| WV- | mU*SGeon001Sm5Ceon001Rm5Ceon001RmA*SG*SG*RC*ST*SG*RG*ST* | UGCCAGGCTGGTTATG | SnSnRnRSSRSSRSSRSS |
| 32689 | ST*RA*ST*SmG*SmA*SmC*SmU*SmC | ACUC | SSSS |
| WV- | mU*SGcon001Sm5Ceon001Sm5Ceon001RmA*SG*SG*RC*ST*SG*RG*ST* | UGCCAGGCTGGTTATG | SnSnSnRSSRSSRSSRSS |
| 32690 | S1'*RA*ST*SmG*SmA*SmC*SmU*SmC | ACUC | SSSS |
| WV- | mU*SGcon001Rm5Ceon001Sm5Ccon001RmA*SG*SG*RC*ST*SG*RG*ST* | UGCCAGGCTGGTATGA | SORnSnRSSRSSRSSRSS |
| 32691 | ST*RA*ST*SmG*SmA*SmC*SmU*SmC | CUC | SSSS |
| WV- | mU*SGcon001Sm5Ceon001Rm5Ceon001SmA*SG*SG*RC*ST*SG*RG*ST* | UGCCAGGCTGGTTATG | SnSnRnSSSRSSRSSRSS |
| 32692 | ST*RA*ST*SmG*SmA*SmC*SmU*SmC | ACUC | SSSS |
| WV- | mU*SGeon001m5Ceom5Ceon001mA*SG*SG*RC*ST*SG*RG*ST*ST*RA* | UGCCAGGCTGGTTATG | SnXOnXSSRSSRSSRSSS |
| 30915 | ST*SmG*SmA*SmC*SmUn001mC | ACUC | SSnX |
| WV- | mU*SGeon001m5Ceom5Ceon001mA*SG*SG*RC*ST*SG*RG*ST*ST*RA* | UGCCAGGCTGGTTATG | SnXOnXSSRSSRSSRSSS |
| 30916 | ST*SmG*SmAn001mC*SmU*SmC | ACUC | nXSS |
| WV- | mU*SGeon001Rm5CeomSCeon001RmA*SG*SG*RC*ST*SG*RG*ST*ST*RA | UGCCAGGCTGGTTATG | SnROnRSSRSSRSSRSSS |
| 38634 | *ST*SmG*SmA*SmC*SmUn001RmC | ACUC | SSnR |
| WV | mU*SGeon001Sm5CeomSCeon001SmA*SG*SG*RC*ST*SG*RG*ST*ST*RA | UGCCAGGCTGGTTATG | SnSOnSSSRSSRSSRSSS |
| 38635 | *ST*SmG*SmA*SmC*SmUn001SmC | ACUC | SSnS |
| WV- | mU*SGcon001m5Ceom5Ceon001mA*SG*SG*RC*ST*SG*RG*ST*ST*RA* | UGCCAGGCTGGTTATG | SnXOnXSSRSSRSSRSSn |
| 38636 | ST*SmGn001mA*SmC*SmUn001mC | ACUC | XSSnX |
| WV- | mU*SGcon001RmSCeom5Ccon001RmA*SG*SG*RC*ST*SG*RG*ST*ST*RA | UGCCAGGCTGGTTATG | SnROnRSSRSSRSSRSSn |
| 38637 | *ST*SmGn001RmA*SmC*SmUn001RmC | ACUC | RSSnR |
| WV- | mU*SGcon001Sm5Ccom5Ceon001SmA*SG*SG*RC*ST*SG*RG*ST*ST*RA | UGCCAGGCTGGTTATG | SnSOnSSSRSSRSSRSSn |
| 38638 | *ST*SmGn001SmA*SmC*SmUn001SmC | ACUC | SSSnS |
| WV- | rGrArGrUrCrArUrArArCrCrArGrCrCrUrGrGrCrA | GAGUCAUAACCAGCCU | OOOOOOOOOOOOOOOOOO |
| 6573 | | GGCA | O |

TABLE A4
Example oligonucleotides and/or compositions.

| | | | Stereochemistry/ |
|---|---|---|---|
| ID | Description | Base Sequence | Linkage |
| WV- | mC*m5CeoTeoTeomC*C*C*T*G*A* | CCTTCCCTGAAGG | XOOOXXXXXXXXXXX |
| 9491 | A*G*G*T*T*mC*mC*mU*mC*mC | TTCCUCC | XXXX |

### Notes:

Description, Base Sequence and Stereochemistry/Linkage, due to their length, may be divided into multiple lines in Tables A1-A4 (e.g., Table A1, Table A2, Table A3 and Table A4). Unless otherwise specified, all oligonucleotides in Table A1-A4 are single-stranded. As appreciated by those skilled in the art, nucleoside units are unmodified and contain unmodified nucleobases and 2′-deoxy sugars unless otherwise indicated (e.g., with r, m, m5, eo, etc.; if not indicated, a sugar is a natural DNA sugar); linkages, unless otherwise indicated, are natural phosphate linkages; and acidic/basic groups may independently exist in their salt forms. Moieties and modifications:
m: 2′-OMe;
m5: methyl at 5-position of C (nucleobase is 5-methylcytosine);
1: LNA sugar;
f: 2′-F;
eo: 2′-MOE (2′-OCH₂CH₂OCH₃);
m5Ceo: 5-methyl 2′-O-methoxyethyl C;
O, PO: phosphodiester (phosphate). It can be a linkage, e.g., a linkage between a linker and an oligonucleotide chain, an internucleotidic linkage (a natural phosphate linkage), etc. Phosphodiesters are typically indicated with “0” in the Stereochemistry/Linkage column and are typically not marked in the Description column (if it is an end group, e.g., a 5′-end group, it is indicated in the Description and typically not in Stereochemistry/Linkage); if no linkage is indicated in the Description column, it is typically a phosphodiester unless otherwise indicated. Note that a phosphate linkage between a linker (e.g., L001) and an oligonucleotide chain may not be marked in the Description column, but may be indicated with “O” in the Stereochemistry/Linkage column;
*, PS: Phosphorothioate. It can be a linkage, e.g., a linkage between linker (e.g., L001) and an oligonucleotide chain, an internucleotidic linkage (a phosphorothioate internucleotidic linkage), etc.;
R, Rp: Phosphorothioate in the Rp configuration. Note that * R in Description indicates a single phosphorothioate linkage in the Rp configuration;
S, Sp: Phosphorothioate in the Sp configuration. Note that * S in Description indicates a single phosphorothioate linkage in the Sp configuration;
X: stereorandom phosphorothioate;
n001:

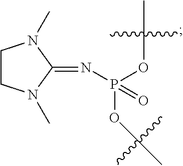 [SMILES: *OP(=O)(N=C1N(C)CCN1C)O*]

nX(when utilized for n001): stereorandom n001;
nR (when utilized for n001) or n001R: n001 in Rp configuration;
nS (when utilized for n001) or n001S: n001 in Sp configuration;
sm01n001

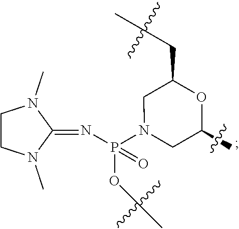

n002:

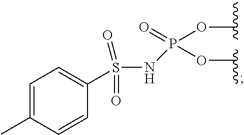 [SMILES: *OP(=O)(NS(=O)(=O)c1ccc(C)cc1)O*]

nX(when utilized for n002): stereorandom n002;
nR(when utilized for n002) or n002R: n002 in Rp configuration;
nS (when utilized for n002) or n002S: n002 in Sp configuration;
n003:

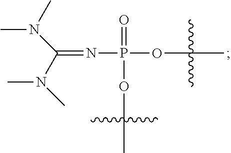 [SMILES: *OP(=O)(N=C(N(C)C)N(C)C)O*]

nX(when utilized for n003): stereorandom n003;
nR(when utilized for n003) or n003R: n003 in Rp configuration;
nS (when utilized for n003) or n003S: n003 in Sp configuration;
n004:

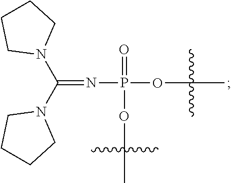 [SMILES: *OP(=O)(N=C(N1CCCC1)N1CCCC1)O*]

nX(when utilized for n004): stereorandom n004;
nR(when utilized for n004) or n004R: n004 in Rp configuration;
nS (when utilized for n004) or n004S: n004 in Sp configuration;
n006:

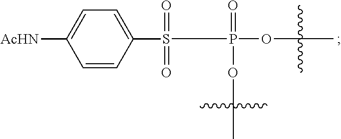 [SMILES: *OP(=O)(CS(=O)(=O)c1ccc(NC(C)=O)cc1)O*]

nX(when utilized for n006): stereorandom n006;
nR(when utilized for n006) or n006R: n006 in Rp configuration;
nS (when utilized for n006) or n006S: n006 in Sp configuration;
n008:

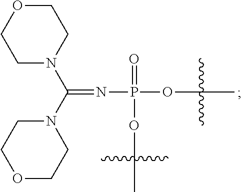 [SMILES: *OP(=O)(N=C(N1CCOCC1)N1CCOCC1)O*]

nX(when utilized for n008): stereorandom n008;
nR(when utilized for n008) or n008R: n008 in Rp configuration;
nS (when utilized for n008) or n008S: n008 in Sp configuration;
n009:

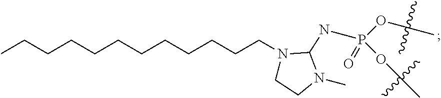 [SMILES: *OP(=O)(NC1N(C)CCN1CCCCCCCCCCCC)O*]

nX(when utilized for n009): stereorandom n009;
nR(when utilized for n009) or n009R: n009 in Rp configuration;
nS (when utilized for n009) or n009S: n009 in Sp configuration;
n012:

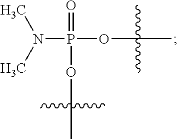 [SMILES: *OP(=O)(O*)N(C)C]

nX(when utilized for n012): stereorandom n012;
nR(when utilized for n012) or n012R: n012 in Rp configuration;
nS (when utilized for n012) or n012S: n012 in Sp configuration;
n020:

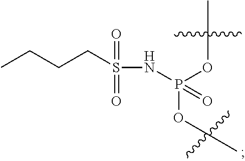 [SMILES: *OP(=O)(NS(=O)(=O)CCCC)O*]

nX(when utilized for n020): stereorandom n020;
nR(when utilized for n020) or nO2OR: n020 in Rp configuration;
nS (when utilized for n020) or n020S: n020 in Sp configuration;
n021:

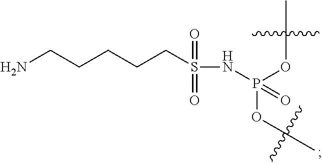 [SMILES: *OP(=O)(NS(=O)(=O)CCCCCN)O*]

nX(when utilized for n021): stereorandom n021;
nR(when utilized for n021) or n021R: n021 in Rp configuration;
nS (when utilized for n021) or n021S: n021 in Sp configuration;
n025:

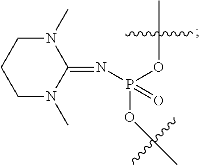 [SMILES: *OP(=O)(N=C1N(C)CCCN1C)O*]

nX(when utilized for n025): stereorandom n025;
nR(when utilized for n025) or n025R: n025 in Rp configuration;
nS (when utilized for n025) or n025S: n025 in Sp configuration;
n026:

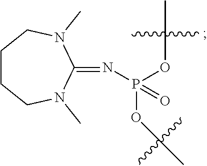 [SMILES: *OP(=O)(N=C1N(C)CCCCN1C)O*]

nX(when utilized for n026): stereorandom n026;
nR(when utilized for n026) or n026R: n026 in Rp configuration;
nS (when utilized for n026) or n026S: n026 in Sp configuration;
n029:

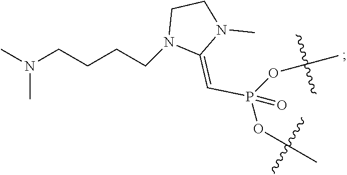 [SMILES: *OP(=O)(/C=C1\N(C)CCN1CCCCN(C)C)O*]

nX(when utilized for n029): stereorandom n029;
nR(when utilized for n029) or n029R: n029 in Rp configuration;
nS (when utilized for n029) or n029S: n029 in Sp configuration;
n030:

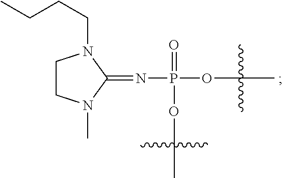 [SMILES: *OP(=O)(N=C1N(C)CCN1CCCC)O*]

nX(when utilized for n030): stereorandom n030;
nR(when utilized for n030) or n030R: n030 in Rp configuration;
nS (when utilized for n030) or n030S: n030 in Sp configuration;
n031:

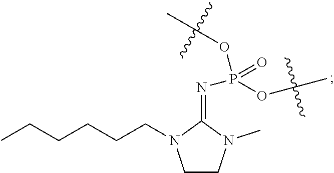 [SMILES: *OP(=O)(/N=C1\N(C)CCN1CCCCCC)O*]

nX(when utilized for n031): stereorandom n031;
nR(when utilized for n031) or n031R: n031 in Rp configuration;
nS (when utilized for n031) or n031S: n031 in Sp configuration;
n033:

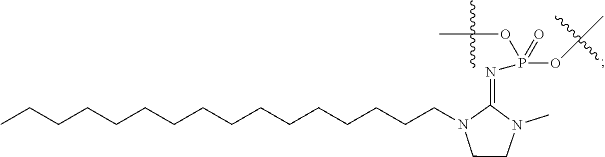

nX(when utilized for n033): stereorandom n033;
nR(when utilized for n033) or n033R: n033 in Rp configuration;
nS (when utilized for n033) or n033S: n033 in Sp configuration;
n034:

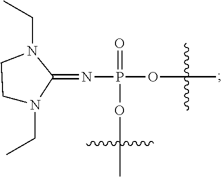

nX(when utilized for n034): stereorandom n034;
nR(when utilized for n034) or n034R: n034 in Rp configuration;
nS (when utilized for n034) or n034S: n034 in Sp configuration;
n035:

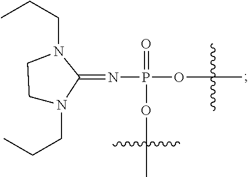

nX(when utilized for n035): stereorandom n035;
nR(when utilized for n035) or n035R: n035 in Rp configuration;
nS (when utilized for n035) or n035S: n035 in Sp configuration;
n036:

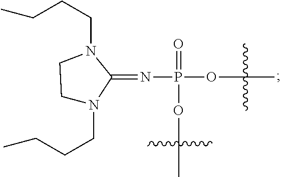

nX(when utilized for n036): stereorandom n036;
nR(when utilized for n036) or n036R: n036in Rp configuration;
nS (when utilized for n036) or n036S: n036 in Sp configuration;
n037:

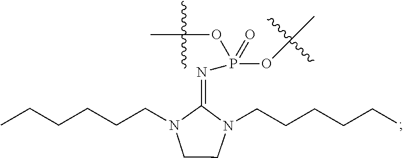

nX(when utilized for n037): stereorandom n037;
nR(when utilized for n037) or n037R: n037in Rp configuration;
nS (when utilized for n037) or n037S: n037 in Sp configuration;
n041:

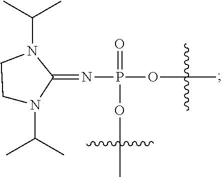

nX(when utilized for n041): stereorandom n041;
nR(when utilized for n041) or n041R: n04lin Rp configuration;
nS (when utilized for n041) or n041S: n041 in Sp configuration;
n043:

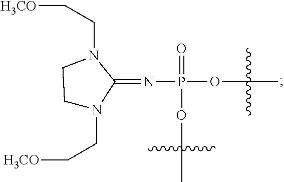

nX(when utilized for n043): stereorandom n043;
nR(when utilized for n043) or n043R: n043in Rp configuration;
nS (when utilized for n043) or n043S: n043 in Sp configuration;
n046:

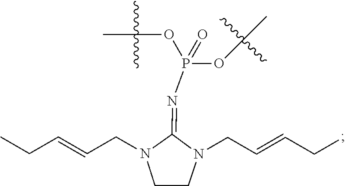

nX(when utilized for n046): stereorandom n046;
nR(when utilized for n046) or n046R: n046in Rp configuration;
nS (when utilized for n046) or n046S: n046 in Sp configuration;
n047:

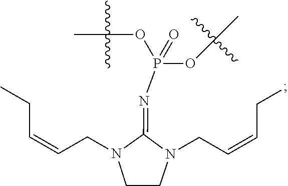

nX(when utilized for n047): stereorandom n047;
nR(when utilized for n047) or n047R: n047in Rp configuration;
nS (when utilized for n047) or n047S: n047 in Sp configuration;
n051:

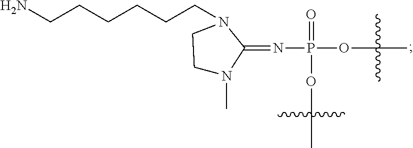

nX(when utilized for n051): stereorandom n051;
nR(when utilized for n051) or n051R: n051 in Rp configuration;
nS (when utilized for n051) or n051S: n051 in Sp configuration;
n052:

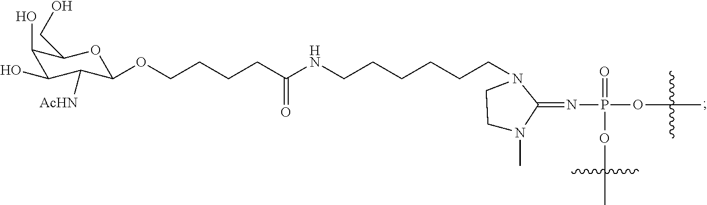

nX(when utilized for n052): stereorandom n052;
nR(when utilized for n052) or n052R: n052 in Rp configuration;
nS (when utilized for n052) or n052S: n052 in Sp configuration;
n054:

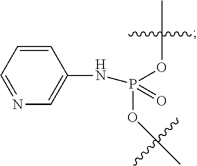

nX(when utilized for n054): stereorandom n054;
nR(when utilized for n054) or n054R: n054 in Rp configuration;
nS (when utilized for n054) or n054S: n054 in Sp configuration;
n055:

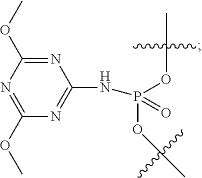

nX(when utilized for n055): stereorandom n055;
nR(when utilized for n055) or n055R: n055 in Rp configuration;
nS (when utilized for n055) or n055S: n055 in Sp configuration;
n057:

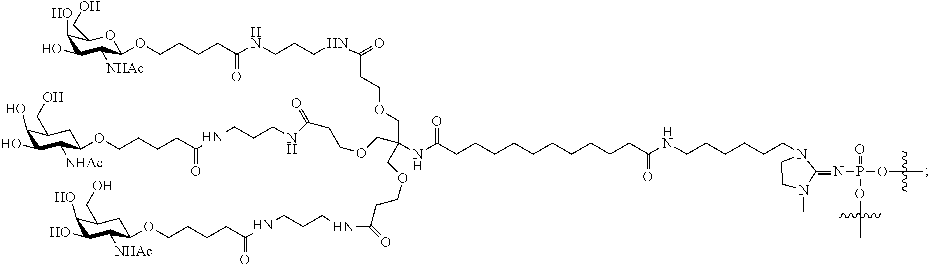

nX(when utilized for n057): stereorandom n057;
nR(when utilized for n057) or n057R: n057 in Rp configuration;
nS (when utilized for n057) or n057S: n057 in Sp configuration;

n013:

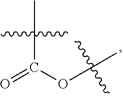

wherein —C(O)— is bonded to nitrogen. n013 may be indicated as 0 (e.g., for WV-40562, SnRnRnRSSOSSRSSRSSSSSS);
*n001:

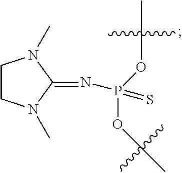 [SMILES: *OP(=S)(N=C1N(C)CCN1C)O*]

n*X (when utilized for *n001): stereorandom *n001;
*n002:

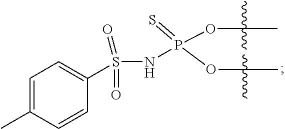 [SMILES: *OP(=S)(NS(=O)(=O)c1ccc(C)cc1)O*]

n*X (when utilized for *n002): stereorandom *n002;
*n006:

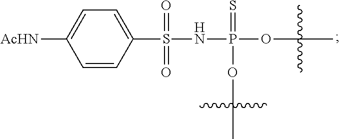 [SMILES: *OP(=S)(NS(=O)(=O)c1ccc(NC(C)=O)cc1)O*]

n*X (when utilized for *n006): stereorandom *n006;
*n020:

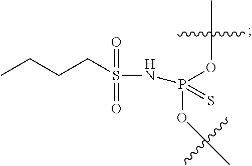 [SMILES: *OP(=S)(NS(=O)(=O)CCCC)O*]

n*X (when utilized for *n020): stereorandom *n020;
sm01:

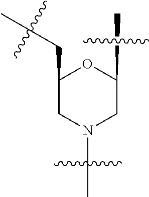

(for example, Gsm01 is

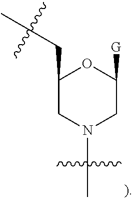

As appreciated by those skilled in the art, when sm01 is at the 5′-end, its —CH₂—may be bonded to a 5′-end group as for various other sugars (e.g., —OH as typically in many oligonucleotides unless indicated otherwise);

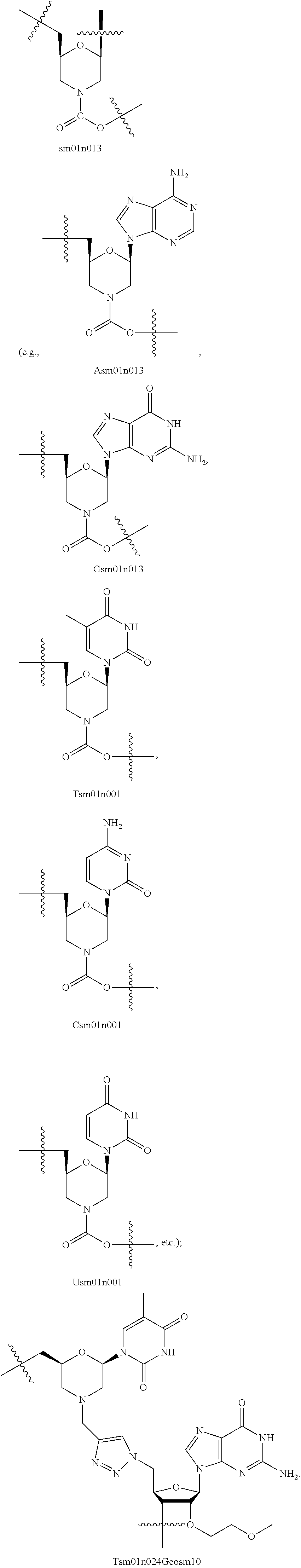

In some embodiments, the linkage in between is indicated as O (e.g., for WV-40835, the first O of OOOOSSRSSRSSRSSSSSS);
mUsm05Geosm06:

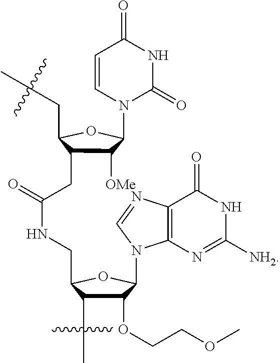

In some embodiments, the linkage in between is indicated as O (e.g., for WV-40807, the first O of OOOOSSRSSRSSRSSSSSS);
mUsm09Geosm10:

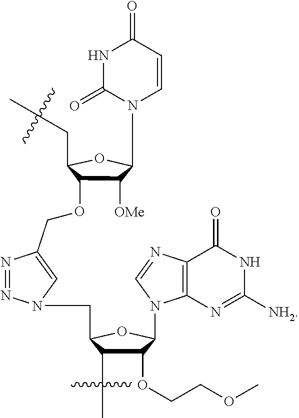

In some embodiments, the linkage in between is indicated as O (e.g., for WV-40808, the first O of OOOOSSRSSRSSRSSSSSS);
sm04:

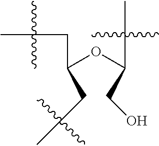

(for example, Usm04 is

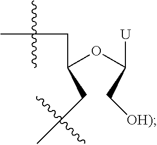

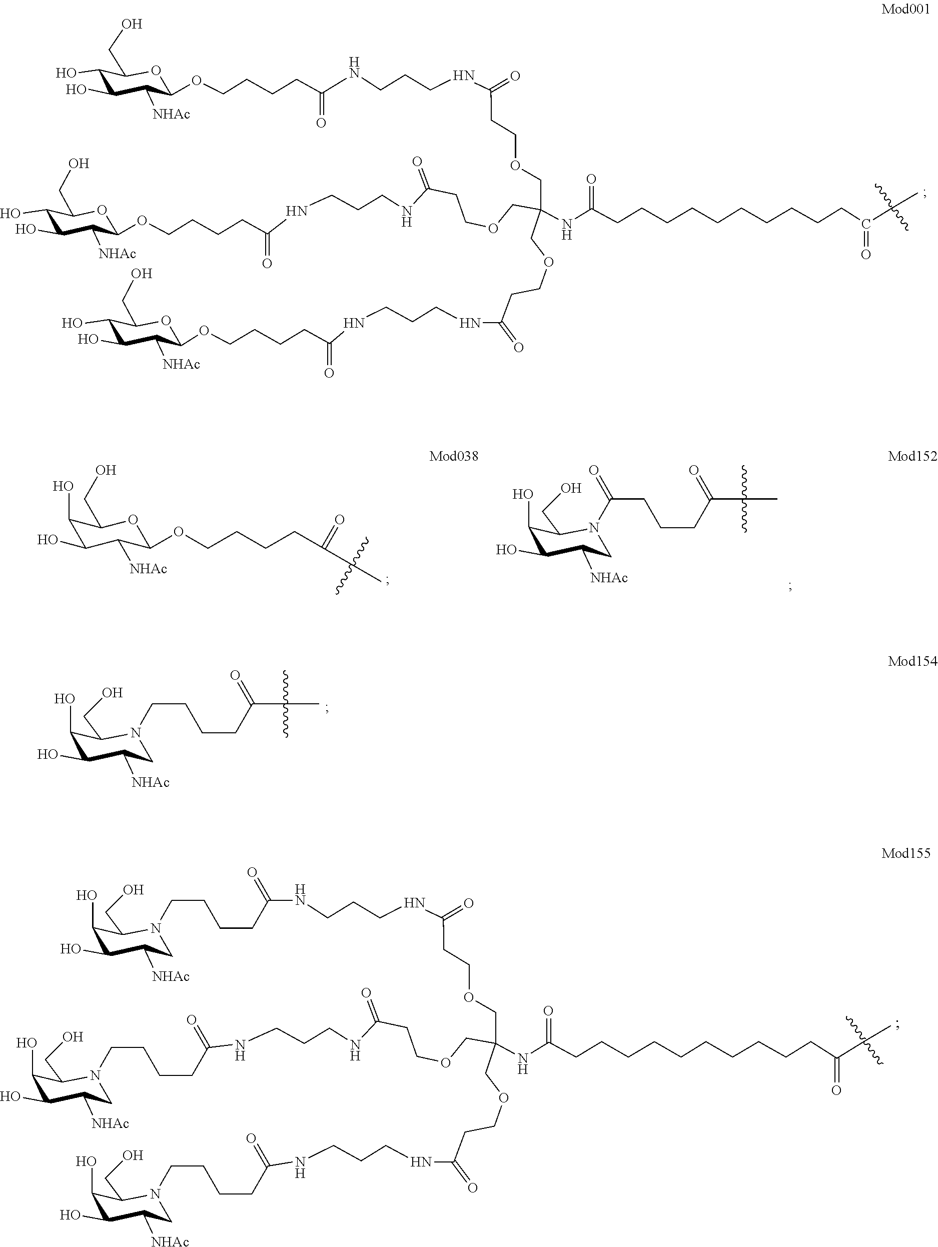

L001: —NH—(CH₂)₆—linker (C6 linker, C6 amine linker or C6 amino linker). —NH— is connected to Mod (e.g., Mod001; if no Mod, connected to —H), and —CH₂— is connected to the 5′-end of an oligonucleotide chain through phosphate unless indicated otherwise. For example, in WV-27457, L001 is connected to the 5′-carbon at the 5′-end of the oligonucleotide chain through a phosphate linkage (O or PO); L023: HO—(CH₂)₆—, wherein CH₂ is connected to the rest of a molecule through a phosphate unless indicated otherwise. For example, in WV-28763 (wherein the O in OnXnXnXSSSSSSOSSSSOOSSSSSS indicates a phosphate linkage connecting L023 to the rest of the molecule);
L009: —CH₂CH₂CH₂—. L009 connects to other moieties, e.g., L023, L009, oligonucleotide chains, etc., through various linkages (e.g., n001; if not indicated, typically phosphates). When no other moieties are present, L009 is bonded to —OH. For example, in WV-28763, L009 is utilized with n009 to form
L009n009, which has the structure of

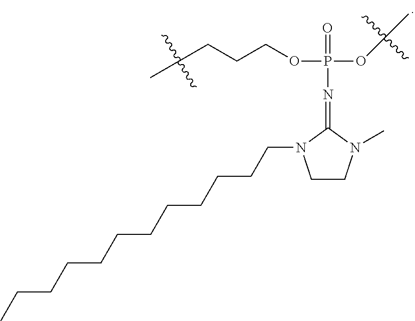 [SMILES: *CCCOP(=O)(N=C1N(C)CCN1CCCCCCCCCCCC)O*]

In some embodiments, multiple L009 may be utilized. For example, WV-28763 comprises L023L009n009L009n009L009n009, which has the following structure (which is bonded to the 5′-carbon at the 5′-end of the oligonucleotide chain):

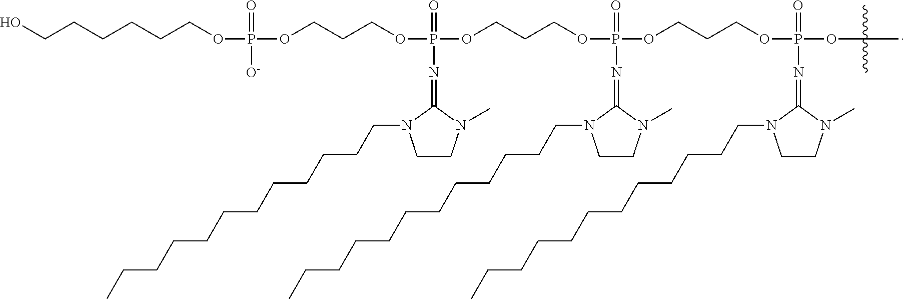 [SMILES: *OP(=O)(N=C1N(C)CCN1CCCCCCCCCCCC)OCCCOP(=O)(N=C1N(C)CCN1CCCCCCCCCCCC)OCCCOP(=O)(N=C1N(C)CCN1CCCCCCCCCCCC)OCCCOP(=O)([O-])OCCCCCCO]

In some embodiments, for example, in WV-23578, L009 is utilized with n001 to form L009n001, which has the structure of

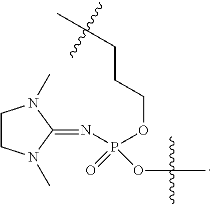 [SMILES: *CCCOP(=O)(N=C1N(C)CCN1C)O*]

In some embodiments, multiple L009n001 may be utilized. For example, WV-23578 comprises L009n001L009n001L009n001L009, which has the following structure (which is bonded to the 5′-carbon at the 5′-end of the oligonucleotide chain):

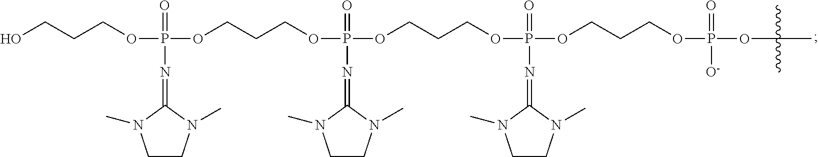 [SMILES: *OP(=O)([O-])OCCCOP(=O)(N=C1N(C)CCN1C)OCCCOP(=O)(N=C1N(C)CCN1C)OCCCOP(=O)(N=C1N(C)CCN1C)OCCCO]

L010:

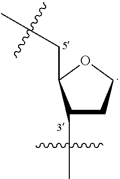

In some embodiments, when L010 is present in the middle of an oligonucleotide, it is bonded to internucleotidic linkages as other sugars (e.g., DNA sugars), e.g., its 5′-carbon is connected to another unit (e.g., 3′ of a sugar) and its 3′-carbon is connected to another unit (e.g., a 5′-carbon of a carbon) independently, e.g., via a linkage (e.g., a phosphate linkage (O or PO) or a phosphorothioate linkage (can be either not chirally controlled or chirally controlled (Sp or Rp))). L010 connects to other moieties, e.g., L023, L010, oligonucleotide chains, etc., through various linkages (e.g., n001; if not indicated, typically phosphates). When no other moieties are present, L010 is bonded to —OH. For example in WV-28764, L010 is utilized with n009 to form L010n009, which has the structure of

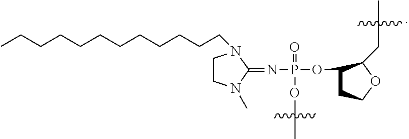

In some embodiments, multiple L010n009 may be utilized. For example, WV-28764 comprises L023L010n009L010n009L010n009, which has the following structure (which is bonded to the 5′-carbon at the 5′-end of the oligonucleotide chain):

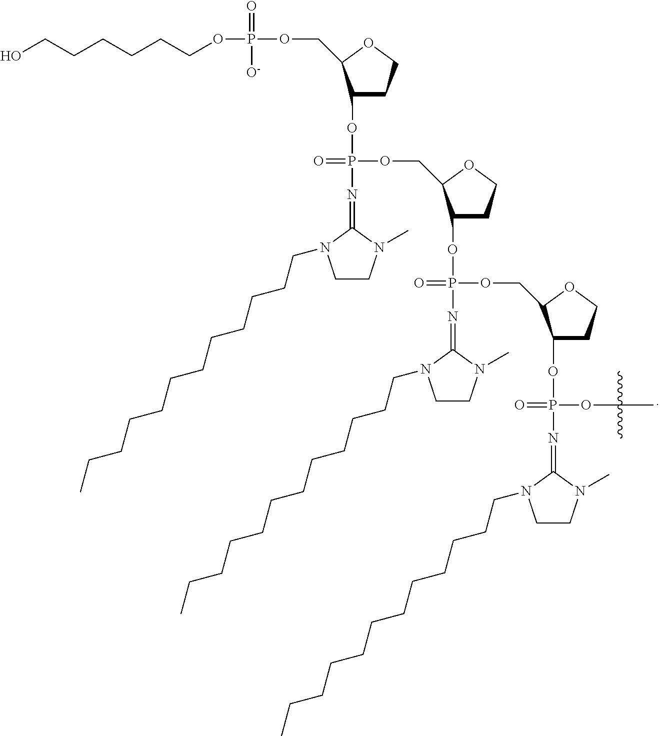

In some embodiments, for example, in WV-23938, L010 is utilized with n001 to form L010n001, which has the structure of

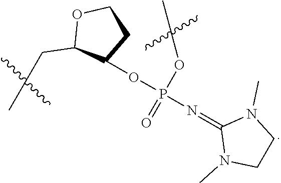

In some embodiments, multiple L010n001 may be utilized. For example, WV-23938 comprises L010n001L010n001L010n001L009 which has the following structure (which is bonded to the 5′-carbon at the 5′-end of the oligonucleotide chain):

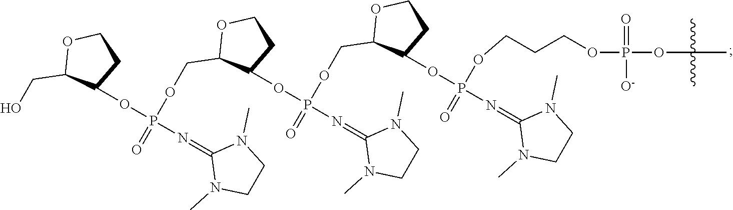

L012: —CH₂CH₂OCH₂CH₂OCH₂CH₂—. When L012 is present in the middle of an oligonucleotide, each of its two ends is independently bonded to an internucleotidic linkage (e.g., a phosphate linkage (O or PO) or a phosphorothioate linkage (can be either not chirally controlled or chirally controlled (Sp or Rp)));
L022:

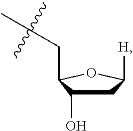

wherein L022 is connected to the rest of a molecule through a phosphate unless indicated otherwise;
L025:

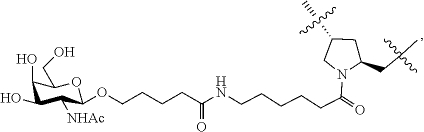

wherein the —CH₂—connection site is utilized as a C5 connection site of a sugar (e.g., a DNA sugar) and is connected to another unit (e.g., 3′ of a sugar), and the connection site on the ring is utilized as a C3 connection site and is connected to another unit (e.g., a 5′-carbon of a carbon), each of which is independently, e.g., via a linkage (e.g., a phosphate linkage (O or PO) or a phosphorothioate linkage (can be either not chirally controlled or chirally controlled (Sp or Rp))). When L025 is at a5′-end without any modifications, its —CH₂—connection site is bonded to —OH. For example, L025L025L025—in various oligonucleotides has the structure of

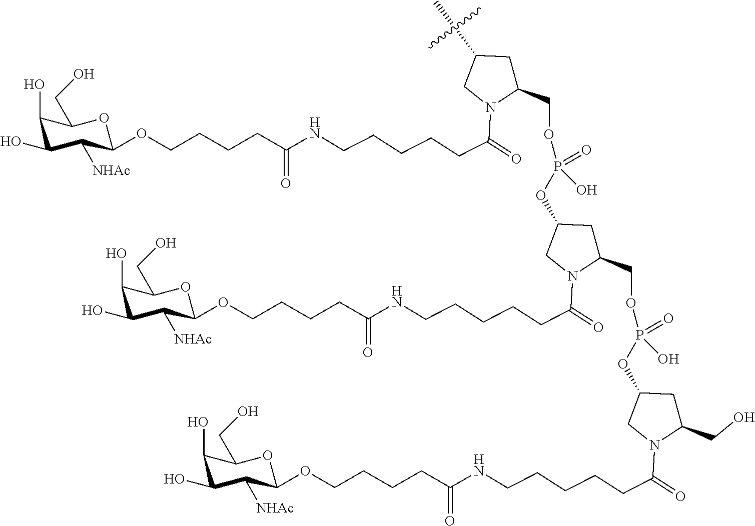

(may exist as various salt forms) and is connected to 5′-carbon of an oligonucleotide chain via a linkage as indicated (e.g., a phosphate linkage (O or PO) or a phosphorothioate linkage (can be either not chirally controlled or chirally controlled (Sp or Rp)));
L016:

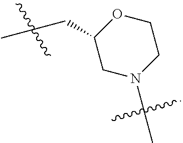 [SMILES: *C[C@H]1CN(*)CCO1]

In some embodiments, for example, in WV-28767, L016 is utilized with n001 to form L016n001, which has the structure of

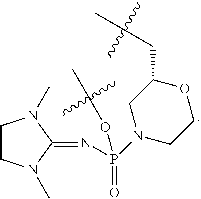 [SMILES: *C[C@H]1CN(P(=O)(N=C2N(C)CCN2C)O*)CCO1]

In some embodiments, multiple L016n001 may be utilized. For example, WV-28767 comprises L023L016n001L016n001L016n001, which has the following structure (which is bonded to the 5′-carbon at the 5′-end of the oligonucleotide chain):

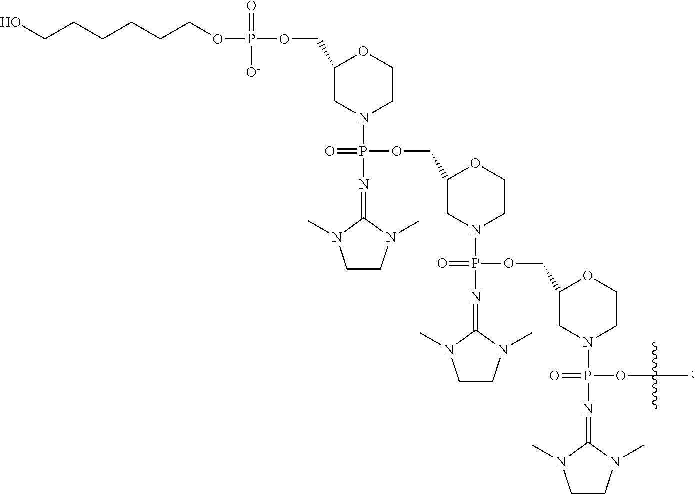

L017:

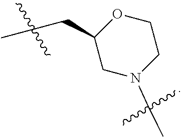 [SMILES: *C[C@@H]1CN(*)CCO1]

In some embodiments, for example, in WV-28768, L017 is utilized with n001 to form L017n001, which has the structure of

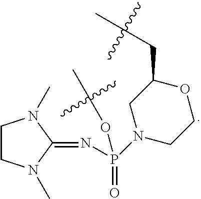 [SMILES: *C[C@@H]1CN(P(=O)(N=C2N(C)CCN2C)O*)CCO1]

In some embodiments, multiple L017n001 may be utilized. For example, WV-28768 comprises L023L017n001L017n001L017n001, which has the following structure (which is bonded to the 5′-carbon at the 5′-end of the oligonucleotide chain):

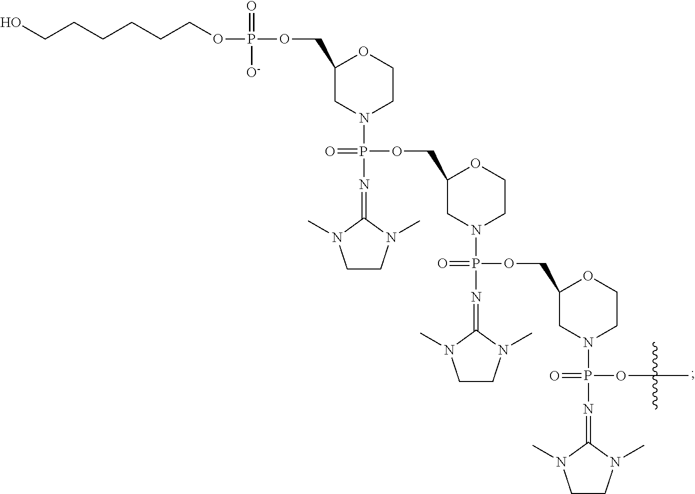 [SMILES: *OP(=O)(N=C1N(C)CCN1C)N1CCO[C@H](COP(=O)(N=C2N(C)CCN2C)N2CCO[C@H](COP(=O)(N=C3N(C)CCN3C)N3CCO[C@H](COP(=O)([O-])OCCCCCCO)C3)C2)C1]

L018:

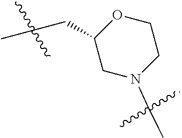 [SMILES: *C[C@H]1CN(*)CCO1]

In some embodiment, for example in WV-28765, L018 is utilized with n009 to form L018n009, which has the structure of

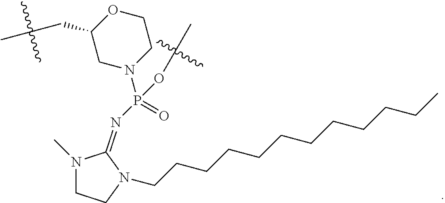 [SMILES: *C[C@H]1CN(P(=O)(/N=C2/N(C)CCN2CCCCCCCCCCCC)O*)CCO1]

In some embodiments, multiple L018n009 may be utilized. For example, WV-28765 comprises L023L018n009L018n009L018n009, which has the following structure (which is bonded to the 5′-carbon at the 5′-end of the oligonucleotide chain):

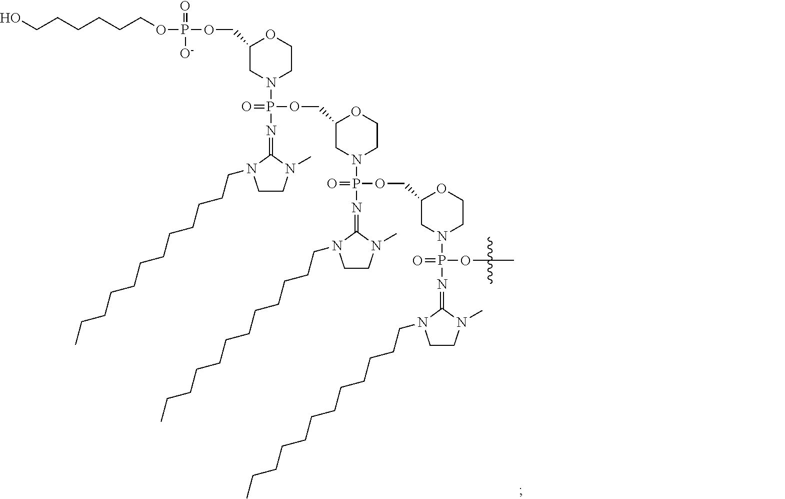 [SMILES: *OP(=O)(N=C1N(C)CCN1CCCCCCCCCCCC)N1CCO[C@@H](COP(=O)(N=C2N(C)CCN2CCCCCCCCCCCC)N2CCO[C@@H](COP(=O)(N=C3N(C)CCN3CCCCCCCCCCCC)N3CCO[C@@H](COP(=O)([O-])OCCCCCCO)C3)C2)C1]

L019:

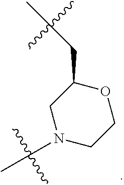 [SMILES: *C[C@@H]1CN(*)CCO1]

In some embodiments, for example, in WV-28766, L019 is utilized with n009 to form L019n009, which has the structure of

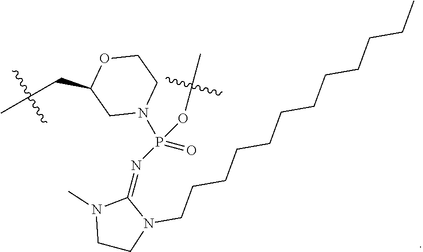 [SMILES: *C[C@@H]1CN(P(=O)(/N=C2/N(C)CCN2CCCCCCCCCCCC)O*)CCO1]

In some embodiments, multiple L019n009 may be utilized. For example, WV-28766 comprises L023L019n009L019n009L019n009, which has the following structure (which is bonded to the 5′-carbon at the 5′-end of the oligonucleotide chain):

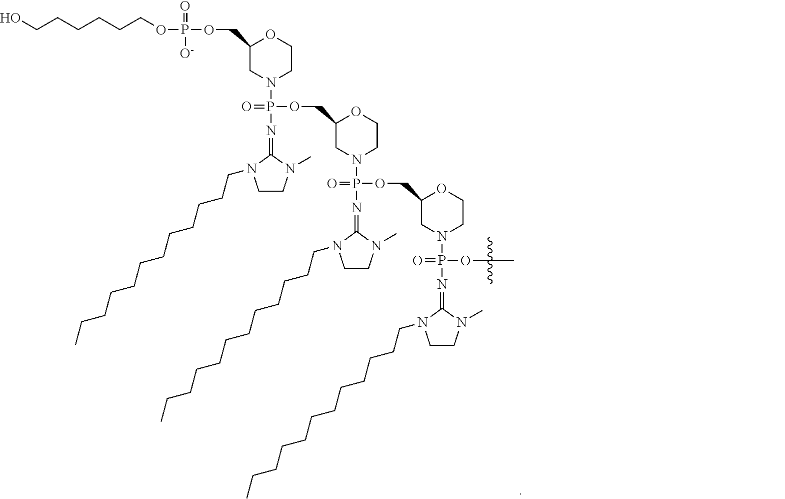 [SMILES: *OP(=O)(N=C1N(C)CCN1CCCCCCCCCCCC)N1CCO[C@H](COP(=O)(N=C2N(C)CCN2CCCCCCCCCCCC)N2CCO[C@H](COP(=O)(N=C3N(C)CCN3CCCCCCCCCCCC)N3CCO[C@H](COP(=O)([O-])OCCCCCCO)C3)C2)C1]

Structures of certain oligonucleotides are depicted below. Those skilled in the art will appreciate that they may be in various forms, e.g., various salt forms, particularly pharmaceutically acceptable salt forms. In some embodiments, the present disclosure provides the following compounds/oligonucleotides:

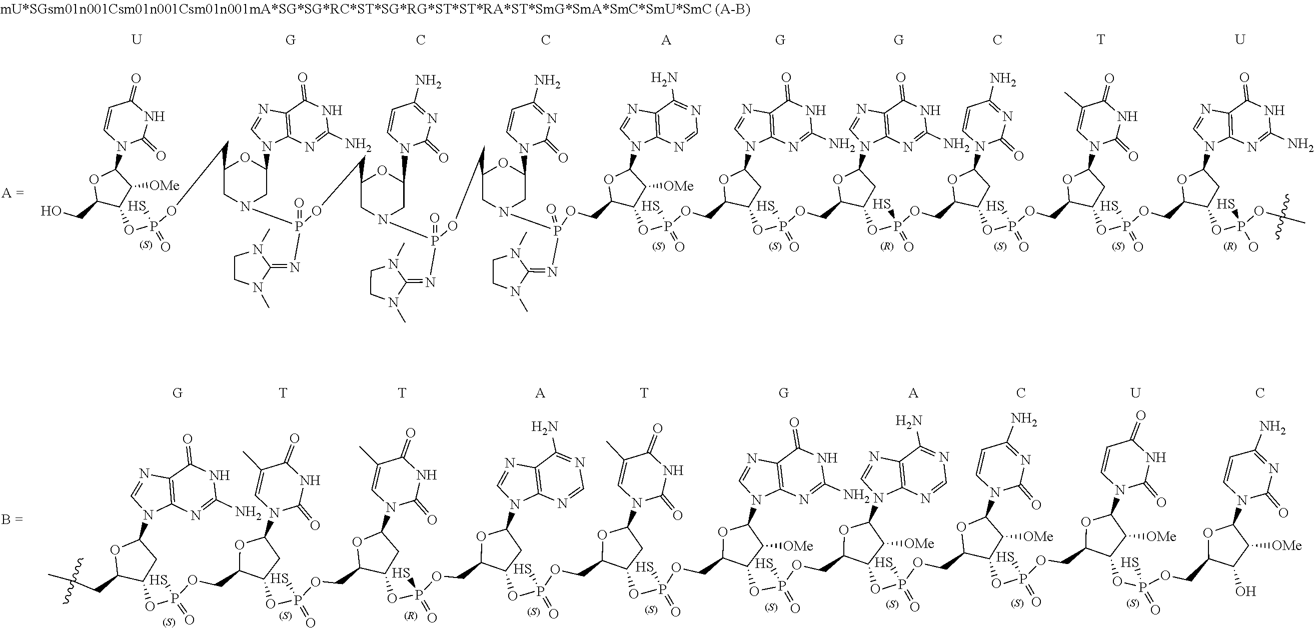

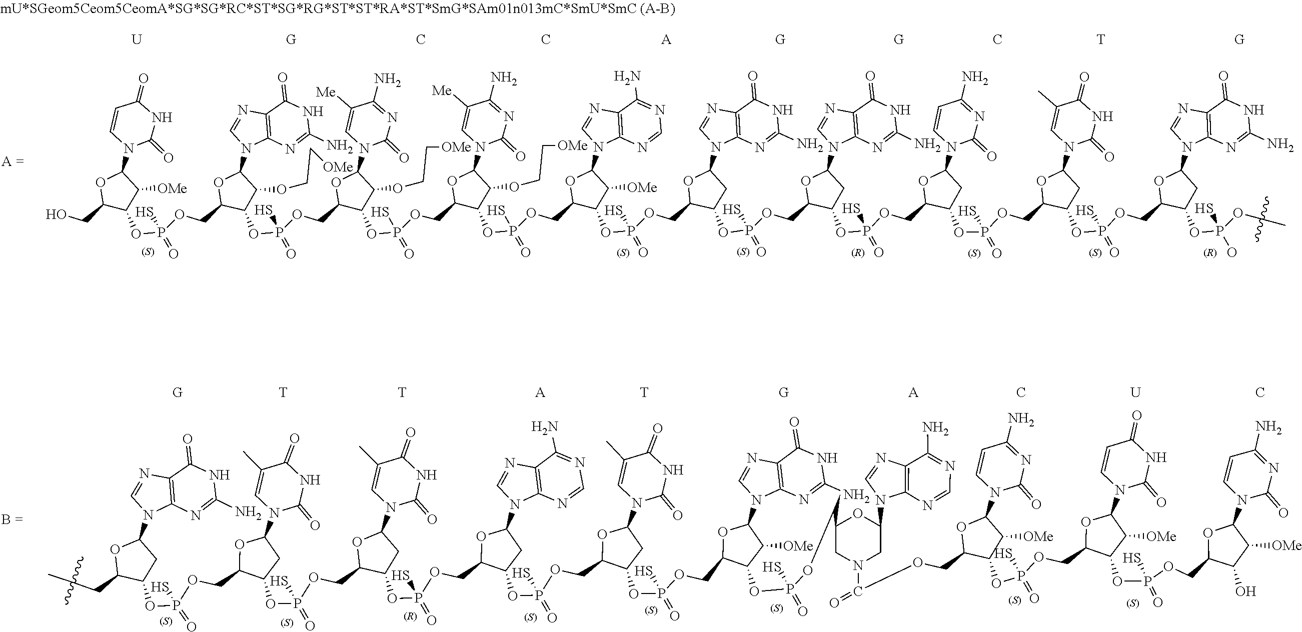

### Production of Oligonucleotides and Compositions

In some embodiments, the present disclosure provides technologies for producing oligonucleotides and compositions as described herein, particularly those comprising sugars comprising nitrogen and/or acyclic sugars as described herein. Among other things, Applicant recognizes that presence of certain structural feature, e.g., sugars comprising nitrogen and/or acyclic sugars and related internucleotidic linkages, typically in combination with other types of sugars and internucleotidic linkages, can present significant production challenges; in some embodiments, the present disclosure provides developed technologies to address such challenges for manufacturing various oligonucleotides and compositions of the present disclosure.

For example, in some embodiments, the present disclosure provides technologies (e.g., reagents, methods, intermediates, etc.) for preparing oligonucleotides comprising sugars comprising nitrogen. In some embodiments, such oligonucleotides also comprise one or more ribose sugars each of which is independently and optionally modified. In some embodiments, one or more sugars comprising nitrogen independently comprise a ring that comprises a ring nitrogen atom. In some embodiments, one or more sugars comprising nitrogen independently comprise a ring that comprises a ring nitrogen atom which is bond to an internucleotidic linkage. In some embodiments, one or more sugars comprising nitrogen independently comprise a ring that comprises a ring nitrogen atom which is bond to a linkage phosphorus of an internucleotidic linkage. In some embodiments, one or more sugars comprising nitrogen are independently acyclic sugars. In some embodiments, oligonucleotides comprise one or more sugars each independently comprising a ring that comprises a ring nitrogen atom which is bond to a linkage phosphorus of an internucleotidic linkage, and one or more optionally modified ribose sugars. In some embodiments, a provide method comprises a coupling step that comprises:

contacting a first compound with a second compound in the presence of a base.

In various embodiments, a first compound is a coupling partner compound as described herein. In some embodiments, a second compound comprises a suitable reactive group, e.g., a hydroxyl or an amino group. In some embodiments, a second compound is a nucleoside (in many embodiments, a nucleoside in an oligonucleotide) comprising a suitable reactive group, e.g., a hydroxyl, an amino group, etc. In some embodiments, a nucleoside comprises —OH. In some embodiments, a nucleoside comprises —NHR. In some embodiments, a nucleoside comprises —NH₂. In some embodiments, a nucleoside is connected to a support, e.g., a solid support like CPG. In some embodiments, a nucleoside is of an oligonucleotide. In some embodiments, an oligonucleotide is connected to a support, e.g., a solid support like CPG suitable for oligonucleotide synthesis. In some embodiments, a nucleoside is a 5′-end nucleoside of an oligonucleotide. As appreciated by those skilled in the art, a coupling step may be utilized in synthesis cycles for preparing oligomers or polymers such as oligonucleotides. Typically, a coupling step forms a linkage between a first and a second compound which has the structure of an internucleotidic linkage as described herein (though may not necessary be an internucleotidic linkage when the linkage is not connecting two nucleosides).

In some embodiments, a cycle comprises a coupling step, a capping step, and a deprotection step. In some embodiments, a cycle consists of a coupling step, a capping step, and a deprotection step. In some embodiments, each step may be independently repeated, and may comprise various procedures such as contacting, incubating, washing, etc. In some embodiments, a cycle may further comprise a modification step (e.g., installing a moiety on linkage phosphorus such as ═O, ═S, ═N—, etc.). In some embodiments, a cycle comprises no modification steps that directly modify linkage phosphorus atoms.

### Coupling Partner Compounds

In some embodiments, the present disclosure provides various compounds that, among other things, can be utilized to prepare oligonucleotides. In some embodiments, they can be utilized to be coupled to nucleosides and/or oligonucleotides to extend oligonucleotide chains.

In some embodiments, a compound comprises a structure of

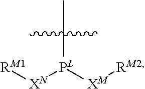

wherein each variable is independently as described herein. In some embodiments, X^{N} is —O—. In some embodiments, a compound comprises a structure of

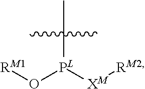

wherein each variable is independently as described herein. In some embodiments, P^{L} is bonded to an oxygen atom in addition to the X^{M} and X^{N}. In some embodiments, P^{L} is bonded to a nitrogen atom in addition to the X^{M} and X^{N}. In some embodiments, a compound has the structure of formula M-I:

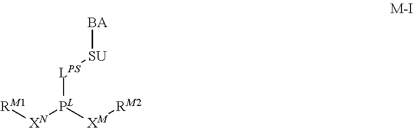

or a salt thereof, wherein:

each of X^{M} and X^{N} is independently -L-O—, -L-S— or -L-NR^{MN}—;

P^{L} is P, P(═W), P->B(-L^{L}-R^{L})₃, or P^{N};

W is O, N(-L^{L}-R^{L}), S or Se;

P^{N} is P═N—C(-L^{L}-R′)(=L^{N}-R′) or P═N-L^{L}-R^{L};

L^{N} is =N-L^{L1}-, ═CH-L^{L1}- wherein CH is optionally substituted, or ═N⁺(R′)(Q⁻)-L^{L1}-;

each L^{L1} is independently L;

Q⁻ is an anion;

each of R^{M1}, R^{M2} and R^{MN} is independently -L^{M}-R^{M};

each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

each R^{L} is independently -L^{L}-R′ or —N═C(-L^{L}-R′)₂;

each of L^{L} and L^{M} is independently L;

BA is a nucleobase;

SU is a sugar;

L^{PS} is a L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted, 3-30 membered, monocyclic, bicyclic or polycyclic ring having 0-10 heteroatoms;

each Cy^{L} is independently an optionally substituted, trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, X^{M} is —S— or —NR^{MN}—. In some embodiments, X^{M} is —S—. In some embodiments, X^{M} is —NR^{MN}—. In some embodiments, X^{N} is —O— or —S—. In some embodiments, X^{N} is —O—. In some embodiments, X^{N} is —S—. In some embodiments, a compound of formula M-I has the structure of

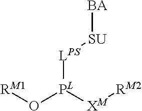

or a salt thereof.

In some embodiments, as described in the present disclosure, two (e.g., R^{M1} and R^{M2}) or more (e.g., R^{M1}, R^{M2} and R^{MN}) groups each of which can be R are taken together with their intervening atoms to form a ring, e.g., an optionally substituted, 3-30 membered, monocyclic, bicyclic or polycyclic ring having, in addition to the intervening atoms, 0-10 heteroatoms. In some embodiments, R^{M1} and R^{M2} are taken together with their intervening atoms to form an optionally substituted, 3-30 membered, monocyclic, bicyclic or polycyclic ring having, in addition to the intervening atoms, 0-10 heteroatoms. In some embodiments, R^{M1}, R^{M2} and R^{MN} are taken together with their intervening atoms to form an optionally substituted, 3-30 membered, monocyclic, bicyclic or polycyclic ring having, in addition to the intervening atoms, 0-10 heteroatoms. In some embodiments, a formed ring is 3-20, 3-15, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, it is substituted. In some embodiments, it is unsubstituted (except for moieties connected to intervening atoms). In some embodiments, it is monocyclic. In some embodiments, it is bicyclic. In some embodiments, it is polycyclic. In some embodiments, it has no additional heteroatoms in addition to the intervening atoms. In some embodiments, when a ring is bicyclic or polycyclic, each monocyclic ring (e.g., each of the two monocyclic rings of a bicyclic ring) is independently an optionally substituted 3-10 membered ring having 0-10 heteroatoms. In some embodiments, a monocyclic ring is saturated. In some embodiments, each monocyclic ring is saturated. In some embodiments, a monocyclic ring is partially unsaturated. In some embodiments, each monocyclic ring is partially unsaturated. In some embodiments, a monocyclic ring is aromatic. In some embodiments, each monocyclic ring is aromatic. In some embodiments, a monocyclic ring is cycloaliphatic. In some embodiments, each monocyclic ring is cycloaliphatic. In some embodiments, a monocyclic ring is heterocyclyl. In some embodiments, each monocyclic ring is heterocyclyl. In some embodiments, each monocyclic ring is aromatic. In some embodiments, a monocyclic ring is aryl. In some embodiments, each monocyclic ring is aryl. In some embodiments, a monocyclic ring is heteroaryl. In some embodiments, each monocyclic ring is heteroaryl. In some embodiments, at least one monocyclic ring is aromatic, and at least one monocyclic ring is partially unsaturated. In some embodiments, at least one monocyclic ring is saturated, and at least one monocyclic ring is partially unsaturated. In some embodiments, at least one monocyclic ring is aromatic, and at least one monocyclic ring is saturated.

In some embodiments,

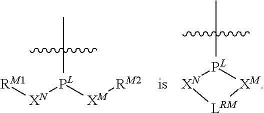

In some embodiments,

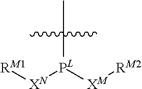

is optionally substituted

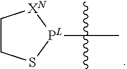

In some embodiments,

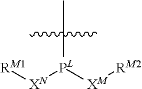

is optionally substituted

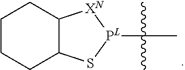

In some embodiments, X^{N} is O. In some embodiments, X^{N} is S. In some embodiments, —X^{N}—R^{M1} is —O—CH₂—CH₂—CN. In some embodiments, —X^{M}—R^{M2} is —N(R)₂. In some embodiments, —X^{M}—R^{M2} is —N(i-Pr)₂.

In some embodiments,

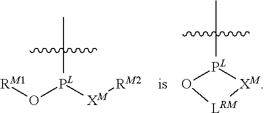

In some embodiments,

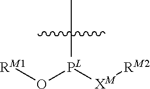

is optionally substituted

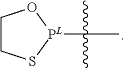

In some embodiments,

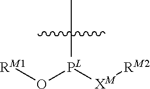

is optionally substituted

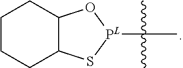

In some embodiments,

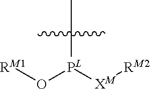

is optionally substituted

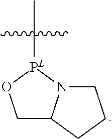

In some embodiments, a compound comprises a structure of

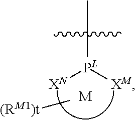

wherein each variable is independently as described herein. In some embodiments, X^{N} is O. In some embodiments, a compound comprises a structure of

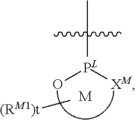

wherein each variable is independently as described herein. In some embodiments, P^{L} is bonded to an oxygen atom in addition to the X^{N} and X^{M}. In some embodiments, P^{L} is bonded to a nitrogen atom in addition to the X^{N} and X^{M}. In some embodiments, a compound has the structure of formula M-II:

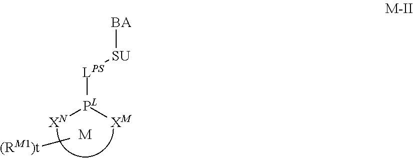

or a salt thereof, wherein:

each of X^{M} and X^{N} is independently -L-O—, -L-S— or -L-NR^{MN}—;

P^{L} is P, P(═W), P->B(-L^{L}-R^{L})₃, or P^{N};

W is O, S or Se;

P^{N} is P═N—C(-L^{L}-R′) or P═N-L^{L}-R^{L};

L^{N} is ═N-L^{L1}-, ═CH-L^{L1}- wherein CH is optionally substituted, or ═M⁺(R′)(Q⁻)-L^{L1}-;

each L^{L1} is independently L;

Q⁻ is an anion;

each of R^{M1} and R^{MN} is independently -L^{M}-R^{M};

each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

each R^{L} is independently -L^{L}-R′ or —N═C(L^{L}-R′)₂;

t is 0-10;

each of L^{L} and L^{L1} is independently L;

Ring M is an optionally substituted 3-30 membered, monocyclic, bicyclic or polycyclic ring having 0-10 heteroatoms;

BA is a nucleobase;

SU is a sugar;

L^{PS} is L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, 13S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, X^{M} is —S— or —NR^{MN}; —. In some embodiments, X^{M} is —S—. In some embodiments, X^{M} is —NR^{MN}—. In some embodiments, X^{N} is —O— or —S—. In some embodiments, X^{N} is —O—. In some embodiments, X^{N} is —S—. In some embodiments, a compound of formula M-II has the structure of

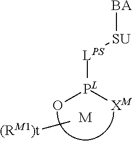

or a salt thereof.

In some embodiments,

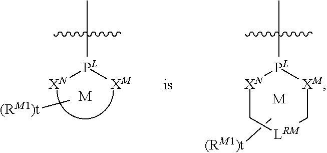

wherein L^{RM} is L′; L′ is a covalent bond, or a bivalent, optionally substituted, linear or branched group selected from a C₁₋₂₀ aliphatic group and a C₁₋₂₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}, and each other variable is independently as described herein. In some embodiments,

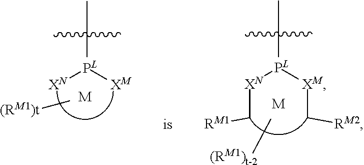

wherein each of R^{M1′} is independently R^{M}, and each other variable is as described herein. In some embodiments,

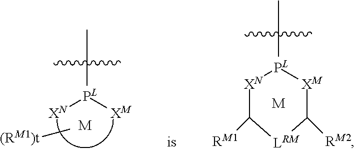

wherein each variable is independently as described herein. In some embodiments, L^{RM} is optionally substituted —CH₂—. In some embodiments, L^{RM} is —CH₂—. In some embodiments, L^{RM} is a covalent bond. In some embodiments,

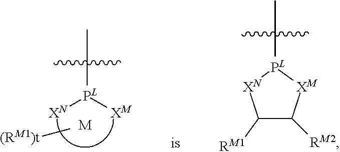

wherein each variable is independently as described herein. In some embodiments,

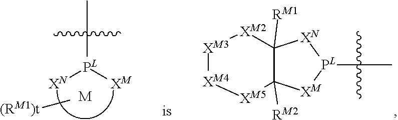

wherein each of X^{M2}, X^{M3}, X^{M4}, and X^{M5} is independently a covalent bond, optionally substituted —CH₂—or —C(R^{M})₂—, and each other variable is independently as described herein. In some embodiments,

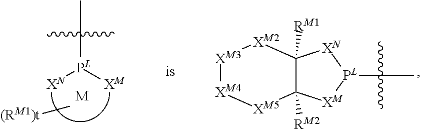

wherein each variable is independently as described herein. In some embodiments,

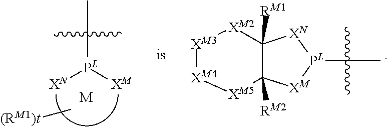

wherein each variable is independently as described herein. In some embodiments,

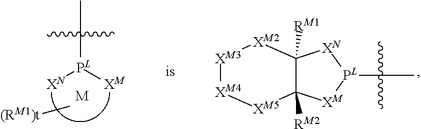

wherein each variable is independently as described herein. In some embodiments,

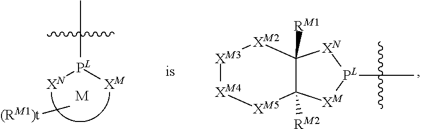

wherein each variable is independently as described herein. In some embodiments, X^{N} is O. In some embodiments, X^{N} is S.

In some embodiments,

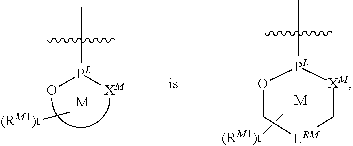

wherein each variable is independently as described herein. In some embodiments,

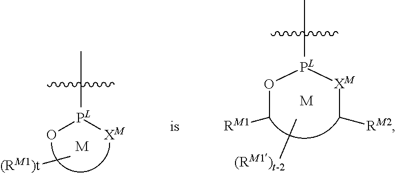

wherein each of R^{M1′} is independently R^{M}, and each other variable is as described herein. In some embodiments,

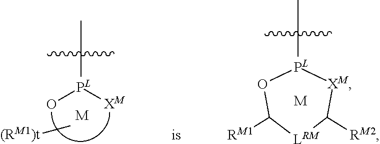

wherein each variable is independently as described herein. In some embodiments, L^{RM} is optionally substituted —CH₂—. In some embodiments, L^{RM} is —CH₂—. In some embodiments, L^{RM} is a covalent bond. In some embodiments,

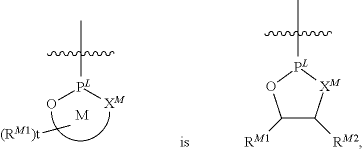

wherein each variable is independently as described herein. In some embodiments,

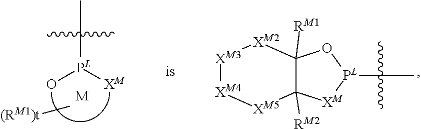

wherein each of X^{M2}, X^{M3}, X^{M4}, and X^{M5} is independently a covalent bond, optionally substituted —CH₂—or —C(R^{M})₂—, and each other variable is independently as described herein. In some embodiments,

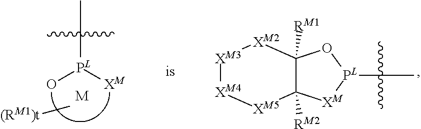

wherein each variable is independently as described herein. In some embodiments,

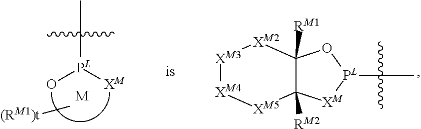

wherein each variable is independently as described herein. In some embodiments,

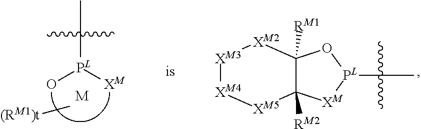

wherein each variable is independently as described herein. In some embodiments,

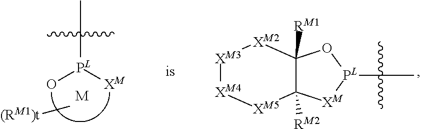

wherein each variable is independently as described herein.

In some embodiments,

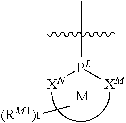

is optionally substituted

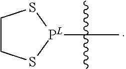

In some embodiments,

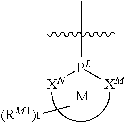

is optionally substituted

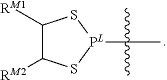

In some embodiments,

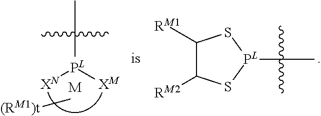

In some embodiments,

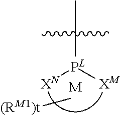

is optionally substituted

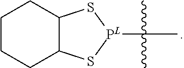

In some embodiments,

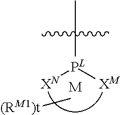

is optionally substituted

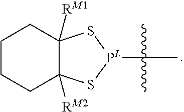

In some embodiments,

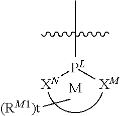

is optionally substituted

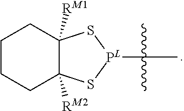

In some embodiments,

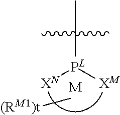

is optionally substituted

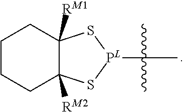

In some embodiments,

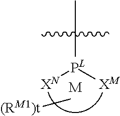

is optionally substituted

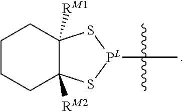

In some embodiments,

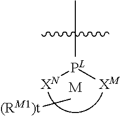

is optionally substituted

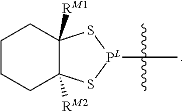

In some embodiments,

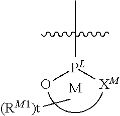

is optionally substituted

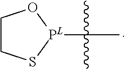

In some embodiments,

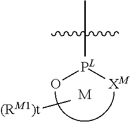

is optionally substituted

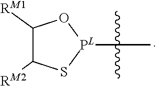

In some embodiments,

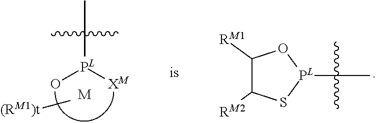

In some embodiments,

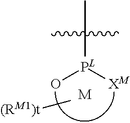

is optionally substituted

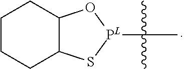

In some embodiments,

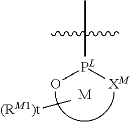

is optionally substituted

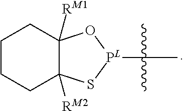

In some embodiments,

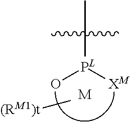

is optionally substituted

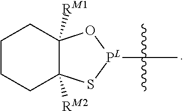

In some embodiments,

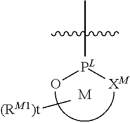

is optionally substituted

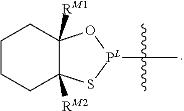

In some embodiments is

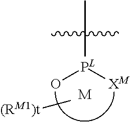

optionally substituted

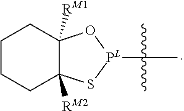

In some embodiments,

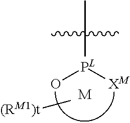

is optionally substituted

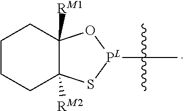

In some embodiments,

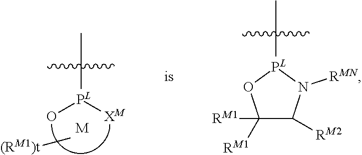

wherein each variable is independently as described herein. In some embodiments, each R^{M1} is independently R. In some embodiments, one R^{M1} is hydrogen. In some embodiments,

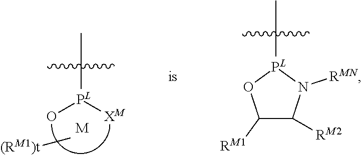

wherein each variable is independently as described herein. In some embodiments, R^{M2} and R^{MN} are taken together to form a ring as described herein. In some embodiments, a formed ring is an optionally substituted 3-30 membered ring having 0-10 heteroatoms in addition to the nitrogen. In some embodiments, a formed ring is an optionally substituted 3-10 membered saturated or partially unsaturated monocyclic ring. In some embodiments, a formed ring is an optionally substituted 3-10 membered monocyclic saturated ring. In some embodiments, a formed ring is 4-membered. In some embodiments, a formed ring is 5-membered. In some embodiments, a formed ring is 6-membered. In some embodiments, a formed ring having no heteroatoms in addition to the nitrogen. In some embodiments, a formed ring is an optionally substituted 5-membered saturated ring having no heteroatoms in addition to nitrogen. In some embodiments, R^{M1} and R^{M2} are cis. In some embodiments, In some embodiments,

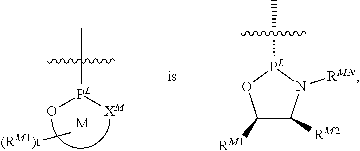

wherein R^{M2} and R^{MN} are taken together to form a ring as described herein. In some embodiments,

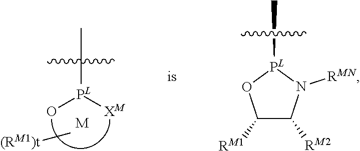

wherein R^{M2} and R^{MN} are taken together to form a ring as described herein. In some embodiments,

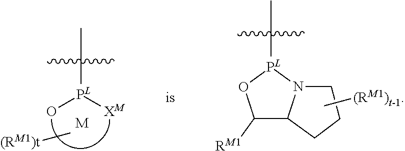

In some embodiments,

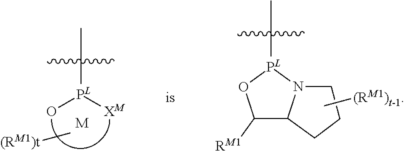

In some embodiments,

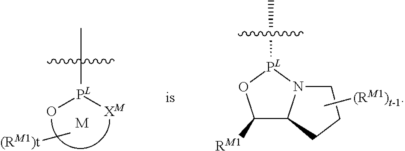

In some embodiments,

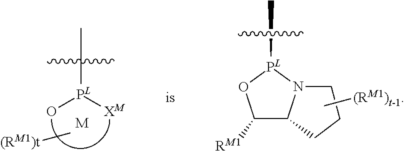

In some embodiments,

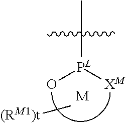

is optionally substituted

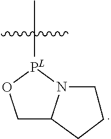

In some embodiments,

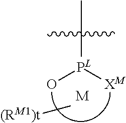

is optionally substituted

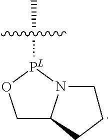

In some embodiments,

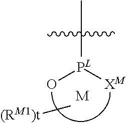

is optionally substituted

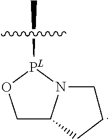

In some embodiments, R^{M1} is —CH₂—Si(R)₃, wherein the —CH₂— is optionally substituted, and each R is not hydrogen. In some embodiments, R^{M1} is —CH₂—SiPh₂Me. In some embodiments, R^{M1} comprises an electron-withdrawing group, for example, in some embodiments, R^{M1} is —CH₂—SO₂R, wherein the —CH₂— is optionally substituted. In some embodiments, R^{M1} is —CH₂—SO₂R, wherein R is as described herein and is not —H. In some embodiments, R is optionally substituted C₁₋₆ aliphatic. In some embodiments, R is optionally substituted C₁₋₆ alkyl. In some embodiments, R is optionally substituted phenyl. In some embodiments, R is phenyl.

In some embodiments,

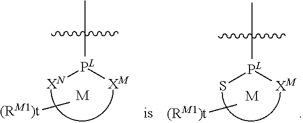

In some embodiments,

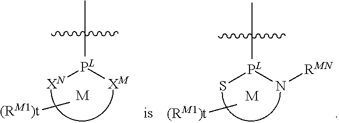

In some embodiments,

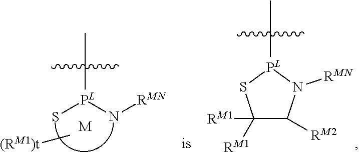

wherein each variable is independently as described herein. In some embodiments, each R^{M1} is independently R. In some embodiments, one R^{M1} is hydrogen. In some embodiments,

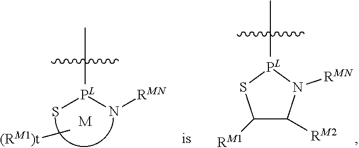

wherein each variable is independently as described herein. In some embodiments, R^{M2} and R^{MN} are taken together to form a ring as described herein. In some embodiments, a formed ring is an optionally substituted 3-30 membered ring having 0-10 heteroatoms in addition to the nitrogen. In some embodiments, a formed ring is an optionally substituted 3-10 membered saturated or partially unsaturated monocyclic ring. In some embodiments, a formed ring is an optionally substituted 3-10 membered monocyclic saturated ring. In some embodiments, a formed ring is 4-membered. In some embodiments, a formed ring is 5-membered. In some embodiments, a formed ring is 6-membered. In some embodiments, a formed ring having no heteroatoms in addition to the nitrogen. In some embodiments, a formed ring is an optionally substituted 5-membered saturated ring having no heteroatoms in addition to nitrogen. In some embodiments, R^{M1} and R^{M2} are cis. In some embodiments, In some embodiments,

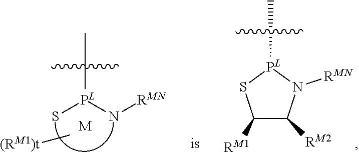

wherein R^{M2} and R^{MN} are taken together to form a ring as described herein. In some embodiments,

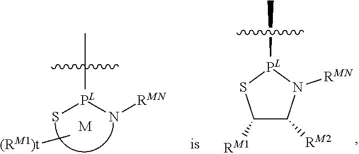

wherein R^{M2} and R^{MN} are taken together to form a ring as described herein. In some embodiments,

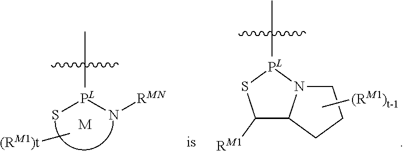

In some embodiments,

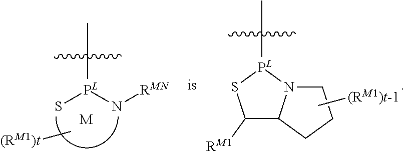

In some embodiments,

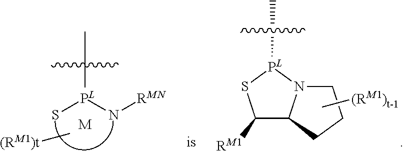

In some embodiments,

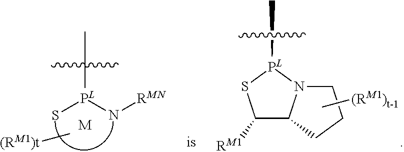

In some embodiments,

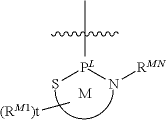

is optionally substituted

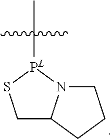

In some embodiments,

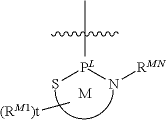

is optionally substituted

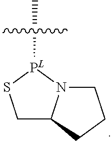

In some embodiments,

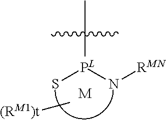

is optionally substituted

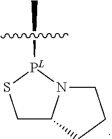

In some embodiments, R^{M1} is —CH₂—Si(R)₃, wherein the —CH₂— is optionally substituted, and each R is not hydrogen. In some embodiments, R^{M1} is —CH₂—SiPh₂Me. In some embodiments, R^{M1} comprises an electron-withdrawing group, for example, in some embodiments, R^{M1} is —CH₂—SO₂R, wherein the —CH₂— is optionally substituted. In some embodiments, R^{M1} is —CH₂—SO₂R, wherein R is as described herein and is not —H. In some embodiments, R is optionally substituted C₁₋₆ aliphatic. In some embodiments, R is optionally substituted C₁₋₆ alkyl. In some embodiments, R is optionally substituted phenyl. In some embodiments, R is phenyl.

n some embodiments, R^{M1} and R^{M2} are cis. In some embodiments, R^{M1} and R^{M2} are trans. In some embodiments, R^{M1} is cis to the addition moiety bonded to P^{L} (other than the O and S). In some embodiments, each of R^{M1} and R^{M2} is independently R. In some embodiments, R^{M1} is optionally substituted C₁₋₆ aliphatic. In some embodiments, R^{M2} is optionally substituted C₁₋₆ aliphatic. In some embodiments, each of R^{M1} and R^{M2} is independently optionally substituted C₁₋₆ aliphatic. In some embodiments, R^{M1} is optionally substituted C₁₋₆ alkyl. In some embodiments, R^{M2} is optionally substituted C₁₋₆ alkyl. In some embodiments, each of R^{M1} and R^{M2} is independently optionally substituted C₁₋₆ alkyl. In some embodiments, R^{M1} is methyl. In some embodiments, R^{M2} is methyl. In some embodiments, R^{M1} is —H. In some embodiments, R^{M2} is —H. In some embodiments, both R^{M1} and R^{M2} are —H. In some embodiments, R^{M1} is —H and R^{M2} is not —H. In some embodiments, R^{M1} is not —H and R^{M2} is —H. In some embodiments, neither of R^{M1} and R^{M2} is —H. In some embodiments, R^{M2} is —H and R^{M2} is —CH₃.

In some embodiments, each of X^{M2}, X^{M3}, X^{M4} and X^{M5} is independently optionally substituted —CH₂—. In some embodiments, each of X^{M2}, X^{M3}, X^{M4} and X^{M5} is —CH₂—.

In some embodiments, X^{M3} is —CHR—. In some embodiments, R is optionally substituted C₁₋₆ aliphatic. In some embodiments, R is cis to R^{M1}. In some embodiments, R is trans to R. In some embodiments, R is —C(CH₃)═CH₂. In some embodiments, R is —CH(CH₃)₂. In some embodiments, each of X^{M2}, X^{M4} and X^{M5} is —CH₂—. In some embodiments, R^{M1} is —H and R^{M2} is optionally substituted C₁₋₆ aliphatic. In some embodiments, R^{M1} is —H and R^{M2} is methyl. In some embodiments, R^{M2} is —H and R^{M1} is optionally substituted C₁₋₆ aliphatic. In some embodiments, R^{M2} is —H and R^{M1} is methyl. In some embodiments, both R^{M1} and R^{M2} are independently optionally substituted C₁₋₆ aliphatic. In some embodiments, both R^{M1} and R^{M2} are methyl.

In some embodiments, X^{M4} is —CHR—. In some embodiments, R is optionally substituted C₁₋₆ aliphatic. In some embodiments, R is trans to R^{M1}. In some embodiments, R is cis to R. In some embodiments, R is —C(CH₃)═CH₂. In some embodiments, R is —CH(CH₃)₂. In some embodiments, each of X^{M2}, X^{M3} and X^{M5} is —CH₂—. In some embodiments, R^{M1} is —H and R^{M2} is optionally substituted C₁₋₆ aliphatic. In some embodiments, R^{M1} is —H and R^{M2} is methyl. In some embodiments, R^{M2} is —H and R^{M1} is optionally substituted C₁₋₆ aliphatic. In some embodiments, R^{M2} is —H and R^{M1} is methyl. In some embodiments, both R^{M1} and R^{M2} are independently optionally substituted C₁₋₆ aliphatic. In some embodiments, both R^{M1} and R^{M2} are methyl.

In some embodiments, X^{M2} is —C(R)₂—, and X^{M5} is —C(R)₂—. In some embodiments, X^{M2} is —C(R)₂—, and X^{M5} is —CHR—. In some embodiments, one R of X^{M2} and one R of X^{M5} are taken together to form -L^{XM}-, wherein L^{XM} is an optionally substituted bivalent C₁₋₄ aliphatic or heteroaliphatic having 1-4 heteroatoms. In some embodiments, C^{M} is optionally substituted —CH₂—. In some embodiments, C^{M} is —C(CH₃)₂—. In some embodiments, the one R of X^{M2} and one R of X^{M5} are cis. In some embodiments, the one R of X^{M2} and one R of X^{M5} are cis, and are trans to R^{M1}. In some embodiments, the one R of X^{M2} and one R of X^{M5} are cis, and are cis to R^{M1}. In some embodiments, the other R of X^{M2} is —H. In some embodiments, the other R of X^{M2} is C₁₋₆ aliphatic. In some embodiments, the other R of X^{M2} is methyl. In some embodiments, each of X^{M3} and X^{M4} is —CH₂—. In some embodiments, R^{M1} and R^{M2} are cis. In some embodiments, R^{M1} and R^{M2} are trans. In some embodiments, both of R^{M1} and R^{M2} are —H. In some embodiments, R^{M1} is —H and R^{M2} is optionally substituted C₁₋₆ aliphatic. In some embodiments, R^{M1} is —H and R^{M2} is methyl. In some embodiments, R^{M2} is —H and R^{M1} is optionally substituted C₁₋₆ aliphatic. In some embodiments, R^{M2} is —H and R^{M1} is methyl. In some embodiments, both R^{M1} and R^{M2} are independently optionally substituted C₁₋₆ aliphatic. In some embodiments, both R^{M1} and R^{M2} are methyl.

In some embodiments, X^{M2} is —C(R)₂—, and X^{M5} is —C(R)₂—. In some embodiments, X^{M5} is —C(R)₂—, and X^{M2} is —CHR—. In some embodiments, one R of X^{M2} and one R of X^{M5} are taken together to form -L^{XM}-, wherein L^{XM} is an optionally substituted bivalent C₁₋₄ aliphatic or heteroaliphatic having 1-4 heteroatoms. In some embodiments, L^{XM} is optionally substituted —CH₂—. In some embodiments, L^{XM} is —C(CH₃)₂—. In some embodiments, the one R of X^{M2} and one R of X^{M5} are cis. In some embodiments, the one R of X^{M2} and one R of X^{M5} are cis, and are trans to R^{M1}. In some embodiments, the one R of X^{M2} and one R of X^{M5} are cis, and are cis to R^{M1}. In some embodiments, the other R of X^{M5} is —H. In some embodiments, the other R of X^{M5} is C₁₋₆ aliphatic. In some embodiments, the other R of X^{M5} is methyl. In some embodiments, each of X^{M3} and X^{M4} is —CH₂—. In some embodiments, R^{M1} and R^{M2} are cis. In some embodiments, R^{M1} and R^{M2} are trans. In some embodiments, both of R^{M1} and R^{M2} are —H. In some embodiments, R^{M1} is —H and R^{M2} is optionally substituted C₁₋₆ aliphatic. In some embodiments, R^{M1} is —H and R^{M2} is methyl. In some embodiments, R^{M2} is —H and R^{M1} is optionally substituted C₁₋₆ aliphatic. In some embodiments, R^{M2} is —H and R^{M1} is methyl. In some embodiments, both R^{M1} and R^{M2} are independently optionally substituted C₁₋₆ aliphatic. In some embodiments, both R^{M1} and R^{M2} are methyl.

In some embodiments, X^{M2} is —C(R)₂—, and X^{M4} is —C(R)₂—. In some embodiments, X^{M2} is —C(R)₂—, and X^{M4} is —CHR—. In some embodiments, X^{M4} is —C(R)₂—, and X^{M2} is —CHR—. In some embodiments, X^{M2} is —CHR—, and X^{M4} is —CHR—. In some embodiments, one R of X^{M2} and one R of X^{M}4 are taken together to form -L^{XM}—, wherein L^{XM} is an optionally substituted bivalent C₁₄ aliphatic or heteroaliphatic having 1-4 heteroatoms. In some embodiments, L^{XM} is optionally substituted —CH₂—. In some embodiments, L^{XM} is —C(CH₃)₂—. In some embodiments, the one R of X^{M2} and one R of X^{M4} are cis. In some embodiments, the one R of X^{M2} and one R of X^{M4} are cis, and are trans to R^{M1}. In some embodiments, the one R of X^{M2} and one R of X^{M4} are cis, and are cis to R^{M1}. In some embodiments, the other R of X^{M2} is —H. In some embodiments, the other R of X^{M2} is C₁₋₆ aliphatic. In some embodiments, the other R of X^{M2} is methyl. In some embodiments, the other R of X^{M4} is —H. In some embodiments, the other R of X^{M4} is C₁₋₆ aliphatic. In some embodiments, the other R of X^{M4} is methyl. In some embodiments, each of X^{M3} and X^{M5} is —CH₂—. In some embodiments, R^{M1} and R^{M2} are cis. In some embodiments, R^{M1} and R^{M2} are trans. In some embodiments, both of R^{M1} and R^{M2} are —H. In some embodiments, R^{M1} is —H and R^{M2} is optionally substituted C₁₋₆ aliphatic. In some embodiments, R^{M1} is —H and R^{M2} is methyl. In some embodiments, R^{M2} is —H and R^{M1} is optionally substituted C₁₋₆ aliphatic. In some embodiments, R^{M2} is —H and R^{M1} is methyl. In some embodiments, both R^{M1} and R^{M2} are independently optionally substituted C₁₋₆ aliphatic. In some embodiments, both R^{M1} and R^{M2} are methyl.

In some embodiments, X^{M3} is —C(R)₂—, and X^{M5} is —C(R)₂—. In some embodiments, X^{M3} is —C(R)₂—, and X^{M5} is —CHR—. In some embodiments, X^{M5} is —C(R)₂—, and X^{M3} is —CHR—. In some embodiments, X^{M3} is —CHR—, and X^{M5} is —CHR—. In some embodiments, one R of X³ and one R of X^{M}5 are taken together to form -L^{XM}; wherein L^{XM} is an optionally substituted bivalent C₁₋₄ aliphatic or heteroaliphatic having 1-4 heteroatoms. In some embodiments, L^{XM} is optionally substituted —CH₂—. In some embodiments, L^{XM} is —C(CH₃)₂—. In some embodiments, the one R of X^{M3} and one R of X^{M5} are cis. In some embodiments, the one R of X^{M3} and one R of X^{M5} are cis, and are trans to R^{M1}. In some embodiments, the one R of X^{M3} and one R of X^{M5} are cis, and are cis to R^{M1}. In some embodiments, the other R of X^{M3} is —H. In some embodiments, the other R of X^{M3} is C₁₋₆ aliphatic. In some embodiments, the other R of X^{M3} is methyl. In some embodiments, the other R of X^{M5} is —H. In some embodiments, the other R of X^{M5} is C₁₋₆ aliphatic. In some embodiments, the other R of X^{M5} is methyl. In some embodiments, each of X^{M2} and X^{M4} is —CH₂—. In some embodiments, R^{M1} and R^{M2} are cis. In some embodiments, RMI and R^{M2} are trans. In some embodiments, both of R^{M1} and R^{M2} are —H. In some embodiments, R^{M1} is —H and R^{M2} is optionally substituted C₁₋₆ aliphatic. In some embodiments, R^{M1} is —H and R^{M2} is methyl. In some embodiments, R^{M2} is —H and R^{M1} is optionally substituted C₁₋₆ aliphatic. In some embodiments, R^{M2} is —H and R^{M1} is methyl. In some embodiments, both R^{M1} and R^{M2} are independently optionally substituted C₁₋₆ aliphatic. In some embodiments, both R^{M1} and R^{M2} are methyl.

In some embodiments, each of X^{M}2_{, X}M3, X^{M}4 and X^{M5} is independently optionally substituted —CH₂—. In some embodiments, each of X^{M2}, X^{M3}, X^{M4} and X^{M5} is —CH₂—. In some embodiments, one of X^{M2}, X^{M3}, X^{M4} and X^{M5} is a covalent bond. In some embodiments, two or more of X^{M2}, X^{M3}, X^{M4} _{and} X^{M5} are each a covalent bond. In some embodiments, X^{M2} is a covalent bond. In some embodiments, X^{M3} is a covalent bond. In some embodiments, X^{M4} is a covalent bond. In some embodiments, X^{M5} is a covalent bond. In some embodiments, one of X^{M2}, X^{M3}, X^{M4} and X^{M5} is a covalent bond, and each of the others is independently optionally substituted —CH₂—. In some embodiments, one of X^{M2}, X^{M3}, X^{M4} and X^{M5} is a covalent bond, and each of the others is independently —CH₂—. In some embodiments, two or more of X^{M}2, X^{M3}, X^{M4} and X^{M5} are independently —C(R)₂—. In some embodiments, two of X^{M2}, X^{M3}, X^{M4} and X^{M5} are independently —CHR—. In some embodiments, two R groups of two of X^{M2}, X^{M3}, X^{M4} and X^{M5} are taken together to form a ring as described herein. For example, in some embodiments, X^{M4} and X^{M5} are independently —CHR—, and the two R groups are taken together with their intervening atoms to form an optionally substituted phenyl ring.

In some embodiments,

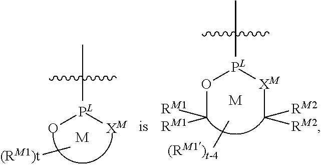

wherein each variable is as described herein. In some embodiments,

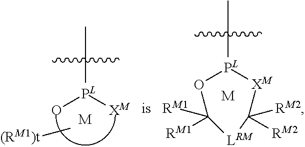

wherein each variable
is independently as described herein. In some embodiments,

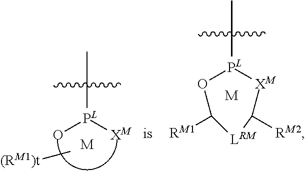

wherein each variable is independently as described herein. In some embodiments, L^{RM} is optionally substituted —CH₂—. In some embodiments, L^{RM} is —CH₂—. In some embodiments, L^{RM} is a covalent bond. In some embodiments,

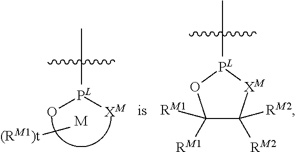

wherein each variable is independently as described herein. In some embodiments,

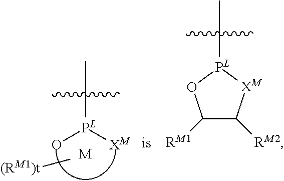

wherein each variable is independently as described herein.

In some embodiments,

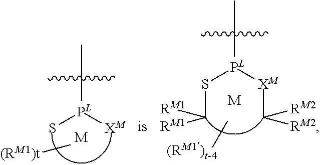

wherein each variable is as described herein. In some embodiments,

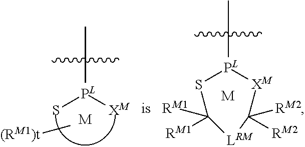

wherein each variable is independently as described herein. In some embodiments,

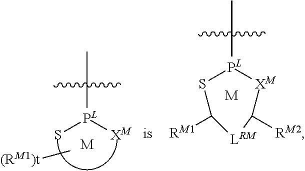

wherein each variable is independently as described herein. In some embodiments, L^{RM} is optionally substituted —CH₂—. In some embodiments, L^{RM} is —CH₂—. In some embodiments, L^{RM} is a covalent bond. In some embodiments,

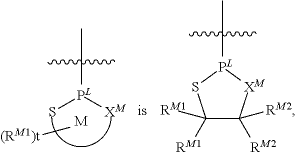

wherein each variable is independently as described herein. In some embodiments,

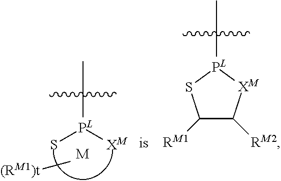

wherein each variable is independently as described herein.

As described herein, in some embodiments, R^{M1} and R^{M2} are cis. In some embodiments, R^{M1} and R^{M2} are trans. In some embodiments, both of R^{M1} and R^{M2} are —H. In some embodiments, R^{M1} is —H and R^{M2} is optionally substituted C₁₋₆ aliphatic. In some embodiments, R^{M1} is —H and R^{M2} is methyl. In some embodiments, R^{M2} is —H and R^{M1} is optionally substituted C₁₋₆ aliphatic. In some embodiments, R^{M2} is —H and R^{M1} is methyl. In some embodiments, both R^{M1} and R^{M2} are independently optionally substituted C₁₋₆ aliphatic. In some embodiments, both R^{M1} and R^{M2} are methyl. In some embodiments, one or each of R^{M1} is independently optionally substituted phenyl. In some embodiments, one or each of R^{M1} is independently phenyl. In some embodiments, each of R^{M1} is phenyl. In some embodiments, one or R^{M2} is —H and the other is not —H. In some embodiments, one of R^{M2} is —H and the other is optionally substituted C₁₋₆ aliphatic. In some embodiments, a C₁₋₆ aliphatic group is isopropyl.

In some embodiments, X^{M} is —CH₂—S—, wherein the —CH₂— is optionally substituted. In some embodiments, X^{M} is —CH₂—S—. In some embodiments, one of X^{M2}, X^{M3}, X^{M4} and X^{M5} is independently —C(R)₂—. In some embodiments, R^{M2} and one R of the —C(R)₂—are taken together to form -L^{XM}- as described herein. In some embodiments, L^{XM} is optionally substituted —CH₂—. In some embodiments, L^{XM} is —C(CH₃)₂—. In some embodiments, R^{M2} and the R are cis. In some embodiments, the other R is —H. In some embodiments, the other R is not —H. In some embodiments, the other R is optionally substituted C₁₋₆ aliphatic. In some embodiments, the other R is optionally substituted C₁₋₆ alkyl. In some embodiments, the other R is methyl.

In some embodiments, HO-L^{RM}-X^{M}—H is an auxiliary compound as described herein (e.g., a compound of formula AC-I (e.g., a compound of AC-I-a, AC-I-b, AC-I-c, AC-I-d, or AC-I-e) or a salt thereof or a salt thereof). In some embodiments,

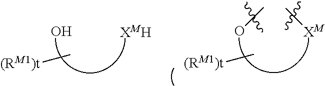

is as in formula M-II, wherein the O and X^{M} are both bonded to P^{L}) is an auxiliary compound as described herein (e.g., a compound of formula AC-I (e.g., a compound of AC-I-a, AC-I-b, AC-I-c, AC-I-d, or AC-I-e) or a salt thereof). In some embodiments, HO-L^{RM}-X^{M}—H or

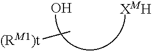

is an auxiliary compound as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774,a WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the chiral auxiliaries and reagents of each of which are incorporated herein by reference.

In some embodiments, P^{L} is P. In some embodiments, P^{L} is P═S. In some embodiments, P^{L} is P═O. In some embodiments, P^{L} is P^{N}. In some embodiments, P^{N} is P═N-L^{L}-R^{L}. In some embodiments, P^{N} is P═N—C(-L^{L}-R′)(=L^{N}-R′). In some embodiments, L^{N} is ═N⁺(R′)(Q⁻)-L^{L1}-. In some embodiments, P^{N} is ═N—C(═N⁺(R′)₂)(N(R′)₂)Q⁻, wherein each variable is independently as described herein. In some embodiments, Q is PF₆⁻. In some embodiments, ═N—C(═N⁺(R′)₂)(N(R′)₂) is

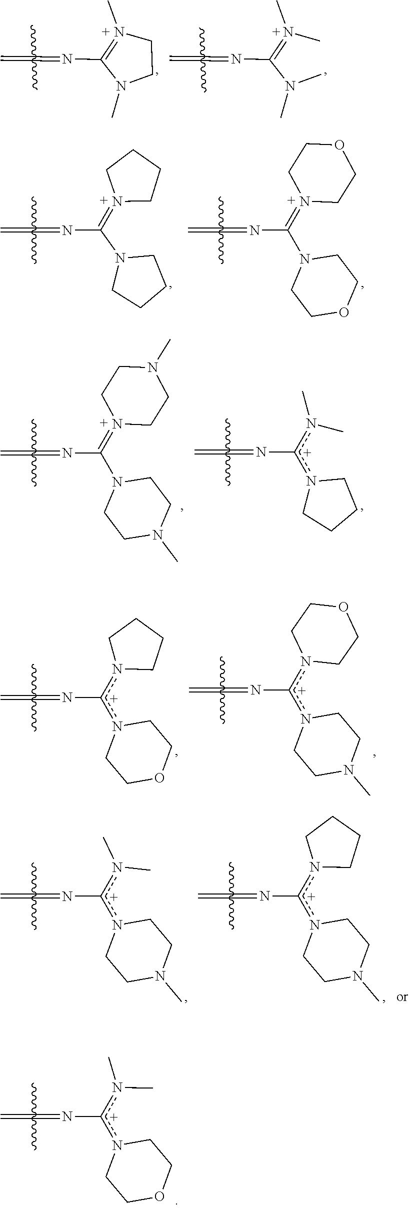

In some embodiments, P^{N} is ═N—SO₂R′. In some embodiments, P^{N} is ═N—C(O)R′. In some embodiments, such P^{N} compounds are prepared by installing the ═N— moiety on P by contacting with a compound comprising —N₃ (e.g., ADIH for

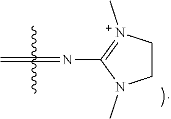 [SMILES: *=NC1=[N+](C)CCN1C]

In some embodiments, a compound is N₃—C(═N⁺(R′)₂)(N(R′)₂)Q⁻. In some embodiments, a compound is N₃—C(-L^{L}-R′)(=L^{N}-R′) or a salt thereof. In some embodiments, a compound is N₃—C(═N⁺(R′)₂)(N(R′)₂)Q⁻). In some embodiments, a compound is N₃—SO₂R′ or a salt thereof. In some embodiments, a compound is N₃—C(O)R′ or a salt thereof.

In some embodiments, a compound has the structure of formula M-III:

BA-SU-C(O)-LG^{M}, M-III

or a salt thereof, wherein:

BA is a nucleobase;

SU is a sugar; and

LG^{M} is a leaving group.

In some embodiments, a leaving group, e.g., LG^{M} is halogen. In some embodiments, a leaving group is —Cl. In some embodiments, LG^{M} is optionally substituted heteroaryl, wherein LG^{M} is bonded to —C(O)— through a nitrogen. In some embodiments, LG^{M} is optionally substituted

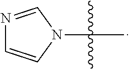 [SMILES: *n1ccnc1]

In some embodiments, LG^{M} is optionally substituted

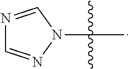 [SMILES: *n1cncn1]

In some embodiments, LG^{M} is

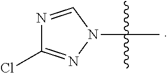 [SMILES: *n1cnc(Cl)n1]

In some embodiments, LG^{M} is

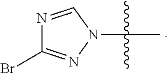 [SMILES: *n1cnc(Br)n1]

In some embodiments, LG^{M} is

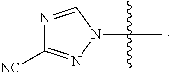 [SMILES: *n1cnc(C#N)n1]

In some embodiments, LG^{M} is

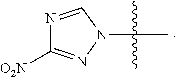 [SMILES: *n1cnc([N+](=O)[O-])n1]

In some embodiments, LG^{M} is —OSu. In some embodiments, —C(O)-LG^{M} is activated carboxylic acid group, e.g., suitable for amidation.

BA and SU are independently as described herein. In some embodiments, BA is a nucleobase as described herein. In some embodiments, BA is or comprises an optionally substituted heteroaryl or heterocyclyl ring. In some embodiments, BA is or comprises a cycloaliphatic ring. In some embodiments, BA comprises a saturated ring. In some embodiments, BA comprises a partially unsaturated ring. In some embodiments, BA comprises an aromatic ring. In some embodiments, BA is optionally substituted A, T, C, or G. In some embodiments, BA is an optionally substituted tautomer of A, T, C, or G. In some embodiments, BA is protected A, T, C or G; particularly, in some embodiments, BA is protected A, T, C, or G suitable of oligonucleotide synthesis.

SU can be a cyclic or acyclic sugar as described herein. In some embodiments, SU is R^{SU}—SU′—, wherein R^{SU} is R^{s}, and —SU′- is a sugar as described herein. For example, in some embodiments, SU′ is

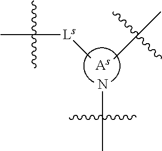

as described herein. In some embodiments, SU′ is sm01. In some embodiments, SU′ is

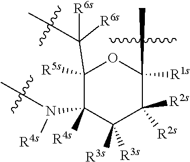

as described herein. In some embodiments, SU′ is

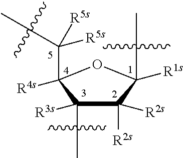

as described herein. In some embodiments, SU′ is

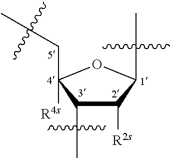

as described herein. In some embodiments, SU′ is

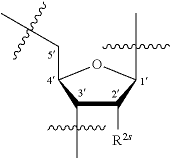

as described herein. In some embodiments, R^{SU} is optionally protected hydroxyl group. In some embodiments, R^{SU} is protected hydroxyl suitable for oligonucleotide synthesis. In some embodiments, R^{SU} is optionally protected amino group. In some embodiments, R^{SU} is —ODMTr.In some embodiments, L^{PS} is a covalent bond, —O—, —S—, or —N(R′)—. In some embodiments, L^{PS} is a covalent bond, —O— or —N(R′)—. In some embodiments, L^{PS} is a covalent bond (e.g., when directly bonded to a sugar nitrogen). In some embodiments, L^{PS} is —O— (e.g., when SU′ is

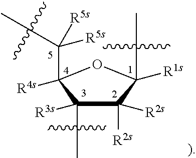

In some embodiments, L^{PS} is —N(R′)—.

In some embodiments, the present disclosure provides technologies for preparing coupling partner compounds, e.g., a compound of formula M-I, M-II, M-III, or a salt thereof.

In some embodiments, the present disclosure provides a method, comprising contacting a compound of formula LG-I:

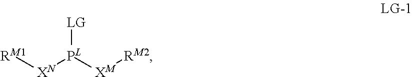

or a salt thereof, wherein LG is a leaving group, and each other variable is independently as described herein,

with a compound having a hydroxyl or amino group.

In some embodiments, the present disclosure provides a method, comprising contacting a compound of formula LG-II:

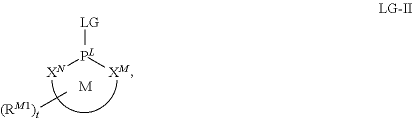

or a salt thereof, wherein LG is a leaving group, and each other variable is independently as described herein,

with a compound having a hydroxyl or amino group.

In some embodiments, a leaving group is halogen. In some embodiments, a leaving group is —Cl. In some embodiments, a leaving group is —N(R)₂, wherein each R is independently an optionally substituted C₁₋₃₀ aliphatic. In some embodiments, each R is isopropyl.

In some embodiments, the present disclosure provides methods for preparing a compound of formula LG-I or LG-II, or a salt thereof, comprising contacting a compound of formula AC-I (e.g., a compound of AC-I-a, AC-I-b, AC-I-c, AC-I-d, or AC-I-e) or a salt thereof with a second compound, e.g., PCl₃.

In some embodiments, P^{L} is P, e.g., in a compound of formula M-I, M-II, LG-I, LG-II, etc.

In some embodiments, a method comprising converting P^{L} which is P (e.g., in a compound of formula M-I or M-II, or a salt thereof) to P^{L} which is P(═W), P->B(-L^{L}-R¹)₃, or P^{N} (e.g., in a compound of formula M-I or M-II, or a salt thereof). In some embodiments, a method comprises converting P to P═S. In some embodiments, a method comprises converting P to P^{N}. In some embodiments, P^{N} is P═N—C(-L^{L}-R′) (=L^{N}-R′), wherein L^{N} is ═N⁺(R′)(Q⁻)-L^{L1}-. In some embodiments, a converting step comprising sulfurization to convert P to P═S. In some embodiments, a converting step comprising converting P to P^{N} utilizing a reagent comprising —N₃ as described herein (e.g., ADIH). In some embodiments, a compound is N₃—C(═N⁺(R′)₂)(N(R′)₂)Q⁻. In some embodiments, a compound is N₃—C(-L^{L}-R′)(=L^{N}-R′) or a salt thereof. In some embodiments, a compound is N₃—C(═N⁺(R′)₂)(N(R′)₂)Q⁻. In some embodiments, a compound is N₃—SO₂R′ or a salt thereof. In some embodiments, a compound is N₃—C(O)R′ or a salt thereof.

In some embodiments, a provided method is useful for preparing a coupling partner compound, e.g., a compound of formula M-I or M-II, or a salt thereof. In some embodiments, a compound has a hydroxyl group. In some embodiments, a compound has an amino group. In some embodiments, a compound is a nucleoside as described herein. In some embodiments, a compound is BA-SU-H as described herein.

In some embodiments, a coupling partner is a short oligonucleotide, e.g., a dimer. Such short oligonucleotide can be prepared and purified (e.g., without using solid support), and then coupled to an oligonucleotide chain using suitable technologies (e.g., coupling through 3′-end nucleoside as if it is a monomeric compound).

As appreciated by those skilled in the art, during preparing one or more chemical groups are independently and optionally protected. Various protection technologies, including many suitable for oligonucleotide synthesis, can be utilized in accordance with the present disclosure. Certain technologies are described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774,a WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the oligonucleotide synthesis technologies, including reagents, protection, conditions (e.g., those for various steps, such as coupling, capping, modifying, deprotection, cleavage, deprotection of bases, removal of auxiliaries, etc.), auxiliaries, cycles, etc. are incorporated herein by reference.

### Auxiliary Compounds

In some embodiments, the present disclosure provides auxiliaries for synthesis, e.g., oligonucleotide preparation. In some embodiments, auxiliaries are chiral auxiliaries, which can facilitate formation of chiral centers, e.g., chiral linkage phosphorus, stereoselectively.

In some embodiments, an auxiliary compound has the structure of formula AC-1:

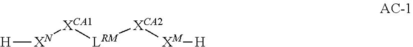

or a salt thereof, wherein:

X^{CA1} is optionally substituted —CH₂—, or —C(R^{M1})(R^{MX1})—;

X^{CA2} is optionally substituted —CH₂—, or —C(R^{M2})(R^{MX2})—;

each of e^{x}' and R^{MX2} is independently R^{M}, or are taken together to form -L^{CA}- or —X^{M2}—X^{M3}—X^{M4}—X^{M5}—;

each of X^{M2}, X^{M3}, X^{M4}, and X^{M5} is independently a covalent bond, optionally substituted —CH₂—or —C(R^{M})₂—,

each of X^{M} and X^{N} is independently -L-O—, -L-S— or -L-NR^{MN}—;

each of R^{M1}, R^{M2} and R^{MN} is independently -L^{M}—R^{M};

each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

each of L^{RM} and L^{CA} is independently L;

BA is a nucleobase;

SU is a sugar;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted, 3-30 membered, monocyclic, bicyclic or polycyclic ring having 0-10 heteroatoms;

each Cy^{L} is independently an optionally substituted, trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments X^{N} is O. In some embodiments X^{N} is S. In cnme embodiments a compound of formula AC-I is a compound of

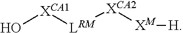

In some embodiments, a compound, e.g. a compound, e.g. a compound of formula AC-I, has the structure of formula AC-I-a:

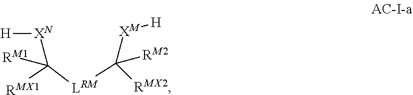

or a salt thereof. In some embodiments, X^{N} is O. In some embodiments, X^{N} is S. In some embodiments, a compound of formula AC-I-a is a compound of

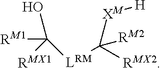

In some embodiments, a compound, e.g. a compound, e.g. a compound of formula AC-I, has the structure of formula AC-I-b:

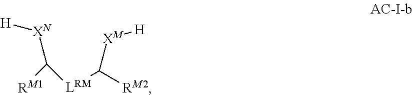

or a salt thereof. In some embodiments, X^{N} is O. In some embodiments, X^{N} is S. In some embodiments, a compound of formula AC-I-b is a compound of

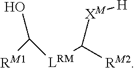

In some embodiments, L^{RM}is a covalent bond.

In some embodiments, a compound, e.g. a compound, e.g. a compound of formula AC-I, has the structure of formula AC-I-c:

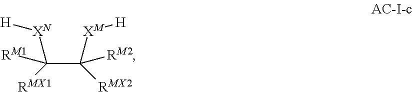

or a salt thereof. In some embodiments, X^{N} is O. In some embodiments, X^{N} is S. In some embodiments, a compound of formula AC-I-b is a compound of

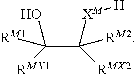

In some embodiments, a compound, e.g. a compound, e.g. a compound of formula AC-I, has the structure of formula AC-I-d:

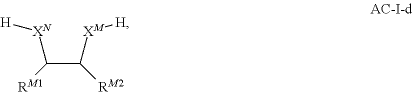

or a salt thereof. In some embodiments, X^{N} is O. In some embodiments, X^{N} is S. In some embodiments, a compound of formula AC-I-b is a compound of

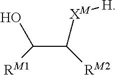

In some embodiments, a compound, e.g. a compound, e.g. a compound of formula AC-I, has the structure of formula AC-I-e:

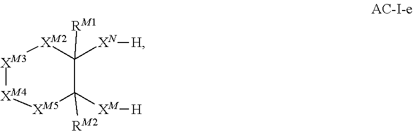

or a salt thereof. In some embodiments, X^{N} is O. In some embodiments, X^{N} is S. In some embodiments, a compound of formula AC-I-b is a compound of

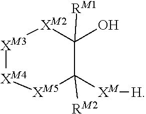

In some embodiments, a compound of formula AC-I-b is a compound of

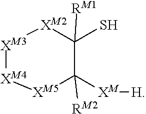

In some embodiments, a compound has the structure of

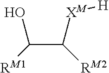

or a salt thereof, wherein each variable is independently as described herein. In some embodiments, a compound has the structure of

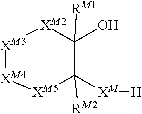

or a salt thereof, wherein each of X^{M2}, X^{M3}, X^{M4}, and X^{M5} is independently a covalent bond, optionally substituted —CH₂—or —C(R^{M})₂—, and each other variable is independently as described herein. In some embodiments, a compound has the structure of

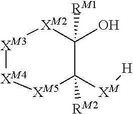

or a salt thereof, wherein each variable is independently as described herein. In some embodiments, a compound has the structure of

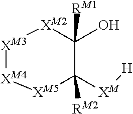

or a salt thereof, wherein each variable is independently as described herein. In some embodiments, a compound has the structure of

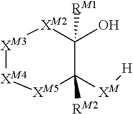

or a salt thereof, wherein each variable is independently as described herein. In some embodiments, a compound has the structure of

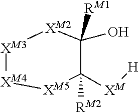

or a salt thereof, wherein each variable is independently as described herein. In some embodiments, a compound has the structure of optionally substituted

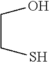

or a salt thereof. In some embodiments, a compound is

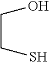

or a salt thereof. In some embodiments, a compound has the structure

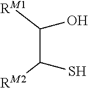

of or a salt thereof. In some embodiments, a compound has the structure of optionally substituted

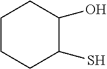

or a salt thereof. In some embodiments, a compound has the structure of optionally substituted

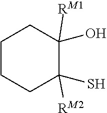

or a salt thereof. In some embodiments, a compound has the structure of optionally substituted

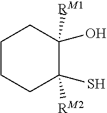

or a salt thereof. In some embodiments, a compound has the structure of optionally substituted

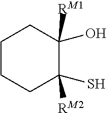

or a salt thereof. In some embodiments, a compound has the structure of optionally substituted

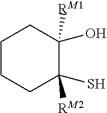

or a salt thereof. In some embodiments, a compound has the structure of optionally substituted

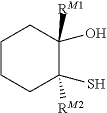

or a salt thereof. In some embodiments, a compound has the structure of

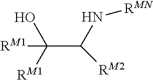

or a salt thereof, wherein each variable is independently as described herein. In some embodiments, each R^{M1} is independently R. In some embodiments, one R^{M1} is hydrogen. In some embodiments, a compound has the structure of

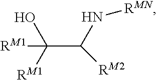

wherein each variable is independently as described herein. In some embodiments, R^{M2} and R^{MN} are taken together to form a ring as described herein. In some embodiments, a formed ring is an optionally substituted 3-30 membered ring having 0-10 heteroatoms in addition to the nitrogen. In some embodiments, a formed ring is an optionally substituted 3-10 membered saturated or partially unsaturated monocyclic ring. In some embodiments, a formed ring is an optionally substituted 3-10 membered monocyclic saturated ring. In some embodiments, a formed ring is 4-membered. In some embodiments, a formed ring is 5-membered. In some embodiments, a formed ring is 6-membered. In some embodiments, a formed ring having no heteroatoms in addition to the nitrogen. In some embodiments, a formed ring is an optionally substituted 5-membered saturated ring having no heteroatoms in addition to nitrogen. In some embodiments, R^{M1} and R^{M2} are cis. In some embodiments, In some embodiments, a compound has the structure of

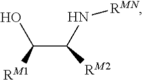

wherein R^{M2} and R^{MN} are taken together to form a ring as described herein. In some embodiments, a compound has the structure of

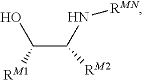

wherein R^{M2} and R^{MN} are taken together to form a ring as described herein. In some embodiments, a compound has the structure of

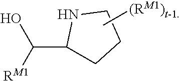

In some embodiments, a compound has the structure of

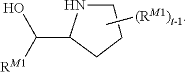

In some embodiments, a compound has the structure of

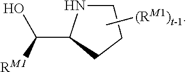

In some embodiments, a compound has the structure of

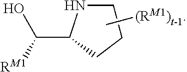

In some embodiments, a compound has the structure of optionally substituted

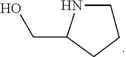

In some embodiments, a compound has the structure of optionally substituted

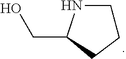

In some embodiments, a compound has the structure of optionally substituted

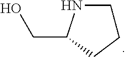

In some embodiments, R^{M1} is —CH₂—Si(R)₃, wherein the —CH₂— is optionally substituted, and each R is not hydrogen. In some embodiments, R^{M1} is —CH₂—SiPh₂Me. In some embodiments, R^{M1} comprises an electron-withdrawing group, for example, in some embodiments, R^{M1} is —CH₂—SO₂R, wherein the —CH₂— is optionally substituted. In some embodiments, R^{M1} is —CH₂—SO₂R, wherein R is as described herein and is not —H. In some embodiments, R is optionally substituted C₁₋₆ aliphatic. In some embodiments, R is optionally substituted C₁₋₆ alkyl. In some embodiments, R is optionally substituted phenyl. In some embodiments, R is phenyl.

In some embodiments, a compound has the structure of

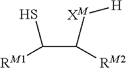

or a salt thereof, wherein each variable is independently as described herein. In some embodiments, a compound has the structure of

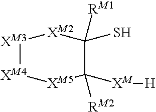

or a salt thereof, wherein each of X^{M2}, X^{M3}, X^{M4}, and X^{M5} is independently a covalent bond, optionally substituted —CH₂—or —C(R^{M})₂—, and each other variable is independently as described herein. In some embodiments, a compound has the structure of

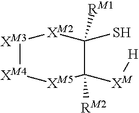

salt thereof, wherein each variable is independently as described herein. In some embodiments, a compound has the structure of

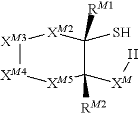

or a salt thereof, wherein each variable is independently as described herein. In some embodiments, a compound has the structure of

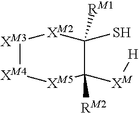

or a salt thereof, wherein each variable is independently as described herein. In some embodiments, a compound has the structure of

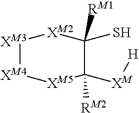

or a salt thereof, wherein each variable is independently as described herein. In some embodiments, a compound has the structure of optionally substituted

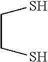

or a salt thereof. In some embodiments, a compound is

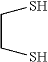

or a salt thereof. In some embodiments, a compound has the structure of

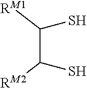

of or a salt thereof. In some embodiments, a compound has the structure of optionally substituted

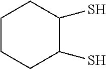

or a salt thereof. In some embodiments, a compound has the structure of optionally substituted

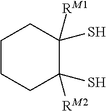

or a salt thereof. In some embodiments, a compound has the structure of optionally substituted

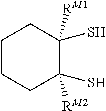

or a salt thereof. In some embodiments, a compound has the structure of optionally substituted

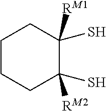

or a salt thereof. In some embodiments, a compound has the structure of optionally substituted

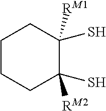

or a salt thereof. In some embodiments, a compound has the structure of optionally substituted

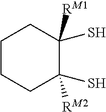

or a salt thereof. In some embodiments, a compound has the structure of

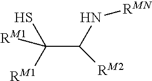

or a salt thereof, wherein each variable is independently as described herein. In some embodiments, each R^{M1} is independently R. In some embodiments, one R^{M1} is hydrogen. In some embodiments, a compound has the structure of

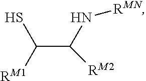

wherein each variable is independently as described herein. In some embodiments, R^{M2} and R^{MN} are taken together to form a ring as described herein. In some embodiments, a formed ring is an optionally substituted 3-30 membered ring having 0-10 heteroatoms in addition to the nitrogen. In some embodiments, a formed ring is an optionally substituted 3-10 membered saturated or partially unsaturated monocyclic ring. In some embodiments, a formed ring is an optionally substituted 3-10 membered monocyclic saturated ring. In some embodiments, a formed ring is 4-membered. In some embodiments, a formed ring is 5-membered. In some embodiments, a formed ring is 6-membered. In some embodiments, a formed ring having no heteroatoms in addition to the nitrogen. In some embodiments, a formed ring is an optionally substituted 5-membered saturated ring having no heteroatoms in addition to nitrogen. In some embodiments, R^{M1} and R^{M2} are cis. In some embodiments, In some embodiments, a compound has the structure of

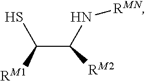

wherein R^{M2} and RM^{N} are taken together to form a ring as described herein. In some embodiments, a compound has the structure of

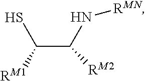

wherein R^{M2} and R^{MN} are taken together to form a ring as described herein. In some embodiments, a compound has the structure of

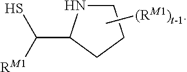

In some embodiments, a compound has the structure of

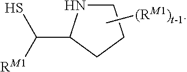

In some embodiments, a compound has the structure of

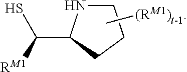

In some embodiments, a compound has the structure of

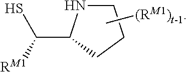

In some embodiments, a compound has the structure of optionally substituted

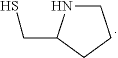

In some embodiments, a compound has the structure of optionally substituted

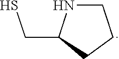

In some embodiments, a compound has the structure of optionally substituted

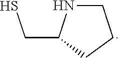

In some embodiments, R^{M1} is —CH₂—Si(R)₃, wherein the —CH₂— is optionally substituted, and each R is not hydrogen. In some embodiments, R^{M1} is —CH₂—SiPh₂Me. In some embodiments, R^{M1} comprises an electron-withdrawing group, for example, in some embodiments, R^{M1} is —CH₂—SO₂R, wherein the —CH₂— is optionally substituted. In some embodiments, R^{M1} is —CH₂—SO₂R, wherein R is as described herein and is not —H. In some embodiments, R is optionally substituted C₁₋₆ aliphatic. In some embodiments, R is optionally substituted C₁₋₆ alkyl. In some embodiments, R is optionally substituted phenyl. In some embodiments, R is phenyl.

In some embodiments, variables, e.g., R^{M1}, R^{M2}, X^{M2}, X^{M3}, X^{M4}, X^{M5}, etc., are independently as described, e.g., as in relevant sections for formula M-I or M-II. In some embodiments, —X^{M}—is —S—. In some embodiments, —X^{M}—is —CH₂—S—, wherein the —CH₂— is optionally substituted. In some embodiments, —X^{M}—is —NO^{N}—.

In some embodiments, an auxiliary compound is selected from:

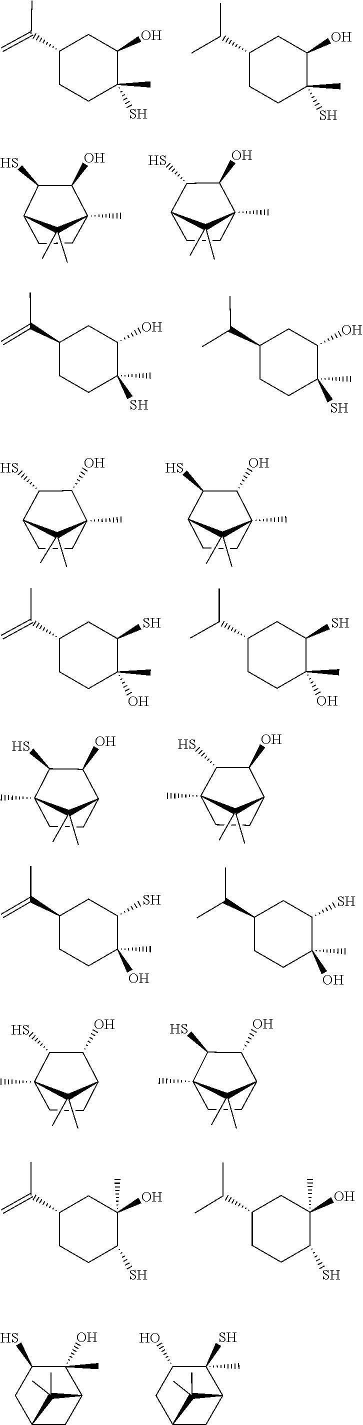

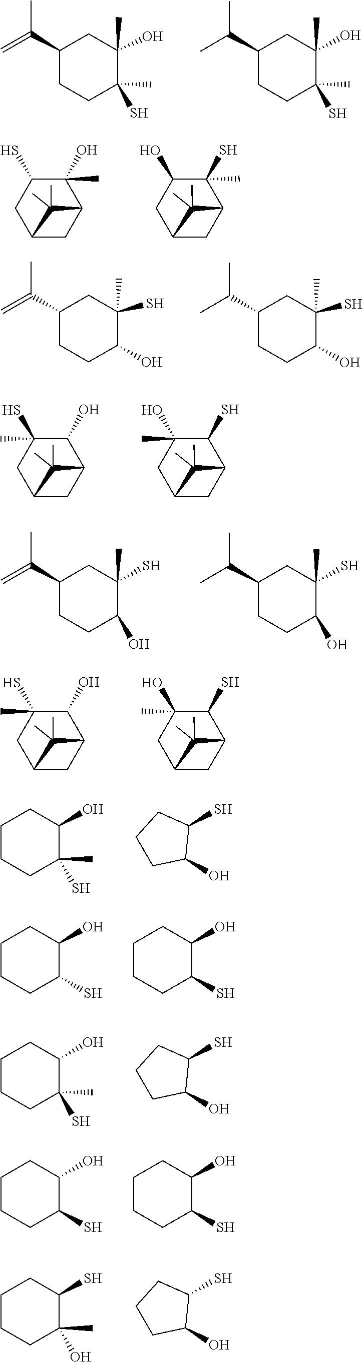

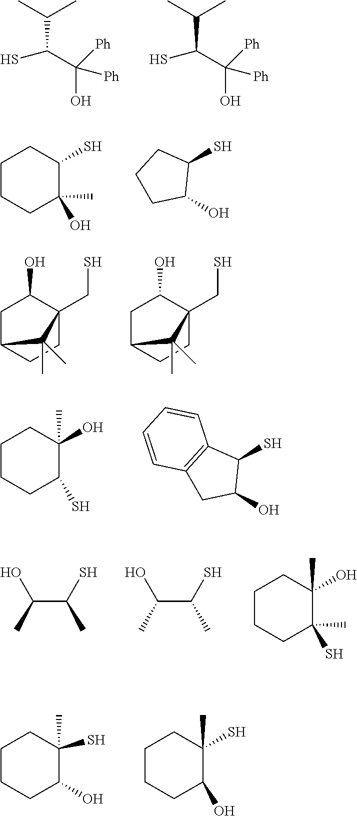

In some embodiments, an auxiliary moiety is derivatized from a compound of formula AC-I (e.g., a compound of AC-I-a, AC-I-b, AC-I-c, AC-I-d, or AC-I-e) or a salt thereof. In some embodiments, an auxiliary moiety is monovalent. In some embodiment, an auxiliary moiety has the structure of —O—X^{CA1}-L^{RM}-X^{CA2}—X^{M}—H. In some embodiment, an auxiliary moiety has the structure of H—O—X^{CA1}-L^{RM}-X^{CA2}—X^{M}—. In some embodiments, an auxiliary moiety is bivalent. In some embodiment, an auxiliary moiety has the structure of —O—X^{CA1}-L^{RM}-X^{CA2}—X^{M}—. In some embodiments, X^{M} is —S—. In some embodiments, X^{M} is —NR^{MN}—, wherein R^{MN} may form a ring with R^{M2}, X^{M2}, X^{M3}, X^{M4}, and/or X^{M5}.

In some embodiments, the present disclosure provides technologies for preparing auxiliary compounds. For example, in some embodiments, the present disclosure provides methods for preparing a compound of formula AC-I-c, AC-I-d, or AC-I-e, or a salt thereof, wherein X^{M} is —S—, comprising:

contacting a compound having the structure of:

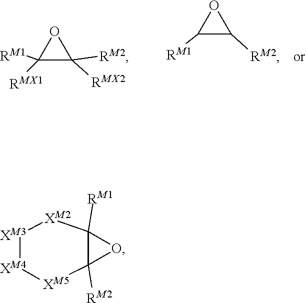

or a salt thereof, with H₂S or salt thereof.

In some embodiments, a method comprises contacting with a salt of H₂S in a solvent comprising water. In some embodiments, a salt is Na₂S. Various embodiments of the variables are described in the present disclosure, e.g., in relevant sections for coupling partner compounds or auxiliary compounds.

In some embodiments, the present disclosure provides methods for preparing a compound of formula AC-I-c, AC-I-d, or AC-I-e, or a salt thereof, wherein X^{M} is —S—, comprising:

contacting a compound having the structure of:

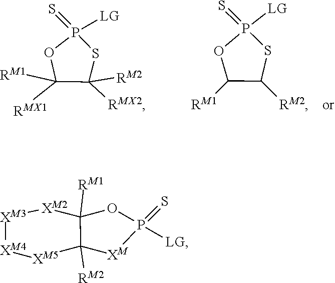

or a salt thereof, with a reducing agent.

In some embodiments, a leaving group is —S—R, wherein R is optionally substituted phenyl. In some embodiments, R is phenyl substituted with one or more electron-withdrawing groups. In some embodiments, R is phenyl substituted with five —F.

### Cycles

As appreciated by those skilled in the art, preparation of oligonucleotides typically utilizes one or more cycles. In some embodiments, the present disclosure provides cycles useful for preparation of oligonucleotides, particularly those comprising various sugar and/or internucleotidic linkages as described herein. Among other things, provided technologies can provide significantly improved yield, purity, selectivity and/or chemical compatibility compared to prior technologies.

For example, in some embodiments, a provided cycle comprises:

1) coupling;

2) capping; and

3) deprotection.

In some embodiments, a coupling step is a coupling as described herein, e.g., comprising contacting a nucleoside (e.g., of an oligonucleotide chain to be extended) with a coupling partner compound as described herein. In some embodiments, a coupling partner compound is a compound of formula M-I, M-II, or M-III, or a salt thereof, wherein P^{L} is P(═W), P->B(-L^{L}-R¹)₃, or P^{N}. In some embodiments, X^{N} is O or S and X^{M} is S. In some embodiments, X^{N} is 0 and X^{M} is S. In some embodiments, X^{N} is S and X^{M} is S. In some embodiments, X^{N} is O or S and X^{M} is N. In some embodiments, in a cycle comprising a coupling step in which a coupling partner compound comprising P(═W), P->B(-L^{L}-R^{L})₃, or P^{N} is utilized, such a cycle may comprise no modifying step which converts P^{L} which is P to P^{L} which is P(═W), P->B(-L^{L}-R¹)₃, or P^{N}. In some embodiments, a coupling partner compound is a compound of formula M-III or a salt thereof. In some embodiments, in a cycle comprising a coupling step in which a coupling partner compound comprising P(═W), P->B(-L^{L}-R¹)₃, or P^{N} is utilized, such a cycle comprises no modifying steps (e.g., those modifying internucleotidic linkages formed during coupling steps).

For example, in some embodiments, a cycle is as described in Scheme 3, 4 or 5. Those skilled in the art appreciate that various suitable coupling partner compounds can be utilized similarly to prepare various internucleotidic linkages linking various nucleosides.

In some embodiments, a provided cycle comprises:

1) coupling;

2) capping;

3) modifying; and

4) deprotection.

In some embodiments, in such a cycle, a coupling step comprises contacting a nucleoside (e.g., of an oligonucleotide chain to be extended) with a coupling partner compound, e.g., a compound of formula M-I, or M-II, or a salt thereof, wherein P^{L} is P. In some embodiments, X^{N} is O or S and X^{M} is S. In some embodiments, X^{N} is O and X^{M} is S. In some embodiments, X^{N} is S and X^{M} is S. In some embodiments, X^{N} is O or S and X^{M} is N. In some embodiments, in a cycle comprising a coupling step in which a coupling partner compound comprising P^{L} which is P is utilized, such a cycle may comprise a modifying step which modifies an internucleotidic linkage formed during a coupling step, e.g., a modifying step which converts P^{L} which is P to P^{L} which is P(═W), P->B(-L^{L}-R¹)₃, or P^{N}. In some embodiments, a modifying step is sulfurization (converting P to P═S). In some embodiments, a modifying step is oxidation (converting P to P═O). In some embodiments, a modifying step comprises contact with a compound comprising —N3 (e.g., ADIH, which may convert P to P^{N} (e.g., P^{N} comprising P═N—). In some embodiments, in such cycles, there can be a second capping step after a modifying step. In some embodiments, a capping step before a modifying step comprises an amidation condition. In some embodiments, an amidation condition preferentially caps amino groups over hydroxyl groups. In some embodiments, a second capping step comprises an esterification condition. In some embodiments, an esterification condition caps —OH, e.g., unreacted 5′-OH groups. Various cycles, including useful steps, reagents, conditions, etc. are described in U.S. Pat. Nos. 9,394,333, 9,744,183, 9,605,019, 9,598,458, 9,982,257, 10,160,969, 104,799,95, US 2020/0056173, US 2018/0216107, US 2019/0127733, U.S. Pat. No. 10,450,568, US 2019/0077817, US 2019/0249173, US 2019/0375774,a WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the cycles of each of which are incorporated herein by reference.

In some embodiments, a deprotection group de-protects a protected hydroxyl group, e.g., 5′-DMTrO—, such that the deprotected —OH can be utilized, e.g., for further cycles, cleavage and deprotection, etc., as desired.

In some embodiments, provided technologies comprise one or more cycles comprising modifying steps and one or more cycles comprising no modifying steps. In some embodiments, one or more steps are independently chirally controlled.

Various technologies can be utilized for production of oligonucleotides and compositions in accordance with the present disclosure. For example, traditional phosphoramidite chemistry can be utilized to prepare stereorandom oligonucleotides and compositions, and certain reagents and chirally controlled technologies can be useful for preparing chirally controlled oligonucleotide compositions (e.g., constructing internucleotidic linkages linking ribose sugars). Certain useful technologies are described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774,a WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the reagents and methods of each of which are incorporated herein by reference.

In some embodiments, chirally controlled/stereoselective preparation of oligonucleotides and compositions thereof comprise utilization of a chiral auxiliary, e.g., as part of coupling partner compound, e.g., monomeric phosphoramidites. In some embodiments, a chiral auxiliary is a compound of formula AC-1, AC-I-a, AC-I-b, AC-I-c, AC-I-d, or AC-I-3e, or a salt thereof, wherein the compound is chiral. Examples of additional chiral auxiliary reagents and coupling partner compounds, e.g., phosphoramidites, that can be useful in accordance with the present disclosure include those described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the chiral auxiliary reagents and phosphoramidites of each of which are independently incorporated herein by reference. In some embodiments, a chiral auxiliary is

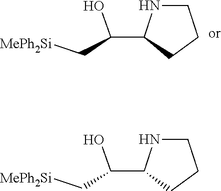

(DPSE chiral auxiliaries). In some embodiments, a chiral auxiliary is

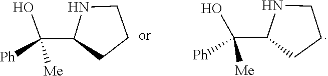 [SMILES: C[C@@](O)(c1ccccc1)[C@@H]1CCCN1 ; C[C@](O)(c1ccccc1)[C@H]1CCCN1]

In some embodiments, a chiral auxiliary is

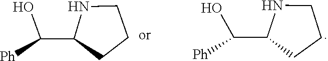 [SMILES: O[C@H](c1ccccc1)[C@@H]1CCCN1 ; O[C@@H](c1ccccc1)[C@H]1CCCN1]

In some embodiments, a chiral auxiliary comprises —SO₂R^{AU}, wherein R^{AU} is an optionally substituted group selected from C₁₋₂₀ aliphatic, C₁₋₂₀ heteroaliphatic having 1-10 heteroatoms, C₆₋₂₀ aryl, C₆₋₂₀ arylaliphatic, C₆₋₂₀ arylheteroaliphatic having 1-10 heteroatoms, 5-20 membered heteroaryl having 1-10 heteroatoms, and 3-20 membered heterocyclyl having 1-10 heteroatoms. In some embodiments, a chiral auxiliary is

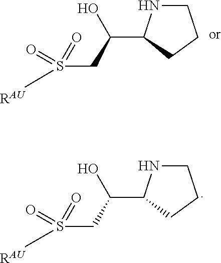

In some embodiments, R^{AU} is optionally substituted aryl. In some embodiments, R^{AU} is optionally substituted phenyl. In some embodiments, R^{AU} is optionally substituted C₁₋₆ aliphatic. In some embodiments, a chiral auxiliary is

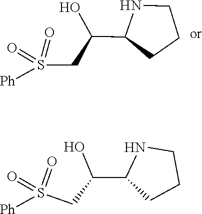 [SMILES: O=S(=O)(C[C@@H](O)[C@@H]1CCCN1)c1ccccc1 ; O=S(=O)(C[C@H](O)[C@H]1CCCN1)c1ccccc1]

(PSM chiral auxiliaries). In some embodiments, utilization of such chiral auxiliaries, e.g., preparation, phosphoramidites comprising such chiral auxiliaries, intermediate oligonucleotides comprising such auxiliaries, protection, removal, etc., is described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858 and incorporated herein by reference. In some embodiments, chiral auxiliary compounds and chiral coupling partner compounds, e.g., phosphoramidites, are provided as chirally pure compounds, e.g., with a stereopurity of about or at least about 90%, 91%, 92%, 93%, 94%, 95%, 96%, 97%, 98%, or 99%.

In some embodiments, certain useful chirally controlled preparation technologies, including oligonucleotide synthesis cycles, reagents and conditions are described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the oligonucleotide synthesis methods, cycles, reagents and conditions of each of which are independently incorporated herein by reference.

Once synthesized, oligonucleotides and compositions are typically further purified. Suitable purification technologies are widely known and practiced by those skilled in the art, including but not limited to those described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the purification technologies of each of which are independently incorporated herein by reference.

In some embodiments, a cycle comprises or consists of coupling, capping, and deblocking. In some embodiments, a cycle comprises or consists of coupling, capping, modification and deblocking. In some embodiments, a cycle comprises or consists of coupling, capping, modification, capping and deblocking. These steps are typically performed in the order they are listed, but in some embodiments, as appreciated by those skilled in the art, the order of certain steps, e.g., capping and modification, may be altered. If desired, one or more steps may be repeated to improve conversion, yield and/or purity as those skilled in the art often perform in syntheses. For example, in some embodiments, coupling may be repeated; in some embodiments, modification (e.g., oxidation to install ═O, sulfurization to install ═S, etc.) may be repeated; in some embodiments, coupling is repeated after modification which can convert a P(III) linkage to a P(V) linkage which can be more stable under certain circumstances, and coupling is routinely followed by modification to convert newly formed P(III) linkages to P(V) linkages. In some embodiments, when steps are repeated, different conditions may be employed (e.g., concentration, temperature, reagent, time, etc.).

In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, the solid support is Highly Crosslinked Polystyrene (HCP). In some embodiments, the solid support is hybrid support of Controlled Pore Glass (CPG) and Highly Cross-linked Polystyrene (HCP). In some embodiments, a solid support is a metal foam. In some embodiments, a solid support is a resin. In some embodiments, oligonucleotides are cleaved from a solid support.

In some embodiments, the present disclosure provides solid support that are particularly useful for preparing oligonucleotides and compositions of the present disclosure. For example, in some embodiments, linker used to connect nucleosides/oligonucleotides to solid support comprise —NR—, wherein R is not —H, instead of typically utilized —NH—, provide improved stability under one or more synthetic conditions, thus significantly improve yields and/or purity and dramatically reduce undesired cleavage from the solid support. In some embodiments, a provided linker is or comprises —N(R)—C(O)-L-C(O)—, wherein R is not —H. In some embodiments, L is optionally substituted bivalent C₁₋₁₀ aliphatic. In some embodiments, L is optionally substituted —(CH₂)n— wherein n is 1-20. In some embodiments, L is —(CH₂)n— wherein n is 1-20. In some embodiments, n is 2. In some embodiments, n is 3. In some embodiments, a linker is or comprises -L-N(R)—C(O)-L-C(O)—, wherein R is not —H. In some embodiments, a linker is or comprises —(CH₂)m—N(R)—C(O)-L-C(O)—, wherein each —CH₂— is independently and optionally substituted, and m is 1-30. In some embodiments, m is 1-20. In some embodiments, m is 1-10. In some embodiments, m is 1-5. In some embodiments, m is 3. In some embodiments, a linker is or comprises —(CH₂)₃—N(R)—C(O)—(CH₂)₂—C(O)— wherein R is not —H. In some embodiments, a linker is or comprises —(CH₂)₃—N(R)—C(O)—(CH₂)₆—N(R)—C(O)—(CH₂)₂—C(O)— wherein R is not —H. In some embodiments, R is optionally substituted C₁₋₁₀ aliphatic. In some embodiments, R is optionally substituted C₁₋₁₀ alkyl. In some embodiments, R is methyl. In some embodiments, R is ethyl. In some embodiments, R is isopropyl. In some embodiments, —C(O)— is connected to a nucleoside, e.g., to a 3′-carbon via oxygen.

In some embodiments, a support (e.g., a solid support, a support soluble in one or more conditions/steps but can be precipitated in other conditions/steps (e.g., various support comprising hydrophobic moieties, AJIPHASE), etc.) is utilized for preparation. In some embodiments, the present disclosure provides various support useful for oligonucleotide synthesis. In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, a solid support is Highly Crosslinked Polystyrene (HCP). In some embodiments, a solid support is hybrid support of Controlled Pore Glass (CPG) and Highly Cross-linked Polystyrene (HCP). In some embodiments, a solid support is PS5G. In In some embodiments, a solid support is PS200. In some embodiments, a solid support is CPS. In some embodiments, a solid support is a metal foam. In some embodiments, a solid support is a resin. In some embodiments, oligonucleotides are cleaved from a solid support. In some embodiments, a support is loaded with a first nucleoside (e.g., with —OH such as 5′-OH protected as —ODMTr) for synthesis. In some embodiments, a —OH, such as 5′-OH, is deprotected and ready for coupling. In some embodiments, a support is functionalized for loading of nucleosides or oligonucleotides (e.g., various universal supports such as Glen UnySupport).

In some embodiments, the present disclosure provides supports, e.g., various solid supports, that are particularly useful for preparing oligonucleotides and compositions of the present disclosure. For example, in some embodiments, linkers are used to connect nucleosides/oligonucleotides to various supports e.g., solid supports. In some embodiments, such supports are stable under various conditions, e.g., when exposed to oligonucleotide synthesis conditions comprising DBU as described herein.

In some embodiments, a linker is L as described herein. In some embodiments, a linker is or comprises a divalent moiety L^{SP}, wherein -L^{SP}- is L as described herein. In some embodiments, L^{SP} is a covalent bond or an optionally substituted, linear or branched C₁-C₃₀ alkylene, wherein one or more methylene units of -Linker— are optionally and independently replaced by an optionally substituted C₁-C6 alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—.

In some embodiments, the present disclosure provides an agent having the structure of:

S^{SP}-L^{SP}-N^{SP}

or a salt thereof, wherein:

S^{SP} is a support;

L^{SP} is a linker; and

N^{SP} is —H, hydroxyl protection group, R, an optionally substituted or protected nucleoside or nucleotide, or an oligonucleotide.

In some embodiments, S^{SP} is a suitable support for oligonucleotide synthesis as described herein, e.g., CPG, HCP, etc. described herein. In some embodiments, S^{SP} is CPG. In some embodiments, S^{SP} is PS5G. In some embodiments, S^{SP} is PS200. In some embodiments, S^{SP} is HCP. In some embodiments, S^{SP} is CPS. In some embodiments, S^{SP} is NPHL.

In some embodiments, L^{SP} is L as described herein. In some embodiments, one or more methylene units are optionally and independently replaced with —O—, —N(R′)—, —C(O)—, —N(R′)C(O)—, —N(R′)C(O)O—, or —C(O)O—.

In some embodiments, L^{SP} is or comprises —(CH₂)ₙ₁—N(R^{SP1})—C(O)—(CH₂)ₙ₂—C(O)—O—, wherein each of n1 of n2 is independently 0-20, and R^{SP1} is R′ as described herein. In some embodiments, R^{SP1} is not hydrogen. In some embodiments, L^{SP} is or comprises —(CH₂)₃—N(CH₃)—C(O)—(CH₂)₂—C(O)—O—. In some embodiments, L^{SP} is or comprises —N(R^{SP1})-(dT)ₙ₁—O—(CH₂)ₙ₂—N(R^{SP2})—C(O)—(CH₂)ₙ₃—C(O)—O—, wherein each of n1, n2 and n3 is independently 0-20, and each of R^{SP1} and R^{SP2} is independently R′ as described herein. In some embodiments, L^{SP} is or comprises -LCAA—N(R^{SP1})-(dT)ₙ₁—O—(CH₂)ₙ₂—N(R^{SP2})—C(O)—(CH₂)ₙ₃—C(O)—O—, wherein each of n1, n2 and n3 is independently 0-20, and each of R^{SP1} and R^{SP2} is independently R′ as described herein. In some embodiments, L^{SP} is or comprises -LCAA—NH—(dT)ₙ₁—O—(CH₂)ₙ₂—NH—C(O)—(CH₂)₁₁₃—C(O)—O—, wherein each of n1, n2 and n3 is independently 0-20. In some embodiments, L^{SP} is or comprises -LCAA—NH—(dT)₅—O—(CH₂)₆—NH—C(O)—(CH₂)₂—C(O)—O—. In some embodiments, L^{SP} is or comprises —N(R^{SP2})—C(O)—(CH₂)₁₁₃—C(O)—O—, wherein each variable is independently as described herein. In some embodiments, L^{SP} is or comprises —NH—C(O)—(CH₂)₂—C(O)—O—.

In some embodiments, each of n1, n2, and n3 is independently n as described herein. In some embodiments, n1 is 1, 2, 3, 4, 5, 6, 7, 8, 9, 10, 11, 12, 13, 14, 15, 16, 17, 18, 19, or 20. In some embodiments, n1 is 1-10. In some embodiments, n1 is 3-5. In some embodiments, n1 is 1. In some embodiments, n1 is 2. In some embodiments, n1 is 3. In some embodiments, n1 is 4. In some embodiments, n1 is 5. In some embodiments, n2 is 1, 2, 3, 4, 5, 6, 7, 8, 9, 10, 11, 12, 13, 14, 15, 16, 17, 18, 19, or 20. In some embodiments, n2 is 5-20. In some embodiments, n2 is 10-20. In some embodiments, n2 is 10. In some embodiments, n2 is 11. In some embodiments, n2 is 12. In some embodiments, n2 is 13. In some embodiments, n2 is 14. In some embodiments, n2 is 15. In some embodiments, n2 is 16. In some embodiments, n3 is 1, 2, 3, 4, 5, 6, 7, 8, 9, 10, 11, 12, 13, 14, 15, 16, 17, 18, 19, or 20. In some embodiments, n3 is 1-10. In some embodiments, n3 is 3-5. In some embodiments, n3 is 1. In some embodiments, n3 is 2. In some embodiments, n3 is 3. In some embodiments, n3 is 4. In some embodiments, n3 is 5. In some embodiments, n1 is 3. In some embodiments, n2 is 2. In some embodiments, n1 is 5, n2 is 6 and n3 is 2.

In some embodiments, R^{SP1} is optionally substituted C₁₋₆ aliphatic. In some embodiments, R^{SP1} is optionally substituted C₁₋₆ alkyl. In some embodiments, R^{SP1} is methyl. In some embodiments, R^{SP1} is —H. In some embodiments, R^{SP2} is optionally substituted C₁₋₆ aliphatic. In some embodiments, R^{SP2} is optionally substituted C₁₋₆ alkyl. In some embodiments, R^{SP2} is methyl. In some embodiments, R^{SP2} is —H.

In some embodiments, L^{SP} is or comprises —(CH₂)ₙ₁—N(R^{SP1})—C(O)—O—(CH₂)ₙ₂-N(R^{SP2})—C(O)—(CH₂)₁₁₃—C(O)—O—, wherein each variable is independently as described herein. In some embodiments, each of R^{SP1} and R^{SP2} is hydrogen. In some embodiments, n1 is 3. In some embodiments, n2 is 6. In some embodiments, n2 is 10. In some embodiments, n2 is 14. In some embodiments, n3 is 2. In some embodiments, L^{SP} is or comprises —(CH₂)₃—NHC(O)O—(CH₂)₆—NHC(O)(CH₂)₂—C(O)O—. In some embodiments, L^{SP} is or comprises —(CH₂)₃—NHC(O)O—(CH₂)₁₀—NHC(O)(CH₂)₂—C(O)O—. In some embodiments, L^{SP} is or comprises —(CH₂)₃—NHC(O)O—(CH₂)₁₄—NHC(O)(CH₂)₂—C(O)O—.

In some embodiments, L^{SP} is or comprises —(CH₂)ₙ₁—N(R^{SP1})—C(O)—(OCH₂CH₂)ₙ₂—N(R^{SP2})—C(O)—(CH₂)ₙ₃—C(O)—O—, wherein each variable is independently as described herein. In some embodiments, each of R^{SP1} and R^{SP2} is hydrogen. In some embodiments, n1 is 3. In some embodiments, n2 is 3. In some embodiments, n2 is 4. In some embodiments, n3 is 2. In some embodiments, L^{SP} is or comprises —(CH₂)₃—NHC(O)—(OCH₂CH₂)₃—NH—C(O)—(CH₂)₂—C(O)O—. In some embodiments, L^{SP} is or comprises —(CH₂)₃—NHC(O)—(OCH₂CH₂)₄—NH—C(O)—(CH₂)₂—C(O)O—.

In some embodiments, L^{SP} is or comprises —(CH₂)ₙ₁—N(R^{SP1})—C(O)—N(R^{SP2})—(CH₂)ₙ₁—CH(OR′)—(CH₂)₁₁₃—O—, wherein each variable is independently as described herein. In some embodiments, each of R^{SP1} and R^{SP2} is hydrogen. In some embodiments, n1 is 1. In some embodiments, n1 is 3. In some embodiments, n2 is 1. In some embodiments, n2 is 3. In some embodiments, n2 is 4. In some embodiments, n3 is 1. In some embodiments, n3 is 2. In some embodiments, R′ is —C(O)R. In some embodiments, R is optionally substituted C₁₋₆ aliphatic. In some embodiments, R is optionally substituted C₁₋₆ alkyl. In some embodiments, R is optionally substituted methyl. In some embodiments, R is —CHCl₂. In some embodiments, L^{SP} is or comprises —CH₂—NH—C(O)—NH—CH₂—CH[OC(O)CCl₂]—CH₂—O—. In some embodiments, L^{SP} is or comprises —(CH₂)₃—NH—C(O)—NH—CH₂—CH[OC(O)CCl₂]—CH₂—O—.

In some embodiments, L^{SP} is or comprises -Cy-as described herein.

In some embodiments, -Cy-is optionally substituted 5-10 membered heterocyclylene having 1-2 heteroatoms independently selected from oxygen, nitrogen, sulfur. In some embodiments, -Cy- is optionally substituted 5-membered heterocyclylene having 1-2 heteroatoms independently selected from oxygen, nitrogen, sulfur. In some embodiments, -Cy- is optionally substituted 6-membered heterocyclylene having 1-2 heteroatoms independently selected from oxygen, nitrogen, sulfur. In some embodiments, -Cy- is optionally substituted

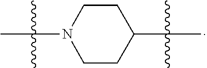 [SMILES: *C1CCN(*)CC1]

In some embodiments, -Cy- is

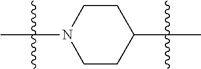 [SMILES: *C1CCN(*)CC1]

In some embodiments, -Cy- is optionally substituted

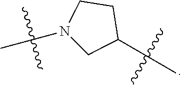 [SMILES: *C1CCN(*)C1]

In some embodiments, -Cy- is

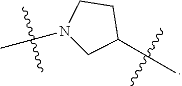 [SMILES: *C1CCN(*)C1]

In some embodiments, -Cy- is optionally substituted

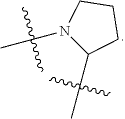 [SMILES: *C1CCCN1*]

In some embodiments, -Cy- is

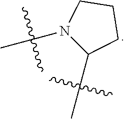 [SMILES: *C1CCCN1*]

In some embodiments, -Cy- is optionally substituted

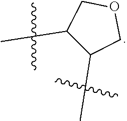 [SMILES: *C1COCC1*]

In some embodiments, -Cy- is

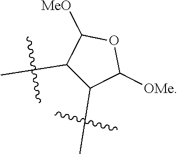 [SMILES: *C1C(OC)OC(OC)C1*]

In some embodiments, -Cy- is

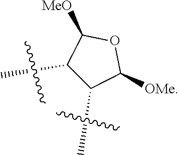

In some embodiments, -Cy- is optionally substituted 5-30 membered heteroarylene having 1-10 heteroatoms independently selected from oxygen, nitrogen and sulfur. In some embodiments, -Cy- is optionally substituted 5-10 membered heteroarylene having 1-2 heteroatoms independently selected from oxygen, nitrogen, sulfur. In some embodiments, -Cy- is optionally substituted

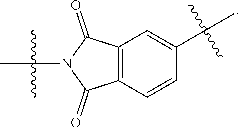 [SMILES: *c1ccc2c(c1)C(=O)N(*)C2=O]

In some embodiments, -Cy- is

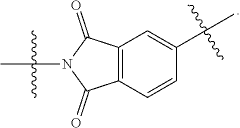 [SMILES: *c1ccc2c(c1)C(=O)N(*)C2=O]

In some embodiments, -Cy- is an optionally substituted 4-10 membered saturated monocyclic having 0-4 heteroatoms. In some embodiments, -Cy- is an optionally substituted 4-7 membered saturated monocyclic having a nitrogen atom, wherein -Cy- is connected at the nitrogen atom. In some embodiments, -Cy- is an optionally substituted bivalent ring selected from 3-30 membered carbocyclylene, 6-30 membered arylene, 5-30 membered heteroarylene having 1-10 heteroatoms independently selected from oxygen, nitrogen and sulfur, and 3-30 membered heterocyclylene having 1-10 heteroatoms, e.g., independently selected from oxygen, nitrogen, sulfur, phosphorus and silicon. In some embodiments, -Cy- is optionally substituted

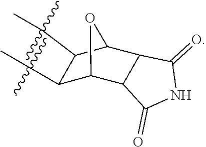 [SMILES: *C1C(*)C2OC1C1C(=O)NC(=O)C21]

In some embodiments, -Cy- is

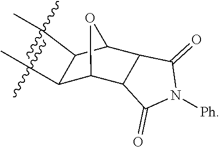 [SMILES: *C1C(*)C2OC1C1C(=O)N(c3ccccc3)C(=O)C21]

In some embodiments, -Cy- is

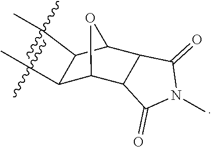 [SMILES: *C1C(*)C2OC1C1C(=O)N(C)C(=O)C21]

In some embodiments, -Cy- is

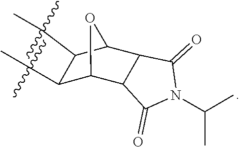 [SMILES: *C1C(*)C2OC1C1C(=O)N(C(C)C)C(=O)C21]

In some embodiments, L^{SP} is or comprises —N(R^{SP1})—C(O)-Cy-C(O)—(CH₂)ₙ₃—C(O)O—. In some embodiments, R^{SP1} is —H. In some embodiments, n3 is 2. In some embodiments, L^{SP} is or comprises

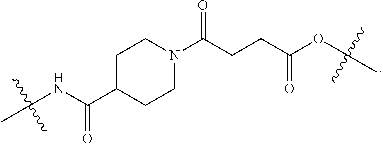 [SMILES: *NC(=O)C1CCN(C(=O)CCC(=O)O*)CC1]

In some embodiments, L^{SP} is optionally substituted

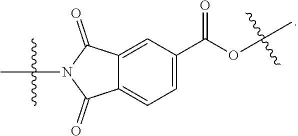 [SMILES: *OC(=O)c1ccc2c(c1)C(=O)N(*)C2=O]

In some embodiments, L^{SP} is

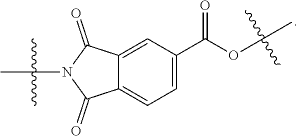 [SMILES: *OC(=O)c1ccc2c(c1)C(=O)N(*)C2=O]

In some embodiments, L^{SP} is or comprises -O-Cy-O—. In some embodiments, L^{SP} is or comprises —C(O)O-Cy-O—. In some embodiments, L^{SP} is or comprises —N(R^{SP1})—C(O)—CH₂)ₙ₃—C(O)O-Cy-O—, wherein each variable is as described herein. In some embodiments, L^{SP} is or comprises —(CH₂)ₙ₁—N(R^{SP1})—C(O)—(CH₂)ₙ₃—C(O)O-Cy-O—, wherein each variable is as described herein. In some embodiments, L^{SP} is or comprises —(CH₂)ₙ₁—NH—C(O)—(CH₂)ₙ₃—C(O)O-Cy-O—, wherein each variable is as described herein. In some embodiments, L^{SP} is or comprises —(CH₂)ₙ₁—N(R^{SP1})—C(O)—(CH₂)ₙ₂—N(R^{SP2})—C(O)—(CH₂)ₙ₃—C(O)O-Cy-O—, wherein each variable is as described herein. In some embodiments, L^{SP} is or comprises —(CH₂)ₙ₁—NH—C(O)O—(CH₂)ₙ₂—NH—C(O)—CH₂)ₙ₃—C(O)O-Cy-O—, wherein each variable is as described herein. In some embodiments, L^{SP} is or comprises —(CH₂)ₙ₁—N(R^{SP1})—(CH₂)ₙ₂—N(R^{SP2})—C(O)—CH₂)ₙ₃—C(O)O-Cy-O—, wherein each variable is as described herein. In some embodiments, L^{SP} is or comprises —(CH₂)ₙ₁—NH—(CH₂)ₙ₂—NH—C(O)—CH₂)ₙ₃—C(O)O-Cy-O—, wherein each variable is as described herein. In some embodiments, each of n1, n2, and n3 is independently n as described herein. In some embodiments, n1 is 1, 2, 3, 4, 5, 6, 7, 8, 9, 10, 11, 12, 13, 14, 15, 16, 17, 18, 19, or 20. In some embodiments, n1 is 1-10. In some embodiments, n1 is 3-5. In some embodiments, n1 is 1. In some embodiments, n1 is 2. In some embodiments, n1 is 3. In some embodiments, n1 is 4. In some embodiments, n1 is 5. In some embodiments, n2 is 1, 2, 3, 4, 5, 6, 7, 8, 9, 10, 11, 12, 13, 14, 15, 16, 17, 18, 19, or 20. In some embodiments, n2 is 5-20. In some embodiments, n2 is 10-20. In some embodiments, n2 is 10. In some embodiments, n2 is 11. In some embodiments, n2 is 12. In some embodiments, n2 is 13. In some embodiments, n2 is 14. In some embodiments, n2 is 15. In some embodiments, n2 is 16. In some embodiments, n3 is 1, 2, 3, 4, 5, 6, 7, 8, 9, 10, 11, 12, 13, 14, 15, 16, 17, 18, 19, or 20. In some embodiments, n3 is 1-10. In some embodiments, n3 is 3-5. In some embodiments, n3 is 1. In some embodiments, n3 is 2. In some embodiments, n3 is 3. In some embodiments, n3 is 4. In some embodiments, n3 is 5. In some embodiments, n1 is 3. In some embodiments, n2 is 2. In some embodiments, n3 is 2. In some embodiments, R^{SP1} is optionally substituted C₁₋₆ aliphatic. In some embodiments, R^{P}′ is optionally substituted C₁₋₆ alkyl. In some embodiments, R^{SP1} is methyl. In some embodiments, R^{SP2} is optionally substituted C₁₋₆ aliphatic. In some embodiments, R^{SP2} is optionally substituted C₁₋₆ alkyl. In some embodiments, R^{SP2} is methyl. In some embodiments, -Cy- is optionally substituted

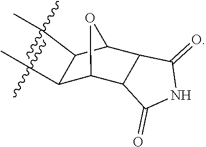 [SMILES: *C1C(*)C2OC1C1C(=O)NC(=O)C21]

In some embodiments, -Cy- is

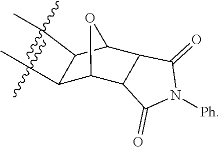 [SMILES: *C1C(*)C2OC1C1C(=O)N(c3ccccc3)C(=O)C21]

In some embodiments, -Cy- is

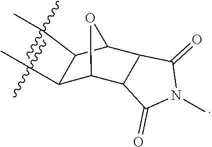 [SMILES: *C1C(*)C2OC1C1C(=O)N(C)C(=O)C21]

In some embodiments, -Cy- is

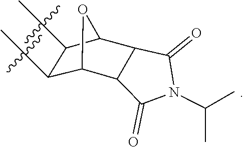 [SMILES: *C1C(*)C2OC1C1C(=O)N(C(C)C)C(=O)C21]

In some embodiments, L^{SP} comprises

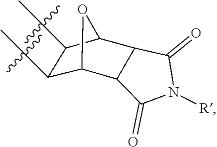

wherein R′ is as described herein. In some embodiments, R′ is optionally substituted C₁₋₆ aliphatic. In some embodiments, R′ is optionally substituted C₁₋₆ alkyl. In some embodiments, R′ is isopropyl. In some embodiments, R′ is methyl. In some embodiments, R′ is optionally substituted phenyl. In some embodiments, R′ is phenyl. In some embodiments, L^{SP} is or comprises

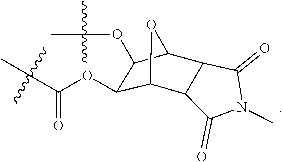 [SMILES: *OC1C(OC(*)=O)C2OC1C1C(=O)N(C)C(=O)C21]

In some embodiments, L^{SP} is or comprises

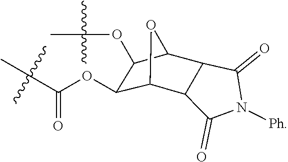 [SMILES: *OC1C(OC(*)=O)C2OC1C1C(=O)N(c3ccccc3)C(=O)C21]

In some embodiments, L^{SP} is or comprises

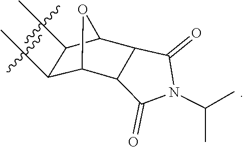 [SMILES: *C1C(*)C2OC1C1C(=O)N(C(C)C)C(=O)C21]

In some embodiments, L^{SP} is or comprises

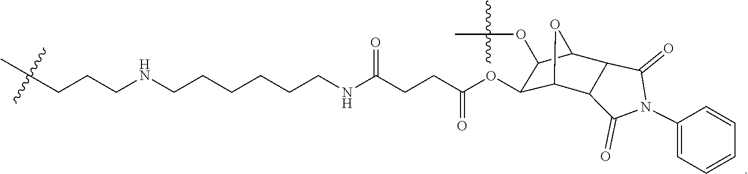 [SMILES: *CCCNCCCCCCNC(=O)CCC(=O)OC1C(O*)C2OC1C1C(=O)N(c3ccccc3)C(=O)C21]

In some embodiments, L^{SP} is or comprises

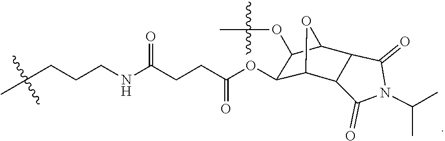 [SMILES: *CCCNC(=O)CCC(=O)OC1C(O*)C2OC1C1C(=O)N(C(C)C)C(=O)C21]

In some embodiments, -Cy- is optionally substituted

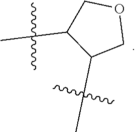 [SMILES: *C1COCC1*]

In some embodiments, -Cy- is

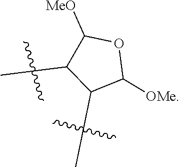 [SMILES: *C1C(OC)OC(OC)C1*]

In some embodiments, -Cy- is

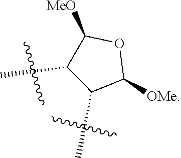

In some embodiments, L^{SP} is or comprises

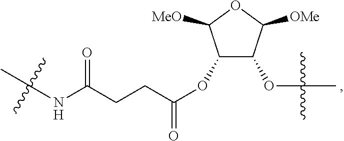 [SMILES: *NC(=O)CCC(=O)O[C@@H]1[C@@H](OC)O[C@@H](OC)[C@@H]1O*]

wherein —O— is bonded to N^{SP}. In some embodiments, L^{SP} is or comprises

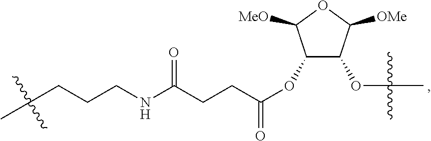 [SMILES: *CCCNC(=O)CCC(=O)O[C@@H]1[C@@H](OC)O[C@@H](OC)[C@@H]1O*]

wherein —O— is bonded to N^{SP}.

In some embodiments, L^{SP} is or comprises

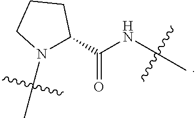 [SMILES: *NC(=O)[C@H]1CCCN1*]

In some embodiments, L^{SP} is or comprises

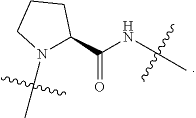 [SMILES: *NC(=O)[C@@H]1CCCN1*]

In some embodiments, L^{SP} is or comprises

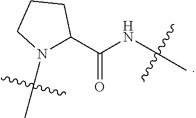 [SMILES: *NC(=O)C1CCCN1*]

In some embodiments, L^{SP} is or comprises

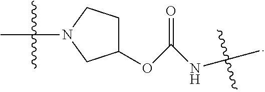 [SMILES: *NC(=O)OC1CCN(*)C1]

In some embodiments, L^{SP} is or comprises

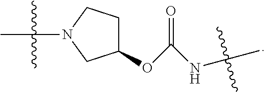 [SMILES: *NC(=O)O[C@@H]1CCN(*)C1]

In some embodiments, L^{SP} is or comprises

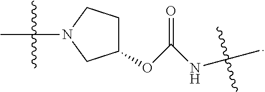 [SMILES: *NC(=O)O[C@H]1CCN(*)C1]

In some embodiments, L^{SP} is or comprises

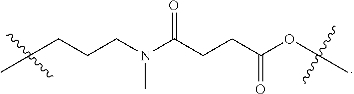 [SMILES: *CCCN(C)C(=O)CCC(=O)O*]

In some embodiments, L^{SP} is or

comprises

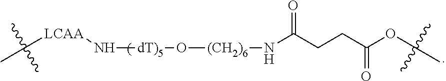

In some embodiments, L^{SP} is or comprises

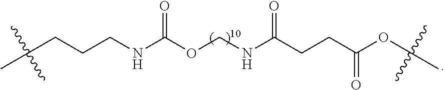

In some embodiments, L^{SP} is or comprises

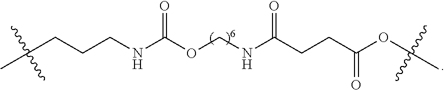

In some embodiments, L^{SP} is or comprises

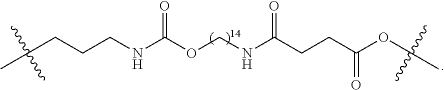

In some embodiments, L^{SP} is or comprises

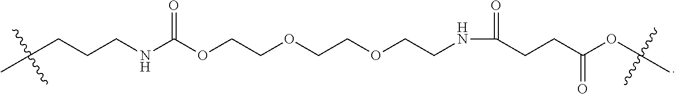 [SMILES: *CCCNC(=O)OCCOCCOCCNC(=O)CCC(=O)O*]

In some embodiments, L^{SP} is or comprises

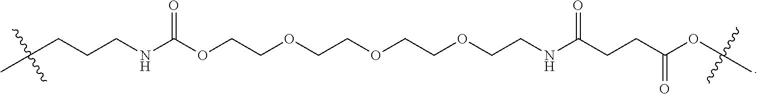 [SMILES: *CCCNC(=O)OCCOCCOCCOCCNC(=O)CCC(=O)O*]

In some embodiments, L^{SP} is or comprises

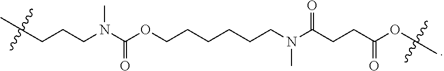 [SMILES: *CCCN(C)C(=O)OCCCCCCN(C)C(=O)CCC(=O)O*]

In some embodiments, L^{SP} is or comprises

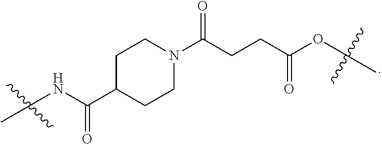 [SMILES: *NC(=O)C1CCN(C(=O)CCC(=O)O*)CC1]

In some embodiments, L^{SP} is or comprises

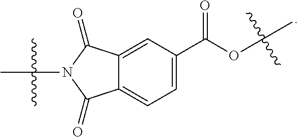 [SMILES: *OC(=O)c1ccc2c(c1)C(=O)N(*)C2=O]

In some embodiments, L^{SP} is or comprises

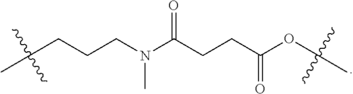 [SMILES: *CCCN(C)C(=O)CCC(=O)O*]

In some embodiments, L^{SP} is or comprises

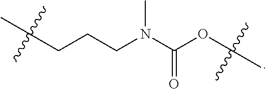 [SMILES: *CCCN(C)C(=O)O*]

In some embodiments, L^{SP} is or comprises

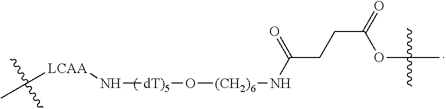

In some embodiments, L^{SP} is or comprises

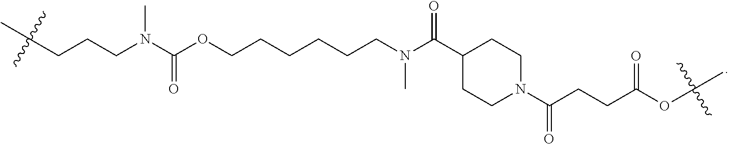 [SMILES: *CCCN(C)C(=O)OCCCCCCN(C)C(=O)C1CCN(C(=O)CCC(=O)O*)CC1]

In some embodiments, L^{SP} is or comprises

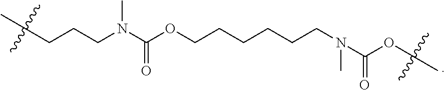 [SMILES: *CCCN(C)C(=O)OCCCCCCN(C)C(=O)O*]

In some embodiments, L^{SP} is or comprises

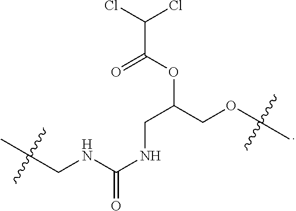 [SMILES: *CNC(=O)NCC(CO*)OC(=O)C(Cl)Cl]

In some embodiments, L^{SP} is or comprises

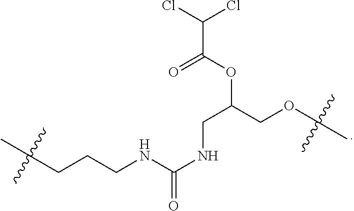 [SMILES: *CCCNC(=O)NCC(CO*)OC(=O)C(Cl)Cl]

In some embodiments, L^{SP} is bonded to N^{NS} through an oxygen atom of L^{SP}. In some embodiments, —O— is connected to N^{SP}, e.g., —H, -DMTr, an optionally substituted or protected nucleoside or nucleotide, or an oligonucleotide. In some embodiments, N^{Ns} is —H. In some embodiments, N^{Ns} is —H, and the hydrogen is bonded to an oxygen atom of L^{SP} to form a —OH for coupling with a coupling partner, e.g., a phosphoramidite. In some embodiments, N^{NS} is an optionally substituted or protected nucleotide. In some embodiments, N^{NS} is an optionally substituted or protected nucleoside, e.g., those suitable protected for oligonucleotide synthesis. In some embodiments, N^{NS} is an oligonucleotide. In some embodiments, each nucleobase of N^{NS} (if any) is independently optionally protected, e.g., as suitable for oligonucleotide synthesis. In some embodiments, N^{NS} is connected to —O— through a linkage as described herein, e.g., a phosphate linkage. In some embodiments, an agent is or comprises

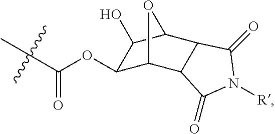

wherein R′ is as described herein. In some embodiments, an agent is or comprises

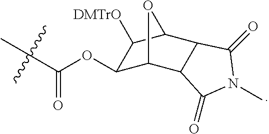

In some embodiments, an agent is or comprises

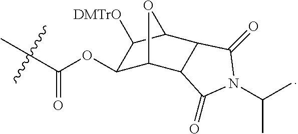

In some embodiments, an agent is or comprises

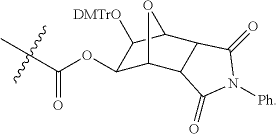

In some embodiments, an agent is or comprises

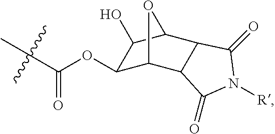

wherein R′ is as described herein. In some embodiments, R′ is optionally substituted C₁₋₆ aliphatic. In some embodiments, R′ is optionally substituted C₁₋₆ alkyl. In some embodiments, R′ is isopropyl. In some embodiments, R′ is methyl. In some embodiments, R′ is optionally substituted aryl. In some embodiments, R′ is optionally substituted phenyl. In some embodiments, R′ is phenyl. In some embodiments, the —OH reacts with a coupling partner, e.g., a phosphoramidite.

Various supports including those functionalized with linkers and/or loaded nucleosides are described in Example 1 as examples.

In some embodiments, R′ is independently —R, —C(O)R, —CO₂R, or —SO₂R, or two or more R′ are taken together with their intervening atoms to form an optionally substituted monocyclic, bicyclic or polycyclic, saturated, partially unsaturated, or aryl 3-30 membered ring having, in addition to the intervening atoms, 0-10 heteroatoms independently selected from oxygen, nitrogen, sulfur, phosphorus and silicon. In some embodiments, each R is independently hydrogen, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, a 5-30 membered heteroaryl ring having 1-10 heteroatoms independently selected from oxygen, nitrogen, sulfur, phosphorus and silicon, and a 3-30 membered heterocyclic ring having 1-10 heteroatoms independently selected from oxygen, nitrogen, sulfur, phosphorus and silicon.

In some embodiments, L^{SP} is an optionally substituted, linear or branched C₁-C₂₀ alkylene, wherein one or more methylene units of L^{SP} are optionally and independently replaced by the groups defined herein.

In some embodiments, L^{SP} is a covalent bond or an optionally substituted, linear or branched C₁-C₁₀ alkylene, wherein one or more methylene units of L^{SP} are optionally and independently replaced by the groups defined herein.

In some embodiments, L^{SP} is a covalent bond or an optionally substituted, linear or branched C20-C30 alkylene, wherein one or more methylene units of L^{SP} are optionally and independently replaced by the groups defined herein.

In some embodiments, one or more methylene units of L^{SP} is replaced by —N(R′)C(O)—, wherein R′ is H. In some embodiments, one or more methylene units of L^{SP} is replaced by —N(R′)C(O)—, wherein R′ is Me. In some embodiments, one or more methylene units of L^{SP} is replaced by —N(R′)C(O)—, wherein R′ is ethyl. In some embodiments, one or more methylene units of L^{SP} is replaced by —N(R′)C(O)—, wherein R′ is propyl. In some embodiments, one or more methylene units of L^{SP} is replaced by —N(R′)C(O)—, wherein R′ is isopropyl.

In some embodiments, one or more methylene units of O^{P} is replaced by —N(R′)C(O)O—, wherein R′ is H. In some embodiments, one or more methylene units of L^{SP} is replaced by —N(R′)C(O)O—, wherein R′ is Me. In some embodiments, one or more methylene units of L^{SP} is replaced by—N(R′)C(O)O—, wherein R′ is ethyl. In some embodiments, one or more methylene units of L^{SP} is replaced by —N(R′)C(O)O—, wherein R′ is propyl. In some embodiments, one or more methylene units of L^{SP} is replaced by —N(R′)C(O)O—, wherein R′ is isopropyl.

In some embodiments, one or more methylene units of L^{SP} is replaced by —N(R′)C(O)N(R′)—, wherein each R′ is H. In some embodiments, one or more methylene units of L^{SP} is replaced by—N(R′)C(O)N(R′)—, wherein each R′ is Me. In some embodiments, one or more methylene units of L^{SP} is replaced by —N(R′)C(O)N(R′)—, wherein each R′ is independently H or Me. In some embodiments, one or more methylene units of L^{SP} is replaced by —N(R′)C(O)N(R′)—, wherein each R′ is independently H, Me, or ethyl. In some embodiments, one or more methylene units of L^{SP} is replaced by —N(R′)C(O)N(R′)—, wherein each R′ is independently H, Me, ethyl, or propyl. In some embodiments, one or more methylene units of L^{SP} is replaced by —N(R′)C(O)N(R′)—, wherein each R′ is independently H, Me, ethyl, propyl, or isopropyl.

In some embodiments, one or more methylene units of L^{SP} is replaced by —O—. In some embodiments, one methylene unit of L^{SP} is replaced by —O—. In some embodiments, two methylene units oft L^{SP} are replaced by —O—. In some embodiments, three methylene units of L^{SP} are replaced by —O—. In some embodiments, four methylene units of L^{SP} are replaced by —O—. In some embodiments, five methylene units of L^{SP} are replaced by —O—. In some embodiments, six methylene units of L^{SP} are replaced by —O—. In some embodiments, seven methylene units of L^{SP} are replaced by —O—.

In some embodiments, one or more methylene units of L^{SP} is replaced by —C(O)—. In some embodiments, —C(O)— is connected to a nucleoside, e.g., to a 3′-carbon via oxygen.

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858.

### Biological Applications

As appreciated by those skilled in the art, oligonucleotides and compositions are useful for multiple purposes. In some embodiments, provided technologies (e.g., oligonucleotides, compositions, etc.) are useful for reducing levels, expression, activities, etc. of target nucleic acids (e.g., various transcripts) and products (e.g., mRNA, proteins, etc.) thereof. In some embodiments, provided technologies can be utilized for splicing modulation, e.g., exon skipping or inclusion. In some embodiments, provided technologies are useful for gene editing. As appreciated by those skilled in the art, provided oligonucleotides and compositions may function through one or more of a number of mechanism, e.g., RNase H pathway, RNAi, exon skipping, base/sequence editing, etc. As examples, certain properties and/or activities of a number of oligonucleotides and compositions are presented in the Examples. In some embodiments, an application is described in U.S. Pat. Nos. 9,394,333, 9,744,183, 9,605,019, 9,598,458, 9,982,257, 10,160,969, 10,4799,95, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858.

### Characterization and Assessment

In some embodiments, properties and/or activities of provided oligonucleotides and compositions can be characterized and/or assessed using various technologies available to those skilled in the art, e.g., biochemical assays (e.g., RNA cleavage assays, exon skipping assays), cell based assays, animal models, clinical trials, etc.

In some embodiments, properties and/or activities of oligonucleotides and compositions are compared to reference oligonucleotides and compositions thereof, respectively. In some embodiments, a reference oligonucleotide composition is a stereorandom oligonucleotide composition. In some embodiments, a reference oligonucleotide composition is a stereorandom composition of oligonucleotides of which all internucleotidic linkages are phosphorothioate. In some embodiments, a reference oligonucleotide composition is a DNA oligonucleotide composition with all phosphate linkages. In some embodiments, a reference oligonucleotide composition is otherwise identical to a provided chirally controlled oligonucleotide composition except that it is not chirally controlled. In some embodiments, a reference oligonucleotide composition is otherwise identical to a provided chirally controlled oligonucleotide composition except that it has a different pattern of stereochemistry. In some embodiments, a reference oligonucleotide composition is similar to a provided oligonucleotide composition except that it has a different modification of one or more sugar, base, and/or internucleotidic linkage, or pattern of modifications. In some embodiments, an oligonucleotide composition is stereorandom and a reference oligonucleotide composition is also stereorandom, but they differ in regards to sugar and/or base modification(s) or patterns thereof. In some embodiments, a reference composition is a composition of oligonucleotides having the same base sequence and the same chemical modifications. In some embodiments, a reference composition is a composition of oligonucleotides having the same base sequence and the same pattern of chemical modifications. In some embodiments, a reference composition is a non-chirally controlled (or stereorandom) composition of oligonucleotides having the same base sequence and chemical modifications. In some embodiments, a reference composition is a non-chirally controlled (or stereorandom) composition of oligonucleotides of the same constitution but is otherwise identical to a provided chirally controlled oligonucleotide composition. In some embodiments, a reference composition is a composition of oligonucleotides having the same base sequence but different chemical modifications, including but not limited to chemical modifications described herein. In some embodiments, a reference composition is a composition of oligonucleotides having the same base sequence but different patterns of internucleotidic linkages and/or stereochemistry of internucleotidic linkages and/or chemical modifications.

Various methods are known in the art for detection of gene products, the expression, level and/or activity of which may be altered after introduction or administration of a provided oligonucleotide. For example, transcripts and their variants in which an exon is skipped can be detected and quantified with qPCR, and protein levels can be determined via Western blot.

In some embodiments, assessment of efficacy of oligonucleotides can be performed in biochemical assays or in vitro in cells. In some embodiments, provided oligonucleotides can be introduced to cells via various methods available to those skilled in the art, e.g., gymnotic delivery, transfection, lipofection, etc.

Certain useful technologies are described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858.

### Pharmaceutical Compositions

In some embodiments, the present disclosure provides pharmaceutical compositions comprising a provided compound, e.g., an oligonucleotide, or a pharmaceutically acceptable salt thereof, and a pharmaceutical carrier. In some embodiments, for therapeutic and clinical purposes, oligonucleotides of the present disclosure are provided as pharmaceutical compositions. In some embodiments, pharmaceutical compositions are chirally controlled oligonucleotide compositions.

As appreciated by those skilled in the art, oligonucleotides of the present disclosure can be provided in their acid, base or salt forms. In some embodiments, oligonucleotides can be in acid forms, e.g., for natural phosphate linkages, in the form of —OP(O)(OH)O—; for phosphorothioate internucleotidic linkages, in the form of —OP(O)(SH)O—; etc. In some embodiments, provided oligonucleotides can be in salt forms, e.g., for natural phosphate linkages, in the form of —OP(O)(ONa)O— in sodium salts; for phosphorothioate internucleotidic linkages, in the form of —OP(O)(SNa)O— in sodium salts; etc. Unless otherwise noted, oligonucleotides of the present disclosure can exist in acid, base and/or salt forms.

In some embodiments, the pharmaceutical composition is formulated for intravenous injection, oral administration, buccal administration, inhalation, nasal administration, topical administration, ophthalmic administration or otic administration. In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

In some embodiments, the present disclosure provides salts of oligonucleotides and pharmaceutical compositions thereof. In some embodiments, a salt is a pharmaceutically acceptable salt. In some embodiments, a pharmaceutical composition comprises an oligonucleotide, optionally in its salt form, and a sodium salt. In some embodiments, a pharmaceutical composition comprises an oligonucleotide, optionally in its salt form, and sodium chloride. In some embodiments, each hydrogen ion of an oligonucleotide that may be donated to a base (e.g., under conditions of an aqueous solution, a pharmaceutical composition, etc.) is replaced by a non-Ft cation. For example, in some embodiments, a pharmaceutically acceptable salt of an oligonucleotide is an all-metal ion salt, wherein each hydrogen ion (for example, of —OH, —SH, etc.) of each internucleotidic linkage (e.g., a natural phosphate linkage, a phosphorothioate internucleotidic linkage, etc.) is replaced by a metal ion. Various suitable metal salts for pharmaceutical compositions are widely known in the art and can be utilized in accordance with the present disclosure. In some embodiments, a pharmaceutically acceptable salt is a sodium salt. In some embodiments, a pharmaceutically acceptable salt is magnesium salt. In some embodiments, a pharmaceutically acceptable salt is a calcium salt. In some embodiments, a pharmaceutically acceptable salt is a potassium salt. In some embodiments, a pharmaceutically acceptable salt is an ammonium salt (cation N(R)₄⁺). In some embodiments, a pharmaceutically acceptable salt comprises one and no more than one types of cation. In some embodiments, a pharmaceutically acceptable salt comprises two or more types of cation. In some embodiments, a cation is Li⁺, Na⁺, K⁺, Mg²⁺or Ca²⁺. In some embodiments, a pharmaceutically acceptable salt is an all-sodium salt. In some embodiments, a pharmaceutically acceptable salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate internucleotidic linkage linkage (acid form —O—P(O)(SH)—O—), if any, exists as its sodium salt form (—O—P(O)(SNa)—O—).

Various technologies for delivering nucleic acids and/or oligonucleotides are known in the art can be utilized in accordance with the present disclosure. For example, a variety of supramolecular nanocarriers can be used to deliver nucleic acids. Example nanocarriers include, but are not limited to liposomes, cationic polymer complexes and various polymeric compounds. Complexation of nucleic acids with various polycations is another approach for intracellular delivery; this includes use of PEGylated polycations, polyethyleneamine (PEI) complexes, cationic block co-polymers, and dendrimers. Several cationic nanocarriers, including PEI and polyamidoamine dendrimers help to release contents from endosomes. Other approaches include use of polymeric nanoparticles, microspheres, liposomes, dendrimers, biodegradable polymers, conjugates, prodrugs, inorganic colloids such as sulfur or iron, antibodies, implants, biodegradable implants, biodegradable microspheres, osmotically controlled implants, lipid nanoparticles, emulsions, oily solutions, aqueous solutions, biodegradable polymers, poly(lactide-coglycolic acid), poly(lactic acid), liquid depot, polymer micelles, quantum dots and lipoplexes. In some embodiments, an oligonucleotide is conjugated to another molecule.

In therapeutic and/or diagnostic applications, compounds, e.g., oligonucleotides, of the disclosure can be formulated for a variety of modes of administration, including systemic and topical or localized administration. Techniques and formulations generally may be found in Remington, The Science and Practice of Pharmacy (20th ed. 2000).

Depending on the specific conditions, disorders or diseases being treated, provided agents, e.g., oligonucleotides, may be formulated into liquid or solid dosage forms and administered systemically or locally. Provided oligonucleotides may be delivered, for example, in a timed- or sustained- low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or another mode of delivery.

For injection, provided agents, e.g., oligonucleotides may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulations. Such penetrants are generally known in the art and can be utilized in accordance with the present disclosure.

Provided compounds, e.g., oligonucleotides, can be formulated readily using pharmaceutically acceptable carriers well known in the art into dosages suitable for oral administration. In some embodiments such carriers enable provided oligonucleotides to be formulated as tablets, pills, capsules, liquids, gels, syrups, slurries, suspensions and the like, for, e.g., oral ingestion by a subject (e.g., patient) to be treated.

For nasal or inhalation delivery, provided compounds, e.g., oligonucleotides, may be formulated by methods known to those of skill in the art, and may include, e.g., examples of solubilizing, diluting, or dispersing substances such as saline, preservatives, such as benzyl alcohol, absorption promoters, and fluorocarbons.

In certain embodiments, oligonucleotides and compositions are delivered to the CNS. In certain embodiments, oligonucleotides and compositions are delivered to the cerebrospinal fluid. In certain embodiments, oligonucleotides and compositions are administered to the brain parenchyma. In certain embodiments, oligonucleotides and compositions are delivered to an animal/subject by intrathecal administration, or intracerebroventricular administration. Broad distribution of oligonucleotides and compositions may be achieved with methods of administration described herein and/or known in the art.

In certain embodiments, parenteral administration is by injection, by, e.g., a syringe, a pump, etc. In certain embodiments, an injection is a bolus injection. In certain embodiments, an injection is administered directly to a tissue or location, such as striatum, caudate, cortex, hippocampus and/or cerebellum.

Pharmaceutical compositions suitable for use in the present disclosure include compositions wherein the active ingredients, e.g., oligonucleotides, are contained in effective amounts to achieve their intended purposes. Determination of the effective amounts is well within the capability of those skilled in the art, especially in light of the detailed disclosure provided herein.

In addition to active ingredients, pharmaceutical compositions may contain suitable pharmaceutically acceptable carriers comprising excipients and auxiliaries which facilitate processing of an active compound into preparations which can be used pharmaceutically. Preparations formulated for oral administration may be in the form of tablets, dragees, capsules, or solutions.

In some embodiments, pharmaceutical compositions for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

In some embodiments, dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. Push-fit capsules can contain active ingredients, e.g., oligonucleotides, in admixture with fillers such as lactose, binders such as starches, and/or lubricants such as talc or magnesium stearate and, optionally, stabilizers. In soft capsules, active compounds, e.g., oligonucleotides, may be dissolved or suspended in suitable liquids, such as fatty oils, liquid paraffin, or liquid polyethylene glycols (PEGs). In addition, stabilizers may be added.

In some embodiments, a provided composition comprises a lipid. In some embodiments, a lipid is conjugated to an active compound, e.g., an oligonucleotide. In some embodiments, a lipid is not conjugated to an active compound. In some embodiments, a lipid comprises a C₁₀-C₄₀ linear, saturated or partially unsaturated, aliphatic chain. In some embodiments, a lipid comprises a C₁₀-C₄₀ linear, saturated or partially unsaturated, aliphatic chain, optionally substituted with one or more C₁₋₄ aliphatic group. In some embodiments, the lipid is selected from the group consisting of lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol. In some embodiments, an active compound is a provided oligonucleotide. In some embodiments, a composition comprises a lipid and an an active compound, and further comprises another component which is another lipid or a targeting compound or moiety. In some embodiments, a lipid is an amino lipid; an amphipathic lipid; an anionic lipid; an apolipoprotein; a cationic lipid; a low molecular weight cationic lipid; a cationic lipid such as CLinDMA and DLinDMA; an ionizable cationic lipid; a cloaking component; a helper lipid; a lipopeptide; a neutral lipid; a neutral zwitterionic lipid; a hydrophobic small molecule; a hydrophobic vitamin; a PEG-lipid; an uncharged lipid modified with one or more hydrophilic polymers; phospholipid; a phospholipid such as 1,2-dioleoyl-sn-glycero-3-phosphoethanolamine; a stealth lipid; a sterol; a cholesterol; a targeting lipid; or another lipid described herein or reported in the art suitable for pharmaceutical uses. In some embodiments, a composition comprises a lipid and a portion of another lipid capable of mediating at least one function of another lipid. In some embodiments, a targeting compound or moiety is capable of targeting a compound (e.g., an oligonucleotide) to a particular cell or tissue or subset of cells or tissues. In some embodiments, a targeting moiety is designed to take advantage of cell- or tissue-specific expression of particular targets, receptors, proteins, or another subcellular component. In some embodiments, a targeting moiety is a ligand (e.g., a small molecule, antibody, peptide, protein, carbohydrate, aptamer, etc.) that targets a composition to a cell or tissue, and/or binds to a target, receptor, protein, or another subcellular component.

Certain example lipids for delivery of an active compound, e.g., an oligonucleotide, allow (e.g., do not prevent or interfere with) the function of an active compound. In some embodiments, a lipid is lauric acid, myristic acid, palmitic acid, stearic acid, oleic acid, linoleic acid, alpha-linolenic acid, gamma-linolenic acid, docosahexaenoic acid (cis-DHA), turbinaric acid or dilinoleyl alcohol.

As described in the present disclosure, lipid conjugation, such as conjugation with fatty acids, may improve one or more properties of oligonucleotides.

In some embodiments, a composition for delivery of an active compound, e.g., an oligonucleotide, is capable of targeting an active compound to particular cells or tissues as desired. In some embodiments, a composition for delivery of an active compound is capable of targeting an active compound to a muscle cell or tissue. In some embodiments, the present disclosure provides compositions and methods related to delivery of active compounds, wherein the compositions comprise an active compound and a lipid. In various embodiments to a muscle cell or tissue, a lipid is selected from lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol.

In some embodiments, an oligonucleotide is delivered via a composition comprising any one or more of, or a method of delivery involving the use of any one or more of: transferrin receptor-targeted nanoparticle; cationic liposome-based delivery strategy; cationic liposome; polymeric nanoparticle; viral carrier; retrovirus; adeno-associated virus; stable nucleic acid lipid particle; polymer; cell-penetrating peptide; lipid; dendrimer; neutral lipid; cholesterol; lipid-like molecule; fusogenic lipid; hydrophilic molecule; polyethylene glycol (PEG) or a derivative thereof; shielding lipid; PEGylated lipid; PEG-C-DMSO; PEG-C-DMSA; DSPC; ionizable lipid; a guanidinium-based cholesterol derivative; ion-coated nanoparticle; metal-ion coated nanoparticle; manganese ion-coated nanoparticle; angubindin-1; nanogel; incorporation of an oligonucleotide into a branched nucleic acid structure; and/or incorporation of an oligonucleotide into a branched nucleic acid structure comprising 2, 3, 4 or more oligonucleotides.

In some embodiments, a composition comprising an oligonucleotide is lyophilized. In some embodiments, a composition comprising an oligonucleotide is lyophilized, and the lyophilized oligonucleotide is in a vial. In some embodiments, the vial is back filled with nitrogen. In some embodiments, the lyophilized oligonucleotide composition is reconstituted prior to administration. In some embodiments, the lyophilized oligonucleotide composition is reconstituted with a sodium chloride solution prior to administration. In some embodiments, the lyophilized oligonucleotide composition is reconstituted with a 0.9% sodium chloride solution prior to administration. In some embodiments, reconstitution occurs at the clinical site for administration. In some embodiments, in a lyophilized composition, an oligonucleotide composition is chirally controlled or comprises at least one chirally controlled internucleotidic linkage and/or the oligonucleotide.

In some embodiments, oligonucleotides and compositions are formulated and/or administrated as described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the formulation and administration technologies are independently incorporated herein by reference.

Among other things, the present disclosure provides the following Embodiments:
1. An oligonucleotide having the structure of:

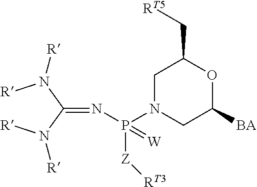

or a salt thereof, wherein:

BA is an optionally substituted or protected nucleobase;

R^{T5} is optionally substituted or protected hydroxyl, an optionally substituted or protected nucleotide moiety, an oligonucleotide moiety, R′, or an additional chemical moiety optionally connected through a linker;

R^{T3} is hydrogen, an optionally substituted or protected or nucleoside nucleotide moiety, an oligonucleotide moiety, R′, or an additional chemical moiety optionally connected through a linker;

W is O, S or Se;

Z is —O—, —S—, N(R′)—;

each R^{L} is independently -L^{L}-R′ or —N═C(-L^{L}-R′)₂;

Ring A^{s} is an optionally substituted 3-30 membered, monocyclic, bicyclic or polycyclic ring having, in addition to the nitrogen, 0-10 heteroatoms;

each of L^{s}, L^{L1}, L^{L2} and L^{L} is independently L;

-Cy^{IL}- is -Cy-;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—,a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′))O——OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R; each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
2. An oligonucleotide having the structure of:

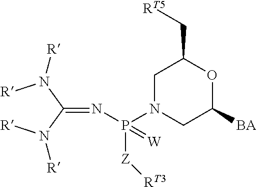

or a salt thereof, wherein:

BA is an optionally substituted or protected nucleobase;

R^{T5} is optionally substituted or protected hydroxyl, an optionally substituted or protected nucleotide moiety, an oligonucleotide moiety, R′, or an additional chemical moiety optionally connected through a linker;

R^{T3} is hydrogen, an optionally substituted or protected or nucleoside nucleotide moiety, an oligonucleotide moiety, R′, or an additional chemical moiety optionally connected through a linker;

W is O, N(-L^{L}-R^{L}), S or Se;

Z is —O—, —S—, —N(L^{L}-R^{L}) , or L^{L};

each R^{L} is independently -L^{L}-R′ or —N═C(-L^{L}-R′)₂;

Ring A^{s} is an optionally substituted 3-30 membered, monocyclic, bicyclic or polycyclic ring having, in addition to the nitrogen, 0-10 heteroatoms;

each of L′, L^{L1}, L^{L2} and L^{L} is independently L;

-Cy^{IL}- is -Cy-;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′))O——OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)_{3]0}—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
3. The oligonucleotide of any one of the preceding Embodiments, wherein the oligonucleotide has the structure of

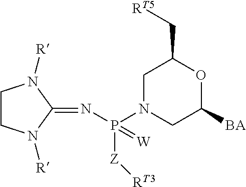

or a salt thereof.
4. An oligonucleotide, wherein the oligonucleotide has the structure of:

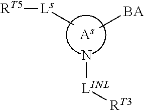

or a salt thereof, wherein:

BA is an optionally substituted or protected nucleobase;

R^{T5} is optionally substituted or protected hydroxyl, an optionally substituted or protected nucleotide moiety, an oligonucleotide moiety, R′, or an additional chemical moiety optionally connected through a linker;

R^{T3} is hydrogen, an optionally substituted or protected or nucleoside nucleotide moiety, an oligonucleotide moiety, R′, or an additional chemical moiety optionally connected through a linker;

L^{INL} is —Y—P^{L}(—X—R^{L})—Z—, —C(O)—O— wherein —C(O)— in bonded to a nitrogen atom,

—C(O)—N(R′)—, or -L^{L1}-Cy^{IL}-L^{L2}-,

P^{L} is P, P(═W), P->B(_{L}-R^{L})₃, or P^{N};

W is O, N(-L^{L}-R^{L}), S or Se;

P^{N} is P═N—C(-L^{L}-R′)(=L^{N}-R′) or P═N-L^{L}-R^{L};

L^{N} is ═N-L^{L1}-, ═CH-L^{L1}- wherein CH is optionally substituted, or ═N⁺(R′)(Q⁻)-L^{L1}-;

Q⁻ is an anion;

each of X, Y and Z is independently —O—, —S—, —N(-L^{L}-R^{L})—, or L^{L};

each R^{L} is independently -L^{L}-R′ or —N═C(-L^{L}-R′)₂;

Ring A^{s} is an optionally substituted 3-30 membered, monocyclic, bicyclic or polycyclic ring having, in addition to the nitrogen, 0-10 heteroatoms;

each of L^{s}, L^{L1}, L^{L2} and L^{L} is independently L;

-Cy^{IL}- is -Cy-;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)O—, —OP(SR′)—, —OP(NR′)—, —OP(R′)—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
5. The oligonucleotide of any one of the preceding Embodiments, wherein R^{T5} is —OH.
6. The oligonucleotide of any one of Embodiments 1-4, wherein R^{T5} is protected hydroxyl.
7. The oligonucleotide of any one of Embodiments 1-4, wherein R^{T5} is —ODMTr.
8. The oligonucleotide of any one of Embodiments 1-4, wherein R^{T5} is R′.
9. The oligonucleotide of any one of Embodiments 1-4, wherein R^{T5} is an optionally substituted or protected nucleotide moiety.
10. The oligonucleotide of any one of Embodiments 1-4, wherein R^{T5} is an additional chemical moiety optionally connected through a linker.
11. The oligonucleotide of Embodiment 10, wherein R^{T5} comprises one or more ASGPR ligand.
12. The oligonucleotide of Embodiment 10, wherein R^{T5} comprises one or more GalNAc.
13. The oligonucleotide of any one of Embodiments 1-4, wherein R^{T5} is an oligonucleotide moiety.
14. The oligonucleotide of Embodiment 13, wherein R^{T5} is an oligonucleotide moiety comprising one or more modified sugars.
15. The oligonucleotide of any one of Embodiments 13-14, wherein R^{T5} is an oligonucleotide moiety comprising one or more modified nucleobases.
16. The oligonucleotide of any one of Embodiments 13-15, wherein R^{T5} is an oligonucleotide moiety comprising one or more modified internucleotidic linkages.
17. The oligonucleotide of any one of Embodiments 13-15, wherein R^{T5} is an oligonucleotide moiety comprising one or more chirally controlled modified internucleotidic linkages.
18. The oligonucleotide of any one of Embodiments 13-17, wherein R^{T5} is an oligonucleotide moiety comprising one or more chirally controlled phosphorothioate internucleotidic linkages.
19. The oligonucleotide of any one of Embodiments 13-18, wherein R^{T5} is an oligonucleotide moiety comprising one or more non-negatively charged internucleotidic linkages.
20. The oligonucleotide of any one of Embodiments 13-19, wherein R^{T5} is an oligonucleotide moiety comprising one or more chirally controlled non-negatively charged internucleotidic linkages.
21. The oligonucleotide of any one of the preceding Embodiments, wherein R^{T3} is hydrogen.
22. The oligonucleotide of any one of the preceding Embodiments, wherein R^{T3} is R′.
23. The oligonucleotide of any one of Embodiments 1-20, wherein R^{T3} is an optionally substituted or protected nucleotide moiety.
24. The oligonucleotide of any one of Embodiments 1-20, wherein R^{T3} is an optionally substituted or protected nucleoside moiety.
25. The oligonucleotide of any one of Embodiments 1-20, wherein R^{T3} is an additional chemical moiety optionally connected through a linker.
26. The oligonucleotide of Embodiment 25, wherein R^{T3} comprises one or more ASGPR ligand.
27. The oligonucleotide of Embodiment 25, wherein R^{T3} comprises one or more GalNAc.
28. The oligonucleotide of any one of Embodiments 1-20, wherein R^{T3} is an oligonucleotide moiety.
29. The oligonucleotide of Embodiment 28, wherein R^{T3} is an oligonucleotide moiety comprising one or more modified sugars.
30. The oligonucleotide of any one of Embodiments 28-29, wherein R^{T3} is an oligonucleotide moiety comprising one or more modified nucleobases.
31. The oligonucleotide of any one of Embodiments 28-30, wherein R^{T3} is an oligonucleotide moiety comprising one or more modified internucleotidic linkages.
32. The oligonucleotide of any one of Embodiments 28-30, wherein R^{T3} is an oligonucleotide moiety comprising one or more chirally controlled modified internucleotidic linkages.
33. The oligonucleotide of any one of Embodiments 28-32, wherein R^{T3} is an oligonucleotide moiety comprising one or more chirally controlled phosphorothioate internucleotidic linkages.
34. The oligonucleotide of any one of Embodiments 28-33, wherein R^{T3} is an oligonucleotide moiety comprising one or more non-negatively charged internucleotidic linkages.
35. The oligonucleotide of any one of Embodiments 28-34, wherein R^{T3} is an oligonucleotide moiety comprising one or more chirally controlled non-negatively charged internucleotidic linkages.
36. The oligonucleotide of any one of Embodiments 23-35, wherein the nucleoside moiety, the nucleotide moiety, the additional chemical moiety, or the oligonucleotide moiety is connected to a support optionally through a linker.
37. The oligonucleotide of Embodiment 36, wherein the support is a solid support suitable for oligonucleotide synthesis.
38. An oligonucleotide, comprising:

one or more sugar units independently selected from:
a sugar having the structure of:

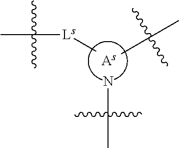

and an acyclic sugar, or

one or more modified internucleotidic linkages each independently having the structure of:
—Y—P^{L}(—X—R^{L})—Z—,
—C(O)—O— wherein —C(O)— in bonded to a nitrogen atom,
—C(O)—N(R′)—, or
-L^{L1}-Cy^{IL}-L^{L2}-,
   wherein:

P^{L} is P, P(═W), P->B(-L^{L}-R^{L})₃, or P^{N};

W is O, N(-L^{L}-R^{L}), S or Se;

P^{N} is P═N—C(-L^{L}-R′)(=L^{N}-R′) or P═N-L^{L}-R^{L};

L^{N} is ═N-L^{L1}-, ═CH-L^{L1}- wherein CH is optionally substituted, or ═N⁺(R′)(Q⁻)-L^{L1}-;

Q⁻ is an anion;

each of X, Y and Z is independently —O—, —S—, —N(-L^{L}-R^{L})-, or L^{L},

each R^{L} is independently -L^{L}-R′ or —N═C(-L^{L}-R′)₂;

Ring A^{s} is an optionally substituted 3-30 membered, monocyclic, bicyclic or polycycling ring having, in addition to the nitrogen, 0-10 heteroatoms;

each of L^{s}, L^{L1}, L^{L2} and L^{L} is independently L;

-Cy^{IL}- is -Cy-;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)——P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)—, —OP(SR′)—, —OP(NR′)—, —OP(R′)—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R; each R is independently —H, or an optionally substituted group selected from C₁₋₃₀ aliphatic, C₁-₃₀ heteroaliphatic having 1-10 heteroatoms, C₆-₃₀ aryl, C₆-₃₀ arylaliphatic, C₆-₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
39. The oligonucleotide of any one of the preceding Embodiments, comprising a nucleoside unit comprising a morpholine unit, wherein the nitrogen of the morpholine unit is bonded to an internucleotidic linkage having the structure of —P(═W)(—N═C[N(R′)₂]₂)—O—.
40. The oligonucleotide of any one of the preceding Embodiments, wherein an occurrence of P^{L} is P(═O).
41. The oligonucleotide of any one of the preceding Embodiments, wherein an occurrence of P^{L} is P(═S).
42. The oligonucleotide of any one of the preceding Embodiments, wherein an occurrence of P^{L} is P^{N}.
43. The oligonucleotide of any one of the preceding Embodiments, wherein an occurrence of P^{L} is P═N—C(L^{L}-R′)(=L^{N}-R′).
44. The oligonucleotide of any one of the preceding Embodiments, wherein an occurrence of P^{L} is P═N-L^{L}-R^{L}.
45. The oligonucleotide of any one of the preceding Embodiments, wherein an occurrence of Y is a covalent bond.
46. The oligonucleotide of any one of the preceding Embodiments, wherein an occurrence of Y is —O—.
47. The oligonucleotide of any one of the preceding Embodiments, wherein the oligonucleotide comprises a nucleoside unit comprising a morpholine unit, wherein the nucleoside unit has the structure of

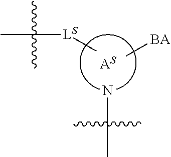

or a salt form thereof, wherein BA is a nucleobase, and N is bond to an internucleotidic linkage.
48. The oligonucleotide of any one of the preceding Embodiments, wherein the oligonucleotide comprises one or more internucleotidic linkages each independently having the structure of —Y—P(═W)(—X-L^{L}-R^{L})—Z—, wherein Y is —O—.
49. The oligonucleotide of any one of the preceding Embodiments, wherein the oligonucleotide comprises one or more internucleotidic linkages each independently having the structure of —P(═W)(—X-L^{L}-R^{L})—Z—.
50. The oligonucleotide of any one of the preceding Embodiments, wherein the oligonucleotide comprises a nucleoside unit comprising a morpholine unit, wherein the nucleoside unit has the structure of

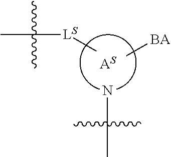

or a salt form thereof, wherein BA is a nucleobase, the N is bond to the P of an internucleotidic linkage having the structure of —P^{L}(—X—R^{L})—Z—.
51. The oligonucleotide of any one of the preceding Embodiments, wherein the oligonucleotide comprises a nucleoside unit comprising a morpholine unit, wherein the nucleoside unit has the structure of

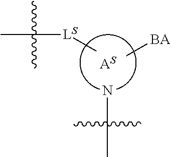

or a salt form thereof, wherein BA is a nucleobase, the N is bond to the P of an internucleotidic linkage having the structure of —P(═W)(—X-L^{L}-R^{L})—Z—.
52. The oligonucleotide of any one of the preceding Embodiments, wherein the oligonucleotide comprises a nucleoside unit comprising a morpholine unit, wherein the nucleoside unit has the structure of

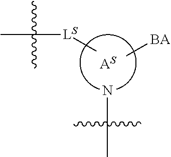

or a salt form thereof, wherein BA is a nucleobase, the N is bond to —C(O)—O—, wherein the —C(O)— is bonded to N.
53. The oligonucleotide of any one of the preceding Embodiments, wherein each Z is —O—.
54. The oligonucleotide of any one of the preceding Embodiments, wherein an occurrence of W is O.
55. The oligonucleotide of any one of the preceding Embodiments, wherein an occurrence of W is S.
56. The oligonucleotide of any one of Embodiments 1-54, wherein each W is O.
57. The oligonucleotide of any one of Embodiments 48-56, wherein an occurrence of X is O.
58. The oligonucleotide of Embodiment 57, wherein an occurrence of —X—R^{L} is —OH.
59. The oligonucleotide of any one of Embodiments 48-56, wherein an occurrence of X is S.
60. The oligonucleotide of Embodiment 59, wherein an occurrence of —X—R^{L} is —SH.
61. The oligonucleotide of any one of Embodiments 48-56, wherein an occurrence of X is a covalent bond.
62. The oligonucleotide of any one of the preceding Embodiments, wherein an occurrence of -L^{L}-R^{L} is —N(R′)₂.
63. The oligonucleotide of any one of the preceding Embodiments, wherein an occurrence of -L^{L}-R^{L} is —N(CH₃)₂.
64. The oligonucleotide of any one of Embodiments 1-61, wherein an occurrence of R^{L} is —N═C(— L^{L}-R′)₂.
65. The oligonucleotide of Embodiment 64, wherein an occurrence of -L^{L}- is —N(R′)—.
66. The oligonucleotide of Embodiment 64 or 65, wherein R^{L} is —N═C[N(R′)₂]₂.
67. The oligonucleotide of Embodiment 66, wherein two R′ on the same nitrogen are taken together with their intervening atoms to form an optionally substituted, 3-30 membered, monocyclic, bicyclic or polycyclic ring having, in addition to the intervening atoms, 0-10 heteroatoms.
68. The oligonucleotide of Embodiment 66, wherein two R′ on the first nitrogen are taken together with their intervening atoms to form an optionally substituted, 3-30 membered, monocyclic, bicyclic or polycyclic ring having, in addition to the intervening atoms, 0-10 heteroatoms, and two R′ on the second nitrogen are taken together with their intervening atoms to form an optionally substituted, 3-30 membered, monocyclic, bicyclic or polycyclic ring having, in addition to the intervening atoms, 0-10 heteroatoms.
69. The oligonucleotide of Embodiment 66, wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms.
70. The oligonucleotide of Embodiment 66, wherein one R′ on one N and one R′ or the other N are taken together with their intervening atoms to form an optionally substituted, 5-membered, monocyclic, bicyclic or polycyclic ring having, in addition to the intervening atoms, 0 heteroatoms.
71. The oligonucleotide of any one of Embodiments 67-70, wherein the formed ring is saturated.
72. The oligonucleotide of any one of Embodiments 61-71, wherein R^{L} is

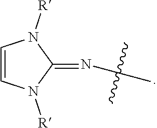

73. The oligonucleotide of any one of Embodiments 61-74, wherein R^{L} is

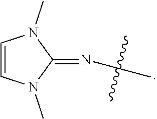 [SMILES: *N=c1n(C)ccn1C]

74. The oligonucleotide of any one of Embodiments 61-71, wherein R^{L} is

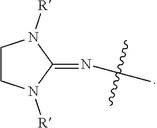

75. The oligonucleotide of any one of Embodiments 61-74, wherein R^{L} is

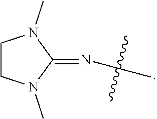 [SMILES: *N=C1N(C)CCN1C]

76. The oligonucleotide of any one of the preceding Embodiments, wherein the oligonucleotide comprises one or more internucleotidic linkages each independently having the structure of —C(O)—O—, wherein —C(O)— is bonded to a nitrogen atom.
77. The oligonucleotide of any one of the preceding Embodiments, wherein the oligonucleotide comprises a nucleoside unit comprising a morpholine unit, wherein the nucleoside unit has the structure of

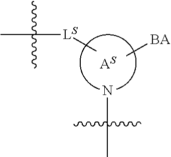

or a salt form thereof, wherein BA is a nucleobase, the N is bond to —C(O)— of an internucleotidic linkage having the structure of —C(O)—O—.
78. The oligonucleotide of any one of the preceding Embodiments, wherein the oligonucleotide comprises a nucleoside unit comprising a morpholine unit, wherein the nucleoside unit has the structure of

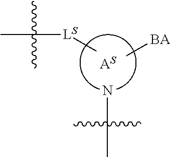

or a salt form thereof, wherein BA is a nucleobase, the N is bond to —C(O)— of an internucleotidic linkage having the structure of —C(O)—N(R′)—.
79. The oligonucleotide of any one of the preceding Embodiments, wherein

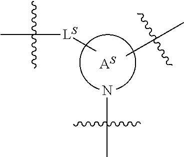

is optionally substituted

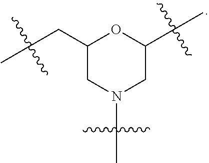 [SMILES: *CC1CN(*)CC(*)O1]

80. The oligonucleotide of any one of the preceding Embodiments, wherein

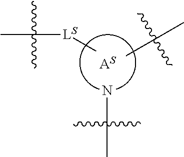

is optionally substituted

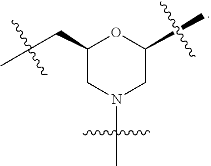

81. The oligonucleotide of any one of the preceding Embodiments, wherein

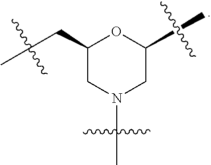

82. The oligonucleotide of any one of the preceding Embodiments, wherein an occurrence of

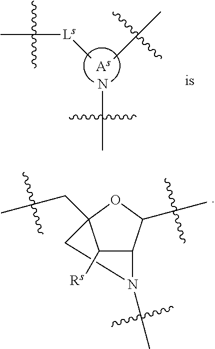

83. The oligonucleotide of any one of the preceding Embodiments, wherein an occurrence of

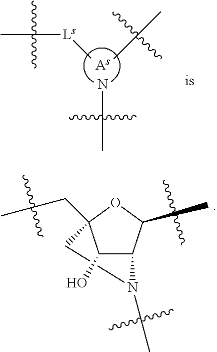

84 The oligonucleotide of any one of the preceding Embodiments, wherein an occurrence of

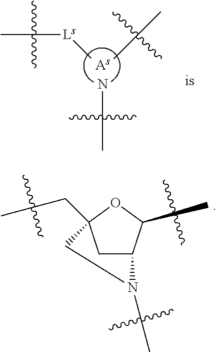

85. The oligonucleotide of any one of Embodiments 1-84, wherein the -L^{s}- or —CH₂— is bonded to an internucleotidic linkage.
86. The oligonucleotide of any one of the preceding Embodiments, wherein the oligonucleotide comprises one or more (e.g., 1, 2, 3, 4, 5, 6, 7, 8, 9, 10) natural phosphate linkages.
87. The oligonucleotide of any one of the preceding Embodiments, wherein the oligonucleotide comprises one or more (e.g., 1, 2, 3, 4, 5, 6, 7, 8, 9, 10, 11, 12, 13, 14, 15, 16, 17, 18, 19, 20) phosphorothioate internucleotidic linkages.
88. The oligonucleotide of any one of the preceding Embodiments, wherein the oligonucleotide comprises 2-200 nucleobases.
89. The oligonucleotide of any one of the preceding Embodiments, wherein the oligonucleotide comprises 15-50 nucleobases.
90. The oligonucleotide of any one of the preceding Embodiments, wherein the oligonucleotide comprises about 15 or more nucleobases.
91. The oligonucleotide of any one of the preceding Embodiments, wherein the oligonucleotide comprises about 20 or more nucleobases.
92. The oligonucleotide of any one of the preceding Embodiments, wherein one or more internucleotidic linkages each independently comprise a stereodefined linkage phosphorus in the Sp configuration.
93. The oligonucleotide of any one of the preceding Embodiments, wherein at least 60%, 70%, 75%, 80%, 85%, 90% or 95% of all chiral internucleotidic linkages independently chirally controlled.
94. The oligonucleotide of any one of the preceding Embodiments, wherein at least 60%, 70%, 75%, 80%, 85%, 90% or 95% of all chirally controlled phosphorothioate internucleotidic linkages are Sp.
95. The oligonucleotide of any one of the preceding Embodiments, wherein at least 60%, 70%, 75%, 80%, 85%, 90% or 95% of all chirally controlled non-negatively charged internucleotidic linkages are Rp.
96. The oligonucleotide of any one of the preceding Embodiments, wherein at least 60%, 70%, 75%, 80%, 85%, 90% or 95% of all chirally controlled internucleotidic linkages are Sp.
97. The oligonucleotide of any one of the preceding Embodiments, wherein at least 60%, 70%, 75%, 80%, 85%, 90% or 95% of all modified internucleotidic linkages are phosphorothioate internucleotidic linkages.
98. The oligonucleotide of any one of the preceding Embodiments, wherein at least 60%, 70%, 75%, 80%, 85%, 90% or 95% of all modified internucleotidic linkages are phosphorothioate internucleotidic linkages having a Sp configuration.
99. The oligonucleotide of any one of the preceding Embodiments, wherein at least 60%, 70%, 75%, 80%, 85%, 90% or 95% of all internucleotidic linkages are phosphorothioate internucleotidic linkages.
100. The oligonucleotide of any one of the preceding Embodiments, wherein at least 60%, 70%, 75%, 80%, 85%, 90% or 95% of all internucleotidic linkages are phosphorothioate internucleotidic linkages having a Sp configuration.
101. The oligonucleotide of any one of the preceding Embodiments, wherein each phosphorothioate internucleotidic linkage is independently chirally controlled.
102. The oligonucleotide of any one of the preceding Embodiments, wherein the pattern of backbone chiral centers comprises [(Rp/Op)n(Sp)m]y, wherein each of n, m, and y is independently 1-50, and each Np is independently Rp or Sp.
103. The oligonucleotide of any one of the preceding Embodiments, wherein the pattern of backbone chiral centers comprises (Np)t[(Rp/Op)n(Sp)m]y, wherein each oft, n, m, and y is independently 1-50, and each Np is independently Rp or Sp.
104. The oligonucleotide of any one of the preceding Embodiments, wherein the pattern of backbone chiral centers comprises (Sp)t[(Rp/Op)n(Sp)m]y, wherein each oft, n, m, and y is independently 1-50.
105. The oligonucleotide of any one of the preceding Embodiments, wherein each Op indicates a linkage phosphorus being achiral in a natural phosphate linkage.
106. The oligonucleotide of any one of the preceding Embodiments, wherein the oligonucleotide comprises a modified sugar.
107. The oligonucleotide of any one of the preceding Embodiments, comprising one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) sugars independently having the structure of

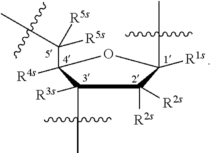

108. The oligonucleotide of Embodiment 107, wherein an occurrence of R⁵s is —CH₃.
109. The oligonucleotide of any one of Embodiments 107-108, wherein in one an occurrence of sugar one R^{5s} is —CH₃, and the other is —H.
110. The oligonucleotide of Embodiment 109, wherein the 5′-carbon is R.
111. The oligonucleotide of Embodiment 109, wherein the 5′-carbon is S.
112. The oligonucleotide of any one of Embodiments 107-111, wherein an occurrence of R^{5s} is —H.
113. The oligonucleotide of any one of Embodiments 107-112, wherein an occurrence of R^{4s} is —H.
114. The oligonucleotide of any one of Embodiments 107-112, wherein each occurrence of R^{4s} is independently —H, or is taken together with a R^{2s} to form a bridge having the structure of -L^{b}-L^{b}-, wherein each L^{b} is independently L.
115. The oligonucleotide of any one of Embodiments 107-114, wherein an occurrence of R^{3s} is —H.
116. The oligonucleotide of any one of Embodiments 107-114, wherein each occurrence of R^{3s} is —H.
117. The oligonucleotide of any one of Embodiments 107-116, wherein an occurrence of R^{2s} is —H.
118. The oligonucleotide of any one of Embodiments 107-117, wherein an occurrence of R^{1s} is —H.
119. The oligonucleotide of any one of Embodiments 107-117, wherein each occurrence of R^{1s} is —H.

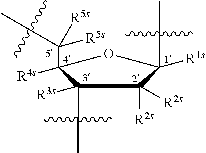

120. The oligonucleotide of Embodiment 107, wherein each is independently

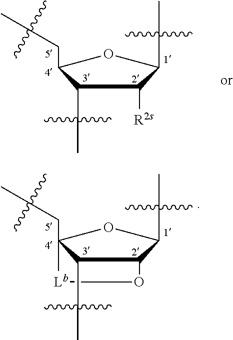

121. The oligonucleotide of any one of Embodiments 107-120, wherein an occurrence of R^{2s} is —H.
122. The oligonucleotide of any one of Embodiments 107-121, wherein an occurrence of R^{2s} is —F.
123. The oligonucleotide of any one of Embodiments 107-122, wherein an occurrence of R^{2s} is —OR, wherein R is optionally substituted C₁₋₆ alkyl.
124. The oligonucleotide of any one of Embodiments 107-123, wherein an occurrence of R^{2s} is —OMe.
125. The oligonucleotide of any one of Embodiments 107-124, wherein an occurrence of R^{2s} is —OCH₂CH₂OCH₃.
126. The oligonucleotide of any one of Embodiments 107-125, wherein an occurrence of R^{2s} is taken together with R^{4s}-OCH₂CH₂OCH₃.
127. The oligonucleotide of any one of Embodiments 107-126, wherein an occurrence of L^{b} is optionally substituted —CH₂—.
128. The oligonucleotide of any one of Embodiments 107-127, wherein each occurrence of L^{b} is independently optionally substituted —CH₂—.
129. The oligonucleotide of any one of Embodiments 107-128, wherein an occurrence of L^{b} is —CH₂—.
130. The oligonucleotide of any one of Embodiments 107-129, wherein each occurrence of L^{b} is —CH₂—.
131. The oligonucleotide of any one of the preceding Embodiments, wherein the oligonucleotide consists of or comprises a structure of 5′-a first region-a second region-a third region-3′, wherein each of the regions independently comprises 1-20 (e.g., 1, 2, 3, 4, 5, 6, 7, 8, 9, 10, 11, 12, 13, 14, 15, 16, 17, 18, 19, 20) or more nucleosides.
132. The oligonucleotide of any one of the preceding Embodiments, wherein the first region comprises 2, 3, 4, 5, 6, 7, 8, 9, 10, 11, 12, 13, 14, 15, 16, 17, 18, 19, or 20 or more nucleosides.
133. The oligonucleotide of any one of the preceding Embodiments, wherein the first region comprises 5 or more nucleosides.
134. The oligonucleotide of any one of the preceding Embodiments, wherein the second region comprises 2, 3, 4, 5, 6, 7, 8, 9, 10, 11, 12, 13, 14, 15, 16, 17, 18, 19, or 20 or more nucleosides.
135. The oligonucleotide of any one of the preceding Embodiments, wherein the second region comprises 4 or more nucleosides.
136. The oligonucleotide of any one of the preceding Embodiments, wherein the second region comprises 8 or more nucleosides.
137. The oligonucleotide of any one of the preceding Embodiments, wherein the second region comprises 10 or more nucleosides.
138. The oligonucleotide of any one of the preceding Embodiments, wherein the third region comprises 2, 3, 4, 5, 6, 7, 8, 9, 10, 11, 12, 13, 14, 15, 16, 17, 18, 19, or 20 or more nucleosides.
139. The oligonucleotide of any one of the preceding Embodiments, wherein the third region comprises 5 or more nucleosides.
140. The oligonucleotide of any one of the preceding Embodiments, wherein the pattern of sugar modifications of the first region differs from the pattern of sugar modifications of the third region.
141. The oligonucleotide of any one of Embodiments 1-140, wherein the pattern of sugar modifications of the first region is the same as the pattern of sugar modifications of the third region.
142. The oligonucleotide of any one of the preceding Embodiments, wherein the first region comprises one or more (e.g., 1-20, 1, 2, 3, 4, 5, 6, 7, 8, 9, 10, 11, 12, 13, 14, 15, 16, 17, 18, 19, or 20) 2′-F modified sugars.
143. The oligonucleotide of any one of the preceding Embodiments, wherein the first region comprises two or more (e.g., 2-20, 2, 3, 4, 5, 6, 7, 8, 9, 10, 11, 12, 13, 14, 15, 16, 17, 18, 19, or 20) consecutive 2′-F modified sugars.
144. The oligonucleotide of any one of the preceding Embodiments, wherein all 2′-F modified sugars in a first region are consecutive.
145. The oligonucleotide of any one of the preceding Embodiments, wherein at least 10%, 20%, 30%, 40%, 50%, 60%, 70%, 75%, 80%, 85%, 90%, or 95%, or 100% of all sugars in a first region comprises 2′-F.
146. The oligonucleotide of any one of the preceding Embodiments, wherein at least 50% of all sugars in a first region comprises 2′-F.
147. The oligonucleotide of any one of the preceding Embodiments, wherein one or more (e.g., 1, 2, 3, 4, 5, 6, 7, 8, 9, 10) sugars in the first region is independently

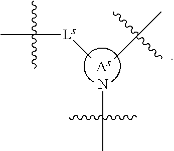

148. The oligonucleotide of any one of the preceding Embodiments, wherein one or more (e.g., 1, 2, 3, 4, 5, 6, 7, 8, 9, 10) sugars in the first region is independently an acyclic sugar.
149. The oligonucleotide of any one of Embodiments 1-146, wherein each sugar in a first region comprises 2′-F.
150. The oligonucleotide of any one of the preceding Embodiments, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) internucleotidic linkages in the first region are independently phosphorothioate internucleotidic linkages.
151. The oligonucleotide of Embodiment 150, wherein each phosphorothioate internucleotidic linkage in the first region is independently chirally controlled.
152. The oligonucleotide of Embodiment 150, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) phosphorothioate internucleotidic linkage in the first region are chirally controlled and are Sp.
153. The oligonucleotide of Embodiment 150, wherein each phosphorothioate internucleotidic linkage in the first region is chirally controlled and is Sp.
154. The oligonucleotide of any one of the preceding Embodiments, wherein the first region comprises one or more internucleotidic linkages each independently of the structure —O—P(O)[—N═C[N(R′)₂]₂]—O—.
155. The oligonucleotide of any one of the preceding Embodiments, wherein the first region comprises one or more n001 internucleotidic linkages.
156. The oligonucleotide of any one of Embodiments 154-155, wherein one or more of the internucleotidic linkages are independently chirally controlled.
157. The oligonucleotide of any one of Embodiments 154-155, wherein one or more of the internucleotidic linkages are independently chirally controlled and are Rp.
158. The oligonucleotide of any one of the preceding Embodiments, wherein the second region comprises one or more (e.g., 1-20, 1, 2, 3, 4, 5, 6, 7, 8, 9, 10, 11, 12, 13, 14, 15, 16, 17, 18, 19, or 20) 2′-F modified sugars.
159. The oligonucleotide of any one of the preceding Embodiments, wherein the second region comprises two or more (e.g., 2-20, 2, 3, 4, 5, 6, 7, 8, 9, 10, 11, 12, 13, 14, 15, 16, 17, 18, 19, or 20) consecutive 2′-F modified sugars.
160. The oligonucleotide of any one of the preceding Embodiments, wherein at least 10%, 20%, 30%, 40%, 50%, 60%, 70%, 75%, 80%, 85%, 90%, or 95%, or 100% of all sugars in a second region comprises 2′-F.
161. The oligonucleotide of any one of the preceding Embodiments, wherein at least 50% of all sugars in a second region comprises 2′-F.
162. The oligonucleotide of any one of the preceding Embodiments, wherein the second region comprises one or more (e.g., 1-20, 1, 2, 3, 4, 5, 6, 7, 8, 9, 10, 11, 12, 13, 14, 15, 16, 17, 18, 19, or 20) 2′-OR modified sugars, wherein R is optionally substituted C₁₋₆ aliphatic.
163. The oligonucleotide of any one of the preceding Embodiments, wherein the second region comprises two or more (e.g., 2-20, 2, 3, 4, 5, 6, 7, 8, 9, 10, 11, 12, 13, 14, 15, 16, 17, 18, 19, or 20) consecutive 2′-OR modified sugars, wherein R is optionally substituted C₁₋₆ aliphatic.
164. The oligonucleotide of any one of the preceding Embodiments, wherein at least 10%, 20%, 30%, 40%, 50%, 60%, 70%, 75%, 80%, 85%, 90%, or 95%, or 100% of all sugars in a second region comprises 2′-OR modified sugars, wherein R is optionally substituted C₁₋₆ aliphatic.
165. The oligonucleotide of any one of the preceding Embodiments, wherein at least 50% of all sugars in a second region comprises 2′-OR modified sugars, wherein R is optionally substituted C₁₋₆ aliphatic.
166. The oligonucleotide of any one of the preceding Embodiments, wherein all 2′-F modified sugars in a second region are consecutive.
167. The oligonucleotide of any one of the preceding Embodiments, wherein all 2′-OR modified sugars in a second region are consecutive.
168. The oligonucleotide of any one of Embodiments 131-165, wherein the second region comprise alternating 2′-F and 2′-OR modifications.
169. The oligonucleotide of any one of the preceding Embodiments, wherein one or more (e.g., 1, 2, 3, 4, 5, 6, 7, 8, 9, 10) sugars in the second region is independently

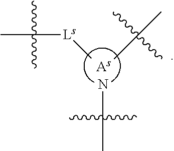

170. The oligonucleotide of any one of the preceding Embodiments, wherein one or more (e.g., 1, 2, 3, 4, 5, 6, 7, 8, 9, 10) sugars in the second region is independently an acyclic sugar.
171. The oligonucleotide of any one of Embodiments 131-168, wherein 2′-OR is 2′-OMe.
172. The oligonucleotide of any Embodiments 131-161, wherein each sugar in a second region comprises 2′-F.
173. The oligonucleotide of any Embodiments 134-157, wherein each sugar in a second region comprises two 2′-H.
174. The oligonucleotide of any Embodiments 134-157, wherein each sugar in a second region is independently a DNA sugar.
175. The oligonucleotide of any one of the preceding Embodiments, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) internucleotidic linkages in the second region are independently phosphorothioate internucleotidic linkages.
176. The oligonucleotide of Embodiment 175, wherein each phosphorothioate internucleotidic linkage in the second region is independently chirally controlled.
177. The oligonucleotide of Embodiment 175, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) phosphorothioate internucleotidic linkage in the second region are chirally controlled and are Sp.
178. The oligonucleotide of Embodiment 175, wherein each phosphorothioate internucleotidic linkage in the second region is chirally controlled and is Sp.
179. The oligonucleotide of any one of the preceding Embodiments, wherein the second region comprises one or more internucleotidic linkages each independently of the structure —O—P(O)[—N═C[N(R′)₂]₂]—O—.
180. The oligonucleotide of any one of the preceding Embodiments, wherein the second region comprises one or more n001 internucleotidic linkages.
181. The oligonucleotide of any one of Embodiments 179-180, wherein one or more of the internucleotidic linkages are independently chirally controlled.
182. The oligonucleotide of any one of Embodiments 179-180, wherein one or more of the internucleotidic linkages are independently chirally controlled and are Rp.
183. The oligonucleotide of any one of the preceding Embodiments, wherein the third region comprises one or more (e.g., 1-20, 1, 2, 3, 4, 5, 6, 7, 8, 9, 10, 11, 12, 13, 14, 15, 16, 17, 18, 19, or 20) 2′-F modified sugars.
184. The oligonucleotide of any one of the preceding Embodiments, wherein the third region comprises two or more (e.g., 2-20, 2, 3, 4, 5, 6, 7, 8, 9, 10, 11, 12, 13, 14, 15, 16, 17, 18, 19, or 20) consecutive 2′-F modified sugars.
185. The oligonucleotide of any one of the preceding Embodiments, wherein all 2′-F modified sugars in a third region are consecutive.
186. The oligonucleotide of any one of the preceding Embodiments, wherein at least 10%, 20%, 30%, 40%, 50%, 60%, 70%, 75%, 80%, 85%, 90%, or 95%, or 100% of all sugars in a third region comprises 2′-F.
187. The oligonucleotide of any one of the preceding Embodiments, wherein at least 10%, 20%, 30%, 40%, 50%, 60%, 70%, 75%, 80%, 85%, 90%, or 95%, or 100% of all sugars in a third region comprises 2′-OR modified sugars, wherein R is optionally substituted C₁₋₆ aliphatic.
188. The oligonucleotide of any one of the preceding Embodiments, wherein at least 50% of all sugars in a third region comprises 2′-F.
189. The oligonucleotide of any one of the preceding Embodiments, wherein one or more (e.g., 1, 2, 3, 4, 5, 6, 7, 8, 9, 10) sugars in the first region is independently

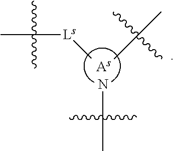

190. The oligonucleotide of any one of the preceding Embodiments, wherein one or more (e.g., 1, 2, 3, 4, 5, 6, 7, 8, 9, 10) sugars in the first region is independently an acyclic sugar. 191. The oligonucleotide of any one of Embodiments 1-190, wherein each sugar in a third region comprises 2′-F.
192. The oligonucleotide of any one of the preceding Embodiments, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) internucleotidic linkages in the third region are independently phosphorothioate internucleotidic linkages.
193. The oligonucleotide of Embodiment 192, wherein each phosphorothioate internucleotidic linkage in the third region is independently chirally controlled.
194. The oligonucleotide of Embodiment 192, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) phosphorothioate internucleotidic linkage in the third region are chirally controlled and are Sp.
195. The oligonucleotide of Embodiment 192, wherein each phosphorothioate internucleotidic linkage in the third region is chirally controlled and is Sp.
196. The oligonucleotide of any one of the preceding Embodiments, wherein the third region comprises one or more internucleotidic linkages each independently of the structure —O—P(O)[—N═C[N(R′)₂]₂]—O—.
197. The oligonucleotide of any one of the preceding Embodiments, wherein the third region comprises one or more n001 internucleotidic linkages.
198. The oligonucleotide of any one of Embodiments 196-197, wherein one or more of the internucleotidic linkages are independently chirally controlled.
199. The oligonucleotide of any one of Embodiments 196-197, wherein one or more of the internucleotidic linkages are independently chirally controlled and are Rp.
200. The oligonucleotide of any one of Embodiments 1-141, wherein the first region comprises one or more sugars comprising a 2′-modification.
201. The oligonucleotide of Embodiment 200, wherein a sugar comprises a 2′-modificatoin which 2′-OR, wherein R is optionally substituted C₁₋₆ alkyl.
202. The oligonucleotide of Embodiment 200 or 201, wherein a sugar comprises 2′-OMe.
203. The oligonucleotide of any one of Embodiments 200-202, wherein a sugar comprises 2′-MOE.
204. The oligonucleotide of any one of Embodiments 1-141, wherein the first region comprises one or more sugars having the structure of

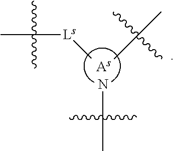

205. The oligonucleotide of any one of Embodiments 1-141, wherein the first region comprises two or more consecutive sugars each independently having the structure of

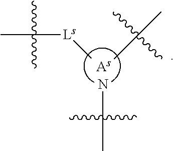

206. The oligonucleotide of any one of Embodiments 200-205, wherein each sugar in the first region is independently a sugar comprising a 2′-modification or a sugar having the structure of

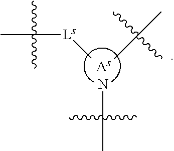

207. The oligonucleotide of any one of Embodiments 204-206, wherein the N is bonded to the P of an internucleotidic linkage having the structure of —P(═W)(—X-L^{L}-R^{L})—Z—, and a nucleobase is bonded to Ring A^{s}.
208. The oligonucleotide of any one of Embodiments 200-207, wherein a first region comprises one or more natural phosphate linkages.
209. The oligonucleotide of any one of Embodiments 200-208, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) internucleotidic linkages in the first region are independently phosphorothioate internucleotidic linkages.
210. The oligonucleotide of Embodiment 209, wherein each phosphorothioate internucleotidic linkage in the third region is independently chirally controlled.
211. The oligonucleotide of Embodiment 209, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) phosphorothioate internucleotidic linkage in the third region are chirally controlled and are Sp.
212. The oligonucleotide of Embodiment 209, wherein each phosphorothioate internucleotidic linkage in the third region is chirally controlled and is Sp.
213. The oligonucleotide of any one of Embodiments 200-212, wherein the third region comprises one or more internucleotidic linkages each independently of the structure —O—P(O)[—N═C[N(R′)₂]₂]—O—.
214. The oligonucleotide of Embodiment 213, wherein the third region comprises one or more n001 internucleotidic linkages.
215. The oligonucleotide of any one of Embodiments 213-214, wherein one or more of the internucleotidic linkages are independently chirally controlled.
216. The oligonucleotide of any one of Embodiments 213-214, wherein one or more of the internucleotidic linkages are independently chirally controlled and are Rp.
217. The oligonucleotide of any one of Embodiments 1-141 and 200-216, wherein the second region comprises no sugar comprising a 2′-OR.
218. The oligonucleotide of any one of Embodiments 1-141 and 200-216, wherein each sugar in the second region is independently a sugar comprising two 2′-H, or a sugar having the structure of

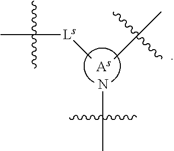

219. The oligonucleotide of any one of Embodiments 1-141 and 200-216, wherein each sugar in the second region is independently a sugar comprising two 2′-H.
220. The oligonucleotide of any one of Embodiments 1-141 and 200-216, wherein one or more (e.g., 1, 2, 3, 4, 5, 6, 7, 8, 9, 10 or more) sugars in the second region are independently a sugar having the structure of

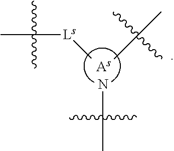

221. The oligonucleotide of any one of Embodiments 218-220, wherein the N is bonded to the P of an internucleotidic linkage having the structure of P(═W)(—X-L^{L}-R^{L})—Z—, and a nucleobase is bonded to Ring A^{s}.
222. The oligonucleotide of any one of Embodiments 1-141 and 200-221, wherein the pattern of backbone chiral centers (linkage phosphorus) of the second region is or comprises: [(Op/Rp/Mp)n(Sp)m]y, or (Np)t[(Op/Rp/Mp)n(Sp)m]y,
   wherein:

t is 1-50;

n is 1-10;

m is 1-50;

y is 1-10;

Np is either Rp or Sp;

Sp indicates the S configuration of a chiral linkage phosphorus of a chiral modified internucleotidic linkage;

Mp indicates the configuration of a chiral linkage phosphorus of a chiral modified internucleotidic linkage bonded to the N of a sugar having the structure of

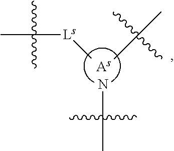

wherein Mp is Sp or Rp, or wherein the chiral modified internucleotidic linkage is not chirally controlled, is Xp;

Op indicates an achiral linkage phosphorus of a natural phosphate linkage; and

Rp indicates the S configuration of a chiral linkage phosphorus of a chiral modified internucleotidic linkage; and

y is 1-10.
223. The oligonucleotide of any one of the preceding Embodiments, wherein the pattern of backbone chiral centers of the second region is or comprises (Np)t[(Op/Rp/Mp)n(Sp)m]y.
224. The oligonucleotide of any one of the preceding Embodiments, wherein the pattern of backbone chiral centers of the second region is (Np)t[(Op/Rp/Mp)n(Sp)m]y.
225. The oligonucleotide of any one of the preceding Embodiments, wherein each Np is Sp.
226. The oligonucleotide of any one of the preceding Embodiments, wherein at least one (Op/Rp/Mp) is Rp.
227. The oligonucleotide of any one of the preceding Embodiments, wherein at least one (Op/Rp/Mp) is Op.
228. The oligonucleotide of any one of the preceding Embodiments, wherein at least one (Op/Rp/Mp) is Mp.
229. The oligonucleotide of any one of the preceding Embodiments, wherein the pattern of backbone chiral centers of the second region is or comprises [(Mp)n(Sp)m]y.
230. The oligonucleotide of any one of the preceding Embodiments, wherein the pattern is or comprises (Np)t[(Rp)n(Sp)m]y.
231. The oligonucleotide of any one of the preceding Embodiments, wherein the pattern comprises at least one Rp.
232. The oligonucleotide of any one of the preceding Embodiments, wherein at least one n is 1.
233. The oligonucleotide of any one of the preceding Embodiments, wherein each n is 1.
234. The oligonucleotide of any one of the preceding Embodiments, wherein y is 1.
235. The oligonucleotide of any one of Embodiments 1-233, wherein y is 2.
236. The oligonucleotide of any one of the preceding Embodiments, wherein t is 2 or more.
237. The oligonucleotide of any one of the preceding Embodiments, wherein t is 2-20
238. The oligonucleotide of any one of the preceding Embodiments, wherein t is 3 or more.
239. The oligonucleotide of any one of the preceding Embodiments, wherein t is 3-20.
240. The oligonucleotide of any one of the preceding Embodiments, wherein at least one m is 2-20.
241. The oligonucleotide of any one of the preceding Embodiments, wherein at least one m is 2.
242. The oligonucleotide of any one of the preceding Embodiments, wherein at least one m is 3, 4, 5, 6, 7, 8, 9, or 10.
243. The oligonucleotide of any one of the preceding Embodiments, wherein each m is independently 2-20.
244. The oligonucleotide of any one of the preceding Embodiments, wherein the first occurrence of [(Op/Rp/Mp)n(Sp)m]y in a second region from the 5′ is RpSpSp.
245. The oligonucleotide of any one of the preceding Embodiments, wherein the first occurrence of [(Op/Rp/Mp)n(Sp)m]y in a second region from the 5′ is RpSpSpSp.
246. The oligonucleotide of any one of the preceding Embodiments, wherein the first occurrence of [(Op/Rp/Mp)n(Sp)m]y in a second region from the 5′ is RpSpSpSpSp.
247. The oligonucleotide of any one of the preceding Embodiments, wherein the first occurrence of [(Op/Rp/Mp)n(Sp)m]y in a second region from the 3′ is RpSpSp.
248. The oligonucleotide of any one of the preceding Embodiments, wherein the first occurrence of [(Op/Rp/Mp)n(Sp)m]y in a second region from the 3′ is RpSpSpSp.
249. The oligonucleotide of any one of the preceding Embodiments, wherein the first occurrence of [(Op/Rp/Mp)n(Sp)m]y in a second region from the 3′ is RpSpSpSpSp.
250. The oligonucleotide of any one of the preceding Embodiments, wherein the last Sp of a [(Op/Rp)(Sp)m]y motif in a second region represents the linkage phosphorus configuration of internucleotidic linkage bonded to a sugar of a second region and a sugar of the third region.
251. The oligonucleotide of any one of the preceding Embodiments, wherein the pattern of backbone chiral centers of a core comprises or is [(Rp(Sp)m]y, (Np)t[Rp(Sp)m]y, or (Sp)t[Rp(Sp)m]y.
252. The oligonucleotide of any one of the preceding Embodiments, wherein there are about or at least about 1-20, e.g., about or at least about 1, 2, 3, 4, 5, 6, 7, 8, 9, 10, 11, 12, 13, 14, 15, 16, 17, 18, 19, or 20 internucleotidic linkages, each of which is independently bonded to one or more core sugars, to the 5′ side of a core internucleotidic linkage whose configuration is the Rp of [(Rp(Sp)m]y, (Np)t[Rp(Sp)m]y, or (Sp)t[Rp(Sp)m]y.
253. The oligonucleotide of any one of the preceding Embodiments, wherein there are about 3-10 internucleotidic linkages, each of which is independently bonded to one or more core sugars, to the 5′ side of a core internucleotidic linkage whose configuration is the Rp of [(Rp(Sp)m]y, (Np)t[Rp(Sp)m]y, or (Sp)t[Rp(Sp)m]y.
254. The oligonucleotide of any one of the preceding Embodiments, wherein the internucleotidic linkage whose configuration is the Rp of [(Rp(Sp)m]y, (Np)t[Rp(Sp)m]y, or (Sp)t[Rp(Sp)m]y is the 5^{th}, 6^{th}, 7^{th}, 8^{th}, 9^{th}, 10^{th}, 11^{th} or 12^{th} internucleotidic linkage that is bonded to at least one core sugar.
255. The oligonucleotide of any one of the preceding Embodiments, wherein a sugar comprising nitrogen is at position +1, +2, +3, +4, +5, +6, +7, +8, −1, −2, −3, −4, −5, −6, −7, or −8 relative to the Rp internucleotidic linkage (5′- . . . N₊₄ N₊₃ N₊₂ N₊₁ N₋₁ N₋₂ N₋₃ N₋₄. . . -3′, wherein Rp is the configuration of the internucleotidic linkage connecting N₊₁ and N₋₁).
256. The oligonucleotide of Embodiment 255, wherein the position is +7.
257. The oligonucleotide of Embodiment 255, wherein the position is +6.
258. The oligonucleotide of Embodiment 255, wherein the position is +4.
259. The oligonucleotide of Embodiment 255, wherein the position is +3.
260. The oligonucleotide of Embodiment 255, wherein the position is −2.
261. The oligonucleotide of Embodiment 255, wherein the position is −3.
262. The oligonucleotide of any one of Embodiments 255-261, wherein the sugar comprising nitrogen has the structure of

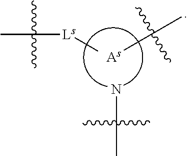

263. The oligonucleotide of any one of Embodiments 102-262, wherein each Rp in a pattern of backbone chiral centers is independently of a phosphorothioate internucleotidic linkage.
264. The oligonucleotide of any one of Embodiments 102-263, wherein each Sp in a pattern of backbone chiral centers is independently of a phosphorothioate internucleotidic linkage.
265. The oligonucleotide of any one of Embodiments 1-141 and 200-264, wherein the third region comprises one or more sugars comprising a 2′-modification.
266. The oligonucleotide of Embodiment 265, wherein a sugar comprises a 2′-modificatoin which 2′-OR, wherein R is optionally substituted C₁₋₆ alkyl.
267. The oligonucleotide of Embodiment 265 or 266, wherein a sugar comprises 2′-OMe.
268. The oligonucleotide of any one of Embodiments 265-267, wherein a sugar comprises 2′-MOE.
269. The oligonucleotide of any one of Embodiments 1-141 and 200-268, wherein the third region comprises one or more sugars having the structure of

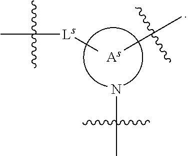

270. The oligonucleotide of any one of Embodiments 1-141 and 200-268, wherein the third region comprises two or more consecutive sugars each independently having the structure of

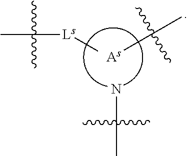

271. The oligonucleotide of any one of Embodiments 1-141 and 200-270, wherein each sugar in the third region is independently a sugar comprising a 2′-modification or a sugar having the structure of

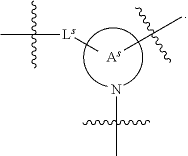

272. The oligonucleotide of any one of Embodiments 269-271, wherein the N is bonded to the P of an internucleotidic linkage having the structure of —P(═W)(—X-L^{L}-R^{L})—Z—, and a nucleobase is bonded to Ring A^{s}.
273. The oligonucleotide of any one of Embodiments 200-272, wherein a third region comprises one or more natural phosphate linkages.
274. The oligonucleotide of any one of Embodiments 200-273, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) internucleotidic linkages in the third region are independently phosphorothioate internucleotidic linkages.
275. The oligonucleotide of Embodiment 274, wherein each phosphorothioate internucleotidic linkage in the third region is independently chirally controlled.
276. The oligonucleotide of Embodiment 274, wherein one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) phosphorothioate internucleotidic linkage in the third region are chirally controlled and are Sp.
277. The oligonucleotide of Embodiment 274, wherein each phosphorothioate internucleotidic linkage in the third region is chirally controlled and is Sp.
278. The oligonucleotide of any one of Embodiments 200-277, wherein the third region comprises one or more internucleotidic linkages each independently of the structure —O—P(O)[ N═C[N(R′)₂]₂]—O—.
279. The oligonucleotide of Embodiment 278, wherein the third region comprises one or more n001 internucleotidic linkages.
280. The oligonucleotide of any one of Embodiments 278-279, wherein one or more of the internucleotidic linkages are independently chirally controlled.
281. The oligonucleotide of any one of Embodiments 278-279, wherein one or more of the internucleotidic linkages are independently chirally controlled and are Rp.
282. The oligonucleotide of any one of Embodiments 131-281, wherein Z is —O—.
283. The oligonucleotide of any one of Embodiments 131-282, wherein W is —O—.
284. The oligonucleotide of any one of Embodiments 131-283, wherein in X is —S—.
285. The oligonucleotide of any one of Embodiments 131-283, wherein in X is —O—.
286. The oligonucleotide of any one of Embodiments 284-285, wherein R^{L} is H.
287. The oligonucleotide of any one of Embodiments 131-283, wherein in —X—R^{L} is —N(R′)-L^{L}-R′.
288. The oligonucleotide of any one of Embodiments 131-283, wherein in —X—R^{L} is N═C(-L^{L}-R′)₂.
289. The oligonucleotide of any one of Embodiments 131-283, wherein in —X—R^{L} is —N═C(-L^{L}-R′)[N(R′)₂].
290. The oligonucleotide of any one of Embodiments 131-283, wherein in —X—R^{L} is —N═C[N(R′)₂]₂.
291. The oligonucleotide of any one of Embodiments 131-290, wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms.
292. The oligonucleotide of Embodiment 291, wherein one R′ on one N and one R′ or the other N are taken together with their intervening atoms to form an optionally substituted, 5-membered, monocyclic, bicyclic or polycyclic ring having, in addition to the intervening atoms, 0 heteroatoms.
293. The oligonucleotide of any one of Embodiments 291-292, wherein the formed ring is saturated.
294. The oligonucleotide of any one of Embodiments 291-293, wherein R^{L} is

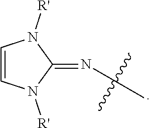

295. The oligonucleotide of any one of Embodiments 291-296, wherein R^{L} is

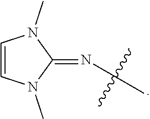 [SMILES: *N=c1n(C)ccn1C]

296. The oligonucleotide of any one of Embodiments 291-293, wherein R^{L} is

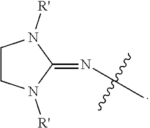

297. The oligonucleotide of any one of Embodiments 291-296, wherein R^{L} is

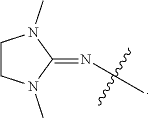 [SMILES: *N=C1N(C)CCN1C]

298. The oligonucleotide of any one of 131-297, wherein

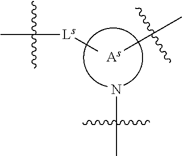

is optionally substituted

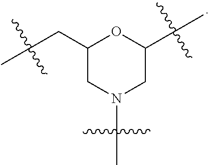 [SMILES: *CC1CN(*)CC(*)O1]

299. The oligonucleotide of Embodiment 298, wherein

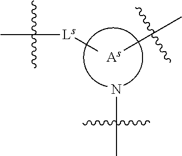

is optionally substituted

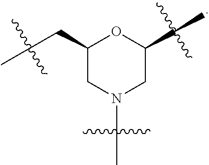 [SMILES: *C[C@@H]1CN(*)C[C@H](*)O1]

300. The oligonucleotide of Embodiment 298, wherein

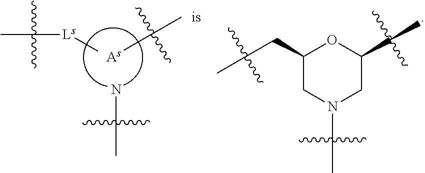

301. The oligonucleotide of any one of the preceding Embodiments, wherein the oligonucleotide comprises one or more non-negatively charged internucleotidic linkages having the structure of formula I, I-a-1, I-a-2, I-b, I-c, I-d, I-e, I-n-1, I-n-2, I-n-3, I-n-4, II, II-a-1, II-a-2, II-b-1, II-b-2, II-c-1, II-c-2, II-d-1, or II-d-2.
302. The oligonucleotide of any one of the preceding Embodiments, wherein the oligonucleotide comprises one or more neutral internucleotidic linkages having the structure of formula I, I-a-1, I-a-2, I-b, I-c, I-d, I-e, I-n-1, I-n-2, I-n-3, I-n-4, II, II-a-1, II-a-2, II-b-1, II-b-2, II-c-1, II-c-2, II-d-1, or II-d-2.
303. The oligonucleotide of any one of the preceding Embodiments, wherein each nucleobase is independently optionally substituted A, T, C, G or U, or an optionally substituted tautomer of A, T, C, G or U.
304. The oligonucleotide of any one of the preceding Embodiments, wherein the oligonucleotide chain is conjugated with an additional moiety which is or comprises a lipid moiety, a carbohydrate moiety, and/or a targeting moiety.
305. The oligonucleotide of Embodiment 304, wherein the additional moiety is or comprises

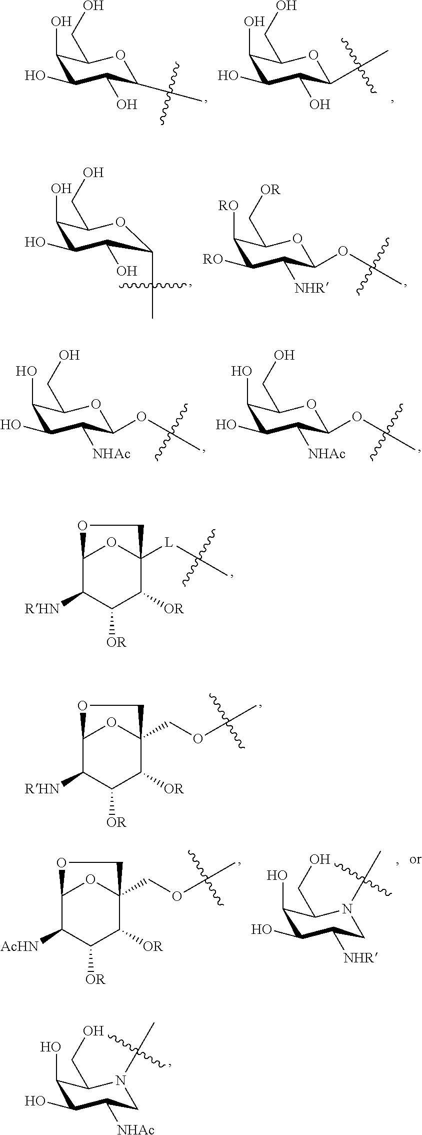

306. A compound comprising

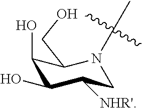

307. An oligonucleotide comprising

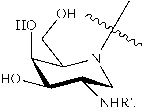

308. The oligonucleotide of Embodiment 304 or 307, wherein the oligonucleotide comprises two or more

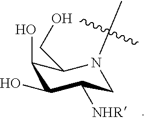

309. The oligonucleotide of Embodiment 304 or 307, wherein the oligonucleotide comprises three or more

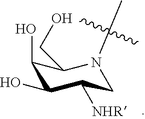

310. The oligonucleotide of any one of Embodiments 305-309, wherein R′ is —Ac.
311. The oligonucleotide of Embodiment 304, wherein the additional moiety is or comprises

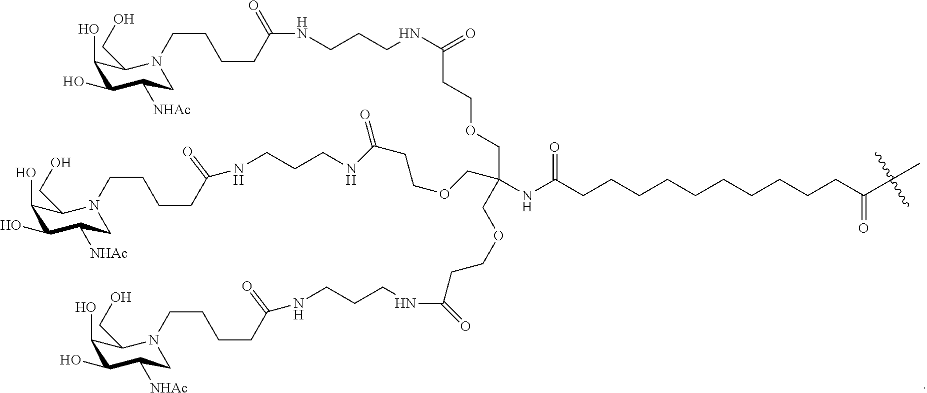

312. The oligonucleotide of Embodiment 304, wherein the additional moiety is or comprises

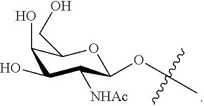

313. The oligonucleotide of Embodiment 304, wherein the additional moiety is or comprises

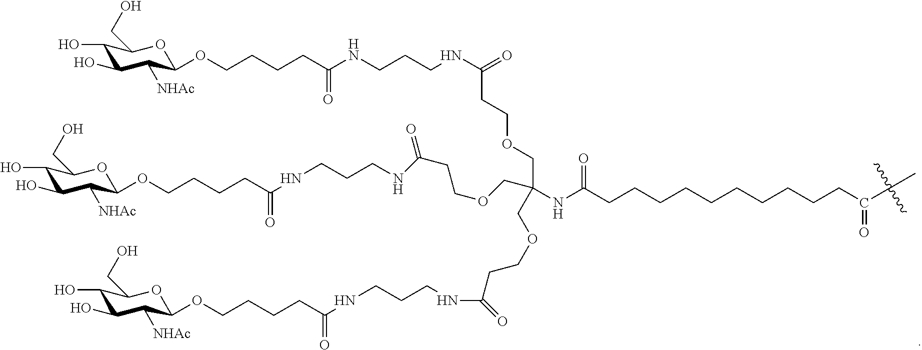

314. The oligonucleotide of any one of the preceding Embodiments, wherein the oligonucleotide is in a form of a pharmaceutically acceptable salt.
315. The oligonucleotide of any one of the preceding Embodiments, wherein the oligonucleotide is a sodium salt form.
316. The oligonucleotide of any one of the preceding Embodiments, wherein each chirally controlled phosphorothioate internucleotidic linkage of the oligonucleotide independently has a diastereomeric purity of at least 85%, 90%, 95%, 96%, 97%, 98%, or 99%.
317. The oligonucleotide of any one of the preceding Embodiments, wherein each chirally controlled chiral internucleotidic linkage of the oligonucleotide independently has a diastereomeric purity of at least 55%, 60%, 70%, 80%, 85%, 90%, 95%, 96%, 97%, 98%, or 99%.
318. The oligonucleotide of any one of the preceding Embodiments, wherein the oligonucleotide has a diastereomeric purity of at least (DS)^{nc}, wherein DS is 55%-100% (e.g., about or at least about 55%, 60%, 70%, 80%, 85%, 90%, 95%, 96%, 97%, 98%, or 99%), and nc is the number of chirally controlled internucleotidic linkages.
319. The oligonucleotide of Embodiment 318, wherein DS is about 80%.
320. The oligonucleotide of Embodiment 318, wherein DS is about 85%.
321. The oligonucleotide of Embodiment 318, wherein DS is about 90%.
322. The oligonucleotide of Embodiment 318, wherein DS is about 95% or more.
323. The oligonucleotide of any one of the preceding Embodiments, wherein the oligonucleotide has a diastereomeric purity of about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%.
324. The oligonucleotide of any one of the preceding Embodiments, wherein the oligonucleotide has a diastereomeric purity of at least 50%, 60%, 70%, 80%, 85%, 90%, 95%, 96%, 97%, 98%, or 99%.
325. A chirally controlled oligonucleotide composition comprising a plurality of oligonucleotides, wherein the oligonucleotides share:

1) a common base sequence,

2) a common pattern of backbone linkages, and

3) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),

wherein about 1-100% of all oligonucleotides within the composition that share the common base sequence and common pattern of backbone linkages are the oligonucleotides of the plurality,

each oligonucleotide of the plurality is independently an oligonucleotide of any one of Embodiments 1-315.
326. A chirally controlled oligonucleotide composition comprising a plurality of oligonucleotides, wherein the oligonucleotides share:

1) a common constitution, and

2) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),

wherein the composition is enriched, relative to a substantially racemic preparation of oligonucleotides sharing the common constitution, for oligonucleotides of the plurality, and

each oligonucleotide of the plurality is independently an oligonucleotide of any one of Embodiments 1-315.
327. The composition of Embodiment 326, wherein 5%, 10%, 15%, 20%, 25%, 30%, 35%, 40%, 45%, 50%, 60%, 70%, 75%, 80%, 85%, 90%, 95%, 96%, 97%, 98%, 99% or more of all oligonucleotides within the composition that share the common constitution are the oligonucleotides of the plurality.
328. A chirally controlled oligonucleotide composition comprising a plurality of oligonucleotides, wherein the oligonucleotides share:

1) a common constitution, and

2) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),

wherein about 1-100% of all oligonucleotides within the composition that share the common constitution are the oligonucleotides of the plurality, and

each oligonucleotide of the plurality is independently an oligonucleotide of any one of Embodiments 1-315.
329. The composition of any one of the preceding Embodiments, wherein oligonucleotides of the plurality share the same linkage phosphorus stereochemistry at 5 or more chiral internucleotidic linkages.
330. The composition of any one of the preceding Embodiments, wherein oligonucleotides of the plurality share the same linkage phosphorus stereochemistry independently at each phosphorothioate internucleotidic linkage.
331. The composition of any one of the preceding Embodiments, wherein oligonucleotides of the plurality share the same linkage phosphorus stereochemistry independently at each non-negatively charged internucleotidic linkage.
332. The composition of any one of the preceding Embodiments, wherein oligonucleotides of the plurality share the same Rp linkage phosphorus stereochemistry independently at each non-negatively charged internucleotidic linkage.
333. The composition of any one of the preceding Embodiments, wherein about 1-100% of all oligonucleotides within the composition that share the common base sequence are oligonucleotides of the plurality.
334. An oligonucleotide composition comprising a plurality of oligonucleotides, wherein:

each oligonucleotide of the plurality is independently a particular oligonucleotide or a salt thereof,

about 1-100% of all oligonucleotides within the composition that share the same constitution as the particular oligonucleotide or a salt thereof are oligonucleotides of the plurality, and

the particular oligonucleotide is an oligonucleotide of any one of Embodiments 1-315.

335. An oligonucleotide composition comprising a plurality of oligonucleotides, wherein:

each oligonucleotide of the plurality is independently a particular oligonucleotide or a salt thereof,

about 1-100% of all oligonucleotides within the composition that share the same base sequence as the particular oligonucleotide or a salt thereof are oligonucleotides of the plurality, and

the particular oligonucleotide is an oligonucleotide of any one of Embodiments 1-315.
336. The composition of any one of the preceding Embodiments, wherein the percentage is about or more than about (DS)^{nc}, wherein DS is 55%-100% (e.g., about or at least about 55%, 60%, 70%, 80%, 85%, 90%, 95%, 96%, 97%, 98%, or 99%), and nc is the number of chirally controlled internucleotidic linkages among oligonucleotides of the plurality.
337. The oligonucleotide of Embodiment 336, wherein DS is about 80%.
338. The oligonucleotide of Embodiment 336, wherein DS is about 85%.
339. The oligonucleotide of Embodiment 336, wherein DS is about 90%.
340. The oligonucleotide of Embodiment 336, wherein DS is about 95% or more.
341. The composition of any one of the preceding Embodiments, wherein the percentage is 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%.
342. The composition of any one of the preceding Embodiments, wherein the percentage is 5%, 10%, 15%, 20%, 25%, 30%, 35%, 40%, 45%, 50%, 60%, 70%, 75%, 80%, 85%, 90%, 95%, 96%, 97%, 98%, 99% or more.
343. The composition of any one of the preceding Embodiments, wherein the percentage is 50% or more.
344. The composition of any one of the preceding Embodiments, wherein oligonucleotides of the plurality are identical.
345. The composition of any one of Embodiments 218-224, wherein oligonucleotides of the plurality are each independently in a pharmaceutically acceptable salt form.
346. The composition of any one of Embodiments 218-225, wherein oligonucleotides of the plurality are each a sodium salt.
347. The composition of any one of Embodiments 218-225, wherein oligonucleotides of the plurality are in two or more pharmaceutically acceptable salt forms.
348. A pharmaceutical composition comprising or delivering an oligonucleotide or a composition of any one of the preceding Embodiments and a pharmaceutically acceptable carrier.
349. The composition of Embodiment 348, wherein the oligonucleotide is a pharmaceutically acceptable salt form.
350. The composition of Embodiment 349, wherein the oligonucleotide is a sodium salt form.
351. A method for modulating expression, level and/or activity of a target nucleic acid and/or a product thereof, comprising contacting the target nucleic acid with an oligonucleotide or composition of any one of the preceding Embodiments, wherein the base sequence of the oligonucleotide, or the common base sequence of oligonucleotides of a plurality in a composition, is complementary to that of the target nucleic acid.
352. A method, comprising administering to a system expressing a target nucleic acid an oligonucleotide or composition of any one of the preceding Embodiments, wherein the base sequence of the oligonucleotide, or the common base sequence of oligonucleotides of a plurality in a composition, is complementary to that of the target nucleic acid.
353. The method of Embodiment 352, wherein the system is a cell.
354. The method of Embodiment 352, wherein the system is a tissue.
355. The method of Embodiment 352, wherein the system is an organ.
356. The method of Embodiment 352, wherein the system is a subject.
357. The method of any one of Embodiments 351-356, wherein expression, level and/or activity of a target nucleic acid and/or a product thereof is reduced.
358. The method of any one of Embodiments 357, wherein a product is mRNA.
359. The method of any one of Embodiments 357, wherein a product is a protein.
360. The method of any one of Embodiments 351-356, wherein expression, level and/or activity of a product is increased, wherein the product is mRNA or a protein encoded thereby.
361. The method of any one of Embodiment 360, wherein the mRNA is a product of splicing modulation.
362. The method of any one of Embodiment 360, wherein the mRNA is a product of exon skipping.
363. The method of Embodiment 351, wherein the system is a human.
364. A compound of formula AC-I or a salt thereof.
365. A compound of formula AC-I-a or a salt thereof.
366. A compound of formula AC-I-b or a salt thereof.
367. A compound of formula AC-I-c or a salt thereof.
368. A compound of formula AC-I-d or a salt thereof.
369. A compound of formula AC-I-e or a salt thereof.
370. A compound selected from

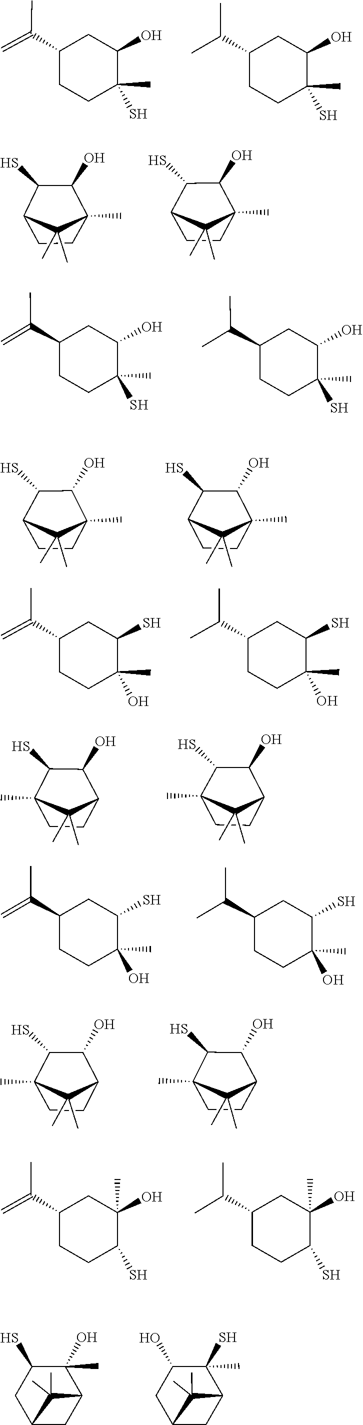

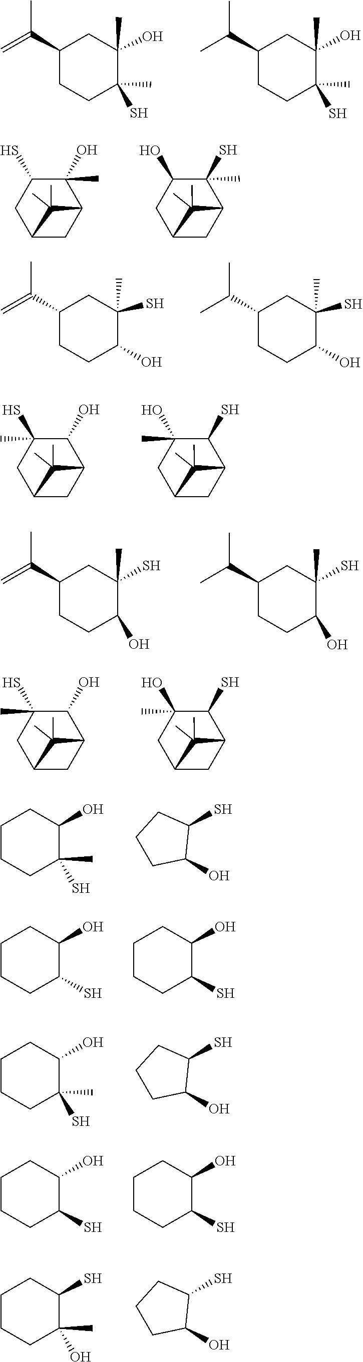

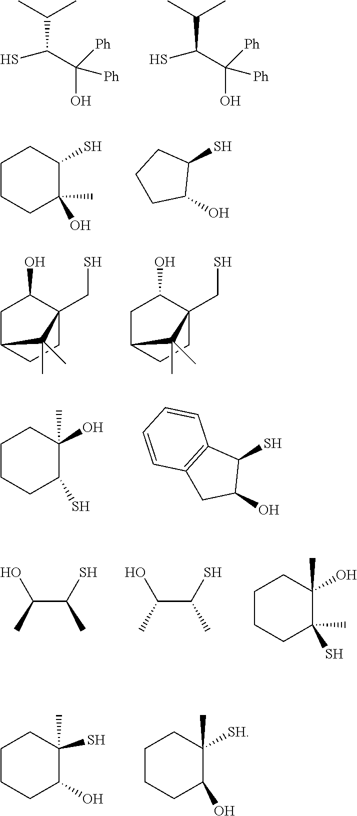

371. A compound having the structure of LG-I:

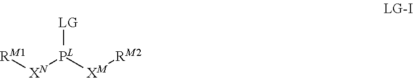

or a salt thereof, wherein:

LG is a leaving group;

each of X^{M} and X^{N} is independently -L-O—, -L-S— or -L-NR^{MN}—;

P^{L} is P, P(═W), P->B(-L^{L}-R^{L})₃, or P^{N};

W is O, N(-L^{L}-R^{L}), S or Se;

P^{N} is P═N—C(-L^{L}-R′)(=L^{N}-R′) or P═N-L^{L}-R^{L};

L^{N} is ═N-L^{L1}-, ═CH-L^{L1}- wherein CH is optionally substituted, or ═N⁺(R′)(Q⁻)-L^{L1}-;

each L″ is independently L;

Q⁻ is an anion;

each of R^{M1}, R^{M2} and R^{MN} is independently -L^{M}—R^{M};

each R^{L} is independently -L^{L}-R′ or —N═C(-L^{L}-R′)₂;

each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

each of L^{L} and L^{M} is independently L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)——P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)—, —OP(SR′)—, —OP(NR′)—, —OP(R′)—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
372. The compound of Embodiment 367, wherein X^{M} is —5—.
373. The compound of Embodiment 367, wherein X^{M} is —NR^{MN}—.
374. The compound of any one of Embodiments 367-373, wherein R^{M1} and R^{Ma}are taken together with their intervening atoms to form an optionally substituted, 3-30 membered, monocyclic, bicyclic or polycyclic ring having, in addition to the intervening atoms, 0-10 heteroatoms.
375. The compound of Embodiment 374, wherein the formed ring is 5-membered.
376. The compound of Embodiment 374, wherein the formed ring is 6-membered.
377. The compound of any one of Embodiments 374-376, wherein the formed ring is substituted.
378. The compound of any one of Embodiments 374-377, wherein the formed ring is monocyclic.
379. The compound of any one of Embodiments 374-377, wherein the formed ring is bicyclic.
380. The compound of any one of Embodiments 374-379, wherein the formed ring is saturated.
381. The compound of any one of Embodiments 374-380, wherein the formed ring has no heteroatoms in addition to the intervening atoms.
382. The compound of Embodiment 373, wherein R^{M1}, R^{M2} and R^{AIN} are taken together with their intervening atoms to form an optionally substituted 3-30 membered, monocyclic, bicyclic or polycyclic ring having 0-10 heteroatoms.
383. The compound of Embodiment 382, wherein the formed ring is an optionally substituted 8-10 membered bicyclic ring.
384. The compound of any one of Embodiments 382-383, wherein the formed ring is an optionally substituted [5,5]-fused bicyclic ring.
385. The compound of any one of Embodiments 382-384, wherein the formed ring comprise no heteroatoms in addition to the intervening atoms.
386. The compound of any one of Embodiments 382-385, wherein the formed ring is saturated.
387. The compound of any one of Embodiments 367-386, wherein LG is —Cl.
388. The compound of any one of Embodiments 367-386, wherein LG is —N(R′)_{2.}
389. The compound of any one of Embodiments 367-386, wherein LG is —N(iPr)_{2.}
390. A compound having the structure of LG-II:

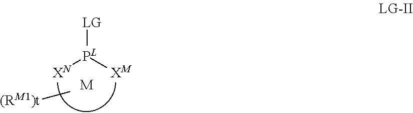

or a salt thereof, wherein:

LG is a leaving group; each of X^{M} and X^{N} is independently -L-O—, -L-S— or -L-NR^{1vIN}—; P^{L} is P, P(═W), P->B(-L^{L}-R^{L})₃, or P^{N}; W is O, N(-L^{L}-R^{L}), S or Se; each R^{L} is independently -L^{L}-R′ or —N═C(-L^{L}-R′)₂; P^{N} is P═N—C(-L^{L}-R′)(=L^{N}—R′) or P═N-L^{L}-R^{L}; L^{N} is ═N-L″—,=CH-L″—wherein CH is optionally substituted, or=1\1⁺(W)(Q⁻)-L″—; each L″ is independently L; Q⁻ is an anion; each of R^{M1} and R^{MN} is independently -LM—RM;

each R^{L} is independently -L^{L}-R′ or —N═C(-L^{L}-R′)₂; each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

t is 0-10; each of L^{L} and L^{M} is independently L; Ring M is an optionally substituted 3-30 membered, monocyclic, bicyclic or polycyclic ring having 0-10 heteroatoms;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C E C a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R; each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or two R groups are optionally and independently taken together to form a covalent bond, or: two or more R groups on the same atom are optionally and independently taken together with the atom to form an optionally substituted, 3-30 membered, monocyclic, bicyclic or polycyclic ring having, in addition to the atom, 0-10 heteroatoms; or two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
391. The compound of Embodiment 390, wherein X^{M} is —S—.
392. The compound of Embodiment 390, wherein X^{M} is —NR^{MN}—.
393. The compound of any one of Embodiments 390-392, wherein Ring M is 5-membered.
394. The compound of any one of Embodiments 390-392, wherein Ring M is 6-membered.
395. The compound of any one of Embodiments 390-394, wherein Ring M is monocyclic.
396. The compound of any one of Embodiments 390-394, wherein Ring M is bicyclic.
397. The compound of any one of Embodiments 390-396, wherein Ring M is saturated.
398. The compound of any one of Embodiments 390-397, wherein Ring M has no heteroatoms in addition to the intervening atoms.
399. The compound of any one of Embodiments 390-398, wherein t is 1-10.
400. The compound of any one of Embodiments 390-399, wherein t is 2.
401. The compound of any one of Embodiments 390-400, wherein each R^{M1} is independently R.
402. The compound of any one of Embodiments 390-400, wherein each R^{M1} is independently optionally substituted C₁₋₃₀ aliphatic.
403. The compound of any one of Embodiments 390-402, wherein

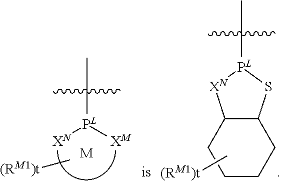

404. The compound of any one of Embodiments 390-402, wherein

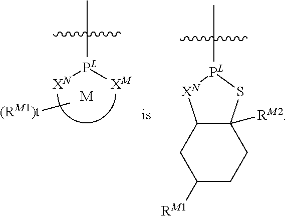

405. The compound of any one of Embodiments 390-402, wherein

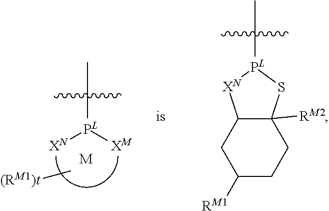

wherein R^{M1} and R^{M2}are trans.
406. The compound of any one of Embodiments 390-402, wherein

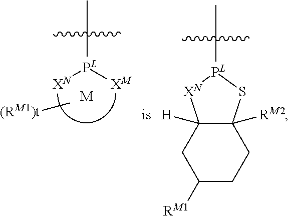

wherein the H and R^{M2} are trans.
407. The compound of any one of Embodiments 390-402, wherein

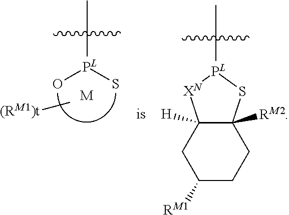

408. The compound of any one of Embodiments 390-402, wherein

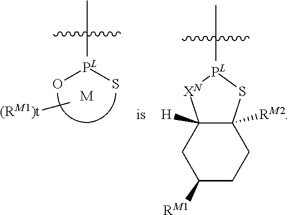

409. The compound of any one of Embodiments 404-408, wherein R^{M1} is —C(CH₃)═CH₂.
410. The compound of any one of Embodiments 404-409, wherein R^{M2} is —CH₃.
411. The compound of any one of Embodiments 390-402, wherein

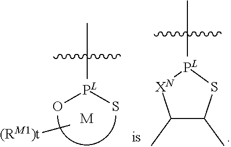

412. The compound of any one of Embodiments 392-401, wherein one R^{M1} and R^{MN} are taken together with their intervening atoms to form an optionally substituted 3-30 membered, monocyclic, bicyclic or polycyclic ring having 0-10 heteroatoms.
413. The compound of Embodiment 412, wherein the formed ring is monocyclic.
414. The compound of any one of Embodiments 412-413, wherein the formed ring is 5-membered.
415. The compound of any one of Embodiments 412-414, wherein the formed ring comprises no heteroatoms in addition to the intervening atoms.
416. The compound of any one of Embodiments 412-415, wherein the formed ring is saturated.
417. The compound of any one of Embodiments 412-416, wherein the formed ring is fused with Ring M.
418. The compound of any one of Embodiments 390-417, wherein LG is —Cl.
419. The compound of any one of Embodiments 390-417, wherein LG is —N(R′)₂.
420. The compound of any one of Embodiments 390-417, wherein LG is —N(iPr)₂.
421. A compound having the structure of formula M-I:

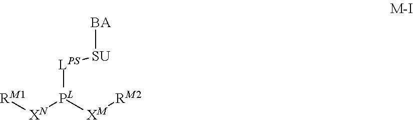

or a salt thereof, wherein:

each of X^{M} and X^{N} is independently -L-O—, -L-S— or -L-NR^{MN}—;

P^{L} is P, P(═W), P->B(-L^{L}-R^{L})₃, or P^{N};

W is O, N(-L^{L}-R^{L}), S or Se;

P^{N} is P═N—C(-L^{L}-R′)(=L^{N}-R′) or P═N-L^{L}-R^{L};

L^{N} is =N-L^{L1}-, ═CH-L^{L1}- wherein CH is optionally substituted, or ═N⁺(R′)(Q⁻)-L^{L1}-;

each L^{L1} is independently L;

Q⁻ is an anion;

each of R^{M1}, R^{M2} and R^{MN} is independently -L^{M}-R^{M};

each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

each R^{L} is independently -L^{L}-R′ or —N═C(-L^{L}-R′)₂;

each of L^{L} and L^{M} is independently L;

BA is a nucleobase;

SU is a sugar;

L^{PS} is a L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted, 3-30 membered, monocyclic, bicyclic or polycyclic ring having 0-10 heteroatoms;

each Cy^{L} is independently an optionally substituted, trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
422. The compound of Embodiment 421, wherein X^{M} is —5—.
423. The compound of Embodiment 421, wherein X^{M} is —NR^{MN}—.
424. The compound of any one of Embodiments 421-423, wherein R^{M1} and R^{Ma}are taken together with their intervening atoms to form an optionally substituted, 3-30 membered, monocyclic, bicyclic or polycyclic ring having, in addition to the intervening atoms, 0-10 heteroatoms.
425. The compound of Embodiment 424, wherein the formed ring is 5-membered.
426. The compound of Embodiment 424, wherein the formed ring is 6-membered.
427. The compound of any one of Embodiments 421-426, wherein the formed ring is substituted.
428. The compound of any one of Embodiments 421-427, wherein the formed ring is monocyclic.
429. The compound of any one of Embodiments 421-427, wherein the formed ring is bicyclic.
430. The compound of any one of Embodiments 421-429, wherein the formed ring has no heteroatoms in addition to the intervening atoms.
431. The compound of Embodiment 423, wherein R^{M1}, R^{M2} and R^{MN} are taken together with their intervening atoms to form an optionally substituted 3-30 membered, monocyclic, bicyclic or polycyclic ring having 0-10 heteroatoms.
432. The compound of Embodiment 431, wherein the formed ring is an optionally substituted 8-10 membered bicyclic ring.
433. The compound of any one of Embodiments 431-432, wherein the formed ring is an optionally substituted [5,5]-fused bicyclic ring.
434. The compound of any one of Embodiments 431-433, wherein the formed ring comprise no heteroatoms in addition to the intervening atoms.
435. The compound of any one of Embodiments 431-434, wherein the formed ring is saturated.
436. A compound having the structure of formula M-II:

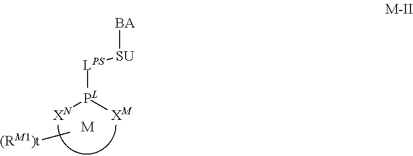

or a salt thereof, wherein:

each of X^{M} and X^{N} is independently -L-O—, -L-S— or -L-NR′ ^{N}—; P^{L} is P, P(═W), P->B(-L^{L}-R^{L})₃, or P^{N}; W is O, N(-L^{L}-R^{L}), S or Se; P^{N} is P═N—C(-L^{L}-R′)(=L^{N}—R′) or P═N-L^{L}-R^{L}; L^{N} is ═N-L″—,=CH-L″—wherein CH is optionally substituted, or=N⁺(R′)(Q⁻)-L″—; each L″ is independently L; Q⁻ is an anion; each of R^{M1} and R^{MN} is independently -L^{M}—R^{M}; each R^{L} is independently -L^{L}-R′ or N═C(L^{L} R′)₂; each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

t is 0-10; each of L^{L} and L″ is independently L; Ring M is an optionally substituted 3-30 membered, monocyclic, bicyclic or polycyclic ring having 0-10 heteroatoms;

BA is a nucleobase; SU is a sugar; L^{PS} is a L; each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C E C a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′))O——OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R; each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or two R groups are optionally and independently taken together to form a covalent bond, or: two or more R groups on the same atom are optionally and independently taken together with the atom to form an optionally substituted, 3-30 membered, monocyclic, bicyclic or polycyclic ring having, in addition to the atom, 0-10 heteroatoms; or two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
437. The compound of Embodiment 436, wherein X^{M} is —S—.
438. The compound of Embodiment 436, wherein X^{M} is —NR^{MN}—.
439. The compound of any one of Embodiments 436-438, wherein Ring M is 5-membered.
440. The compound of any one of Embodiments 436-438, wherein Ring M is 6-membered.
441. The compound of any one of Embodiments 436-440, wherein Ring M is monocyclic.
442. The compound of any one of Embodiments 436-440, wherein Ring M is bicyclic.
443. The compound of any one of Embodiments 436-442, wherein Ring M is saturated.
444. The compound of any one of Embodiments 436-443, wherein Ring M has no heteroatoms in addition to the intervening atoms.
445. The compound of any one of Embodiments 436-444, wherein t is 1-10.
446. The compound of any one of Embodiments 436-445, wherein t is 2.
447. The compound of any one of Embodiments 436-446, wherein each R^{M1} is independently R.
448. The compound of any one of Embodiments 436-447, wherein each R^{M1} is independently optionally substituted C₁₋₃₀ aliphatic.

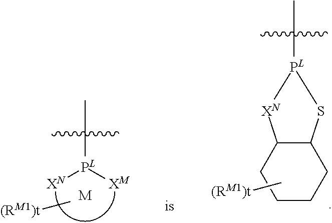

449. The compound of any one of Embodiments 436-448, wherein

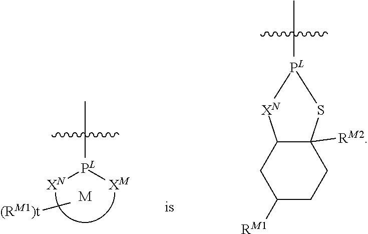

450. The compound of any one of Embodiments 436-448, wherein

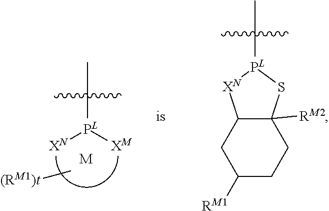

451. The compound of any one of Embodiments 436-448, wherein wherein R^{M1} and R^{M2}are trans.

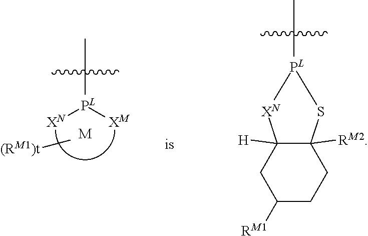

452. The compound of any one of Embodiments 436-448, wherein wherein the H and R^{M2} are trans.

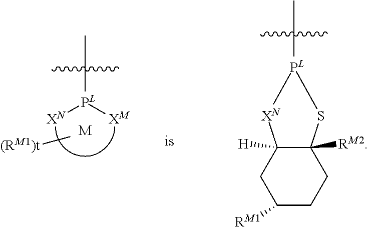

453. The compound of any one of Embodiments 436-448, wherein

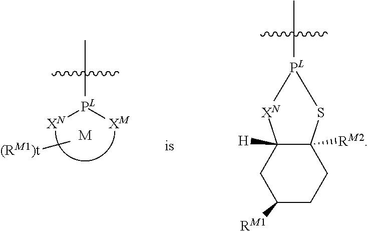

454. The compound of any one of Embodiments 436-448, wherein
455. The compound of any one of Embodiments 450-454, wherein R^{M1} is —C(CH₃)═CH₂.
456. The compound of any one of Embodiments 450-455, wherein R^{M2} is —CH₃.

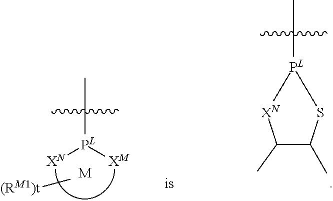

457. The compound of any one of Embodiments 436-448, wherein
458. The compound of any one of Embodiments 438-447, wherein one R^{M1} and O^{N} are taken together with their intervening atoms to form an optionally substituted 3-30 membered, monocyclic, bicyclic or polycyclic ring having 0-10 heteroatoms.
459. The compound of Embodiment 458, wherein the formed ring is monocyclic.
460. The compound of any one of Embodiments 458-459, wherein the formed ring is 5-membered.
461. The compound of any one of Embodiments 458-460, wherein the formed ring comprises no heteroatoms in addition to the intervening atoms.
462. The compound of any one of Embodiments 458-461, wherein the formed ring is saturated.
463. The compound of any one of Embodiments 458-462, wherein the formed ring is fused with Ring

M.

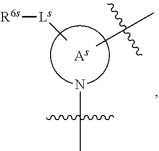

464. The compound of any one of Embodiments 367-463, wherein SU is wherein:

R⁶⁵ is R⁵; each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

Ring A^{s} is an optionally substituted 3-30 membered, monocyclic, bicyclic or polycyclic ring having, in addition to the nitrogen, 0-10 heteroatoms;

L^{s} is L; each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C═C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R; each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or two R groups are optionally and independently taken together to form a covalent bond, or: two or more R groups on the same atom are optionally and independently taken together with the atom to form an optionally substituted, 3-30 membered, monocyclic, bicyclic or polycyclic ring having, in addition to the atom, 0-10 heteroatoms; or two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms. 465. The compound of Embodiment 464, wherein the N is bonded to P^{L}. 466. The compound of any one of Embodiments 464-465, wherein L^{s} is —C(R^{5s})₂, wherein each R^{5s} is independently R^{s}. 467. The compound of any one of Embodiments 464-466, wherein L^{s} is optionally substituted —CH₂—. 468. The compound of any one of Embodiments 464-467, wherein L^{s} is —CH₂—.

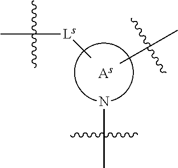

469. The compound of any one of Embodiments 464-468, wherein is optionally

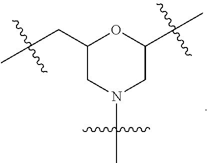 [SMILES: *CC1CN(*)CC(*)O1]

substituted

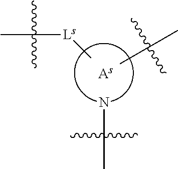

470. The compound of any one of Embodiments 464-468, wherein is optionally

substituted

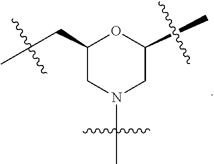

471. The compound of any one of Embodiments 464-468, wherein

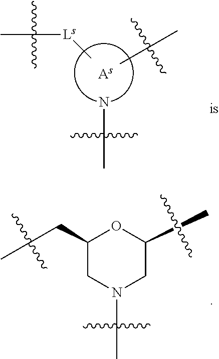

472. The compound of any one of Embodiments 464-468, wherein

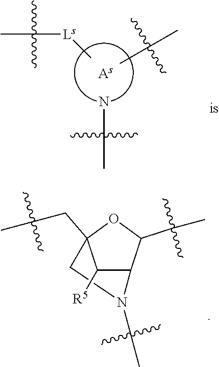

473. The compound of any one of Embodiments 464-468, wherein

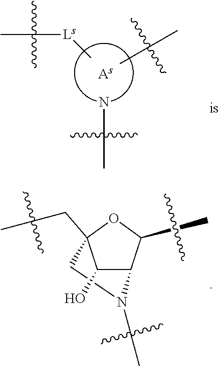

474. The compound of any one of Embodiments 464-468, wherein

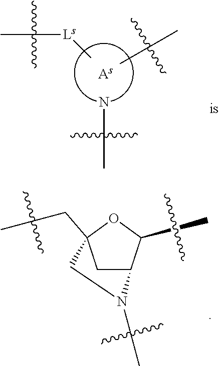

475. The compound of any one of Embodiments 367-457, wherein SU is

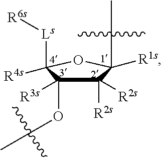

wherein:

each of R^{1s}, R^{2s}, R^{3s}, R^{4s}, R^{5s}, and R^{6s} is independently R^{s};

each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

L^{s} is L; each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
476. The compound of Embodiment 475, wherein Cl is bonded to BA.
477. The compound of any one of Embodiments 475-476, wherein L^{s} is —C(R^{5s})₂, wherein each R^{5s} is independently R^{s}.
478. The compound of any one of Embodiments 475-477, wherein L^{s} is optionally substituted —CH₂—.
479. The compound of any one of Embodiments 475-478, wherein L^{s} is —CH₂—.
480. The compound of any one of Embodiments 475-479, wherein R^{1s} is —H.
481. The compound of any one of Embodiments 475-480, wherein R^{3s} is —H.
482. The compound of any one of Embodiments 475-481, wherein SU is

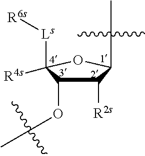

483. The compound of any one of Embodiments 475-482, wherein R^{2s} is —H.
484. The compound of any one of Embodiments 475-482, wherein R^{2s} is —F.
485. The compound of any one of Embodiments 475-482, wherein R^{2s} is —OR, wherein R is C₁-₆ aliphatic.
486. The compound of any one of Embodiments 475-482, wherein R^{2s} is —OMe.
487. The compound of any one of Embodiments 475-482, wherein R^{2s} is -MOE.
488. The compound of any one of Embodiments 475-487, wherein R^{4s} is —H.
489. The compound of any one of Embodiments 475-487, wherein R^{2s} and R^{4s} are taken together to form-L-.
490. The compound of any one of Embodiments 475-487, wherein R^{2s} and R^{4s} are taken together to form-L-, wherein L is 2′-O—CH₂-4′, wherein the —CH₂— is optionally substituted.
491. The compound of any one of Embodiments 367-457, wherein SU is -L^{PS}—SU′- R⁶′, wherein SU′ is an acyclic sugar.
492. The compound of Embodiment 491, wherein L^{PS} is —O—.
493. The compound of Embodiment 491, wherein L^{PS} is —NR′—.
494. The compound of Embodiment 491, wherein L^{PS} is a covalent bond.
495. The compound of any one of Embodiments 464-494, wherein R^{6s} is —O-L-R′.
496. The compound of any one of Embodiments 464-494, wherein R^{6s} is —OH protected for oligonucleotide synthesis.
497. The compound of any one of Embodiments 464-494, wherein R^{6s} is DMTrO—.
498. The compound of any one of the preceding Embodiments, wherein X^{N} is —O—.
499. The compound of any one of the preceding Embodiments, wherein X^{N} is —S—.
500. The compound of any one of the preceding Embodiments, wherein X^{N} is —NR^{MN}—.
501. The compound of any one of Embodiments 371-500, wherein P^{L} is P.
502. The compound of any one of Embodiments 371-500, wherein P^{L} is P(═W), wherein W is O.
503. The compound of any one of Embodiments 371-500, wherein P^{L} is P(═W), wherein W is S
504. The compound of any one of Embodiments 371-500, wherein P^{L} is P(═W), wherein W is Se.
505. The compound of any one of Embodiments 371-500, wherein P^{L} is P(═W), wherein W is N(-L^{L}-R^{L}).
506. The compound of any one of Embodiments 371-500, wherein P^{L} is P^{N}.
507. The compound of Embodiment 506, wherein P^{L} is P═N—C(-L^{L}-R′)[═N⁺(R′)(Q⁻)-L^{L1}-R′].
508. The compound of Embodiment 506, wherein P^{L} is P═N—C(-L^{L}-R′)[═N⁺(R′)(Q⁻)-R′)].
509. The compound of Embodiment 506, wherein P^{L} is P═N—C[N(R′)₂][=N⁺(R′)(Q⁻)-L^{L1}-R′].
510. The compound of Embodiment 506, wherein P^{L} is P═N—C[N(R′)₂][=N⁺(R′)₂(Q⁻)].
511. The compound of any one of Embodiments 509-510, wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms.
512. The compound of Embodiment 511, wherein one R′ on one N and one R′ or the other N are taken together with their intervening atoms to form an optionally substituted, 5-membered, monocyclic, bicyclic or polycyclic ring having, in addition to the intervening atoms, 0 heteroatoms.
513. The compound of any one of Embodiments 511-512, wherein the formed ring is saturated.
514. The compound of any one of Embodiments 506-513, wherein P^{N} is

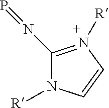

### Q⁻.

515. The compound of any one of Embodiments 506-516, wherein P^{N} is

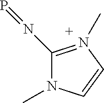

### Q⁻.

516. The compound of any one of Embodiments 506-513, wherein P^{N} is

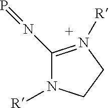

### Q⁻.

517. The compound of any one of Embodiments 506-516, wherein P^{N} is

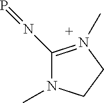

### Q⁻.

518. The compound of any one of the preceding Embodiments, wherein Q⁻ is PF₆⁻.
519. A compound having the structure of formula M-III:

BA-SU-C(O)-LG^{M}, M-III

or a salt thereof, wherein:

BA is a nucleobase;

SU is a sugar; and

LG^{M} is a leaving group.
520. The compound of Embodiment 519, wherein LG^{M} is optionally substituted heteroaryl.
521. The compound of Embodiment 520, wherein LG^{M} is optionally substituted

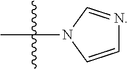 [SMILES: *n1ccnc1]

522. The compound of Embodiment 521, wherein LG^{M} is

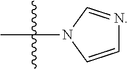 [SMILES: *n1ccnc1]

523. The compound of Embodiment 520, wherein LG^{M} is optionally substituted

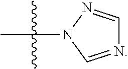 [SMILES: *n1cncn1]

524. The compound of Embodiment 523, wherein LG^{M} is

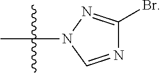 [SMILES: *n1cnc(Br)n1]

525. The compound of any one of Embodiments 519-524, wherein SU is

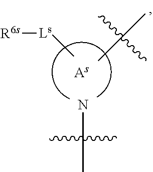

wherein:

R^{6s} is R^{s};

each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

Ring A^{s} is an optionally substituted 3-30 membered, monocyclic, bicyclic or polycyclic ring having, in addition to the nitrogen, 0-10 heteroatoms;

L^{s} is L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
526. The compound of Embodiment 525, wherein the N is bonded to —C(O)—R^{M}.
527. The compound of any one of Embodiments 525-526, wherein L^{s} is —C(R^{5s})₂, wherein each R^{5s} is independently R^{s}.
528. The compound of any one of Embodiments 525-527, wherein L^{s} is optionally substituted —CH₂—.
529. The compound of any one of Embodiments 525-528, wherein L^{s} is —CH₂—.
530. The compound of any one of Embodiments 525-529, wherein

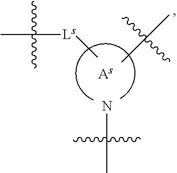

is optionally substituted

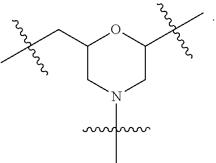 [SMILES: *CC1CN(*)CC(*)O1]

531. The compound of any one of Embodiments 525-530, wherein

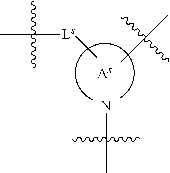

is optionally
substituted

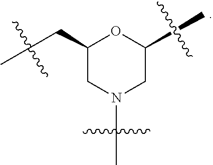

532. The compound of any one of Embodiments 525-531, whereir

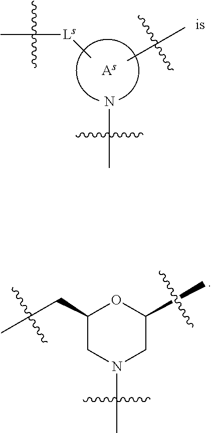

533. The compound of any one of Embodiments 525-531, wherein

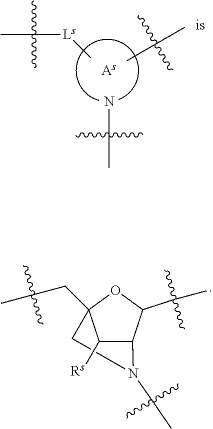

534. The compound of any one of Embodiments 525-531, wherein

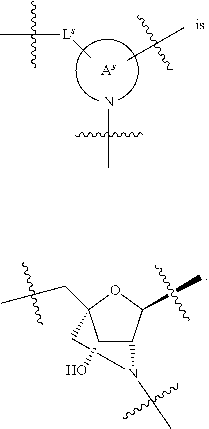

535. The compound of any one of Embodiments 525-531, wherein

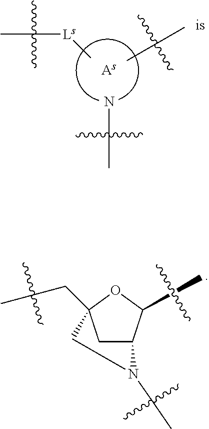

536. The compound of any one of Embodiments 519-524, wherein SU is

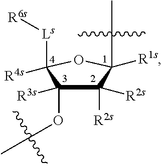

wherein:

each of R^{1s}, R^{2s}, R^{3s}, R^{4s}, R^{5s}, and R^{6s} is independently R^{s};

each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

L^{s} is L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R; each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
537. The compound of Embodiment 536, wherein Cl is bonded to BA.
538. The compound of any one of Embodiments 536-537, wherein L^{s} is —C(R^{5s})₂, wherein each R^{5s} is independently R^{s}.
539. The compound of any one of Embodiments 536-538, wherein L^{s} is optionally substituted —CH₂—.
540. The compound of any one of Embodiments 536-539, wherein L^{s} is —CH₂—.
541. The compound of any one of Embodiments 536-540, wherein R^{1s} is —H.
542. The compound of any one of Embodiments 536-541, wherein R^{3s} is —H.
543. The compound of any one of Embodiments 536-542, wherein SU is

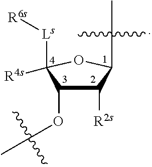

544. The compound of any one of Embodiments 536-543, wherein R^{2s} is —H.
545. The compound of any one of Embodiments 536-543, wherein R^{2s} is —F.
546. The compound of any one of Embodiments 536-543, wherein R^{2s} is —OR, wherein R is C₁₋₆ aliphatic.
547. The compound of any one of Embodiments 536-543, wherein R^{2s} is —OMe.
548. The compound of any one of Embodiments 536-543, wherein R^{2s} is -MOE.
549. The compound of any one of Embodiments 536-548, wherein R^{4s} is —H.
550. The compound of any one of Embodiments 536-548, wherein R^{2s} and R^{4s} are taken together to form-L-.
551. The compound of any one of Embodiments 536-548, wherein R^{2s} and R^{4s} are taken together to form-L-, wherein L is 2′-O—CH₂-4′, wherein the —CH₂— is optionally substituted.
552. The compound of any one of Embodiments 519-524, wherein SU is -L^{PS}-SU′13 R^{6s}, wherein SU′ is an acyclic sugar.
553. The compound of Embodiment 552, wherein L^{PS} is —O—.
554. The compound of Embodiment 552, wherein L^{PS} is —NR′—.
555. The compound of Embodiment 552, wherein L^{PS} is a covalent bond.
556. The compound of any one of Embodiments 519-555, wherein R^{6s} is —O-L-R′.
557. The compound of any one of Embodiments 519-556, wherein R^{6s} is —OH protected for oligonucleotide synthesis.
558. The compound of any one of Embodiments 519-557, wherein R^{6s} is DMTrO—.
559. A method for preparing a compound of any one of Embodiments 371-420, comprising contacting a compound of Embodiments 364-370 with a second compound.
560. The method of Embodiment 559, wherein the second compound is PC13.
561. A method, comprising a coupling step that comprises:

contacting a first compound with a second compound comprising a hydroxyl group or an amino group, wherein the first compound is a compound any one of Embodiments 502-558.
562. A method, comprising a coupling step that comprises:

contacting a first compound with a second compound comprising a hydroxyl group or an amino group in the presence of a base, wherein the first compound is a compound any one of Embodiments 506-518.
563. The method of Embodiment 561, wherein the P of the P^{N} in the first compound forms a bond with the 0 of the —OH of the second compound.
564. A method, comprising a coupling step that comprises:

contacting a first composition with a second compound comprising a hydroxyl group or an amino group, wherein the first composition is prepared by a method comprising contacting a compound of Embodiment 501 with a compound of formula AZ-1:

N₃—C(-L^{L}-R′)[═N⁺(R′)(Q⁻)-L^{L1}-R′]. AZ-I
565. The method of Embodiment 561, wherein a first compound is prepared by contacting a compound of Embodiment 501 with a compound having the structure of AZ-1:

N₃—C(-L^{L}-R′)[═N⁺(R′)(Q⁻)-L^{L1}-R′]. AZ-I
566. The method of Embodiment 561, wherein a first compound is utilized without isolation and/or purification.
567. The method of any one of Embodiments 564-566, wherein a compound of formula AZ-I is a compound of formula N₃—C(-L^{L}-R′)[═N⁺(R′)(Q⁻)-R′].
568. The method of any one of Embodiments 564-566, wherein a compound of formula AZ-I is a compound of formula N₃—C[N(R′)₂][═N⁺(R′)(Q⁻)-L^{L1}-R′].
569. The method of any one of Embodiments 564-566, wherein a compound of formula AZ-I is a compound of formula N₃—C[N(R′)₂][═N⁺(R′)₂(Q⁻)].
570. The method of any one of Embodiments 564-566, wherein a compound of formula AZ-I is a compound of formula N₃—C(-L^{L}-R′)[═N⁺(R′)₂(Q⁻)].
571. The method of any one of Embodiments 564-570, wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms.
572. The method of Embodiment 571, wherein one R′ on one N and one R′ or the other N are taken together with their intervening atoms to form an optionally substituted, 5-membered, monocyclic, bicyclic or polycyclic ring having, in addition to the intervening atoms, 0 heteroatoms.
573. The method of any one of Embodiments 571-572, wherein the formed ring is saturated.
574. The method of any one of Embodiments 571-573, wherein a compound of formula AZ-1 is

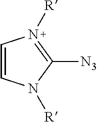

### Q⁻.

575. The method of any one of Embodiments 571-576, wherein a compound of formula AZ-1 is

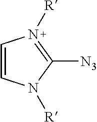

### Q⁻.

576. The method of any one of Embodiments 571-573, wherein a compound of formula AZ-1 is

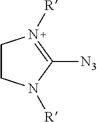

### Q⁻.

577. The method of any one of Embodiments 571-576, wherein a compound of formula AZ-1 is

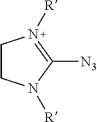

### Q⁻.

578. The method of any one of Embodiments 571-577, wherein Q⁻ is PF₆⁻.
579. The method of any one of Embodiments 561-578, wherein the contacting produces a third compound comprising —P(═W)(—X-L^{L}-R¹)—Z—.
580. The method of Embodiment 579, wherein Z is —O—.
581. The method of any one of Embodiments 579-580, wherein W is O.
582. The method of any one of Embodiments 579-581, wherein an occurrence of X is a covalent bond, and R^{L} is —N═C(-L^{L}-R′)₂.
583. The method of any one of Embodiments 579-582, wherein an occurrence of -L^{L}- is —N(R′)—.
584. The method of any one of Embodiments 579-583, wherein R^{L} is —N═C[N(R′)₂]₂.
585. The method of Embodiment 584, wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms.
586. The method of Embodiment 584, wherein one R′ on one N and one R′ or the other N are taken together with their intervening atoms to form an optionally substituted, 5-membered, monocyclic, bicyclic or polycyclic ring having, in addition to the intervening atoms, 0 heteroatoms.
587. The method of any one of Embodiments 585-586, wherein the formed ring is saturated.
588. The method of any one of Embodiments 579-587, wherein R^{L} is

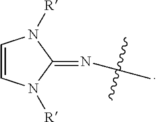

589. The method of any one of Embodiments 579-590, wherein R^{L}

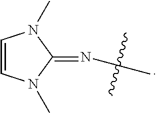 [SMILES: *N=c1n(C)ccn1C]

590. The method of any one of Embodiments 579-587, wherein R^{L} is

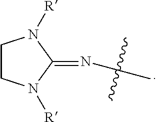

591. The method of any one of Embodiments 579-590, wherein R^{L} is

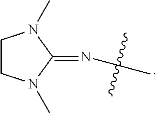 [SMILES: *N=C1N(C)CCN1C]

592. The method of any one of Embodiments 579-591, wherein the P of —P(═W)(—X-L^{L}-R^{L})—Z— is the P of the P^{N} of the first compound.
593. The method of any one of Embodiments 561-592, comprising converting P^{L} in a first compound or composition which P^{L} is P═N—C(-L^{L}-R′)[═N⁺(R′)(Q⁻)-L^{L1}-R′] into P—N═C(-L^{L}-R′)[N(R′)₂].
594. The method of any one of Embodiments 561-592, comprising converting P^{L} in a first compound or composition which P^{L} is P═N—C(-L^{L}-R′)[═N⁺(R′)(Q⁻)-R′] into P—N═C(-L^{L}-R′)[—N(R′)₂].
595. The method of any one of Embodiments 561-592, comprising converting P^{L} in a first compound or composition which P^{L} is P═N—C[N(R′)_{2][═N}⁺(R′)(Q⁻)-L^{L1}-R′] into P═N—C[N(R′)₂][—N(R′)-L^{L1}-R′].
596. The method of any one of Embodiments 561-592, comprising converting P^{L} in a first compound or composition which P^{L} is P═N—C[N(R′)₂][═N⁺(R′)₂(Q⁻)] into P═N—C[N(R′)₂][—N(R′)₂].
597. The method of any one of Embodiments 593-596, wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms.
598. The method of Embodiment 597, wherein one R′ on one N and one R′ or the other N are taken together with their intervening atoms to form an optionally substituted, 5-membered, monocyclic, bicyclic or polycyclic ring having, in addition to the intervening atoms, 0 heteroatoms.
599. The method of any one of Embodiments 597-598, wherein the formed ring is saturated.
600. The method of any one of Embodiments 593-599, comprising converting

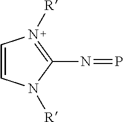

into

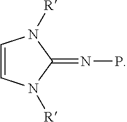

601. The method of any one of Embodiments 593-602, comprising converting

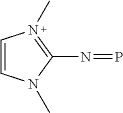

into

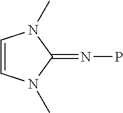

602. The method of any one of Embodiments 593-599, comprising converting

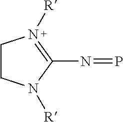

into

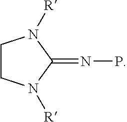

603. The method of any one of Embodiments 593-602, comprising converting

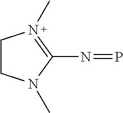

into

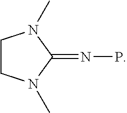

604. The method of any one of Embodiments 593-603, wherein Q⁻ is PF₆⁻.
605. A method, comprising a coupling step that comprises:

contacting a first compound with a second compound comprising a hydroxyl group or amino group, wherein the first compound is a compound of Embodiment 503.
606. The method of Embodiment 561, wherein the P of the P═S in the first compound forms a bond with the O of the —OH or the N or the amino group of the second compound.
607. A method, comprising a coupling step that comprises:

contacting a first composition with a second compound comprising a hydroxyl group or an amino group, wherein the first composition is prepared by a method comprising contacting a compound of Embodiment 501 with sulfurization agent.
608. The method of Embodiment 607, wherein a first compound is prepared by contacting a compound of Embodiment 501 with a sulfurization agent.
609. The method of Embodiment 608, wherein a first compound is utilized without isolation and/or purification.
610. The method of any one of Embodiments 607-609, wherein a sulfurization agent is

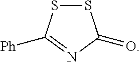 [SMILES: O=c1nc(-c2ccccc2)ss1]

611. The method of any one of Embodiments 605-610, wherein the contacting produces a third compound comprising —P(═W)(—X-L^{L}-R¹)—Z—.
612. The method of Embodiment 611, wherein Z is —O—.
613. The method of any one of Embodiments 611-612, wherein W is O.
614. The method of any one of Embodiments 611-613, wherein —X-L^{L}-R^{L} is —S—H.
615. A method, comprising a coupling step that comprises:

contacting a first compound with a second compound comprising a hydroxyl or amino group, wherein the first compound is a compound of any one of Embodiment 519-558.
616. The method of any one of Embodiments 561-615, wherein the second compound is or comprises a nucleoside comprising a —OH group.
617. The method of any one of Embodiments 561-618, wherein the second compound is or comprises an oligonucleotide comprising a —OH group.
618. The method of any one of Embodiments 561-615, wherein the second compound is or comprises a nucleoside comprising an amino group.
619. The method of any one of Embodiments 561-618, wherein the second compound is or comprises an oligonucleotide comprising an amino group.
620. The method of any one of Embodiments 564-619, wherein the nucleoside or oligonucleotide is or comprises

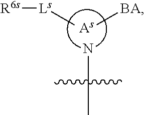

wherein:

R^{6s} is —OH;

Ring A^{s} is an optionally substituted 3-30 membered, monocyclic, bicyclic or polycyclic ring having, in addition to the nitrogen, 0-10 heteroatoms;

each R⁵ is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

L^{s} is L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)O—, —OP(SR′)—, —OP(NR′)—, —OP(R′)—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
621. The method of Embodiment 620, wherein L^{s} is —C(R^{5s})₂, wherein each R^{5s} is independently R^{s}.
622. The method of any one of Embodiments 620-621, wherein L^{s} is optionally substituted —CH₂—.
623. The method of any one of Embodiments 620-622, wherein L^{s} is —CH₂—.
624. The method of any one of Embodiments 620-623, wherein

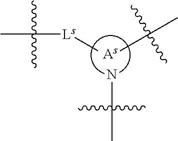

is optionally substituted

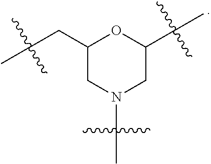 [SMILES: "." ; *CC1CN(*)CC(*)O1]

625. The method of any one of Embodiments 620-624, wherein

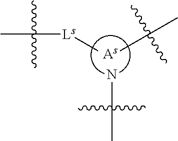

is optionally substituted

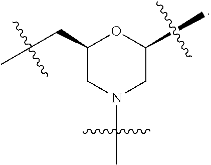

626. The method of any one of Embodiments 620-625, wherein

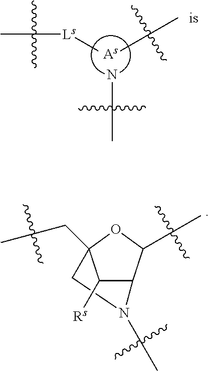

627. The method of any one of Embodiments 620-625, wherein

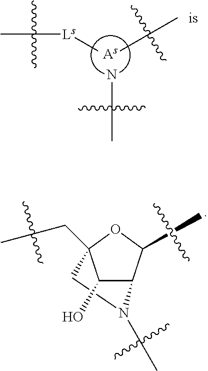

628. The method of any one of Embodiments 620-625, wherein

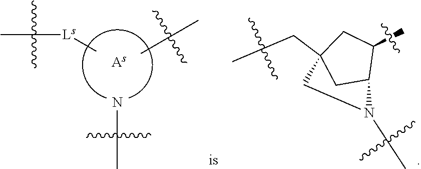

629. The method of any one of Embodiments 620-625, wherein

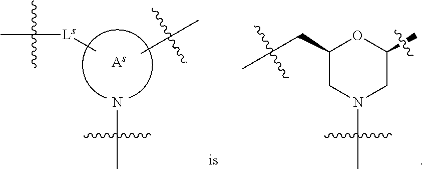

630. The method of any one of Embodiments 564-619, wherein the nucleoside or oligonucleotide is or comprises

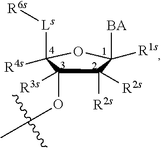

wherein:

R^{6s} is —OH;

each of R^{1s}, R^{2s}, R^{3s}, R^{4s}, and R^{5s} is independently R^{s};

each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

L^{s} is L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)O—, —OP(SR′)—, —OP(NR′)—, —OP(R′)—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
631. The method of Embodiment 630, wherein L^{s} is —C(R^{5s})₂, wherein each R^{5s} is independently R^{s}.
632. The method of any one of Embodiments 630-631, wherein L^{s} is optionally substituted —CH₂—.
633. The method of any one of Embodiments 630-632, wherein L^{s} is —CH₂—.
634. The method of any one of Embodiments 630-633, wherein R^{1s} is —H.
635. The method of any one of Embodiments 630-634, wherein R^{3s} is —H.
636. The method of any one of Embodiments 630-635, wherein the nucleoside or oligonucleotide is or comprises

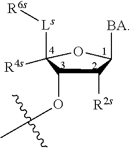

637. The method of any one of Embodiments 630-636, wherein R^{2s} is —H.
638. The method of any one of Embodiments 630-636, wherein R^{2s} is —F.
639. The method of any one of Embodiments 630-636, wherein R^{2s} is —OR, wherein R is C₁-₆ aliphatic.
640. The method of any one of Embodiments 630-636, wherein R^{2s} is —OMe.
641. The method of any one of Embodiments 630-636, wherein R^{2s} is -MOE.
642. The method of any one of Embodiments 630-641, wherein R^{4s} is —H.
643. The method of any one of Embodiments 630-641, wherein R^{2s} and R^{4s} are taken together to form-L-.
644. The method of any one of Embodiments 630-641, wherein R^{2s} and R^{4s} are taken together to form-L-, wherein L is 2′-O—CH₂-4′, wherein the —CH₂— is optionally substituted.
645. The method of any one of Embodiments 564-619, wherein SU is -L^{PS}—SU′—R^{6s}, wherein SU′ is an acyclic sugar.
646. The method of Embodiment 645, wherein L^{PS} is —O—.
647. The method of Embodiment 645, wherein L^{PS} is —NR′—.
648. The method of Embodiment 645, wherein L^{PS} is a covalent bond.
649. The method of any one of the preceding Embodiments, wherein R^{6s} is —O-L-R′.
650. The method of any one of the preceding Embodiments, wherein R^{6s} is —OH protected for oligonucleotide synthesis.
651. The method of any one of the preceding Embodiments, wherein R^{6s} is DMTrO—.
652. The method of any one of Embodiments 561-651, wherein the second compound is linked to a solid support optionally through a linker.
653. The method of Embodiment 652, wherein the second compound is a nucleoside or an oligonucleotide linked to a solid support through a linker.
654. The method of any one of Embodiments 652-653, wherein the linker comprises one or more —N(R′)—, wherein R′ is not —H.
655. The method of any one of Embodiments 652-653, wherein the linker comprises one or more —(CH₂)m—N(R′)—C(O)—(CH₂)n—C(O), wherein R′ is not —H, each of m and n is independently 1-20, and each —CH₂— is independent optionally substituted.
656. The method of any one of Embodiments 652-653, wherein the linker comprises one or more —(CH₂)m—N(R′)—C(O)—(CH₂)₂—C(O), wherein R′ is not —H, each of m and n is independently 1-20, and each —CH₂— is independent optionally substituted.
657. The method of any one of Embodiments 652-653, wherein the linker comprises one or more —N(R′)—, wherein R′ is optionally substituted C₁₋₆ aliphatic.
658. The method of any one of Embodiments 652-653, wherein the linker comprises one or more —N(R′)—, wherein R′ is methyl.
659. The method of any one of Embodiments 652-658, wherein the linker comprises no —NH—.
660. The method of any one of Embodiments 652-659, wherein the solid support is CPG.
661. The method of any one the preceding Embodiments, wherein the contacting is performed in the presence of a base.
662. The method of Embodiment 661, wherein the base is DBU.
663. The method of any one of Embodiments 561-662, comprising a capping step that comprises a condition under which a —OH group can be capped.
664. The method of any one of Embodiments 561-663, comprising a capping step that comprises: contacting a product of a coupling step with a capping composition comprising a compound having the structure of [R′C(O)]₂.
665. The method of any one of Embodiments 604-663, wherein a capping step comprises contacting a product of a coupling step with a capping composition comprising Ac₂O.
666. The method of any one of Embodiments 561-665, comprising a deprotection step that comprises a condition under which a protected —OH group can be deprotected.
667. The method of any one of Embodiments 561-666, comprising a deprotection step that comprises:

contacting a product of a coupling or a capping step with a deprotection composition comprising an acid.
668. The method of any one of Embodiments 561-666, comprising a deprotection step that comprises:

contacting a product of a coupling or a capping step with a deprotection composition comprising an acid, wherein a DMTrO— is converted into —OH.
669. A method, comprising one or more cycles each independently comprising:

a) a coupling step,

b) a capping step, and

c) optionally a deprotection step,

wherein each of the coupling step, capping step, and deprotection step is independently as described in any one of Embodiments 561-667.
670. The method of Embodiment 669, wherein at least one cycle comprise a deprotection step.
671. The method of Embodiment 669, wherein each one of the one or more cycles independently comprise a deprotection step.
672. The method of any one of Embodiments 669-671, wherein the cycle comprise no steps that modify the linkage phosphorus.
673. The method of any one of Embodiments 669-672, wherein the coupling step comprises coupling with a compound of any one of Embodiments 502-558.
674. The method of any one of Embodiments 564-673, further comprising one or more cycles each of which independently comprising:

a) a coupling step,

b) optionally a first capping step,

c) a modification step,

d) optionally a second capping step, and

e) optionally a deprotection step,
675. The method of Embodiment 674, wherein the coupling step comprises coupling with a compound of Embodiment 501.
676. The method of any one of Embodiments 674-675, comprising a first capping step which comprises an amidation condition.
677. The method of any one of Embodiments 674-676, comprising a modification step which modifies a linkage phosphorus form P to P(═O), P═S or P═N—.
678. The method of any one of Embodiments 674-677, comprising a second capping step which comprises an esterification condition.
679. A method for preparing a compound of Embodiment 501, comprising contacting a compound of any one of Embodiments 367-418 with a nucleoside.
680. The method of Embodiment 679, wherein the nucleoside is A, T, C or G, optionally protected for oligonucleotide synthesis.
681. The method of Embodiment 679, wherein the nucleoside is of the structure H—SU—BA or a salt thereof.
682. The method of Embodiment 681, wherein SU is

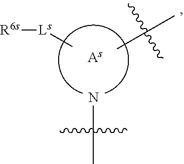

wherein:

R^{6s} is R^{s};

each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

Ring A^{s} is an optionally substituted 3-30 membered, monocyclic, bicyclic or polycyclic ring having, in addition to the nitrogen, 0-10 heteroatoms;

L^{s} is L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
683. The method of Embodiment 682, wherein the N is bonded to H.
684. The method of Embodiment 682 or 683, wherein L^{s} is —C(R^{5s})₂, wherein each R^{5s} is independently R^{s}.
685. The method of any one of Embodiments 682-683, wherein L^{s} is optionally substituted —CH₂—.
686. The method of any one of Embodiments 682-685, wherein L^{s} is —CH₂—.
687. The method of any one of Embodiments 682-686, wherein

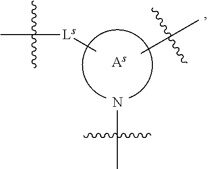

is optionally substituted

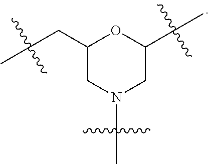 [SMILES: *CC1CN(*)CC(*)O1]

688. The method of any one of Embodiments 682-687, wherein

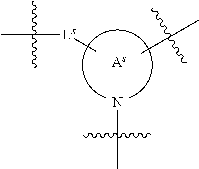

is optionally substituted

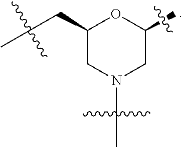

689. The method of any one of Embodiments 682-688, wherein

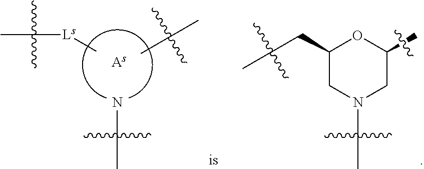

690. The method of Embodiment 681, wherein SU is

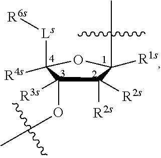

wherein:

each of R^{1s}, R^{2s}, R^{3s}, R^{4s}, R^{5s}, and R^{6s} is independently R^{s};

each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;

L^{s} is L;

each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};

each -Cy- is independently an optionally substituted bivalent 3-30 membered, monocyclic, bicyclic or polycyclic ring having 0-10 heteroatoms;

each Cy^{L} is independently an optionally substituted trivalent or tetravalent, 3-30 membered, monocyclic, bicyclic or polycyclic ring having 0-10 heteroatoms;

each R′ is independently —R, —C(O)R, —C(O)OR, or —S(O)₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
691. The method of Embodiment 690, wherein Cl is bonded to BA.
692. The method of any one of Embodiments 690-691, wherein L^{s} is —C(R^{5s})₂, wherein each R^{5s} is independently R^{s}.
693. The method of any one of Embodiments 690-692, wherein L^{s} is optionally substituted —CH₂—.
694. The method of any one of Embodiments 690-693, wherein L^{s} is —CH₂—.
695. The method of any one of Embodiments 690-694, wherein R^{1s} is —H.
696. The method of any one of Embodiments 690-695, wherein R^{3s} is —H.
697. The method of any one of Embodiments 690-696, wherein SU is

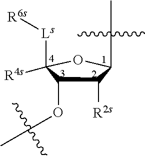

698. The method of any one of Embodiments 690-697, wherein R^{2s} is —H.
699. The method of any one of Embodiments 690-697, wherein R^{2s} is —F.
700. The method of any one of Embodiments 690-697, wherein R^{2s} is —OR, wherein R is C₁₋₆ aliphatic.
701. The method of any one of Embodiments 690-697, wherein R^{2s} is —OMe.
702. The method of any one of Embodiments 690-697, wherein R^{2s} is -MOE.
703. The method of any one of Embodiments 690-702, wherein R^{4s} is —H.
704. The method of any one of Embodiments 690-702, wherein R^{2s} and R^{4s} are taken together to form-L-.
705. The method of any one of Embodiments 690-702, wherein R^{2s} and R^{4s} are taken together to form-L-, wherein L is 2′-O—CH₂-4′, wherein the —CH₂— is optionally substituted.
706. The method of Embodiment 681, wherein SU is -L^{PS}—SU′- R^{6s}, wherein SU′ is an acyclic sugar.
707. The method of Embodiment 706, wherein L^{PS} is —O—.
708. The method of Embodiment 706, wherein L^{PS} is
709. The method of Embodiment 706, wherein L^{PS} is a covalent bond.
710. The method of any one of Embodiments 682-709, wherein R^{6s} is —O-L-R′.
711. The method of any one of Embodiments 682-710, wherein R^{6s} is —OH protected for oligonucleotide synthesis.
712. The method of any one of Embodiments 682-712, wherein R^{6s} is DMTrO—.
713. The oligonucleotide, compound, composition or method of any one of Embodiments 1-712, wherein an acyclic sugar has the structure of a′-L^{SA1}-L^{SA2}(-L^{SA3}-)-L^{SA4}-b′, wherein each of L^{SA1},L^{SA3}, and L^{SA4} is independently optionally substituted bivalent C₁₋₄ aliphatic or C₁₋₄ aliphatic having 1-3 heteroatoms, and L^{SA2} is optionally substituted CH or N.
714. The oligonucleotide, compound, composition or method of any one of Embodiments 1-712, wherein an acyclic sugar has the structure of a′-CH₂—CH(-L^{SA3}-)-CH₂-b′, wherein each of the CH₂ and CH is independently optionally substituted, and -L^{SA3}- is bonded to a nucleobase, and is —O—CH₂—, wherein the —CH₂— is optionally substituted.
715. The oligonucleotide, compound, composition or method of any one of Embodiments 1-712, wherein an acyclic sugar has the structure of a′-CH₂—CH(—O—CH₂—)—CH₂-b′, wherein each of the CH₂ and CH is independently optionally substituted.
716. The oligonucleotide, compound, composition or method of any one of Embodiments 1-712, wherein an acyclic sugar has the structure of a′-CH₂—CH(—O—CH₂—)—CH(CH₃)-b′, wherein each of the CH₂ and CH is independently optionally substituted.
717. The oligonucleotide, compound, composition or method of any one of Embodiments 1-712, wherein an acyclic sugar has the structure of a′-CH₂—CH(—O—CH(CH₃)—)—CH₂-b′, wherein each of the CH₂ and CH is independently optionally substituted.
718. The oligonucleotide, compound, composition or method of any one of Embodiments 1-712, wherein an acyclic sugar has the structure of a′-CH₂—CH(—O—CH(CH₂OH)—)—CH₂-b′, wherein each of the CH₂ and CH is independently optionally substituted.
719. The oligonucleotide, compound, composition or method of any one of Embodiments 1-712, wherein an acyclic sugar has the structure of a′-CH₂—CH(-L^{SA3}—)—CH₂-b′, wherein each of the CH₂ and CH is independently optionally substituted.
720. The oligonucleotide, compound, composition or method of any one of Embodiments 1-712, wherein an acyclic sugar has the structure of a′-CH₂—CH(O—CH₂—)—CH₂—NHR′-b′, wherein each of the CH₂ and CH is independently optionally substituted.
721. The oligonucleotide, compound, composition or method of any one of Embodiments 1-712, wherein an acyclic sugar has the structure of a′-CH₂—CH(O—CH₂—)—CH₂—N(CH₃)-b′, wherein each of the CH₂ and CH is independently optionally substituted.
722. The oligonucleotide, compound, composition or method of any one of Embodiments 1-712, wherein an acyclic sugar has the structure of a′-CH₂—CH(O—CH(CH₃)—)—CH₂—N(CH₃)-b′, wherein each of the CH₂ and CH is independently optionally substituted.
723. The oligonucleotide, compound, composition or method of any one of Embodiments 1-712, wherein an acyclic sugar has the structure of a′-CH₂—CH(O—CH(CH₂OH)—)—CH₂-b′, wherein each of the CH₂ and CH is independently optionally substituted.
724. The oligonucleotide, compound, composition or method of any one of the preceding Embodiments, wherein each heteroatom is independently boron, oxygen, sulfur, nitrogen, phosphorus, or silicon.
725. The oligonucleotide, compound, composition or method of any one of the preceding Embodiments, wherein each heteroatom is independently oxygen, sulfur, nitrogen, phosphorus, or silicon.
726. The oligonucleotide, compound, composition or method of any one of the preceding Embodiments, wherein each ring heteroatom is independently oxygen, sulfur, or nitrogen.
727. A method for modulating expression, level and or activity of a nucleic acid or a product thereof, comprising contacting the nucleic acid with an oligonucleotide or composition of any one of the any one of the preceding Embodiments.
728. A method for modulating expression, level and or activity of a nucleic acid or a product thereof in a system, comprising administering to the system an oligonucleotide or composition of any one of the any one of the preceding Embodiments.
729. The method of any one of Embodiments 727-728, wherein the expression, level and or activity of a nucleic acid or a product thereof is reduced.
730. A method for modulating splicing of a nucleic acid, comprising contacting the nucleic acid with an oligonucleotide or composition of any one of the any one of the preceding Embodiments.
731. A method for modulating splicing of a nucleic acid in a system, comprising administering to the system an oligonucleotide or composition of any one of the any one of the preceding Embodiments.
732. The method of any one of Embodiments 730-731, wherein skipping of a target exon is increased.
733. The method of any one of Embodiments 730-731, wherein inclusion of a target exon is increased.
734. The method of any one of Embodiments 727-733, wherein the nucleic acid is a transcript.
735. The method of any one of Embodiments 727-734, wherein base sequence of the oligonucleotide or oligonucleotides in the composition is complementary or identical to the base sequence of the nucleic acid.
736. The method of any one of Embodiments 727-735, wherein a system is an in vitro assay.
737. The method of any one of Embodiments 727-735, wherein a system is a cell.
738. The method of any one of Embodiments 727-735, wherein a system is a tissue.
739. The method of any one of Embodiments 727-735, wherein a system is an organ.
740. The method of any one of Embodiments 727-735, wherein a system is an organism.
741. The method of any one of Embodiments 727-735, wherein a system is an animal.
742. The method of any one of Embodiments 727-735, wherein a system is a subject.
743. The method of any one of Embodiments 727-735, wherein a system is a human.

### EXEMPLIFICATION

Certain examples of provided technologies (compounds (oligonucleotides, reagents, etc.), compositions, methods (methods of preparation, use, assessment, etc.), etc.) were presented herein.

### Example 1. Development of Useful Solid Support for Oligonucleotide Production.

Drying and Silylation of Native 600 A CPG: To a dried 1 L three RB flask was added Native 600 A CPG (50 g), Toluene (500 mL, 10 mL per g of CPG), and Dean—Stark apparatus was set up. The flask was fastened with overhead stirrer and while gently stirring the solution was heated at 148° C. for 4 h, then silylation reagent (Linkers 1-8, 146 μmol/g) was added at refluxing temperature, and the reaction continued for another 4 h. Then Silylation reagent (Linkers 1-8, 146 umol/g) was added again at refluxing temperature, and the reaction continued for another 4 h. The flask was cooled to rt under Argon. CPG washed sequentially with ACN (1 L), DCM (2 L), ACN (1 L and diethyl ether (500 mL) and CPG was transferred into a 1 L flask and dried under vacuum overnight.

3′—Succinate Nucleoside Loading: To a dried 1 L RB flask, was added 5′-ODMTr-2′-X (X═H, F, OCH3, Methoxyethyl-O-)-3′-triethylammonium-succinate-Nucleoside (2.81 g, 1.0 eq.), HBTU (3.6 g, 2.5 eq.), and then CH3CN (500 mL, 10 mL per g of CPG). To this solution, Et3N (2.6 mL, 5.0 eq.) and step 1 derivatized 600A CPG (50 g) was added and the flask was fastened on the mechanical twist shaker overnight. CPG washed with sequentially with ACN (1 L), DCM (2 L) and ACN (1 L) and CPG was transferred into a 1 L RB flask and dried under vacuum overnight.

Capping: Pyridine (400 mL) and acetic anhydride (100 mL) were added into a 5′-ODMTr-3′—Succinyl-LCAA-600A-CPG-2′-X (X═H, F, OCH₃, Methoxyethyl) Nucleoside containing flask and the flask was fastened on the mechanical twist shaker for 1 h. CPG washed with sequentially with ACN (1 L), DCM (2 L) and ACN (1 L) and diethyl ether (500 mL) and CPG was transferred into a 1 L RB flask and dried under vacuum overnight to give corresponding 5′-ODMTr-3′-Succinyl-LCAA-600A-CPG-2′-X (X═H, F, OCH3, Methoxyethyl-O-) Nucleoside.

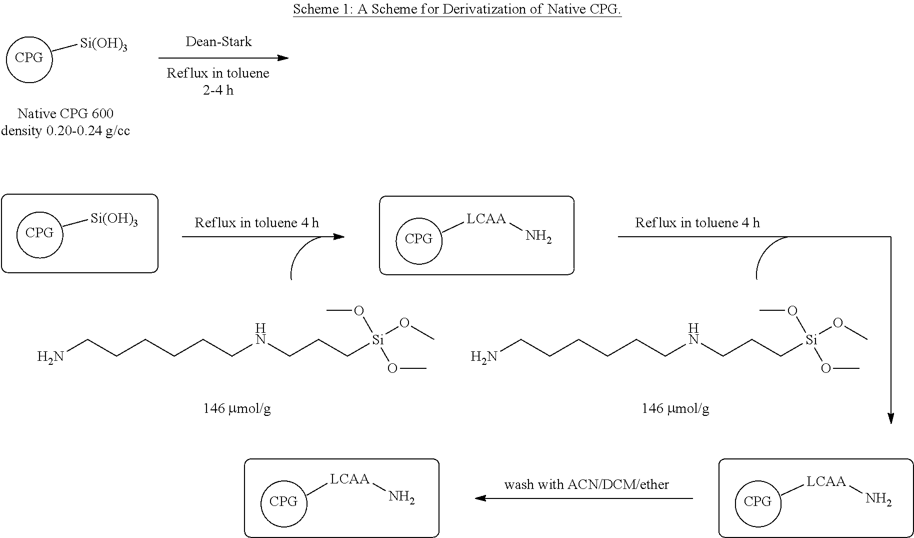

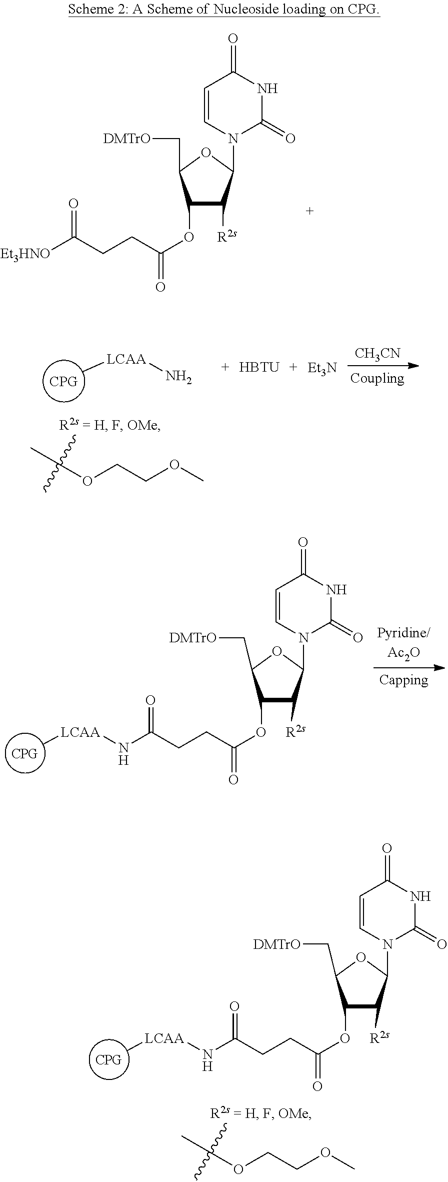

TABLE 1
Certain solid supports.

| | | | Targeted | |
|---|---|---|---|---|
| S. | | Loading | Loading | Amount |
| No | Solid Support | (μmol/g) | (μmol/g) | (g) |
| 1 | 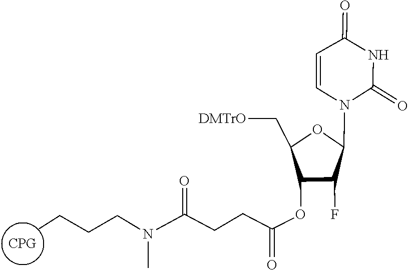 | 63 | 75 | 30 g |
| 2 | 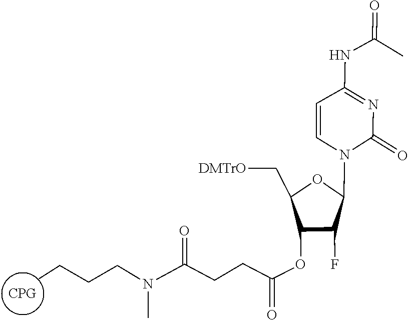 | 78 | 75 | 50 g |
| 3 | 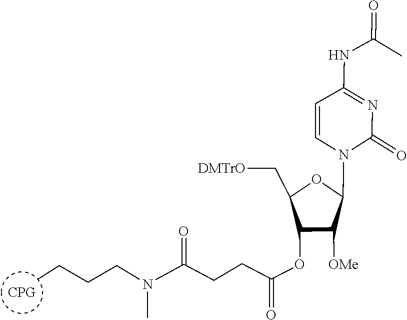 | 78 | 75 | 25 g |
| 4 | 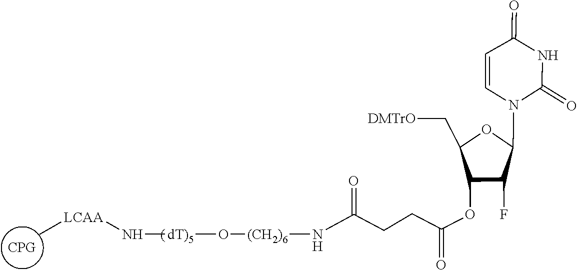 | 30 | 70 | 16 g |
| 5 | 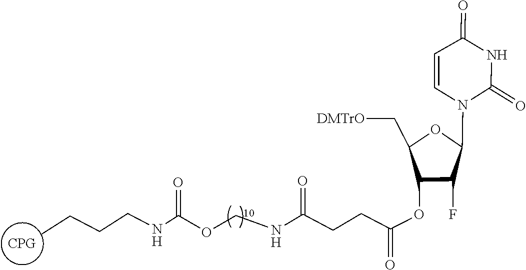 | 74 | 75 | 25 g |
| 6 | 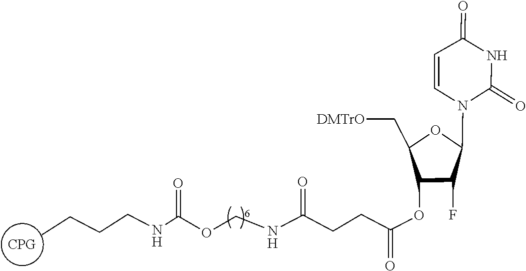 | 72 | 75 | 15 g |
| 7 | 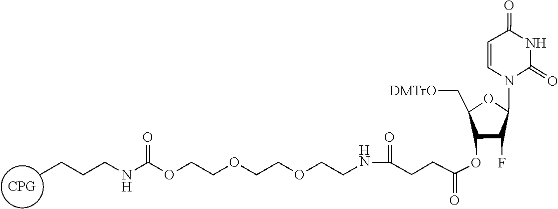 | 61 | 75 | 15 g |
| 8 | 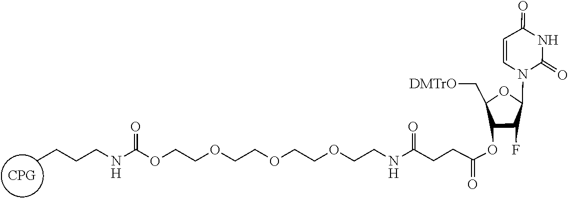 | 60 | 75 | 15 g |
| 9 | 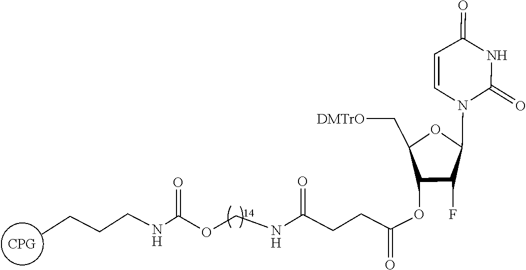 | 60 | 75 | 15 g |
| 10 | 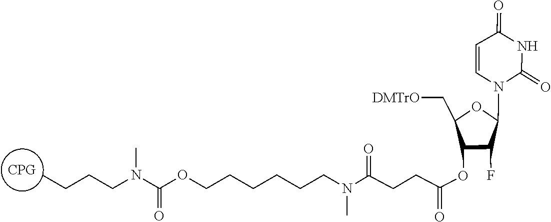 | 30 | 75 | 15 g |

Among other things, provided solid support technologies are particularly useful for preparing oligonucleotides and compositions herein. In some embodiments, provided linker moieties provide improved stability during synthesis yet can be efficiently cleaved when desired, and can provide significantly improved yields and/or purities compared to other linkers (e.g., those having comparable structure but no methyl on nitrogen).

Additional useful solid support for oligonucleotide product were developed.

Succinyl piperidine linker (SP-linker)

Loading piperidine linker: To a dried 100 mL RB flask was added amino-linker solid support (10 g), 1-(tert-butoxycarbonyl)-4-piperidinecarboxylic acid (688 mg, 3 mmol), PyNTP (4.5 g, 9 mmol) and dissolved in DCM (50 mL) and added DIPEA (2.6 mL, 15 mmol). The flask was fastened with overhead stirrer and gently stirring the solution at rt for 3.5 h. Solid support was washed sequentially with DCM (200 mL), pyridine (200 mL), and Et₂O (100 mL). Solid support was transferred into a 100 mL flask. Pyridine (10 mL) and acetic anhydride (10 mL), NMI (10 mL), and MeCN (20 mL) were added and the flask was fastened on the mechanical twist shaker for 1.5 h. Solid support was washed with sequentially with MeCN (200 mL), and Et₂O (100 mL). Solid support was transferred into a 100 mL flask. 0.1M TsOH in MeCN (500 mL) was added to the flask, and the flask was fastened on the mechanical twist shaker for 16 h. Solid support was washed with sequentially with MeCN (200 mL), 10% TEA in MeCN (200 mL), MeCN (200 mL), and Et₂O (100 mL) and solid support was transferred into a 500 mL flask and dried under vacuum overnight.

3′-Succinate Nucleoside Loading: To a dried 15 mL test tube, 5′-ODMTr-2′-X (X═H, OMe, LNA)-3′-triethylammonium-succinate-Nucleoside (160 umol), PyNTP (240 mg, 480 umol) was added, and dissolved in MeCN (5 mL). To this solution, DIPEA (140 uL, 800 umol) and piperidine linker derivatized solid support (500 mg) was added and the flask was fastened on the mechanical twist shaker overnight. Solid support was washed with sequentially with DCM (20 mL), pyridine (20 mL) and Et₂O (20 mL) and solid support was transferred into a 100 mL flask and dried under vacuum overnight.

Capping: Pyridine (1 mL) and acetic anhydride (1 mL), NMI (1 mL), and MeCN (2 mL) were added into a 5′-O-DMTr-3′-succinyl-piperidine-solidsupport-2′-X (X═H, OMe, LNA) Nucleoside containing flask and the flask was fastened on the mechanical twist shaker for 1.5 h. Solid support was washed with sequentially with MeCN (20 mL), and Et₂O (10 mL). Solid support was transferred into a 100 mL flask and dried under vacuum overnight to give corresponding 5′-O-DMTr-3′-succinyl-piperidine-solidsupport-2′-X (X═H, OMe, LNA) Nucleoside.

Certain solid supports.

| | | Loading | Amount |
|---|---|---|---|
| S. No | Solid Support | (μmol/g) | (g) |
| 11 | 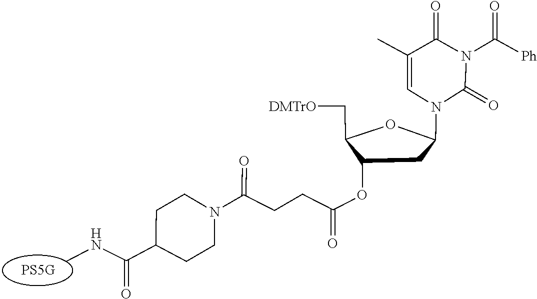 | 83 | 1 g |
| 12 | 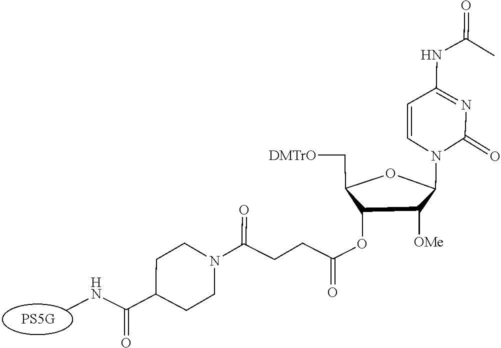 | 84 | 3 g |
| 13 | 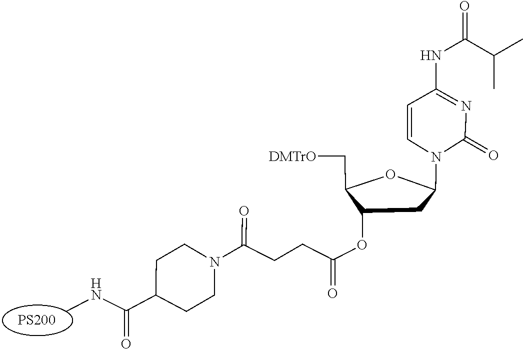 | 86 | 1 g |
| 14 | 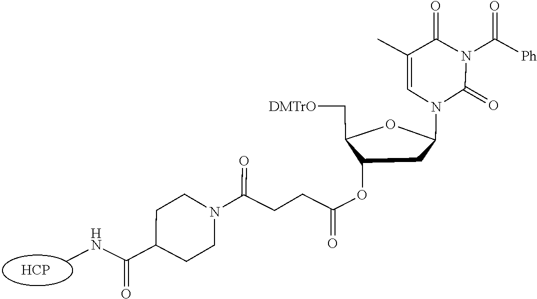 | 57 | 1 g |
| 15 | 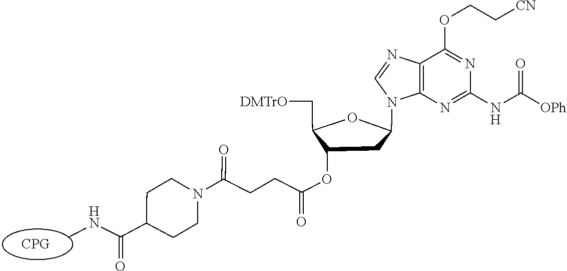 | 85 | 500 mg |
| 16 | 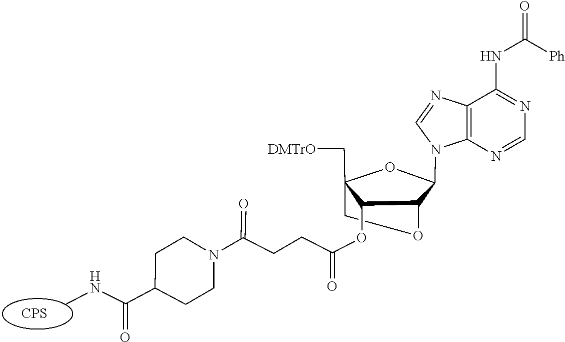 | 129 | 1 g |

Imido linker (IM-linker)

Loading imido-linker: To a dried 20 mL test tube, amino-linker solid support (1 g), trimellitic anhydride (192 mg l mmol) and pyridine (5 mL) were added. The flask was fastened with overhead stirrer and gently stirring the solution at rt for 18 h. Solid support was washed sequentially with DCM (25 mL) and Et₂O (25 mL). Solid support was transferred into a 50 mL flask and dried under vacuum overnight.

3′-OH Nucleoside Loading: To a dried 20 mL test tube, 5′-ODMTr-2′-X (X═H, LNA)-3′-OH-Nucleoside (200 umol), PyNTP (300 mg, 600 umol) was added, and dissolved in DCM (5 mL). To this solution, TEA (139 uL, 1 mmol) and imido-linker derivatized solid support (1 g) was added and the flask was fastened on the mechanical twist shaker overnight. Solid support was washed with sequentially with DCM (20 mL), pyridine (20 mL) and Et₂O (20 mL) and solid support was transferred into a 100 mL flask and dried under vacuum overnight.

Capping: Pyridine (5.4 mL) and acetic anhydride (0.6 mL) were added into a 5′-O-DMTr-3′-imido-linker-solidsupport-2′-X (X═H, LNA) Nucleoside containing flask and the flask was fastened on the mechanical twist shaker for 3 h. Solid support was washed with sequentially with MeCN (20 mL), and Et₂O (10 mL). Solid support was transferred into a 100 mL flask and dried under vacuum overnight to give corresponding 5′-O-DMTr-3′-imido-linker-solidsupport-2′-X (X═H, LNA) Nucleoside.

Certain cnliel cunnnrtc

| | | Loading | Amount |
|---|---|---|---|
| S. No | Solid Support | (μmol/g) | (g) |
| 17 | 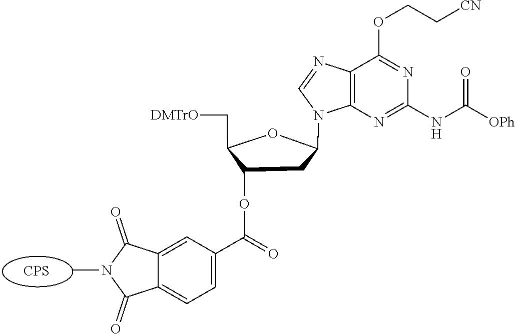 | 89 | 1 g |
| 18 | 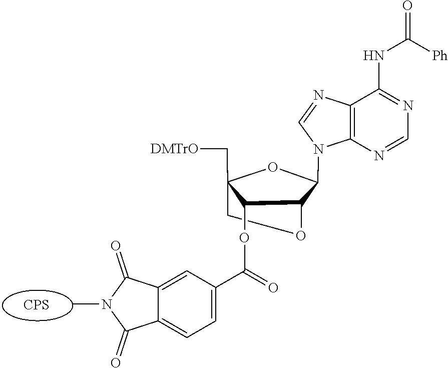 | 41 | 1 g |
| 19 | 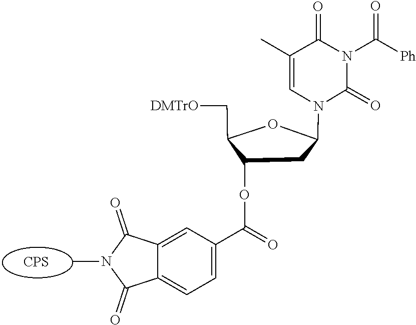 | 180 | l g |

To assess new linkers, WV-14118 was synthesized using DPSE chiral amidites. WV-14118: fA*SfU*SfU*SfU*SfC*SfU
Base sequence: AUUUCU
Linkage/Stereochemistry: SSSSS

In this example, preparation of WV-14118 was conducted on various solid supports, using 2.7×2.1 cm stainless steel column at a scale of around 200 μmol.

Abbreviation:

| Abbreviation | Name of Raw Materials |
|---|---|
| fU-CPG | 2′-Fluoro-2′-deoxy-U via linker CPG (600 Å LBD), |
| | described in table XX |
| fA-L-DPSE | 5′-O-DMT-fA (Bz)-(L)-DPSE Phosphoramidite |
| fC-L-DPSE | 5′-O-DMT-fC (Ac)-(L)-DPSE Phosphoramidite |
| fU-L-DPSE | 5′-O-DMT-fU-(L)-DPSE Phosphoramidite |
| fU-CEP | 5′-O-DMT-fU-β-Cyanoethyl Phosphoramidite |
| Cap A | N-Methylimidazole in Acetonitrile, 20/80, v/v |
| Cap B | Acetic Ahydride/2,6-Lutidine/Acetonitrile, 20/30/50, v/v/v |
| CE | β-Cyanoethyl |
| CEP | β-Cyanoethyl Phosphoramidite |
| Conc. NH₄OH | 28-30% Concentrated Ammonium Hydroxide |
| CT | Contact Time |
| CV | Column Volume |
| CMIMT | 1-(Cyanomethyl)-1H-imidazol-3-ium-1-yl |
| | Trifluoromethanesulfonate |
| DCA | Dichloroacetic Acid |
| Deblock | 3% DCA in Toluene |
| DMSO | Dimethylsulfoxide |
| ETT | 5-Ethylthio-1H-tetrazole |
| Filter unit | Filter Device, 0.22 micron filter, CORNING |
| IBN | Isobutyronitrile |
| MeCN | Acetonitrile |
| MS3A | Molecular Sieves, 3 Å |
| NLT | No less than |
| NMI | N-methylimidazole |
| Ox | 0.04-0.06M Iodine in pyridine/water, 90/10, v/v |
| TEA | Triethylamine |
| TEA.3HF | Triethylamine Trihydrofluoride |
| XH | Xanthane Hydride |
| WFI | Water for Injection (water) |

Certain solid supports.

| | | Loading |
|---|---|---|
| S. No | Solid Support | (μmol/g) |
| 1 | 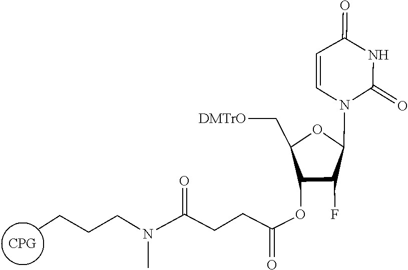 | 89 |
| 5 | 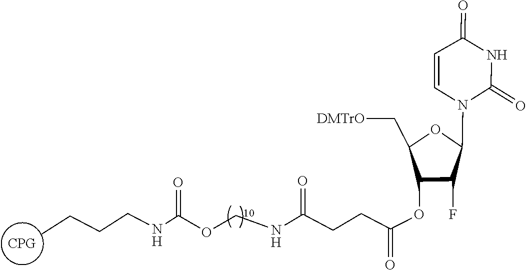 | 74 |
| 6 | 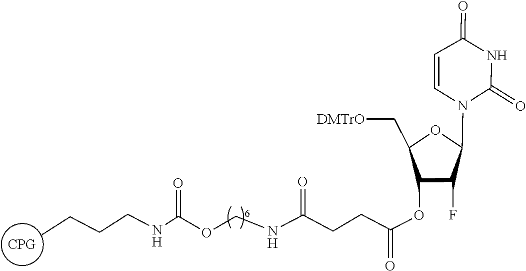 | 72 |
| 7 | 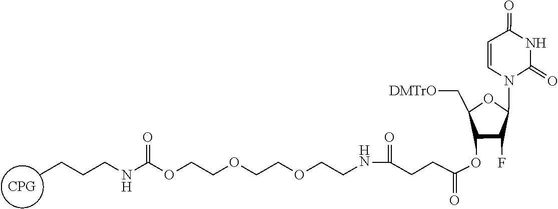 | 61 |
| 8 | 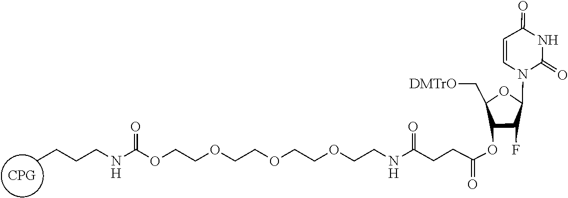 | 60 |
| 9 | 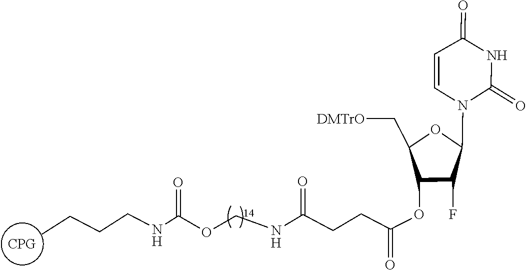 | 60 |
| 20 | 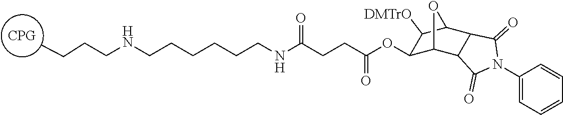 | 76 |
| 21 | 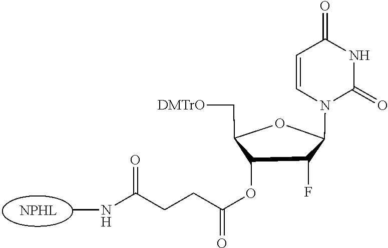 | 205 |

Example Synthesis Process Parameters.

| Process Step | Parameter | Set Points |
|---|---|---|
| Synthesis | DPSE Chiral Amidite | 1. Detritylation |
| Cycle | | 2. Coupling-1 |
| | | 3. Cap-1 |
| | | 4. Modification - Thiolation |
| | | 5. Cap-2 |
| | Standard Amidite | 1. Detritylation |
| | | 2. Coupling-2 |
| | | 3. Modification - Oxidation |
| | | 4. Cap-2 |
| Column, Scale, | Column | Stainless Steel |
| Synthesizer | Column Diameter | 2.7 cm |
| | Column Bed Height | 2.1 cm |
| | Column Volume | 12.0 mL |
| | Scale | about 200 μmol |
| | Synthesizer | AKTA OligoPilot 100 |
| CPG | Solid Support | fU-CPG |
| | Support Loading | 70~80 μmol/g |
| | Adjusted Support Density | 0.24 g/c.c. |
| Pre-Synthesis | Pre-Synthesis Wash Solvent | MeCN |
| Wash | Pre-Synthesis Wash Flow Rate | 424 cm/h |
| | Pre-Synthesis Wash Volume | 2.0 CV |
| De-blocking | Reagent | Deblock |
| (Detritylation) | Control Mode | UV watch command |
| | Detrit Wavelength | 436 nm |
| | Detrit UV Watch | On: 500 mAu with 1.2 CV delay |
| | | Off: 1000 mAu |
| | UV Autozero | Autozero on Deblock in bypass |
| | | prior to the 1^{st} detrit |
| | Detrit Flow Rate | 424 cm/h |
| | Post Detritylation MeCN Wash | 4.0 CV |
| | | 424 cm/h |
| Coupling-1 | Amidite Eq./Support | 2.5 eq. |
| | Concentration and Diluent of | 0.2M of fA-L-DPSE, fC-L-DPSE, |
| | Amidite | fU-L-DPSE in IBN-MeCN (20:80), and |
| | | fC-L-DPSE, fU-CEP in 100% MeCN |
| | Density of the Amidite | Non-adjusted |
| | Solution | |
| | Drying of Amidite Solution | MS3A, 15-20% w.r.t. Amidite solution, v/v |
| | | Drying Time = NLT 4 h |
| | Activator | CMIMT |
| | Activator Concentration | 0.5M in MeCN |
| | Activator Eq./Support | 10.20 |
| | Density of Activator Solution | Non-adjusted |
| | Drying of Activator Solution | MS3A, 10%, w.r.t. Activator solution, v/v |
| | | Drying Time = NLT 4 h |
| | Activator/Amidite Molar Ratio | 5.833:1 |
| | Activator vol % | 70% |
| | Coupling Charge Flow Rate | 30.5 cm/h |
| | for all Amidites | |
| | Coupling Charge Flow Rate | 71.1 cm/h |
| | for Activator | |
| | Coupling Charge Volume | 8.1 mL |
| | (Amidite + Activator) | |
| | Push Volume | 4.5 mL |
| | Recycle Flow Rate | 212 cm/h |
| | Recycle Times | 10 min |
| | MeCN Wash Flow Rate | 424 cm/h |
| | MeCN Wash Volume | 2.0 CV |
| Coupling-2 | Amidite Eq./Support | 2.5 eq. |
| | Concentration and Diluent of | 0.2M of fU-CEP in 100% MeCN |
| | Amidite | |
| | Density of the Amidite | Non-adjusted |
| | Solution | |
| | Drying of Amidite Solution | MS3A, 15-20% w.r.t. Amidite solution, v/v |
| | | Drying Time = NLT 4 h |
| | Activator | ETT |
| | Activator Concentration | 0.5M in MeCN |
| | Activator Eq./Support | 10.31 |
| | Density of Activator Solution | Non-adjusted |
| | Drying of Activator Solution | MS3A, 10%, w.r.t. Activator solution, v/v |
| | | Drying Time = NLT 4 h |
| | Activator/Amidite Molar Ratio | 3.75:1 |
| | Activator Vol % | 60% |
| | Coupling Charge Flow Rate | 32.3 cm/h |
| | for all Amidites | |
| | Coupling Charge Flow Rate | 48.5 cm/h |
| | for Activator | |
| | Coupling Charge Volume | 6.5 mL |
| | (Amidite + Activator) | |
| | Push Volume | 4.5 mL |
| | Recycle Flow Rate | 212 cm/h |
| | Recycle Times | 8 min |
| | Coupling MeCN Wash Flow | 424 cm/h |
| | Rate | |
| | Coupling MeCN Wash Volume | 2.0 CV |
| Cap 1 | Mode | Flow Through |
| | Reagent | Cap B |
| | Column Volume (CV) | 2.0 CV |
| | Contact Time (CT) | 4 min |
| | Reagent Charge Flow Rate | 6.0 mL/min |
| | MeCNPush Volume | 1.3 CV |
| | MeCNPush Flow Rate | 6.0 mL/min |
| Modification | Mode | Flow Through |
| (Thiolation) | Reagent | 0.1M XH in pyridine-MeCN (1:1, v/v) |
| | Density of thiolation reagent | Non-adjusted |
| | Charge Volume | 1.2 CV |
| | Charge Flow Rate | 26.4 cm/h |
| | Contact Time | 6 min |
| | MeCNPush Flow Rate | 26.4 cm/h |
| | MeCNPush Volume | 1.3 CV |
| | Post-thio MeCN Wash Flow | 424 cm/h |
| | Rate (Flow_AB) | |
| | Post-thio MeCN Wash Volume | 1.0 CV |
| Modification | Mode | Flow Through |
| (Oxidation) | Reagent | Ox |
| | Iodine Eq./Solid Support | 3.5 eq. |
| | Contact Time | 2.0 min |
| | Reagent Charge Flow Rate | 75.5 cm/h |
| | MeCNPush Flow Rate | 75.5 cm/h |
| | MeCNPush Volume | 1.3 CV |
| | MeCN Wash Flow rate | 424 cm/h |
| | MeCN Wash Volume | 1.0 CV |
| Cap 2 | Reagent | Cap A |
| | | Cap B |
| | Mode | Flow Through |
| | Charge Volume | 0.4 CV |
| | Contact Time | 0.8 min |
| | Charge Flow Rate | Cap A Pump A = 31.5 cm/h |
| | | Cap B Pump B = 31.5 cm/h |
| | MeCN Push Volume_AB | 1.3 CV |
| | MeCN Push Flow Rate_AB | MeCN Pump A = 31.5 cm/h |
| | | MeCN Pump B = 31.5 cm/h |
| | Capping Wash Volume AB | 1.0 CV |
| | Capping Wash Flow Rate AB | 424 cm/h |
| Post Synthesis | MeCN Wash Flow Rate | 424 cm/h |
| MeCN Wash | MeCN Wash Flow Volume | 2.0 CV |

Cleavage and deprotection (C&D) was conducted at a scale of 200 μmol. Certain useful cleavage & deprotection process parameters are presented below:

Example recipe for preparation of a DS1 solution (1 L)

| Solvent/Reagents | Required Volume (mL) |
|---|---|
| DMSO | 733 ± 36.7 |
| WFI | 147 ± 7.4 |
| TEA | 70 ± 3.5 |
| TEA.3HF | 50 ± 2.5 |

Example WV-14118 cleavage and deprotection parameters

| Process Step | Parameter | Set Points | |
|---|---|---|---|
| Removal of | Reaction Vessel | Appropriate for TEA-HF/ | |
| DPSE group | | Ammonium Hydroxide | |
| | | solution | |
| | Scale | 746 | μmol |
| | Reagent | DSI | |
| | Volume | 100 ± 5 | mL/mmol |
| | Reaction Temp | 28 ± 2.5 | ° C. |
| | Incubator shaker rpm | 180 ± 10 | rpm |
| | Reaction Time | 1.5 ± 0.5 | hrs |
| Removal of | Reaction Vessel | Appropriate for TEA-HF/ | |
| Residual | | Ammonium Hydroxide | |
| Protecting | | solution | |
| group | Reagent | Conc. NH₄OH | |
| | Reagent Volume | 200 ± 5 | mL/mmol |
| | Reaction Time | 3 ± 1 | hrs |
| | Reaction Temperature | 37 ± 2 | ° C. |
| | Cooling to room temp | <25 | ° C. |
| | prior to filtration | | |
| Filtration | Initial Crude | Filter unit | |
| | Filtration Device | | |
| | Filtration Mode | Under vacuum | |
| | Support Wash Solvent | WFI | |
| | Support Wash Volume | 250-350 | mL/mmol |

SQD analysis of crude 6-mer summarized below.

Crude analysis of WV-14118 using various linkers.

| S. | | Percentage of each peak, % | | | | | | crude |
|---|---|---|---|---|---|---|---|---|
| No | Loading | 1-mer | 2-mer | 3-mer | 4-mer | 5-mer | 6-mer | OD/umol |
| 1 | 89 μmol/g | 8.68 | 5.22 | 5.05 | 3.03 | 5.26 | 72.77 | 26.67 |
| 5 | 74 μmol/g | 2.25 | 1.97 | 6.34 | 4.32 | 8.67 | 76.45 | 36.53 |
| 6 | 72 μmol/g | 3.42 | 3.35 | 6.63 | 4.15 | 6.78 | 75.67 | 34.52 |
| 7 | 61 μmol/g | 4.27 | 1.70 | 3.94 | 3.35 | 5.90 | 80.85 | 31.41 |
| 8 | 60 μmol/g | 6.86 | 3.81 | 6.43 | 4.49 | 7.61 | 70.80 | 31.50 |
| 9 | 60 μmol/g | 2.92 | 1.66 | 4.89 | 5.26 | 11.09 | 74.17 | 31.49 |
| 20 | 76 μmol/g | 8.97 | 2.61 | 3.49 | 2.33 | 6.17 | 76.43 | 26.48 |
| 21 | 205 μmol/g | 0.39 | 0.44 | 1.60 | 3.31 | 14.11 | 80.15 | 31.17 |

### Example 2. Coupling Partners for Oligonucleotide Synthesis

Useful experimental procedure (A) for chloro reagents (Compounds 504-506)

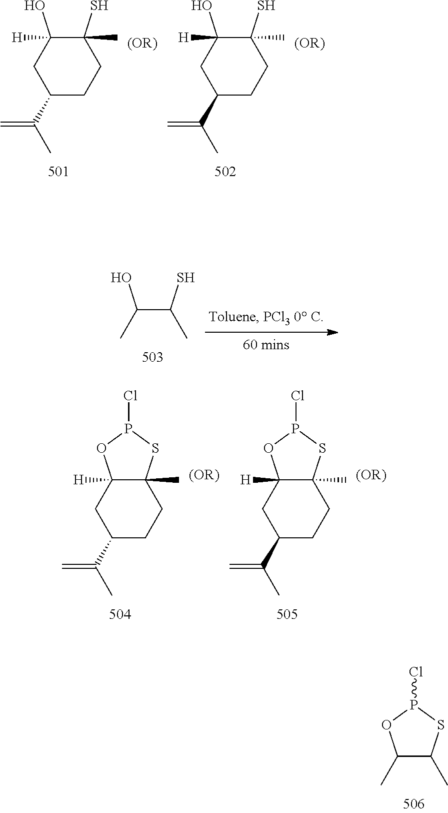

Thiol (82.12 mmol) was dissolved in toluene (100 mL) under argon (250 mL single neck flask) then 4-methylmorpholine (18.0 mL, 164.24 mmol) was added. This mixture was added dropwise via cannula over 30 min to an ice-cold solution of phosphorus trichloride (7.2 mL, 82.12 mmol) in toluene (100 mL) under argon atmosphere. After warming to room temperature for 1 h, the mixture was filtered carefully under vacuum/argon. The resulting filtrate was concentrated by rotary evaporation (flushing with Ar) then dried under high vacuum for 4 h. The resulting crude compound was isolated as thick oil, which was dissolved in THF to obtain a 1 M stock solution and this solution was used in the next step without further purification.

Compound 504: Synthesized from compound 501. ³¹P NMR (162 MHz, THF-CDCl₃, 1:2) δ 207.89.

Compound 505: Synthesized from compound 502. ³¹P NMR (162 MHz, THF-CDCl₃, 1:2) δ 6 207.89.

Compound 506: Synthesized from compound 503. ³¹P NMR (162 MHz, THF-CDCl₃, 1:2) δ 205.92, 205.67, 205.53.

Useful experimental procedure (B) for monomers (Compounds 511-514)

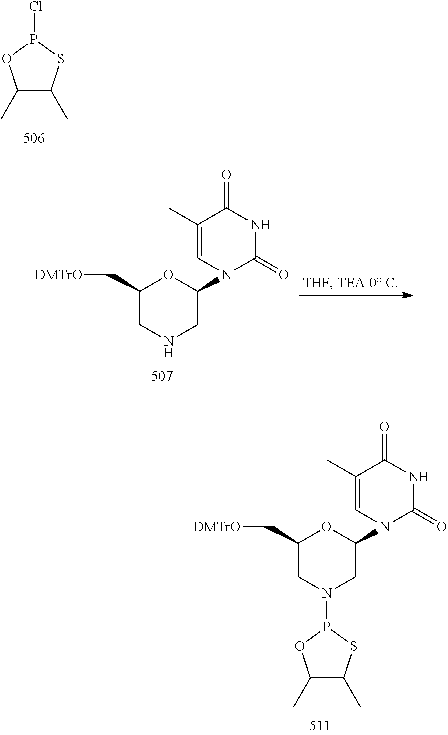

The 5′-ODMTr protected morpholino nucleoside (45.9 mmol) was dried in a three neck 250 mL round bottom flask by co-evaporating with anhydrous toluene (100 mL) followed by under high vacuum for 18 h. The dried nucleoside was dissolved in dry THF (150 mL) under argon atmosphere. Then, triethylamine (170.2 mmol, 3.7 equiv.) was added into the reaction mixture, then cooled to ˜−10° C. A THF solution of the crude chloro reagent (1 M solution, 1.6 equiv., 73.5 mmol) was added to the above mixture through cannula over ˜5 min, then, gradually warmed to room temperature over about 1 h. LCMS showed that the starting material was consumed. The reaction mixture was filtered carefully under vacuum/argon and the resulting filtrate was concentrated under reduced pressure to give a yellow foam which was further dried under high vacuum overnight. Crude mixture was purified by silica gel column [Column was pre-deactivated using acetonitrile then ethyl acetate (5% TEA) and then equilibrated using ethyl acetate-hexanes] chromatography using ethyl acetate and hexane as eluents.

Structure of certain protected morpholino nucleosides:

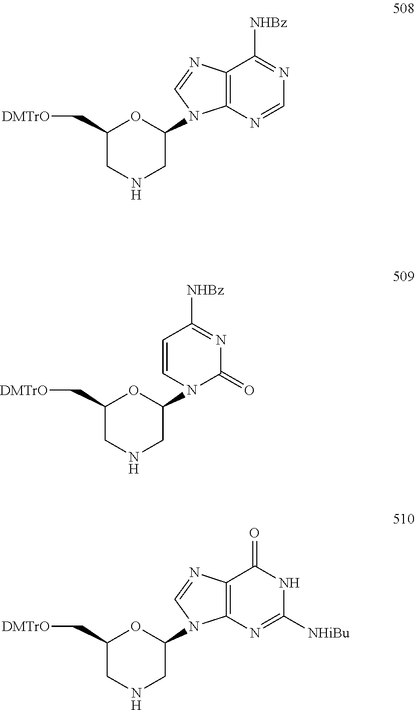

Structure of certain morpholino monomers:

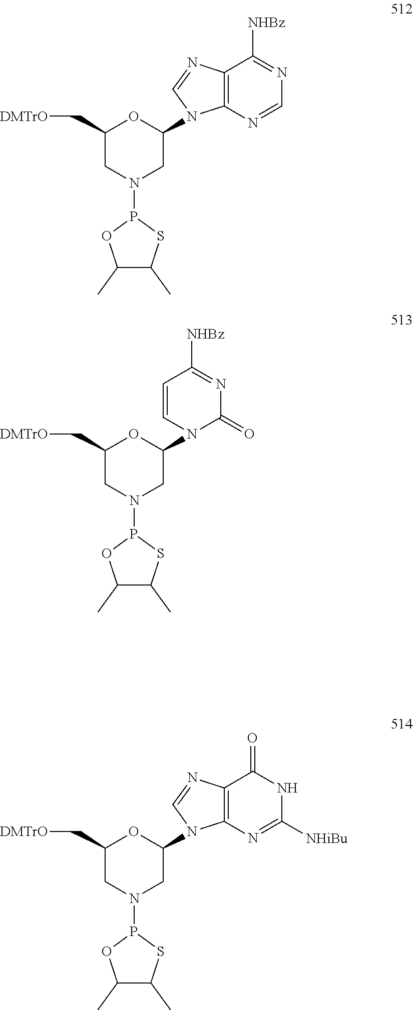

Compound 511: Yield 82%. Reaction was carried out using 506 and 507 by following procedure B. ³¹P NMR (202 MHz, CDCl₃) δ 158.56, 158.28, 153.27, 152.28, 143.23, 141.61, 138.59, 137.11; MS (ES) m/z calculated for C₃₅H₄₀N₂O₇PS [M+Na]⁺700.22, Observed: 700.63 [M+Na]⁺.

Compound 512: Yield 61%. Reaction was carried out using 506 and 508 by following procedure B. ³¹P NMR (162 MHz, CDCl₃) δ 158.59, 158.38, 153.11, 152.94, 143.39, 142.59, 138.10, 137.80; MS (ES) m/z calculated for C₄₂H₄₃N₆O₆PS [M+H]⁺791.27, Observed: 791.42 [M+H]⁺.

Compound 513: Yield 81%. Reaction was carried out using 506 and 509 by following procedure B. ³¹P NMR (162 MHz, CDCl₃) δ 158.75, 158.67, 153.14, 151.90, 144.47, 141.45, 139.07, 136.52; MS (ES) m/z calculated for C₄₁H₄₃N₄O₇PS [M+H]⁺767.26, Observed: 767.63 [M+H]⁺.

Compound 514: Yield 81%. Reaction was carried out using 506 and 510 by following procedure B. ³¹P NMR (202 MHz, CDCl₃) δ 158.25, 157.89, 152.75, 152.72, 143.67, 141.94, 137.81, 137.62; MS (ES) m/z calculated for C₃₉H₄₅N₆O₇PS [M+14]⁺773.28, Observed: 773.70 [M+H]⁺.

Structure of certain stereopure morpholino monomers:

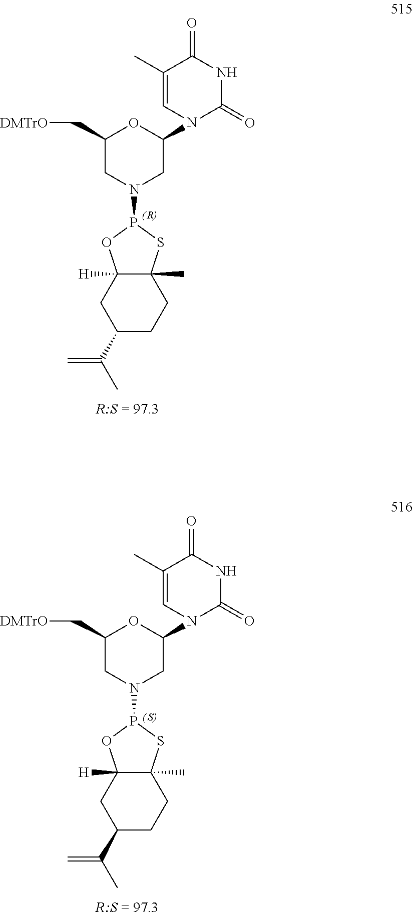

Compound 515: Yield 48%. Reaction was carried out using 501 and 507 by following procedure B. ³¹P NMR (162 MHz, CDCl₃) (R:S=97:3); δ 156.27 (S), 138.87 (R); MS (ES) m/z calculated for C₄₁H₄₈N₃O₇PS [M+Na]⁺780.87, Observed: 780.33 [M+Na]⁺.

Compound 516: Yield 62%. Reaction was carried out using 502 and 507 by following procedure B. ³¹P NMR (162 MHz, CDCl₃) (R:S=3:97); δ 155.47 (R), 137.60 (S); MS (ES) m/z calculated for C₄₁H₄₈N₃O₇PS [M+Na]⁺780.87, Observed: 780.24 [M+Na]⁺.

Useful experimental procedure (C) for morpholino P-N dimers (Compounds 518-520):

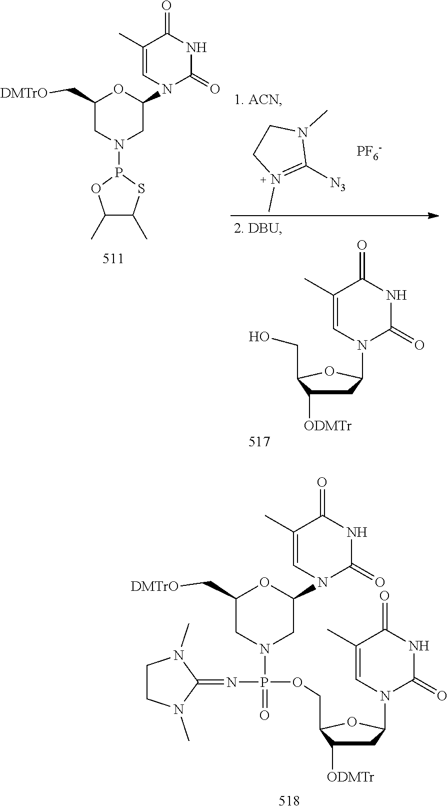

To a stirred solution of morpholino monomer (0.27 mmol, 2 equiv., pre-dried by co-evaporation with dry acetonitrile and kept it under vacuum for minimum 12 h) in dry acetonitrile (1.3 mL) was added a solution of 2-azido-1,3-dimethylimidazolinium hexafluorophosphate (0.34 mmol, 2.5 equiv.) in acetonitrile (0.4 mL) under argon atmosphere at room temperature. Resulting reaction mixture was stirred for 10 mins then DMTr protected alcohol (0.14 mmol, pre-dried by co-evaporation with dry acetonitrile and kept it under vacuum for minimum 12 h) in dry acetonitrile (1 mL) and 1,8-Diazabicyclo [5.4.0] undec-7-ene (0.68 mmol, 5 equ, 0.68 ml of 1 M solution in dry acetonitrile) are added. Once the reaction was completed (monitored by LCMS) then the reaction mixture was concentrated under reduced pressure then re-dissolved in DCM, washed with aq. NaHCO3 then the organic layer was evaporated to give the crude product. The crude mixture was purified by silica gel column using dichloromethane and methanol as eluents.

Structure of certain stereopure morpholino PN-dimers:

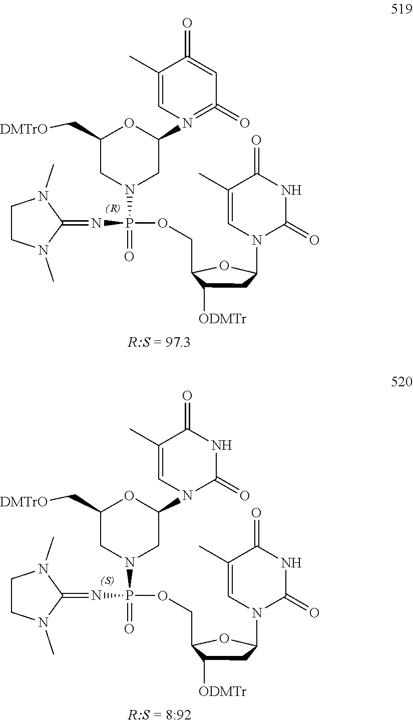

Compound 518 (stereorandom): Yield 82%. Reaction was carried out using 511 and 517 by following procedure C. ³¹P NMR (162 MHz, CDCl₃) δ 4.13, 3.99; MS (ES) m/z calculated for C₆₇H₇₃N₈O₁₄P [M+Na]⁺1267.48, Observed: 1267.91 [M+Na]⁺.

Compound 519 (stereopure (Rp)): Yield 93%. Reaction was carried out using 516 and 517 by following procedure C. ³¹P NMR (162 MHz, CDCl₃) (R:S=96:4) δ 4.28 (R), 3.95 (S) ; MS (ES) m/z calculated for C₆₇H₇₃N₈O₁₄P [M+Na]⁺1267.48, Observed: 1267.34 [M+Na]⁺.

Compound 520 (stereopure (Sp)): Reaction was carried out using 515 and 517 by following procedure C. ³¹P NMR (162 MHz, CDCl₃) (R:S=8:92); δ 4.24 (R), 4.02 (S); MS (ES) m/z calculated for C₆₇H₇₃N₈O₁₄P [M+Na]⁺1267.48, Observed: 1267.91 [M+Na]⁺.

Useful experimental procedure (D) for morpholino P—S dimers (Compounds 521-523):

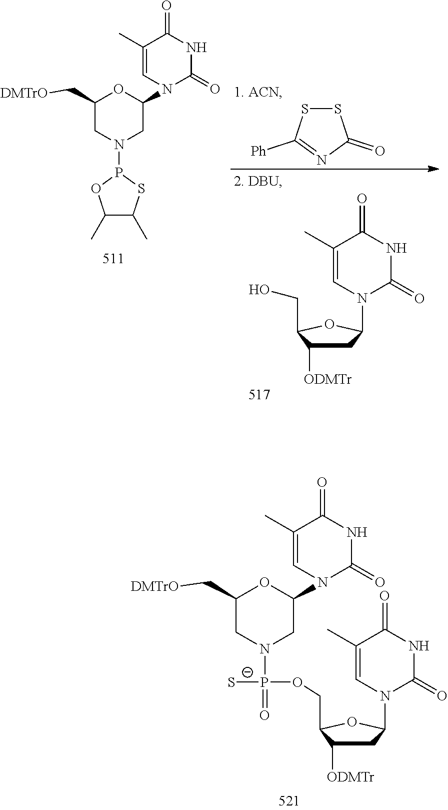

To a stirred solution of morpholino monomer (0.73 mmol, 1 equiv., pre-dried by co-evaporation with dry acetonitrile and kept it under vacuum for minimum 12 h) in dry acetonitrile (2 mL) was added a solution of 5-phenyl-3H-1,2,4-dithiazol-3-one (0.95 mmol, 1.3 equiv., 0.2 M) in acetonitrile under argon atmosphere at room temperature. Resulting reaction mixture was stirred for 10 min then DMTr protected alcohol (0.73 mmol, pre-dried by co-evaporation with dry acetonitrile and kept it under vacuum for minimum 12 h) in dry acetonitrile (2 mL) and 1,8-Diazabicyclo [5.4.0] undec-7-ene (7.3 mmol, 10 equ, 1 M solution in dry acetonitrile) are added. Once the reaction was completed (monitored by LCMS) then the reaction mixture was concentrated under reduced pressure then purified by silica gel column using dichloromethane and methanol as eluents.

Structure of certain stereopure morpholino PS-dimers:

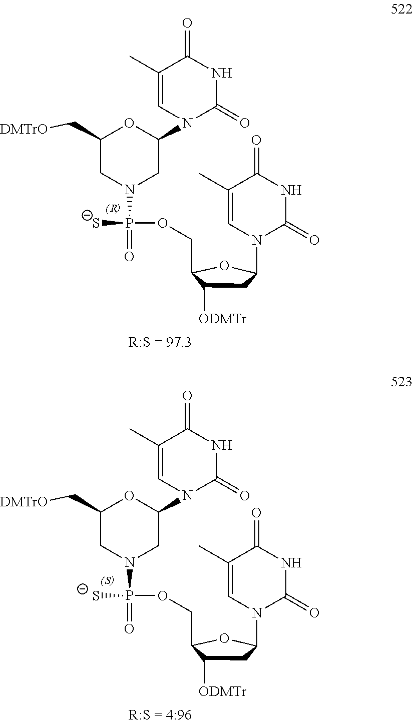

Compound 521 (stereorandom): Yield 62%. Reaction was carried out using 511 and 517 by following procedure D. ³¹P NMR (162 MHz, CDCl₃) δ 62.20, 61.01; MS (ES) m/z calculated for C₆₂H₆₃N₅O₁₄PS my 1164.38, Observed: 1164.49 my.

Compound 522 (stereopure (Rp)): Yield 56%. Reaction was carried out using 516 and 517 by following procedure D. ³¹P NMR (162 MHz, CDCl₃) (R:S=97:3) δ 62.23 (R), 61.03 (S); MS (ES) m/z calculated for C₆₂H₆₃N₅O₁₄PS [M]⁻1164.38, Observed: 1164.61 [M]⁻.

Compound 523 (stereopure (Sp)): Yield 52%. Reaction was carried out using 515 and 517 by following procedure D. ³¹P NMR (162 MHz, CDCl₃) (R:S=4:96) δ 62.20 (R), 61.01 (S); MS (ES) m/z calculated for C₆₂H₆₃N₅O₁₄PS [M]⁻1164.38, Observed: 1164.41 [M]⁻.

Assignment of Stereochemistry

The following procedures were utilized to assign the stereochemistry of the phosphorus center of stereopure monomers and dimers. Based on prior data, L-DPSE phosphoramidite using CMIMT for coupling, followed by modification, provided 4 with Rp. D-DPSE, first P-modified, then coupled under DBU condition, provided compound 4 with Rp. Compound 3, first P-modified, then coupled under DBU condition, provided compound 4 with Rp. It was inferred that compound 5, which has the same Rp configuration as compound 3, would provide compound 6 with Sp when first P-modified and then coupled under DBU condition.

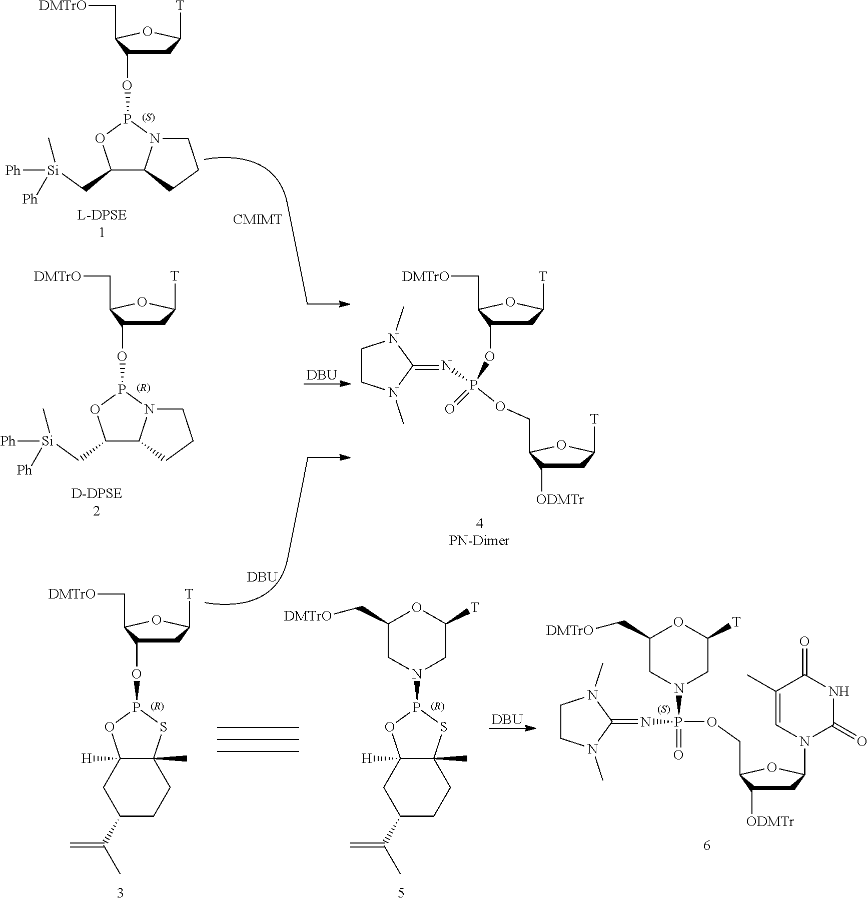

### Example 3. Preparation of Certain Oligonucleotides and Compositions

In an example, automated solid-phase synthesis of chirally controlled oligonucleotide compositions was performed according to cycles shown in Table 2A (regular amidite cycle, for PO linkages (natural phosphate linkages)), Table 2B (DPSE amidite cycle, for chirally controlled PS linkages (phosphorothioate internucleotidic linkages)), and Table 2C (MBO amidite cycle, for morpholino PN linkages) at 24 umol scale.

TABLE 2A
Regular Amidite Synthetic Cycle.

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | oxidation | 50 mM I₂/pyridine-H₂O (9:1, v/v) | 2.0 mL | 1 min |
| 4 | cap-2 | 20% Ac₂O, 30% 2,6-lutidine/MeCN | 1.0 mL | 45 s |
| | | 20% MeIm/MeCN | 1.0 mL | |

TABLE 2B
DPSE Amidite Synthetic Cycle.

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | cap-1 | 20% Ac₂O, 30% 2,6-lutidine/MeCN | 2.0 mL | 2 min |
| 4 | sulfurization | 0.2M XH/pyridine | 2.0 mL | 6 min |
| 5 | cap-2 | 20% Ac₂O, 30% 2,6-lutidine/MeCN | 1.0 mL | 45 s |
| | | 20% MeIm/MeCN | 1.0 mL | |

TABLE 2C
MBO Amidite Synthetic Cycle.

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 10 min |
| | | 1.0M DBU/MeCN | 1.0 mL | |
| 3 | cap-2 | 20% Ac₂O, 30% 2,6-lutidine/MeCN | 1.0 mL | 45 s |
| | | 20% MeIm/MeCN | 1.0 mL | |

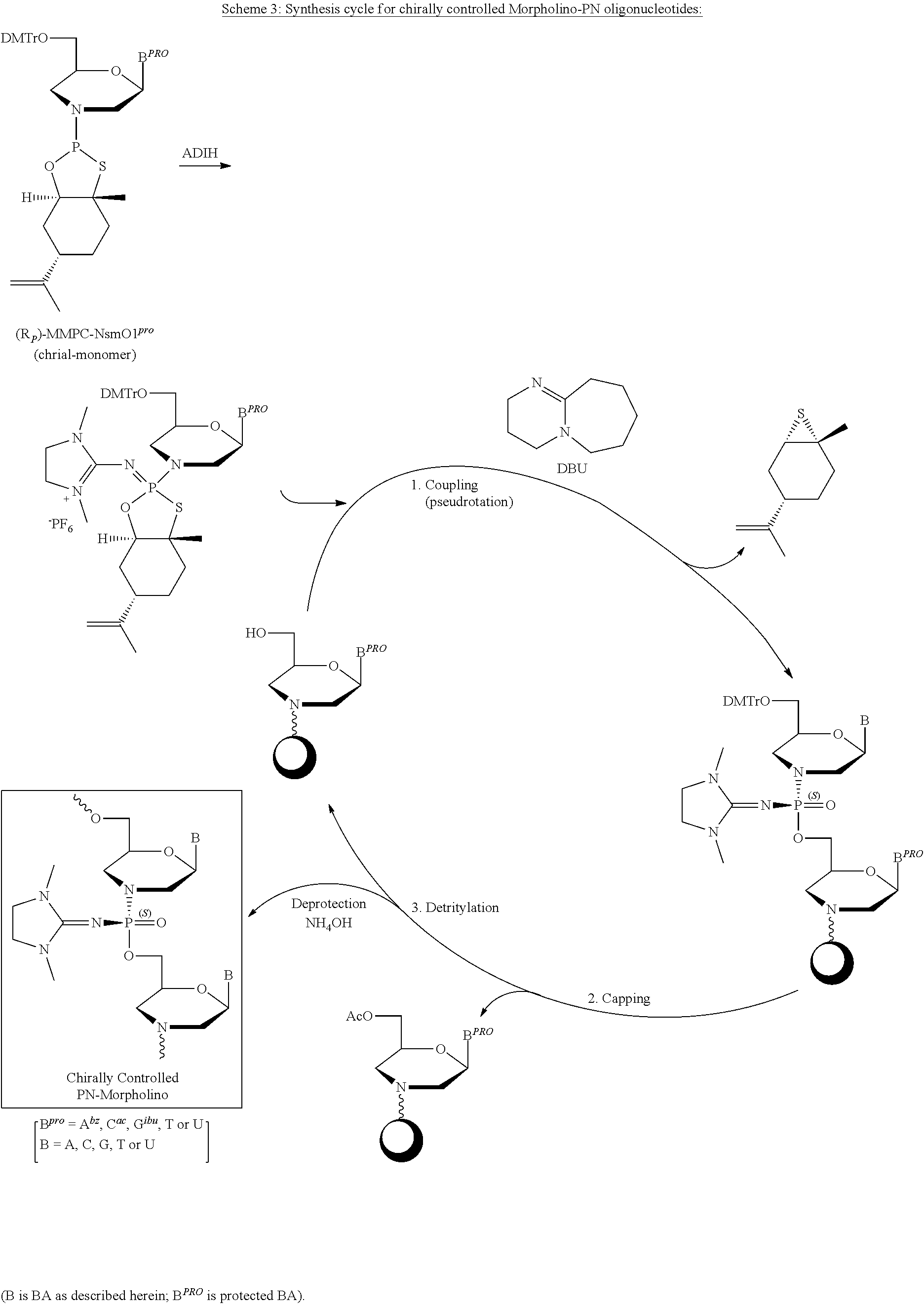

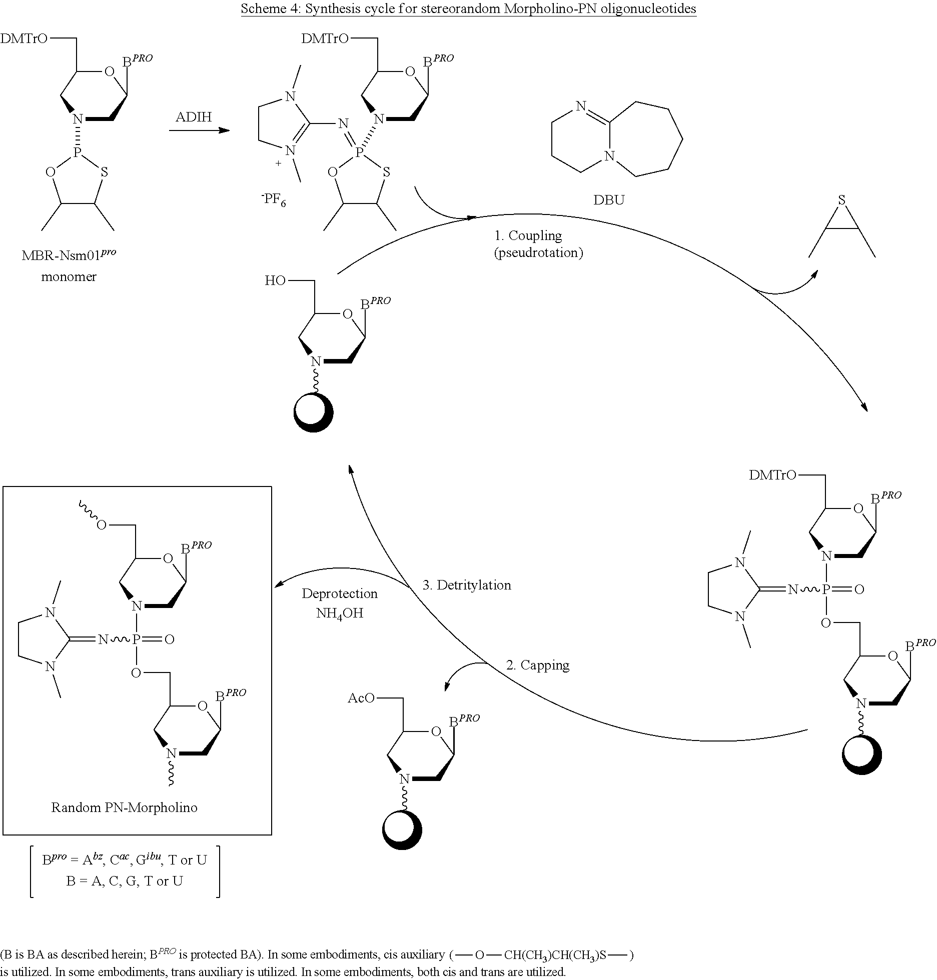

Useful procedure for the C&D conditions (24 μmol scale).

After completion of the synthesis, the CPG solid support was dried and transferred into 15 mL plastic tube. The CPG was treated with 1× reagent (2.4 mL; 100 uL/umol) for 6 h at 28° C., then added conc. NH₃ (aqueous solution, 4.8 mL; 200 umol/umol) for 24 h at 37° C. The reaction mixture was cooled to room temperature and the CPG was separated by membrane filtration, washed with 14 mL of H₂O. The crude material (filtrate) was analyzed by LTQ and RP-UPLC.

The crude materials were purified by AEX-HPLC with a linear gradient of 2.5M NaCl in 20 mM NaOH, desalting by tC₁₈ SepPak cartridge, and lyophilized to obtain the products. Results from certain preparations are presented below:

| | Crude | Crude | Final | Final | | |
|---|---|---|---|---|---|---|
| | UPLC | Mass | UPLC | Mass | calcd. | Observed |
| ID | Purity | Purity | Purity | Purity | Mass | Mass |
| WV-28299 | 55.44 | 88.18 | 94.82 | 97.56 | 6845.62 | 6846.0 |
| WV-28468 | 51.70 | 77.31 | 87.23 | 93.50 | 7003.85 | 7003.3 |
| WV-28469 | 54.80 | 75.97 | 90.47 | 86.98 | 6971.66 | 6971.7 |
| WV-28470 | 56.29 | 81.04 | 94.09 | 90.90 | 7257.10 | 7256.3 |
| WV-10258 | 50.01 | 71.74 | 96.79 | 94.81 | 6695.84 | 6694.9 |
| WV-11345 | 69.00 | 72.39 | 95.56 | 89.36 | 6981.28 | 6979.3 |
| WV-21218 | 52.52 | 85.75 | 90.87 | 77.76 | 6949.15 | 6946.7 |
| WV-28294 | 58.47 | 73.46 | 93.02 | 97.72 | 6892.22 | 6893.5 |
| WV-28471 | 48.11 | 72.98 | 93.66 | 97.11 | 6902.27 | 6903.2 |
| WV-28472 | 47.90 | 70.34 | 93.11 | 94.16 | 7070.50 | 7071.6 |
| WV-28475 | 31.10 | NA* | 80.92 | NA* | 7701.59 | 7701.9 |
| WV-28476 | 50.15 | 63.27 | 93.27 | 96.57 | 6938.25 | 6938.9 |

*Direct injection utilized for MS.

### Abbreviations:

1× reagent:

ADIH: 2-azido-1,3-dimethylimidazolium hexafluorophosphate

AEX-HPLC: anion exchange high pressure liquid chromatography

CMIMT: N-cyanomethylimidazolium triflate

CPG: controlled pore glass

DBU: 1,8-diazabicyclo[5.4.0]undec-7-ene

DCM: dichloromethane, CH₂Cl₂

DMTr: 4,4′-dimethoxytrityl

HF: hydrofluoride

IBN: isobutyronitrile

Melm: N-methylimidazole

TCA: trichloroacetic acid

TEA: triethylamine

XH: xanthane hydride

### Example 4. Preparation of Certain Oligonucleotides and Compositions

In an example, automated solid-phase synthesis of chirally controlled oligonucleotide compositions was performed according to the cycles shown in Table 3A (regular amidite cycle, for PO linkages (natural phosphate linkages)), Table 3B (DPSE amidite cycle, for chirally controlled PS linkages (phosphorothioate internucleotidic linkages)), and Table 3C (M-CBM amidite cycle, for morpholino carbamate linkages (—C(O)—O—, which is part of a carbamate group)).

TABLE 3A
Regular Amidite Synthetic Cycle.

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | oxidation | 50 mM I₂/pyridine-H₂O (9:1, v/v) | 2.0 mL | 1 min |
| 4 | cap-2 | 20% Ac₂O, 30% 2,6-lutidine/MeCN | 1.0 mL | 45 s |
| | | 20% MeIm/MeCN | 1.0 mL | |

TABLE 3B
DPSE Amidite Synthetic Cycle.

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | cap-1 | 20% Ac₂O, 30% 2,6-lutidine/MeCN | 2.0 mL | 2 min |
| 4 | sulfurization | 0.2M XH/pyridine | 2.0 mL | 6 min |
| 5 | cap-2 | 20% Ac₂O, 30% 2,6-lutidine/MeCN | 1.0 mL | 45 s |
| | | 20% MeIm/MeCN | 1.0 mL | |

TABLE 3C
M-CBM Amidite Synthetic Cycle.

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 10 min |
| | | 1.0M DBU/MeCN | 1.0 mL | |
| 3 | cap-2 | 20% Ac₂O, 30% 2,6-lutidine/MeCN | 1.0 mL | 45 s |
| | | 20% MeIm/MeCN | 1.0 mL | |

Useful procedure for the C&D conditions (24 μmol scale):

After completion of the synthesis, the CPG solid support was dried and transferred into 15 mL plastic tube. The CPG was treated with 1× reagent (2.4 mL; 100 uL/umol) for 6 h at 28° C., then added conc. NH₃ (aqueous solution, 4.8 mL; 200 umol/umol) for 24 h at 37° C. The reaction mixture was cooled to room temperature and the CPG was separated by membrane filtration, washed with 14 mL of H₂O. The crude material (filtrate) was analyzed by LTQ and RP-UPLC. The crude materials were purified by AEX-HPLC with a linear gradient of 2.5M NaCl in 20 mM NaOH, desalting by tC18 SepPak cartridge, and lyophilized to obtain the products. Certain results were presented in Table 3D.

TABLE 3D
Certain Preparations.

| entry | WV-ID | %-FLP, UPLC | LC-MS |
|---|---|---|---|
| 1 | WV-37972 | 47.02% | calculated 6730.4; observed 6730.1 |
| 2 | WV-37973 | 35.91% | calculated 66473; observed 6646.7 |
| 3 | WV-37976 | 39.64% | calculated 6730.4; observed 6729.9 |
| 4 | WV-37980 | 42.79% | calculated 6809.5; observed 6809.9 |
| 5 | WV-37981 | 44.74% | calculated 6904.6; observed 6904.1 |
| 6 | WV-37982 | 46.08% | calculated 6983.7; observed 6982.9 |

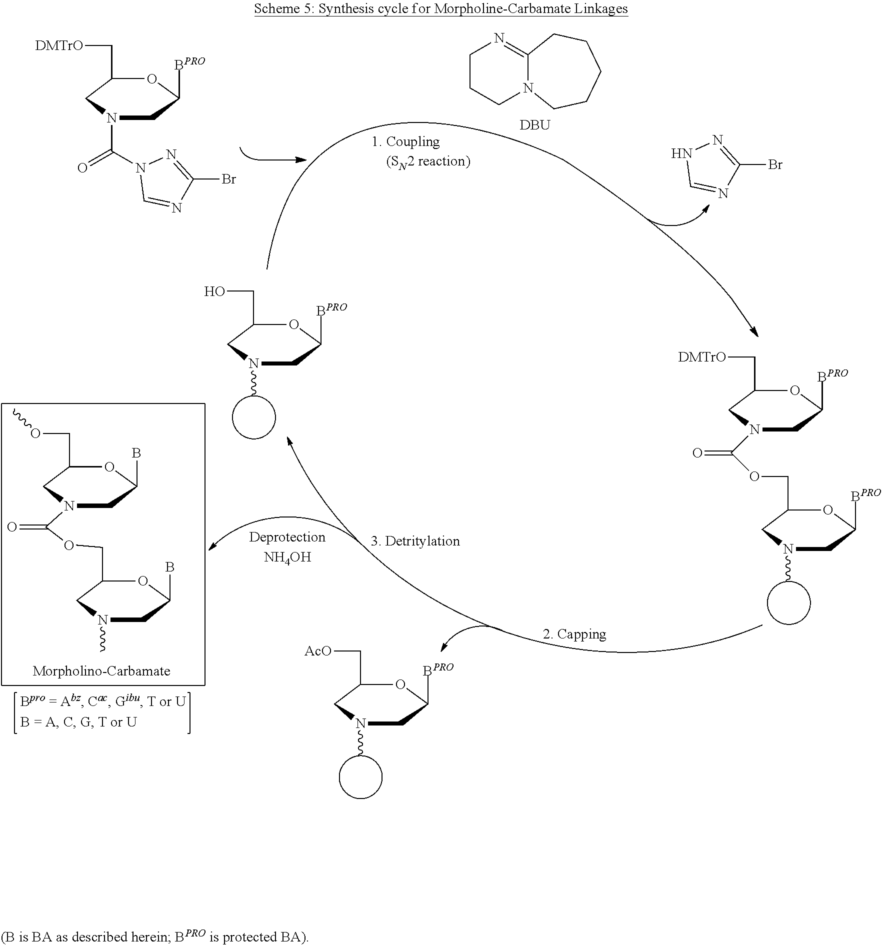

Various reagents useful for preparing oligonucleotides, e.g., those described in Tables A1, A2, A3, A4, etc. are described, e.g., in Examples below.

### Example 5. Synthesis of WV-NU-097

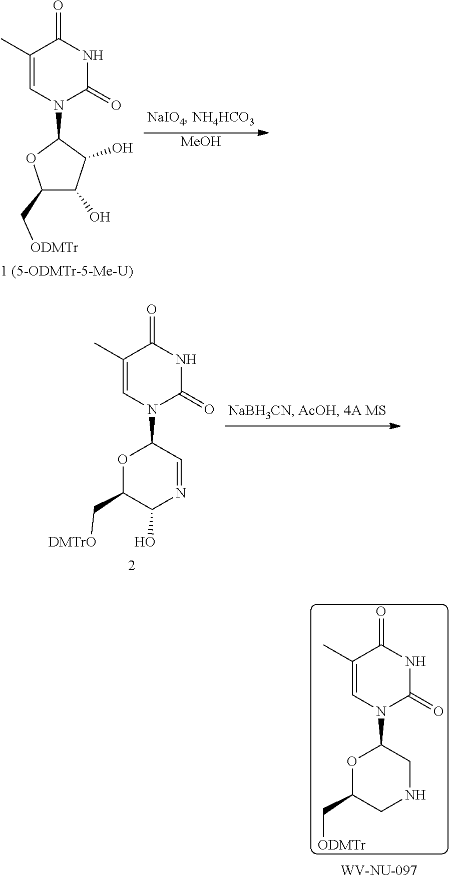

Compound 1 (5′-DMTr-T) (100 g, 178.38 mmol) was dissolved in MeOH (2 L), NaIO₄ (41.97 g, 196.22 mmol) and NH₄HCO₃ (28.20 g, 356.77 mmol) were added. The mixture was stirred at 25° C. for 3 hours. TLC indicated compound 1 was consumed completely and one new spot formed. The reaction mixture was filtered. Compound 2 (99.46 g, crude) was obtained in MeOH. TLC (Petroleum ether: Ethyl acetate=0: 1), Rf=0.69.

To the solution of compound 2 (99.46 g, 549.44 mmol) in MeOH was added NaBH₃CN (39.23 g, 624.31 mmol), 4 Å MS (32 g, 178.37 mmol) and AcOH (16.07 g, 267.56 mmol). The mixture was stirred at 15° C. for 17 hr. TLC showed compound 2 was consumed and a main new spot formed. The reaction mixture was filtered, and the filtrate from three identical batches were combined and concentrated. The residue was dissolved in 2-methyltetrahydrofuran (4 L) and washed with sat. NaHCO₃ aq. (2 L). The combined aqueous layers were back-extracted with 2-methyltetrahydrofuran (1 L). The combined organic layers were dried over anhydrous Na₂SO₄, filtered and concentrated to afford a crude yellow solid. The crude was purified by column chromatography on silica gel (Petroleum ether/Ethyl acetate=3/1 to 0/1, then ethyl acetate/methanol=20/1 to 10/1, 5% TEA) to give compound WV-NU-097 (193.4 g, 62.69% yield, 91.187% purity) as a white solid. ¹H NMR (400 MHz, CHLOROFORM-d) δ ppm 1.27 (t, J=7.32 Hz, 2H) 1.80-1.88 (m, 3H) 2.46-2.64 (m, 2H) 2.92-3.07 (m, 4H) 3.12-3.28 (m, 2H) 3.67 (d, J=1.00 Hz, 6H) 3.91-3.99 (m, 1H) 5.81-5.88 (m, 1H) 6.72 (d, J=8.88 Hz, 4H) 7.06-7.13 (m, 1H) 7.14-7.20 (m, 2H) 7.21-7.29 (m, 5H) 7.36 (d, J=7.38 Hz, 2H). ¹³C NMR (101 MHz, CHLOROFORM-d) δ=171.20, 164.63, 158.52, 151.40, 144.77, 135.93, 135.80, 135.59, 130.09, 130.07, 128.16, 127.79, 126.78, 113.13, 111.45, 85.99, 80.35, 64.61, 60.40, 55.23, 55.17, 49.11, 46.70, 46.36, 21.04, 14.20, 12.60, 8.74. LCMS (M−H⁺): 542.2; purity: 91.19%. TLC (Petroleum ether/Ethyl acetate=0:1), Rf=0.11.

### Example 6. Synthesis of WV-NU-099

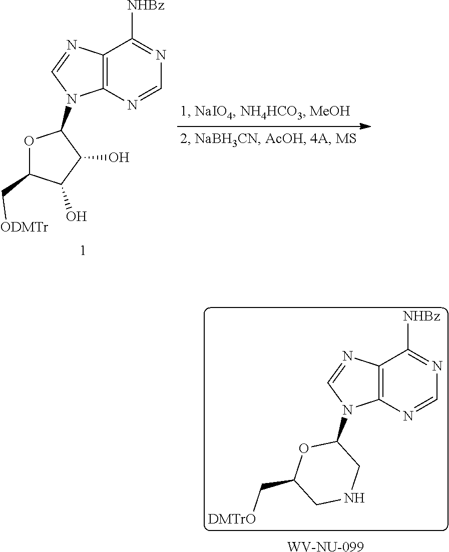

To a solution of compound 1 (100 g, 148.43 mmol) in MeOH (2000 mL) was added NaIO₄ (34.92 g, 163.27 mmol) and NH₄HCO₃ (23.47 g, 296.86 mmol). The mixture was stirred at 15° C. for 3 hr. 4 Å MS (60 g), NaBH₃CN (32.65 g, 519.51 mmol), and AcOH (13.37 g, 222.65 mmol) were added to the mixture. The mixture was stirred at 20° C. for 17 hr. TLC indicated compound 1 was consumed and one new spot formed. The reaction mixture was filtered, and the filtrate was combined and concentrated. The residue was dissolved in 2-methyltetrahydrofuran (3 L) and washed with sat. NaHCO₃ aq. (1.5 L) followed by brine (1.5 L). The combined organic layers were dried over anhydrous Na₂SO₄, filtered and concentrated to afford a crude yellow solid. The residue was purified by column chromatography (SiO₂, ethyl acetate/methanol=1/1, 5% TEA). The crude product WV-NU-099 (54 g) was obtained as a yellow solid. The crude was purified by column chromatography (SiO₂, Ethyl acetate/Methanol=1/0 to 10/1). Compound WV-NU-099 (37.5 g, 90.025% purity) was obtained as a white solid. ¹H NMR (400 MHz, CHLOROFORM-d) δ=9.07-8.77 (m, 1H), 8.59 (s, 1H), 8.03 (s, 1H), 7.79 (d, J=7.4 Hz, 2H), 7.42-7.35 (m, 1H), 7.33-7.26 (m, 2H), 7.22 (d, J=7.5 Hz, 2H), 7.14-7.03 (m, 6H), 7.02-6.96 (m, 1H), 6.60 (d, J=8.3 Hz, 4H), 5.77 (dd, J=2.4, 10.1 Hz, 1H), 3.89 (s, 1H), 3.66-3.51 (m, 6H), 3.19 (dd, J=2.1, 12.1 Hz, 1H), 3.10 (dd, J=5.1, 9.6 Hz, 1H), 2.99-2.88 (m, 3H), 2.66-2.52 (m, 1H). ¹³C NMR (101 MHz, CHLOROFORM-d) δ=171.21, 158.52, 152.63, 151.22, 149.54, 144.66, 140.85, 135.86, 135.74, 133.53, 132.86, 130.28, 130.07, 130.03, 128.84, 128.11, 127.94, 127.84, 126.86, 122.82, 113.63, 113.13, 86.10, 80.82, 77.73, 64.28, 60.42, 55.24, 50.52, 47.40, 46.47, 21.08, 14.21, 8.65. LCMS: NEG (M−H⁺)=655.3; purity, 90.02%. TLC: (Petroleum ether: Ethyl acetate=0:1) Rf=0.12.

### Example 7. Synthesis of WV-NU-100

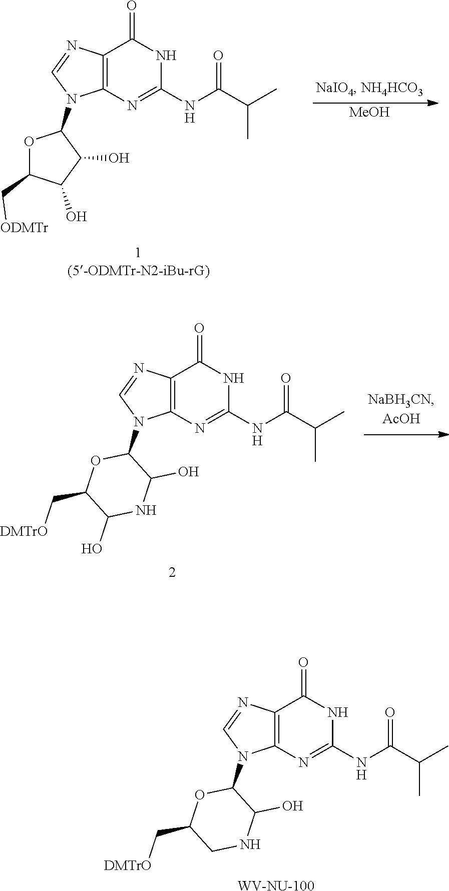

Compound 1 (5′-DMTr-T) (100 g, 152.51 mmol) was dissolved in MeOH (1.5 L), NaIO₄ (39.14 g, 183.01 mmol) and NH₄HCO₃ (30.14 g, 381.27 mmol) were added. The mixture was stirred at 20° C. for 5 hours. TLC indicated complete consumption of starting material. The resulting mixture was filtered. Compound 2 (102 g, crude) was obtained in MeOH.

To the solution of compound 2 (102 g, 152.08 mmol) in MeOH was added NaBH₃CN (33.45 g, 532.27 mmol), 4A MS (60 g, 152.08 mmol) and AcOH (13.70 g, 228.12 mmol). Stirring was continued at 20° C. for 18 hr. TLC indicated compound 2 was consumed completely and many new spots formed. The reaction mixture was filtered, and the filtrate from four identical batches were combined and concentrated. The residue was dissolved in 2-methyltetrahydrofuran (3 L) and washed with sat. NaHCO3 aq. (2 L). The combined organic layers were dried over anhydrous Na₂SO₄, filtered and concentrated to afford a crude yellow solid. The residue was purified by silica gel chromatography (Ethyl acetate/MeOH=100/1 to 9/1, 5% TEA) to give compound WV-NU-100 (70 g) and 190 g crude needed further purification. The 190 g crude was purified by MPLC (SiO₂, Ethyl acetate/MeOH=1/0 to 8/1, 5% TEA) to give compound WV-NU-100 (40 g) as a white solid. Obtained WV-NU-100 (110 g, 31.43% yield, 88.21% purity) total and 100 g crude in hand. ¹H NMR (400 MHz, CHLOROFORM-d) δ ppm 1.15-1.26 (m, 9H) 2.54-2.67 (m, 1H) 2.67-2.91 (m, 2H) 3.06-3.24 (m, 5H) 3.30 (br d, J=11.26 Hz, 1H) 3.73 (s, 6H) 4.22 (br s, 1H) 5.95 (br d, J=8.50 Hz, 1H) 6.77 (br d, J=8.63 Hz, 4H) 7.09-7.32 (m, 8H) 7.40 (br d, J=7.50 Hz, 2H) 7.86 (s, 1H). ¹³C NMR (101 MHz, CHLOROFORM-d) δ ppm 8.84 (s, 1 C) 14.18 (s, 1 C) 18.93 (s, 1 C) 19.08 (s, 1 C) 21.01 (s, 1 C) 36.15 (s, 1 C) 46.67 (s, 1 C) 49.98 (s, 1 C) 55.23 (s, 1 C) 60.37 (s, 1 C) 64.43 (s, 1 C) 80.48 (s, 1 C) 86.05 (s, 1 C) 113.14 (s, 1 C) 113.66 (s, 1 C) 120.43 (s, 1 C) 126.82 (s, 1 C) 127.79 (s, 1 C) 128.13 (s, 1 C) 130.08 (s, 1 C) 130.26 (s, 1 C) 135.85 (s, 1 C) 136.83 (s, 1 C) 144.77 (s, 1 C) 148.03 (s, 1 C) 148.22 (s, 1 C) 158.54 (s, 1 C) 171.12 (s, 1 C). LCMS (M−H⁺): 637.4; purity: 88.21%. TLC (Ethyl acetate/Methanol=5:1), Rf=0.14.

### Example 8. Synthesis of WV-NU-099-imidazole

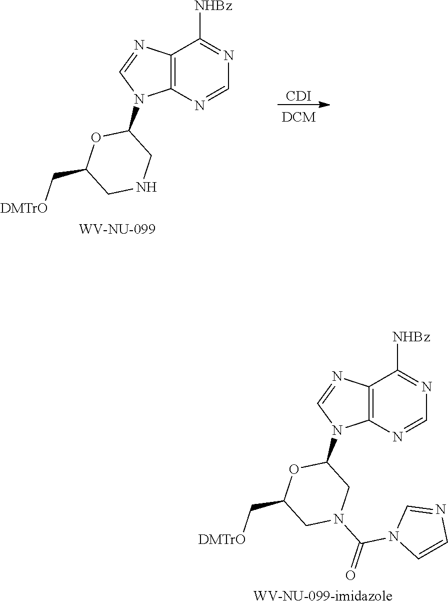

To a solution ofWV-NU-099 (6.5 g, 9.90 mmol) in DCM (200 mL) was added DIEA (1.92 g, 14.85 mmol), and CDI (2.41 g, 14.85 mmol). The mixture was stirred at 25° C. for 3 hr. LCMS showed WV-NU-099 was consumed completely and one major peak with desired mass was detected. The reaction mixture was quenched by H₂O (300 mL) and diluted with DCM (100 mL). This mixture was then extracted with DCM (100 mL * 2). The combined organic layers were washed with H₂O (200 mL), dried over Na₂SO₄, filtered and concentrated under reduced pressure to afford crude residue. The residue was purified by column chromatography (SiO₂, Petroleum ether/Ethyl acetate=2/1 to Ethyl acetate: ACN=5/1). WV-NU-099-imidazole (13.6 g, 88.02% yield, 96.192% purity) was obtained as a white solid. ¹FINMR (400 MHz, CHLOROFORM-d) δ=8.78 (s, 1H), 8.24 (s, 1H), 8.06-7.97 (m, 3H), 7.77-7.71 (m, 4H), 7.66-7.60 (m, 1H), 7.57-7.48 (m, 2H), 7.39 (d, J=7.3 Hz, 2H), 7.27 (s, 8H), 7.12 (s, 8H), 6.82 (dd, J=2.1, 8.9 Hz, 4H), 6.03 (dd, J=2.8, 10.3 Hz, 1H), 4.57 (br d, J=12.8 Hz, 1H), 4.21 (br d, J=13.5 Hz, 1H), 4.13-4.03 (m, 1H), 3.79 (d, J=1.0 Hz, 6H), 3.64 (dd, J=10.4, 13.1 Hz, 1H), 3.42 (dd, J=4.6, 10.1 Hz, 1H), 3.28 (dt, J=4.4, 10.1 Hz, 2H). LC-MS (M−H⁺): 749.3; LCMS purity 96.19%.

### Example 9. Synthesis of WV-NU-109A

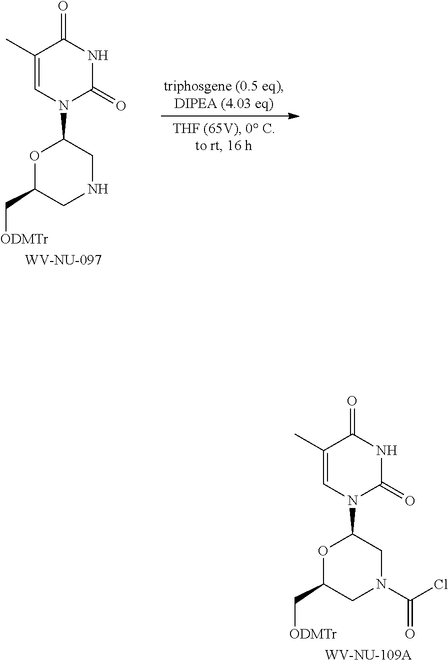

To a solution of triphosgene (1.64 g, 5.52 mmol) and DIEA (5.71 g, 44.15 mmol) in THF (300 mL) was added WV-NU-097 (6 g, 11.04 mmol) in THF (60 mL) at 0° C. under N₂. The mixture was stirred at 25° C. for 12 hr. TLC indicated WV-NU-097 was consumed completely and new spots formed. The reaction was quenched by sat. aq. NaHCO₃ (100 mL) and then extracted with EtOAc (50 mL * 2). The combined organic phase was washed with brine (100 mL), dried over anhydrous Na₂SO₄, filtered and concentrated in vacuo. Compound WV-NU-109A (4.7 g, 40.49% yield, 57.632% purity) was obtained as a yellow solid. LCMS: (M−H⁺)=604.2; Purity: 57.63%. TLC (Petroleum ether: Ethyl acetate=1: 1) R_{f}=0.30.

### Example 10. Synthesis of WV-DL-109C

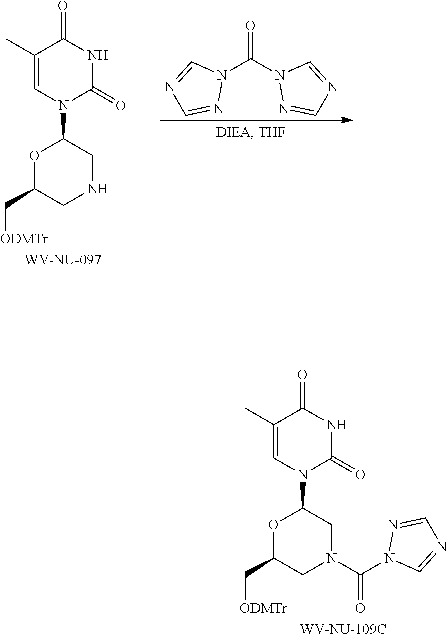

To a solution of compound WV-NU-097 (5 g, 9.20 mmol) in DCM (50 mL) was added bis(1,2,4-triazol-4-yl)methanone (2.26 g, 13.80 mmol) and DIEA (1.78 g, 13.80 mmol, 2.40 mL). The mixture was stirred at 25° C. for 1 hr. TLC indicated compound WV-NU-097 was consumed completely and new spots formed. The mixture was concentrated in vacuo. The residue was purified by column chromatography (SiO₂, Petroleum ether/Ethyl acetate=20/1 to 10/1, 5/1, 1/1, 1/2, 1/0). Compound WV-NU-109C (3.2 g, crude) was obtained as a white solid. ¹H NMR (400 MHz, CHLOROFORM-d) δ=9.31-9.11 (m, 1H), 8.90-8.84 (m, 1H), 8.23 (s, 4H), 8.05 (s, 1H), 7.44 (d, J=7.3 Hz, 2H), 7.35-7.28 (m, 7H), 7.26-7.21 (m, 1H), 6.88-6.79 (m, 4H), 5.87 (dd, J=2.8, 10.1 Hz, 1H), 4.11-4.07 (m, 1H), 3.82-3.78 (m, 6H), 3.38 (dd, J=4.6, 9.9 Hz, 1H), 3.30-3.19 (m, 1H), 3.15-3.06 (m, 2H), 1.97 (d, J=1.0 Hz, 3H). ¹³C NMR (101 MHz, CHLOROFORM-d) δ=163.72, 158.70, 152.43, 149.91, 148.58, 147.07, 144.42, 135.51, 135.46, 134.70, 130.04, 128.05, 127.95, 127.07, 113.26, 113.15, 111.72, 86.43, 79.02, 75.72, 63.52, 60.50, 55.28, 21.10, 14.22, 12.66. LCMS: M−H⁺=637.3; Purity: 98.73%. TLC (Petroleum ether: Ethyl acetate=1: 1) R_{f}=0.10.

### Example 11. Synthesis of Morpholine N-Carbamate (M-C) Dimers

Either the purified or crude carbamate (1.25 equiv), DBU (5 equiv.), and alcohol (1 equiv.) in anhydrous acetonitrile was stirred at 25° C. Stirring was continued for the reported time. The mixture was concentrated in vacuo and the residue was purified by silica gel column chromatography. Reaction conditions for representative morpholino N-carbamates are shown in Table 4 below. As demonstrated, various reagents may be useful. In some embodiments, reagents of entry 4 provides sufficient stability (e.g., for column purification), purity, and reactivity, and are utilized for oligonucleotide production.

TABLE 4
Certain results for certain preparations.

| Entry | R—H (connected through NH or OH if available) | 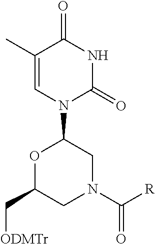 | Yield (%) If isolated. | Time (h) | 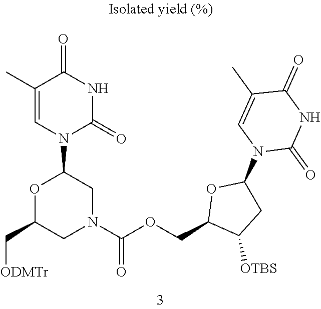 |
|---|---|---|---|---|---|
| 1 | 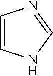 | 300 mg | 400 mg 68.2% yield | 5 h | LCMS_1B (reaction) 0.3 g 47.8 % yield 74.11% purity The residue was purified by RP-MPLC(MeCN/H₂O) |
| 2 | 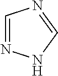 | 300 mg | 350 mg 59.8% yield | 5 h | LCMS_2B(reaction) 0.13 g 39.4% yield 96.5% purity The residue was purified by silica gel chromatography (Petroleum ether/Ethyl acetate = 10/1, 1/1) |
| 3 | 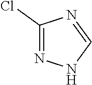 | 300 mg | 360 mg crude | 7 h | 190 mg 98.3% purity about 50.2% yield The residue was purified by RP-MPLC(MeCN/H₂O) |
| 4 | 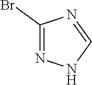 | 300 mg | 390 mg crude | 10-30 min | 160 mg 93% purity about 37.1% yield The residue was purified by RP-MPLC(MeCN/H₂O) |
| 5 | 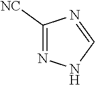 | 300 mg | 390 mg crude | 7 h | 80 mg 74% purity about 18% yield The mixture was purified by silica gel chromatography (Petroleum ether/Ethyl acetate = 10:1,1:1,0:1). |
| 6 | 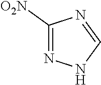 | 500 mg | 628 mg crude | 7 h | 0.38g 75% purity about 39% yield The residue was purified by column chromatography (SiO₂, Petroleum ether/Ethyl acetate = 3/1 to 0/1) |
| 7 | Cl | 500 mg | Used directly | 3 h | 280 mg 64% purity about 27% yield |
| | | | | | 0.38g 75% purity about39%yield |
| | | | | | The residue was purified by column chromatography |
| | | | | | (SiO₂, Petroleum ether/Ethyl acetate = 3/1 to 0/1) |
| 8 | HOSu | 500 mg | 0.62 g | 7 h | 220 mg 55% purity about 20%yield |
| | | | crude | | The mixture was purified by silica gel chromatography |
| | | | | | (Petroleum ether/Ethyl acetate = 10:1,1:1,0:1). |

¹H NMR (400 MHz, CHLOROFORM-d) δ = 9.78-9.43 (m, 1H), 7.52-7.17 (m, 13H), 6.80 (br d, J = 8.1 Hz, 5H), 5.96 (br s, 1H), 5.69 (br s, 1H), 4.52-3.85 (m, 8H), 3.81-3.68 (m, 7H), 3.26 (br d, J = 5.1 Hz, 1H), 3.19-3.05 (m, 1H), 2.79 (br s, 3H), 2.28 (br s, 2H), 1.89 (br d, J = 10.3 Hz, 7H), 0.85 (s, 9H), 0.04 (s, 6H). ¹³C NMR (101 MHz, CHLOROFORM-d) δ = 178.19, 110.18, 88.96, 80.77, 78.43, 76.60, 73.31, 65.55, 56.65, 53.83. LCMS: M − H⁺ = 924.4. Purity: 90.96%. TLC (Petroleum ether: Ethyl acetate = 0:1) R_{f} = 0.51.

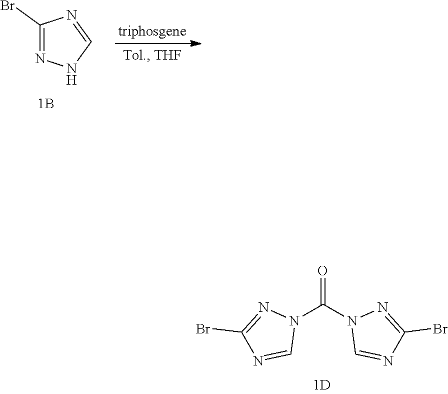 [SMILES: Brc1nc[nH]n1 ; O=C(n1cnc(Br)n1)n1cnc(Br)n1]

To a solution of bis(trichloromethyl) carbonate (9.99 g, 33.66 mmol, 0.083 eq.) in toluene (300 mL) was dropped compound 1B (60 g, 405.51 mmol, 1 eq.) dissolved in THF (300 mL) at 20° C. within 10 min and the mixture was stirred at 20° C. for 10 min. The cake was separated out from the solution. The mixture was filtered and the filtrated was concentrated to get the compound 1D (32 g, crude) as a white solid.

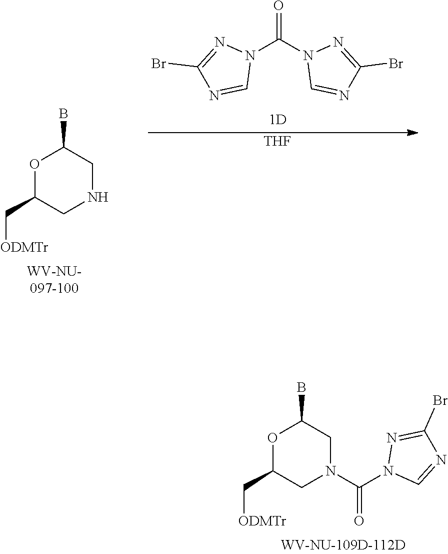

To a solution of Morpholine amine WV-NU-097-100 (63.22 mmol, 1 eq.) and DIEA (94.83 mmol, 16.52 mL, 1.5 eq.) in DCM (400 mL) was added compound 1D (94.83 mmol, 1.5 eq.) , the mixture was stirred at 20° C. for 12 hr. LCMS showed WV-NU-097-100 was consumed and the desired substance was found. The mixture was concentrated to get the crude product. The mixture was purified by silica gel chromatography (Petroleum ether/Ethyl acetate (5%TEA) to get WV-NU-109D-112D as a white solid.

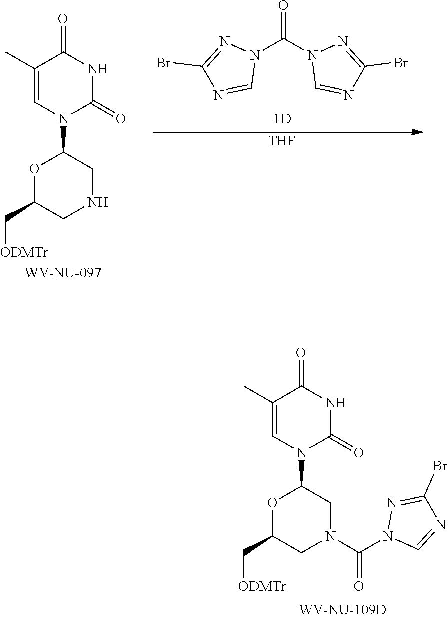

WV-NU-109D (19 g, 23.92 mmol, 43.84% yield, 90.35% purity) was obtained as a white solid. ¹HNMR (400 MHz, CHLOROFORM-d) δ=8.74 (s, 1H), 7.44 (br d, J=7.5 Hz, 2H), 7.36-7.27 (m, 7H), 7.27 (s, 2H), 6.85 (d, J=8.6 Hz, 5H), 5.84 (dd, J=2.6, 10.2 Hz, 1H), 4.10-4.04 (m, 1H), 3.80 (s, 6H), 3.39 (br dd, J=4.4, 9.8 Hz, 1H), 3.23 (br s, 1H), 3.15-3.05 (m, 2H), 1.96 (s, 3H). LCMS : (MS−H+): 715.2, purity: 94.70%.

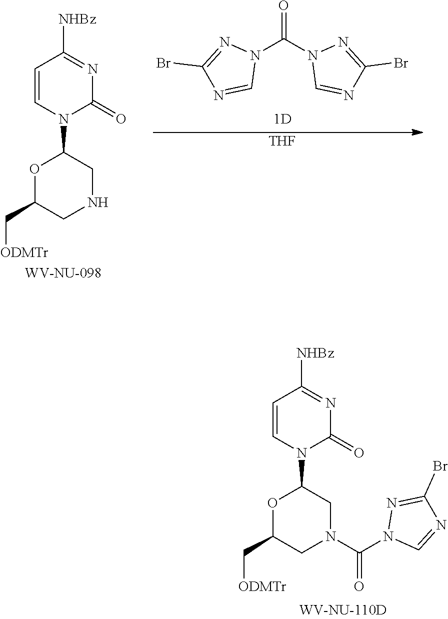

WV-NU-110D (15 g, 17.56 mmol, 27.78% yield, 94.437% purity) as a white solid. ¹HNMR (400 MHz, CHLOROFORM-d) δ=8.66 (br s, 1H), 7.95-7.77 (m, 3H), 7.61-7.33 (m, 5H), 7.29-7.12 (m, 8H), 6.77 (br d, J=7.9 Hz, 4H), 5.84 (br d, J=8.3 Hz, 1H), 4.81 (br s, 1H), 4.04 (br d, J=6.9 Hz, 2H), 3.72 (br s, 6H), 3.43-3.15 (m, 2H), 3.14-2.96 (m, 2H), 0.00-0.00 (m, 1H). LCMS (M−H+):806.2, LCMS purity: 94.437%. TLC (Petroleum ether : Ethyl acetate=0:1), Rf=0.56.

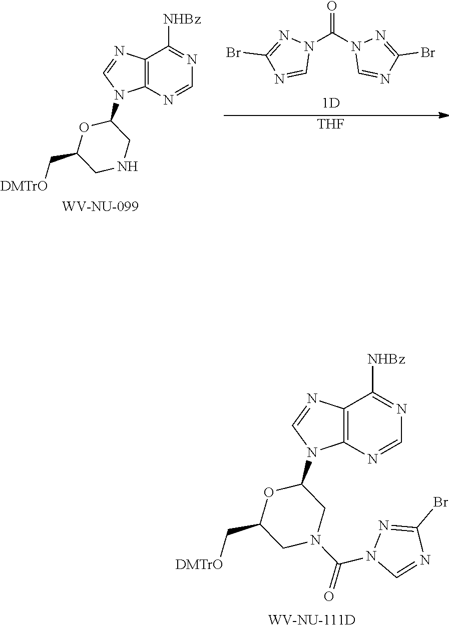

WV-NU-110D (16 g, 18.19 mmol, 36.91% yield, 94.464% purity) as a white solid. ¹HNMR (400 MHz, CHLOROFORM-d) δ=8.74 (s, 1H), 8.69 (s, 1H), 8.16 (d, J=3.9 Hz, 2H), 8.02-7.79 (m, 2H), 7.60-7.50 (m, 1H), 7.49-7.42 (m, 2H), 7.35 (d, J=7.4 Hz, 2H), 7.27-7.09 (m, 8H), 6.76 (dd, J=1.3, 8.8 Hz, 4H), 5.98 (br d, J=8.1 Hz, 1H), 4.13-4.07 (m, 1H), 3.71 (s, 6H), 3.42-3.29 (m, 1H), 3.19 (br dd, J=11.1, 13.6 Hz, 2H). LCMS purity: 96.538%, (M−H+):830.2.

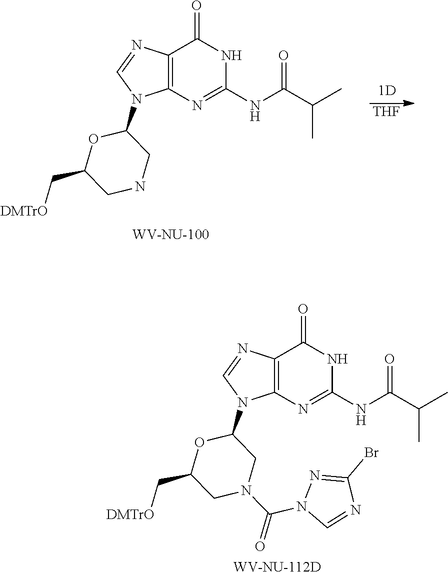

WV-NU-112D (17 g, 19.86 mmol, 34.75% yield, 94.929% purity) was obtained as a yellow solid. ¹HNMR (400 MHz, CHLOROFORM-d) δ ppm 1.27 (s, 5H) 2.63 (dt, J=13.57, 6.85 Hz, 1H) 2.88 (q, J=7.38 Hz, 1H) 3.23 (br dd, J=13.51, 11.13 Hz, 2H) 3.34-3.49 (m, 3H) 3.79 (s, 6H) 4.10 (s, 1H) 5.69 (br d, J=9.38 Hz, 1H) 6.83 (dd, J=8.82, 1.81 Hz, 4H) 7.19-7.35 (m, 8H) 7.43 (d, J=7.38 Hz, 2 H) 7.83 (s, 1H) 8.21 (s, 1H) 8.80 (s, 1H). LCMS: (M−H⁺): 812.2, purity: 94.929%. TLC (Petroleum ether : Ethyl acetate=0:1, R_{f}=0.35).

### Example 12. Synthesis of WV—SM-047a

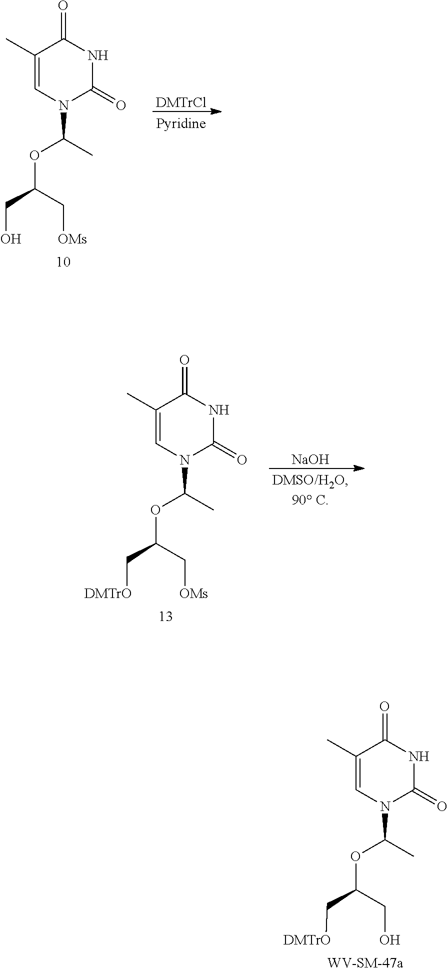

To a solution of compound 10 (10 g, 31.02 mmol) in pyridine (30 mL) was added DMTC1 (12.61 g, 37.23 mmol). The mixture was stirred at 15° C. for 4 hr. TLC indicated compound 10 was consumed and one new spot formed. The reaction mixture was diluted with sat. NaHCO3 (aq., 100 mL) and extracted with EtOAc (200 mL * 5). The combined organic layers were dried over Na₂SO₄, filtered and concentrated under reduced pressure to give a residue. The residue was purified by column chromatography (SiO₂, Petroleum ether/Ethyl acetate=20/1 to 1: 5, 5% TEA). Compound 13 (19 g, 98.04% yield) was obtained as a yellow solid. ¹H NMR (400 MHz, DMSO-d6) δ=11.35 (s, 1H), 7.50 (d, J=0.9 Hz, 1H), 7.32-7.22 (m, 4H), 7.21-7.08 (m, 5H), 6.83 (dd, J=1.3, 8.8 Hz, 4H), 5.94 (q, J=6.0 Hz, 1H), 4.37-4.28 (m, 1H), 4.20 (dd, J=5.3, 11.0 Hz, 1H), 3.78-3.68 (m, 7H), 3.13 (s, 3H), 3.04-2.85 (m, 2H), 1.59 (s, 3H), 1.42 (d, J=6.1 Hz, 3H). LCMS: (M+Na⁺): 647.3, LCMS purity: 97.22%. TLC (Petroleum ether: Ethyl acetate=0: 1), Rf=0.65.

A mixture of compound 13 (10 g, 16.01 mmol), NaOH (7.68 g, 192.09 mmol) in DMSO (60 mL) and H₂O (60 mL) was degassed and purged with N2 for 3 times, and then the mixture was stirred at 90° C. for 16 hr under N2 atmosphere. LCMS and TLC showed the reaction was completed, and one main peak with desired MS 545 (NEG, M−H⁺) was found. The reaction mixture was quenched by addition EtOAc (200 mL), and then diluted with H₂O (200 mL) and extracted with EtOAc (200 mL * 4). The combined organic layers were washed with brine (200 mL), dried over Na₂SO₄, filtered and concentrated under reduced pressure to give a residue. The residue was purified by column chromatography (SiO₂, Petroleum ether/Ethyl acetate=20/1 to 1: 3, 5% TEA). Compound WV—SM-047a (5.30 g, 57.88% yield, 95.564% purity) was obtained as a white solid. ¹H NMR (400 MHz, DMSO-d6) δ=11.31 (s, 1H), 7.50 (s, 1H), 7.31-7.21 (m, 4H), 7.21-7.08 (m, 5H), 6.83 (dd, J=2.2, 8.8 Hz, 4H), 5.96 (q, J=5.9 Hz, 1H), 4.73 (t, J=5.4 Hz, 1H), 3.71 (s, 6H), 3.54-3.45 (m, 1H), 3.37 (br d, J=2.9 Hz, 1H), 2.99-2.84 (m, 2H), 2.52 (s, 1H), 1.58 (s, 3H), 1.41 (d, J=6.0 Hz, 3H). ¹³C NMR (101 MHz, DMSO-d6) δ=163.86, 157.96, 150.92, 144.94, 135.82, 135.66, 135.54, 129.54, 129.48, 127.74, 127.51, 126.52, 113.11, 109.76, 85.29, 79.92, 78.34, 63.72, 60.59, 54.98, 20.76, 12.10. LCMS: (M−H⁺): 545.0, LCMS purity: 97.39%. HPLC: HPLC purity: 95.56%. Chiral SFC: 100% purity. TLC (Petroleum ether: Ethyl acetate=0: 1, 5% TEA), Rf=0.29.

### Example 13. Synthesis of WV—SM-10

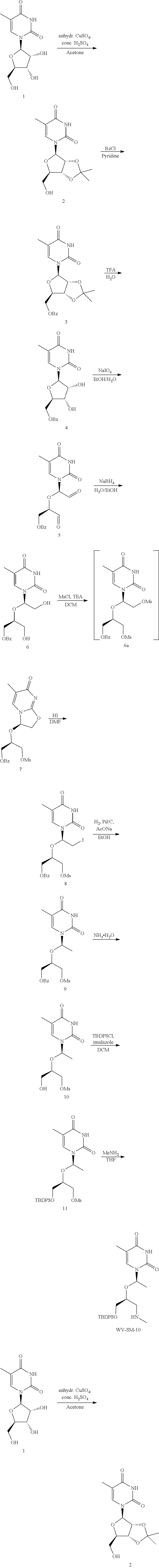

Four batches: To a solution of compound 1 (350 g, 1.36 mol) in acetone (2500 mL) was added CuSO₄ (700.00 g, 4.39 mol) and H2SO₄ (16.10 g, 164.15 mmol, 8.75 mL). The mixture was stirred at 15° C. for 24 hr. TLC indicated compound 1 was consumed and one new spot formed. Four batches: The reaction mixture was filtered, and the filtrate was then neutralized with NaHCO3 (powder) to pH=8, and then filtered, and concentrated under reduced pressure to give a crude product. Compound 2 (1.76 kg, crude) was obtained as a yellow oil. TLC (Dichloromethane: Methanol=9: 1), Rf=0.85.

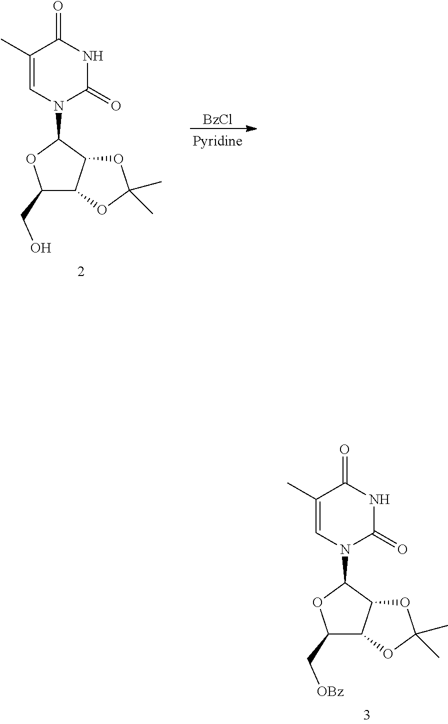

Four batches: To a solution of compound 2 (400 g, 1.34 mol) in pyridine (1700 mL) was added BzCl (282.74 g, 2.01 mol) in pyridine (800 mL). The mixture was stirred at 15° C. for 5 hr. TLC indicated compound 2 was consumed and two new spots formed. Four batches: The reaction mixture was concentrated under reduced pressure to remove pyridine. The residual solid was added EtOAc (1000 mL), and washed with sat. NaHCO3 (aq., 500 mL). The mixture was filtered, and the solid phase was desired product. Compound 3 (2.8 kg, crude) was obtained as a white solid. ¹H NMR (400 MHz, DMSO-d6) δ=7.95 (br d, J=7.5 Hz, 2H), 7.71-7.58 (m, 1H), 7.56-7.47 (m, 2H), 7.43 (s, 1H), 5.80 (s, 1H), 5.08-4.87 (m, 2H), 4.60-4.49 (m, 1H), 4.46-4.37 (m, 1H), 4.33 (br s, 1H), 1.59 (s, 3H), 1.51-1.46 (m, 1H), 1.48 (s, 2H), 1.29 (s, 3H). TLC (Ethyl acetate: Petroleum ether=3:1), R_{f}=0.75.

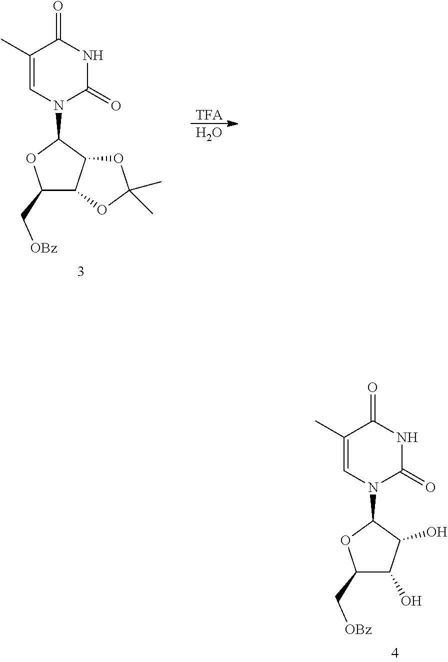

Four batches: compound 3 (540 g, 1.34 mol) was dissolved in TFA (2.31 kg, 20.26 mol) and H₂O (300 mL). And the solution was stirred for 10 hr at 15° C. TLC indicated compound 3 was consumed and one new spot formed. The reaction mixture was concentrated under reduced pressure to remove solvent. The residue was further recrystallized in EtOAc (500 mL) and filtered. Compound 4 (1. 6 kg, 51.42% yield) was obtained as a white solid. ¹H NMR (400 MHz, DMSO-d6) δ=11.33 (s, 1H), 8.03-7.93 (m, 2H), 7.71-7.63 (m, 1H), 7.57-7.48 (m, 2H), 7.35 (d, J=0.9 Hz, 1H), 5.79 (d, J=4.2 Hz, 1H), 4.56 (dd, J=3.1, 12.1 Hz, 1H), 4.46-4.36 (m, 1H), 4.18-4.06 (m, 3H), 1.57 (s, 3H). LCMS: (M+Na⁺): 384.9. TLC (Ethyl acetate: Petroleum ether=3:1), R_{f}=0.13.

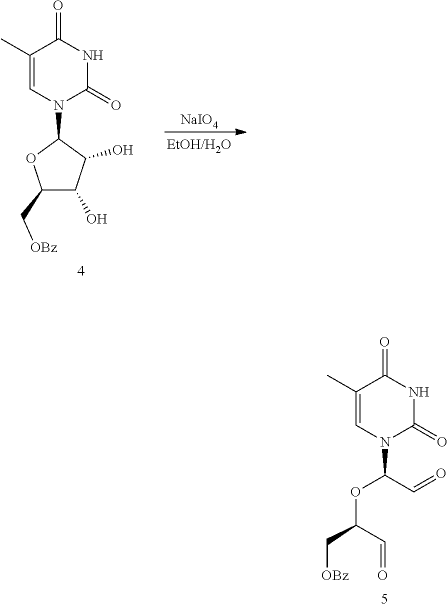

To a solution of compound 4 (50 g, 137.99 mmol) in EtOH (1000 mL) was added NaT0₄ (30.00 g, 140.26 mmol) in H₂O (500 mL). The mixture was stirred in dark at 15° C. for 2 hr. TLC indicated compound 4 was consumed and one new spot formed. Compound 5 (49.72 g, crude) was obtained as a white suspension liquid, which was used next step. TLC (Ethyl acetate: Methanol=9:1), Rf=0.49.

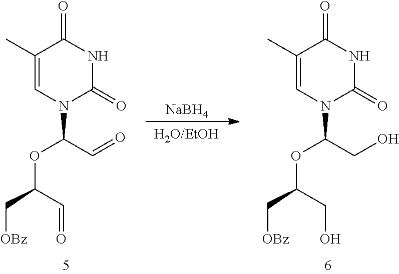

To a stirred solution of compound 5 (49.72 g, 137.99 mmol) in EtOH (1000 mL) and H₂O (500 mL) from the last step was added NaBH₄ (10.44 g, 275.98 mmol) in small portions at 0° C. The mixture was stirred at 15° C. for 1 hr. TLC indicated compound 5 was consumed and one new spot formed. The solvent was removed to yield a brown solid. The solid was added sat. Na₂SO₃ (aq., 500 mL), and then extracted with EtOAc (500 mL*5). The combined organic phase was dried by Na₂SO₄. Removal of the solvent under reduced pressure gave the product. Compound 6 (37.2 g, 73.99% yield, - purity) was obtained as a white solid. LCMS: (M+Na⁺): 386.9; TLC (Ethyl acetate: Methanol=9: 1), Rf=0.38.

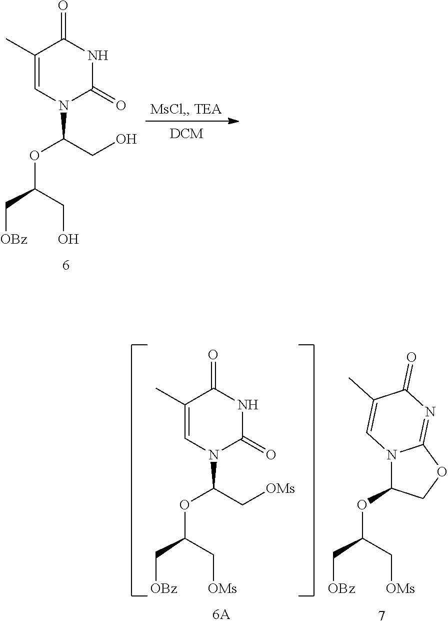

To a solution of compound 6 (33.7 g, 92.49 mmol) and TEA (46.80 g, 462.47 mmol) in DCM (300 mL) was added MsCl (23.31 g, 203.49 mmol) in DCM (150 mL). The mixture was stirred at 0° C. for 4 hr. TLC indicated compound 6 was consumed, and two new spots formed. The reaction mixture was quenched by addition water (100 mL), and stayed for 36 hr. TLC indicated compound 6A was consumed, and one spot (compound 7) left. The water layer was extracted with DCM (500 mL * 3). The combined organic layers were dried over Na₂SO₄, filtered and concentrated under reduced pressure to give a residue. The residue was purified by column chromatography (SiO₂, Petroleum ether/Ethyl acetate=20/1 to 0: 1). Compound 7 (35 g, 89.16% yield) was obtained as a white solid. ¹FINMR (400 MHz, DMSO-d6) 6=7.96-7.86 (m, 2H), 7.77 (d, J=1.3 Hz, 1H), 7.70-7.64 (m, 1H), 7.55-7.43 (m, 2H), 6.09 (dd, J=1.3, 5.7 Hz, 1H), 4.77 (dd, J=5 .7 , 10.5 Hz, 1H), 4.66-4.59 (m, 1H), 4.56-4.44 (m, 3H), 4.41-4.29 (m, 2H), 3.27 (s, 3H), 1.59 (d, J=1.3 Hz, 3H). LCMS: (M+H⁺): 425.2. TLC Petroleum ether: Ethyl acetate=0:1, R_{f}=0.38; Ethyl acetate: Methanol=9: 1, R_{f}=0.13.

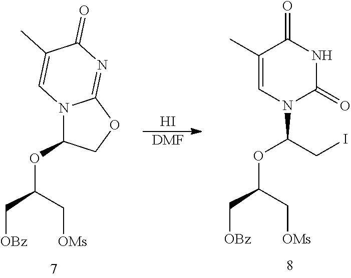

To a solution of compound 7 (36 g, 84.82 mmol) in DMF (300 mL) was added HI (48.22 g, 169.64 mmol, 28.36 mL, 45% purity). The mixture was stirred at 15° C. for 0.5 hr. TLC showed compound 7 was consumed and one main spot was detected. The reaction mixture was quenched by sat. NaHCO3 (aq.) to pH=7. The residue was extracted with EtOAc (500 mL * 3). The combined organic layers were washed with brine (500 mL), dried over Na₂SO₄, filtered and concentrated under reduced pressure to give a residue. Compound 8 (49.8 g, crude) was obtained as a brown oil. TLC (Ethyl acetate: Methanol=9:1), Rf=0.80.

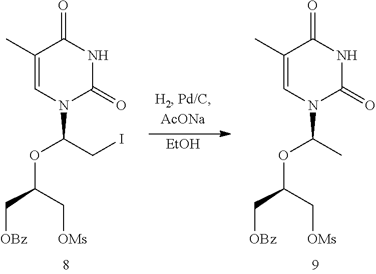

A mixture of compound 8 (46 g, 83.28 mmol), Pd/C (14 g, 10% purity) and NaOAc (62.10 g, 757.00 mmol) in EtOH (1000 mL) was degassed and purged with H₂ for 3 times, and then the mixture was stirred at 15° C. for 10 hr under H₂ atmosphere (15 psi). TLC and LC-MS showed compound 8 was consumed and one main spot was found. Pd/C was filtered off and the filtrate was evaporated. The residue was added with water (200 mL) and the water phase was extracted with EtOAc (300 mL*6). And then the organic layer was washed with brine (200 mL) and dried over Na₂SO₄, filtered and concentrated under reduced pressure to give a residue. The residue was purified by column chromatography (SiO₂, Petroleum ether/Ethyl acetate=20/1 to 1: 1). Compound 9 (30 g, 84.47% yield) was obtained as a colorless oil. ⁱH NMR (400 MHz, DMSO-d6) δ=11.30 (s, 1H), 7.91-7.81 (m, 2H), 7.71-7.62 (m, 1H), 7.54-7.44 (m, 3H), 6.04 (q, J=6.0 Hz, 1H), 4.55 (dd, J=3.7, 11.2 Hz, 1H), 4.40 (dd, J=4.8, 11.2 Hz, 1H), 4.34-4.21 (m, 2H), 4.18-4.10 (m, 1H), 3.28 (s, 3H), 1.49-1.42 (m, 6H). LCMS: (M+H⁺): 427.2. Chiral SFC: 100% purity. TLC (Petroleum ether: Ethyl acetate=1:3), Rf=0.12.

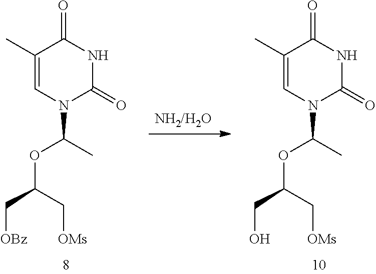

To a solution of compound 9 (30 g, 70.35 mmol) in MeOH (1000 mL) was added NH₃.H₂O (493.09 g, 3.52 mol, 541.86 mL, 25% purity). The mixture was stirred at 15° C. for 16 hr. TLC indicated compound 9 was consumed and one new spot formed. The reaction mixture was concentrated under reduced pressure to remove MeOH, and the water phase was extracted with EtOAc (300 mL * 8). The organic phase was dried with Na₂SO₄, filtered and concentrated under reduced pressure to give a residue. The residue was purified by column chromatography (SiO₂, Petroleum ether/Ethyl acetate=20/1 to 0: 1). Compound 10 (19 g, 83.79% yield) was obtained as a white solid. ¹H NMR (400 MHz, DMSO-d6) δ=11.27 (s, 1H), 7.55 (d, J=1.3 Hz, 1H), 5.92 (q, J=6.0 Hz, 1H), 4.83 (t, J=5.7 Hz, 1H), 4.37 (dd, J=3.3, 11.2 Hz, 1H), 4.20 (dd, J=5.3, 11.0 Hz, 1H), 3.64-3.54 (m, 1H), 3.33-3.29 (m, 2H), 3.20 (s, 3H), 1.78 (s, 3H), 1.39 (d, J=6.1 Hz, 3H). LCMS: (M+H⁺): 323.2, (M+Na⁺): 345.2. TLC (Ethyl acetate: Methanol=9:1), Rf=0.39.

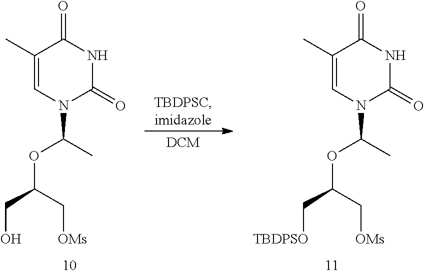

To a solution of compound 10 (9 g, 27.92 mmol) in DCM (150 mL) was added imidazole (4.56 g, 67.01 mmol) and TBDPSC1 (9.21 g, 33.51 mmol). The mixture was stirred at 15° C. for 4 hr. TLC indicated compound 10 was consumed and one new spot formed. The reaction mixture was quenched by addition water (100 mL), and the water phase was extracted with DCM (100 mL * 5). The combined organic layers were dried over Na₂SO₄, filtered and concentrated under reduced pressure to give a residue.

The residue was purified by MPLC (SiO₂, Petroleum ether/Ethyl acetate=19/1 to 1: 1). Compound 11 (15.2 g, 97.08% yield) was obtained as a white solid. ¹FINMR (400 MHz, DMSO-d6) δ=11.34 (s, 1H), 7.57 (br t, J=6.1 Hz, 4H), 7.51-7.34 (m, 7H), 5.99 (q, J=6.0 Hz, 1H), 4.56-4.29 (m, 2H), 3.84-3.73 (m, 1H), 3.65-3.50 (m, 2H), 3.22 (s, 3H), 1.61 (s, 3H), 1.43 (d, J=6.0 Hz, 3H), 0.95 (s, 9H). LCMS: (M+Na⁺): 583.2, LCMS purity: 95.16%. TLC (Ethyl acetate: Methanol=9: 1), Rf=0.72.

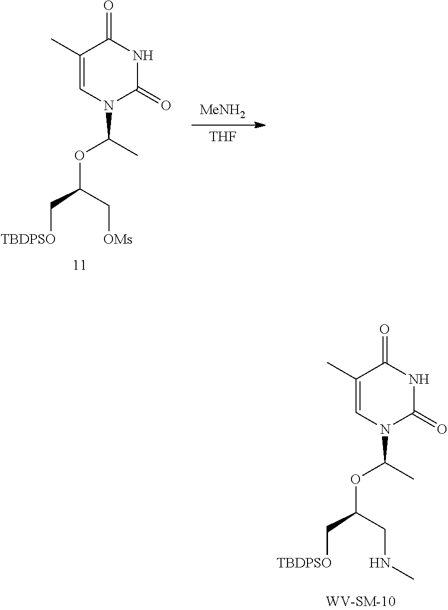

5 batches: To a solution of compound 11 (2.5 g, 4.46 mmol) was added MeNH₂ (2 M in THF, 85.00 mL). The mixture was stirred at 80° C. for 48 hr. TLC indicated compound 11 was remained a little and one new spot formed. The reaction mixture was concentrated under reduced pressure to remove solvent. The residue was purified by column chromatography (SiO₂, Petroleum ether/Ethyl acetate=20/1 to 1: 20). Compound WV—SM-10 (5.9 g, 51.93% yield, 97.262% purity) was obtained as a brown oil.

1H NMR (400 MHz, DMSO-d6) δ=7.61-7.52 (m, 4H), 7.49-7.36 (m, 7H), 5.98 (q, J=5.9 Hz, 1H), 3.65-3.48 (m, 3H), 2.73-2.53 (m, 3H), 2.32-2.23 (m, 3H), 1.65-1.58 (m, 3H), 1.46-1.36 (m, 3H), 0.99-0.91 (m, 9H). ¹³C NMR (101 MHz, DMSO-d6) δ=163.73, 150.85, 135.48, 134.96, 134.89, 132.88, 132.80, 129.83, 127.83, 109.82, 79.30, 77.32, 64.59, 51.62, 36.46, 26.48, 18.70, 12.08. LCMS: (M+H⁺): 496.3, LCMS purity: 97.26%. Chiral SFC: dr=97.05: 2.95. TLC (Ethyl acetate: Methanol=9:1), Rf=0.12.

### Example 14. Constructions of Certain Linkages

Experimental procedure (A) for stereopure PN-dimer 2001, 2002 and stereorandom dimer 2001/2002: To a stirred solution of amidite/thioite (0.29 mmol, 1.6 equiv., pre-dried by co-evaporation with dry acetonitrile followed by under vacuum for minimum 12 h) in dry acetonitrile (1.5 mL) was added a solution of 2-azido-1,3-dimethylimidazolinium hexafluorophosphate ((ADIH) 98 mg, 0.34 mmol, 1.9 equiv.) in acetonitrile (0.4 mL) under argon atmosphere at room temperature. Resulting reaction mixture was stirred for 10 min. then DMTr protected alcohol (0.18 mmol, pre-dried by co-evaporation with dry acetonitrile and dried under vacuum for minimum 12 h) in dry acetonitrile (1 mL) and 1,8-Diazabicyclo [5.4.0] undec-7-ene (0.55 mmol, 3 equiv., 0.55 ml of 1 M solution in dry acetonitrile) were added. After the reaction was completed (monitored by LCMS), the reaction mixture was concentrated under reduced pressure then re-dissolved in DCM, washed with aq. NaHCO3 followed by concentration of the organic layer gave the crude product (yield various from 55% to 90%) and all the products were analyzed by ³¹P NMR and LCMS.

Experimental procedure (B) for chloro reagent (2003, 2005 and 2007): Thio alcohol (82.12 mmol) was dissolved in toluene (100 mL, 250 mL single neck flask, water bath temperature=35 ° C.), then dried by co-evaporating with toluene followed by under high vacuum for 2-3 h. Then it was removed from vacuum and re-dissolved in dry toluene (100 ml). To the resulting solution 4-methylmorpholine (18.0 mL, 164.24 mmol) was added. This mixture was added dropwise via cannula over 30 min to an ice-cold solution of phosphorus trichloride (7.2 mL, 82.12 mmol) in toluene (100 mL). After warming to room temperature for 1 h, the mixture was filtered carefully under vacuum/argon. The resulting filtrate was concentrated by rotary evaporation (flushing with Ar) then dried under high vacuum overnight. The resulting crude compound was isolated as thick oil, which was dissolved in THF to obtain a 1 M stock solution and this solution was used in the next step without further purification.

Experimental procedure (C) for phosphorothoite (2004, 2006 and 2008): The 5′-ODMTr protected nucleoside (3 g, 5.50 mmol) was dried in a three neck 100 mL round bottom flask by co-evaporating with anhydrous toluene (50 mL) followed by under high vacuum for 18 h. The dried nucleoside was dissolved in dry THF (30 mL). Then, triethylamine (2.3 mL, 16.5 mmol, 3 equiv.), dried over CaH2, was added into the reaction mixture, then cooled to —-10° C. A THF solution of the crude chloro reagent (1 M solution, 16.5 mL, 3 equiv., 16.5 mmol) was added to the above mixture through cannula over —15 min, then, gradually warmed to room temperature over about 1 h. LCMS showed that the starting material was consumed. The reaction mixture was filtered carefully under vacuum/argon and the resulting filtrate was concentrated under reduced pressure to give a yellow foam which was further dried under high vacuum overnight. Crude mixture was purified by silica gel column [Column was pre-deactivated using acetonitrile then ethyl acetate (5% TEA) and then equilibrated using ethyl acetate-hexanes] chromatography using ethyl acetate and hexane as eluents. Yield ranges between 65% and 90%.

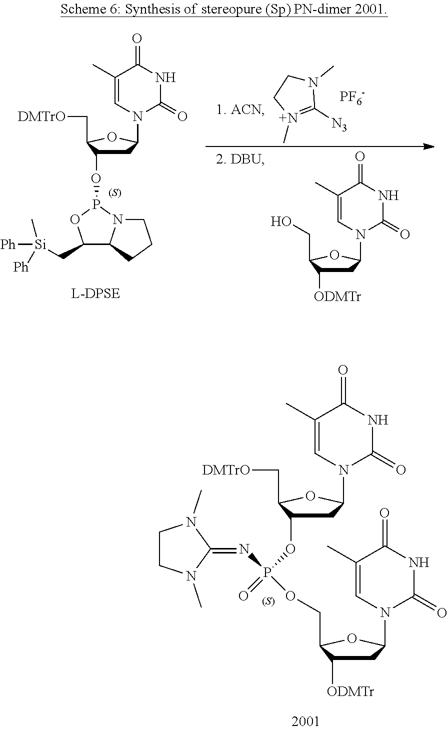

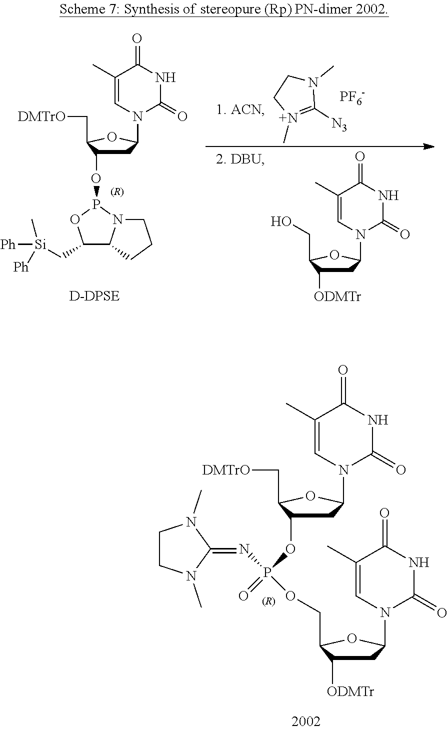

Compound 2001 (stereopure (Sp)): Procedure A was followed. L-DPSE chiral amidite was used. ³¹P NMR (162 MHz, CDCl₃) 6-1.82. MS (ES) m/z calculated for C67H72N7015P [M+Na]⁺1268.47, Observed: 1268.38 [M+Na]⁺.

Compound 2002 (stereopure (Rp)): Procedure A was followed. D-DPSE chiral amidite was used. ³¹P NMR (162 MHz, CDCl₃) 6-1.20. MS (ES) m/z calculated for C67H72N7015P [M+Na]⁺1268.47, Observed: 1268.48 [M+Na]⁺.

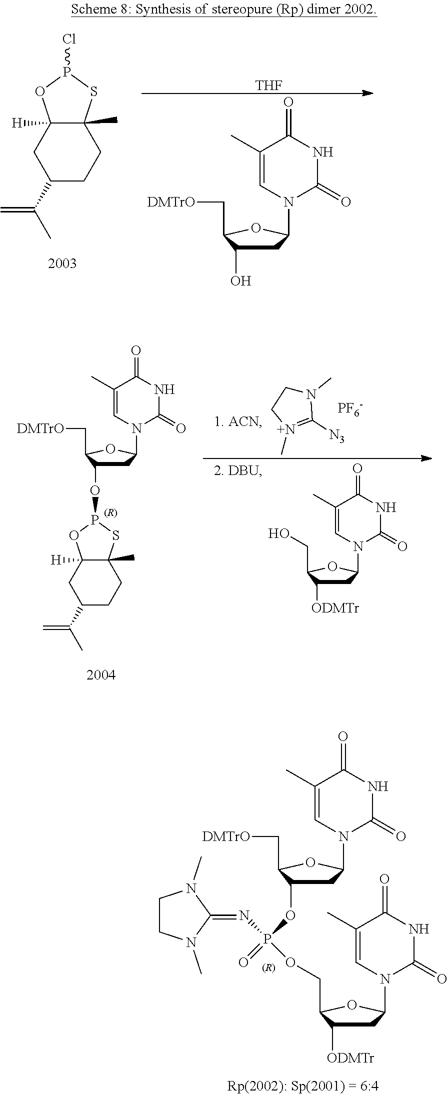

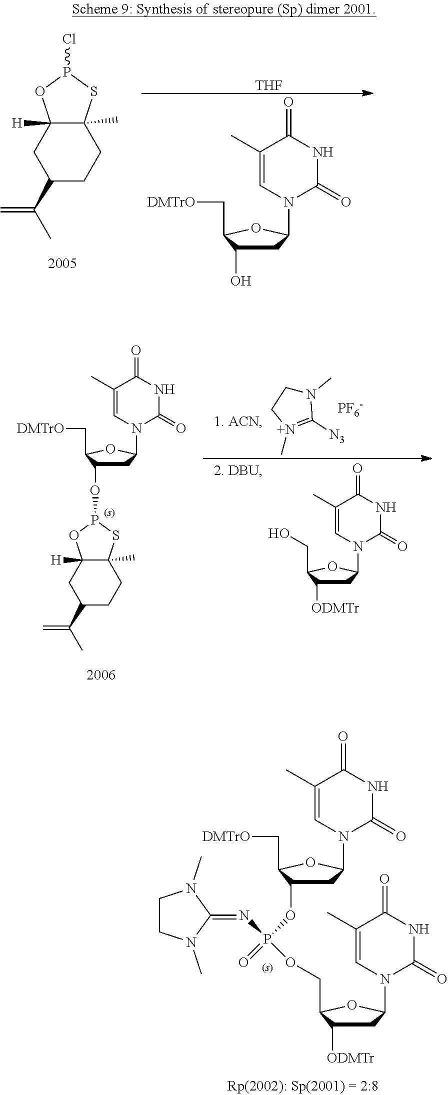

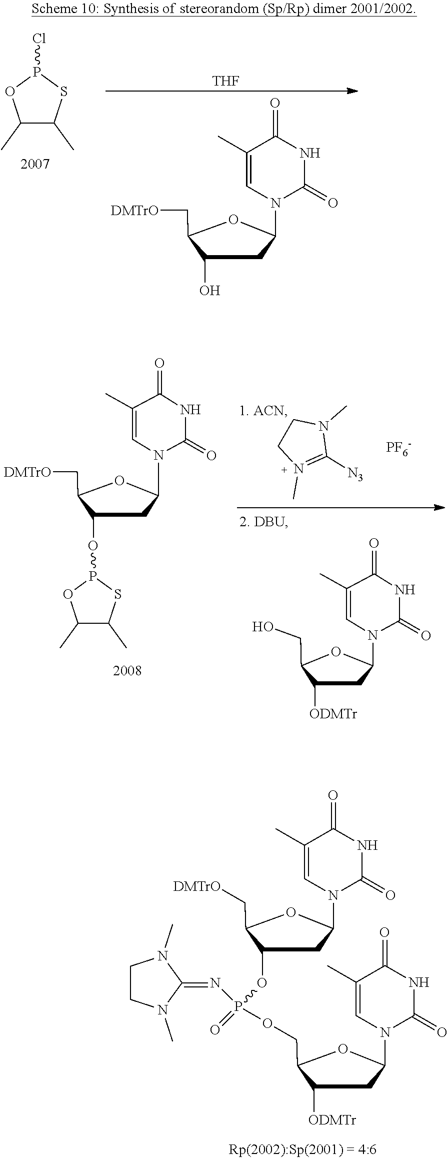

Compound 2003: Procedure B was followed. ³¹P NMR (162 MHz, THF-CDCl₃, 1:2) 6 207.89

Compound 2005: Procedure B was followed. ³¹P NMR (162 MHz, THF-CDCl₃, 1:2) 6 207.89

Compound 2007: Procedure B was followed. ³¹P NMR (162 MHz, THF-CDCl₃, 1:2) 6 205.92, 205.67, 205.53

Compound 2004 (stereopure (Sp): Procedure C was followed. ³¹P NMR (162 MHz, CDCl₃) 6 189.86 MS (ES) m/z calculated for C41t147N208PS [M+K]⁺797.24, Observed: 797.20 [M+K]⁺.

Compound 2006 (stereopure (Rp)): Procedure C was followed. ³¹P NMR (162 MHz, CDCl₃) δ 189.51 MS (ES) m/z calculated for C4II⁻147N208PS [M+K]⁺797.24, Observed: 797.20 [M+K]⁺.

Compound 2008 (stereorandom): Procedure C was followed. ³¹P NMR (162 MHz, CDCl₃) 6 175.56, 174.79, 174.44, 173.85, 173.38, 172.90 MS (ES) m/z calculated for C₃₅H₃₉N₂O₈PS [M+K]⁺717.18, Observed: 717.22 [M+K]⁺.

Synthesis of PS/PO/PN 20mer on solid phase.

Abbreviations used:

1× solution: 1M HF-TEA in H₂O-DMSO (1:5, v/v)

Ac: acetyl

Ac₂O: acetic anhydride

ADIH: 2-azido-1,3-dimethyl-4,5-dihydro-1H-imidazol-3-ium hexafluorophosphate (V)

Cap-A: 20vol % MeIm in MeCN

Cap-B: Ac₂O-2,6-lutidine-MeCN (2:3:5, v/v/v)

CMIMT: N-cyanomethylimidazolium triflate

CPG: controlled pore glass

DCA: dichloroacetic acid

DCM: dichloromethane

DEA: diethylamine

DMTr: 4,4′-dimethoxytrityl

DMSO: dimethylsulfoxide

HF: hydrogen fluoride

HFIP: 1,1, 1,3,3,3-hexafluoro-2-propanol

MeCN: acetonitrile

Melm: N-methylimidazole

Ph: phenyl

RP-UPLC: reversed-phase ultra performance liquid chromatography

TEA: triethylamine

XH: xanthane hydride

Procedure for the solid-phase synthesis of oligonucleotide compositions containing P(V) chemistry: In an example, automated solid-phase synthesis (24 umol scale) of WV-27145 for chiral PS and PO linkages was performed using TWIST™ 10 um/15 um column (GlenResearch, catalog #20-0040) filled with 325 mg of N-methylated aminopropyl CPG which derivatized 2′F-dU by succinyl linker at 3′-O-position, according to the cycles shown in Table 5. For the stereo-random PN linkage, synthesis was performed using P(V) chemistry described in Table 6.

After completion of the automated oligonucleotide synthesis, the CPG support was treated with 20% DEA in MeCN for 12 min, washed with dry MeCN and dried under argon and vacuum. The dry CPG support was transferred into a 15 mL plastic tube, treated with 1X solution (100 uL/umol) for 6 h at 28° C., then added conc. NH₃ (aqueous solution, 200 uL/umol) and cooked for 24 h at 37° C. The mixture was cooled to room temperature and the CPG was removed by membrane filtration, and analyzed by LTQ and RP-UPLC with a linear gradient of MeCN (1-15%/15 min) in (10 mM TEA, 100 mM HFIP in water) at 55° C. at a rate of 0.8 mL/min. The crude WV-27145 was purified by AEX-HPLC eluting with 20 mM NaOH to 2.5M NaCl, and desalt to obtain the product.

TABLE 5
Certain Conditions.

| | | | | waiting |
|---|---|---|---|---|
| step | operation | reagents and solvent | volume | time |
| 1 | detritylation | 3% DCA/DCM | 10 mL | 1 min |
| 2 | coupling | 0.2M monomer/MeCN | 0.5 mL | 8 min |
| | | 0.6M CMIMT/MeCN | 1 mL | |
| 3 | cap-1 | Cap-B | 2 mL | 2 min |
| 4 | oxidation or | 50 mM I₂/pyridine-H₂O or | 2 mL | 1 min |
| | sulfurization or | 0.2M XH/pyridine or | 2 mL | 6 min |
| 5 | cap-2 | Cap-A + Cap-B | 2 mL | 45 s |

TABLE 6
Certain Conditions.

| | | | | waiting |
|---|---|---|---|---|
| step | Operation | reagents and solvent | volume | time |
| 1 | detritylation | 3% DCA/DCM | 10 mL | 1 min |
| | coupling | 0.2M monomer/MeCN* | 0.5 mL | 10 min |
| 2 | | 1M DBU/MeCN | 1 mL | |
| 3 | cap-2 | Cap-A + Cap-B | 2 mL | 45 s |

*Compound 2008 (407 mg, 0.6 mmol) was dissolved in 3 mL of anhydrous MeCN, added MS3Å (410 mg). After 30 min, added ADIH (205 mg, 0.72 mmol, generated N₂ gas) and mixed well.

Summary of results in an example: Crude ODs: 1648 ODs Crude UPLC Purity: 46.26% Crude Mass Purity: 61.90% Final ODs: 510 ODs Final UPLC Purity: 85.48% Final Mass Purity: 87.97% Observed Mass: 6965.1

### Example 15. Synthesis of WV-CA-299

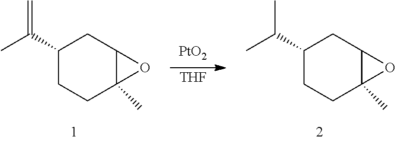

To a solution of compound 1 (15 g, 98.53 mmol, 1 eq.) in THF (300 mL) was added PtO2 (2.24 g, 9.85 mmol, 0.1 eq.). The mixture was stirred at 15° C. for 0.5 hr. HNMR showed compound 1 was consumed and the compound was desired. Filter out the PtO2, the residue was concentrated under reduced pressure to give a crude. The residue was purified by column chromatography (SiO₂, Petroleum ether/Ethyl acetate=1/0 to 0/1). Compound 2 (10 g, 64.83 mmol, 65.80% yield) was obtained as a yellow oil.

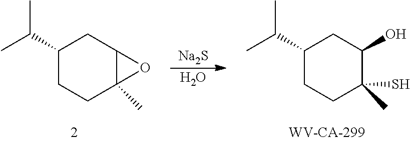

To a solution of compound 2 (9 g, 58.35 mmol, 1 eq.) in H₂O (180 mL) was added Na₂S (13.66 g, 175.04 mmol, 7.34 mL, 3 eq.). The mixture was stirred at 50-80° C. for 48 hr. TLC (Petroleum ether: Ethyl acetate=5:1, Rf=0.28) indicated compound 2 was consumed and two new spots formed. The reaction mixture was quenched by addition sat. NH₄Cl aq. until pH-8 at 0° C., extracted with EtOAc (100 mL×4), dried over Na₂SO₄, filtered and concentrated under reduced pressure to give a residue. The residue was purified by column chromatography (SiO₂, Petroleum ether/Ethyl acetate=1/0 to 0/1). Compound WV-CA-299 (4 g, 21.24 mmol, 36.40% yield) was obtained as a colorless oil. ¹H NMR (400 MHz, CHLOROFORM-d): δ=3.69 (br d, J=2.8 Hz, 1H), 1.86-1.74 (m, 2H), 1.71 (br d, J=4.1 Hz, 1H), 1.61-1.56 (m, 2H), 1.55 (br s, 2H), 1.46-1.38 (m, 1H), 1.36 (s, 3H), 1.32 (br d, J=10.0 Hz, 2H), 0.82 (dd, J=4.4, 6.6 Hz, 6H). ¹³C NMR (101 MHz, CHLOROFORM-d): δ=75.66, 60.38, 48.45, 36.89, 35.29, 32.48, 31.59, 25.31, 19.99, 19.81, 14.19. TLC: (Petroleum ether: Ethyl acetate=5:1) R_{f=}0.28.

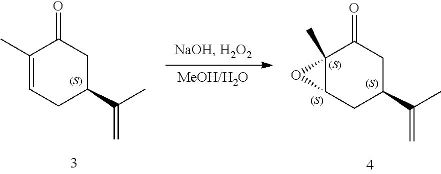

Compound 3 (30 g, 199.71 mmol, 31.09 mL, 1 eq.) was dissolved in MeOH (200 mL), H₂O₂ (65.67 g, 579.16 mmol, 55.65 mL, 30% purity, 2.9 eq.) was added. Sodium hydroxide (6 M, 66.57 mL, 2 eq.) was dropped under 0° C. and the mixture was stirred at 0° C. for 3 hr. TLC (Petroleum ether/Ethyl acetate=3:1, Rf=0.43) showed compound 3 was consumed. Water (300 mL) was added and extracted with MTBE (200 mL×2). The combined organic was washed with sat. aq. Na₂SO₃ (100 mL) and sat. aq. NaHCO3, (100 mL). The organic was dried over Na₂SO₄, filtered and concentrated to get the crude. The mixture was purified by silica gel chromatography (Petroleum ether/Ethyl acetate=10:1, 5:1) to get compound 4 (28 g, 168.46 mmol, 84.35% yield) as a colorless oil. ¹HNMR(400 MHz, CHLOROFORM-d): δ=4.84-4.67 (m, 2H), 3.44 (d, J=2.0 Hz, 1H), 2.76-2.65 (m, 1H), 2.57 (ddd, J=1.2, 4.5, 17.7 Hz, 1H), 2.40-2.32 (m, 1H), 2.08-1.96 (m, 1H), 1.89 (ddd, J=0.9, 11.2, 14.7 Hz, 1H), 1.70 (s, 3H), 1.45-1.36 (m, 3H). TLC: (Petroleum ether/Ethyl acetate=3:1), Rf=0.43.

### Example 16. Synthesis of WV-CA-299 and WV-CA-296A

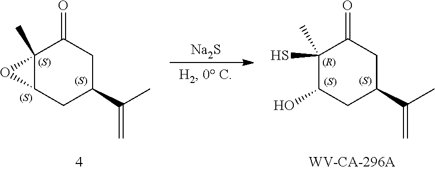

To a solution of compound 4 (28 g, 168.46 mmol, 1 eq.) in H₂O (600 mL) was added Na₂S (39.44 g, 505.37 mmol, 21.20 mL, 3 eq.). The mixture was stirred at 0° C. for 3 hr. TLC (Petroleum ether/Ethyl acetate=3:1) showed compound 4 was consumed and a new spot was found. The mixture was added NH₄Cl solid until pH about 7-8 and extracted with DCM (100 mL×3), dried overNa₂SO₄, filtered and concentrated to get the crude. The residue was purified by silica gel chromatography (Petroleum ether/Ethyl acetate=1/0 to 10/1) to get compound WV-CA-296A (27 g, 130.73 mmol, 77.60% yield, 96.98% purity) as a colorless oil.

¹HNMR (400 MHz, CHLOROFORM-d): δ=4.73 (br d, J=12.5 Hz, 2H), 4.15-4.07 (m, 1H), 3.09-2.94 (m, 1H), 2.86-2.72 (m, 1H), 2.36-2.26 (m, 2H), 2.19-2.09 (m, 1H), 2.02 (s, 1H), 1.94-1.85 (m, 1H), 1.73-1.66 (m, 3H), 1.53-1.45 (m, 3H). LCMS purity: 96.98%, [1VI +H] +201.0. TLC: (Petroleum ether/Ethyl acetate=3:1), Rf=0.32.

### Example 17. Synthesis of WV-CA-292

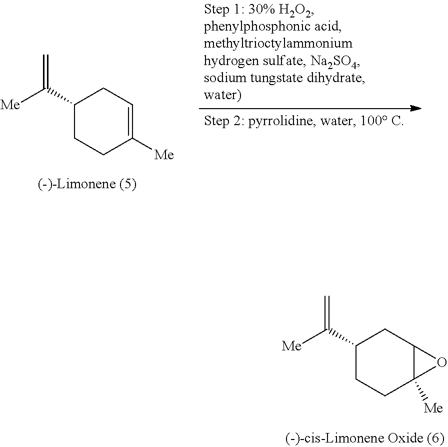

Two batches in parallel: To a 2 L three-neck flask was added H₂O₂ (486.81 g, 4.29 mol, 412.55 mL, 30% purity, 1.17 eq.), phenylphosphonic acid (5.80 g, 36.70 mmol, 0.01 eq.), hydrogen sulfate methyl(trioctyl)ammonium (34.19 g, 73.40 mmol, 0.02 eq.), Na₂SO₄ (156.39 g, 1.10 mol, 111.71 mL, 0.3 eq.), disodium dioxido(dioxo)-tungsten dihydrate (24.21 g, 73.40 mmol, 0.02 eq.) followed by H₂O (200 mL) at 20° C. To the stirred solution was slowly added (45)-4-isopropenyl-1-methyl-cyclohexene (5) (500 g, 3.67 mol, 588.24 mL, 1 eq.) dropwise keeping the temperature below 30° C., over 3 hours on an ice-water bath. Stirring at 30° C. for 18 hours. TLC (Petroleum ether) showed the reaction was completed.

The two batches reaction mixture was combined and diluted with hexane (1200 mL). The separated organic layer was washed with sodium bisulfite (400 mL, 10% aqueous), NaHCO₃ (400 mL, saturated aqueous), then brine (400 mL). The combined organic layers were dried over Na₂SO₄, filtered, then concentrated under reduced pressure. To the crude limonene oxide was added pyrrolidine (522 g, 612 mL, 1.0 eq.) then water (105.6 mL, 0.80 eq.). The reaction was stirred at 100° C. for 18 hours. The reaction was cooled to 25° C. and hexane (1000 mL) was added. The organics were washed with citric acid (20% aqueous, 1600 mL×4). The organics were washed with sat. NaHCO3 (300 mL) until pH >7, followed by a brine wash (300 mL). The compound was dried over Na₂SO₄, filtered, then concentration under reduced pressure to give a brown oil (300 g). The crude product was purified by column chromatography on silica gel (Ethyl acetate: Petroleum ether 0:1, 50:1, 20:1). Compound 6 (272 g, 1.79 mol, 24.34% yield) was obtained as a light-yellow oil. ¹H NMR (400 MHz, CHLOROFORM-d): δ=4.73 (d, J=1.4 Hz, 1H), 4.67 (s, 1H), 3.08-3.02 (m, 1H), 2.19-2.04 (m, 2H), 1.92-1.78 (m, 2H), 1.74-1.64 (m, 4H), 1.58-1.49 (m, 1H), 1.34-1.30 (m, 3H), 1.24-1.12 (m, 1H). TLC: (Petroleum ether) R_{f}=0.10.

Preparation of compound WV-CA-292 (Method I)

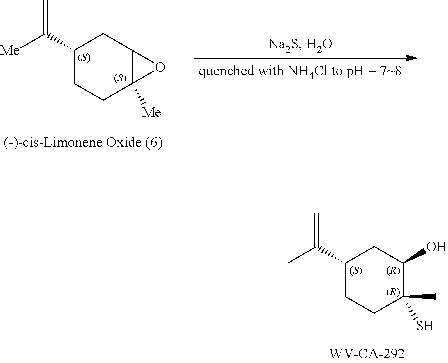

To a solution of compound (-)-cis-Limonene Oxide (6) (50 g, 328.44 mmol, 1 eq.) in H₂O (1000 mL) was added Na₂S (76.90 g, 985.33 mmol, 41.34 mL, 3 eq.). The mixture was stirred at 50—80 ° C. for 48 hr. TLC (Petroleum ether: Ethyl acetate=10:1, Rf=0.27) indicated compound (-)-cis-Limonene Oxide (6) was consumed and two new spots formed. The reaction mixture was added sat. aq. NH₄Cl until pH=7—8, the reaction mixture was added DCM (100 mL) and extracted with DCM (100 mL×3), dried over Na₂SO₄, filtered and concentrated under reduced pressure to give a residue. The residue was purified by column chromatography (SiO₂, Petroleum ether/Ethyl acetate=1/0 to 1/1). Compound WV-CA-292 (23.7 g, 127.20 mmol, 38.73% yield) was obtained as a colorless oil. ¹HNMR (400 MHz, CHLOROFORM-d): 6=4.88-4.66 (m, 2H), 3.80 (br d, J=1.9 Hz, 1H), 2.34-2.23 (m, 1H), 2.09 (ddd, J=2.6, 11.7, 14.0 Hz, 1H), 1.99-1.81 (m, 2H), 1.79-1.51 (m, 9H), 1.45 (s, 3H). ¹³CNMR (101 MHz, CHLOROFORM-d): δ =149.05, 109.24, 109.15, 75.41, 60.45, 48.13, 37.71, 35.22, 33.88, 29.59, 29.49,29.32, 27.22, 22.67, 21.05, 20.99, 20.95, 14.19. GCMS: MS=186. TLC: (Petroleum ether: Ethyl acetate=10:1), R_{f=}0.27.

### Example 18. Synthesis of WV-CA-292A

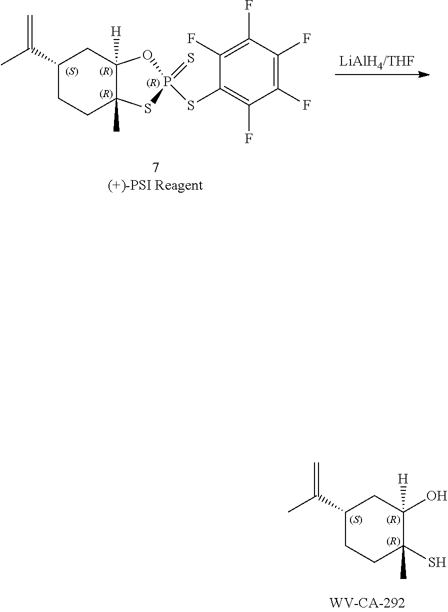

To a solution of LiA1H₄ (10 mL, 2M in THF) in THF (40 mL) cooled to -20° C. (dry ice-bath), a solution of (+)—PSI reagent (7), (9 g, 22.4 mool) in THF (60) was added drop-wise and ice-bath was removed and stirred at rt 1-1.5 h (Reaction mixture becomes slightly pinkish color). After completion of reaction (TLC monitoring) cooled to 0° C., then quenched with MeOH (2 eq. 2 mL) and solvents were evaporated to give residue, to this residue water was added and filtered with celite. The filtrate was extracted with EtOAc (250×2) and combined organic phase was dried over Na₂SO₄ and concentrated to give colorless oil, which was purified by Combiflash (80 g redsep high performance silica column) using EtOAc/Hexanes a solvent (compound eluted 30-40% of EtOAc in Hexanes). After evaporation of column fractions pooled together was dried (1 h) under vacuum to give WV-CA-292 as a colorless oil (isolated yield 85%). Analytical data was identical with Method I.

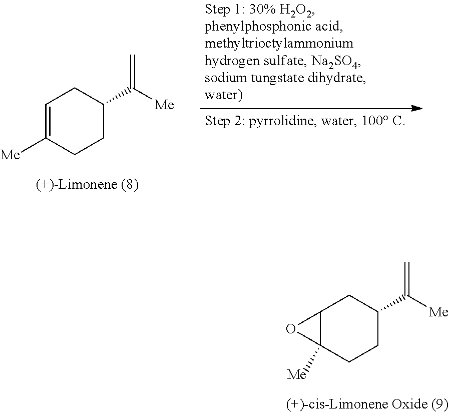

To a 2 L three-neck flask was added H₂O₂ (540.69 g, 4.77 mol, 458.21 mL, 30% purity, 1.15 eq.) , phenylphosphonic acid (6.56 g, 41.47 mmol, 0.01 eq.), hydrogen sulfate methyl(trioctyl)ammonium (38.63 g, 82.95 mmol, 0.02 eq.), Na₂SO₄ (176.72 g, 1.24 mol, 126.23 mL, 0.3 eq.), disodiumdioxido(dioxo)tungsten dihydrate (27.36 g, 82.95 mmol, 0.02 eq.) followed by H₂O (240 mL) at 20° C. To the stirred solution was slowly added (4R)-4-isopropenyl-1-methyl-cyclohexene (8) (565 g, 4.15 mol, 1 eq.) keeping the temperature below 30° C., over 2 hours. Stirring at 30° C. for 18 hours. TLC (Petroleum ether) showed the reaction was completed. The reaction was diluted with hexane (600 mL). The separated organic layer was washed with sodium bisulfite (250 mL, 10% aqueous), NaHCO₃ (250 mL, saturated aqueous), then brine (250 mL). The combined organic layers were concentrated under reduced pressure. To the crude limonene oxide was added pyrrolidine (346 mL, 1.0 eq.) then water (60 mL, 0.80 eq.). The reaction was stirred at 100° C. for 26 hours (took a sample and detected by HNMR, it showed the product clean). The reaction was cooled to 25° C. and hexane (500 mL) was added. The organics were washed with citric acid (20% aqueous, 800 mL×4). The organics were washed with sat. NaHCO3 (300 mL) until pH >7, followed by a brine wash (300 mL). The compound was dried over Na₂SO₄, filtered, then concentration under reduced pressure to give a light brown oil (150 g). The crude was purified by column chromatography on silica gel (Ethyl acetate: Petroleum ether=0: 1, 1: 20, 10:1). Compound 9 (124 g, 814.54 mmol, 19.64% yield) was obtained as a crude light-yellow oil. ⁱH NMR (400 MHz, CHLOROFORM-d): δ=4.75-4.70 (m, 1H), 4.67 (s, 1H), 3.07-3.02 (m, 1H), 2.18-2.06 (m, 2H), 1.92-1.78 (m, 2H), 1.73-1.66 (m, 4H), 1.58-1.49 (m, 1H), 1.30 (s, 3H), 1.24-1.14 (m, 1H). TLC: (Petroleum ether) R_{f}=0.10.

Preparation of compound WV-CA-292A (Method I)

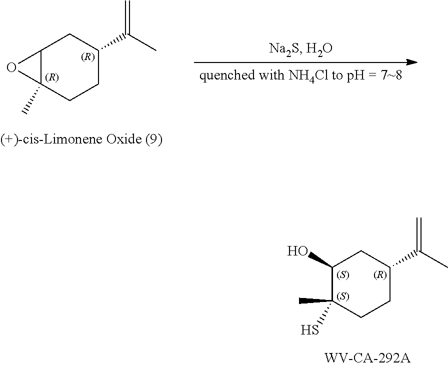

To a solution of compound (+)-cis-Limonene Oxide (9) (105 g, 689.73 mmol, 1 eq.) in H₂O (2100 mL) was added Na₂S (161.48 g, 2.07 mol, 86.82 mL, 3 eq.). The mixture was stirred at 50-80 ° C. for 48 hr. TLC (Petroleum ether: Ethyl acetate=10:1, Rf=0.24) indicated compound (+)-cis-Limonene Oxide (9) was consumed, and two new spots formed. The reaction mixture was added sat. aq. NaHCO3 until pH=7—8 at 0° C. DCM (200 mL) was added and extracted with DCM (200 mL×3), dried over Na₂SO₄, filtered and concentrated under reduced pressure to give a residue. The residue was purified by column chromatography (SiO₂, Petroleum ether/Ethyl acetate=1/0 to 1/1). Compound WV-CA-292A (76 g, 407.91 mmol, 59.14% yield) was obtained as a yellow oil. ¹FINMR (400 MHz, CHLOROFORM-d): δ =4.74 (s, 2H), 3.80 (br d, J=2.4 Hz, 1H), 2.34-2.23 (m, 1H), 2.09 (ddd, J=2.6, 11.7, 14.1 Hz, 1H), 1.99-1.87 (m, 1H), 1.75-1.50 (m, 9H), 1.44 (s, 3H). ¹³C NMR (101 MHz, CHLOROFORM-d): δ=171.20, 149.08, 109.22, 109.13, 75.42, 60.42, 48.12, 37.71, 35.22, 33.90, 29.47, 27.23, 21.03, 20.98, 14.18. GCMS: MS=186. TLC: (Petroleum ether: Ethyl acetate=10:1), R_{f}=0.24.

Preparation of compound WV-CA-292A (method II)

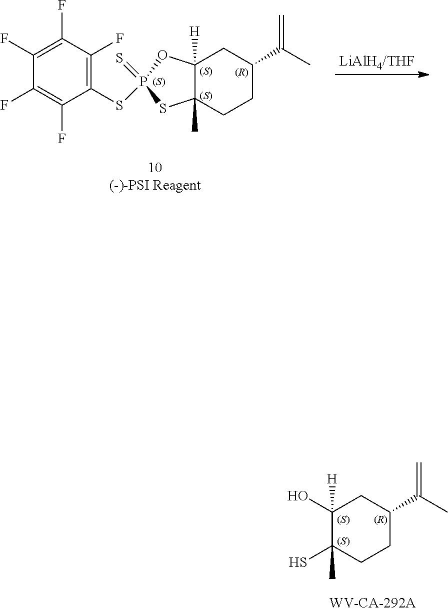

To a solution of LiA1H₄ (55 mL, 2M in THF) in THF (200 mL) cooled to -20° C. (dry ice-bath), a solution of (-)—PSI reagent (10), (50 g, 108.4 mool) in THF (300) was added drop-wise and ice-bath was removed and stirred at rt 1-1.5 h (Reaction mixture becomes slightly pinkish color). After completion of reaction (TLC monitoring) cooled to 0° C., then quenched with MeOH (2 eq. 10 mL) and solvents were evaporated to give residue, to this residue water was added and filtered with celite. The filtrate was extracted with EtOAc (500×2) and combined organic phase was dried over Na₂SO₄ and concentrated to give colorless oil, which was purified by Combiflash (220 g redsep high performance silica column) using EtOAc/Hexanes a solvent (compound eluted 30-40% of EtOAc in Hexanes). After evaporation of column fractions pooled together was dried (2 h) under vacuum to give WV-CA-292A as a colorless oil (isolated yield 81%). Analytical data was identical with Method I.

Example 19 Synthesis of WV-CA-796

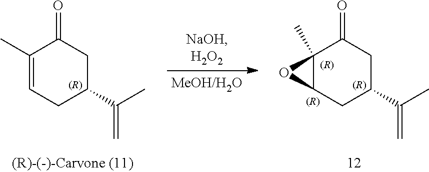

Compound (11) (30 g, 199.71 mmol, 31.28 mL, 1 eq.) was dissolved in MeOH (200 mL), H₂O₂ (65.67 g, 579.16 mmol, 55.65 mL, 30% purity, 2.9 eq.) was added, NaOH (6 M, 66.57 mL, 2 eq.) was dropped under 0° C. and the mixture was stirred at 0° C. for 3 hr. TLC (Petroleum ether/Ethyl acetate=3:1, Rf=0.43) showed compound (11) was consumed. Water (300 mL) was added and extracted with MTBE (200 mL×2), the combined organic was washed with sat. aq. Na₂SO₃ (100 mL) and sat. aq. NaHCO3, (100 mL), the organic was dried over Na₂SO₄, filtered and concentrated to get the crude. The residue was purified by column chromatography (SiO₂, Petroleum ether/Ethyl acetate=0/1 to 10/1) to get compound (12) (28.5 g, 171.46 mmol, 85.86% yield) as a colorless oil. ¹HNMR (400 MHz, CHLOROFORM-d): δ=4.79 (s, 1H), 4.72 (s, 1H), 3.45 (d, J=2.2 Hz, 1H), 2.77-2.67 (m, 1H), 2.59 (ddd, J=1.1, 4.6, 17.6 Hz, 1H), 2.37 (td, J=3.0, 14.8 Hz, 1H), 2.07-2.01 (m, 1H), 1.94-1.86 (m, 1H), 1.72 (s, 3H), 1.41 (s, 3H). TLC: (Petroleum ether/Ethyl acetate=3:1), Rf=0.43.

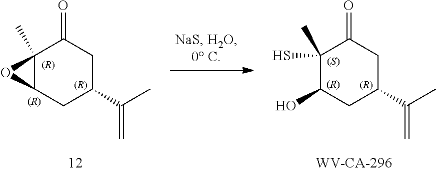

To a solution of compound (12) (9.2 g, 55.35 mmol, 1 eq) in H₂O (200 mL) was added Na₂S (12.96 g, 166.05 mmol, 6.97 mL, 3 eq). The mixture was stirred at 0° C. for 3 hr. TLC (Petroleum ether: Ethyl acetate=3:1) showed the reactant (12) was consumed and a new spot was found. The mixture was added NH₄Cl solid pH about 7-8 and extracted with DCM (100 mL×3), dried overNa₂SO₄, filtered and concentrated to give a residue, which was purified by silica gel chromatography (Petroleum ether/Ethyl acetate=1/0 to 10/1) to get WV-CA-296 (6.2 g, 30.31 mmol, 54.76% yield, 97.91% purity) as a colorless oil. TLC: (Petroleum ether/Ethyl acetate=3:1), Rf=0.32. ^{1H} NMR (400 MHz, CHLOROFORM-d): δ=4.90-4.74 (m, 2H), 4.19 (q, J=3.4 Hz, 1H), 3.16-3.03 (m, 1H), 2.94-2.81 (m, 1H), 2.44-2.35 (m, 1H), 2.30-2.14 (m, 1H), 2.13-2.04 (m, 2H), 2.00-1.90 (m, 1H), 1.83-1.75 (m, 3H), 1.57 (s, 3H). ¹³C NMR (101 MHz, CHLOROFORM-d): δ=208.55, 146.74, 110.68, 110.45, 75.06, 53.21, 41.01, 40.89, 38.76, 38.71, 36.33, 33.48, 23.72, 23.66, 20.46, 20.28. LCMS: [M+H]⁺; 201.1; LCMS purity: 97.91%. SFC: dr =98.85: 1.15.

Procedure I for Chloroderivative: In some embodiments, in an example procedure, a chiral auxiliary (174.54 mmol) was dried by azeotropic evaporation with anhydrous toluene (80 mL×3) at 35° C. in a rota-evaporator and dried under high vacuum for overnight. A solution of this dried chiral auxiliary (174.54 mmol) and 4-methylmorpholine (366.54 mmol) dissolved in anhydrous THF (200 mL) was added to an ice-cooled (isopropyl alcohol-dry ice bath) solution of trichlorophosphine (183.27 mmol) in anhydrous THF (150 mL) placed in three neck round bottomed flask through cannula under Argon (start Temp: -10.0° C., Max: temp 0° C., 28 min addition) and the reaction mixture was warmed at 15° C. for 1 hr. After that the precipitated white solid was filtered by vacuum under argon using airfree filter tube (Chemglass: Filter Tube, 24/40 Inner Joints, 80 mm OD Medium Frit, Airfree, Schlenk). The solvent was removed with rota-evaporator under argon at low temperature (25° C. ) and the crude semi-solid obtained was dried under vacuum overnight (-15 h) and was used for the next step directly.

Procedure II for Coupling: In some embodiments, in an example procedure, a nucleoside (9.11 mmol) was dried by co-evaporation with 60 mL of anhydrous toluene (60 mL×2) at 35° C. and dried under high vacuum for overnight. The dried nucleoside was dissolved in dry THF (78 mL), followed by the addition of triethylamine (63.80 mmol) and then cooled to -5° C. under Argon. The THF solution of the crude (made from general procedure I (or) II, 14.57 mmol), was added through cannula over 3 min then gradually warmed to room temperature. After 1 hr at room temperature, TLC indicated conversion of SM to product (total reaction time 1 h). Then the reaction mixture was filtered under argon using airfree filter tube, washed with THF, and dried under rotary evaporation at 26° C. to afford white crude solid product, which was dried under high vacuum overnight. The crude product was purified by ISCO-Combiflash system (rediSep high performance silica column pre-equilibrated with Acetonitrile) using Ethyl acetate/Hexane with 1% TEA as a solvent (compound eluted at 100% EtOAc/Hexanes/2% Et3N) After evaporation of column fractions pooled together, the residue was dried under high vacuum to afford the product as a white solid.

Preparation of Tsm01-Amidate (compound 500)

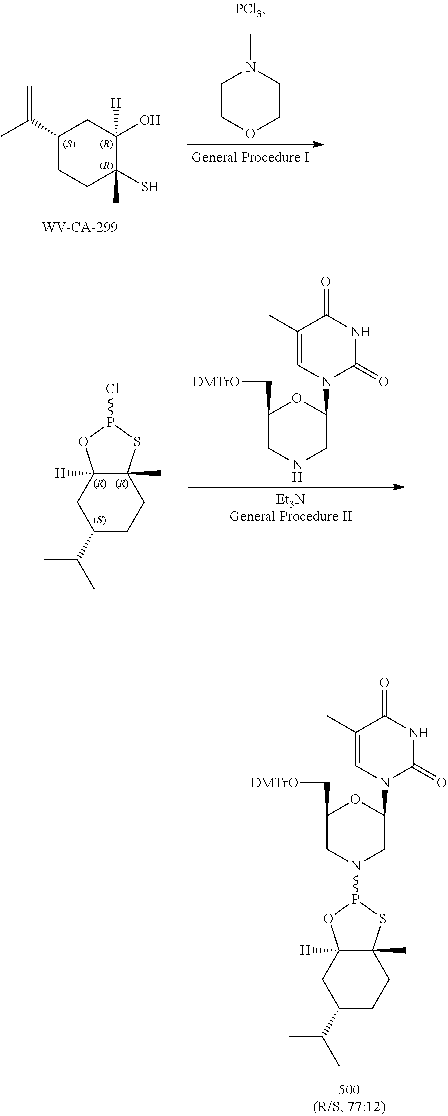

Procedure I followed by Procedure II used, off-white foamy solid, Yield: (25%). ³¹P NMR (162 MHz, CDCl₃) δ 139.47, 154.46, (ES) m/z Calculated for C₄₁H₅₀N₃O₇PS: 759.30 [M]⁺, Observed: 798.25 [M+K]⁺.

### Example 20. Synthesis of WV-DL-043R and WV-DL-0435

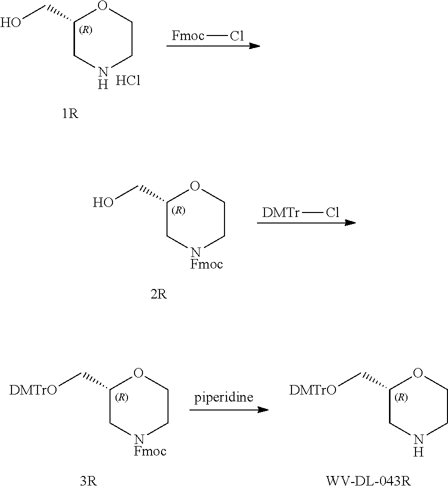

To a solution of Fmoc-Cl (47.16 g, 182.28 mmol) in dioxane (600 mL) was added Na₂CO3 (57.96 g, 546.85 mmol) in H₂O (500 mL), followed by compound 1R (28 g, 182.28 mmol) in dioxane (600 mL) added dropwise at 0-5° C., then the mixture was stirred at 5—10° C. for 12 hrs. LCMS showed desired MS was detected. TLC showed the reaction was completed, staring material was consumed. The mixture was diluted with H₂O (500 mL) and extracted with EtOAc (500 mL*4), the combined organic layers were dried over Na₂SO₄, filtered and concentrated under reduced pressure to give a residue. The residue was purified by column chromatography (Si02, Petroleum ether/Ethyl acetate=1/0 to 0/1). Compound 2R (60 g, 96.99% yield) was obtained as a white oil. LCMS: M+H⁺=340.1. TLC (Petroleum ether : Ethyl acetate=0:1) R_{f=}0.32.

Preparation of compound 3R

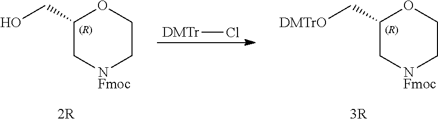

To a solution of Compound 2R (60 g, 176.79 mmol) in DCM (800 mL) and TEA (53.67 g, 530.37 mmol) was added DMT-Cl (71.88 g, 212.15 mmol) at 0° C., then the mixture was stirred at 10° C. for 12 hrs. TLC showed the reaction was completed, staring material was consumed and the product was obtained. LCMS showed the desired product was obtained. The reaction mixture was quenched by addition sat. NaHCO3 (aq., 300 mL), and then extracted with EtOAc (400 mL * 3). The combined organic layers were dried over Na₂SO₄, filtered and concentrated under reduced pressure to give a residue. The residue was purified by column chromatography (Si02, Petroleum ether/Ethyl acetate=1/0 to 0/1). Compound 3R (78 g, crude) was obtained as a red oil. TLC (Petroleum ether : Ethyl acetate=1:1) Rf=0.67. LCMS: M+H⁺=340.1.

Preparation of compound WV-DL-043R

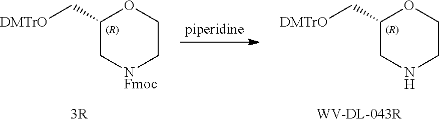

To a solution of Compound 3R (49 g, 76.35 mmol) in MeOH (125 mL) was added piperidine (97.52 g, 1.15 mol) at 15° C., then the reaction was stirred at 15° C. for12 hrs. TLC showed the reaction was completed, staring material was consumed, desired product was obtained. The crude reaction mixture was combined with another batch of the crude product (25 g scale), and concentrated under reduced pressure to give a residue. The combined crude product was purified by column chromatography (SiO₂, Petroleum ether/Ethyl acetate=1/0 to 0/1 and Ethyl acetate/Methanol=1/0 to 1/2). Finally, 31 g of product delivered. ¹H NMR (400 MHz, CHLOROFORM-d) 5=7.36 (br d, J=7.8 Hz, 2H), 7.28-7.15 (m, 7H), 6.74 (br d, J=8.8 Hz, 4H), 3.78 (br d, J=10.9 Hz, 1H), 3.70 (s, 6H), 3.65-3.58 (m, 1H), 3.53 (dt, J=2.9, 11.1 Hz, 1H), 3.10 (dd, J=5.1, 9.2 Hz, 1H), 2.98 (br d, J=12.0 Hz, 1H), 2.90 (dd, J=6.1, 9.3 Hz, 1H), 2.82-2.68 (m, 2H), 2.55 (br t, J=11.2 Hz, 1H). LCMS: purity: 98.47%. SFC: ee%, 99.68%. TLC (Ethyl acetate : Methanol=10:1) R_{f}=0.1.

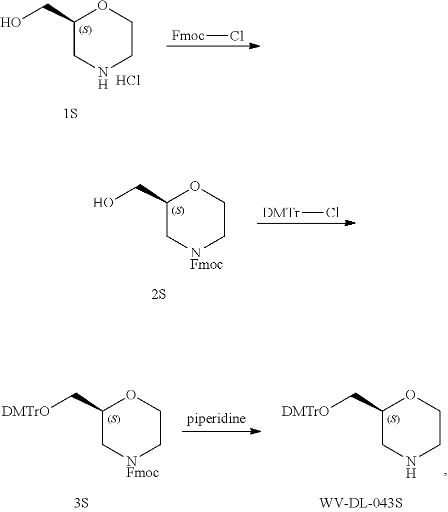

Preparation of compound 2S

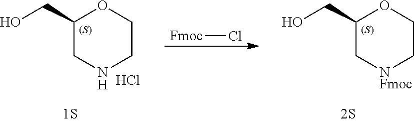

To a solution of compound 1S (25 g, 162.75 mmol) in dioxane (600 mL) was added Na₂CO3 (69.00 g, 651.01 mmol) in H₂O (500 mL), followed by Fmoc-Cl (42.10 g, 162.75 mmol) in dioxane (200 mL) added dropwised at 0-5° C., then the mixture was stirred at 5-10° C. for 12 hrs. TLC showed the reaction was completed, staring material was consumed. The desired product was obtained. The mixture was diluted with H₂O (200 mL) and extracted with EtOAc (400 mL*3), the combined organic layers were dried over Na₂SO₄, filtered and concentrated under reduced pressure to give a residue. The residue was purified by column chromatography (SiO₂, Petroleum ether/Ethyl acetate=1/0 to 0/1). Compound 2S (50 g, 147.33 mmol, 90.52% yield) was obtained as yellow oil. LCMS: M+H⁺=340.1. TLC (Petroleum ether: Ethyl acetate=0:1) Rf=0.32.

Preparation of compound 3S

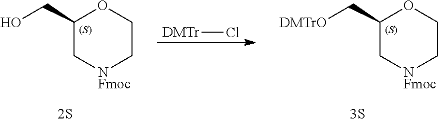

To a solution of compound 2S (50 g, 147.33 mmol) in TEA (44.72 g, 441.98 mmol) and DCM (700 mL) was added DMT-Cl (49.92 g, 147.33 mmol) at 0° C., then the mixture was stirred at 10° C. for 12 hrs. TLC showed the reaction was completed, staring material was consumed and the product was obtained. The reaction mixture was quenched by addition sat. NaHCO3 (aq., 400 mL), and then extracted with EtOAc (500 mL * 4). The combined organic layers were dried over Na₂SO₄, filtered and concentrated under reduced pressure to give a residue. The residue was purified by column chromatography (SiO₂, Petroleum ether/Ethyl acetate=1/0 to 0/1). Compound 3S (65 g, crude) was obtained as a red oil. ¹H NMR (400 MHz, CHLOROFORM-d) 5=8.35-8.22 (m, 1H), 7.66 (br s, 2H), 7.50 (br d, J=7.4 Hz, 2H), 7.37 (br d, J=7.5 Hz, 2H), 7.33-7.09 (m, 11H), 6.75 (d, J=8.6 Hz, 4H), 4.48-4.47 (m, 1H), 4.51-4.28 (m, 1H), 4.22-4.14 (m, 1H), 3.99-3.75 (m, 2H), 3.70 (s, 6H), 3.56-3.33 (m, 2H), 3.13 (br dd, J=4.7, 8.7 Hz, 1H), 3.03-2.84 (m, 2H), 2.74 (br s, 1H). TLC (Petroleum ether: Ethyl acetate=1:1) R_{f=}0.67.

Preparation of compound WV-DL -043S

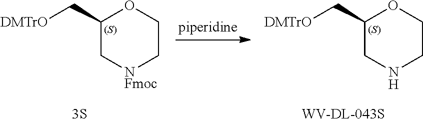

To a solution of compound 3S (65 g, 101.29 mmol) in MeOH (270 mL) was added piperidine (129.37 g, 1.52 mol) at 15° C., then the reaction was stirred at 15° C. for 12 hrs. TLC showed the reaction was completed, staring material was consumed, desired product was obtained. The reaction solution was concentrated under reduced pressure to give a residue. The residue was purified by column chromatography (Si02, Petroleum ether/Ethyl acetate=1/0 to 0/1 and EtOAc/MeOH=1/0 to 1/2) to give compound WV-DL-043S (30 g, 70.60% yield) as yellow gum. ^{1H} NMR (400 MHz, CHLOROFORM-d) 5=7.39-7.33 (m, 2H), 7.28-7.17 (m, 6H), 7.15-7.08 (m, 1H), 6.74 (d, J=8.8 Hz, 4H), 3.78 (dd, J=1.8, 11.4 Hz, 1H), 3.70 (s, 6H), 3.65-3.57 (m, 1H), 3.57-3.49 (m, 1H), 3.10 (dd, J=5.1, 9.3 Hz, 1H), 3.02-2.95 (m, 1H), 2.90 (dd, J=6.2, 9.3 Hz, 1H), 2.83-2.67 (m, 2H), 2.55 (dd, J=10.3, 12.1 Hz, 1H). ¹³C NMR (101 MHz, CHLOROFORM-d) 5=158.45, 144.93, 136.11 (d, J=5.1 Hz, 1C), 130.09, 128.86-125.78 (m, 1C), 113.08, 86.75-84.04 (m, 1C), 85.94, 76.00, 66.43 (d, J=294.2 Hz, 1C), 55.21, 49.85-48.45 (m, 1C), 47.51 (d, J=320.6 Hz, 1C), 46.47-45.26 (m, 1C). LCMS: purity: 99.44%. SFC: ee%, 97.72%. TLC (Ethyl acetate: Methanol=10:1) R_{f}=0.1.

### Example 21. Synthesis of WV-DL-045 and Various Useful Reagents

General Scheme

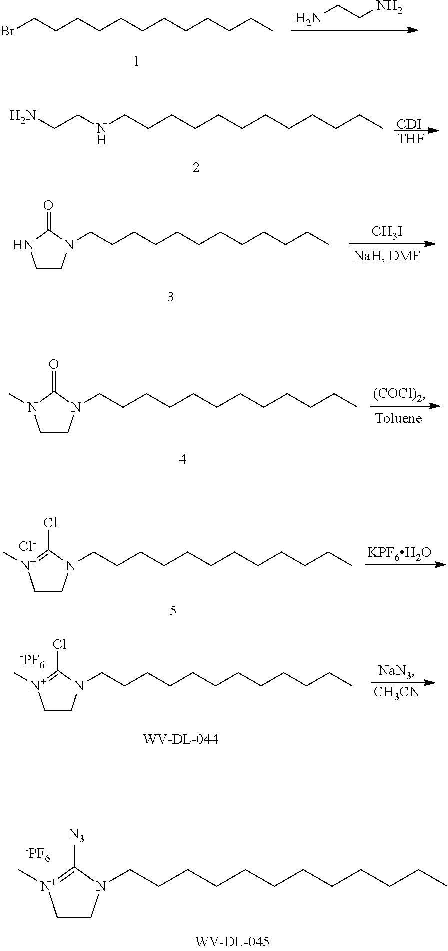

Preparation of compound 2

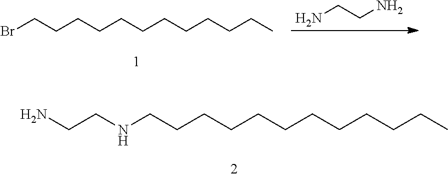

Three batches in parallel. In a one-neck round bottom flask, ethane-1,2-diamine (337.59 g, 5.62 mol) was placed with a magnetic stirring bar, and compound 1 (50 g, 200.62 mmol) was added slowly at 0° C. After finishing the addition, the reaction mixture was warmed to 25° C., and left undisturbed for an additional 1 h. 300 mL of hexane was added into the reaction mixture, which was stirred vigorously for 12 h at 25° C. LCMS showed the reaction was completed, staring material was consumed and the product was obtained, the hexane layer was decanted and dried under reduced pressure to give compound 2 (123 g) crude as colorless oil. LCMS: (M+H⁺) 229.2

Preparation of compound 3

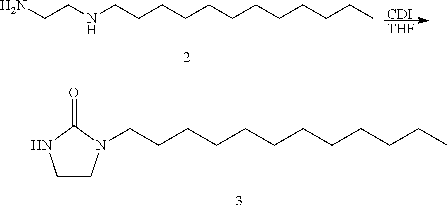

Two batches in parallel. To a solution of compound 2 (61.5 g, 269.25 mmol) and CDI (43.66 g, 269.25 mmol) in THF (630 mL) was stirred at 15° C. for 12 hr. TLC showed the reaction was completed, starting material was consumed and the product was obtained. The crude reaction mixture (126 g scale) was combined to another two batch crude product (123 g scale) and (84 g scale) for further purification. The combined crude product was purified by column chromatography on a silica gel eluted with petroleum ether: ethyl acetate (from 10/1 to 1/12) to give product 3 (95 g, 65.09% yield) as a white solid. TLC (Ethyl acetate : Methanol=10: 1) R_{f1}=0.50.

Preparation of compound 4

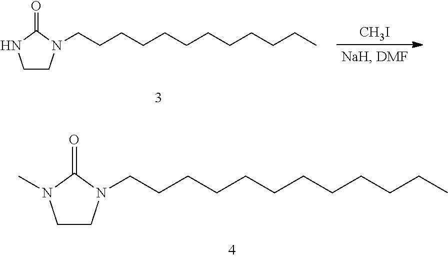

Six batches in parallel. To a solution of compound 3 (40 g, 157.23 mmol) in DMF (650 mL) was added NaH (7.55 g, 188.67 mmol, 60% purity) at 0° C. and the reaction stirred for 0.5 h, Then added CH₃I (66.95 g, 471.68 mmol) to the above reaction mixture, and stirred at 25° C. for 3 h. TLC showed the reaction was completed, starting material was consumed and the product was obtained. The reaction mixture was quenched by addition H₂O (1000 mL) at 25° C., and extracted with Ethyl acetate (1000 mL * 3). The combined organic layers were dried over anhydrous Na₂SO₄, filtered and concentrated under reduced pressure to give a residue. The residue was purified by column chromatography (Si02, Petroleum ether/Ethyl acetate=20/1 to 1/2) to give product 4 (232 g, crude) as yellow oil. ⁱH NMR (400 MHz, CHLOROFORM-d) 5=3.25-3.17 (m, 4H), 3.09 (t, J=7.3 Hz, 2H), 2.70 (d, J=1.6 Hz, 3H), 1.45-1.36 (m, 2H), 1.28-1.14 (m, 19H), 0.85-0.76 (m, 3H). TLC (Petroleum ether : Ethyl acetate=0: 1) R_{f1}=0.5.

Preparation of compound 5

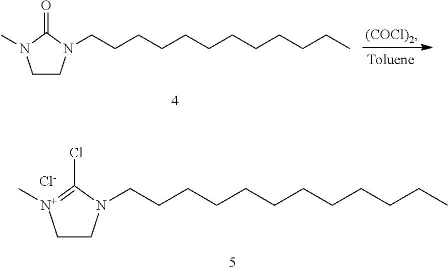

A mixture of compound 4 (30 g, 111.76 mmol, 1 eq.) in Tol.(250 mL) was degassed and purged with N₂ for 3 times, and then to the mixture was added oxalyl chloride (212.78 g, 1.68 mol, 146.75 mL, 15 eq.) and stirred at 65° C. for 72 hr under N₂ atmosphere. LCMS showed the reaction was completed, staring material was consumed, the desired product was obtained. Then the mixture was concentrated in vacuo. The white solid was washed by cooled EtOAc (100 mL*2), and then the solid was concentrated in vacuo, to give product 5 (20 g, crude) as a white solid. LCMS: M⁺, 287.3.

Preparation of compound WV-DL-044

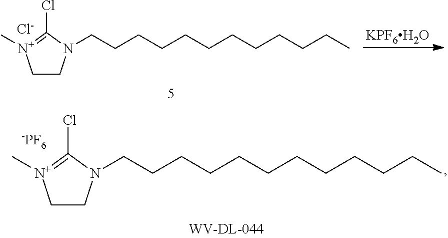

To a solution of compound 5 (8 g, 24.74 mmol) in DCM (46 mL) and H₂O (26 mL) was added potassium hexafluorophosphate (4.55 g, 24.74 mmol) at 25° C. The reaction mixture was stirred at 25° C. for 1 h. TLC showed the reaction was completed, starting material was consumed, and the desired product was obtained. The filtrate was washed with H₂O (10 mL * 2), and the white solid was desired compound. To give product WV-DL-044 (6.5 g, 60.69% yield, F6P) as a white solid. The product was combined with another two batches product (2.5 g), and (2.55 g) for analysis and delivery. Finally, 11.5 g of product was obtained. TLC (Petroleum ether : Ethyl acetate=0: 1) R_{f}=0.0.

Preparation of Lipid Azide WV-DL-045

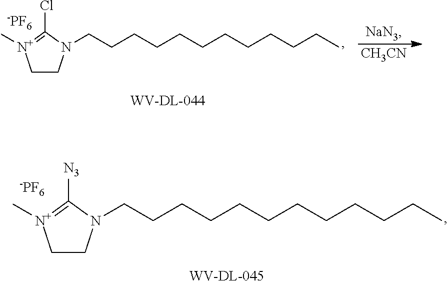

2.2 g WV-DL-044 and 495mg NaN₃ were added to a round bottom flask. Dry ACN was added forming a suspension and stirred 2.5 hr at room temperature. The reaction mixture was filtered through a pad of celite and washed with CAN. The filtrate was dried on rotovap and was then redissolved in a minimal amount ACN and the solution was precipitated with diethyl ether to afford 1.75 g of fluffy white solid. ¹14 NMR (600 MHz, Chloroform-d) δ 3.87 (dd, J=12.1, 8.1 Hz, 1H), 3.81—3.75 (m, 1H), 3.29 (t, J=7.8 Hz, 1H), 3.12 (s, 2H), 1.57—1.50 (m, 1H), 1.22 (s, 3H), 1.19 (s, 6H), 0.84—0.78 (m, 2H). ¹³C NMR (151 MHz, CDCl₃) 6154.76, 77.29, 77.07, 76.86, 49.38, 47.03, 46.52, 33.13, 31.90, 29.61, 29.61, 29.54, 29.42, 29.34, 29.05, 26.97, 26.47, 22.68, 14.11.

Synthesis of additional Azides. Various additional azide reagents were prepared utilizing suitable technologies in accordance with the present disclosure. Several examples are presented below.

### Synthesis of 2-azido-(1,3-dimethyl-3,4,5,6-tetrahydropyrimidinium) hexafluorophosphate (1d) (useful for n025)

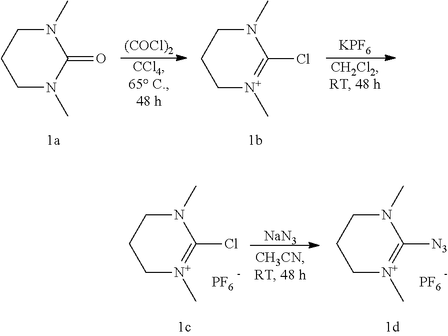

Synthesis of 2-chloro-1,3-dimethyl-3,4,5,6-tetrahydropyrimidinium chloride (1b): To 1,3-dimethyltetrahydropyrimidin-2(1H)-one, la (25.0 g, 0.195 mol, 1.0 equiv) in dry two neck round bottom flask (1 liter) under argon atmosphere was added anhydrous carbon tetrachloride (375 mL). To the reaction mixture was added freshly distilled oxalyl chloride (25.0 mL, 0.292 mol, 1.5 equiv) using additional funnel over a period of 20 min. Then reaction mixture was heated to 65° C. for 48 hrs. After completion of reaction (TLC—5% CH3OH:CH₂Cl₂; TLC charring—Phosphomolybdic acid), reaction mixture was cooled to room temperature and was added diethyl ether (300 mL). The reaction mixture was stirred at room temperature for 5 min. The obtained reaction mixture was filtered, and precipitate was washed with diethyl ether (3×500 mL). Compound was dried on high vacuum to give 2-chloro-1,3-dimethyl-3,4,5,6-tetrahydropyrimidinium chloride 1b as a brown solid (31 g, 87% yield). ¹HNMR (400 MHz, CDCl₃): δ in ppm 3.97 (t, 4H, J=5.8 Hz), 3.51 (s, 6H), 2.37-2.31 (m, 2H). MS: m/z calcd for C₆H₁₂Cl₂N₂ ([M−Cl]⁺), 147.06; found 146.95.

Synthesis of 2-chloro-1,3-dimethyl-3,4,5,6-tetrahydropyrimidinium hexafluorophosphate (1c): To 2-chloro-1,3-dimethyl-3,4,5,6-tetrahydropyrimidinium chloride, 1b (31.0 g, 0.169 mol, 1.0 equiv), in a dry round bottomed flask (1 liter) under argon atmosphere was added CH₂Cl₂ (310 mL). To the solution was added KPF6 (31.16 g, 0.169 mol, 1.0 equiv) in a portion wise over a period of 10 min. The reaction mixture was stirred at room temperature for 1.5 h. After completion of reaction (TLC—5% CH3OH:CH₂Cl₂; TLC charring—Phosphomolybdic acid), the reaction mixture was filter through celite and filter cake was washed with CH₂Cl₂ (150 mL). The filtrate was concentrated to dryness under reduced pressure to obtain crude product. The crude product was dissolved in CH₂Cl₂ (25 mL). Compound was precipitate by dropwise addition of diethyl ether. After complete precipitation, solvent was decanted to get product. Obtained was dried under vacuum to give 2-chloro-1,3-dimethyl-3,4,5,6-tetrahydropyrimidinium hexafluorophosphate (lc), as white solid (45.0 g, 91% yield). ^{1H} NMR (500 MHz, CDCl₃): δ in ppm=3.84 (s, 4H), 3.47 (s, 6H), 2.30 (s, 2H). ¹⁹F NMR (500 MHz, CDCl₃): δ in ppm=-73.02 and -74.54.

### Synthesis of 2-azido-(1,3-dimethyl-3,4,5,6-tetrahydropyrimidinium) hexafluorophosphate (1d): To 2-chloro-1,3-dimethyl-3,4,5,6-tetrahydropyrimidinium hexafluorophosphate (1c), (45.0 g, 0.154 mol, 1.0 equiv) in a dry round bottomed flask (1 liter) was added anhydrous acetonitrile (450 mL) under argon atmosphere. To the solution was added sodium azide (14.99 g, 0.231 mol, 1.5 equiv) in a portion wise over the period of 10 min. The reaction mixture stirred at room temperature for 8 hrs. After completion of reaction (TLC—5% CH3OH:CH₂Cl₂; TLC charring—ninhydrin), reaction mixture was filtered through a pad of celite and washed with CH3CN (30 mL). The obtained filtrate was dried under reduced pressure to get crude product. The crude compound was dissolved in CH3CN (150 mL). Product was precipitate by dropwise addition of diethyl ether: hexane mixture. After complete precipitation, the solvent was decanted and solid was dried under vacuum. The above precipitation procedure repeats two more times to get pure 2-azido-(1,3-dimethyl-3,4,5,6-tetrahydropyrimidinium) hexafluorophosphate ld as white solid (26 g, 57% yield). ¹H NMR (400 MHz, CDCl₃): δ in ppm=3.59 (t, 4H, J=6.0 Hz), 3.33 (s, 6H), 2.26-2.20 (m, 2H). ¹⁹F NMR (400 MHz, CDCl₃): δ in ppm=−72.99 and −74.88. MS: m/z calcd for C₆H₁₂F₆N₅P ([M−PF₆]⁺), 154.11; found 154.29. IR (KBr pellet): N₃ (2184 cm¹)

### Synthesis of 2-azido-(1,3-dimethyl-4,5,6,7-tetrahydro-1H-1,3-diazepinium) hexafluoro phosphate (2f) (useful for n026)

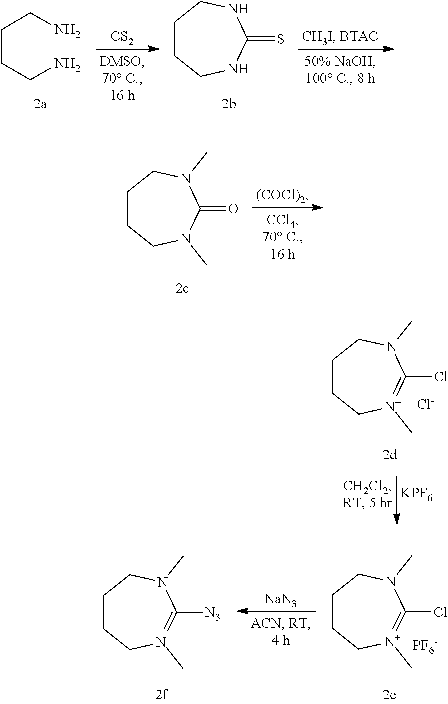

### Synthesis of 1,3-diazepane-2-thione (2b): To butane-1,4-diamine 2a (50.0 g, 0.567 mol, 1.0 equiv) in a dry round bottomed flask (1 liter) under argon atmosphere was added DMSO (500 mL). Solution was cooled 0° C. by using ice bath and carbon disulphide (41.2 mL, 0.682 mol, 1.2 equiv) was added by using addition funnel. Then, reaction mixture was heated 70° C. for 16 hrs. After completion of reaction (TLC—5% CH3OH:CH₂Cl₂), the reaction mixture was cooled to room temperature. The precipitated solid was filtered off and dried under high vacuum to get 32.0 g of product. To obtained filtrate was diluted with water (1.0 liter) and organic layer was extract with CH₂Cl₂ (3×1000 mL). The combined the organic layer dried on sodium sulphate and evaporated under reduced pressure to get crude product. This crude product was dissolved in minimum volume of CH₂Cl₂ then precipitated by dropwise addition of hexane. The precipitate was filtered and dried under high vacuum to give3-diazepane-2-thione 2b (18.0 g), as a white solid (50 9, 68% yield). ¹14 NMR (400 MHz, CDCl₃): δ in ppm=6.69 (s, 2H), 3.28-3.24 (m, 4H), 1.77-1.74 (m, 4H).

### Synthesis of 1,3-dimethyl-1,3-diazepan-2-one (2c): To 1,3-diazepane-2-thione 2b (21.0 g, 0.161 mol, 1.0 equiv) in a dry single neck round bottomed flask (250 mL) under argon atmosphere was added CH₂Cl₂ (100 mL) and solution was cooled by using ice bath. To solution was added benzyltrimethylammonium chloride (BTAC, 1.49 g, 0.008 mol, 2 mol%) followed by dropwise addition of methyl iodide (65.0 mL, 1.044 mol, 6.5 equiv) and 50% aq. NaOH solution (58.68 mL) respectively. The reaction mixture was heated to 100° C. for 8 hrs. After completion of reaction (TLC—5% CH3OH:CH₂Cl₂), the reaction mixture was cooled to room temperature. Organic layer was extracted with chloroform (3 x 1000 mL). Combined organic layer was dried over sodium sulphate and solvent was removed under reduced pressure to get crude product. The compound was purified via silica gel (100-200 mesh) column chromatography, the product was eluted with 30-80% Ethyl acetate in hexanes to give 1,3-dimethyl-1,3-diazepan-2-one, 2c as a light-yellow oil (9.00 g, 39% yield). ¹14 NMR (500 MHz, CDCl₃): δ in ppm=3.13-3.11 (m, 4H), 2.84 (s, 6H), 1.68-1.65 (m, 4H).

### Synthesis of 2-chloro-1,3-dimethyl-4,5,6,7-tetrahydro-1H-1,3-diazepinium chloride (2d): To 1,3-dimethyl-1,3-diazepan-2-one 2c, (25.0 g, 0.176 mol, 1.0 equiv) in dry two neck round bottomed flask (1 liter) under argon atmosphere was added anhydrous carbon tetrachloride (250 mL). To the solution was added freshly distilled oxalyl chloride (22.6 mL, 0.264 mol, 1.5 equiv) using addition funnel over a period of 20 min. The reaction mixture was heated to 70° C. for 16 hrs. After completion of reaction (TLC — 10% CH₃OH:CH₂Cl₂), reaction mixture was cooled to room temperature, then diluted with of diethyl ether (500 mL) and stirred for 5 min. The precipitate was collected after filtration and washed with diethyl ether (2×500 mL). Obtained crude product was dissolved in minimum amount of solvent and precipitated by addition of 50% ethyl acetate and hexanes. Compound was collected via filtration and dried under vacuum to give 2-chloro-1,3-dimethyl-4,5,6,7-tetrahydro-1H-1,3-diazepinium chloride, 2d as a white solid (30.0 g). The crude compound was directly used for next reaction without any further purification.

### Synthesis of 2-chloro-1,3-dimethyl-4,5,6,7-tetrahydro-1H-1,3-diazepinium hexafluorophosphate (2e): To 2-chloro-1,3-dimethyl-4,5,6,7-tetrahydro-1H-1,3-diazepinium chloride, 2d (30.0 g, 0.152 mol, 1.0 equiv) in dry round bottomed flak (1 liter) under argon atmosphere was added CH₂Cl₂ (300 mL). To the solution was added KPF6 (42.02 g, 0.228 mol, 1.5 equiv) in a portion wise over a period of 10 min. The reaction mixture was stirred at room temperature for 4.5 hrs. After completion of reaction (TLC—10% CH3OH:CH₂Cl₂), the reaction mixture was filter through celite and filter cake was washed with CH₂Cl₂ (150 mL), the filtrate was concentrated to dryness. The crude compound was dissolved in CH₂Cl₂ and washed with water (2×500 mL). The organic layer dried over sodium sulphate and solvent was removed under reduced pressure to give 2-chloro-1,3-dimethyl-4,5,6,7-tetrahydro-1H-1,3-diazepinium hexafluoro phosphate, 2e as white solid (25.0 g, 54% yield). ^{1H} NMR (500 MHz, CDCl₃): δ in ppm=3.90 (t, 4H, J=5.9 Hz), 3.38 (s, 6H), 2.09-2.07 (m, 4H). ¹⁹F NMR (500 MHz, CDCl₃): δ in ppm=−72.66 and −74.16.

### Synthesis of 2-azido-(1,3-dimethyl-4,5,6,7-tetrahydro-1H-1,3-diazepinium) hexafluoro phosphate (2f): To 2-chloro-1,3-dimethyl-4,5,6,7-tetrahydro-1H-1,3-diazepinium hexafluoro phosphate, 2e (25.0 g, 0.081 mol, 1.0 equiv) in a round bottomed flask (1 liter) under argon atmosphere was added anhydrous CH3CN (250 mL). To the solution was added sodium azide (7.95 g, 0.122 mol, 1.5 equiv) in a portion wise over a period of 10 min. The reaction mixture was stirred at room temperature for 4 hrs. After completion of reaction (TLC—10% CH3OH:CH₂Cl₂; TLC charring—ninhydrin), reaction mixture was filtered through a pad of celite and washed with CH3CN (30 mL). The organic layer was evaporated under reduced pressure to give crude product. The crude product was dissolved in CH3CN (50 mL) and product was precipitated by adding diethyl ether at −78° C. The solvent was removed and the solid obtained was dried under vacuum. The above precipitation procedure was repeated two times to give 2-azido-(1,3-dimethyl-4,5,6,7-tetrahydro-1H-1,3-diazepinium) hexafluoro phosphate 2f, as pale-yellow solid g (21.0 g, 82% yield). ¹HNMR (500 MHz, CDCl₃): δ in ppm=3.63 (t, 4H, J=5.5 Hz), 3.51 (d, 4H, J=25.5 Hz), 3.25 (s, 6H), 3.15 (s, 6H), 2.02-1.96 (m, 4H), 1.89 (s, 4H). ¹⁹F NMR (500 MHz, CDCl₃): δ in ppm=-72.15, -72.56, -73.67 and -74.08. MS: m/z calcd for C₆₇H₁₄F₆N₅P (IM—PF₆1⁺), 168.22; found 168.15. IR (KBr pellet): N₃ (2162 cm⁻¹).

### Synthesis of 1-azido(pyrrolidin-1-yl)methylene)pyrrolidinium)hexafluorophosphate (useful for n004)

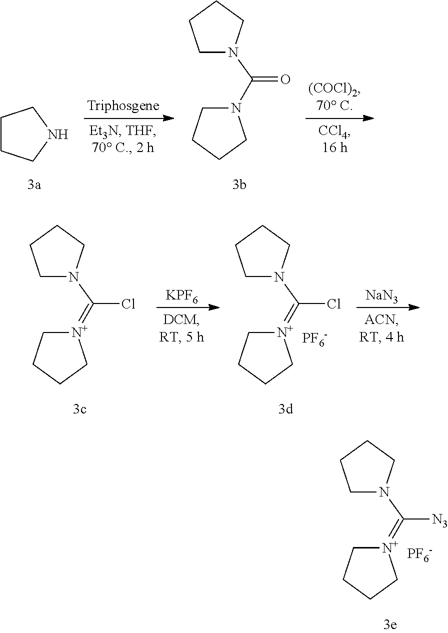

### Synthesis of di(pyrrolidin-1-yl)methanone (3b): To pyrrolidine 3a (117 mL, 1.424 mol, 1.0 equiv) in a dry three neck round bottom flask (3 liters) was added in anhydrous THF (1380 mL). To the solution was added triethylamine (212 mL, 1.521 mol, 1.1 equiv) and reaction mixture was cooled to 0° C. by using ice bath. To reaction mixture was added, a solution of triphosgene (70.0 g, 0.236 mol, 0.16 equiv, in 224 mL THF) dropwise using dropping funnel over a period of 30 min. The resulting precipitated mixture was heated at 70° C. for 2 hrs. Then reaction mixture was cooled to room temperature and stirred for another 2 hrs. TLC showed the reaction was complete (TLC—5% CH3OH:CH₂Cl₂; TLC charring—KMnO4). Then reaction mixture was filtered through Buckner funnel and Whatman filter paper. Obtained cake was washed with THF (250 mL). Filtrate was collected and solvent was removed under reduced pressure to give di(pyrrolidin-1-yl)methanone, 3b as a brown color liquid (124.0 g, 52% yield). ^{1H} NMR (500 MHz, CDCl₃): 6 in ppm=3.37 (t, 8H, J=6.9 Hz), 1.81-1.84 (m, 8H). MS: m/z calcd for C₉Hi₆N₂O ([M+H]⁺), 169.24; found 169.11.

### Synthesis of 1-(chloro(pyrrolidin-1-yl)methylene)pyrrolidinium chloride (3c): To di(pyrrolidin-1-yl)methanone 3b (124 g, 0.737 mol, 1.0 equiv) in a dry three-neck round bottom flask (3 liter) was added dry CH₂Cl₂ (1340 mL) under argon atmosphere at room temperature. To the solution was added a solution of oxalyl chloride (63.2 mL, 0.737 mol, 1.0 equiv) in dry CH₂Cl₂ (520 mL) dropwise using dropping funnel at room temperature for over a period of 40 min. Then reaction mixture was heated to 60 ° C. for 5 hrs. TLC showed the reaction was complete (TLC—5% CH3OH:CH₂Cl₂; TLC charring—KMnO4). Then the solvent was evaporated to dryness to get 1-(chloro(pyrrolidin-1-yl)methylene)pyrrolidinium chloride 3c as a brown color liquid (160.0 g). The crude material was directly used for next step.

### Synthesis of 1-(chloro(pyrrolidin-1-yl)methylene)pyrrolidinium hexafluorophosphate (3d): To 1-(chloro(pyrrolidin-1-yl)methylene) pyrrolidinium chloride 3c (160 g, 0.717 mol, 1.0 equiv) in a dry round bottom flask (2 liter) was added water (1525 mL) at room temperature. To solution, was added a saturated solution of KPF₆ (158.9 g, 0.863 mol, 1.2 equiv in 326 mL water) dropwise using dropping funnel over a period of 20 min. While adding some of product was precipitated out. Stirring was continue for another 10 min at room temperature. Then reaction mixture was filtered through Buckner funnel using Whatman filter paper. The solid was washed with water (1500 mL) and dried on high vacuum to get crude product. The crude product was dissolved in acetone (110 mL) and precipitated by dropwise addition of diethyl ether (1000 mL). The above precipitation method was repeated one more time to give 1-(chloro(pyrrolidin-1-yl) methylene)pyrrolidinium hexafluorophosphate, 3d as a cream color solid. (142.1 g, 60% yield). ^{1H} NMR (500 MHz, CDCl₃): δ in ppm=3.92 (t, 8H, J=6.2 Hz), 2.10 (t, 8H, J=6.5 Hz).

### Synthesis of 1-(azido(pyrrolidin-1-yl)methylene)pyrrolidinium hexafluorophosphate (3e): To 1-(chloro(pyrrolidin-1-yl)methylene)pyrrolidinium hexafluorophosphate, 3d (71.0 g, 0.213 mol, 1.0 equiv) in a dry round bottomed flask (500 mL) was aziotroped with acetonitrile (3×100 mL) while maintaining the bath temperature 28° C. The compound was dried on high vacuum pump for 1 hrs. To the flask was added anhydrous CH3CN (213 mL) under argon atmosphere. To the solution was added sodium azide (3.58 g, 0.055 mol) and stir for 3 hrs at 30° C. After completion of reaction (TLC—5% CH3OH:CH₂Cl₂; TLC charring—ninhydrin), reaction mixture was filtered through a pad of celite and washed with CH₃CN (50 mL). The organic layer was removed under reduced pressure to get crude product. The solid obtained was dissolved in CH3CN (60 mL) and precipitate by adding dropwise diethyl ether (850 mL). The above precipitation method was repeated one more time to geve 3e as a white color solid (65.1 g, 89% yield). ¹H NMR (400 MHz, CDCl₃): δ in ppm=3.77 (t, 8H, J=6.5 Hz), 2.03-2.06 (m, 8H). ¹⁹F NMR (400 MHz, CDCl₃): δ in ppm=−73.36 and −75.26 MS: m/z calcd for C₉H₆N₅PF₆ ([M−PF₆]⁺), 194.26; found 194.16. IR (KBr pellet): N3 (2153 cm¹).

### Synthesis of N-(azido(dimethylamino)methylene)-N-methylmethanaminium hexafluorophosphate, (2) (useful for n003)

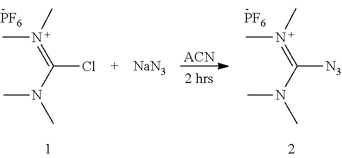

To commercially available N-(chloro(dimethylamino)methylene)-N-methylmethanaminium hexafluorophosphate(V) (1) (35.0 g, 124.7 mmol, 1.0 equiv) in a round bottom flask was added acetonitrile (100 mL). To the solution was added sodium azide (12.2 g, 187.1 mmol, 1.5 equiv). The mixture was stirred at room temperature for 1.5 hrs. After completion of reaction the reaction mixture was filtered through celite pad. The cake was washed with acetonitrile (3×40 mL). The filtrate was collected, and solvent was removed under reduced pressure to get crude product. The residue was dissolved in acetone (15 mL), then toluene was added to precipitate out product to give N-(azido(dimethylamino)methylene)-N-methylmethanaminium hexafluorophosphate, (2) as while solid (35.4 g, 99% yield). ^{1H} NMR (400 MHz, Acetonitrile-d₃) δ 3.12(s, 12H). ¹⁹F NMR (400 MHz, Acetonitrile-d3): δ in ppm=-69.57 and -70.83

### Synthesis of 4-(azido(morpholino)methylene)morpholinium hexafluorophosphate (4b) (useful for n008)

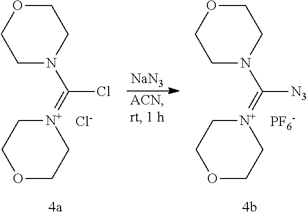

To commercially available 44chloro(morpholinium-4-ylidene)methyllmorpholine chloride 4a (41.2 g, 0.115mole, 1.0 equiv) in a round bottomed flask was added acetonitrile (115 mL). To the solution was added sodium azide (11.2 g, 0.172mole, 1.5 equiv). The mixture was stirred at room temperature for 1 hrs. After completion of reaction the reaction mixture was filtered through celite pad. The cake was washed with acetonitrile (3×40 mL). The filtrate was collected, and solvent was removed under reduced pressure to get crude product. The residue was dissolved in 1:1 toluene: acetone (160 mL) and left it in freezer overnight for formation of crystallization. The compound was collected by filtration and dried under vacuum to 4-(azido(morpholino)methylene)morpholinium hexafluorophosphate, 4b,(27 g, 64% yield). ¹HNMR (400 MHz, Acetonitrile-d₃) δ 3.86—3.71 (m, 4H), 3.65—3.58 (m, 2H), 2.34 (br.s, 8H). ¹⁹F NMR (400 MHz, Acetonitrile-d₃): δ in ppm=-71.98 and -73.80.

### Synthesis of 2-chloro-1-(4-(dimethylamino)butyl)-3-methyl-4,5-dihydro-1H-imidazol-3-ium hexafluorophosphate(WV-015A) (useful for n029)

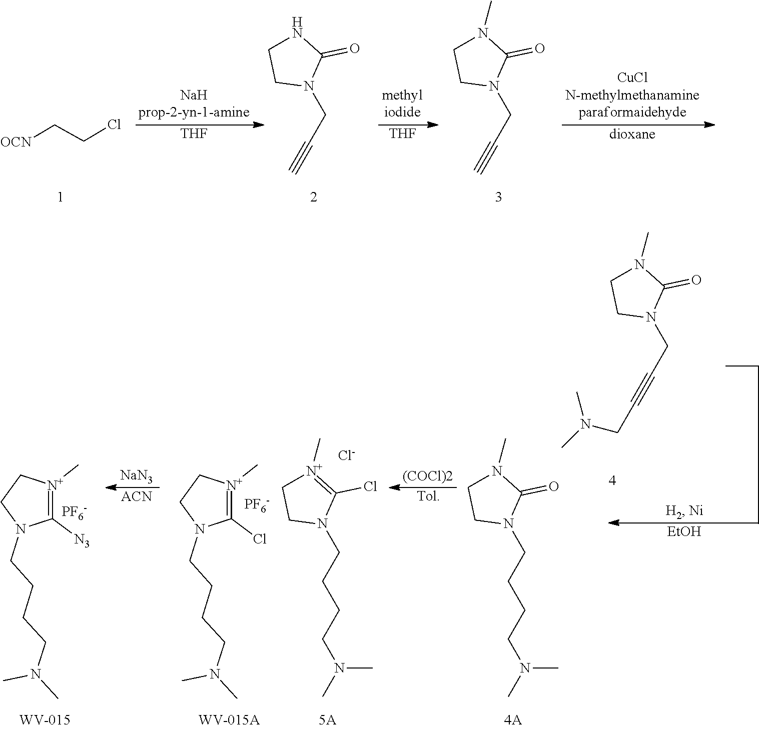

1-(prop-2-yn-1-yl)imidazolidin-2-one (2): Chloro-2-isocyanatoethane 1 (100 g, 947.66 mmol) was added at 0° C. to a stirred solution of prop-2-yn-1-amine (propargyl amine, 57.42 g, 1.04 mol, 1.0 equiv) in THF (1000 mL). The solution was warmed to 20° C. and NaH (39.80 g, 995.05 mmol, 60% purity, 0.99 equiv) was added, the mixture was stirred for 3 hr. TLC indicated prop-2-yn-1-amine was consumed completely and one new spot formed. The reaction was quenched with acetic acid (50.0 mL), the THF was removed under reduced pressure, and the residue was diluted with water 400 mL and extracted with ethyl acetate 900 mL (300 mL×3). The combined organic layers were dried over Na₂SO₄, filtered, and concentrated under reduced pressure to give a residue. The residue was purified by crystallization from ethyl acetate/hexane to give 1-(prop-2-yn-1-yl)imidazolidin-2-one (2) as a white solid (89 g, 75.65% yield).

Methyl-3-(prop-2-yn-1-yl)imidazolidin-2-one (3): To a solution of 1-(prop-2-yn-1-yl)imidazolidin-2-one (2) (89 g, 716.93 mmol, 1.0 equiv) in THF (900 mL) was added NaH (57.35 g, 1.43 mol, 60% purity, 2.0 equiv) at 0° C., 15 min later MeI (122.11 g, 860.32 mmol,) was added. The mixture was stirred at 0-20° C. for 2 hr. TLC indicated compound 2 was consumed completely and one new spot formed. The reaction mixture was quenched by addition H₂O 500 mL, and then extracted with EtOAc 1500 mL (500 mL×3). The combined organic layers were dried over Na₂SO₄, filtered, and concentrated under reduced pressure to give a residue. The residue was purified by column chromatography (SiO₂, Petroleum ether/Ethyl acetate=1/0 to 0/1) to give 1-methyl-3-(prop-2-yn-1-yl)imidazolidin-2-one (3) as a yellow oil (99 g, crude). TLC (Petroleum ether: Ethyl acetate=0:1), Rf=0.6.

1-(4-(dimethylamino)but-2-yn-1-yl)-3-methylimidazolidin-2-one (4): To a solution of 1-methyl-3-(prop-2-yn-1-ypimidazolidin-2-one (3) (99 g, 716.53 mmol, 1.0 equiv) in dioxane (1000 mL) was added CuCl (92.22 g, 931.48 mmol, 1.3 equiv), PARAFORMALDEHYDE (20 g, 2.53 mmol) and N-methylmethanamine (84.80 g, 752.35 mmol, 40% purity, 1.05 equiv). The mixture was stirred at 55° C. for 6 hr. LCMS showed the desired mass was detected. 500 g Na₂CO3 was added to the reaction mixture then stirred for 1 hr, filtered the mixture and the filtrate was concentrated under reduced pressure. The residue was purified by RP-MPLC (DAC-150 Agela C18, 450m1/min, 5-25% 40min; 25-25% 40min) to give a crude mixture. The crude was purified by column chromatography (SiO₂, Ethyl acetate/Methanol=1/0 to 5/1) to give 1-(4-(dimethylamino)but-2-yn-1-yl)-3-methylimidazolidin-2-one (4) as a yellow oil (50 g, 35.74% yield). LCMS (M+H+): 196.2 TLC (Ethyl acetate: Methanol=5:1), Rf=0.4.

1-(4-(dimethylamino)butyl)-3-methylimidazolidin-2-one (4A): A mixture of 1-(4-(dimethylamino)but-2-yn-1-yl)-3-methylimidazolidin-2-one (4) (30 g, 153.64 mmol, 1.0 equiv), Ni (10 g) in EtOH (500 mL) was degassed and purged with H2 for 3 times, and then the mixture was stirred at 80° C. for 12 hr under H2 atmosphere (15psi). LCMS showed compound 4 was consumed completely and one main peak with desired mass was detected. The mixture was filtered through celite pad and the filtrate was concentrated under reduced pressure. The residue was purified by column chromatography (SiO₂, Dichloromethane: Methanol=1/0 to 0/1) to give 1-(4-(dimethylamino)butyl)-3-methylimidazolidin-2-one (4A) as a yellow oil (30 g, crude). LCMS (M+H+): 200.3 TLC (DCM: MeOH=5:1, Rf=0.2).

chloro-1-(4-(dimethylamino)bu tyl)-3-methyl-4,5-dihydro-1H-imidazol-3-ium chloride (5A): To a solution of 1-(4-(dimethylamino)butyl)-3-methylimidazolidin-2-one (4A) (15 g, 75.27 mmol, 1.0 equiv) in toluene (50 mL) was added (COCl)₂ (191.06 g, 1.51 mol), the mixture was stirred at 65° C. for 12 hr. LCMS showed the desired mass was detected. The reaction mixture was concentrated under reduced pressure to remove solvent. The crude product was purified by re-crystallization from ACN 100 mL at 15 ° C. to give 2-chloro-1-(4-(dimethylamino)butyl)-3-methyl-4,5-dihydro-1H-imidazol-3-ium chloride (5A). as a brown solid (10 g, 52.27% yield). LCMS (M+H+): 218.3.

Chloro-1-(4-(dimethylamino)butyl)-3-methyl-4,5-dihydro-1H-imidazol-3-ium hexafluorophosphate(WV-015A). To a solution of 2-chloro-1-(4-(dimethylamino)butyl)-3-methyl-4,5-dihydro-1H-imidazol-3-ium chloride (5A) (9.75 g, 38.36 mmol, 1.0 equiv) in DCM (50 mL) and H₂O (30 mL) was added potassium;hexafluorophosphate (7.06 g, 38.36 mmol, 1.0 equiv) at 15° C. The reaction mixture was stirred at 15° C. for 1 h. A large number of solids are precipitated form the reaction mixture. The reaction mixture was filtered, and the filter cake was washed with DCM (30 mL×2), concentrated under reduced pressure to get 10 g crude. The crude was added to 200 mL H₂O, filtered, the filter cake was desired compound 2-chloro-1-(4-(dimethylamino)butyl)-3-methyl-4,5-dihydro-1H-imidazol-3-ium hexafluorophosphate (WV-015A) (8.2 g, 58.75% yield).

2-azido-1-(4-(dimethylamino)butyl)-3-methyl-4,5-dihydro-1H-imidazol-3-ium hexafluorophosphate (WV-015A). To a solution of 2-chloro-1-(4-(dimethylamino)butyl)-3-methyl-4,5-dihydro-1H-imidazol-3-ium hexafluorophosphate (WV-015A) (5.5 g, 15.1 mmol, 1.0 equiv) in dry round bottom flask (500 mL) was added dry acetonitrile (300 mL) and cooled to 0° C. To the solution was added sodium azide (1.18 g, 18.2 mmol, 1.2 equiv) and stirred for 2 hours. TLC showed completion of reaction. The reaction mixture was filtered through celite pad. Filtrate was evaporated under reduced pressure to get crude compound. MS (ESI) 371.31 (M+1)⁺.

### Synthesis of butane-1-sulfonyl azide (WLS-05) (useful for n020)

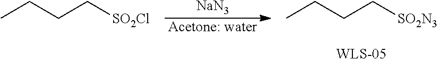

Butane-1-sulfonyl azide (WLS-05): To a solution of sodium azide (15.56 g, 0.24 mol) in water (95 mL) was added dropwise a solution of butane-1-sulfonyl chloride (25 g, 0.16 mol) in acetone (320 mL) at 0° C. for 1 h under argon atmosphere. The reaction mixture was allowed to room temperature and stirred for 3 h. After completion of reaction (monitoring by TLC), acetone was removed under reduced pressure and the reaction mixture was extracted with EtOAc (100 mL×3). The combined organic layers were dried over Na₂SO₄, filtered and concentrated under reduced pressure. Crude product was purified by silica gel column chromatography using EtOAc: hexane to afford the compound butane-1-sulfonyl azide (WLS-05) (23.53 g, 90%) as a slight brown colour oil. TLC Mobile phase details: 10% EtOAC in hexane. 1H NMR (500 MHz, CDCl₃): δ in ppm=3.32 (m, 2H, CH₂), 1.91 (m, 2H, CH₂), 1.51 (m, 2H, CH₂), 0.99 (t, J=7.3 Hz, 3H, CH₃). MS: m/z calcd for C₄H₉N₃O₂S ([M+Na]⁺), 186.18; found 186.15. IR (KBr)=2135 cm⁻¹.

### Synthesis of 6-(2,2,2-Trifluoroacetamido)hexane-1-sulfonyl azide (WLS-06) (useful for n021)

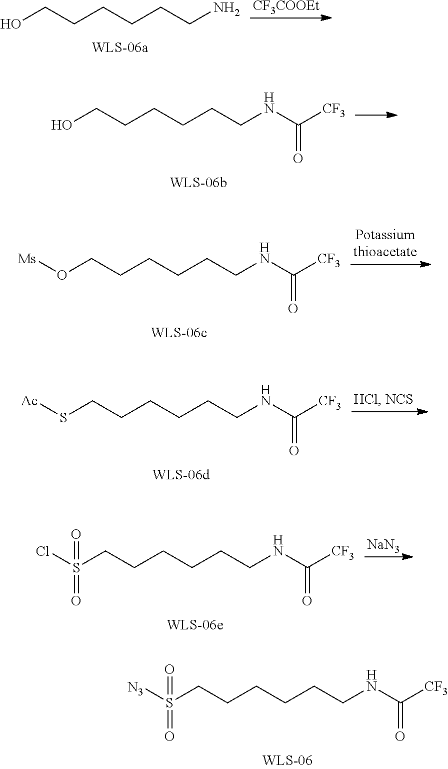

2,2,2-Trifluoro-N-(6-hydroxyhexyl)acetamide (WLS-06b): To a mixture of 6-amino hexanol (50 g, 0.43 mol) and triethylamine (148.6 mL, 1.06 mol, 2.5 equiv) in MeOH (375 mL) was cooled to 0° C. Added Trifluoroacetic anhydride (83 mL, 0.59 mol) dropwise over period of 20 min under an argon atmosphere and the reaction was allowed to warm to room-temperature and stirred 4 h, concentrated, the crude product was purified by silica gel (100-200 mesh) chromatography using EtOAc:hexane to afford the compound 2,2,2-Trifluoro-N-(6-hydroxyhexyl)acetamide (WLS-06b) (87.57 g, 96%) as a white solid. TLC Mobile phase details: 5% MeOH in DCM. ¹H NMR (500 MHz, CDCl₃): δ in ppm=6.67 (s, 1H, NH), 3.64 (t, J=6.5 Hz, 2H, CH₂), 3.36 (m, 2H, CH₂), 1.69 (s, 1H, OH), 1.59 (m, 4H, 2× CH₂), 1.39 (m, 4H, 2 x CH₂). MS: m/z calcd for C₈H₁₄F₃NO₂ ([M−H]⁺), 212.20; found 212.04.

6-(2,2,2-Trifluoroacetamido)hexyl methanesulfonate (WLS-06c): WLS-06b (50 g, 0.23 mol) was dissolved in pyridine (500 mL) under argon atmosphere. Then reaction mixture cool to 0° C. and Mesylchloride (19 mL, 0.25 mol) was added dropwise over a period of 40 min. After that, the reaction was allowed to warm to room-temperature. The solution was stirred 2 h at rt. After completion of reaction (TLC monitoring), reaction mass was quenched with water (500 mL) and extract with EtOAc (3×300 mL). The combined organic layers were dried over Na₂SO₄, filtered and concentrated under reduced pressure. The crude product was purified by silica gel (100-200 mesh) chromatography using MeOH: DCM to afford the compound WLS-06c (57.76 g, 85%) as a white solid. TLC Mobile phase details: 5% MeOH in DCM. ¹1-1 NMR (500 MHz, CDCl₃): δ in ppm=6.71 (s, 1H, NH), 4.23 (t, J=6.4 Hz, 2H, CH₂), 3.36 (m, 2H, CH₂), 3.01 (s, 3H, CH₃), 1.77 (m, 2H, CH₂), 1.61 (m, 2H, CH₂), 1.46 (m, 2H, CH₂), 1.39 (m, 2H, CH₂). MS: m/z calcd for C₉H₁₆F₃NO₄S ([M+H]⁺), 292.29; found 292.17.

S-(6-(2,2,2-Trifluoroacetamido)hexyl) ethanethioate (WLS-06d): WLS-06c (74 g, 0.254 mol) was dissolved in dry DMF (1480 mL) under argon atmosphere. Then, potassium thioacetate (58.06 g, 0.509 mol) was added in portion wise to the reaction mixture at rt (after addition formed gummy liquid, after stirring for 40 min, gummy liquid converted to clear solution). The Reaction mixture was stirred at rt for 2 h. After completion of reaction (TLC monitoring), RM was diluted with water (600 mL) and extract with diethyl ether (3×700 mL). The combined organic layers were dried over Na₂SO₄, filtered and concentrated under reduced pressure. The crude compound was purified by silica gel (100-200 mesh) chromatography using EtOAc: hexane to afford the compound WLS-06d (62.26 g, 90%) as an oil. TLC Mobile phase details: 5% MeOH in DCM. ¹H NMR (400 MHz, CDCl₃): δ in ppm=6.56 (s, 1H, NH), 3.36 (m, 2H, CH₂), 2.85 (d, J=7.3 Hz, 2H, CH₂), 2.33 (s, 3H, CH₃), 1.59 (m, 4H, 2× CH₂), 1.38 (m, 4H, 2 x CH₂). MS: m/z calcd for C₁₀H₁₆F₃NO₂S ([M−H]⁺), 270.30; found 270.17.

6-(2,2,2-trifluoroacetamido)hexane-1-sulfonyl chloride (WLS-06e): WLS-06d (24 g, 0.088 mol) was dissolved in dry MeCN (432 mL) under argon atmosphere. Then reaction mixture cool to 0° C. in ice bath. Added 2 N HCL (43.2 mL) was added dropwise over a period of 15 min and stirred for 10 min for 10 min at same temperature. Then added N-chlorosuccinimide (52.00 g, 0.390 mol) portion wise over a period of 40 min. The reaction mixture allowed to room temperature, and stirred for 2 h. After completion of reaction (TLC monitoring), the reaction mass was diluted with water (200 mL) and quench with sat. sodium bicarbonate solution at 0° C. Then, extract with diethyl ether (3×300 mL). The combined organic layers were dried over Na₂SO₄, filtered and concentrated under reduced pressure. The crude compound was purified by silica gel (100-200 mesh) chromatography using EtOAc: hexane to afford the compound WLS-06e (23.75 g, 91%). TLC Mobile phase details: 30% EtOAc in hexane. ¹HNMR (400 MHz, CDCl₃): δ in ppm=6.42 (s, 1H, NH), 3.68 (m, 2H, CH₂), 3.38 (m, 2H, CH₂), 2.06 (m, 2H, CH₂), 1.65 (m, 2H, CH₂), 1.55 (m, 2H, CH₂), 1.42 (m, 2H, CH₂). MS: m/z calcd for C₈H₁₃ClF₃NO₃S 294.70; found 294.07.

6-(2,2,2-Trifluoroacetamido)hexane-1-sulfonyl azide (WLS-06): WLS-06e (20 g, 0.078 mol) was dissolved in MeCN (295 mL) under argon atmosphere and NaN₃ (5.46 g, 0.084 mol) was added in portion wise. The reaction mixture was stirred at rt for 2 h. After completion of reaction (TLC monitoring), reaction mass was diluted with water (300 mL) and extract with ethyl acetate (3×200 mL). The combined organic layers were dried over Na₂SO₄, filtered and concentrated under reduced pressure. The crude compound was dissolved in small amount of DCM and precipitate by dropwise addition of hexane. Precipitate compound was filtered and washed with hexane to afford the white solid compound WLS-06 (18.45 g, 90%). TLC Mobile phase details: 30% EtOAc in hexane. ¹FINMR (400 MHz, CDCl₃): 6 in ppm=6.33 (s, 1H, NH), 3.36 (m, 4H, CH₂), 1.94 (m, 2H, CH₂), 1.64 (m, 2H, CH₂), 1.52 (m, 2H, CH₂), 1.42 (m, 2H, CH₂). MS: m/z calcd for C₈H₁₃F₃N₄O₃S ([M−H]⁺), 301.27; found 301.08. ¹⁹F NMR (400 MHz, CDCl₃): δ in ppm=-75.78. IR (KBr)=2147 cm¹.

### Synthesis of morpholine-4-carbonyl azide (WLS-08)

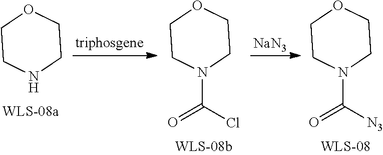 [SMILES: C1COCCN1 ; O=C(Cl)N1CCOCC1 ; [N-]=[N+]=NC(=O)N1CCOCC1 ; "NaN3"]

Morpholine-4-carbonyl chloride (WLS-08b): Triphosgene (8.57 g, 0.029 mol) was dissolved in DCM (754 mL) and cool to -5° C. using salt ice bath, then a solution of morpholine (5.0 g, 0.057 mol) and triethylamine (11.9 mL, 0.085 mol) in DCM (75 mL) was slowly added dropwise to reaction mixture over a period of 45 min. The reaction mixture was stirred for another 1 h at same temperature. After completion of reaction (TLC monitoring), reaction mixture was washed with water and extracted with DCM. The organic layers were dried over Na₂SO₄, filtered and concentrated under reduced pressure. Crude compound was purified by silica gel (100-200 mesh) chromatography using EtOAc-hexane to afford WLS-08b (2.4 g, 28%) as an oil. TLC Mobile phase details: 5% MeOH in DCM. ⁱH NMR (400 MHz, CDCl₃): 6 in ppm=3.73 (s, 6H, 3× CH₂), 3.65 (m, 2H, CH₂). MS: m/z calcd for C₅H₈C1NO₂ ([M+H]⁺), 150.57; found 149.88.

Morpholine-4-carbonyl azide (WLS-08): WLS-08b (6.7 g, 0.045 mol) was dissolved in MeCN (100 mL) under argon atmosphere and NaN₃ (3.78 g, 0.058 mol) was added at 0° C. The reaction mixture was stirred at 0° C. for 3 h. After completion of reaction (TLC monitoring), reaction mass was diluted with water (200 mL) and extract with diethyl ether (300 mL), saturated sodium carbonate (100 mL) and brine (100 mL). The organic layer was dried over sodium sulphate filtered and concentrated under reduced pressure. Crude compound was purified by silica gel (100-200 mesh) chromatography using EtOAc-hexane to afford WLS-08 (4.20 g, 60%) as an oil. TLC Mobile phase details: 5% MeOH in DCM. ¹H NMR (400 MHz, CDCl₃): δ in ppm=3.67 (m, 4H, 2× CH₂), 3.56 (m, 2H, CH₂), 3.45 (t, J=4.9 Hz, 2H, CH₂). MS: m/z calcd for C₅H₈N₄O₂ ([M+H]⁺), 157.14; found 156.80.

### Synthesis of piperidine-1-carbonyl azide (WLS-09)

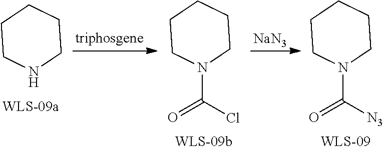 [SMILES: C1CCNCC1 ; O=C(Cl)N1CCCCC1 ; [N-]=[N+]=NC(=O)N1CCCCC1 ; "NaN3"]

Piperidine-1-carbonyl chloride (WLS-09b): Triphosgene (12.19 g, 0.041 mol) was dissolved in DCM (525 mL) and cool to -5° C. using salt ice bath, then a solution of piperidine (7.00 g, 0.082 mol) and triethylamine (22.97 mL, 0.164 mol) was slowly added dropwise to reaction mixture over a period of 45 min. The reaction mixture was stirred for another 2 h at same temperature. After completion of reaction (TLC monitoring), reaction mixture was washed with water and the organic layer was dried over sodium sulphate filtered and concentrated under reduced pressure. Crude compound WLS-09b (11.5 g) was directly used for next step. TLC Mobile phase details: 5% MeOH in DCM.

Piperidine-1-carbonyl azide (WLS-09): The crude WLS-09b (11.5 g, 0.078 mol) was dissolved in MeCN (157 mL) under argon atmosphere and NaN₃ (6.09 g, 0.094 mol) was added at 0° C. The reaction mixture was stirred at rt for 16 h. After completion of reaction (TLC monitoring), The reaction mixture was diluted with water (200 mL) and extract with diethyl ether (300 mL), saturated sodium carbonate (100 mL) and brine (100 mL). The organic layer was dried over sodium sulphate filtered and concentrated under reduced pressures. The crude compound was purified by silica gel (100-200 mesh) chromatography using EtOAc-hexane to afford WLS-09 (4.42 g, 33% in two steps) as an oil. TLC Mobile phase details: 5% MeOH in DCM. ¹H NMR (400 MHz, CDCl₃): δ in ppm=3.50 (m, 2H, CH₂), 3.36 (m, 2H, CH₂), 3.45 (t, J=4.9 Hz, 2H, CH₂), 1.59 (m, 6H, 3× CH₂). MS: m/z calcd for C₆H₁₀N₄O ([M+H]³⁰ ), 155.17; found 154.91.

### Synthesis of pyrrolidine-1-carbonyl azide (WLS-10)

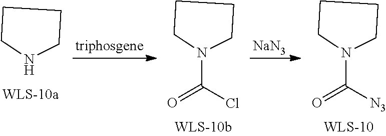 [SMILES: C1CCNC1 ; O=C(Cl)N1CCCC1 ; [N-]=[N+]=NC(=O)N1CCCC1 ; "NaN3"]

Pyrrolidine-1-carbonyl chloride (WLS-l0b): Triphosgene (12.50 g, 0.042 mol) was dissolved in DCM (450 mL) and cool to -5° C. using salt ice bath, then a solution of pyrrolidine (6.00 g, 0.084 mol) and triethylamine (23.56 mL, 0.168 mol) was added dropwise to reaction mixture over a period of 20 min. The reaction mixture was stirred for another 2 h at same temperature. After completion of reaction (TLC monitoring), reaction mass was washed with water and the organic layer was dried over sodium sulphate filtered and concentrated under reduced pressures. The crude compound WLS-10b (10.0 g) was directly used for next step. TLC Mobile phase details: 5% MeOH in DCM.

Pyrrolidine-1-carbonyl azide (WLS-10): The crude WLS-10b (10.0 g, 0.075 mol) was dissolved in MeCN (137 mL) under argon atmosphere and NaN₃ (5.84 g, 0.090 mol) was added at 0° C. The reaction mixture was stirred for 6 h. After completion of reaction (TLC monitoring), The reaction mixture was diluted with water (200 mL) and extract with diethyl ether (300 mL), saturated sodium carbonate (100 mL) and brine (100 mL). The organic layer was dried over sodium sulphate filtered and concentrated under reduced pressuress. The crude compound was purified by silica gel (100-200 mesh) chromatography using EtOAc-hexane to afford WLS-10 (6.00 g, 57% in two steps) as an oil. TLC Mobile phase details: 5% MeOH in DCM. ¹H NMR (400 MHz, CDCl₃): δ in ppm=3.45 (m, 2H, CH₂), 3.33 (m, 2H, CH₂), 1.90 (m, 4H, 2× CH₂). MS: m/z calcd for C₅H₈N₄O ([M+H]⁺), 141.15; found 140.80.

### Synthesis of 4-(2,2,2-trifluoroacetyl)piperazine-1-carbonyl azide (WLS-11)

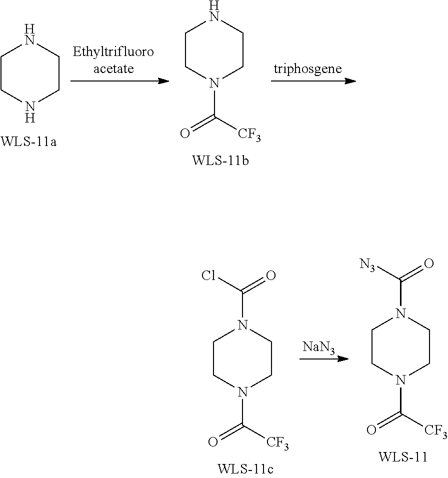 [SMILES: C1CNCCN1 ; O=C(N1CCNCC1)C(F)(F)F ; O=C(Cl)N1CCN(C(=O)C(F)(F)F)CC1 ; [N-]=[N+]=NC(=O)N1CCN(C(=O)C(F)(F)F)CC1 ; "NaN3"]

2,2,2-trifluoro-1-(piperazin-1-ypethan-1-one (WLS-11b): Ethyl trifluoroacetate (6.93 mL, 0.058 mol) was added to a suspension of piperazine (5.0 g, 0.058 mol) in THF (50 mL) at room temperature under nitrogen and stirred for 60 min and concentrated to remove solvent. The oily residue was taken up in ether and filtered and the filter cake was washed with ether. The filtrate was concentrated and purified by silica gel (100-200 mesh) column chromatography using MeOH-DCM to afford WLS-11b (6.51 g, 61%) as an oil. TLC Mobile phase details: 5% MeOH in DCM. MS: m/z calcd for C₆H₉F₃N₂O ([M+H]⁺), 183.15; found 182.65.

4-(2,2,2-Trifluoroacetyl)piperazine-1-carbonyl chloride (WLS-11c): Triphosgene (5.29 g, 0.018 mol) was dissolved in DCM (487 mL) and cool to -5° C. using salt ice bath, then a solution of WLS-11b (6.50 g, 0.036 mol) and triethylamine (9.97 mL, 0.071 mol) was slowly added dropwise to reaction mixture over a period of 20 min. The reaction mixture was stirred for another 1 h at same temperature. After completion of reaction (TLC monitoring), reaction mass was washed with water and organic layer was dried over sodium sulphate and concentrated under reduced pressures. Crude compound WLS-11c (8.1 g) was directly used for next step. TLC Mobile phase details: 5% MeOH in DCM.

4-(2,2,2-trifluoroacetyl)pipe razine -1-carbonyl azide (WLS-11): The crude WLS-11c (8.1 g, 0.033 mol, 1.0 equiv) was dissolved in MeCN (111 mL) under argon atmosphere and NaN₃ (2.58 g, 0.040 mol,) was added at 0° C. The reaction mixture was stirred for 2 h. After completion of reaction (TLC monitoring), The reaction mixture was diluted with water (200 mL) and extract with diethyl ether (300 mL), saturated sodium carbonate (100 mL) and brine (100 mL). The organic layer dried over sodium sulphate, filtered and concentrated under reduced pressures. Crude compound was purified by silica gel (100-200 mesh) chromatography using EtOAc-hexane to afford WLS-11 (6.31 g, 70% in two steps) as an oil. TLC Mobile phase details: 5% MeOH in DCM. ¹H NMR (400 MHz, CDCl₃): δ in ppm=3.65 (m, 6H, 2× CH₂), 3.55 (d, J=2.5 Hz, 2H, CH₂). MS: m/z calcd for C₇H₈F₃N₅O₂ ([M+H]⁺), 252.17; found 252.00.

### Synthesis of 4-Methylpiperazine-1-carbonyl azide (WLS-12)

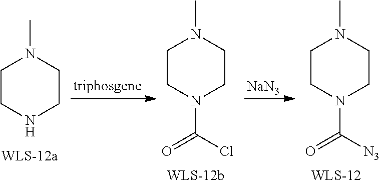

Methylpiperazine-1-carbonyl chloride (WLS-12b): Triphosgene (7.40 g, 0.025 mol) was dissolved in CH₂Cl₂ (750 mL) and cool to −5° C. using salt ice bath, then a solution of N-methylpiperazine (5.00 g, 0.050 mol) and diisopropylethylaine (17.38 mL, 0.100 mol) in CH₂Cl₂ (150 mL) was slowly added dropwise to reaction mixture over a period of 30 min. The reaction mixture was stirred for another 2 h at same temperature. After completion of reaction (TLC monitoring), RM was washed with water and organic layer was dried over sodium sulphate, filtered and concentrated under reduced pressures. Crude compound WLS-12b (8.0 g) was directly used for next step. TLC Mobile phase details: 5% MeOH in DCM.

Methylpiperazine-1-carbonyl azide (WLS-12): The crude WLS-12b (8.0 g, 0.049 mol) was dissolved in MeCN (112 mL) under argon atmosphere and NaN₃ (3.83 g, 0.059 mol) was added at 0° C. Then reaction mixture was stirred for 3 h. After completion of reaction (TLC monitoring), The reaction mixture was diluted with water (200 mL) and extract with diethyl ether (300 mL), saturated sodium carbonate (100 mL) and brine (100 mL). The organic layer dried over sodium sulphate, filtered and concentrated under reduced pressures. Crude compound was purified by silica gel (100-200 mesh) chromatography using EtOAc-hexane to afford WLS-12 (3.60 g, 43% in two steps) as an oil. TLC Mobile phase details: 5% MeOH in DCM. ¹14 NMR (400 MHz, CDCl₃): δ in ppm=3.58 (t, J=5.1 Hz, 2H, CH₂), 3.46 (t, J=5.1 Hz, 2H, CH₂), 2.38 (m, 4H, 2× CH₂), 2.30 (s, 3H, CH₃). MS: m/z calcd for C₆H₁₁N₅O ([M+H]⁺), 170.19; found 169.81.

### Synthesis of 4-(6-(2,2,2-Trifluoroacetamido)hexanoyl)piperazine-1-carbonyl azide (WLS-13)

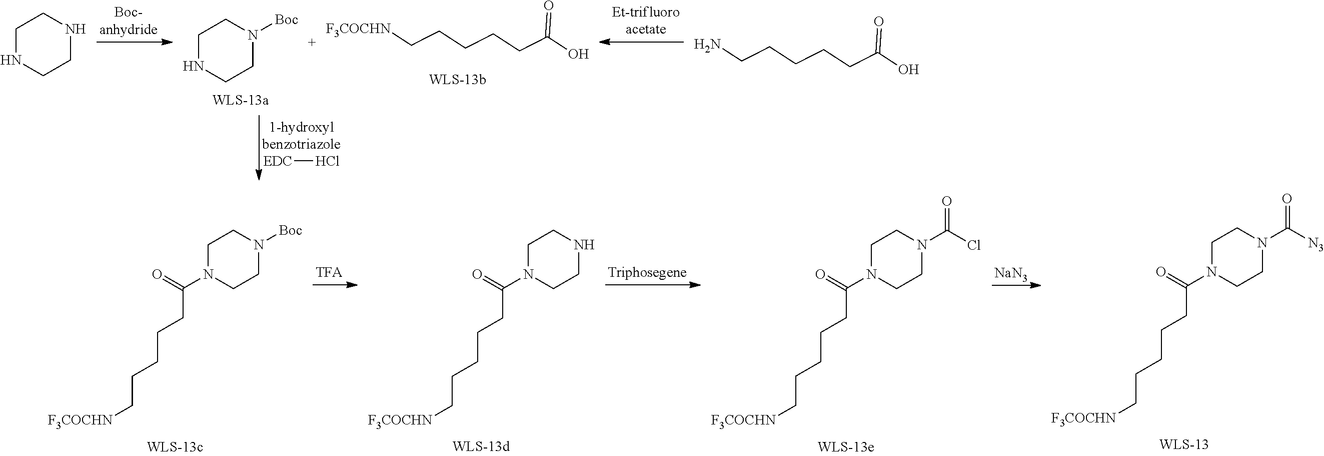

N-(tert-Butoxycarbonyl)-piperazine (WLS-13a: 1-Boc-piperazine): Piperazine (12 g, 139.3 mmol) was dissolved in dry CH₂Cl₂ (240 mL) and the solution was cooled to 0° C. To the reaction mixture, solution of di-teat-butyl dicarbonate (Boc20) (15.2 g, 69.64 mmol) in dry CH₂Cl₂ (160 mL) was added dropwise (over period of 20 min). Then reaction mixture stirred at rt for 24 h. After completion of reaction, precipitate formed was filtered off and washed with CH₂Cl₂ (2×40 mL), and the combined filtrate was separated and washed with H₂O (3×80 mL), brine (60 mL), dried over Na₂SO₄, filtered and concentrated under reduced pressures. Crude product was purified by silica gel column chromatography using CH₂Cl_{2:}MeOH to afford the compound WLS-13a (11.6 g, 45%) as a white solid. TLC Mobile phase details: 20% MeOH in DCM. ¹H NMR (500 MHz, CDCl₃): δ in ppm=3.32 (t, J=4.8 Hz, 4H, 2× CH₂,), 2.74 (t, J=4.5 Hz, 3H, 2× CH₂), 1.68 (s, 1H, NH), 1.40 (s, 9H, 3× CH₃). MS: m/z calcd for C₉H₁₉N₂O₂ ([M+H]⁺), 187.25; found 187.04.

6-(2,2,2-Trifluoroacetamido)hexanoic acid (WLS-13b): A solution of 6-amino hexanoic acid (21 g, 0.160 mol) and triethylamine (22.4 mL, 0.160 mol) in MeOH (80 mL) was cooled to 0° C. Trifluoroacetic anhydride (24 mL, 0.192 mol) was added dropwise over period of 20 min under an argon atmosphere and the reaction was allowed to room-temperature and stirred 16 h. After completion of reaction, solvent was evaporated. The crude compound was cool to 0° C., 2 N HCl (400 mL) was added dropwise. After addition precipitate compound was filtered to get white compound. To take out rest compound from filtrate, filtrate is solution is saturated with NaCl and extracted with diethyl ether (2×200 mL). The solid compound is also dissolved in diethyl ether (200 mL) and washed with water (2×200 mL). The combined organic layer (from solid and from filtrate) was dried over sodium sulphate and evaporated. The crude compound was dissolve in small amount of diethyl ether and precipitate by adding dropwise hexane. Precipitate compound was filtered and wash with hexane to afford the compound WLS-13b (33.0 g, 91%) as a white solid. TLC Mobile phase details: 10% MeOH in DCM. ⁱH NMR (500 MHz, CDCl₃): δ in ppm=12.00 (s, 1H, COOH), 9.39 (s, 1H, NH), 3.17 (dd, J=13.1, 6.9 Hz, 2H, CH₂), 2.20 (t, J=7.6 Hz, 2H, CH₂), 1.50 (m, 4H, 2× CH₂), 1.26 (m, 2H, CH₂). MS: m/z calcd for C₈H₁₂F₃NO₃ ([M−H]⁺), 226.18; found 226.02.

(Tert-butyl 4-(6-(2,2,2-trifluoroacetamido)hexanoyl)piperazine-1-carboxylate (WLS-13c).

To a solution of WLS-13b (15.00 g, 0.066 mol) and 1-hydroxybenztriazole (9.72 g, 0.072 mol) in anhydrous methylene chloride (375 mL) was added ethyl 3-(dimethylamino)propyl carbodiimide, hydrochloride salt (13.8 g, 0.072) at 0° C. under argon atmosphere. The mixture was stirred for 30 minutes at 0° C. Then WLS-13a (12.3 g, 0.066 mol) and diisopropylethylamine (13.8 mL, 0.793 mol) were added and the mixture became a homogeneous solution. The reaction mixture was stirred for 3 h at 0° C. The solution was slowly warmed to room temperature and stir for another 2 h at rt. After completion of reaction (TL^{s} monitoring), RM cool to 0° C. and quench with ice cold water (400 mL). The separate organic layer wash with 5% sodium bicarbonate solution. (2×500 mL). The combined organic layer dried on sodium sulphate, filtered and concentrated under reduced pressures. The crude product was dissolve in small amount of CH₂Cl₂ and precipitate by adding dropwise hexane. Precipitate compound was filtrate and wash with hexane to afford the compound WLS-13c (33.0 g, 91%) as a white solid. TLC Mobile phase details: 5% MeOH in DCM. ¹H NMR (500 MHz, CDCl₃): δ in ppm=9.38 (s, 1H, NH), 3.41 (m, 2H, CH₂), 3.26 (s, 2H, CH₂), 3.16 (dd, J=13.0, 6.8 Hz, 3H, CH, CH₂), 2.30 (t, J=7.5 Hz, 2H, CH₂), 1.48 (m, 5H, CH, 2× CH₂), 1.40 (s, 9H, 3× CH₃), 1.28 (m, 4H, 2× CH₂). MS: m/z calcd for C₁₇H₂₈F₃N₃O₄ ([M−H]⁺), 394.42; found 394.33.

2,2,2-Trifluoro-N-(6-oxo-6-(piperazin-1-yl)hexyl)acetamide (WLS-13d) : WL S-13c (18.30 g, 0.046 mol) was dissolved in CH₂Cl₂ (725 mL) and cool to 0° C. under argon atmosphere. Then TFA:CH₂Cl₂(1;1, 181.3 mL) solution was added dropwise over period of 45 min at 0° C. After that, reaction mixture allowed to rt and stirred for 4 h. After completion of reaction (TLC monitoring), solvent was evaporated to dryness using base trap to get crude compound. The crude compound was dissolved in 15% MeOH:CH₂Cl₂ (100 mL) and cool to 0° C. and quench with saturated sodium bicarbonate solution (pH up to neutral). Then 400 mL water was added and extract with 15% MeOH:CH₂Cl₂ (6×300 mL, extract up to there was no product in aqueous layer). The combined organic layer was dried on sodium sulphate, filtered and concentrated under reduced pressures to get crude WLS-13d (12.82 g) as an oil. Crude compound was directly used for next reaction. TLC Mobile phase details: 10% MeOH in DCM. MS: m/z calcd for C₁₂H₂₀F₃N₃O₂ ([M−H]⁺), 294.31; found 294.17.

4-(6-(2,2,2-trifluoroacetamido)hexanoyl)piperazine-1-carbonyl chloride (WLS-13e): To a solution of WLS-13d (12.2 g, 0.041 mol) and diisopropylethylamine (29.0 mL, 0.166 mol) in anhydrous THF (610 mL) was added dropwise triphsogene (6.13 g, 0.021) solution in THF (190 mL) over a period of 30 min at 0° C. under argon atmosphere. The reaction mixture stirred at same temperature for another 30 min. The reaction mixture allowed rt and stirred for another 3 h. After completion of reaction (TLC monitoring), reaction mass was filtered and solid was washed with THF. The filtrate was evaporated to dryness. The crude product was dissolved in CH₂Cl₂ (300 mL) and washed with water (2×300 mL). The combined organic layer dried on sodium sulphate filtered and concentrated under reduced pressures. The crude compound was purified by silica gel (100-200 mesh) chromatography using hexane: ethyl acetate to afford WLS-13e (6.5 g, 33% in two steps) as a slight yellow solid. TLC Mobile phase details: 10% MeOH in DCM. ¹H NMR (500 MHz, CDCl₃): δ in ppm=7.27 (s, 1H, NH), 3.71 (m, 6H, 3× CH₂), 3.58 (d, J=15.9 Hz, 2H, CH₂), 3.49 (m, H, CH), 3.39 (m, 2H, CH₂), 2.37 (t, 2H, J=7.1 Hz, CH₂), 1.65 (m, 4H, 2× CH₂), 1.39 (m, 2H, CH₂). MS: m/z calcd for C₁₃H₁₉ClF₃N₃O₃ ([M−H]⁺), 356.76; found 355.98.

4-(6-(2,2,2-Trifluoroacetamido)hexanoyl)piperazine-1-carbonyl azide (WLS-13): To a solution of sodium azide (1.31 g, 0.020 mol) in water (8.2 mL) was added dropwise over 20 min a solution of WLS-13e (6 g, 0.017 mol) in acetone (22.2 mL) at 0° C. under argon atmosphere. The reaction mixture was allowed to room temperature and stirred for 3 h. After completion of reaction (TLC monitoring), acetone was removed under reduced pressure. Then water was added (100 mL) and extracted with EtOAc (80 mL×3). The combined organic layers were dried over Na₂SO₄ and solvent was removed under reduced pressure. The crude compound was purified by silica gel column chromatography using EtOAc: hexane to afford the compound WLS-13 (2.01 g, 33%) as a slight brown colour oil. TLC Mobile phase details: 5% MeOH in DCM. ¹H NMR (500 MHz, CDCl₃): δ in ppm=7.00 (s, 1H, NH), 3.60 (m, 4H, 3× CH₂), 3.47 (t, J=7.2 Hz, 4H, 2× CH₂), 3.41 (m, 2H, CH₂), 2.36 (t, J=6.2 Hz, 2H, CH₂), 1.65 (m, 4H, 2× CH₂), 1.39 (m, 2H, CH₂). MS: m/z calcd for C₁₃H₁₉F₃N₆O₃ ([M−H]⁺), 363.33; found 355.98.

2-azido-1-butyl-3-methyl-4,5-dihydro-1H-imidazol-3-ium hexafluoro-phosphate (V) (WLS-43)

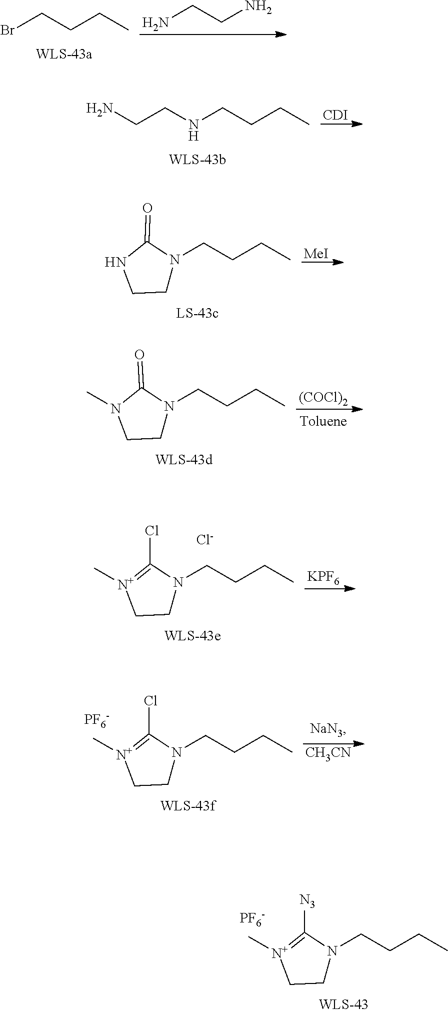

Preparation of compound WLS-43b: In a clean and dry three-neck 3 Lit round bottom flask, ethane-1,2-diamine (1000 mL, 14.975 mol, 25.65 equiv) was placed with a magnetic stirring bar, and compound WLS-43a (80 g, 0.584 mol, 1.0 equiv) was added dropwise at 0° C. by using addition funnel. After finishing the addition, the reaction mixture was warmed to 25° C., and left undisturbed for an additional 1 h. Then, 600 mL of hexane was added into the reaction mixture and stirred vigorously for 16 h at 25° C. TLC showed the reaction was completed, staring material was consumed and the new spot was formed (TLC-10% MeOH:EtOAc; TLC charring—Phosphomolybdic acid). The hexane layer was separated by using separatory funnel, dried over sodium sulphate and evaporated to dryness under reduced pressure to get compound WLS-43b (44.0 g) as a crude colorless oil. Crude compound was directly used for next step without any further purification. MS: m/z calcd for C₆H₁₆N₂ ([M+H]⁺), 117.21; found 117.15.

Preparation of compound WLS-43c: WLS-43b (44.0 g, 0.379 mol, 1.0 equiv), was taken in clean and dry 1 Lit two neck RBF under argon atmosphere. Then add 440 mL of THF to RBF. Cool the RB in ice bath (0° C. ). Add portion wise 1,1′-Carbonyldiimidazole (63.24 g, 0.390 mol, 1.03 equiv) to reaction mixture for period of 10 min. The reaction mixture was stir at 15° C. for 12 h. TLC showed the reaction was completed, staring material was consumed and the product was formed (TLC—10% MeOH:EtOAc; TLC charring—Phosphomolybdic acid). After completion of reaction, solvent was dried and purified on silica gel column chromatography (100-200 mesh). The product was eluted with 80% ethyl acetate: hexane to EtOAc. Fraction containing product was evaporated to get 35.02 g (65% yield) of WLS-43c as a colorless oil. ¹H NMR (500 MHz, CDCl₃): δ in ppm=4.77 (s, 1H), 3.45-3.48 (m, 4H), 3.18 (t, 2H, J=7.6 Hz), 1.52-1.46 (m, 2H), 1.34 (td, 2H, J=15.0 Hz, 7.3 Hz), 0.93 (t, 3H, J=7.6 Hz). MS: m/z calcd for C₇F₁₁₄N₂O ([M+H]⁺), 143.20; found 143.46.

Preparation of compound WLS-43d: WLS-43c (30.0 g, 0.211 mol, 1.0 equiv) was taken in clean and dry 2 Lit three neck RBF under argon atmosphere. Then, add 450 mL of dry DMF to RBF containing starting material. Cool the reaction mixture in ice bath (Temp. 0° C. ). Then, add portion wise 60% NaH (10.14 g, 0.253 mol) to reaction mixture for period of 20 min. at 0° C. and stir 40 min at same temp. Then add dropwise methyl iodide (39.4 mL, 0.633 mol) to the reaction mixture at 0° C. for duration of 15 min. Then allow the reaction mixture to room temprature and stir for 2 h. TLC showed the reaction was completed, staring material was consumed and the new spot was formed (TLC-EtOAc; TLC charring—Phosphomolybdic acid). After completion of reaction, reaction mixture was cool to ° C. in ice bath and quenched with ice cold water (1 Lit). Then extracted with ethyl acetate 2×800 mL). The organic layer was washed with ice cold water (2×1000 mL) and dried over sodium sulfate, filtered and concentrated to dryness. The crude product was purified by silica gel column chromatography (100-200 mesh). The product was eluted with 10%-40% ethyl acetate:hexane. The fraction containing product was evaporated to get 18.0 g (55% yield) of WLS-43d as a white colour solid. ¹14 NMR (400 MHz, CDCl₃): δ in ppm=3.28 (s, 4H), 3.18 (t, 2H, J=7.3 Hz), 2.78 (s, 3H), 1.51-1.44 (m, 2H), 1.38-1.30 (m, 2H), 0.93 (t, 3H, J=7.3 Hz). MS: m/z calcd for C₈H₁₆N₂O ([M+H]⁺), 157.23; found 157.48.

Preparation of compound WLS-43e: WLS-43d (30.0 g, 0.192 mol, 1.0 equiv) was taken in clean and dry 1 Lit single neck round bottom flask under argon atmosphere. Then add 300 mL of dry toluene to RBF containing starting material under argon atmosphere. After that add dropwise oxalyl chloride (247.0 mL, 2.880 mol) using addition funnel for a period of 30 min at rt. Then, reaction mixture was heated to 65° C. for 72 hrs. After completion of reaction (TLC—5% MeOH:DCM; TLC charring—Phosphomolybdic acid) solvent was evaporated to dryness to get crude compound. The crude compound was co-evaporate with toluene (200 mL) and washed with cold ethyl acetate:hexane (70:30, 2×1000 mL), diethyl ether:hexane (20:80, 1000 mL) and dried to get 34.0 g of crude WLS-43e as brown colour semi solid. Crude compound was directly used for next step without any further purification. MS: m/z calcd for C₈H₁₆Cl₂N₂ ([M−Cl]⁺), 175.68; found 176.89.

Preparation of compound WLS-43f: WLS-43e (29.0 g, 0.137 mol, 1.0 equiv) was taken in clean and dry 1 L single neck round bottom flask and dissolved in 290 mL DCM under argon atmosphere. Then added aq solution of KPF₆ (25.28 g, 0.137 mol, in 188 mL of water). Stir the reaction mixture at rt for 2 h. After completion of reaction (TLC—5% MeOH:DCM), the reaction mixture was poured into ice water, and extracted with DCM (2×400 mL). The combined organic layer washed with water (400 mL) and dried over sodium sulphate, filtered and evaporated to dryness. Then, residue was dissolved in DCM and product was precipitate by dropwise addition of diethyl ether under stirring. The solvent was decant and solid was dried under high vacuum. The above precipitation procedure repeat two more times to get 35.0 g (80% yield) of WLS-43f as a white solid. ¹H NMR (500 MHz, CDCl₃): δ in ppm=4.14-4.04 (m, 4H), 3.53 (t, 2H, J=7.6 Hz), 3.23 (s, 3H), 1.67-1.61 (m, 2H), 1.41-1.35 (m, 2H), 0.96 (t, 3H, J=7.2 Hz). ¹⁹F NMR (400 MHz, CDCl₃): δ in ppm=−73.18 and −74.70.

Preparation of compound WLS-43: WLS-43f (39.5 g, 0.123 mol, 1.0 equiv) was taken in clean and dry 1 L single neck round bottom flask and dissolved in 200 mL of Dry MeCN under argon atmosphere. Then, added portion wise sodium azide (12.01 g, 0.185 mol, 1.5 equiv) to the RM and stir at rt for 4 h. After completion of reaction (TLC—5% MeOH:DCM; TLC charring—ninhydrin), reaction mixture was filtered through a pad of celite and washed with MeCN (20 mL). The organic layer was evaporated to dryness. The crude compound was dissolve in minimum amount of MeCN and precipitate by adding dropwise diethylether (500 mL) at −78. The above precipitation procedure repeat two more times to get 38.0 g (94% yield) of WLS-43 as a light yellow solid. ¹H NMR (500 MHz, CDCl₃): δ in ppm=3.98-3.94 (m, 2H), 3.89-3.85 (m, 2H), 3.40 (t, 2H, J=7.6 Hz), 3.20 (s, 3H), 1.64-1.59 (m, 2H), 1.35 (td, 2H, J=15.0 Hz, J=7.3 Hz), 0.95 (t, 3H, J=7.6 Hz). ¹H NMR (500 MHz, CDCl₃): δ in ppm=−73.49 and −75.01. MS: m/z calcd for C₈H₁₆F₆N₅P ([M−PF₆]⁺), 182.25; found 182.17. IR (KBr pellet): N₃ (2174 cm⁻¹).

### 2-azido-1,3-dibutyl-4,5-dihydro-1H-imidazol-3-ium hexafluorophosphate (V) (WLS-44)

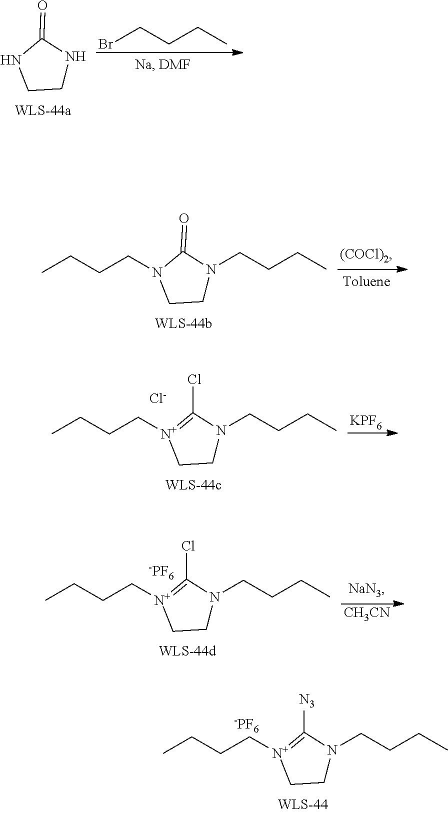

Preparation of compound WLS-44b: In a clean and dry two-neck 500 mL round bottom flask, WLS-44a (20.0 g, 0.232 mol, 1.0 equiv) was placed with a magnetic stirring bar and dissolved by adding DMF (200 mL). Then cool the RBF to 0° C. by using ice bath. After that, add sodium hydride (18.58 g, 0.465 mol, 2.0 equiv) portion wise for period of 40 min at 0° C. Stir the reaction mixture at 0° C. for 30 min. Then added bromo butane (100 mL, 0.927 mol, 4.0 equiv) dropwise by using addition funnel for period of 20 min at 0° C. and stir for 2 h. TLC showed the reaction was completed, staring material was consumed and the product was formed (TLC—30% EtOAc;Hexane, TLC charring—Phosphomolybdic acid). After completion of reaction, reaction mixture poured into ice and extracted with ethyl acetate (100 mL×2) and organic layer washed with ice cold water (1000 mL×2). Organic layer dried over sodium sulphate, filtered and evaporated to dryness to get crude compound. The crude product was purified by silica gel column chromatography (100-200 mesh). The product was eluted with 15%-30% ethyl acetate:hexane. The fraction containing product was evaporated to get 40.0 g (87% yield) of WLS-44b as a yellow liquid. ¹H NMR (400 MHz, CDCl₃): δ in ppm=3.27 (s, 4H), 3.17 (t, 4H, J=7.4 Hz), 1.44-1.51 (m, 4H), 1.33 (dt, 4H, J=22.5 Hz, 7.2 Hz) 0.93 (t, 6H, J=7.4 Hz).

Preparation of compound WLS-44c: WLS-44b (40.0 g, 0.202 mol, 1.0 equiv) was taken in clean and dry 1 Lit two neck RBF under argon atmosphere. Then add 400 mL of dry toluene to RBF containing SM under argon atmosphere. After that add dropwise oxalyl chloride (309.0 mL, 3.603 mol, 17.86 equiv) using addition funnel for a period of 30 min. Then reaction mixture was heated to 65° C. for 72 hrs. After completion of reaction (TLC—5% MeOH:DCM; TLC charring—Phosphomolybdic acid) solvent was evaporated on rota evaporator to get crude compound. The crude compound was washed with diethyl ether (2×500 mL), cold ethyl acetate (2×400 mL), and 30% ethyl acetate:hexane (1000 mL). After washing solvent was decanted and dried on high vacuum to get 50.0 g of crude WLS-44c as a brown gummy liquid. ¹H NMR (500 MHz, CDCl₃): δ in ppm=4.32 (s, 4H), 3.65 (t, 4H, J=7.4 Hz), 1.65-1.72 (m, 4H), 1.38 (dt, 4H, J=22.5 Hz, 7.4 Hz), 0.97 (t, 6H, J=7.4 Hz). MS: m/z calcd for C₁₁H₂₂Cl₂N₂ ([M−Cl]⁺), 217.76; found 217.07.

Preparation of compound WLS-44d: WLS-44c (50.0 g, 0.197 mol, 1.0) was taken in clean and dry 1 Lit single neck RBF under argon atmosphere. Add 400 mL of DCM to RBF containing SM under argon atmosphere. Then added aq solution of KPF6 (36.35 g, 0.197 mol, 1.0 equiv, in 200 mL of water). Stir the reaction mixture at rt for 1 h. After completion of reaction (TLC—10% MeOH:DCM; TLC charring—Phosphomolybdic acid), the reaction mixture was poured into ice water (400 mL), and extracted with DCM (2×500 mL). The combined organic layer washed with water (400 mL) and dried over sodium sulphate, filtered and evaporated to dryness. Then, residue was dissolved in DCM (15 mL) and product was precipitate by dropwise addition of diethyl ether (600 mL) under stirring. The solvent was decant and solid was dried under high vacuum. The above precipitation procedure repeat one more time to get 54.0 g (75% yield) of WLS-44d as a white solid. ¹H NMR (500 MHz, CDCl₃): δ in ppm=4.10 (s, 4H), 3.54 (t, 4H, J=7.6 Hz), 1.62-1.68 (m, 4H), 1.36 (td, 4H, J=15.0 Hz, 7.3 Hz), 0.96 (t, 6H, J=7.2 Hz).

Preparation of compound WLS-44: WLS-44d (50.0 g, 0.138 mol, 1.0 equiv) was taken in clean and dry 1 Lit single neck RBF under argon atmosphere. Add 250 mL of Dry MeCN to RBF containing SM under argon atmosphere. Then, added sodium azide (13.44 g, 0.207 mol, 1.5 equiv) portion wise for the period of 10 min. Stir the reaction mixture at rt for 2.5 h. After completion of reaction (TLC—5% MeOH:DCM; TLC charring - ninhydrin), reaction mixture was filtered through a pad of celite and washed with MeCN (50 mL). The organic layer was evaporated to dryness. The crude compound was cool to −20° C. using dry ice and methanol bath, then hexane was added, after some timethe compound forms solid then hexane was decanted and solid was dried on high vacuum to get 39.0 g (77% yield) of WLS-44 as a light yellow solid. ¹H NMR (400 MHz, CDCl₃): δ in ppm=3.91 (s, 4H), 3.43 (t, 4H, J=7.7 Hz), 1.60-1.67 (m, 4H), 1.36 (dt, 4H, J=22.4 Hz, 7.4 Hz), 0.95 (t, 6H, J=7.4 Hz). ¹⁹F NMR (400 MHz, CDCl₃): δ in ppm=−73.10 and −74.99. MS: m/z calcd for C₁₁H₂₂F₆N₅P ([M−PF₆]⁺), 224.33; found 224.20. IR (KBr pellet): N₃ (2173 cm⁻¹)

### Synthesis of 2-azido-1-hexyl-3-methyl-4,5-dihydro-1H-imidazol-3-ium hexafluoro-phosphate(V) (WLS-45)

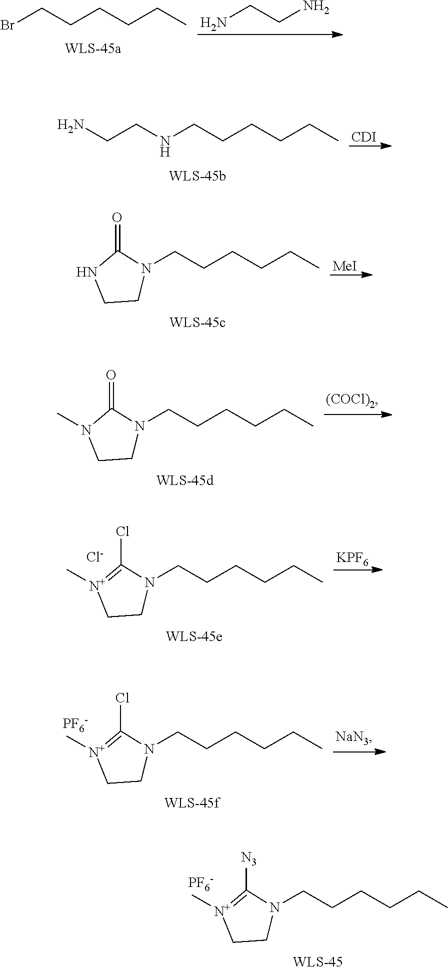

Preparation of compound WLS-45b: In a clean and dry three-neck 3 Lit round bottom flask, ethane-1,2-diamine (1133 mL, 16.972 mol, 28.0 equiv) was placed with a magnetic stirring bar, and compound WLS-45a (100 g, 0.606 mol, 1.0 equiv) was added dropwise at 0° C. by using addition funnel. After finishing the addition, the reaction mixture was warmed to 25° C., and left undisturbed for an additional 1 h. Then, 600 mL of hexane was added into the reaction mixture and stirred vigorously for 16 h at 25° C. TLC showed the reaction was completed, staring material was consumed and the new spot was formed (TLC—10% MeOH:EtOAc; TLC charring—Phosphomolybdic acid). The hexane layer was separated by using separatory funnel, dried over sodium sulfate, and evaporated to dryness under reduced pressure to get compound WLS-45b (60.0 g) as a crude colorless oil. Crude compound was directly used for next step without any further purification. ¹H NMR (400 MHz, CDCl₃): δ in ppm=2.82-2.79 (m, 2H), 2.66 (t, 2H, J=5.9 Hz), 2.60 (t, 2H, J=7.2 Hz), 1.52-1.45 (m, 2H), 1.36-1.27 (m, 9H), 0.89 (t, 3H, J=6.9 Hz). MS: m/z calcd for C₈H₂₀N₂ ([M+H]⁺), 145.26; found 145.00.

Preparation of compound WLS-45c: WLS-45b (60.0 g, 0.416 mol, 1.0 equiv) was taken in clean and dry 1 Lit single neck RBF under argon atmosphere. Then add 600 mL of THF to RBF. Cool the RB in ice bath (0° C. ). Add portion wise 1,1′-Carbonyldiimidazole (69.46 g, 0.428 mol, 1.03 equiv) to RM for period of 10 min. The reaction mixture was stir at 15° C. for 16 h. TLC showed the reaction was completed, staring material was consumed and the product was formed (TLC—10% MeOH:EtOAc; TLC charring—Phosphomolybdic acid). After completion of reaction, reaction mixture was filtered, filter cake was washed with THF (100 mL). Filtrate was dried and purified on silica gel column chromatography (100-200 mesh). The product was eluted with 80% ethyl acetate: hexane to EtOAc. Fraction containing product was evaporated to get 58.0 g (82% yield) of WLS-45c as a colorless oil. ¹H NMR (500 MHz, CDCl₃): δ in ppm=4.84 (s, 1H), 3.41 (s, 4H), 3.17 (t, 2H, J=7.6 Hz), 1.49 (q, 2H, J=7.1 Hz), 1.30 (d, 6H, J=15.0 Hz, 2.1 Hz), 0.88 (t, 3H, J=7.6 Hz). MS: m/z calcd for C₉H₁₈N₂O ([M+H]⁺), 171.26; found 171.10.

Preparation of compound WLS-45d: WLS-45c (48.0 g, 0.282 mol, 1.0 equiv) was taken in clean and dry 2 Lit three neck RBF under argon atmosphere. Then, add 800 mL of dry DMF to RBF containing SM. Cool the RB in ice bath (Temp. ° C.). Then, add portion wise 60% NaH (8.13 g, 0.338 mol, 1.2 equiv) to RM for period of 20 min. at 0° C. and stir 45 min at same temp. Then add dropwise methyl iodide (53 mL, 0.851 mol, 3.02 equiv) to the reaction mixture at 0° C. for duration of 30 min. Then allow the RM to rt and stir for 3 h. TLC showed the reaction was completed, staring material was consumed and the new spot was formed (TLC—EtOAc; TLC charring - Phosphomolybdic acid). After completion of reaction, reaction mixture was cool to 0° C. in ice bath and quenched with ice cold water (200 mL). Then extracted with ethyl acetate 3×300 mL). The organic layer was washed with ice cold water (2×500 mL) and dried over sodium sulfate, filtered and concentrated to dryness. The crude product was purified by silica gel column chromatography (100-200 mesh). The product was eluted with 40%-50% ethyl acetate:hexane. The fraction containing product was evaporated to get 36.4 g (70% yield) of WLS-45d as a white colour solid. ¹H NMR (400 MHz, CDCl₃): δ in ppm=3.27 (s, 4H), 3.17 (t, 2H, J=7.6 Hz), 2.78 (s, 3H), 1.50-1.45 (m, 2H), 1.29 (s, 7H), 0.88 (t, 3H, J=6.9 Hz).

Preparation of compound WLS-45e: WLS-45d (43.0 g, 0.233 mol, 1.0 equiv) was taken in clean and dry 2 Lit three neck RBF under argon atmosphere. Then add 430 mL of dry toluene to RBF containing SM under argon atmosphere. After that add dropwise oxalyl chloride (300 mL, 3.498 mol, 15 equiv) using addition funnel for a period of 30 min at rt. Then, reaction mixture was heated to 65° C. for 72 hrs. After completion of reaction (TLC—5% MeOH:DCM; TLC charring - Phosphomolybdic acid) solvent was evaporated to dryness to get crude compound. The crude compound was precipitate by using DCM−Hexane (three times) and solid was dried under high vacuum to get 48.0 g of crude WLS-45e as an oil. Crude compound was directly used for next step without any further purification. MS: m/z calcd for C₁₀H₂₀Cl₂N₂ ([M−Cl]⁺), 203.73; found 203.43.

Preparation of compound WLS-45f: WLS-45e (48.0 g, 0.201 mol, 1.0 equiv) was taken in clean and dry 21 L single neck RBF and dissolved in 480 mL DCM under argon atmosphere. Then added aq solution of KPF₆ (36.95 g, 0.201 mol, 1.0 equiv in 240 mL of water). Stir the reaction mixture at rt for 2 h. After completion of reaction (TLC—5% MeOH:DCM), the reaction mixture was poured into ice water, and extracted with DCM (2×400 mL). The combined organic layer washed with water (400 mL) and dried over sodium sulphate, filtered and evaporated to dryness. Then, residue was dissolved in DCM and product was precipitate by dropwise addition of hexane under stirring. The solvent was decant and solid was dried under high vacuum. The above precipitation procedure repeat two more times to get 58.35 g (83% yield) of WLS-45f as a yellow solid. ¹H NMR (500 MHz, CDCl₃): δ in ppm=4.14-4.03 (m, 4H), 3.51 (t, 2H, J=7.6 Hz), 3.22 (s, 3H), 1.64 (q, 2H, J=7.1 Hz), 1.31 (d, 6H, J=4.8 Hz), 0.89 (t, 3H, J=6.9 Hz). ¹⁹F NMR (400 MHz, CDCl₃): δ in ppm=−73.16 and −74.68 MS: m/z calcd for C₁₀H₂₀ClF₆N₂P ([M−Cl]⁺), 203.73; found 203.96.

Preparation of compound WLS-45: WLS-45f (58.35 g, 0.167 mol, 1.0 equiv) was taken in clean and dry 1 L single neck RBF and dissolved in 292 mL of Dry MeCN under argon atmosphere. Then, added portion wise sodium azide (16.31 g, 0.251 mol, 1.5 equiv) to the RM and stir at rt for 3 h. After completion of reaction (TLC—5% MeOH:DCM; TLC charring—ninhydrin), reaction mixture was filtered through a pad of celite and washed with MeCN (200 mL). The organic layer was evaporated to dryness. The crude compound was dissolve in minimum amount of MeCN and add by diethylether to form gummy liquid, solvent was decant and compound was dried. Repeat this procedure two times. Then hexane was added to gummy liquid and stir at at -30° C. to get solid. Solvent was decanted and solid was dried to get 55.0 g (93% yield) of WLS-45 as a light yellow solid. ¹H NMR (400 MHz, CDCl₃): δ in ppm=3.97-3.92 (m, 2H), 3.88-3.83 (m, 2H), 3.37 (t, 2H, J=7.7 Hz), 3.19 (s, 3H), 1.63-1.57 (m, 2H), 1.31 (s, 6H), 0.89 (t, 3H, J=6.7 Hz). ¹⁹F NMR (400 MHz, CDCl₃): δ in ppm=−73.45 and −74.97. MS: m/z calcd for C₁₀H₂₀F₆N₅P ([M−PF₆]⁺), 210.30; found 210.19. IR (KBr pellet): N3 (2173 cm⁻¹)

### Synthesis of 2-azido-1,3-dihexyl-4,5-dihydro-1H-imidazol-3-ium hexafluorophosphate(V) (WLS-46)

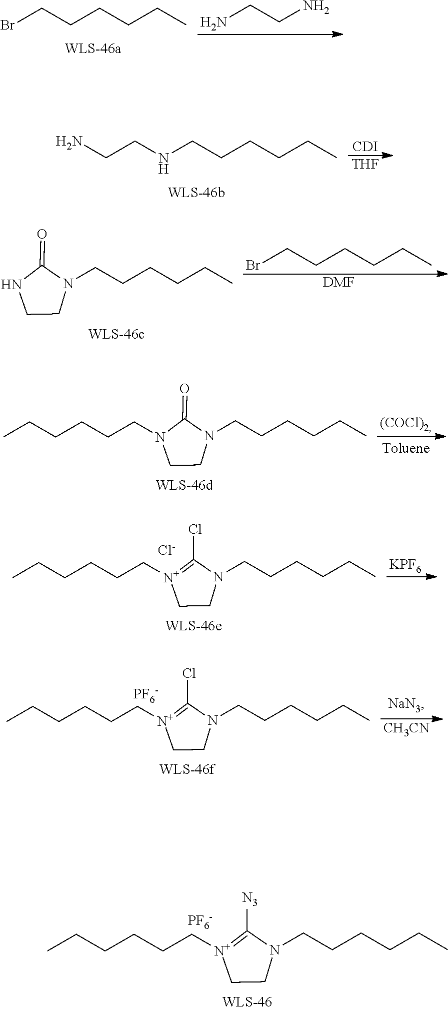

Preparation of compound WLS-46b: In a clean and dry three-neck 2 Lit round bottom flask, ethane-1,2-diamine (1133 mL, 16.972 mol, 28.0 equiv) was placed with a magnetic stirring bar, and compound WLS-46a (100 g, 0.606 mol, 1.0 equiv) was added dropwise at 0° C. by using addition funnel. After finishing the addition, the reaction mixture was warmed to 25° C., and left undisturbed for an additional 1 h. Then, 600 mL of hexane was added into the reaction mixture and stirred vigorously for 16 h at 25° C. TLC showed the reaction was completed, staring material was consumed and the new spot was formed (TLC—10% MeOH:EtOAc; TLC charring—Phosphomolybdic acid). The hexane layer was separated by using separatory funnel. Again 300 mL of hexane was added to amine layer and stir for 4 h. After that hexane layer was separated and combined with previous hexane layer, dried over sodium sulphate, and evaporated to dryness under reduced pressure to get compound WLS-46b (60 g) as a crude colorless liquid. MS: m/z calcd for C8H20N2 ([M+H]⁺), 145.26; found 145.00.

Preparation of compound WLS-46c: WLS-46b (40.0 g, 0.277 mol, 1.0 equiv) was taken in clean and dry 1 Lit single neck RBF and dissolved by adding 400 mL of THF. Cool the RB in ice bath (Temp. 0° C. ). Add portion wise 1,1′-Carbonyldiimidazole (45.13 g, 0.278 mol, 1.0 equiv) to RM for period of 15 min. The reaction mixture was stir at 15° C. for 16 h. TLC showed the reaction was completed, staring material was consumed and the new spot was formed (TLC—10% MeOH:EtOAc; TLC charring—Phosphomolybdic acid). After completion of reaction, reaction mixture was filter through celite pad and washed with ethyl acetate (150 mL). The combined filtrate was evaporated to dryness and purified by using silica gel column chromatography (100-200 mesh). The product was eluted with 30% ethyl acetate: hexane to ethyl acetate. Fraction containing product was evaporated to get 29.0 g (61% yield) of WLS-46c as a white solid. ¹H NMR (500 MHz, CDCl₃): δ in ppm=4.84 (s, 1H), 3.41 (s, 4H), 3.17 (t, 2H, J=7.6 Hz), 1.49 (q, 2H, J=7.1 Hz), 1.30 (d, 6H, J=2.1 Hz), 0.87-0.90 (m, 3H)MS: m/z calcd for C₉H₁₈N₂O ([M+H]⁺), 171.26; found 171.10.

Preparation of compound WLS-46d: WLS-46c (29.0 g, 0.170 mol, 1.0 equiv) was taken in clean and dry 1 Lit two neck RBF and dissolved by adding 464 mL of DMF under argon atmosphere. Cool the RB in ice bath (Temp. 0° C. ). Then, add portion wise NaH (8.18 g, 0.204 mol, 1.2 equiv) to the RM for period of 20 min. at 0° C. Then add dropwise bromo hexane (71.56 mL, 0.512 mol, 3.0 equiv) to the reaction mixture at 0° C. for duration of 30 min. Then, allow the RM to rt and stir for 3 h. TLC showed the reaction was completed, staring material was consumed and the new spot was formed (TLC—50% EtOAc:Hexane; TLC charring—Phosphomolybdic acid). After completion of reaction, reaction mixture was cool to 0° C. in ice bath and quenched with ice cold water. Then extracted with ethyl acetate (2×700 mL). The combined organic layer washed with ice cold water (2×1000 mL), dried over sodium sulfate, filtered and concentrated to dryness. The crude product was purified by silica gel column chromatography (100-200 mesh). The product was eluted with 10%45% ethyl acetate:hexane. The fraction containing product was evaporated to get 29.0 g (67% yield) of WLS-46d as a yellow liquid. ¹H NMR (500 MHz, CDCl₃): δ in ppm=3.27 (s, 4H), 3.16 (t, 4H, J=7.6 Hz), 1.48 (q, 4H, J=7.1 Hz), 1.29 (s, 12H), 0.88 (t, 6H, J=6.9 Hz). MS: m/z calcd for C₁₅H₃₀N₂₀ ([M+H]⁺), 255.42; found 255.27.

Preparation of compound WLS-46e: WLS-46d (29.0 g, 0.114 mol, 1.0 equiv) was taken in clean and dry 1 Lit two neck RBF and dissolved by adding 240 mL of dry toluene under argon atmosphere. Then add dropwise oxalyl chloride (146.5 mL, 1.708 mol, 15.0 equiv) to reaction mixture using addition funnel for a period of 30 min. Then reaction mixture was heated to 70° C. for 64 hrs. After completion of reaction (TLC—5% MeOH:DCM; TLC charring - Phosphomolybdic acid) solvent was evaporated to dryness to get crude compound. The crude compound was dissolve in minimum amount of ethyl acetate and precipitate by adding dropwise hexane. Solvent was decant and solid was dried. The above precipitation procedure repeat one more time to get 34.0 g (96% yield) of WLS-46e as brown colour semi solid. ¹H NMR (500 MHz, CDCl₃): δ in ppm=4.33 (s, 4H), 3.65 (s, 4H), 1.69 (s, 4H), 1.30 (d, 12H, J=28.2 Hz), 0.90 (t, 6H, J=6.2 Hz).

Preparation of compound WLS-46f: WLS-46e (34.0 g, 0.110 mol, 1.0 equiv) was taken in clean and dry 1 Lit single neck RBF and dissolved by adding 196 mL of DCM under argon atmosphere. Then added aq. solution of KPF₆ (20.20 g, 0.110 mol, 1.0 equiv, in 110 mL of water). Stir the reaction mixture at rt for 1 h. After completion of reaction (TLC—10% MeOH:DCM; TLC charring—Phosphomolybdic acid), the reaction mixture was poured into ice water, and extracted with DCM (2×400 mL). The combined organic layer washed with water (400 mL) and dried over sodium sulphate, filtered and evaporated to dryness. Then, residue was dissolved in DCM (50 mL) and product was precipitate by dropwise addition of diethyl ether (500 mL) under stirring. The solvent was decanted and solid was dried under high vacuum. The above precipitation procedure repeat one more time to get 37.0 g (80% yield) of WLS-46f as a light brown solid. ¹H NMR (400 MHz, CDCl₃): δ in ppm=4.10 (s, 4H), 3.54 (t, 4H, J=7.6 Hz), 1.65 (q, 4H, J=7.3 Hz), 1.32 (d, 12H, J=2.1 Hz), 0.88-0.91 (m, 6H). ¹⁹FNMR (400 MHz, CDCl₃): 6 in ppm=-72.87 and -74.76. MS: m/z calcd for C₁₅H₃₀ClF₆N₂P ([M−PF₆]⁺), 273.86; found 273.25.

Preparation of compound WLS-46: WLS-46f (37.0 g, 0.088 mol, 1.0 equiv) was taken in clean and dry 1 Lit single neck RBF and dissolved by adding 185 mL of Dry MeCN under argon atmosphere. Then, added sodium azide (8.61 g, 0.132 mol, 1.5 equiv) to the RM and stir at rt for 2.5 h. After completion of reaction (TLC—5% MeOH:DCM; TLC charring—ninhydrin), reaction mixture was filtered through a pad of celite and washed with MeCN (50 mL). The organic layer was evaporated to dryness. The crude compound was dissolve in DCM (15 mL) and precipitate by adding dropwise hexane (500 mL). Solvent was decanted and solid was dried under high vacuum to get 27.0 g (72% yield) of WLS-46 as a light yellow solid. ¹H NMR (400 MHz, CDCl₃): δ in ppm=3.92 (s, 4H), 3.45 (t, 4H, J=7.7 Hz), 1.64 (q, 4H, J=7.4 Hz), 1.31 (s, 12H), 0.88-0.91 (m, 6H). ¹⁹F NMR (400 MHz, CDCl₃): δ in ppm=−73.13 and −75.02. MS: m/z calcd for C₁₅H₃₀F₆N₅P ([M−PF₆]⁺), 280.44; found 280.26. IR (KBr pellet): N3 (2167 cm⁻¹).

### Synthesis of 2-azido-1,3-diethyl-4,5-dihydro-1H-imidazol-3-ium hexafluorophosphate(V) (WLS-56)

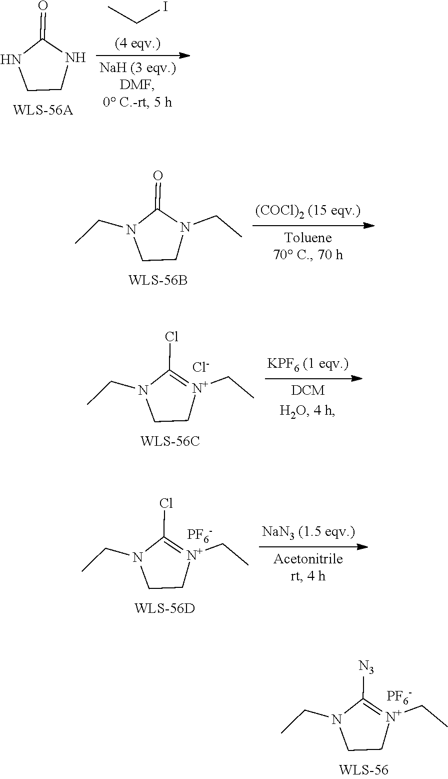

1,3-diethylimidazolidin-2-one (WLS-56B): To a stirred solution of imidazolidin-2-one (WLS-56A) (20 g, 0.2325 mol, 1.0 equiv) in DMF (300 mL) at 0° C. was added sodium hydride (60% dispersion in oil) (28 g, 0.696 mol, 3.0 equiv) portion wise over a period of 1 h, and further stirred for another 1 h. After that ethyl iodide (73.9 mL, 0.9808 mol, 4.0 equiv) was added dropwise over a period of 50 mins at 0° C. Then the reaction mixture was allowed to rt and stirred for 5 h. Progress of the reaction was monitored by TLC. Above reaction was diluted with ice water (300 mL) and extracted with ethyl acetate (2×500 mL). Combined organic layers was washed with cold brine solution (3×100 mL), dried over Na₂SO₄ and concentrated under reduced pressure. The crude was purified by column chromatography over silica gel (230-400 mesh) eluted in 30% EA/Hexane to get a light-yellow oil (21 g, 63%). 1H NMR (500 MHz, CDCl₃): δ in ppm=3.28 (s, 4H), 3.24 (q, 4H, J=7.3 Hz), 1.10 (t, 6H, J=7.2 Hz). MS (ESI) 143.15 (M+1)⁺.

2-chloro-1,3-diethyl-4,5-dihydro-1H-imidazol-3-ium chloride (WLS-56C): To a solution of 1,3-diethylimidazolidin-2-one (WLS-56B) (36 g, 0.2531 mol) in toluene (360 mL) was added oxalyl chloride (325 mL, 3.796 mol, 15 equiv) dropwise over a period of 1 hat 0° C. under argon. Then the mixture was stirred at 70° C. for 70 h. Progress of the reaction was monitored by TLC. The reaction was concentrated under reduced pressure to afford a crude mass which was treated with diethyl ether (2×200 mL). The solid was precipitated, filter off, washed with diethyl ether (3×30 mL) and dried under vacuum to afford (40 g, crude), which was used for the next step without further purification. 1H NMR (500 MHz, CDCl₃): 6 in ppm=4.36 (s, 4H), 3.73 (q, 4H, J=7.3 Hz), 1.35 (t, 6H, J=7.2 Hz). MS (ESI) 161.14 (M−Cl)⁺.

2-chloro-1,3-diethyl-4,5-dihydro-1H-imidazol-3-ium hexafluorophosphate(V) (WLS-56D) : To a stirred solution of 2-chloro-1,3-diethyl-4,5-dihydro-1H-imidazol-3-ium chloride (WLS-56C) (40 g, 0.2040 mol, 1.0 equiv) in DCM (400 mL) was added a solution of KPF₆ (37.54 g, 0.2040 mol, 1.0 equiv) in water (200 mL) dropwise over a period of 50 mins at rt. Above reaction mixture was stirred at rt for 4 h. Progress of the reaction was monitored by TLC. Then the mixture was filtered through a celite bed, washed with DCM (3×80 mL). The organic layer was washed with water (3×100 mL) dried over Na₂SO₄, filtered and evaporated to dryness. The gummy residue was re-dissolved in DCM (50 mL) and added dropwise to precool diethyl ether (150 mL) at −78 oC under stirring. A brownish solid was precipitate out. The solid was filtered, washed with ether (2×50 mL) and dried under vacuum to get the desired compound 2-chloro-1,3-diethyl-4,5-dihydro-1H-imidazol-3-ium hexafluorophosphate(V) (WLS-56D) (38 g, 60%). 1H NMR (500 MHz, CDCl₃): δ in ppm=4.12 (s, 4H), 3.65 (m, 4H), 1.33 (m, 6H). MS (ESI) 161.14 (M-PF6)⁺.

2-azido-1,3-diethyl-4,5-dihydro-1H-imidazol-3-ium hexafluorophosphate(V) (WLS-56D): To a precool solution of 2-chloro-1,3-diethyl-4,5-dihydro-1H-imidazol-3-ium hexafluoro-phosphate(V) (WLS-56D) (WLS-56D) (36 g, 0.1176 mol, 1.0 equiv) in acetonitrile (360 mL) was added a sodium azide (11.40 g, 0.1765 mol, 1.0 equiv) portion wise over a period of 20 mins under N₂ atmosphere. Above reaction mixture was stirred at rt for 5 h. Progress of the reaction was monitored by TLC. Then the mixture was filtered through a celite bed, washed with acetonitrile (2×100 mL). The filtrate was evaporated under vacuum to afford a gummy mass. The residue was again dissolved in DCM (45 mL) and dropwise added to diethyl ether (200 mL) under stirring, at −78° C. The solid was precipitated, filtered and washed with ether (2×50 mL), and dried under vacuum to get the desired compound (30 g, 81%). 1H NMR (400 MHz, CDCl₃): δ in ppm=3.93 (s, 4H), 3.54 (q, 4H, J=7.3 Hz), 1.31 (t, 6H, J=7.4 Hz). MS (ESI) 168.23 (M+)⁺. 19F NMR (400 MHz, CDCl₃): δ in ppm=−73.13 and −75.03. IR (KBr pellet): N₃ (2175.31 cm⁻¹).

### Synthesis of 2-azido-1,3-dipropyl-4,5-dihydro-1H-imidazol-3-ium hexafluorophosphate(V) (WLS-57)

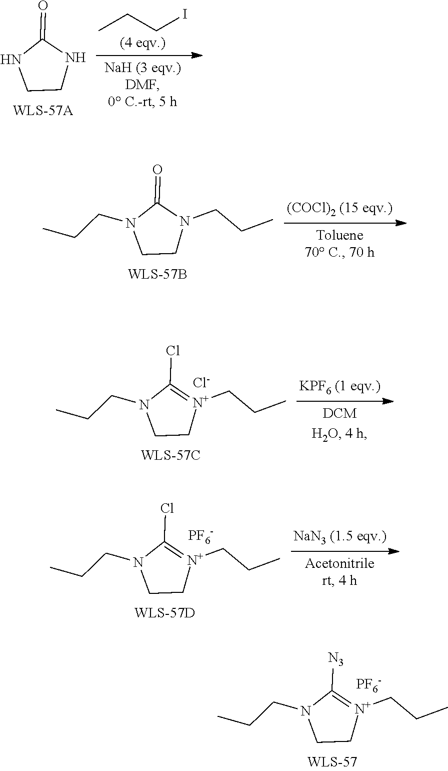

1,3-dipropylimidazolidin-2-one (WLS-57B): To a stirred solution of imidazolidin-2-one (15 g, 0.17 mol, 1.0 equiv) in DMF (225 mL) was added sodium hydride (20.9 g, 0.52 mol.) portion wise at 0° C. over a period of 40 min, and kept for 1 h. Then 1-bromopropane (63.5 mL, 0.69 mol, 1.2 equiv) was added dropwise over a period of 30 min. and stirred for 5 h at rt. Progress of the reaction was monitored by TLC. Above reaction was diluted with ice water (300 mL) and extracted with ethyl acetate (3×400 mL). Combined organic layer was washed with cold brine solution (3×100 mL), dried over Na₂SO₄ and concentrated under vacuum. The crude was purified by column chromatography over silica gel (230-400 mesh) eluted in 30% EA/Hexane to yield1,3-dipropylimidazolidin-2-one (WLS-57B) as a light yellow oil (21 g, 71%). 1H NMR (500 MHz, CDCl₃): δ in ppm=3.21 (s, 4H), 3.07 (t, 4H, J=7.6 Hz), 1.45 (td, 4H, J=14.8 Hz, 7.6 Hz), 0.83 (t, 6H, J=7.2 Hz). MS (ESI) 171.25 (M+1)⁺.

2-chloro-1,3-dipropyl-4,5-dihydro-1H-imidazol-3-ium chloride (WLS-57C): To a cool solution of 1,3-dipropylimidazolidin-2-one (WLS-57B) (15 g, 0.088 mol, 1.0 equiv) in toluene (150 mL) was added oxalyl chloride (113 mL, 1.32 mol., 15.0 equiv) dropwise over a period of 30 min under argon atmosphere. Above mixture was stirred at 70 oC for 72 h. Progress of the reaction was monitored by TLC. Then the reaction was concentrated under reduced pressure to afford a crude mass which was treated with n-hexane (3×75 mL) followed by diethyl ether ((2×100 mL) to get a brownish solid. The solid was dried under vacuum to afford (18 g, crude) which was used for the next step without further purification. 1H NMR (500 MHz, CDCl₃): δ in ppm=4.32 (s, 4H), 3.61 (t, 4H, J=7.6 Hz), 1.76 (td, 4H, J=14.8 Hz, 7.6 Hz), 0.99 (t, 6H, J=7.6 Hz). MS (ESI) 189.18 (M−C1)⁺.

2-chloro-1,3-dipropyl-4,5-dihydro-1H-imidazol-3-ium hexafluorophosphate(V) (WLS-57C): To a stirred solution of 2-chloro-1,3-dipropyl-4,5-dihydro-1H-imidazol-3-ium chloride (WLS-57C) (16 g, 0.0714 mol, 1.0 equiv) in DCM (160 mL) was added a solution of KPH₆ (13.14 g, 0.0714 mol., 1.0 equiv) in 80 mL of water over a period of 30 mins at rt. Above reaction mixture was stirred for 3 h. Progress of the reaction was monitored by TLC. Then the mixture was filtered through a celite bed, the bed was washed with DCM (2×100 mL). The combined organic layer was washed with water (3×100 mL), dried over Na₂SO₄ and evaporated to dryness. The residue was again dissolved in DCM (30 mL) and then added diethyl ether (200 mL) under stirring. The solid was precipitate out which was filtered and washed with ether (2×50 mL), dried under vacuum to afford 2-chloro-1,3-dipropyl-4,5-dihydro-1H-imidazol-3-ium hexafluorophosphate(V) (WLS-57C) as a reddish solid (16 g, 67%). 1H NMR (500 MHz, CDCl₃): 6 in ppm=4.11 (s, 4H), 3.52 (t, 4H, J=7.6 Hz), 1.71 (td, 4H, J=15.1 Hz, 7.6 Hz), 0.97 (t, 6H, J=7.2 Hz). MS (ESI) 189.19 (M−PF₆)⁺.

2-azido-1,3-dipropyl-4,5-dihydro-1H-imidazol-3-ium hexafluorophosphate(V) (WLS-57) : To a stirred cool solution of 2-chloro-1,3-dipropyl-4,5-dihydro-1H-imidazol-3-ium hexafluorophosphate(V) (WLS-57D) (11 g, 0.032 mol, 1.0 equiv) in acetonitrile (110 mL) was added sodium azide (3.2 g, 0.049 mol., 1.5 equiv) portion wise over a period of 20 mins under nitrogen. Above reaction mixture was stirred for 3 h. Progress of the reaction was monitored by TLC. Then the mixture was filtered through a celite bed; washed with acetonitrile (2×100 mL). The filtrate was evaporated under vacuum to afford a crude mass. The residue was dissolved in DCM (30 mL) and then added diethyl ether (200 mL) under stirring. The solid was thrown out which was filtered and washed with ether (2×50 mL), dried under vacuum to get 2-azido-1,3-dipropyl-4,5-dihydro-1H-imidazol-3-ium hexafluorophosphate(V) (WLS-57) as a brownish solid (10 g, 89%). 1H NMR (400 MHz, CDCl₃): δ in ppm=3.93 (s, 4H), 3.42 (t, 4H, J=7.6 Hz), 1.70 (td, 4H, J=15 Hz, 7.6 Hz), 0.97 (t, 6H, J=7.4 Hz). MS (ESI) 196.25 (M—PF6)⁺. 19F NMR (400 MHz, CDCl₃): δ in ppm=−73.3 and −74.8. IR (KBr pellet): N₃ (2175 cm⁻¹).

### Synthesis of 2-azido-1,3-diisopropyl-4,5-dihydro-1H-imidazol-3-ium hexafluoro-phosphate(V) (WLS-58)

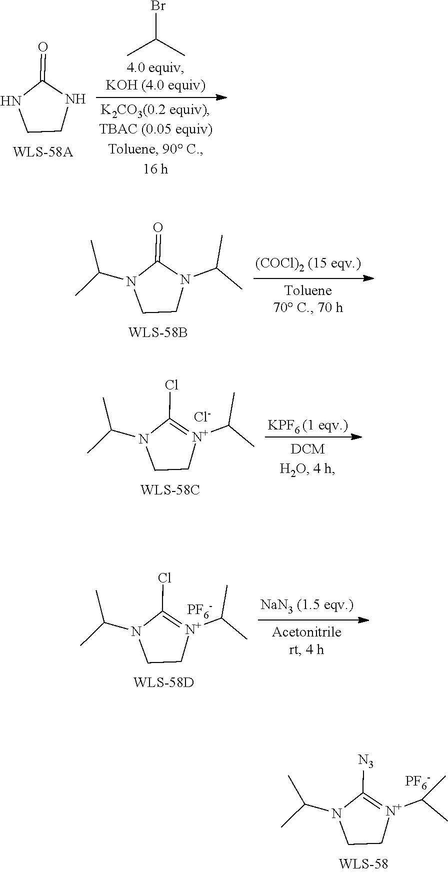

1,3-diisopropylimidazolidin-2-one (WLS-58B): To a stirred solution of imidazolidin-2-one (WLS-58B) (20 g, 0.23 mol, 1.0 equiv) in toluene (340 mL) was added potassium hydroxide (52 g, 0.92 mol., 4.0 equiv), Potassium carbonate (6.41 g, 0.046 mol., 0.2 equiv) and tetrabutylammonium chloride (3.22 g, 0.011 mol., 0.05 equiv) at rt under N₂ atmosphere. Then 2-bromo propane (87.24 mL, 0.92 mol., 4.0 equiv) was added slowly. Above reaction mixture was stirred at 90° C. for 20 h. Progress of the reaction was monitored by TLC. Then the mixture was diluted with ice water (200 mL) and extracted with DCM (2×400 mL). Combined organic phase was washed with brine solution (2×100 mL) dried over Na₂SO₄ and concentrated under reduced pressure. The crude was purified by column chromatography over silica gel (230-400 mesh) eluted in 30% EA/Hexane to get 1,3-diisopropylimidazolidin-2-one (WLS-58B) as a pale yellow syrup (18 g, 45%). 1H NMR (400 MHz, CDCl₃): δ in ppm=4.09 (m, 2H), 3.17 (s, 4H), 1.06 (d, 12H, J=6.7 Hz) MS (ESI) 171.24 (M+1)⁺.

2-chloro-1,3-diisopropyl-4,5-dihydro-1H-imidazol-3-ium chloride (WLS-58C): To a ice cool solution of 1,3-diisopropylimidazolidin-2-one (WLS-58B) (15 g, 0.0588 mol, 1.0 equiv) in toluene (100 mL) was added oxalyl chloride (76.2 mL, 0.088 mol., 15.0 equiv) dropwise over a period of 30 min under argon atmosphere. Above mixture was stirred at 70° C. for 72 h. Progress of the reaction was monitored by TLC. After that the reaction mixture was concentrated under reduced pressure to afford a crude mass which was treated with 40% EA/Hexane (3×75 mL) and stirred for 30 min. Then the solid was precipitated out, filtered and washed with diethyl ether (2×50 mL). The compound was dried under vacuum to afford give 2-chloro-1,3-diisopropyl-4,5-dihydro-1H-imidazol-3-ium chloride (WLS-58C) as a brownish solid (13 g, crude) which was used in the next step without further purification. 1H NMR (500 MHz, CDCl₃): δ in ppm=4.31 (m, 6H), 1.41 (d, 12H, J=6.5 Hz) MS (ESI) 189.14 (M-C1)⁺.

2-chloro-1,3-diisopropyl-4,5-dihydro-1H-imidazol-3-ium hexafluorophosphate(V) (WLS-58D): To a stirred solution of 2-chloro-1,3-diisopropyl-4,5-dihydro-1H-imidazol-3-ium chloride (WLS-58C) (20 g, 0.0888 mol, 1.0 equiv) in DCM (200 mL) was added a solution of KPH₆ (16.3 g, 0.0888 mol., 1.0 equiv) in water (100 mL) dropwise over a period of 30 min. Above reaction mixture was stirred for 4 h. Progress of the reaction was monitored by TLC. Then the mixture was filtered through a celite bed, the bed was washed with DCM (2×130 mL). The Organic layer was washed with water (3×100 mL), dried over Na₂SO₄, filtered and evaporated to dryness. The residue was dissolved in DCM (25 mL) and then added diethyl ether (165 mL) under stirring. The solid precipitated was filtered and washed with ether (2×50 mL), dried under vacuum to afford 2-chloro-1,3-diisopropyl-4,5-dihydro-1H-imidazol-3-ium hexafluorophosphate(V) (WLS-58D) as a light brown solid (18 g, 61%). 1H NMR (500 MHz, CDCl₃): δ in ppm=4.29 (m, 2H), 4.07 (s, 4H), 1.37 (d, 12H, J=6.9 Hz), MS (ESI) 189.15 (M−PF₆)⁺.

2-azido-1,3-diisopropyl-4,5-dihydro-1H-imidazol-3-ium hexafluorophosphate(V) (WL S-58): To a cool stirred solution of 2-chloro-1,3-diisopropyl-4,5-dihydro-1H-imidazol-3-ium hexafluorophosphate(V) (WLS-58D) (18 g, 0.032 mol, 1.0 eqiv) in acetonitrile (180 mL) was added sodium azide (5.25 g, 0.080 mol., 1.5 eqiv) portion wise over a period of 20 mins under N₂ atmosphere. Above reaction mixture was stirred for 4 h. Progress of the reaction was monitored by TLC. Then the mixture was filtered through a celite bed, the bed was washed with acetonitrile (2×100 mL). The filtrate was evaporated under vacuum to afford a crude. The residue was dissolved in DCM (25 mL) and then added diethyl ether (150 mL) under stirring. The solid precipitated was filtered, washed with ether (2×50 mL) and dried under vacuum to afford 2-azido-1,3-diisopropyl-4,5-dihydro-1H-imidazol-3-ium hexafluorophosphate(V) (WLS-58) as a brownish solid (17 g, 92%). 1H NMR (500 MHz, CDCl₃): δ in ppm=4.18 (m, 2H), 3.86 (s, 4H), 1.33 (d, 12H, J=6.2 Hz) MS (ESI) 196.26 (M—PF₆)⁺. 19F NMR (500 MHz, CDCl₃): δ in ppm=−72.86 and −74.37. IR (KBr pellet): N₃ (2165 cm⁻¹).

### Synthesis of 2-azido-1,3-di ((E)-pent-2-en-1-yl)-4,5-dihydro-1H-imidazol-3-ium hexafluoro-phosphate(V) (WLS-60)

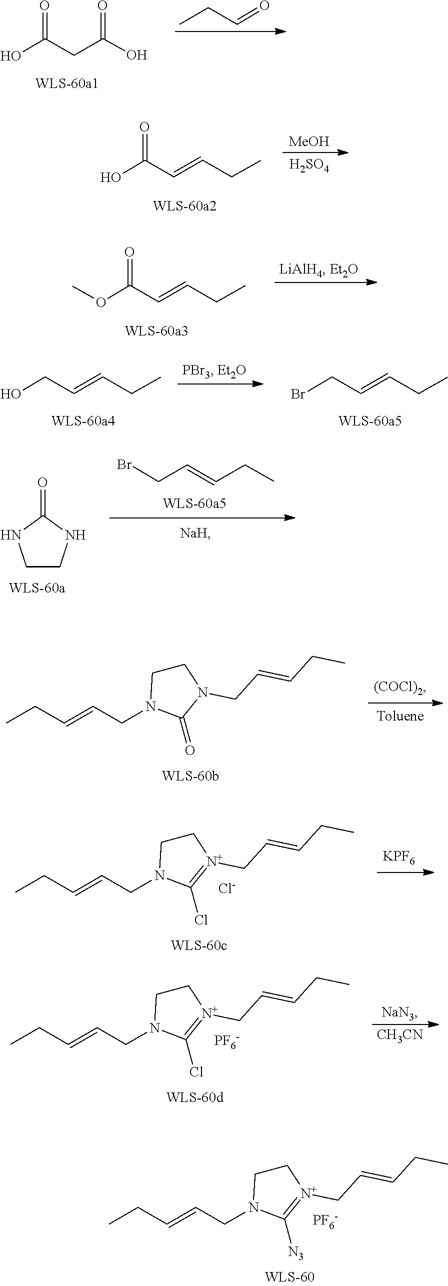

Preparation of compound WLS-60a2: WLS-60a1 (41.60 g, 0.400 mol, 1.0 equiv) was taken in clean and dry 500 mL 2 neck RBF under argon atmosphere. Then, added 41 mL of Pyridine to RBF containing SM. Add dropwise propionaldehyde (30.23 mL, 0.519 mol, 1.3 equiv) to reaction mixture using addition funnel. Then, reaction mixture was heated reflux 70° C. for 4 h. After completion of reaction (TLC—10% MeOH:DCM), cool the reaction mixture with rt. Added 50% H₂SO₄ up to pH <2. Water was added and extract with EtOAc (2×500 mL). The combined the organic layer dried on sodium sulphate, filtered and evaporated to dryness to get 32.0 g (80% yield) WLS-60a2 as a colourless oil. The WLS-60a2 was directly used for next reaction without any further purification. ¹H NMR (500 MHz, CDCl₃): δ in ppm=10.63 (bs, 1H), 7.15 (dt, 1H, J=15.4 Hz, 6.4 Hz), 5.83 (dt, 1H, J=15.8 Hz, 1.7 Hz), 2.29-2.24 (m, 2H), 1.09 (t, 3H, J=7.2 Hz).

Preparation of compound WLS-60a3: WLS-60a2 (32.0 g, 0.320 mol, 1.0 equiv) was taken in clean and dry 500 mL single neck RBF under argon atmosphere was added EtOH (73 mL) followed by toluene (30 mL). Cool the RB in ice bath and add H2504 (2.75 mL). The reaction mixture was heated at 100° C. for 20 h. After completion of reaction (TLC—10% MeOH:DCM), cool the reaction mixture with rt. The volatiles were evaporated. The residue was extracted with DCM (2×600 mL), washed with sat NaHCO₃ (500 mL) solution followed by water (500 ml). The organic layer dried over sodium sulphate and dried under vacuum to get 31.0 g (76% yield) of WLS-60a3 as a light-yellow oil. The WLS-60a3 was directly used for next reaction without any further purification. ¹H NMR (400 MHz, CDCl₃): δ in ppm=7.08-6.98 (m, 1H), 5.81 (dt, 1H, J=15.7 Hz, 1.7 Hz), 4.21-4.15 (m, 2H), 2.26-2.15 (m, 2H), 1.28 (t, 3H, J=7.1 Hz), 1.076 (t, 3H, J=7.4 Hz).

Preparation of compound WLS-60a4: Lithium aluminium hydride (12.18 g, 0.321 mol, 1.87 equiv) was taken in clean and dry 2 Lit two neck RBF under argon atmosphere. Then, add 366 mL of dry Diethyl ether, cooled to 0° C. Then AlCl₃ (15.15 g, 0.114 mol, 0.66 equiv, in 611 mL of ether) was drop wise added to RBF per a period of 50 min. After completion of addition allow to rt and stirred for 30 min. Again cooled to 0° C. add dropwise for a period of 20 min WLS-60a3 (22.00 g 0.172 mol, 1.0 equiv). The reaction mixture was allow to rt and stirred for 1 hrs. After completion of reaction (TLC—10% MeOH:DCM, PMA charring) reaction mixture cool to 0° C. Then quenched the reaction mixture with 20% NaOH solution (70 mL), and stir for 45 min. The residue was extracted with ether (2×600 mL), washed with water (500 ml). The organic layer dried over sodium sulphate and dried under vacuum to get 12.0 g (81% yield) of WLS-60a4 as a light yellow oil. The WLS-60a4 was directly used for next reaction without any further purification. ¹H NMR (500 MHz, CDCl₃): δ in ppm=5.77-5.72 (m, 1H), 5.66-5.60 (m, 1H), 4.09 (t, 2H, J=5.9 Hz), 2.09-2.04 (m, 2H), 1.00 (t, 3H, J=7.6 Hz).

Preparation of compound WLS-60a5: WLS-60a4 (12.00 g, 0.139 mol, 1.0 equiv) was taken in clean and dry 500 mL 2 neck RBF under argon atmosphere in 240 mL of ether, cooled to 0° C., added PBr₃ (15.9 mL, 0.167 mol, 1.2 equiv) dropwise for a period of 20 min. The reaction mixture was allowed to warm to room temperature and stirred for 4 h. After completion of reaction (TLC—10% MeOH:DCM) reaction mixture cool to 0° C. Then, quenched with ice water carefully (70 mL), extracted with diethyl ether (2×150 mL), washed with water (300 ml). The organic layer dried over sodium sulphate and dried under vacuum to get 11.0 g (53% yield) of WLS-60a5 as a colorless oil. The WLS-60a5 was directly used for next reaction without any further purification. ¹11 NMR (500 MHz, CDCl₃): δ in ppm=5.82 (dt, 1H, J=15.1 Hz, 6.2 Hz), 5.71-5.65 (m, 1H), 3.96 (d, 2H, J=7.6 Hz), 2.12-2.06 (m, 2H), 1.01 (m, 3H).

Preparation of compound WLS-60b: In a clean and dry two-neck 500 mL round bottom flask, WLS-60a (10.0 g, 0.116 mol, 1.0 equiv) was placed with a magnetic stirring bar and dissolved by adding DMF (150 mL). Then cool the RBF to 0° C. by using ice bath. Added sodium hydride (9.29 g, 0.232 mol, 3.0 equiv) portion wise for period of 30 min at 0° C. Stir the reaction mixture at 0° C. for 30 min. Then added WLS-60a5 (60.12 g, 0.403 mol, 3.47 equiv) dropwise by using addition funnel for period of 20 min at 0° C. and the reaction mixture was stirred for 5 h. Monitoring by TLC it showed staring material was consumed and the product was formed (TLC—50% EtOAc;Hexane, TLC charring—Phosphomolybdic acid). After completion of reaction, reaction mixture poured into ice and extracted with ethyl acetate (500 mL×2) and organic layer washed with ice cold water (500 mL×2). Organic layer dried over sodium sulphate, filtered and evaporated to dryness to get crude compound. The crude product was purified by silica gel column chromatography (100-200 mesh). The product was eluted with 15% -20% ethyl acetate:hexane. The fraction containing product was evaporated to get 18.4 g (71% yield) of WLS-60b as a yellow liquid. ¹H NMR (400 MHz, CDCl₃): δ in ppm=5.69-5.62 (m, 2H), 5.41-5.33 (m, 2H), 3.74 (dd, 4H, J=6.5, J=1.1 Hz), 3.22 (s, 4H), 2.08-2.00 (m, 4H), 0.99 (t, 6H, J=7.4 Hz).

Preparation of compound WLS-60c: WLS-60b (25.0 g, 0.112 mol, 1.0 equiv) was taken in clean and dry 2 Lit two neck RBF under argon atmosphere. Then added 350 mL of dry toluene under argon atmosphere. Added oxalyl chloride (144 mL, 1.679 mol, 14.93 equiv) dropwise using addition funnel for a period of 45 min at rt. The reaction mixture was heated to 65° C. for 72 hrs. After completion of reaction (TLC—10% MeOH:DCM; TLC charring—Phosphomolybdic acid) solvent was evaporated on rota evaporator to get crude compound. The crude compound was washed with hexane (2×500 mL), afters washing solvent was decanted and dried on high vacuum to get 31.0 g of crude WLS-60c as a brown gummy liquid. The WLS-60c was directly used for next step without any further purification. ¹H NMR (400 MHz, CDCl₃): δ in ppm=5.97-5.92 (m, 2H), 5.48-5.33 (m, 4H), 4.23 (s, 6H), 2.17-2.03 (m, 4H), 1.01 (t, 6H, J=7.4 Hz).MS: m/z calcd for Ci3H22Cl₂N2⁺IM-C11, Calculated 241.78; found 241.21.

Preparation of compound WLS-60d: WLS-60c (31.0 g, 0.112 mol, 1.0 equiv) was taken in clean and dry 2 Lit single neck RBF under argon atmosphere. Added 310 mL of DCM under argon atmosphere. Then added aq solution of KPF₆ (20.58 g, 0.112 mol, 1.0 equiv, in 124 mL of water). The reaction mixture was stirred at rt for 2.5 h. After completion of reaction (TLC—10% MeOH:DCM; TLC charring—Phosphomolybdic acid), the reaction mixture was poured into ice water (400 mL), and extracted with DCM (2×500 mL). The combined organic layer washed with water (400 mL) and dried over sodium sulphate, filtered and evaporated to dryness. The residue was dissolved in DCM (15 mL) and product was precipitate by dropwise addition of diethyl ether (2×500 mL) under stirring. The solvent was decanted and solid was dried under high vacuum. The above precipitation procedure repeat one more time to get 39.0 g (90% yield) of WLS-60d as an ash colored solid. ¹H NMR (500 MHz, CDCl₃): δ in ppm=5.92-5.86 (m, 2H), 5.42-5.36 (m, 2H), 4.11 (d, 4H, J=6.9 Hz), 4.02 (s, 4H), 2.13-2.07 (m, 4H), 1.01 (t, 6H, J=7.6 Hz). ¹⁹F NMR (500 MHz, CDCl₃): δ in ppm=-72.96, -74.48.

Preparation of compound WLS-60: WLS-60d (39.0 g, 0.101 mol, 1.0 equiv) was taken in clean and dry 1 Lit two neck RBF under argon atmosphere. Added 390 mL of Dry MeCN under argon atmosphere. Added sodium azide (9.84 g, 0.151 mol, 1.5 equiv) portion wise for the period of 10 min. The reaction mixture was stirred at rt for 3 h. After completion of reaction (TLC—5% MeOH:DCM; TLC charring—ninhydrin), reaction mixture was filtered through a pad of celite and washed with MeCN (40 mL). The organic layer was evaporated to dryness. The crude compound was washed with ether and hexane to get brown gummy liquid which was dried on high vacuum to get 32.0 g (81% yield) of WLS-60 as a brown gummy liquid. ¹H NMR (500 MHz, CDCl₃): δ in=5.89-5.84 (m, 2H), 5.44-5.40 (m, 2H), 4.04 (d, 4H, J=5.5 Hz), 3.87 (s, 4H), 2.13-2.08 (m, 4H), 1.01 (q, 6H, J=7.1 Hz). ¹⁹FNMR (500 MHz, CDCl₃): δ in ppm=-73.22 and -74.74. MS: m/z calcd for C₁₃H₂₂F₆N₅P ([M−PF₆]⁺), 248.35; found 248.80. IR (KBr pellet): N₃ (2170 cm⁻¹)

### Synthesis of 2-azido-1,3-di ((Z)-pent-2-en-1-yl)-4,5-dihydro-1H-imidazol-3-ium hexafluoro- phosphate(V) (WLS-61)

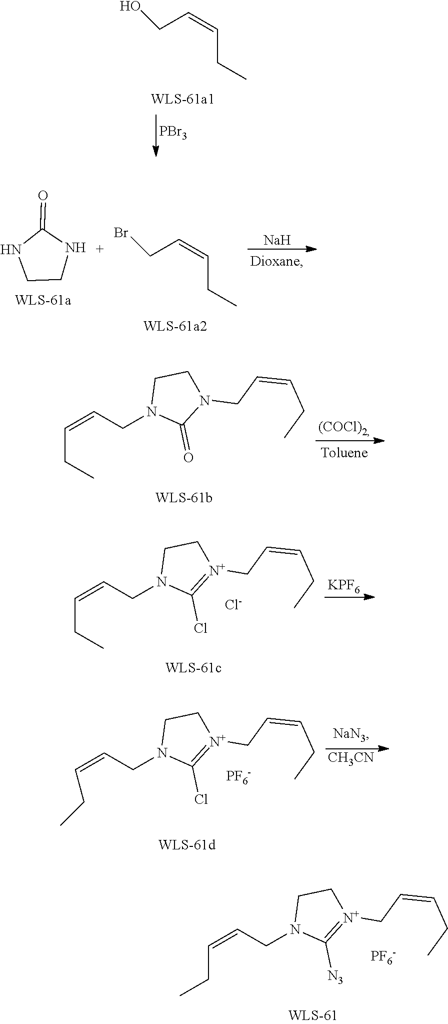

Preparation of compound WLS-61a2: WLS-61a1 (19.00 g, 0.221 mol, 1.0 equiv) was taken in clean and dry 1 L two neck RBF under argon atmosphere in 380 mL of dry ether, cooled to 0° C., added PBr₃ (25.2 mL, 0.265 mol, 1.2 equiv) dropwise for a period of 20 min. The reaction mixture was allowed to rt and stirred for 4 h. After completion of reaction (TLC—30% EtOAc:hexane; TLC charring—Phosphomolybdic acid) reaction mixture cool to 0° C. Then, quenched with ice water carefully (70 mL), extracted with diethyl ether (2×500 mL), washed with water (300 ml). The organic layer dried over sodium sulphate and dried under vacuum to get 24.0 g (73% yield) of WLS-61a2 as a colorless oil. The WLS-60a2 was directly used for next reaction without any further purification. ¹H NMR (400 MHz, CDCl₃): δ in ppm=5.74-5.66 (m, 1H), 5.63-5.57 (m, 1H), 4.00 (d, 2H, J=8.2 Hz), 2.20-2.09 (m, 2H), 1.02 (t, 3H, J=7.6 Hz).

Preparation of compound WLS-6 lb: In a clean and dry two-neck 500 mL round bottom flask, WLS-61a (5.0 g, 0.058 mol, 1.0 equiv) was placed with a magnetic stirring bar, and dissolved by adding DMF (100 mL). Then cool the RBF to 0° C. by using ice bath. Added sodium hydride (4.64 g, 0.116 mol) portion wise for period of 30 min at 0° C. Stir the reaction mixture at 0° C. for 30 min. Then added WLS-61a2 (21.63 g, 0.145 mol, 2.5 equiv) dropwise by using addition funnel for period of 30 min at 0° C. and the reaction mixture was stirred for 30 min for 0° C. and 3 h at rt. Monitoring by TLC it showed staring material was consumed and the product was formed (TLC—30% EtOAc;Hexane, TLC charring—Phosphomolybdic acid). After completion of reaction, reaction mixture poured into ice and extracted with ethyl acetate (1000 mL×2) and organic layer washed with ice cold water (1200 mL×2). Organic layer dried over sodium sulphate, filtered and evaporated to dryness to get crude compound. The crude product was purified by silica gel column chromatography (100-200 mesh). The product was eluted with 5% -10% ethyl acetate:hexane. The fraction containing product was evaporated to get 9.69 g (75% yield) of WLS-61b as a yellow liquid. ¹H NMR (400 MHz, CDCl₃): δ in ppm=5.63-5.56 (m, 2H), 5.36-5.29 (m, 2H), 3.84 (dt, 4H, J=7.1, J=0.6 Hz), 3.23 (s, 4H), 2.15-2.07 (m, 4H), 0.98 (t, 6H, J=7.5 Hz). MS: m/z calcd for C₁₃H₂₂N₂₀ ([M+H]⁺), 223.33; found 223.37.

Preparation of compound WLS-61c: WLS-61b (30.0 g, 0.135 mol, 1.0 equiv) was taken in clean and dry 2 Lit three neck RBF under argon atmosphere. Then added 300 mL of dry toluene under argon atmosphere. Added oxalyl chloride (173.6 mL, 2.024 mol, 15.0 equiv) dropwise using addition funnel for a period of 30 min at rt. The reaction mixture was heated to 65° C. for 72 hrs. After completion of reaction (TLC—10% MeOH:DCM; TLC charring—Phosphomolybdic acid) solvent was evaporated on rota evaporator to get crude compound. The crude compound was washed with hexane (2×500 mL), afters washing solvent was decanted and dried on high vacuum to get 38.0 g of crude WLS-61c as a brown gummy liquid. The WLS-61c was directly used for next step without any further purification. MS: m/z calcd for C₁₃H₂₂Cl₂N₂⁺[M⁺−Cl], Calculated 241.78; found 241.27.

Preparation of compound WLS-61d: WLS-61c (37.0 g, 0.133 mol, 1.0 equiv) was taken in clean and dry 1 Lit single neck RBF under argon atmosphere. Added 370 mL of DCM under argon atmosphere. Then added aq solution of KPF₆ (24.57 g, 0.133 mol, 1.0 equiv, in 148 mL of water). The reaction mixture was stirred at rt for 3 h. After completion of reaction (TLC—10% MeOH:DCM; TLC charring—Phosphomolybdic acid), the reaction mixture was poured into ice water (400 mL), and extracted with DCM (2×500 mL). The combined organic layer washed with water (400 mL) and dried over sodium sulphate, filtered and evaporated to dryness. The residue was dissolved in DCM (40 mL) and product was precipitate by dropwise addition of diethyl ether (1000 mL) under stirring. The solvent was decanted and solid was dried under high vacuum. The above precipitation procedure repeat one more time to get 48.0 g (93% yield) of WLS-61d as an ash colored solid. ¹H NMR (500 MHz, CDCl₃): δ in ppm=5.92-5.79 (m, 2H), 5.44-5.33 (m, 2H), 4.21 (d, 4H, J=7.6 Hz), 4.03 (s, 4H), 2.16-2.09 (m, 4H), 1.01 (t, 6H, J=7.2 Hz). ¹⁹F NMR (500 MHz, CDCl₃): δ in ppm=−73.12, −74.64. MS: m/z calcd for C₁₃H₂₂ClF₆N₂P⁺[M⁺−-Cl], Calculated 241.78; found 241.18.

Preparation of compound WLS-61: WLS-61d (48.0 g, 0.124 mol, 1.0 equiv) was taken in clean and dry 1 Lit two neck RBF under argon atmosphere. Added 480 mL of Dry MeCN under argon atmosphere. Added sodium azide (12.103 g, 0.186 mol, 1.5 equiv) portion wise for the period of 10 min. The reaction mixture was stirred at rt for 2.5 h. After completion of reaction (TLC—5% MeOH:DCM; TLC charring—ninhydrin), reaction mixture was filtered through a pad of celite and washed with MeCN (40 mL). The organic layer was evaporated to dryness. The crude compound was dissolved in DCM (100 mL) and precipitate by adding ether and hexane at −78° C., solvent was decant and solid was dried under high vacuum to get 28.0 g (57% yield) of WLS-61 as a brown solid. ¹H NMR (500 MHz, CDCl₃): 6 in=5.80-5.75 (m, 2H), 5.43-5.36 (m, 2H), 4.12 (d, 4H, J=6.9 Hz), 3.86 (s, 4H), 2.13-2.08 (m, 4H), 1.00 (q, 6H, J=7.1 Hz). ¹⁹F NMR (500 MHz, CDCl₃): δ in ppm=-73.26 and -74.78. MS: m/z calcd for C₁₃H₂₂F₆N₅P ([M−PF₆]⁺), 248.35; found 248.24. IR (KBr pellet): N₃ (2171 cm⁻¹)

### Synthesis of 2-azido-1,3-bis(2-methoxyethyl)-4,5-dihydro-1H-imidazol-3-ium hexafluoro-phosphate(V) (WLS-64)

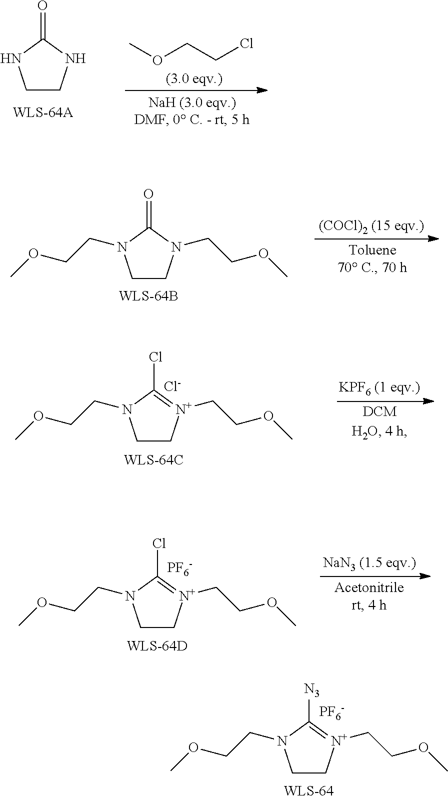

1,3-bis(2-methoxyethyl)imidazolidin-2-one (WLS-64B): To a solution of imidazolidin-2-one (WLS-64A) (20 g, 0.23 mol, 1.0 equiv) in DMF (20 mL) was added sodium hydride (28 g, 0.69 mol., 3.0 equiv) portion-wise at 70° C. over a period of 40 min, stirred at the same temperature for 2 h. Then a solution of 2-chloroethyl methyl ether (63.9 mL, 0.69 mol, 3.0 equiv) in DMF (60 mL) was added dropwise over a period of 30 mins. Above mixture was stirred at 70° C. for 3 h. Progress of the reaction was monitored by TLC. Above reaction was diluted with ice water (300 mL) and extracted with ethyl acetate (2×500 mL). Combined organic layers was washed with cold brine solution (3×100 mL) dried over Na₂SO₄ and concentrated under reduced pressure. The crude was purified by column chromatography over silica gel (60-120 mesh) eluted in 80% EA/Hexane to give 1,3-bis(2-methoxyethyl)imidazolidin-2-one (WLS-64B) as a colourless oil (29 g, 62%). 1H NMR (400 MHz, CDCl₃): δ in ppm=3.52 (t, 4H, J=5.2 Hz)), 3.42 (s, 4H), 3.37 (t, 4H, J=5.3 Hz), 3.35 (s, 6H). MS (ESI) 203.21 (M+1)⁺.

2-chloro-1,3-bis(2-methoxyethyl)-4,5-dihydro-1H-imidazol-3-ium chloride (WL S-64 C) : To a cool solution of (WLS-64B) (15 g, 0.074 mol, 1.0 equiv) in toluene (150 mL) was added oxalyl chloride (95 mL, 1.1138 mol., 15.0 equiv) dropwise over a period of 25 min under argon atmosphere. Above mixture was stirred at 70 oC for 72 h. Progress of the reaction was monitored by TLC. Above reaction mixture was concentrated under reduced pressure to afford a crude compound. The crude was treated with n-hexane (2×100 mL) and 40% EA/Hexane (3×100 mL) at 0 oC. A solid precipitation was observed at 0° C., then solvent was decant and the compound was dried under vacuum to afford a brownish gummy syrup (17 g, crude) which was used for the next step without further purification. 1H NMR (400 MHz, CDCl₃): δ in ppm=4.38 (d, 4H, J=18.6 Hz), 3.89 (t, 4H, J=4.9 Hz) 3.66 (t, 4H, J=4.9 Hz), 3.39 (s, 6H). MS (ESI) 221.19 (M−Cl)+.

2-chloro-1,3-bis(2-methoxyethyl)-4,5-dihydro-1H-imidazol-3-ium hexafluorophosphate(V) (WLS-64D): To a stirred solution of (WLS-64C) (14 g, 0.0544 mol, 1.0 equiv) in DCM (140 mL) was added a solution of KPH₆ (10 g, 0.0544 mol., 1.0 equiv) in water (70 mL) dropwise over a period of 30 mins at rt. Above reaction mixture was stirred at rt for 4 h. Progress of the reaction was monitored by TLC. Then the mixture was filtered through a celite bed washed with DCM (3×100 mL). Organic layer was washed with water (2×100 mL) dried over Na2SO4, filtered and evaporated to dryness. The residue was dissolved in DCM (15 mL) and then added diethyl ether (125 mL), cool to −78 oC. The solid precipitated was filtered and washed with ether (2×50 mL) and dried under vacuum to yield a brown solid (25 g, 64%). 1H NMR (500 MHz, CDCl₃): δ in ppm=4.21 (td, 4H, J=10.8 Hz, 5.3 Hz), 3.78 (m, 4H), 3.62 (q, 4H, J=5.5 Hz), 3.38 (d, 6H, J=2.8 Hz). MS (ESI) 221.18 (M−PF6)+.

2-azido-1,3-bis(2-methoxyethyl)-4,5-dihydro-1H-imidazol-3-ium hexafluorophosphate(V) (WLS-64): To cool solution of (WLS-64D) (12.5 g, 0.034 mol, 1.0 equiv) in acetonitrile (125 mL) was added sodium azide (3.32 g, 0.051 mol., 1.5 equiv) portion wise over a period of 20 mins under N2 atmosphere. Above reaction mixture was stirred at rt for 4 h. Progress of the reaction was monitored by TLC. Then the mixture was filtered through a celite bed, the bed was washed with acetonitrile (2×80 mL). The filtrate was evaporated under vacuum to afford a crude mass. The residue was again dissolved in DCM (25 mL) and then added diethyl ether (150 mL) cool to −60 oC and stirred for 40 min. The solid was precipitated which was filtered and washed with ether (2×50 mL) and dried under high vacuum to afford a brownish gummy mass(11 g, 86%). 1H NMR (500 MHz, CDCl₃): δ in ppm=3.98 (s, 4H), 3.64 (q, 4H, J=4.4 Hz), 3.59 (dt, 4H, J=14.2 Hz, 5.3 Hz), 3.40 (d, 6H, J=8.3 Hz). MS (ESI) 228.25 (M−PF6)+. 19F NMR (500 MHz, CDCl₃): δ in ppm=−72.95 and −74.46. IR (KBr pellet): N3 (2173 cm-1).

### Synthesis of azido-1-methyl-4-(6-(2,2,2-trifluoroacetamido)hexyl)-3,4-dihydro-2H-Dyrrol-1-ium hexa-fluorophosphate(V) (WLS-66)

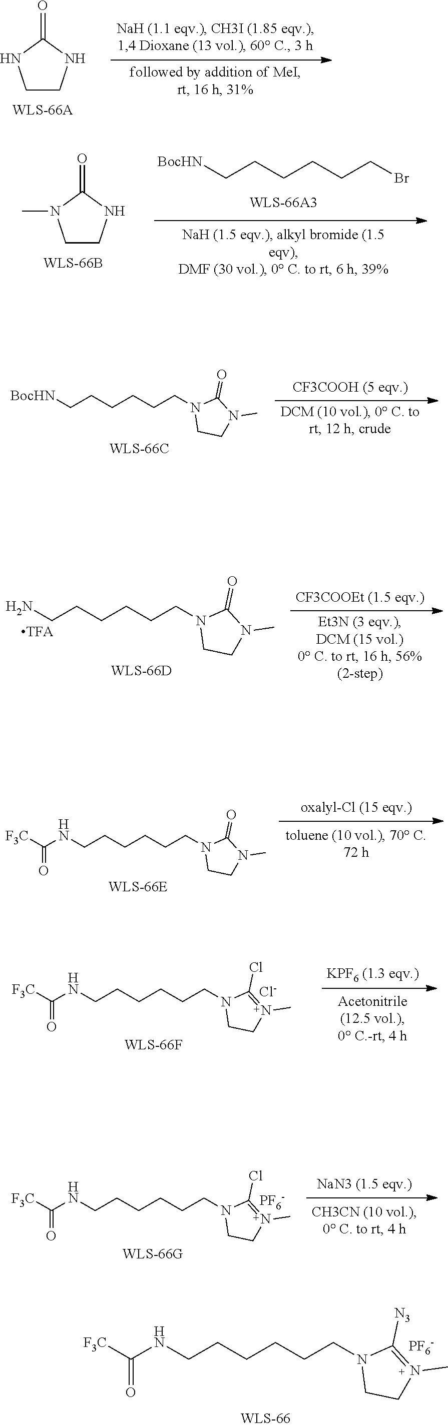

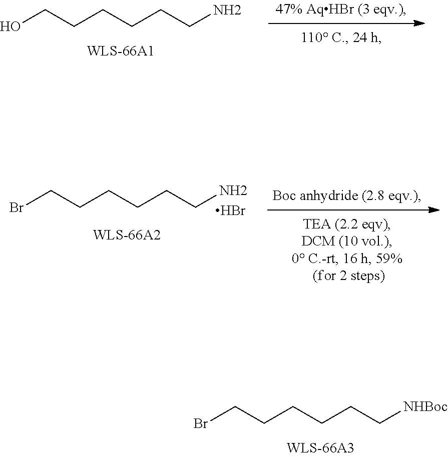 [SMILES: NCCCCCCO ; NCCCCCCBr ; Br ; CC(C)(C)OC(=O)NCCCCCCBr]

### Synthesis of (WLS-66B): To a solution of (WLS-66A) (50 g, 0.58 mol, 1.0 equiv) in 1,4-dioxane (650 mL, 13 vol.) was added sodium hydride (27.18 g, 0.67 mol., 1.17 equiv) portion-wise at 0° C. over a period of 30 min, and further stirred at 65° C. for 3 h. Then lodomethane (63.8 mL, 1.07 mol, 1.8 equiv) was added dropwise over a period of 45 mins at 0° C. The reaction mixture was allowed to rt and stirred for 16 h. Progress of the reaction was monitored by TLC. Above reaction was filtered through a celite bed, which was washed with DCM (2×100 mL). Filtrate was concentrated under reduced pressure to afford a crude compound which was purified by column chromatography over silica gel (230-400 mesh) eluted in 2% MeOH/DCM to get (WLS-66B) as an off-white solid (18 g, 31%). ¹H NMR (500 MHz, CDCl₃): δ in ppm=5.10 (s, 1H), 3.41 (m, 4H), 2.79 (s, 3H). MS (ESI) 101.01 (M+1)⁺.

### Synthesis of (WLS-66C): To a stirred solution of (WLS-66B) (14 g, 0.1398 mol, 1.0 equiv) in DMF (350 mL, 25 vol.) was added sodium hydride (60%) (8.38 g, 0.2097 mol., 1.5 equiv) portion wise over a period of 30 min under argon atmosphere at 0° C. To the reaction mixture a solution of alkyl bromide (58.71 g, 0.2097 mol, 1.5 equiv) in DMF (70 ml, 5 vol.) was added dropwise over a period of 1 h. Then the mixture was allowed to stir for another 3 h. Progress of the reaction was monitored by TLC. Above reaction was diluted with ice water (300 mL) and extracted with ethyl acetate (3×400 mL), dried over Na₂SO₄ and concentrated under reduced pressure. The crude was purified by column chromatography over silica-gel (230-400 mesh) eluted with 2% MeOH/DCM to afford (WLS-66C) as a pale yellow oil (16.5 g, 39%). ¹H NMR (500 MHz, CDCl₃): δ in ppm=4.55 (s, 1H), 3.27 (s, 4H), 3.17 (t, 2H, J=7.2 Hz), 3.09 (t, 2H, J=5.9 Hz), 2.78 (s, 3H), 1.50 (q, 4H, J=7.3 Hz), 1.44 (s, 9H), 1.32 (m, 4H). MS (ESI) 300.33 (M+1)⁺.

Synthesis of (WLS-66D): To a stirred solution of (WLS-66C) (18 g, 0.06012 mol, 1.0 equiv) in DCM (180 mL, 10 vol.) was added trifluoroacetic acid (23.1 mL, 0.3006 mol., 5.0 equiv) dropwise at 0° C. Above reaction mixture was stirred at rt for 6 h. Progress of the reaction was monitored by TLC. Then the reaction mixture was evaporated under reduced pressure co-distilled with toluene and dried to afford a yellowish gummy mass (20 g, crude) which was used for the next step without further purification. ¹H NMR (400 MHz, CDCl₃): δ in ppm=7.65 (d, 2H, J=36.6 Hz), 3.21 (s, 4H), 3.04 (t, 2H, J=7.1 Hz), 2.77 (m, 2H), 2.63 (s, 3H), 1.52 (m, 2H), 1.42 (m, 2H), 1.28 (m, 4H). MS (ESI) 200.25 (M+1)⁺.

Synthesis of (WLS-66E): To a cool stirred solution of (WLS-66D) (20 g, 0.06410 mol, 1.0 equiv) in DCM (300 mL) was added triethylamine (26.87 mL, 0.1923 mol., 3.0 equiv) dropwise over a period of 30 mins. Then ethyl trifluoroacetate (11.48 mL, 0.09615 mol., 1.5 equiv) was added dropwise over a period of 15 mins. Above reaction mixture was stirred at rt for 16 h. Progress of the reaction was monitored by TLC. Above reaction was diluted with ice water (100 mL) and extracted with DCM (3×100 mL), then dried over Na₂SO₄ and concentrated under reduced pressure. The crude compound was purified by column chromatography over basic silica-gel (100-200 mesh) eluted with 90% EA/Hexane to afford (WLS-66E) as an off-white solid (9.9 g, 56%, for 2 steps). ¹H NMR (400 MHz, CDCl₃): δ in ppm=7.43 (s, 1H), 3.33 (q, 2H, J=6.5 Hz), 3.29 (t, 4H, J=3.6 Hz), 3.20 (t, 2H, J=6.8 Hz), 2.77 (s, 3H), 1.59 (m, 2H), 1.51 (m, 2H), 1.41 (m, 2H), 1.30 (m, 2H). MS (ESI) 296.3 (M+1)⁺.

Synthesis of (WLS-66F): To a cool solution of (WLS-66E) (12 g, 0.0405 mol, 1.0 equiv) in toluene (120 mL, 10 vol.) was added oxalyl chloride (52.6 mL, 0.6089 mol., 15 equiv) dropwise over a period of 20 min under argon atmosphere. Above mixture was stirred at 70° C. for 72 h. Progress of the reaction was monitored by TLC. Above reaction mixture was concentrated under reduced pressure to afford a crude compound which was treated with diethyl ether (2×60 mL), solvent was decant and then was dried under vacuum to afford (WLS-66F) as a brown mass (16 g, crude) which was used for the further step without further purification. ¹H NMR (500 MHz, CDCl₃): δ in ppm=11.30 (s, 1H), 4.30 (t, 4H, J=6.9 Hz), 3.66 (m, 4H), 3.32 (d, 3H, J=5.5 Hz), 1.80 (m, 4H), 1.42 (m, 4H). MS (ESI) 314.31 (M+1)⁺.

Synthesis of (WLS-66G): To a cool stirred solution of (WLS-66F) (5 g, 0.0142 mol, 1.0 equiv) in acetonitrile (62.5 mL) was added solid KPH6 (3.41 g, 0.0185 mol., 1.3 equiv) portion wise over a period of 10 mins. Above reaction mixture was stirred at rt for 4 h. Progress of the reaction was monitored by TLC. Then the mixture was filtered through a celite bed washed with acetonitrile (2×20 mL). Filtrate was evaporated under reduced pressure to afford a crude compound. The residue was again dissolved in acetonitrile (5 mL) and then treated with diethyl ether (60 mL) at −78° C., solvent was decant, dried under vacuum to get (WLS-66G) as a brownish gummy mass (4 g, crude). ¹H NMR (500 MHz, CDCl₃): δ in ppm=4.13 (s, 4H), 3.62 (m, 4H), 3.26 (s, 3H), 1.64 (m, 4H), 1.41 (d, 4H, J=15.8 Hz). MS (ESI) 314.26 (M+1)⁺.

Synthesis of (WLS-66G): To a cool stirred solution of (WLS-66G) (48 g, 0.1044 mol, 1.0 equiv) in acetonitrile (480 mL) was added sodium azide (10.18 g, 0.1566 mol., 1.5 equiv) portion-wise over a period of 20 min. Above reaction mixture was stirred at rt for 4 h. Progress of the reaction was monitored by TLC. Then the mixture was filtered through a celite bed washed with acetonitrile (2×100 mL). The filtrate was evaporated under vacuum to afford a crude compound. The residue was dissolved in acetonitrile (25 mL) and then added diethyl ether (200 mL), cool to −78° C. The solid was not precipitate out, solvent was decant and dried under vacuum to yield a brownish gummy liquid (44 g). ¹H NMR (500 MHz, DMSO-D6): δ in ppm=9.43 (s, 1H), 3.81 (ddd, 4H, J1=23.1 Hz, J2=15.5 Hz, J3=4.5 Hz)), 3.34 (t, 4H, J=6.9 Hz), 3.13 (s, 3H), 1.51 (m, 4H), 1.28 (s, 4H). MS (ESI) 321.34 (M+1)⁺. ¹⁹F NMR (500 MHz, CDCl₃): 6 in ppm=−69.325 and −70.837. IR (KBr pellet): N3 (2169.53 cm⁻¹).

### Synthesis of (WLS-66A2): To (WLS-66A1) (40 g, 0.3413 mol, 1.0 equiv) was added aqueous HBr (47%) (118 mL, 1.0239 mol., 3.0 equiv) drop-wise over a period of 30 mins at 0° C. Above reaction mixture was stirred at 110° C. for 24 h. Progress of the reaction was monitored by TLC. Solvent was evaporated under vacuum to afford a crude compound (WLS-66A2) as a pale yellow semi solid (80 g) which was used for the next step without further purification. ¹H NMR (500 MHz, CDCl₃)): δ in ppm=7.90 (s, 2H, −NH2), 3.42 (q, 2H, J=7.1 Hz), 3.09 (q, 2H, J=6.4 Hz), 1.87 (m, 4H), 1.51 (m, 4H). MS (ESI) 180.15 (M, M+2)⁺.

Synthesis of (WLS-66A3): To a cool stirred solution of (WLS-66A2) (80 g, 0.3065 mol, 1.0 equiv) in DCM (800 mL, 10 vol.) was added triethylamine (95.5 mL, 0.6743 mol., 2.2 equiv) dropwise over a period of 20 mins. Then Boc anhydride (187 ml, 0.8582 mol., 2.8 equiv) was added dropwise over a period of 45 min. the mixture was allowed to rt and stirred for 16 h. Progress of the reaction was monitored by TLC. The mixture was diluted with DCM (500 mL) and washed with water (4×200 mL). The organic layer was dried over Na₂SO₄ and evaporated under vacuum to afford a crude compound. The residue was purified by column chromatography over silica gel (230-400 mesh) eluted with 8% EA/Hexane to give (WLS-66A3) as a pale-yellow syrup (57 g, 59%, for 2 steps). ¹H NMR (400 MHz, CDCl₃): δ in ppm=4.55 (s, 1H, −NH), 3.40 (t, 2H, J=6.8 Hz), 3.11 (q, 2H, J=6.4 Hz), 1.83 (m, 2H), 1.49 (m, 13H), 1.34 (m, 2H). MS (ESI) 280.24 (M+)⁺.

### Synthesis of 2-azido-1-((4Z,7Z)-deca-4,7-dien-1-yl)-3-methyl-4,5-dihydro-1H-imidazol-3-ium hexafluorophosphate (V) (WV-RA-016A)

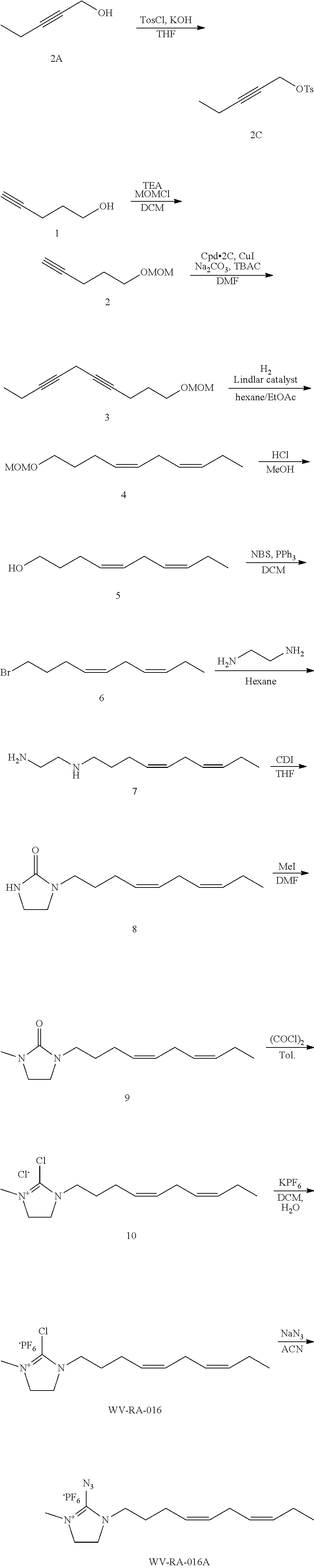

Preparation of compound 2C: To a solution of compound 2A (76 g, 903.51 mmol) in THF (1500 mL) was added TosCl (206.70 g, 1.08 mol) and KOH (76.04 g, 1.36 mol). The mixture was stirred at 0° C. for 4 hr. TLC indicated compound 2A was consumed completely and one new spot formed. The reaction mixture was filtered off the insoluble matter, and the filtrate was concentrated under reduced pressure to give a residue. The residue was purified by column chromatography (SiO₂, Petroleum ether/Ethyl acetate=1/0 to 2/1). Compound 2C (210 g, 97.53% yield) was obtained as a yellow oil. TLC: Petroleum ether: Ethyl acetate=5:1, Rf=0.4.

Preparation of compound 2: To a solution of compound 1 (72.8 g, 865.47 mmol) in DCM (800 mL) was added DIEA (257.27 g, 1.99 mol) at 0° C., followed by dropwise addition of MOMC1 (143.89 g, 1.79 mol). The mixture was stirred at 0° C. for 2 hr under N₂. TLC indicated compound 1 was consumed and one new spot formed. Saturated NH₄Cl solution (1000 mL) was added, the layers were separated and the aqueous mixture was further extracted with DCM (2 * 500 mL). The combined organic fractions were dried (Na₂SO₄) and the solvent was removed in vacuo. Compound 2 (70 g, 63.11% yield) was obtained as a colorless oil. ¹HNMR (400 MHz, CHLOROFORM-d) δ=4.61 (s, 2H), 3.61 (t, J=6.2 Hz, 2H), 3.35 (s, 3H), 2.30 (dt, J=2.7, 7.0 Hz, 2H), 1.94 (t, J=2.6 Hz, 1H), 1.80 (quin, J=6.6 Hz, 2H). TLC: Petroleum ether: Ethyl acetate=2:1, Rf=0.6.

Preparation of compound 3: Tetrabutylammonium;chloride (33.83 g, 121.71 mmol), disodium;carbonate (64.50 g, 608.57 mmol) and iodocopper (77.27 g, 405.72 mmol) each finely ground and anhydrous, were suspended in dry DMF (1000 mL) at 0° C. with stirring. Subsequently, compound 2 (52 g, 405.72 mmol) was added all at once and kept stirring for 20 min. Compound 2C (116.02 g, 486.86 mmol) was added dropwise and the suspension was stirred at 40° C. under N2 for 12 h. TLC indicated compound 2 was consumed and one main new spot formed. The reaction mixture was diluted with sat.NH₄C1 500 mL, H₂O 500 mL and extracted with ethyl acetate (500 mL * 3). The combined organic layers were washed with sat.brine 500*2 mL, dried over Na₂SO₄, filtered and concentrated under reduced pressure to give a residue. The residue was purified by column chromatography (SiO₂, Petroleum ether/Ethyl acetate=1/0 to 5/1). Compound 3 (24 g, 30.45% yield) was obtained as a yellow oil. ¹HNMR (400 MHz, CHLOROFORM-d) δ=4.62 (s, 2H), 3.60 (t, J=6.3 Hz, 2H), 3.36 (s, 3H), 3.11 (quin, J=2.3 Hz, 2H), 2.28 (tt, J=2.3, 7.0 Hz, 2H), 2.17 (tq, J=2.3, 7.5 Hz, 2H), 1.77 (quin, J=6.7 Hz, 2H), 1.11 (t, J=7.5 Hz, 3H). TLC: Petroleum ether: Ethyl acetate=5:1, Rf=0.8.

Preparation of compound 4: To a solution of compound 3 (11 g, 56.62 mmol) in the mixture solvent of hexane (90 mL) and EtOAc (30 mL) was added quinoline (146.27 mg, 1.13 mmol) and LINDLAR CATALYST (11.69 g, 5.66 mmol, 10% purity) under H₂ atmosphere (15 psi). The mixture was stirred at 15° C. for 12 hr. TLC indicated compound 3 was consumed completely and two new spots formed. The reaction mixtures of two batches were filtered and concentrated under reduced pressure to give a residue. The residue was purified by column chromatography (SiO₂, Petroleum ether/Ethyl acetate=1/0 to 10/1). Compound 4 (17 g, 75.70% yield) was obtained as a colorless oil. ¹HNMR (400 MHz, CHLOROFORM-d) δ=5.57-5.17 (m, 4H), 4.69-4.58 (m, 2H), 3.56-3.50 (m, 2H), 3.38-3.36 (m, 3H), 2.86-2.65 (m, 2H), 2.22-2.05 (m, 4H), 1.74-1.60 (m, 2H), 1.02-0.92 (m, 3H). TLC: Petroleum ether: Ethyl acetate=5:1, Rf=0.8.

Preparation of compound 5: HCl (6 M, 142.88 mL) was added to a stirred solution of compound 4 (17 g, 85.73 mmol) in MeOH (150 mL). The mixture was stirred at 70° C. for 2 h. TLC indicated compound 4 was consumed completely and one new spot formed. The reaction mixture was quenched with 1M NaOH to pH-7, and then was extracted with EtOAc (3×200 mL) and the combined organic layers were washed with brine (200 mL), dried (Na₂SO₄), and concentrated. The residue was purified by column chromatography (SiO₂, Petroleum ether/Ethyl acetate=1/0 to 1/1). Compound 5 (9 g, 68.06% yield) was obtained as a yellow liquid. TLC: Petroleum ether: Ethyl acetate=5:1, Rf=0.3.

Preparation of compound 6: NBS (20.77 g, 116.69 mmol) was added portionwise to an ice-cooled solution of PPh₃ (30.61 g, 116.69 mmol) in DCM (300 mL) under N2. The mixture was stirred at 0° C. for 15 min and then a solution of compound 5 (9 g, 58.35 mmol) in DCM (50 mL) was slowly added. The mixture was stirred in an ice bath for 2 h and for 3 h more at 15° C. TLC (Petroleum ether: Ethyl acetate=5:1, Rf=0.9) indicated compound 5 was consumed completely and one new spot formed. The reaction mixture was quenched with H₂O (100 mL) and extracted with CH₂Cl₂ (3×200 mL). The combined organic layers were concentrated under vacuum. The residue was purified by column chromatography (SiO₂, Petroleum ether/Ethyl acetate=1/0 to 3/1). Compound 6 (10 g, 46.05 mmol, 78.93% yield) was obtained as a colorless liquid. ¹HNMR (400 MHz, CHLOROFORM-d) δ=5.58-5.21 (m, 4H), 3.47-3.37 (m, 2H), 2.88-2.68 (m, 2H), 2.23 (td, J=7.4, 14.9 Hz, 2H), 2.13-2.06 (m, 2H), 1.98-1.89 (m, 2H), 1.04-0.92 (m, 3H). TLC: Petroleum ether: Ethyl acetate=5:1, Rf=0.9.

Preparation of compound 7: To a solution of compound 6 (9.5 g, 43.75 mmol) in Hexane (50 mL) was added ethane-1,2-diamine (78.38 g, 1.30 mol) at 0° C. The mixture was stirred at 0-15° C. for 5 hr. TLC indicated compound 6 was consumed completely and one new spot formed. The mixture was concentrated under reduced pressure. Compound 7 (8.59 g, crude) was obtained as a colorless oil. TLC (Petroleum ether: Ethyl acetate=5:1, Rf=0).

Preparation of compound 8: To a solution of compound 7 (8.59 g, 43.75 mmol) and CDI (7.09 g, 43.75 mmol) in THF (90 mL) was stirred at 15° C. for 12 hr. TLC indicated compound 7 was consumed completely and one new spot formed. The reaction mixture was concentrated under reduced pressure to give a residue. The residue was purified by column chromatography (SiO₂, Petroleum ether/Ethyl acetate=1/0 to 1/0) to get 4.4 g crude. Then the crude was purified by reversed-phase HPLC (column: Welch Xtimate C18 250*70 mm#10 um; mobile phase: [water (10 mM NH₄HCO3)-ACN];B%: 45%-65%,20min). Compound 8 (2.5 g, 25.70% yield) was obtained as a yellow oil. ¹HNMR (400 MHz, CHLOROFORM-d) δ=5.53-5.18 (m, 4H), 3.41 (s, 4H), 3.25-3.13 (m, 2H), 2.82-2.62 (m, 2H), 2.14-2.05 (m, 3H), 2.02 (br d, J=3.6 Hz, 1H), 1.65-1.50 (m, 2H), 1.02-0.89 (m, 3H). TLC: Petroleum ether: Ethyl acetate=0:1, Rf=0.25.

Preparation of Compound 9

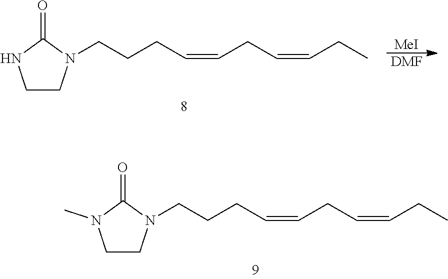

To a solution of compound 8 (2.1 g, 9.45 mmol) in DMF (20 mL) was added NaH (1.13 g, 28.34 mmol, 60% purity) at 0° C. and the reaction stirred for 0.5 h, then added MeI (6.70 g, 47.23 mmol) to the above reaction mixture, and stirred at 15° C. for 2 h. TLC indicated compound 8 was consumed and one new spot formed. The reaction mixture was quenched by addition H₂O (50 mL) at 15° C., and extracted with ethyl acetate (30 mL * 3). The combined organic layers were dried over anhydrous Na₂SO₄, filtered and concentrated under reduced pressure to give a residue. The residue was purified by column chromatography (SiO₂, Petroleum ether/Ethyl acetate=1/0 to 0/1). Compound 9 (2.23 g, 9.44 mmol, 100.00% yield) was obtained as a colorless oil. ¹HNMR (400 MHz, CHLOROFORM-d) δ=5.61-5.13 (m, 4H), 3.31-3.25 (m, 4H), 3.23-3.15 (m, 2H), 2.82-2.70 (m, 5H), 2.15-1.98 (m, 4H), 1.63-1.50 (m, 2H), 1.02-0.92 (m, 3H). TLC: Petroleum ether: Ethyl acetate=0:1, Rf=0.4.

Preparation of compound 10: A mixture of compound 9 (2 g, 8.46 mmol) in Tol. (20 mL) was degassed and purged with N₂ for 3 times, and then to the mixture was added (COCl)₂ (10.74 g, 84.62 mmol) and stirred at 65° C. for 24 hr under N₂ atmosphere. TLC showed the reaction was completed, staring material was consumed, desired product was obtained. LCMS showed the desired mass was detected. Then the mixture was concentrated in vacuo. Compound 10 (2.46 g, crude, Cl) was obtained as a black brown oil. LCMS (M +H⁺): 255.2 TLC: Petroleum ether: Ethyl acetate=0:1, R_{f}=0.

Preparation of compound WV-RA-016: To a solution of compound 10 (2.4 g, 8.24 mmol, Cl) in CAN (30 mL) was added potassium;hexafluorophosphate (1.52 g, 8.24 mmol). The mixture was stirred at 15° C. for 2 hr. A large number of solids are precipitated form the reaction mixture. The reaction mixture was filtered, and the filter cake was washed with DCM (30 mL * 2), the organic layer was concentrated. The crude was diluted with EtOAc 20 mL and extracted with H₂O (10 mL * 3). The organic layers were concentrated under reduced pressure to give a residue. Compound WV-RA-016 (3.3 g, 97.70% yield, PF6) was obtained as a brown solid. ¹HNMR (400 MHz, DMSO-d₆) δ=5.59-5.14 (m, 4H), 3.23-3.18 (m, 4H), 3.08-2.98 (m, 2H), 2.79-2.66 (m, 2H), 2.62 (s, 3H), 2.10-1.98 (m, 4H), 1.52-1.40 (m, 2H), 0.96-0.87 (m, 3H). ¹⁹F NMR (376 MHz, DMSO-d₆) δ −69.19 (s, 1F), −71.08 (s, 1F). ³¹P NMR (162 MHz, DMSO-d₆) δ −135.42 (s, 1P), -139.81 (s, 1P), −144.19 (s, 1P), −148.59 (s, 1P), −152.98 (s, 1P). LCMS (M +H⁺): 255.2, LCMS purity: 97.77% purity.

Preparation of compound WV-RA-016 A: To a solution of WV-RA-016 (100 mg, 249.52 umol PF6) in ACN (3 mL) was added NaN₃ (20 mg, 307.65 umol) . The mixture was stirred at 0° C. for 30 min. LCMS showed the de-N₂ mass was detected. The mixture was filtered through a celite pad and the filtrate was concentrated in vacuo. The residue was dissolved in 2 mL CH₃CN and the solution was poured into ether to form the precipitate, filtered, the solid was desired and the organic phase was adjusted with 2 M NaOH to pH˜13, then quenched by addition NaC10 (aq.) 20 mL. Compound WV-RA-016A (80 mg, crude, PF6) was obtained as a brown oil. ¹HNMR (400 MHz, DMSO-d₆) δ=5.57-5.06 (m, 3H), 3.80-3.48 (m, 4H), 3.39-3.30 (m, 3H), 3.27-3.15 (m, 2H), 2.87-2.72 (m, 3H), 2.12-1.90 (m, 4H), 1.64-1.37 (m, 2H), 1.00-0.83 (m, 4H). ¹⁹FNMR (376 MHz, DMSO-d₆) δ −69.22 (s, 1F), -71.11 (s, 1F). 31PNMR (162 MHz, DMSO-d₆) δ −135.42 (s, 1P), −139.81 (s, 1P), −144.19 (s, 1P), −148.59 (s, 1P), -152.98 (s, 1P). LCMS (M−N₂): 234.3.

### Synthesis of 2-azido-1-dodecyl-3-methyl-4,5-dihydro-1H-imidazol-3-ium hexafluorophosphate (WV-DL-045)

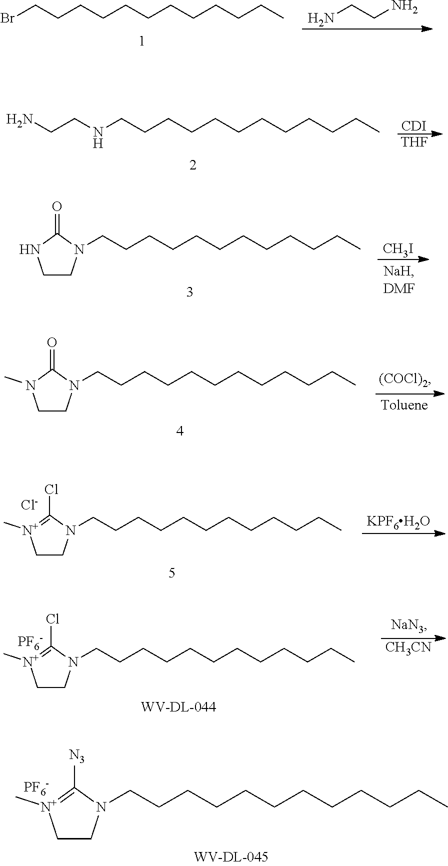

Preparation of compound 2: In a one-neck round bottom flask, ethane-1,2-diamine (337.59 g, 5.62 mol) was placed with a magnetic stirring bar, and compound 1 (50 g, 200.62 mmol) was added slowly at 0° C. After finishing the addition, the reaction mixture was warmed to 25° C., and left undisturbed for an additional 1h. 300 mL of hexane was added into the reaction mixture, which was stirred vigorously for 12 h at 25° C. LCMS showed the reaction was completed, staring material was consumed and the product was obtained, the hexane layer was decanted and dried under reduced pressure to give compound 2 (123 g) crude as colorless oil. LCMS: (M-41⁺) 229.2.

Preparation of compound 3: Two batches in parallel. To a solution of compound 2 (61.5 g, 269.25 mmol) and CDI (43.66 g, 269.25 mmol) in THF (630 mL) was stirred at 15° C. for 12 hr. TLC showed the reaction was completed, starting material was consumed and the product was obtained. The crude reaction mixture (126 g scale) was combined to another two batch crude product (123 g scale) and (84 g scale) for further purification. The combined crude product was purified by column chromatography on a silica gel eluted with petroleum ether: ethyl acetate (from 10/1 to 1/12) to give product 3 (95 g, 65.09% yield) as a white solid. TLC (Ethyl acetate : Methanol=10: 1) R_{f1}=0.50.

Preparation of compound 4: Six batches in parallel. To a solution of compound 3 (40 g, 157.23 mmol) in DMF (650 mL) was added NaH (7.55 g, 188.67 mmol, 60% purity) at 0° C. and the reaction stirred for 0.5 h, Then added CH₃I (66.95 g, 471.68 mmol) to the above reaction mixture, and stirred at 25° C. for 3 h. TLC showed the reaction was completed, starting material was consumed and the product was obtained. The reaction mixture was quenched by addition H₂O (1000 mL) at 25° C., and extracted with Ethyl acetate (1000 mL * 3). The combined organic layers were dried over anhydrous Na₂SO₄, filtered and concentrated under reduced pressure to give a residue. The residue was purified by column chromatography (SiO₂, Petroleum ether/Ethyl acetate=20/1 to 1/2) to give product 4 (232 g, crude) as yellow oil. ^{1H} NMR (400 MHz, CHLOROFORM-d) δ=3.25-3.17 (m, 4H), 3.09 (t, J=7.3 Hz, 2H), 2.70 (d, J=1.6 Hz, 3H), 1.45-1.36 (m, 2H), 1.28-1.14 (m, 19H), 0.85-0.76 (m, 3H). TLC (Petroleum ether : Ethyl acetate=0: 1) R_{f1}=0.5.

Preparation of compound 5: A mixture of compound 4 (30 g, 111.76 mmol, 1 eq.) in Tol.(250 mL) was degassed and purged with N2 for 3 times, and then to the mixture was added oxalyl chloride (212.78 g, 1.68 mol, 146.75 mL, 15 eq.) and stirred at 65° C. for 72 hr under N2 atmosphere. LCMS showed the reaction was completed, staring material was consumed, the desired product was obtained. Then the mixture was concentrated in vacuo. The white solid was washed by cooled EtOAc (100 mL*2), and then the solid was concentrated in vacuo, to give product 5 (20 g, crude) as a white solid. LCMS: 287.3.

Preparation of compound WV-DL-044: To a solution of compound 5 (8 g, 24.74 mmol) in DCM (46 mL) and H₂O (26 mL) was added potassium hexafluorophosphate (4.55 g, 24.74 mmol) at 25° C. The reaction mixture was stirred at 25° C. for 1 h. TLC showed the reaction was completed, starting material was consumed, and the desired product was obtained. The filtrate was washed with H₂O (10 mL * 2), and the white solid was desired compound. To give product WV-DL-044 (6.5 g, 60.69% yield, F6P) as a white solid. The product was combined with another two batches product (2.5 g), and (2.55 g) for analysis and delivery. Finally, 11.5 g of product was obtained. TLC (Petroleum ether : Ethyl acetate=0: 1) R_{f}=0.0.

Preparation of Lipid Azide WV-DL-045: 2.2 g WV-DL-044 and 495mg NaN₃ were added to a round bottom flask. Dry ACN was added forming a suspension and stirred 2.5 hr at room temperature. The reaction mixture was filtered through a pad of celite and washed with CAN. The filtrate was dried on rotovap and was then redissolved in a minimal amount ACN and the solution was precipitated with diethyl ether to afford 1.75 g of fluffy white solid. ¹H NMR (600 MHz, Chloroform-d) δ 3.87 (dd, J=12.1, 8.1 Hz, 1H), 3.81-3.75 (m, 1H), 3.29 (t, J=7.8 Hz, 1H), 3.12 (s, 2H), 1.57-1.50 (m, 1H), 1.22 (s, 3H), 1.19 (s, 6H), 0.84-0.78 (m, 2H). ¹³C NMR (151 MHz, CDCl₃) δ 154.76, 77.29, 77.07, 76.86, 49.38, 47.03, 46.52, 33.13, 31.90, 29.61, 29.61, 29.54, 29.42, 29.34, 29.05, 26.97, 26.47, 22.68, 14.11.

### Synthesis of 2-azido-1-hexadecyl-3-methyl-4,5-dihydro-1H-imidazol-3-ium hexafluorophosphate(V) (WL S-41)

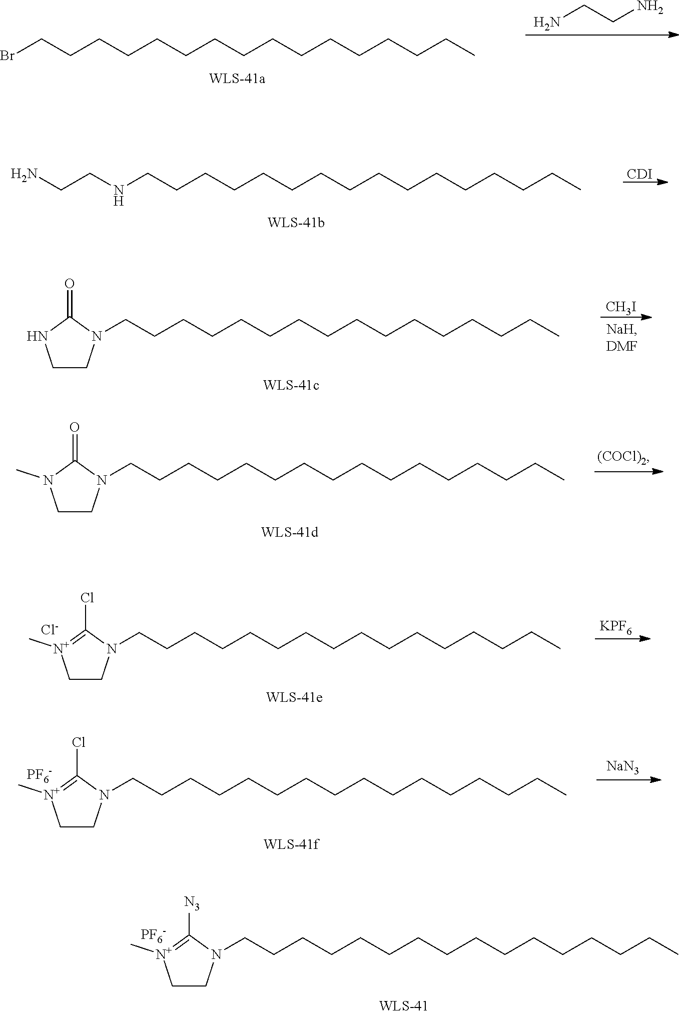

Preparation of compound WLS-41b: In a clean and dry two-neck 1 Lit round bottom flask, ethane-1,2-diamine (306 mL, 4.585 mol, 28.0 equiv) was placed with a magnetic stirring bar, and compound WLS-41a (50 g, 0.164 mol, 1.0 equiv) was added dropwise at 0° C. by using addition funnel. After finishing the addition, the reaction mixture was warmed to 25° C., and left undisturbed for an additional 1 h. Then, 300 mL of hexane was added into the reaction mixture and stirred vigorously for 16 h at 25° C. TLC showed the reaction was completed, staring material was consumed and the new spot was formed (TLC-10% MeOH:EtOAc; TLC charring—Phosphomolybdic acid). The hexane layer was separated by using separatory funnel. Again 300 mL of hexane was added to amine layer and stir for 4 h at rt. After that hexane layer was separated and combined with previous hexane layer, dried over sodium sulphate and evaporated to dryness under reduced pressure to get compound WLS-41b (48 g) as a crude colorless liquid. MS: m/z calcd for C₁₈H₄₀N₂ ([M+H]⁺), 285.53; found 285.38.

Preparation of compound WLS-41c: WLS-41b (48.0 g, 0.169 mol, 1.0 equiv) was taken in clean and dry 1 Lit two neck RBF under argon atmosphere. Then add 491 mL of THF to RBF. Cool the RB in ice bath (0° C. ). Add portion wise 1,1′-Carbonyldiimidazole (28.17 g, 0.174 mol, 1.03) to RM for period of 10 min. The reaction mixture was stir at 15° C. for 12 h. TLC showed the reaction was completed, staring material was consumed and the product was formed (TLC—10% MeOH:EtOAc; TLC charring—Phosphomolybdic acid). After completion of reaction, solvent was dried and purified on silica gel column chromatography (100-200 mesh). The product was eluted with 50% ethyl acetate: hexane. Fraction containing product was evaporated to get 37.1 g (71% yield) of WLS-41c as a white solid. ¹H NMR (400 MHz, CDCl₃): δ in ppm=4.33 (s, 1H), 3.40-3.43 (m, 4H), 3.17 (t, 2H, J=7.4 Hz), 1.50 (t, 2H, J=7.0 Hz), 1.25-1.30 (m, 28H), 0.88 (d, 3H, J=13.6 Hz). MS: m/z calcd for Ci₉H₃₈1\1₂0 ([M+H]⁺), 311.53; found 311.42.

Preparation of compound WLS-41d: WLS-41c (29.0 g, 0.093 mol, 1.0 equiv) was taken in clean and dry 1 Lit two neck RBF under argon atmosphere. Then add 471 mL of dry DMF to RBF containing SM. Cool the RB in ice bath (Temp. 0° C. ). Then, add portion wise 60% NaH (4.48 g, 0.112 mol, 1.20 equiv) to RM for period of 15 min. at 0° C. and stir 30 min at same temp. Then add dropwise methyl iodide (17.4 mL, 0.281 mol, 3.0 equiv) to the reaction mixture at 0° C. for duration of 15 min. Then allow the RM to rt and stir for 3 h. TLC showed the reaction was completed, staring material was consumed and the new spot was formed (TLC—EtOAc; TLC charring—Phosphomolybdic acid). After completion of reaction, reaction mixture was cool to 0° C. in ice bath and quenched with ice cold water (1 Lit). Then extracted with ethyl acetate (3×1000 mL). The organic layer was dried over sodium sulfate, filtered and concentrated to dryness. The crude product was purified by silica gel column chromatography (100-200 mesh). The product was eluted with 25%-35% ethyl acetate:hexane. The fraction containing product was evaporated to get 29.0 g (96% yield) of WLS-41d as a white colour solid. ¹H NMR (500 MHz, CDCl₃): δ in ppm=3.27 (s, 4H), 3.16 (t, 2H, J=7.6 Hz), 2.78 (s, 3H), 1.48 (t, 2H, J=7.2 Hz), 1.29 (s, 7H), 1.25 (s, 22H), 0.88 (t, 3H, J=6.9 Hz). MS: m/z calcd for C₂₀H₄₀N₂O ([M+H]⁺), 325.55; found 325.41.

Preparation of compound WLS-41e: WLS-41d (30.0 g, 0.092 mol, 1.0 equiv) was taken in clean and dry 1 Lit two neck RBF under argon atmosphere. Then add 249 mL of dry toluene to RBF containing SM under argon atmosphere. After that add dropwise oxalyl chloride (118.9 mL, 1.386 mol, 15.0) using addition funnel for a period of 30 min at rt. Then reaction mixture was heated to 65° C. for 72 hrs. After completion of reaction (TLC—ethyl acetate; TLC charring—Phosphomolybdic acid) solvent was evaporated to dryness to get crude compound. The crude compound was washed with cold ethyl acetate (2×100 mL) and dried to get 33.0 g of crude WLS-41e as brown colour solid. MS: m/z calcd for C₂₀H₄₀Cl₂N₂O ([M−Cl]⁺), 344.00; found 343.30.

Preparation of compound WLS-41f: WLS-41e (20.0 g, 0.053 mol, 1.0 equiv) was taken in clean and dry 500 mL single neck RBF and dissolved in 115 mL DCM under argon atmosphere. Then added aq solution of KPF₆ (9.70 g, 0.053 mol, 1.0 equiv,in 65 mL of water). Stir the reaction mixture at rt for 1 h. After completion of reaction (TLC—5% MeOH:DCM; TLC charring—Phosphomolybdic acid), the reaction mixture was poured into ice water, and extracted with DCM (2×400 mL). The combined organic layer washed with water (400 mL) and dried over sodium sulphate, filtered and evaporated to dryness. Then, residue was dissolved in DCM (70 mL) and product was precipitate by dropwise addition of diethyl ether (500 mL) under stirring. The solvent was decant and solid was dried under high vacuum to get 18.0 g (70% yield) of WLS-41f as a white solid. MS: m/z calcd for C₂₀H₄₀ClF₆N₂P ([M—PF₆]⁺), 344.00; found 343.34.

Preparation of compound WLS-41: WLS-41f (18.0 g, 0.037 mol, 1.0 equiv) was taken in clean and dry 500 mL single neck RBF and dissolved in 90 mL of Dry MeCN under argon atmosphere. Then, added sodium azide (3.58 g, 0.055 mol, 1.5 equiv) to the RM and stir at rt for 2.5 h. After completion of reaction (TLC—ethyl acetate; TLC charring—ninhydrin), reaction mixture was filtered through a pad of celite and washed with MeCN (20 mL). The organic layer was evaporated to dryness. The crude compound was dissolve in MeCN (70 mL) and precipitate by adding dropwise diethylether (500 mL). Solvent was decanted and solid was dried under high vacuum to get 14.1 g (77% yield) of WLS-41 as a white solid. ¹H NMR (400 MHz, CDCl₃): δ in ppm=3.94-4.00 (m, 2H), 3.85-3.90 (m, 2H), 3.41 (t, 2H, J=7.6 Hz), 3.21 (s, 3H), 1.62 (t, 2H, J=7.1 Hz), 1.26 (s, 27H), 0.88 (t, 3H, J=6.8 Hz). ¹⁹F NMR (400 MHz, CDCl₃): 6 in ppm=-73.35 and -75.24. MS: m/z calcd for C₂₀H₄₀F₆N₅P ([M−PF₆]⁺), 350.57; found 350.40. IR (KBr pellet): N₃ (2179 cm⁻¹).

In some embodiments, commercially available azides were utilized, e.g.,:

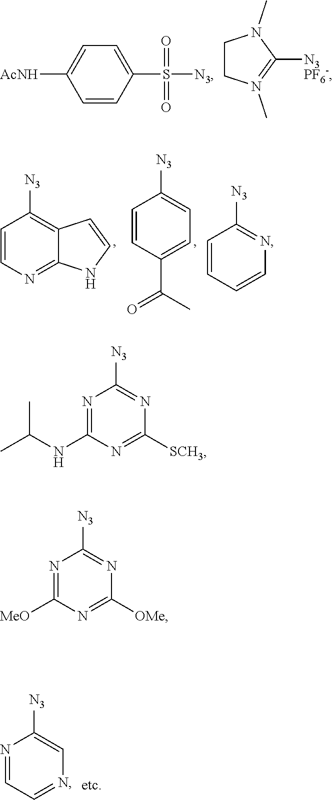

### Example 22. Synthesis of N103-009

### Preparation of 2-chloro-4,5-dimethyl-1,3,2-oxathiaphospholane

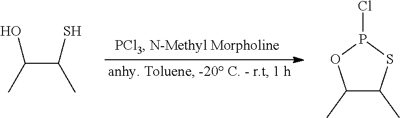

50 g of mercapto-2-butanol and 114 mL of N-Methyl morpholine were added to 600 mL toluene. In a separate round bottomed flask 45 mL of PCl₃ was added to 400 mL toluene and cooled to 0° C. The mercapto-2-butanol solution was cannulated into the PCl₃ solution over 20 minutes keeping the temperature below 20° C. The reaction mixture was warmed to room temperature for an hour and was vacuum filtered and washed with toluene. The material was concentrated under reduced pressure to afford 2-chloro-4,5-dimethyl-1,3,2-oxathiaphospholane a pale-yellow oil (quantitative yield) and used for further step. ³¹P NMR (162 MHz, CDCl₃) δ 205.72, 205.40.

### Preparation of (2S)-2-((bis(4-methoxyphenyl)(phenyl)methoxy)methyl)-4-(4,5-dimethyl-1,3,2-oxathiaphospholan-2-yl) morpholine (N103-009)

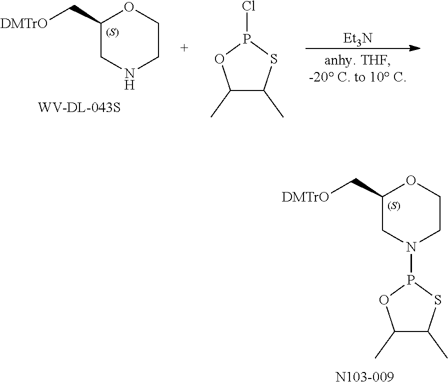

(S)-2-((bis(4-methoxyphenyl)(phenyl)methoxy)methyl)morpholine (WV-DL-0435, 16.4 g, 39 mmole) was dried two times by co-evaporation with 50 mL of anhydrous toluene at 40 oC and kept at high vacuum for overnight. Then the dried WV-DL-043S was dissolved in dry THF (100 mL), in 500 mL three neck flasks under argon, followed by the addition of triethylamine (20.2 g, 28 mL, 200 mmole) and the was cooled to -20 oC. To this cooled reaction mixture was added the solution of the crude 2-chloro-4,5-dimethyl-1,3,2-oxathiaphospholane (60 mmole, 10.3 g, 1.5 eq,)dissolved in THF 40 mL was added through syringe dropwise —15min (maintain the internal temperature −20° C. then gradually warmed to 10C. After 30min at 10 oC, TLC and LCMS analysis indicated the complete conversion of SM to product (total reaction time 2 h). The reaction was filtered through Airfree, Schlenk filter tube, washed with dry THF (50 mL) and evaporated under rotary evaporation at 30° C. afforded the gummy solid was dried under high vacuum for overnight. The dried crude product was purified by Combi-Flash Rf (Teledyne ISCO) using 220 silica column (which was pre-deactivated with 3 column volume of ethyl acetate with 5% TEA) with ethyl acetate/Hexane mixture as a solvent. After column fractions were analyzed by TLC and LCMS pooled together and evaporated in a reevaporated at 30 oC and was dried under high vacuum afforded pale yellow solid (2 S)-2-((bis (4-methoxyphenyl) (phenyl)methoxy)methyl)-4-(4,5-dimethyl-1,3 ,2-oxathiapho spholan-2-yl) morpholine (N103-009). Yield: 14 g (65%). Chemical Formula: 30H36NO5PS; Calculated Molecular Weight: 553.65; Observed Mass in LCMS m/z: 554.58 (M+H). ¹H NMR (400 MHz, Chloroform-d) δ 7.45 (dddd, J=7.0, 6.0, 3.0, 1.9 Hz, 3H), 7.38-7.17 (m, 10H), 6.88-6.79 (m, 5H), 3.83 (s, 1H), 3.79 (s, 8H), 3.62-3.39 (m, 4H), 3.31-3.16 (m, 2H), 3.16-2.95 (m, 3H), 2.05 (s, 1H), 1.49-1.17 (m, 9H). ³¹P NMR (162 MHz, CDCl₃) δ 156.44, 156.32, 151.34, 151.20, 141.44, 140.46, 135.99, 135.94.

### Example 23. Synthesis of N103-010

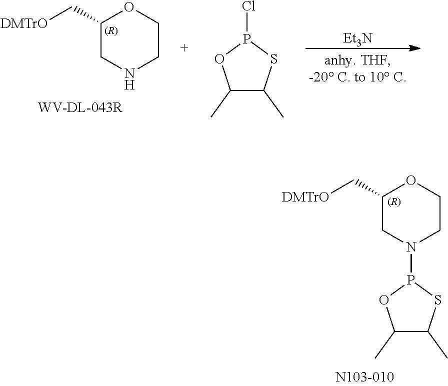

(R)-2-((bis(4-methoxyphenyl)(phenyl)methoxy)methyl)morpholine (WV-DL-043R, 8.0 g, 19 mmole) was dried two times by co-evaporation with 35 mL of anhydrous toluene at 40 oC and kept at high vacuum for overnight. Then the dried WV-DL-043R was dissolved in dry THF (80 mL), in 500 mL three neck flasks under argon, followed by the addition of triethylamine (9.6 g, 13.5 mL, 95 mmole) and the was cooled to -20 oC. To this cooled reaction mixture was added the solution of the crude 2-chloro-4,5-dimethyl-1,3,2-oxathiaphospholane (29 mmole, 5.0 g, 1.5 eq,)dissolved in THF 30 mL was added through syringe dropwise ˜10min (maintain the internal temperature −20° C. then gradually warmed to 10C. After 30min at 10 oC, TLC and LCMS analysis indicated the complete conversion of SM to product (total reaction time 2 h). The reaction was filtered through Airfree, Schlenk filter tube, washed with dry THF (40 mL) and evaporated under rotary evaporation at 30° C. afforded the gummy solid was dried under high vacuum for overnight. The dried crude product was purified by Combi-Flash Rf (Teledyne ISCO) using 120 silica column (which was pre-deactivated with 3 column volume of ethyl acetate with 5% TEA) with ethyl acetate/hexane mixture as a solvent. After column fractions were analyzed by TLC and LCMS pooled together and evaporated in a reevaporated at 30 oC and was dried under high vacuum afforded pale yellow solid (2R)-2-((bis(4-methoxyphenyl) (phenyl)methoxy)methyl)-4-(4,5-dimethyl-1,3,2-oxathiaphospholan-2-yl) morpholine (N103-010). Yield: 6.5 g (62%). Chemical Formula: C30H36NO5PS; Calculated Molecular Weight: 553.65; Observed Mass in LCMS m/z: 554.59 (M+H). 1H NMR (400 MHz, Chloroform-d) δ 7.45 (dddd, J=7.0, 6.0, 3.0, 1.9 Hz, 3H), 7.38-7.17. (m, 10H), 6.88-6.79 (m, 5H), 4.13 (q, J=7.2 Hz, 1H), 3.83 (s, 1H), 3.79 (s, 8H), 3.62-3.39. (m, 4H), 3.31-3.16 (m, 2H), 3.16-2.95 (m, 3H), 2.87-2.57 (m, 1H), 2.05 (s, 1H), 1.49-1.17 (m, 9H). ³¹P NMR (202 MHz, CDCl₃) δ 156.20, 156.08, 151.10, 150.97, 141.15, 140.18, 135.70, 135.65.

Certain useful amidites:

For linker L010n001:

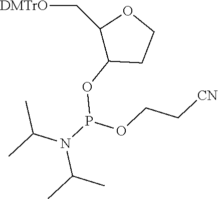

For linker L009n001:

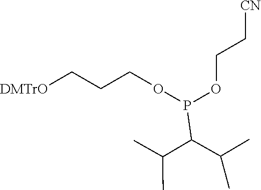

For linker L023:

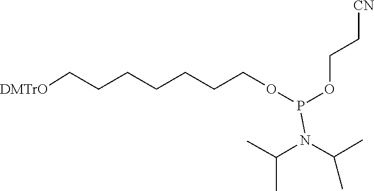

### Example 24. Synthesis of PS—PN and PS—PS Dimers

General experimental procedure (A) for chloro reagent (2)

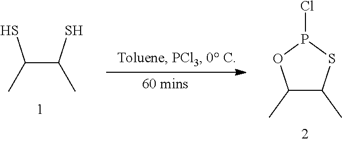

Dithiol (360 mmol) was dissolved in toluene (720 mL) under argon (3000 mL single neck flask) then 4-methylmorpholine (35.4 mL, 792 mmol) was added. This mixture was added dropwise via cannula over 30 min to an ice-cold solution of phosphorus trichloride (720 mL, 396 mmol) in toluene (720 mL) under argon atmosphere. After warming to room temperature for 1 h, the mixture was filtered carefully under vacuum/argon. The resulting filtrate was concentrated by rotary evaporation (flushing with Ar) then dried under high vacuum for 2 h. The resulting crude compound was isolated as thick oil, which was dissolved in THF to obtain a 1 M stock solution and this solution was used in the next step without further purification. Compound 2: Synthesized from compound 1, by following the general procedure A. ³¹P NMR (243 MHz, THF-CDCl₃, 1:2) δ 168.77, 161.4

General experimental procedure (B) for monomers (5 and 6)

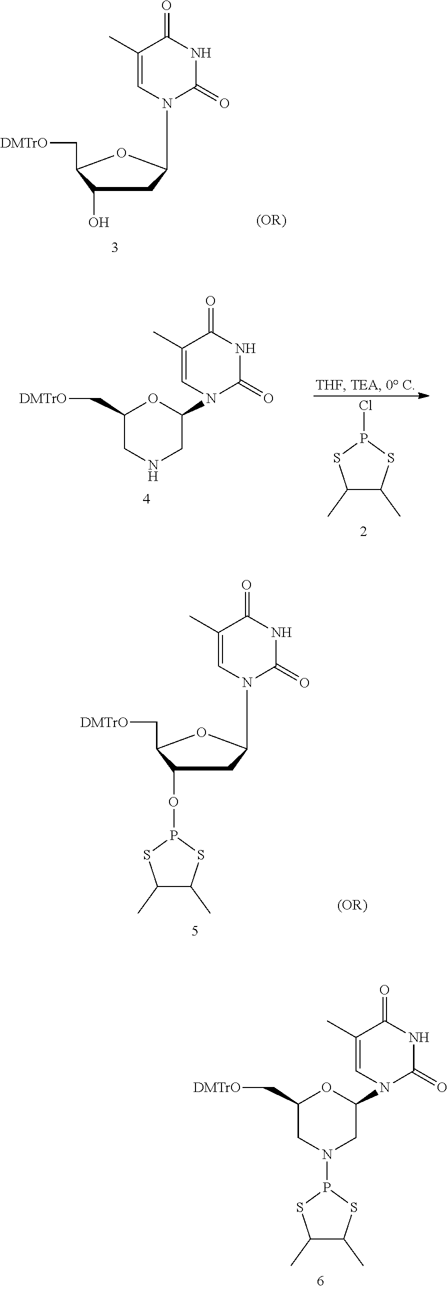

The 5′-ODMTr protected nucleoside Compound 3 or Compound 4 (6.9 mmol) was dried in a three neck 250 mL round bottom flask by co-evaporating with anhydrous toluene (50 mL) followed by under high vacuum for 18 h. The dried nucleoside was dissolved in dry THF (35 mL) under argon atmosphere. Then, triethylamine (24.4 mmol, 3.5 equiv.) was added into the reaction mixture, then cooled to ˜−10° C. A THF solution of the crude chloro reagent (1 M solution, 2.5 equiv., 17.4 mmol) was added to the above mixture through cannula over ˜5 min, then, gradually warmed to room temperature over about 1 h. LCMS showed that the starting material was consumed. The reaction mixture was filtered carefully under vacuum/argon and the resulting filtrate was concentrated under reduced pressure to give a yellow foam which was further dried under high vacuum overnight. Crude mixture was purified by silica gel column [Column was pre-deactivated using acetonitrile then ethyl acetate (5% TEA) and then equilibrated using ethyl acetate-hexanes] chromatography using ethyl acetate and hexane as eluents.

Compound 5, Stereorandom (Rp/Sp) monomer: Yield 86%. Reaction was carried out using nucleoside 3 and chloro reagent 2 by following the general procedure B. ³¹P NMR (243 MHz, CDCl₃) δ 171.62, 155.50, 146.84, 146.17; MS (ES) m/z calculated for C₃₅H₃₉N₂O₇PS₂ [M+K]⁺733.16, Observed: 733.40 [M+K]⁺.

Compound 6, Stereorandom (Rp/Sp) monomer: Yield 73%. Reaction was carried out using nucleoside 4 and chloro reagent 2 by following the general procedure B. ³¹P NMR (243 MHz, CDCl₃) δ 121.87, 106.20, 93.58, 92.99; MS (ES) m/z calculated for C₃₅H₄₀N₃O₆PS2 [M+K]⁺773.28, Observed: 773.70 [M+K]⁺.

General experimental procedure (C) for PS—PN dimers (7 and 8):

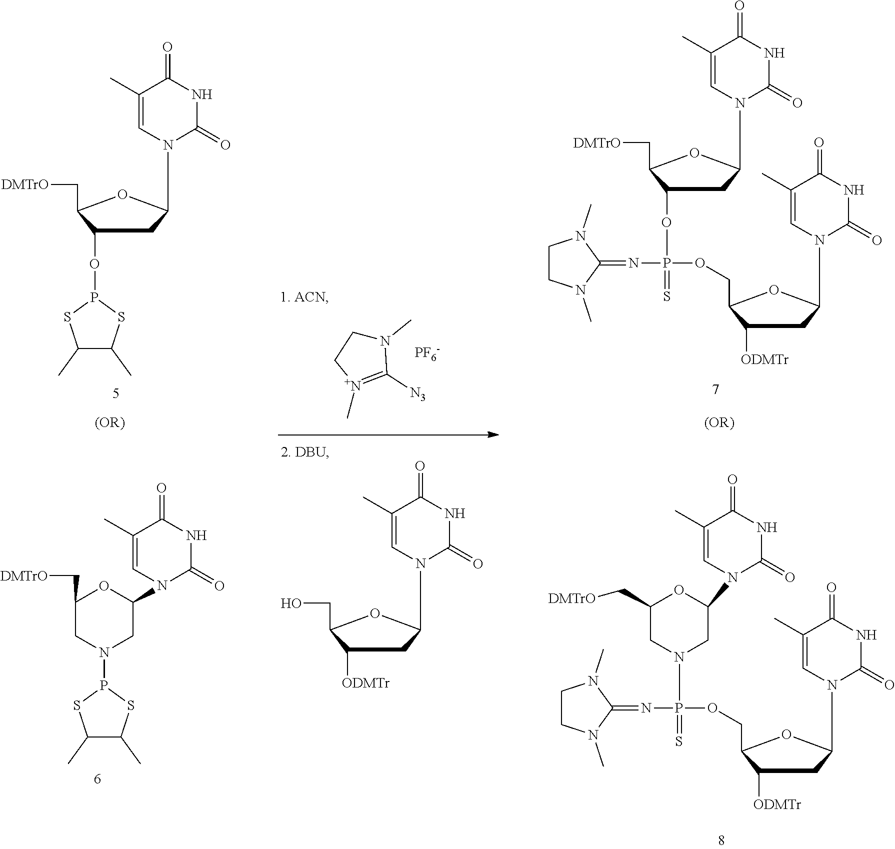

To a stirred solution of monomer 5 or 6 (0.10 mmol, 2 equiv., pre-dried by co-evaporation with dry acetonitrile and kept it under vacuum for minimum 12 h) in dry acetonitrile (0.5 mL) was added a solution of 2-azido-1,3-dimethylimidazolinium hexafluorophosphate (0.11 mmol, 2.25 equiv.) in acetonitrile (0.2 mL) under argon atmosphere at room temperature. Resulting reaction mixture was stirred for 10 mins then DMTr protected alcohol (0.05 mmol, pre-dried by co-evaporation with dry acetonitrile and kept it under vacuum for minimum 12 h) in dry acetonitrile (0.25 mL) and 1,8-Diazabicyclo [5.4.0] undec-7-ene (0.23 mmol, 5 equ, 0.23 ml of 1 M solution in dry acetonitrile) are added. The reaction was monitored and analyzed by LCMS. Approximate reaction completion time 10-20 mins.

Compound 7: Reaction was carried out using 5 by following the general procedure C. MS (ES) m/z calculated for C₆₇H₇₂N₇O₁₄PS [M+K]⁺1300.42, Observed: 1300.70 [M+K]⁺.

Compound 8: Reaction was carried out using 6 by following the general procedure C. MS (ES) m/z calculated for C₆₇H₇₃N₈O₁₃PS [M+K]⁺1299.44, Observed: 1299.65 [M+K]⁺.

General experimental procedure (D) for PS—PS dimers (9 and 10):

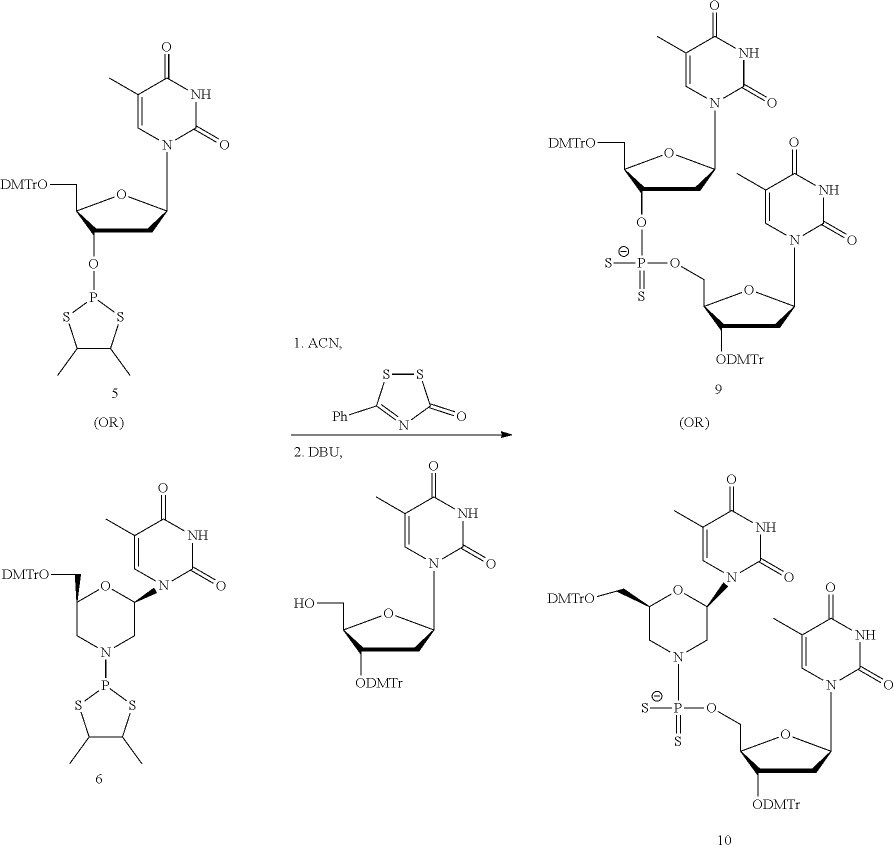

To a stirred solution of monomer 5 or 6 (0.10 mmol, 2 equiv., pre-dried by co-evaporation with dry acetonitrile and kept it under vacuum for minimum 12 h) in dry acetonitrile (0.5 mL) was added a solution of 5-phenyl-3H-1,2,4-dithiazol-3-one (0.12 mmol, 2.5 equiv., 0.2 M) in acetonitrile under argon atmosphere at room temperature. Resulting reaction mixture was stirred for 10 mins then DMTr protected alcohol (0.05 mmol, 1 equ, pre-dried by co-evaporation with dry acetonitrile and kept it under vacuum for minimum 12 h) in dry acetonitrile (0.2 mL) and 1,8-Diazabicyclo [5.4.0] undec-7-ene (0.23 mmol, 5 equ, 1 M solution in dry acetonitrile) are added. Once the reaction was completed (monitored by LCMS) then the reaction mixture was analyzed by LCMS.

Compound 9: Reaction was carried out using monomer 5 by following the general procedure D. Reaction completion time about 30 mins. MS (ES) m/z calculated for C₆₂H₆₂N₄O₁₄PS₂ [M]⁻1181.34, Observed: 1181.66 [M]⁻.

Compound 10: Reaction was carried out using monomer 6 by following the general procedure D. Reaction completion time about 20 h. MS (ES) m/z calculated for C₆₂H₆₃N₅O₁₃PS₂ [M]⁻1180.36, Observed: 1180.71 [M]⁻.

Additional useful compounds were prepared utilizing various technologies in accordance with the present disclosure. Certain compounds are described below as examples.

WV-NU-161A and WV-NU-161A-CNE (e.g., useful for WV-39291).

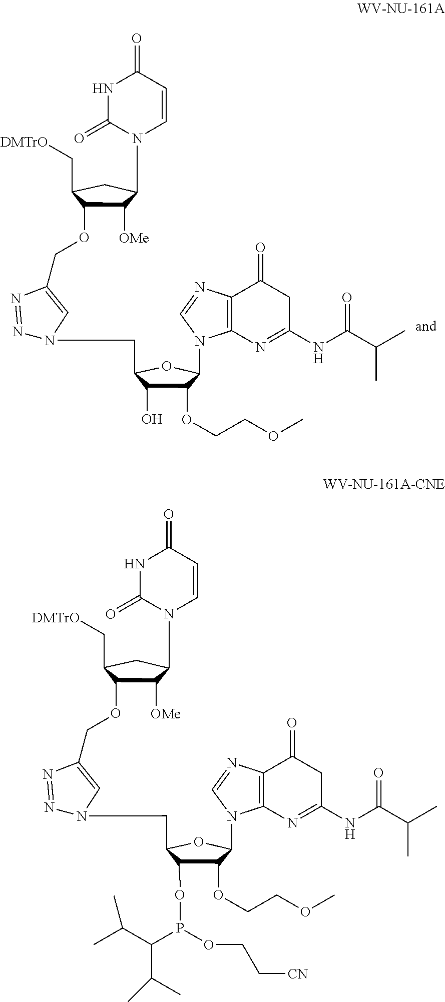

General synthetic route:

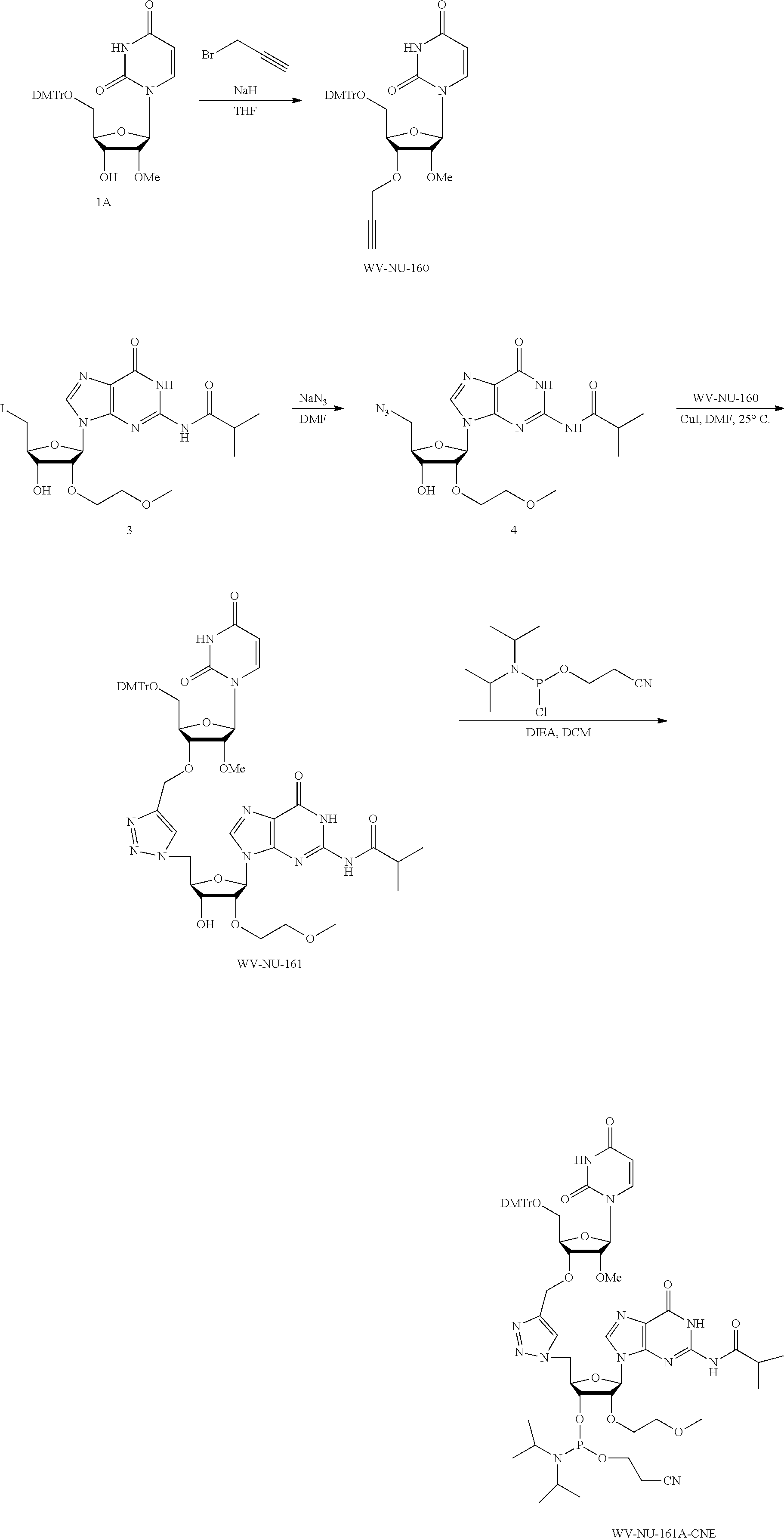

Step 1A. Preparation of WV-NU-160.

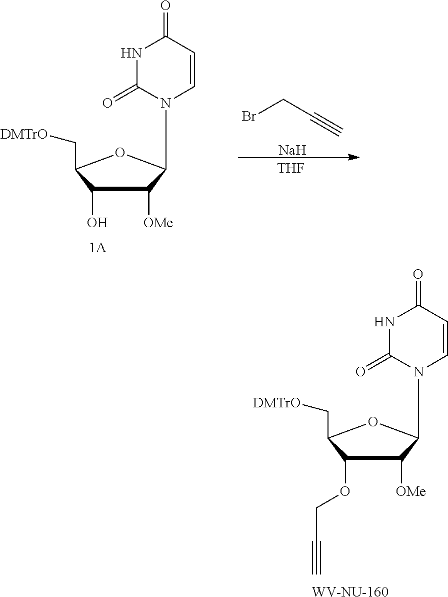

To a solution of compound 1A (30 g, 53.51 mmol, 1 eq.) in THF (300 mL) was added dropwise NaH (5.35 g, 133.79 mmol, 60% purity, 2.5 eq.) at 0° C. with N₂ for 3 times. After addition, the mixture was stirred at this temperature for 0.5 hr, and then 3-bromoprop-1-yne (14.32 g, 120.41 mmol, 10.38 mL, 2.25 eq.) was added at 20° C. The resulting mixture was stirred at 20° C. for 12 hrs under N₂ atmosphere. The reaction mixture was added MeOH 100 mL and concentrated under reduced pressure to give a residue. The residue was purified by column chromatography (SiO₂, Petroleum ether/Ethyl acetate=100/1 to 1/0) to get the compound WV-NU-160 (22.1 g, 36.92 mmol, 68.98% yield) was obtained as yellow solid. LCMS (M−H⁺):597.2. TLC: Petroleum ether/Ethyl acetate=1:2, Rf=0.20.

Step 2A. Preparation of compound 2.

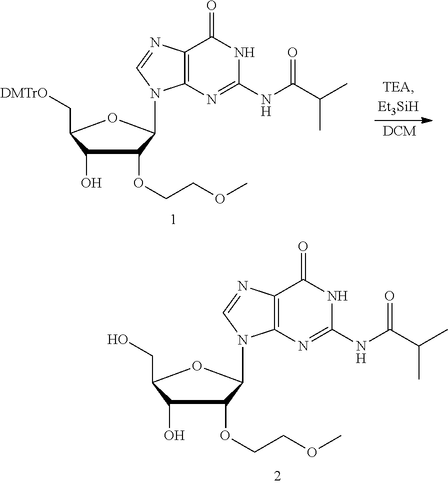

To a solution of compound 1 (37 g, 51.84 mmol, 1 eq.) in DCM (1200 mL) was added TFA (11.82 g, 103.67 mmol, 7.68 mL, 2 eq.), the mixture was stirred at 15° C. for 12 hr. TLC (Ethyl acetate: Methanol=5: 1, Rf=0.15) showed the reactant 1 was consumed and the desired substance was found. The mixture was concentrated to get the crude at 20° C. The residue was purified by MPLC (Ethyl acetate: Methanol=0:1, 3:1) to get compound 2 (21 g, 51.04 mmol, 98.47% yield) as a white solid. LCMS (M−H+): 410.1. TLC (Ethyl acetate : Methanol=5:1, Rf=0.15).

Step 3A. Preparation of compound 3.

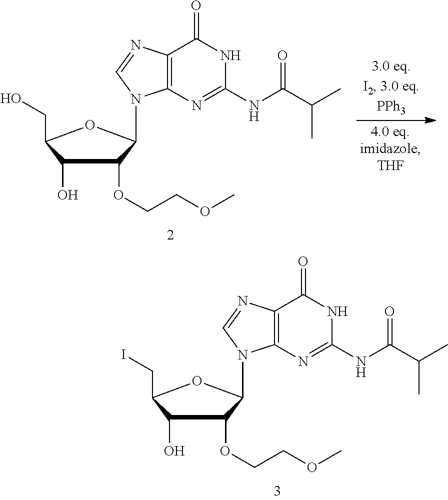

To a solution of compound 2 (21 g, 51.04 mmol, 1 eq.), PPh₃ (40.16 g, 153.13 mmol, 3 eq.), IMIDAZOLE (13.90 g, 204.18 mmol, 4 eq.) in THF (200 mL) was added 12 (38.87 g, 153.13 mmol, 30.85 mL, 3 eq.) in THF (200 mL), the mixture was stirred at 25° C. for 5 hr. TLC (Ethyl acetate: Methanol=5:1). The mixture was concentrated to get the crude and diluted with DCM (50 mL), the residue was purified by silica gel chromatography (Ethyl acetate: Methanol=0:1, 5:1) to get compound 3 (22 g, 42.20 mmol, 82.68% yield) as a yellow solid. LCMS (M+H+): 522.1. TLC (Petroleum ether: Ethyl acetate=0:1), Rf=0.38.

Step 4A. Preparation of compound 4.

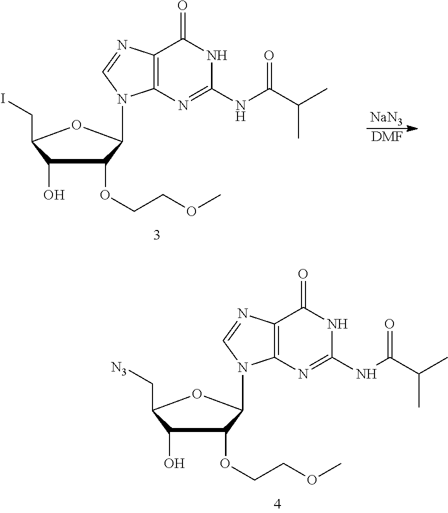

To a solution of compound 3 (24 g, 46.04 mmol, 1 eq.) in DMF (200 mL) was added NaN₃ (9.22 g, 141.82 mmol, 3.08 eq.), the mixture was stirred at 80° C. for 3 hrs. The reaction mixture was added water 200.0 mL and extracted by EtOAc 200 mL*5. The combined organic layers were dried over Na₂SO₄, filtered and concentrated under reduced pressure to compound 4 (20 g, crude) as a yellow oil. LCMS: (M+H+): 437.2.

Step 5A. Preparation of WV-NU-161A

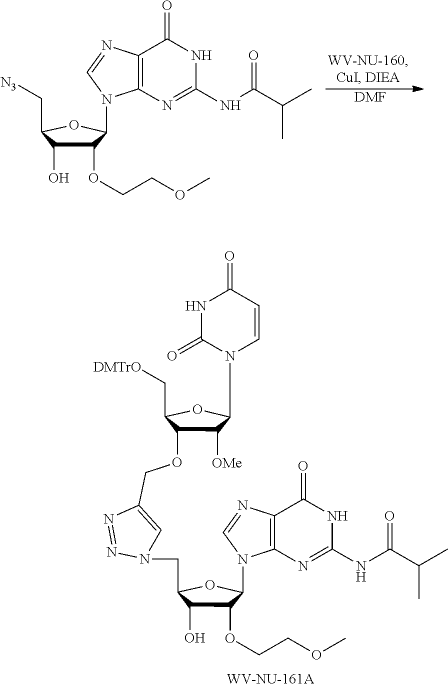

To a solution of compound 4 (16 g, 36.66 mmol, 0.8 eq.) and WV-NU-160 (27.43 g, 45.83 mmol, 1 eq.), DIEA (11.85 g, 91.65 mmol, 15.96 mL, 2 eq.) in DMF (160 mL) was added CuI (1.75 g, 9.17 mmol, 0.2 eq.) and the mixture was stirred at 15° C. for 12 hr. LCMS showed the compound 4 was consumed and the desired substance was found. The reaction mixture was diluted with water (100 mL), extracted with DCM (300 mL*5) and concentrated under reduced pressure to give a residue. The residue was purified by silica gel chromatography (Ethyl acetate : Methanol=1/0, 10/1, 5:1, 5%TEA) to get the crude 30 g and then the mixture was washed with Ethyl acetate : Methanol=10:1 (50 mL) filtered and the cake was dried to get the WV-NU161A (21 g, 20.29 mmol, 44.27% yield) as a yellow solid. Two batches: Batchl: (9.2 g). ¹HNMR (400 MHz, DMSO-d6) δ=12.09 (br s, 1H), 11.70-11.29 (m, 2H), 8.17 (s, 1H), 8.01 (s, 1H), 7.79 (d, J=8.1 Hz, 1H), 7.36-7.27 (m, 4H), 7.25-7.13 (m, 5H), 6.89 (d, J=8.9 Hz, 4H), 5.91 (d, J=5.5 Hz, 1H), 5.78 (d, J=3.1 Hz, 1H), 5.44 (d, J=4.9 Hz, 1H), 5.22 (d, J=8.0 Hz, 1H), 4.77-4.63 (m, 2H), 4.60 (d, J=2.4 Hz, 2H), 4.46-4.37 (m, 2H), 4.36-4.32 (m, 1H), 4.30-4.24 (m, 1H), 4.10-4.06 (m, 1H), 3.73 (s, 6H), 3.72-3.67 (m, 1H), 3.60-3.54 (m, 1H), 3.40 (t, J=4.7 Hz, 2H), 3.36 (s, 3H), 3.30-3.17 (m, 3H), 3.15 (s, 3H), 2.75 (quin, J=6.8 Hz, 1H), 1.11 (dd, J=1.3, 6.8 Hz, 6H). LCMS purity: 98.55%, (M−H+):1033.3. Batch 2 (11.8 g): ¹HNMR (400 MHz, DMSO-d6) δ=12.11 (br s, 1H), 11.61 (s, 1H), 11.43 (s, 1H), 8.20 (s, 1H), 8.03 (s, 1H), 7.81 (d, J=8.1 Hz, 1H), 7.40-7.29 (m, 5H), 7.28-7.14 (m, 7H), 6.91 (d, J=8.9 Hz, 5H), 5.93 (d, J=5.5 Hz, 1H), 5.79 (d, J=3.0 Hz, 1H), 5.47 (d, J=4.9 Hz, 1H), 5.24 (d, J=8.1 Hz, 1H), 4.81-4.64 (m, 2H), 4.64-4.57 (m, 2H), 4.46-4.39 (m, 2H), 4.39-4.22 (m, 3H), 4.13-4.07 (m, 2H), 3.75 (s, 7H), 3.64-3.55 (m, 1H), 3.41 (t, J=4.6 Hz, 3H), 3.17 (s, 3H), 1.13 (dd, J=1.3, 6.8 Hz, 7H). LCMS purity: 94.13%, (M−H+):1033.3. TLC Ethyl acetate : Methanol=5:1, Rf=0.25.

Step 6A. Preparation of WV-NU-161A-CNE.

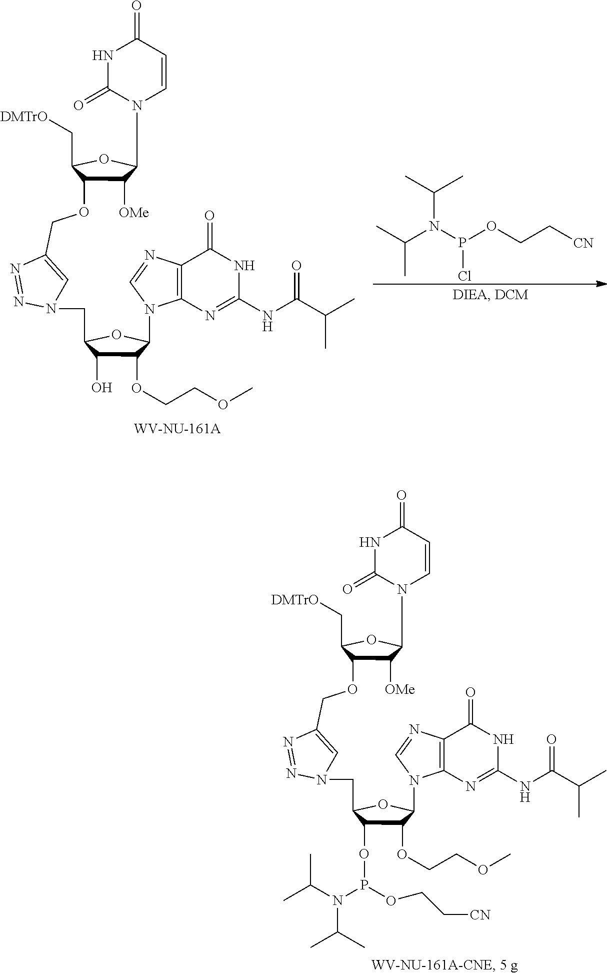

WV-NU161A (7 g, 6.76 mmol, 1 eq.) was dried by azeotropic distillation on a rotary evaporator with toluene (30 mL*3). To a solution of WV-NU161A (7 g, 6.76 mmol, 1 eq.) in DCM (80 mL) was added DIEA (1.75 g, 13.53 mmol, 2.36 mL, 2 eq.) then 3-[chloro(diisopropylamino)phosphanyl]oxypropanenitrile (2.40 g, 10.14 mmol, 1.5 eq.). The mixture was stirred at 15° C. for 2 hr. TLC (Ethyl acetate: Methanol=5:1, Rf=0.43) indicated Reactant 1 was consumed and one new spot formed. The reaction mixture was quenched by addition sat. NaHCO₃ aq. (20 mL) at 0° C., and extracted with DCM (30 mL*3), dried over Na₂SO₄, filtered and concentrated under reduced pressure to give a residue. The residue was purified by column chromatography (SiO₂, Petroleum ether/Ethyl acetate=1/0 to 0/1, then EtOAc/ACN=1/0 to 1/1, 5% TEA) to get WV-NU161A-CNE (3.4 g, 2.75 mmol, 40.70% yield) as a white solid. ¹HNMR (400 MHz, DMSO-d6) δ=11.70-11.27 (m, 2H), 8.31-8.18 (m, 1H), 8.08-7.98 (m, 1H), 7.81 (dd, J=2.3, 8.1 Hz, 1H), 7.40-7.12 (m, 9H), 6.90 (br d, J=8.6 Hz, 4H), 6.01-5.83 (m, 1H), 5.79 (d, J=2.6 Hz, 1H), 5.24 (br d, J=8.0 Hz, 1H), 4.82-4.56 (m, 6H), 4.53-4.31 (m, 2H), 4.15-4.04 (m, 2H), 3.89-3.79 (m, 1H), 3.73 (s, 7H), 3.68-3.48 (m, 4H), 3.36 (br d, J=8.3 Hz, 11H), 3.19-3.08 (m, 3H), 2.86-2.68 (m, 3H), 1.16-1.05 (m, 14H). ³¹P NMR (162 MHz, DMSO-d6) δ=150.28 (s, 1P), 149.82 (s, 1P). TLC (Ethyl acetate: Methanol=5:1), Rf=0.43

WV-NU-163 & compound 11.

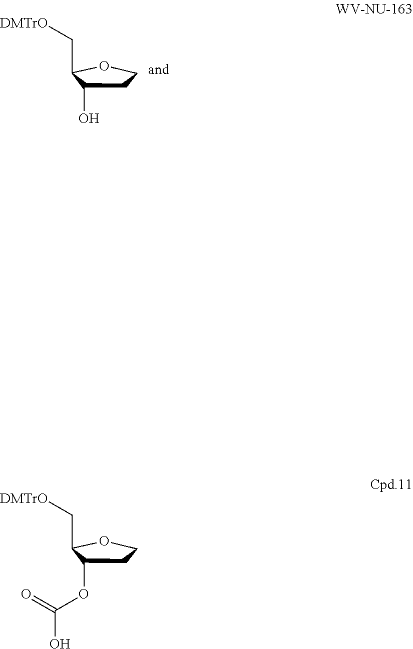

General synthetic route:

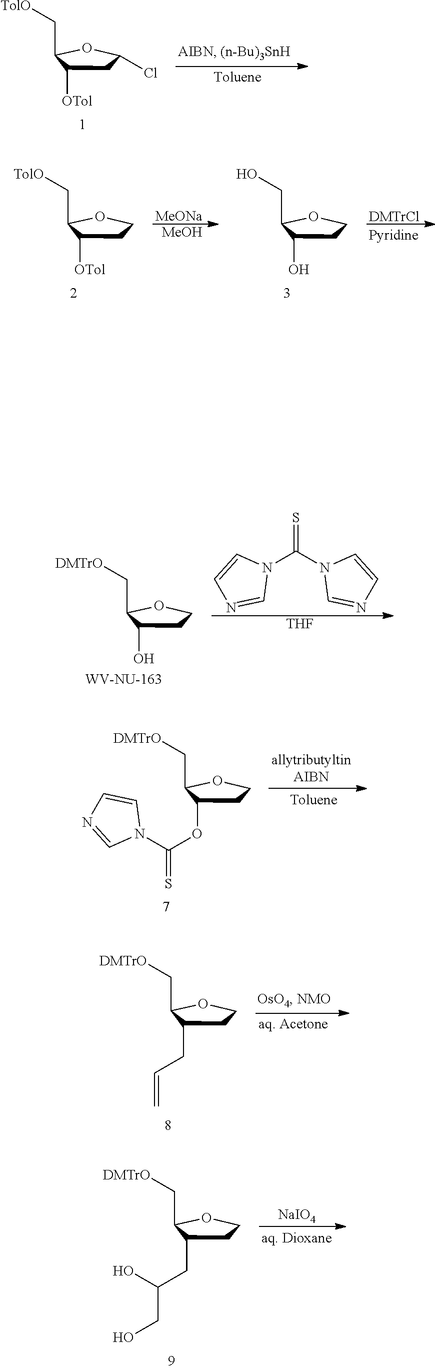

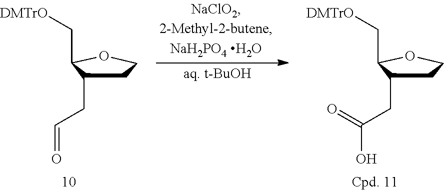

Step 1B Preparation of compound 2.

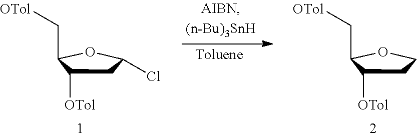

The compound 1 (125 g, 321.47 mmol, 1 eq.) was dissolved in dry toluene (1250 mL) and the AIBN (1.98 g, 12.06 mmol, 3.75e-2 eq.) and (n-Bu)₃SnH (93.57 g, 321.47 mmol, 85.06 mL, 1 eq.) were added. The solution was heated to 100° C. for 3 h. TLC (Petroleum ether: Ethyl acetate=5: 1, Rf=0.39) indicated compound 1 was consumed completely and one new spot formed. The two batches were combined for work up. The mixture was purified by silica gel chromatography (Petroleum ether/Ethyl acetate=100/1, 50/1, 30/1) to get compound 2 (200 g, 564.34 mmol, 87.78% yield) as a yellow oil. TLC: Petroleum ether: Ethyl acetate=5: 1, Rf=0.39.

Step 2B. Preparation of compound 3.

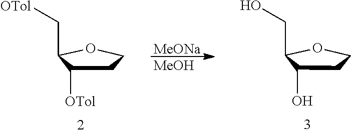

To a solution of compound 2 (200 g, 564.34 mmol, 1 eq.) in MeOH (2 L) was added NaOMe (91.46 g, 1.69 mol, 3 eq.), the mixture was stirred at 25° C. for 3 hr. TLC Plate 1(Petroleum ether: Ethyl acetate=5:1) showed the reactant 1 was consumed and TLC Plate 2 (Ethyl acetate: Methanol=10:1, Rf=0.21) showed a new spot was found. NH₄Cl (91.5 g) was added and the mixture was concentrated to get the compound 3 (66.6 g, crude) as a yellow oil. TLC: Ethyl acetate: Methanol=10:1, Rf=0.21.

Step 3B. Preparation of WV-NU-163.

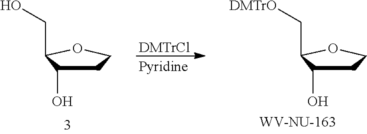

To a solution of compound 3 (66.6 g, 563.78 mmol, 1 eq.) in PYRIDINE (700 mL) was added DMTC1 (229.23 g, 676.54 mmol, 1.2 eq.). The mixture was stirred at 15° C. for 12 hr. TLC (Petroleum ether: Ethyl acetate=0:1, Rf=0.8) indicated compound 3 was consumed completely and two new spots formed. The reaction mixture was diluted with H₂O 1000 mL and extracted with EtOAc 3000 mL (1000 mL * 3). The combined organic layers were dried over Na₂SO₄, filtered and concentrated under reduced pressure to give a residue. The residue was purified by column chromatography (SiO₂, Petroleum ether/Ethyl acetate=10/1 to 0/1). Compound 4 (170 g, 398.87 mmol, 70.75% yield, 98.66% purity) was obtained as a yellow oil. ¹HNMR (400 MHz, CHLOROFORM-d) δ=7.44 (d, J=7.38 Hz, 2H), 7.19-7.36 (m, 8H), 6.83 (d, J=8.76 Hz, 4H), 4.30 (dq, J=6.49, 3.38 Hz, 1H), 3.99 (dd, J=8.25, 5.63 Hz, 2H), 3.86-3.91 (m, 1H), 3.80 (s, 6H), 3.25 (dd, J=9.57, 4.82 Hz, 1H), 3.09 (dd, J=9.57, 6.19 Hz, 1H), 2.10-2.21 (m, 1H), 1.85-1.94 (m, 1H), 1.77 (d, J=4.00 Hz, 1H). LCMS (M+H):419.1, LCMS purity: 98.66%.

Step 4B. Preparation of compound 7.

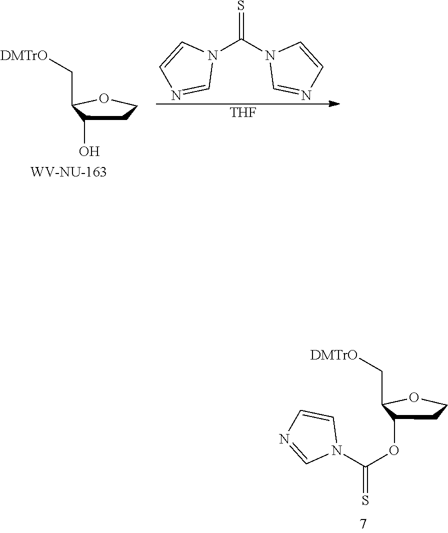

To a solution of compound WV-NU-163 (135 g, 321.05 mmol, 1 eq.) in THF (1500 mL) was added di(imidazol-1-yl)methanethione (85.82, 481.57 mmol, 1.5 eq.) under N₂. The mixture was stirred and reflux at 75° C. for 16 hr. TLC (Petroleum ether: Ethyl acetate=1:1, Rf=0.4) indicated WV-NU-163 was consumed completely one new spot formed. The reaction was clean according to TLC. showed WV-NU-163 was consumed completely and one main peak with desired m/z. The reaction mixture was concentrated under reduced pressure to remove solvent. The residue was purified by column chromatography (SiO₂, Petroleum ether/Ethyl acetate=10/1 to 1/1). Compound 7 (150 g, crude) was obtained as yellow oil. LCMS (M+H⁻): 531.4; purity: 62.35%. TLC: Petroleum ether: Ethyl acetate=1:1, Rf=0.4.

Step 5B. Preparation of compound 8.

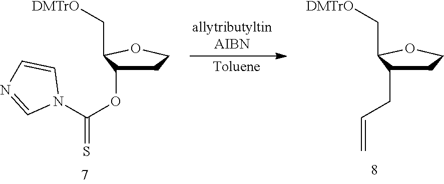

To a solution of compound 7 (122 g, 144.85 mmol, 63% purity, 1 eq.) in toluene (1200 mL) was added AIBN (16.65 g, 101.39 mmol, 0.7 eq.) and allyltributyltin (239.81 g, 724.23 mmol, 222.04 mL, 5 eq.). The mixture was stirred at 110° C. for 15 hr. TLC (Petroleum ether : Ethyl acetate=4/1.Rf=0.8) indicated compound 7 was consumed completely and three new spots formed. The reaction mixture of two batches were combined and quenched by addition H₂O (1000 mL), and then extracted with EtOAc (1000 mL * 3). The combined organic layers were concentrated under reduced pressure to give a residue and the solvent was quenched by sat.KF (aq.) 2000 mL. The crude of combined purified by column chromatography (SiO₂, Petroleum ether/Ethyl acetate=50/1 to 20/1). Compound 8 (60 g, 134.96 mmol, 93.18% yield) was obtained as a yellow oil. ¹HNMR (400 MHz, CHLOROFORM-d) δ=7.48 (br d, J=7.23 Hz, 2H), 7.18-7.41 (m, 7H), 6.83 (br d, J=8.99 Hz, 4H), 5.66-5.77 (m, 1H), 4.94-5.06 (m, 1H), 3.84-3.92 (m, 2H), 3.80 (s, 6H), 3.74 (q, J=5.26 Hz, 1H), 3.10-3.16 (m, 2H), 2.16-2.26 (m, 1H), 1.61-1.70 (m, 1H), 1.44 (s, 1H), 0.83-1.00 (m, 5H).

Step 6B. Preparation of compound 9.

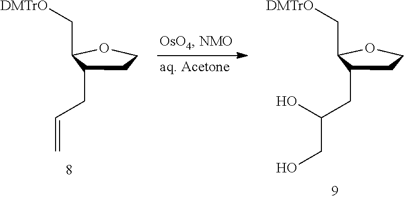

To a solution of compound 8 (59 g, 132.72 mmol, 1 eq.) in the mixed solvent of ACETONE (600 mL) and H₂O (60 mL) was added NMO (17.10 g, 145.99 mmol, 15.41 mL, 1.1 eq.) and tetraoxoosmium (5.06 g, 19.91 mmol, 1.03 mL, 0.15 eq.). The mixture was stirred at 15° C. for 2.5 hr. The reaction mixture was quenched by addition Na₂SO_{3 1000} mL, and then extracted with Ethyl acetate 1200 mL (400 mL *3). The combined organic layers were dried over Na₂SO₄, filtered and concentrated under reduced pressure to give a residue. Compound 9 (51 g, crude) was obtained as black brown oil. ¹HNMR (400 MHz, CHLOROFORM-d) δ=7.44-7.51 (m, 2H), 7.24-7.39 (m, 8H), 6.83 (dd, J=8.88, 2.96 Hz, 4H), 3.82-3.92 (m, 2H), 3.79 (s, 6H), 3.72 (br dd, J=9.21, 4.60 Hz, 2H), 3.39 (br s, 1H), 3.11-3.25 (m, 2H), 2.18-2.48 (m, 2H), 1.29-1.41 (m, 3H), 0.89-0.96 (m, 3H).

Step 7B. Preparation of compound 10.

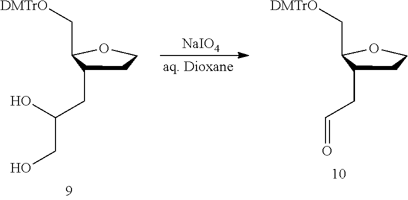

To a solution of compound 9 (65 g, 135.82 mmol, 1 eq.) in the mixed solvent of dioxane (650 mL) and H₂O (325 mL) was added NaIO₄ (58.10 g, 271.64 mmol, 15.05 mL, 2 eq.). The mixture was stirred at 15° C. for 1 hr. TLC (Petroleum ether : Ethyl acetate=0/1,Rf=0.43) indicated compound 9 was consumed completely and one new spot formed. The reaction was clean according to TLC. The reaction mixture was washed by addition H₂O 500 mL, and then extracted with Ethyl acetate 1000 mL (500 mL *2). The combined organic layers were dried over Na₂SO₄, filtered and concentrated under reduced pressure to give a residue. Compound 10 (51 g, crude) was obtained as black brown oil. ¹HNMR (400 MHz, CHLOROFORM-d) δ=9.62-9.82 (m, 1H), 7.41-7.50 (m, 1H), 7.14-7.38 (m, 8H), 6.79-6.88 (m, 4H), 3.85-3.92 (m, 1H), 3.80 (d, J=3.73 Hz, 6H), 3.10-3.24 (m, 1H), 2.56-2.68 (m, 1H), 2.38-2.55 (m, 1H), 2.24 (tt, J=12.93, 6.25 Hz, 1H), 1.60-1.69 (m, 2H), 1.29-1.44 (m, 2H), 0.89-0.97 (m, 2H).

Step 8B. Preparation of compound 11.

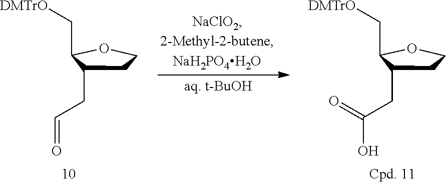

To a solution of compound 10 (51 g, 114.21 mmol, 1 eq.) in the mixed solvent of t-BuOH (500 mL) and H₂O (250 mL) added sodium;chlorite (61.98 g, 685.28 mmol, 6 eq.) sodium;dihydrogen phosphate;hydrate (63.04 g, 456.85 mmol, 4 eq.) and 2-METHYL-2-BUTENE (32.04 g, 456.85 mmol, 48.40 mL, 4 eq.). The mixture was stirred at 15° C. for 2 hr. The combined organic layers were washed with saturated NaHCO₃ solution 500 mL, dried over Na₂SO₄, filtered and concentrated under reduced pressure to give a residue. The residue was purified by column chromatography (SiO₂, Petroleum ether/Ethyl acetate=10/1 to 1/1, Dichloromethane: Methanol=10/1 to 1/1). Cpd.11 (17.1 g, 33.31 mmol, 32.78% yield, 90.11% purity) was obtained as a black brown oil. ¹HNMR (400 MHz, DMSO-d6) δ=7.17-7.42 (m, 8H), 6.88 (d, J=8.78 Hz, 3H), 3.73 (s, 6H), 3.58-3.64 (m, 1H), 2.99 (br d, J=4.64 Hz, 1H), 2.81 (q, J=6.78 Hz, 3H), 2.22-2.31 (m, 1H), 2.11-2.19 (m, 1H), 2.01-2.09 (m, 1H), 1.49-1.59 (m, 2H), 1.21-1.35 (m, 2H). LCMS (M+H⁻): 461.2; purity 90.11%. SFC: AD-3_MeOH_IPAm_10-40_Gradient_4ml_S, dr=1.02:98.98. TLC: Petroleum ether: Ethyl acetate=3:1, Rf=0.11.

WV-NU-167 & WV-NU-167-CNE (e.g., useful for WV-39402)

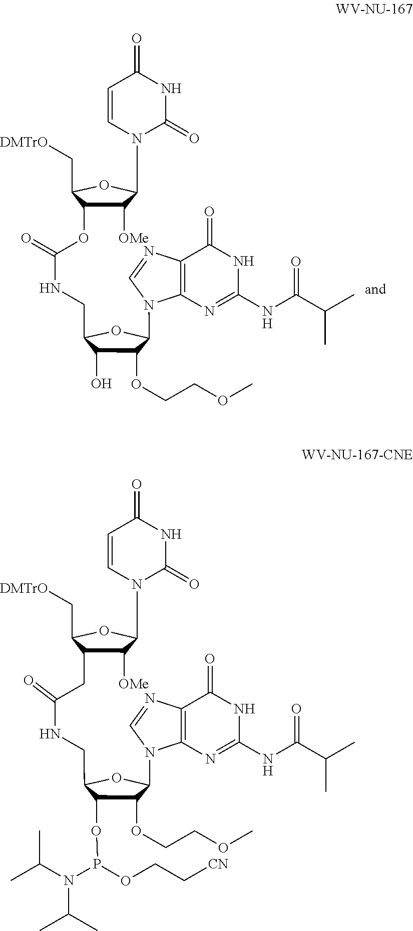

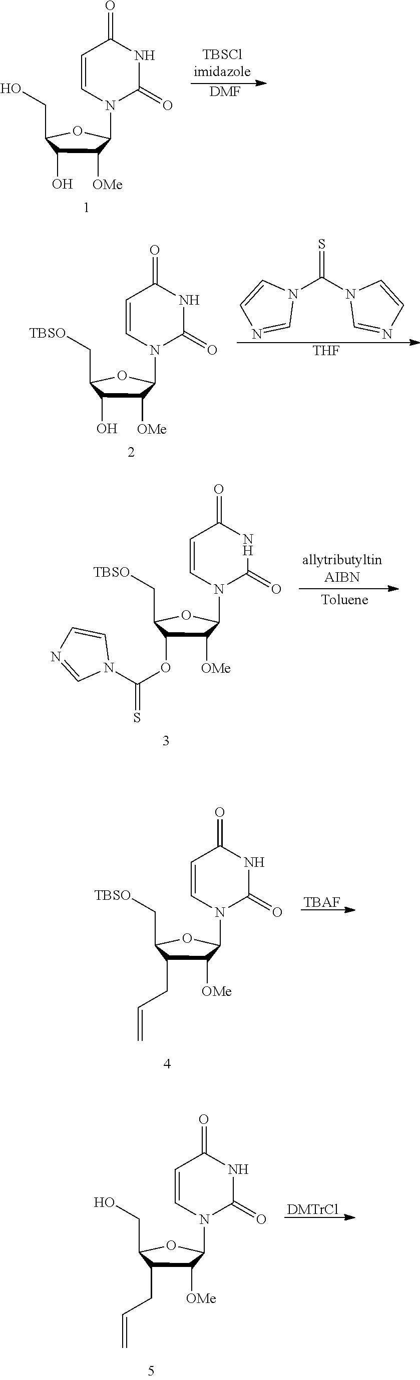

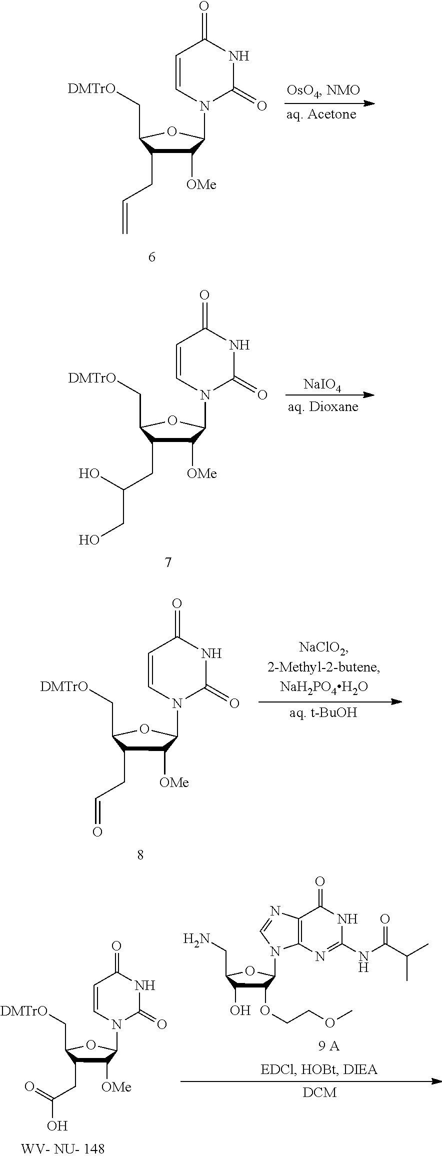

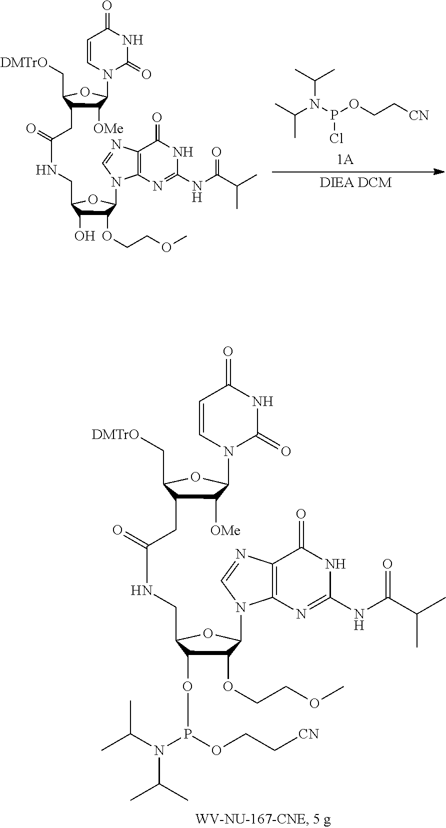

Step 1 C: Preparation of compound 2.

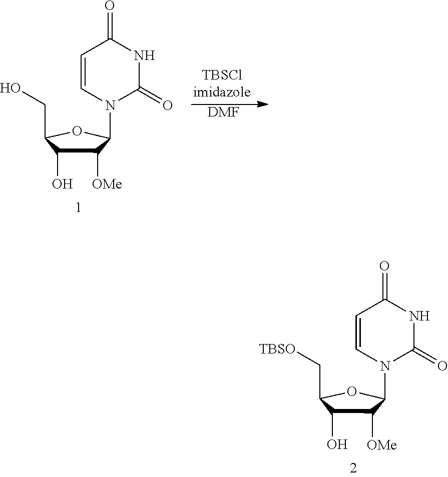

To a solution of compound 1 (85 g, 329.17 mmol, 1 eq.) in DMF (850 mL) was added imidazole (44.82 g, 658.33 mmol, 2 eq.) followed by tert-butyl-chloro-dimethyl-silane (52.09 g, 345.63 mmol, 42.35 mL, 1.05 eq.). The mixture was stirred at 15° C. for 16 hr. TLC (Petroleum ether: Ethyl acetate=1/1, Rf=0.41) compound 1 was consumed completely and one main peak with desired m/z. The reaction mixture was diluted with H₂O 250 mL and extracted with EtOAc 900m1 (300 mL *3). The combined organic was dried over Na₂SO₄, filtered and concentrated to get the crude. The residue was purified by column chromatography (SiO₂, Petroleum ether: Ethyl acetate=10:1 to 0:1) to get compound 2 (193.2 g, 518.67 mmol, 78.79% yield) as a white solid. TLC: Petroleum ether: Ethyl acetate=1:1, Rf=0.41 LCMS (M−H⁺):371.1; purity: 96.63%.

Step 2C. Preparation of compound 3.

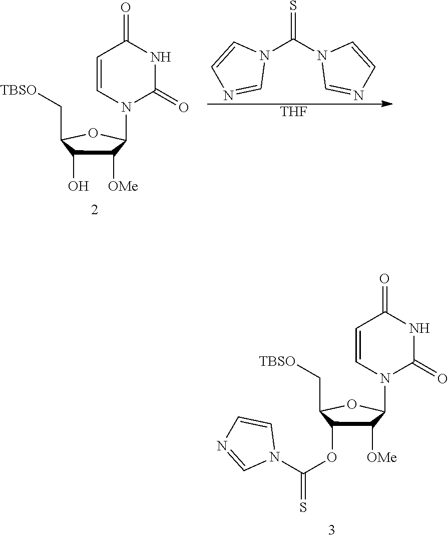

To a solution of compound 2 (193.2 g, 518.67 mmol, 1 eq.) in THF (2000 mL) was added di(imidazol-1-yl)methanethione (92.44 g, 518.67 mmol, 1 eq.) under N2. The mixture was stirred at 75° C. for 16 hr. TLC (Petroleum ether: Ethyl acetate=1:1, Rf=0.17) showed compound 2 was consumed completely and one main peak with desired m/z. The reaction mixture was concentrated under reduced pressure to give a residue. The residue was purified by column chromatography (SiO₂, Petroleum ether: Ethyl acetate=5:1 to 1:5). Compound 3 (230 g, 476.56 mmol, 92.00% yield) was obtained as a white solid. HNMR: ¹H NMR (400 MHz, CHLOROFORM-d) 6=8.40 (s, 1H), 7.90 (d, J=8.1 Hz, 1H), 7.13-7.08 (m, 3H), 6.22 (d, J=5.4 Hz, 1H), 5.86 (dd, J=3.9, 5.1 Hz, 1H), 5.76 (d, J=8.1 Hz, 1H), 4.48-4.45 (m, 1H), 4.16 (t, J=5.2 Hz, 1H), 4.06-3.93 (m, 2H), 3.47-3.42 (m, 3H), 0.96 (s, 9H), 0.19-0.12 (m, 6H). LCMS (M−H⁺):481.1; purity: 93.02%. TLC: Petroleum ether: Ethyl acetate=1:1, Rf=0.17.

Step 3C. Preparation of 4.

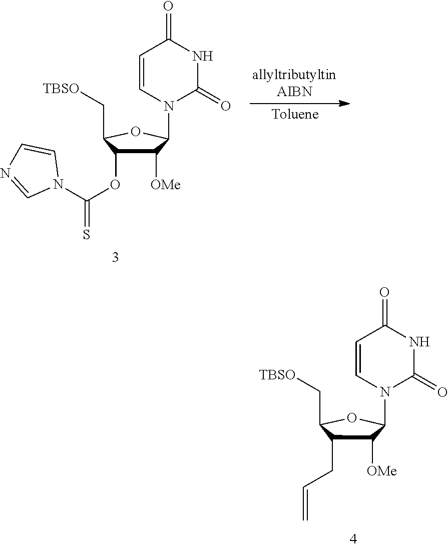

To a solution of compound 3 (90 g, 186.48 mmol, 1 eq.) in toluene (1000 mL) was added AIBN (21.44 g, 130.54 mmol, 0.7 eq.) and allyl(tributyl)stannane (279.55 g, 844.25 mmol, 258.84 mL, 4.53 eq.) in N2. The mixture was stirred at 110° C. for 16 hr. TLC (Petroleum ether: Ethyl acetate=0:1, Rf=0.67). The reaction mixture was quenched by addition H₂O (1000 mL), and extracted with EtOAc (1000 mL * 3). The combined organic layers were concentrated under reduced pressure to give a residue and the solvent was quenched by sat. KF (aq.) 1500 mL. The residue was purified by column chromatography (SiO₂, Petroleum ether: Ethyl acetate=30:1 to 0:1). Compound 4 (37 g, 93.30 mmol, 55.22% yield) was obtained as a yellow oil. LCMS (M−H⁺):395.1; purity: 55.74%. TLC: Petroleum ether: Ethyl acetate=0:1, Rf=0.68.

Step 4C Preparation of compound 5.

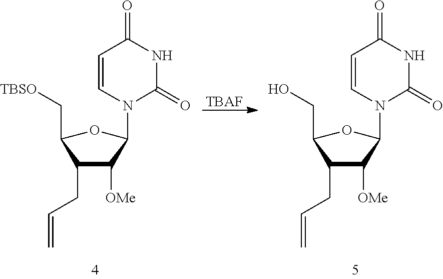

To a solution of compound 4 (37 g, 93.30 mmol, 1 eq.) in THF (400 mL) was added TBAF (1 M, 139.96 mL, 1.5 eq.). The mixture was stirred at 15° C. for 2 hr. TLC (Petroleum ether: Ethyl acetate=1:1, Rf=0.23) The reaction mixture was concentrated under reduced pressure to give a residue. The residue was purified by column chromatography (SiO₂, Petroleum ether: Ethyl acetate=15:1 to 1:2). Compound 5 (9 g, 31.88 mmol, 34.17% yield) was obtained as a yellow oil. LCMS (M−H⁺): 281.1; purity: 75.39%. TLC: Petroleum ether: Ethyl acetate=1:1, Rf=0.23).

Step 5C. Preparation of compound 6.

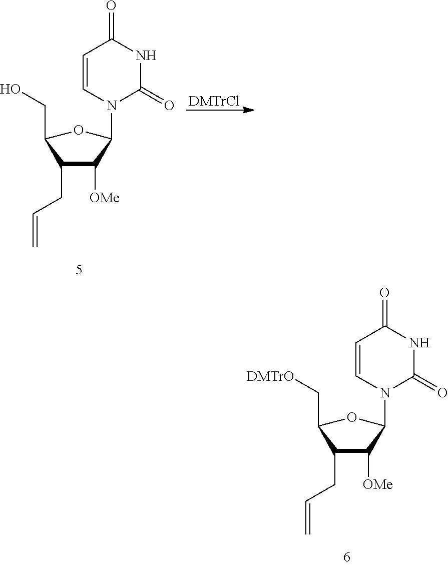

To a solution of compound 5 (9 g, 31.88 mmol, 1 eq.) in PYRIDINE (90 mL) was added DMT-Cl (12.96 g, 38.26 mmol, 1.2 eq.). The mixture was stirred at 15° C. for 12 hr. (Petroleum ether: Ethyl acetate=1:1, Rf=0.43) The reaction mixture was concentrated under reduced pressure to remove pyridine. The combined organic layers were washed with H₂O 120 mL (40 mL * 3), dried over Na₂SO₄, filtered and concentrated under reduced pressure to give a residue. The residue was purified by column chromatography (SiO₂, Petroleum ether: Ethyl acetate=50:1 to 0:1). Compound 6 (11.8 g, 20.18 mmol, 65.56% yield) was obtained as a yellow solid. LCMS (M−H⁺):583.2; purity: 78.06%. TLC: Petroleum ether: Ethyl acetate=1:1, Rf=0.44.

Step 6C. Preparation of compound 7.

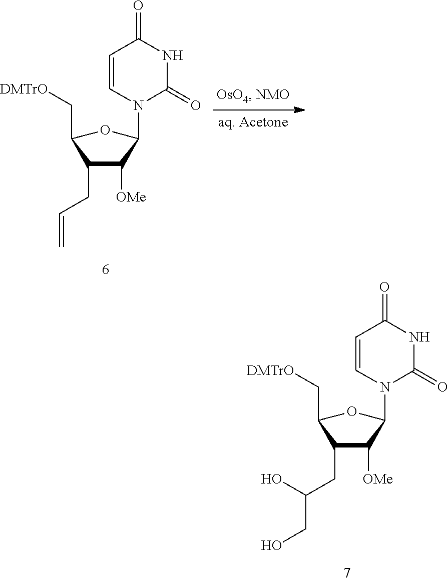

To a solution of compound 6 (11.8 g, 20.18 mmol, 1 eq.) in the mixed solvent of ACETONE (300 mL) and H₂O (30 mL) was added OsO₄ (0.75 g, 2.95 mmol, 153.06 uL, 1.46e-1 eq.) and NMO (2.60 g, 22.20 mmol, 2.34 mL, 1.1 eq.). The mixture was stirred at 15° C. for 3 hr. TLC (Petroleum ether: Ethyl acetate=0:1, Rf=0.17). The reaction mixture was quenched by addition Na₂SO_{3 300} ml at 0° C., and then extracted with ethyl acetate 500 mL (250 mL * 2). The combined organic layers were dried over Na₂SO₄, filtered and concentrated under reduced pressure to give a residue. Compound 7 (12 g, crude) was obtained as a yellow oil. LCMS (M−H⁺):617.2; purity: 92.16%. TLC: Petroleum ether: Ethyl acetate=0:1, Rf=0.17.

Step 7C. Preparation of compound 8.

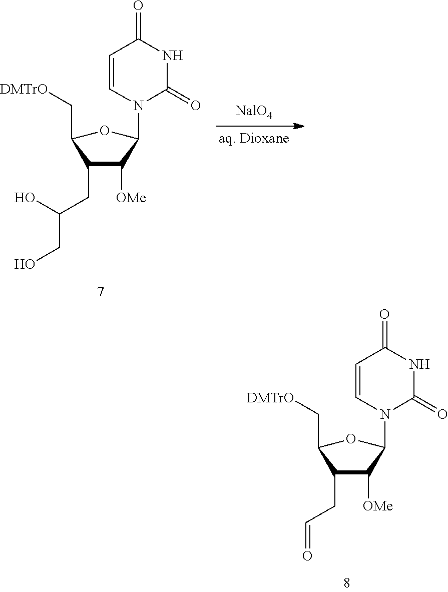

To a solution of compound 7 (12 g, 19.40 mmol, 1 eq.) in the mixed solvent of dioxane (120 mL) H₂O (60 mL) was added NaIO₄ (8.30 g, 38.79 mmol, 2.15 mL, 2 eq.). The mixture was stirred at 15° C. for 1 hr. The reaction mixture was washed by addition H₂O 400 mL, and then extracted with Ethyl acetate 600 mL (300 mL *2). The combined organic layers were dried over Na₂SO₄, filtered and concentrated under reduced pressure to give a residue. Compound 8 (11 g, crude) was obtained as a white solid. LCMS (M−H⁺):585.1.

8. Preparation of compound WV-NU-148.

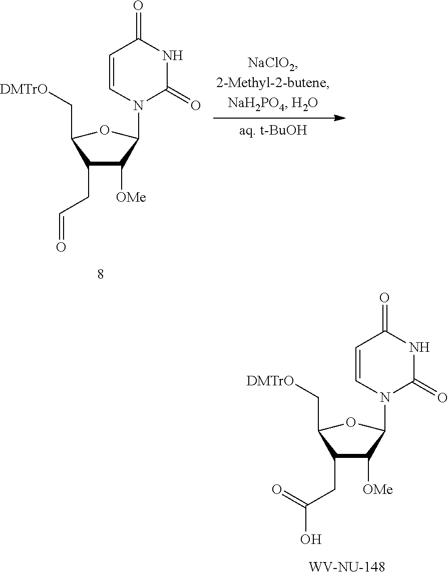

To a solution of compound 8 (11 g, 18.75 mmol, 1 eq.) in the mixed solvent of t-BuOH (110 mL) and H₂O (60 mL), then added 2-methylbut-2-ene (5.26 g, 75.00 mmol, 7.95 mL, 4 eq.) sodium;chlorite (10.18 g, 112.51 mmol, 6 eq.) and sodium dihydrogen phosphate hydrate (10.35 g, 75.00 mmol, 4 eq.). The mixture was stirred at 15° C. for 2 hr. TLC (Dichloromethane: Methanol=5:1, Rf=0.14) The reaction mixture was extracted with Ethyl acetate 300 mL (100 mL * 3). The combined organic layers were washed with NaHCO3 200 mL, dried over Na₂SO₄, filtered and concentrated under reduced pressure to give a residue. The residue was purified by column chromatography (SiO₂, Petroleum ether: Ethyl acetate=5:1 to 0:1 to Ethyl acetate: MeOH=10:1, 5%TEA). Compound WV-NU-148 (7.3 g, 12.11 mmol, 64.60% yield) was obtained as a white solid. HNMR: ¹H NMR (400 MHz, CHLOROFORM-d) δ ppm 8.18 (br d, J=8.00 Hz, 1H), 7.42 (br d, J=7.50 Hz, 2H), 7.20-7.34 (m, 9H), 6.84 (br d, J=7.88 Hz, 4H), 5.89 (s, 1H), 5.30 (br d, J=8.13 Hz, 1H), 3.98-4.07 (m, 2H), 3.79 (s, 6H), 3.52 (s, 2H), 2.99 (q, J=7.25 Hz, 5H), 2.71 (br s, 1H), 2.46 (br dd, J=16.26, 10.51 Hz, 1H), 2.09 (br s, 1H). LCMS (M−H⁺):601.2; purity: 90.26%. TLC: Dichloromethane: Methanol=5:1, Rf=0.14.

Step 9C. Preparation of compound WV-NU-167.

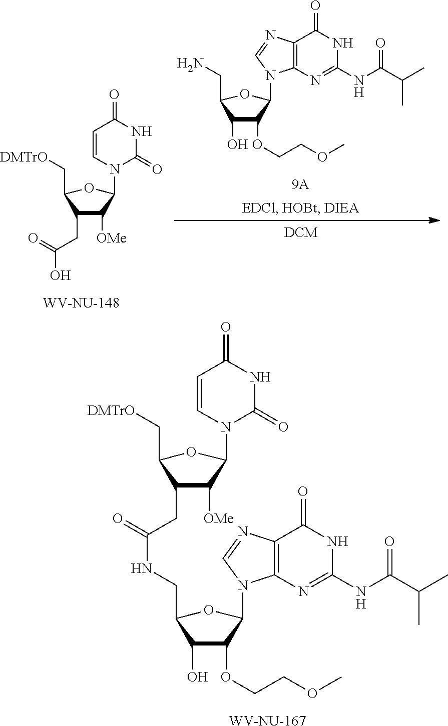

To a solution of compound WV-NU-148 (5.3 g, 8.79 mmol, 1 eq) in DCM (60 mL) was added EDCI (3.37 g, 17.59 mmol, 2 eq), HOBt (2.38 g, 17.59 mmol, 2 eq), DIEA (2.27 g, 17.59 mmol, 3.06 mL, 2 eq) and compound 9A (4.33 g, 10.55 mmol, 1.2 eq). The mixture was stirred at 15° C. for 12 hr. The reaction mixture was diluted with H₂O 100 mL and extracted with DCM 100 mL * 3. The combined organic layers were dried over Na₂SO₄, filtered and concentrated under reduced pressure to give a residue. The residue was purified by column chromatography (SiO₂, Petroleum ether/Ethyl acetate=1/1 to 0/1, then Ethyl acetate/methanol=10/1 to 8/1) to get WV-NU-167 (4 g, 3.66 mmol, 41.60% yield, 91.02% purity) as a yellow solid. ¹HNMR (400 MHz, DMSO-d₆) 6=12.09 (br s, 1H), 11.62 (s, 1H), 11.38 (br s, 1H), 8.27 (s, 1H), 8.08 (br s, 1H), 7.93 (d, J=8.13 Hz, 1H), 7.28-7.40 (m, 4H), 7.19-7.27 (m, 5H), 6.82-6.95 (m, 4H), 5.84-5.92 (m, 1H), 5.70-5.77 (m, 1H), 5.19 (br d, J=4.63 Hz, 1H) , 5.09 (d, J=8.00 Hz, 1H), 4.37-4.45 (m, 1H), 4.23 (br d, J=3.13 Hz, 1H) , 3.88-3.96 (m, 2H), 3.81 (d, J=5.13 Hz, 1H), 3.73 (d, J=1.88 Hz, 6H), 3.63-3.69 (m, 1H), 3.50-3.58 (m, 1H), 3.38-3.42 (m, 3H), 3.35-3.36 (m, 5H), 3.17 (br d, J=3.25 Hz, 4H), 2.65-2.84 (m, 2H), 2.28-2.41 (m, 1H), 6=1.12 (dd, J=6.75, 2.50 Hz, 6H). LCMS: (M−H+):993.3 TLC (Ethyl acetate: Methanol=5:1, Rf=0.02).

Step 10C. Preparation of compound WV-NU-167-CNE.

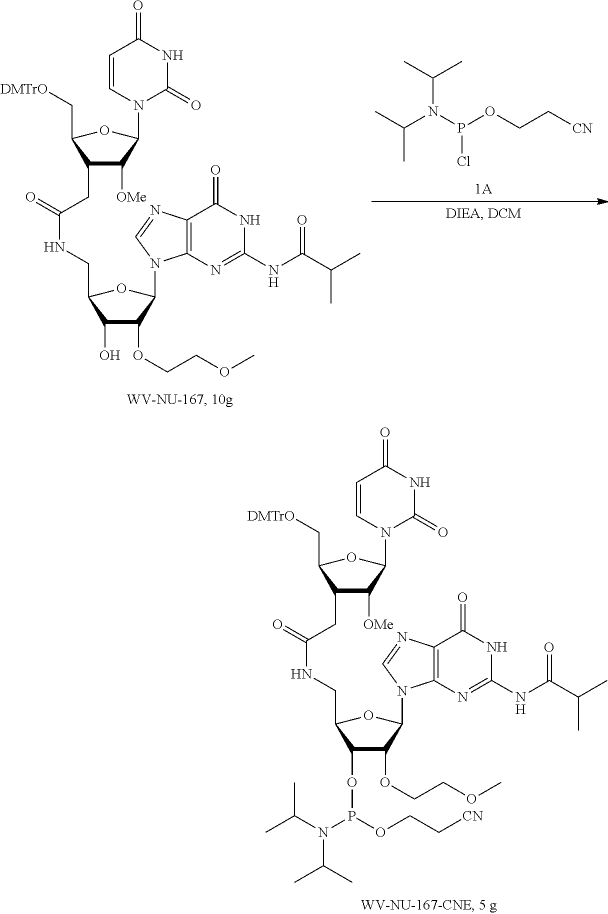

Compound WV-NU-167 (4 g, 4.02 mmol, 1 eq) was dried by azeotropic distillation on a rotary evaporator with toluene (10 mL*3). To a solution of compound WV-NU-167 (4 g, 4.02 mmol, 1 eq) in DCM (500 mL) was added DIEA (1.56 g, 12.06 mmol, 2.10 mL, 3 eq) then 3-[chloro-(diisopropylamino)phosphanyl]oxypropanenitrile (1.90 g, 8.04 mmol, 2 eq). The mixture was stirred at 15° C. for 2 hr. TLC (Ethyl acetate: Methanol=10:1, Rf=0.43) indicated compound WV-NU-167 was consumed and one new spot formed. The reaction mixture was quenched by addition sat. NaHCO3 aq. (10 mL) at 0° C., and extracted with DCM (20 mL*3), dried over Na₂SO₄, filtered and concentrated under reduced pressure to give a residue. The residue was purified by column chromatography (SiO₂, Petroleum ether/Ethyl acetate=1/0 to 0/1, then EtOAc/ACN=1/0 to 1/1, 5% TEA) to get compound WV-NU-167-CNE (3 g, 2.51 mmol, 62.44% yield) as a white solid. ¹HNMR (400 MHz, DMSO-d6) δ=12.12 (br s, 1H), 11.69-11.54 (m, 1H), 11.48-11.31 (m, 1H), 8.39-8.28 (m, 1H), 8.20-8.08 (m, 1H), 7.99-7.87 (m, 1H), 7.45-7.30 (m, 4H), 7.26 (br d, J=8.4 Hz, 5H), 6.98-6.83 (m, 4H), 5.88 (dd, J=7.4, 11.7 Hz, 1H), 5.77 (s, 1H), 5.15-5.07 (m, 1H), 4.74-4.59 (m, 1H), 4.54-4.42 (m, 1H), 4.03-3.90 (m, 2H), 3.87-3.79 (m, 3H), 3.76-3.69 (m, 7H), 3.65-3.42 (m, 5H), 3.40-3.36 (m, 5H), 3.17-3.12 (m, 3H), 2.83-2.66 (m, 4H), 2.45-2.31 (m, 1H), 1.20-1.16 (m, 10H), 1.14 (br dd, J=2.6, 6.6 Hz, 9H). ³¹PNMR (162 MHz, DMSO-d6) δ=149.63 (s, 1P), 149.52 (s, 1P), 149.47 (s, 1P), 13.88 (s, 1P).

Those skilled in the art appreciate that compounds like WV-NU-167, WU-NU-161A, WV-NU-173, WV-NU-174, etc., can alternatively be coupled with chiral auxiliary reagents

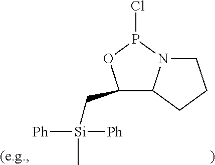 [SMILES: C[Si](C[C@H]1OP(Cl)N2CCCC12)(c1ccccc1)c1ccccc1]

to provide compounds useful for stereoselective construction of linkages.

WV-NU-173 and WV-NU-173-CEP.

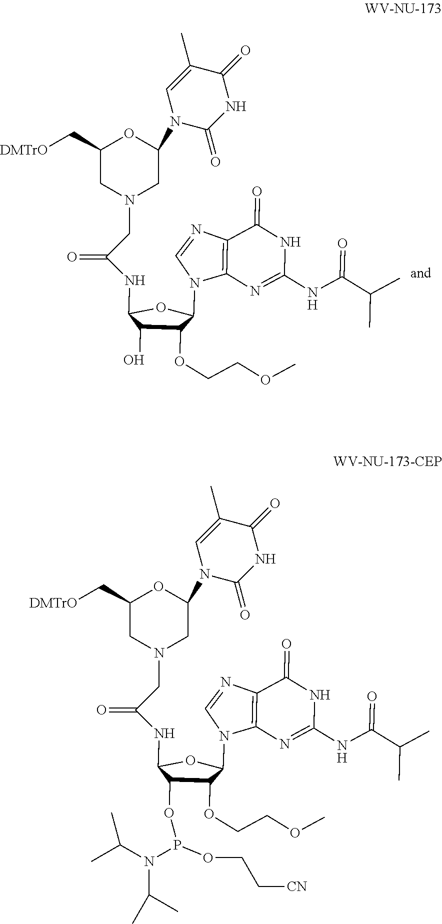

General synthetic procedure:

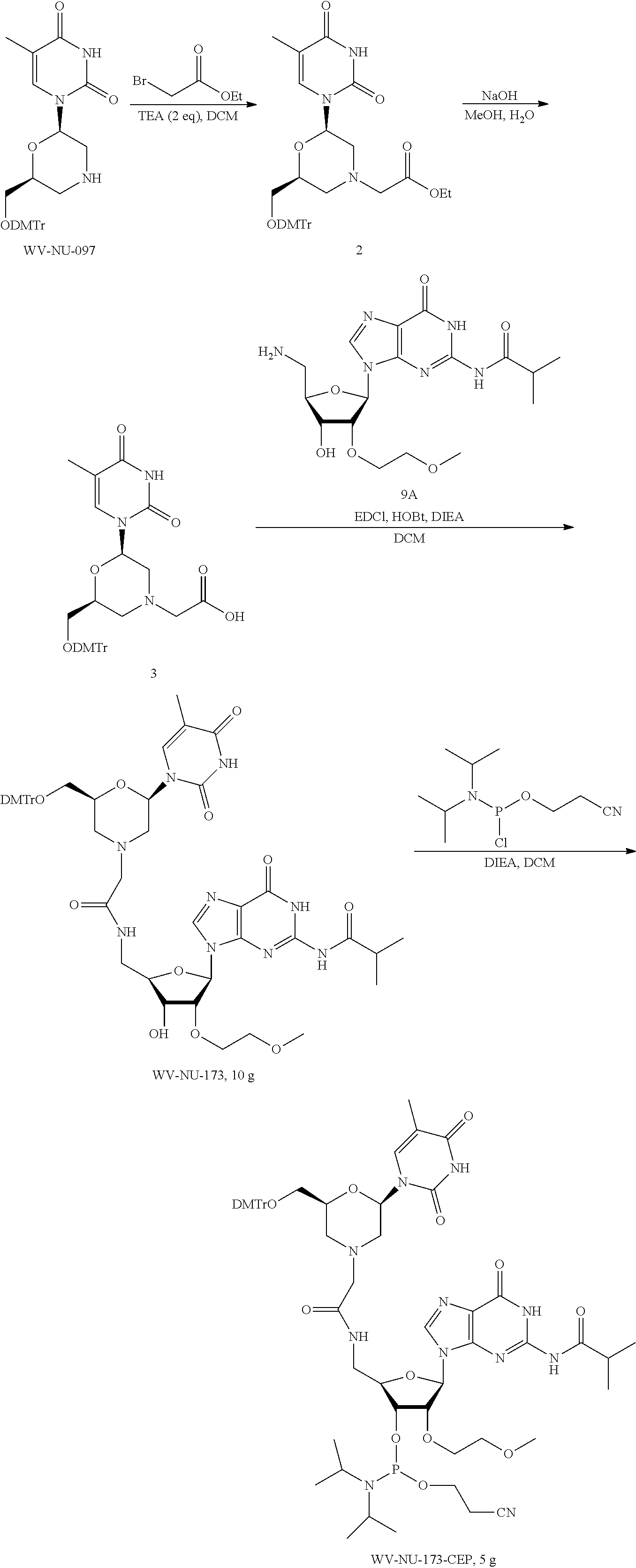

Step 1D. Preparation of compound 2.

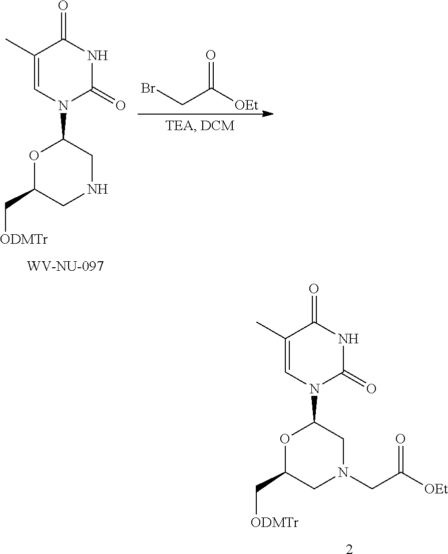

To a solution of compound WV-NU-097 (25 g, 45.99 mmol, 1 eq.) in DCM (250 mL) were added ethyl 2-bromoacetate (11.52 g, 68.98 mmol, 7.63 mL, 1.5 eq.) and TEA (9.31 g, 91.98 mmol, 12.80 mL, 2 eq.). The mixture was stirred at 15° C. for 16 hr. TLC (Petroleum ether: Ethyl acetate=1:1, Rf=0.54) indicated Reactant 1 was consumed completely and one new spot formed. The reaction mixture was quenched by addition NaHCO₃ 200 mL, The combined organic layers were washed with brine 200 mL, dried over Na₂SO₄ filtered and concentrated under reduced pressure to give a residue. Compound 2 (28 g, crude) was obtained as a yellow oil. LCMS (M−H⁺):628.2; purity: 62.35%. TLC: Petroleum ether: Ethyl acetate=1:1, Rf=0.54.

Steep 2D. Preparation of compound 3.

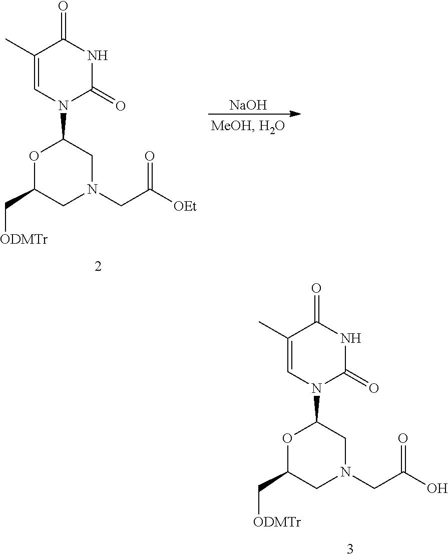

To a solution of compound 2 (28 g, 44.47 mmol, 1 eq.) in MeOH (300 mL) was added NaOH (2 M, 44.47 mL, 2 eq.) and H₂O (40 mL). The mixture was stirred at 15° C. for 5 hrThe reaction mixture was concentrated under reduced pressure to remove Methanol. The residue was extracted with Ethyl acetate (100 * 3 mL). The combined organic layers were dried over Na₂SO₄, filtered and concentrated under reduced pressure to give a residue. The crude product was purified by reversed-phase HPLC (column: C18 20-35 um 100A 64 g; mobile phase: [water-MeOH]; B%: 0%-60% @ 50 mL/min). Compound 3 (16 g, 25.61 mmol, 57.61% yield, Na) was obtained as a white solid. LCMS (M−H+):600.2; purity: 99.39%.

Step 3D. Preparation of compound 9A.

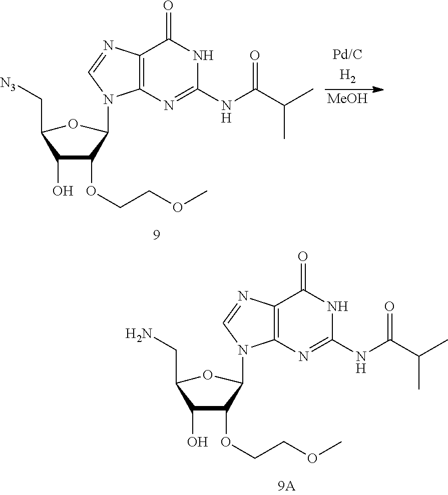

A mixture of compound 9 (9 g, 20.62 mmol, 1 eq.), Pd/C (458.27 umol, 50% purity) in MeOH (110 mL)was degassed and purged with H2 for 3 times, and then the mixture was stirred at 15° C. for 1 hr under H2 (41.57 mg, 20.62 mmol, 1 eq.) (15 psi). TLC (Ethyl acetate: Methanol=5:1, Rf=0.04) indicated compound 9 was consumed completely and one new spot formed. The reaction was clean according to TLC. The mixture was filtered and the filtrated was concentrated to get the crude. Compound 9A (8.2 g, crude) was obtained as a white oil (10.1 g, 20.30 mmol, 50.30% yield) was obtained as a white solid. TLC: Ethyl acetate: Methanol=5:1, Rf=0.04.

Step 4D. Preparation of compound WV-NU-173.

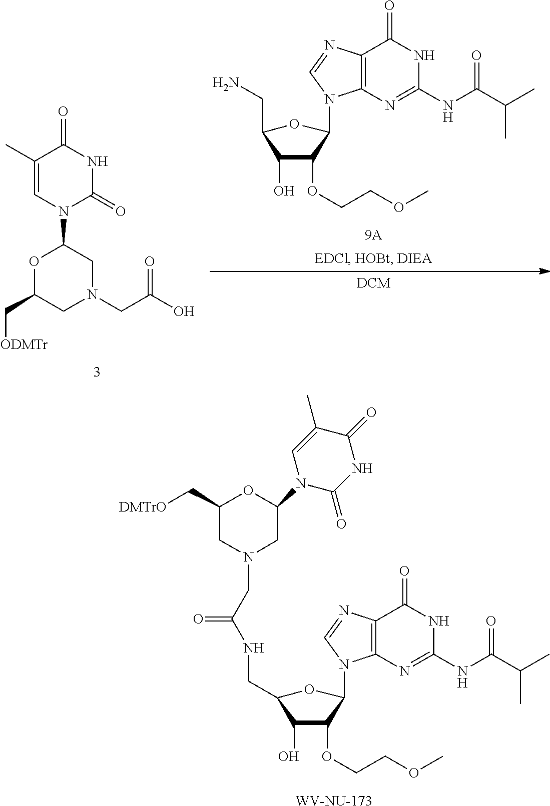

To a solution of compound 3 (8.92 g, 14.82 mmol, 1 eq.) in DCM (100 mL) was added EDCI (5.68 g, 29.64 mmol, 2 eq.), HOBt (4.01 g, 29.64 mmol, 2 eq.), DIEA (3.83 g, 29.64 mmol, 5.16 mL, 2 eq.) and compound 9A (7.3 g, 17.79 mmol, 1.2 eq.). The mixture was stirred at 15° C. for 12 hr. TLC (Ethyl acetate: Methanol=6:1, Rf=0.04) indicated compound 3 was consumed completely and one new spot formed. The reaction mixture was diluted with H₂O 50 mL and extracted with DCM (100 mL * 3). The combined organic layers were dried over Na₂SO₄, filtered and concentrated under reduced pressure to give a residue. The residue was purified by column chromatography (SiO₂, Ethyl acetate: Methanol=1:0 to 4:1, 5% TEA, PE. Compound WV-NU-173 (4 g, 4.02 mmol, 25.00% yield) was obtained as a white solid. ¹HNMR (400 MHz, DMSO-d6) δ=12.08 (br s, 1H), 11.61 (br s, 1H), 11.38 (s, 1H), 8.26-8.32 (m, 1H), 8.14 (br t, J=5.88 Hz, 1H), 7.50 (s, 1H), 7.17-7.39 (m, 9H), 6.86 (d, J=8.00 Hz, 4H), 5.88 (d, J=6.50 Hz, 1H), 5.71-5.77 (m, 2H), 5.18 (d, J=4.88 Hz, 1H), 4.36-4.42 (m, 1H), 4.23-4.30 (m, 1H), 3.63-3.76 (m, 7H), 3.51-3.57 (m, 1H), 3.36-3.49 (m, 5H), 3.12-3.16 (m, 3H), 3.04-3.10 (m, 3H), 2.70-2.91 (m, 4H), 2.35 (br t, J=10.38 Hz, 1H), 2.10 (br t, J=10.82 Hz, 1H), 1.76 (s, 3H), 1.11 (dd, J=6.75, 2.38 Hz, 7H). LCMS (M−H⁺): 922.3; purity: 98.06% TLC: Ethyl acetate: Methanol=6:1, Rf=0.04.

Step 5D. Preparation of compound WV-NU-173-CEP.

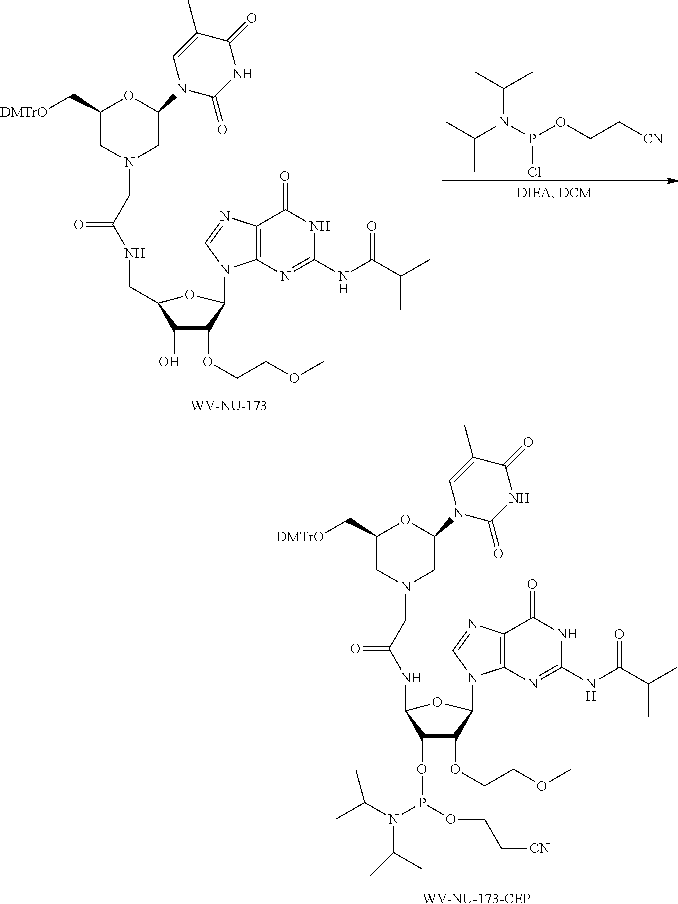

Compound WV-NU-173 (1.6 g, 1.61 mmol, 1 eq.) was dried by azeotropic distillation on a rotary evaporator with toluene (10 mL*3). To a solution of compound WV-NU-173 (1.6 g, 1.61 mmol, 1 eq.), 4A MS (2 g, 1.61 mmol, 1 eq.) in DCM (40 mL) was added DIEA (624.08 mg, 4.83 mmol, 841.07 uL, 3 eq.), and then3[chloro-(diisopropylamino)phosphanyl]oxypropanenitrile (761.90 mg, 3.22 mmol, 2 eq.) was added. The mixture was stirred at 20° C. for 2 hr. TLC (Ethyl acetate: Methanol=5:1, Rf=0.43) showed most of the reactant 1 was disappeared and the desired spot was found. The mixture was poured into the ice-sat. NaHCO₃ (aq, 10 mL) and extracted with DCM (10 mL*3), the combined organic was dried over Na₂SO₄, filtered and concentrated to get the crude. The residue was purified by MPLC (SiO₂, Petroleum ether/Ethyl acetate=1/0 to 0/1, then EtOAc/CAN=1/0 to 1/1, 5% TEA) to get WV-NU-173-CEP (0.5 g, 418.67 umol, 26.01% yield) as a yellow solid. ¹HNMR (400 MHz, DMSO-d6) δ=12.12 (br s, 1H), 11.66-11.52 (m, 1H), 11.44-11.31 (m, 1H), 8.43-8.30 (m, 1H), 8.28-8.16 (m, 1H), 7.54-7.45 (m, 1H), 7.42-7.35 (m, 2H), 7.33-7.18 (m, 7H), 6.88 (br d, J=8.5 Hz, 4H), 5.94-5.74 (m, 2H), 5.09-4.74 (m, 1H), 4.73-4.48 (m, 1H), 4.31-4.20 (m, 1H), 3.79-3.68 (m, 7H), 3.65-3.47 (m, 4H), 3.08 (br s, 2H), 2.93-2.71 (m, 5H), 2.42-2.31 (m, 1H), 2.23-2.10 (m, 1H), 1.13 (br dd, J=2.4, 6.7 Hz, 9H). ³¹PNMR (162 MHz, DMSO-d₆) δ=149.67 (s, 1P), 149.44 (s, 1P), 13.91 (s, 1P), 7.14 (s, 1P).

WV-NU-174 and WV-NU-174-CEP (e.g., useful for WV-40835)

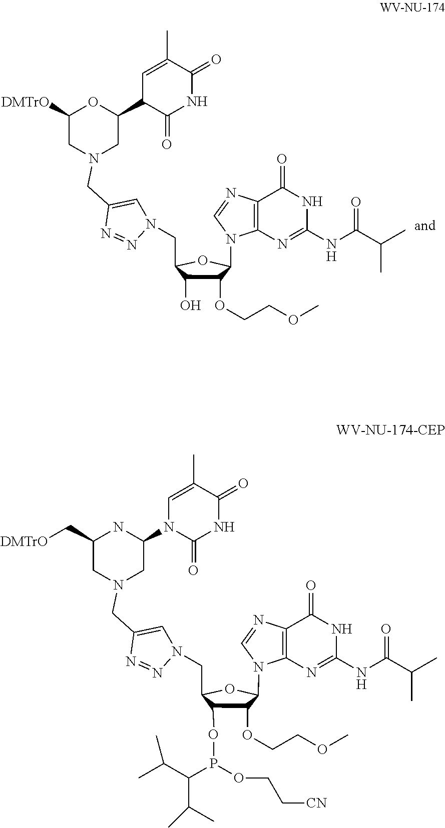

General synthetic route:

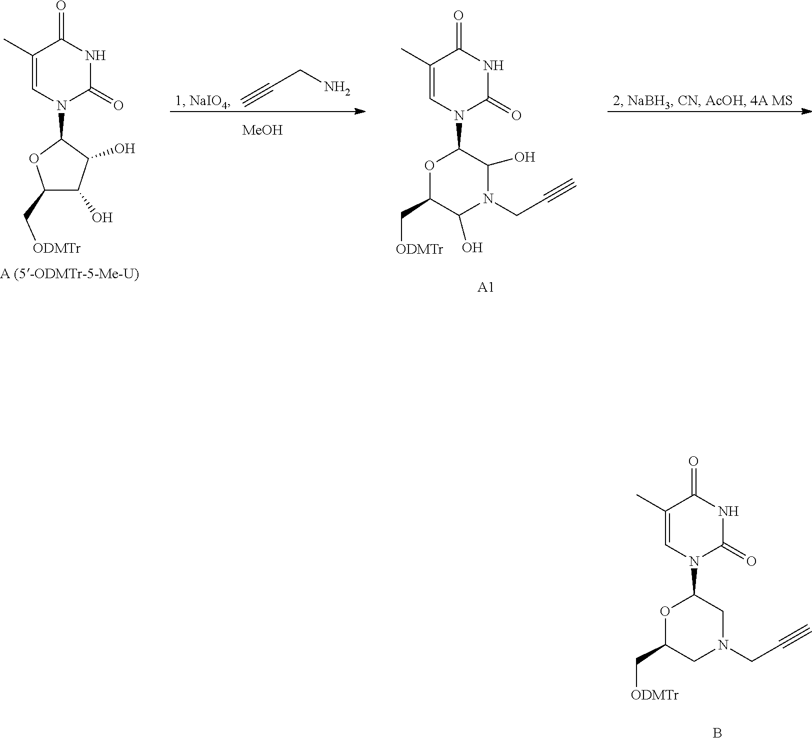

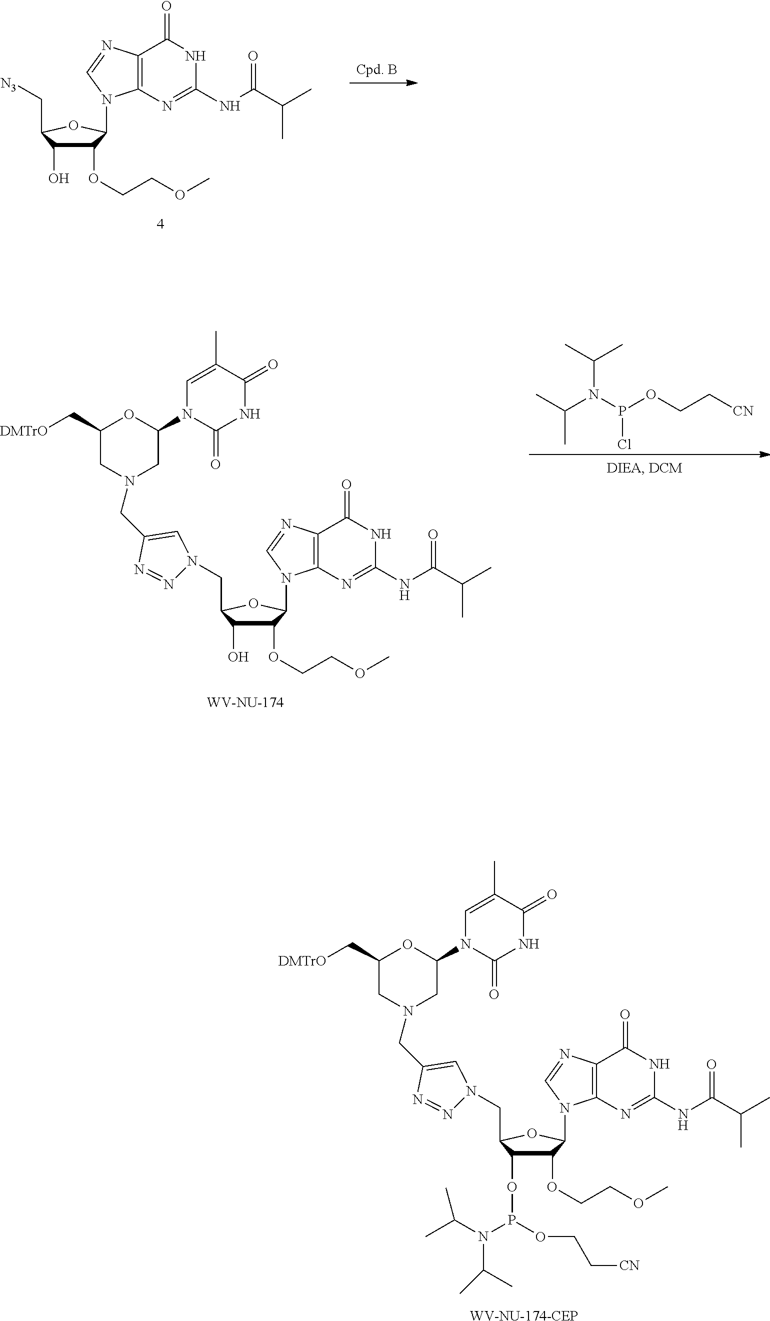

Step 1E. Preparation of compound B.

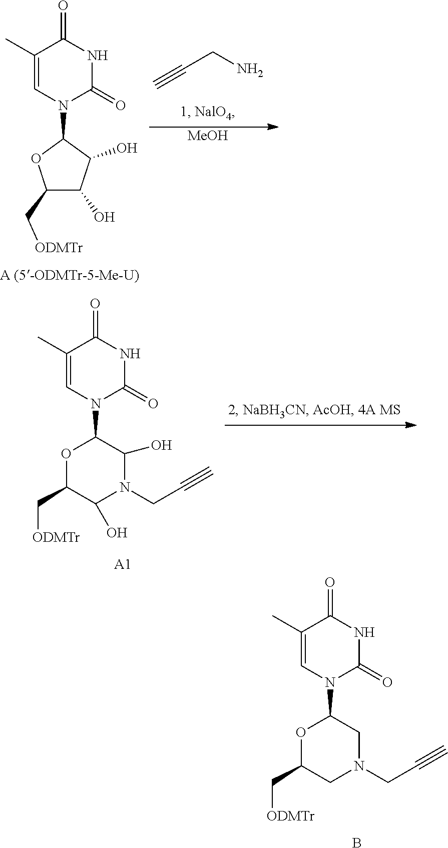

For two batches: To a stirred solution of compound A (25 g, 44.60 mmol, 1 eq.) in MeOH (300 mL) under N2 atmosphere, was added a solution of NaIO₄ (10.49 g, 49.06 mmol, 2.72 mL, 1.1 eq.) in H₂O (75 mL) drop wise, followed by prop-2-yn-1-amine (3.07 g, 55.74 mmol, 3.57 mL, 1.25 eq.) in one portion. The resulting solution was stirred at 15° C. for 3 hours. TLC(Petroleum ether : Ethyl acetate=0:1,Rf=0.41) The two batches of solution was stirred at 15° C. for 3.08 hours, during which time a white precipitate formed, the mixture was filtered. Compound A1 (54 g, crude) was obtained as a white solid. TLC: Petroleum ether: Ethyl acetate=0:1, Rf=0.41. LCMS (M−H⁺):612.2; purity: 50%. For two batches: To the stirred solution of the Al (27 g, 44.00 mmol, 1 eq.) in the mixed solvents of H₂O (75 mL) and MeOH (300 mL) was added NaBH₃CN (5.53 g, 88.00 mmol, 2 eq.) followed by the drop wise addition of AcOH (3.96 g, 66.00 mmol, 3.77 mL, 1.5 eq.). The reaction was stirred for 12 hr at 15° C. TLC (Petroleum ether: Ethyl acetate=1:1, Rf=0.36) indicated compound Al was consumed completely and one new spot formed. The reaction was clean according to TLC. The mixture of two batches of solution and the volatile organic were removed by evaporation. The residue was partitioned between sat. NaHCO3 500 mL and EtOAc 500 mL, the aqueous layer was extracted with EtOAc 500 mL. The combined organic layers were washed with brine (3 * 200 mL), dried over Na₂SO₄, and evaporated. The residue was purified by column chromatography (SiO2, Petroleum ether: Ethyl acetate=10:1 to 1:2). TLC (Petroleum ether: Ethyl acetate=1:1, Rf=0.33). Compound B (28 g, 48.14 mmol, 56.00% yield) was obtained as a white solid. TLC: Petroleum ether: Ethyl acetate=1:1, Rf=0.33 LCMS (M−H⁺):580.2; purity: 95.86%.

Step 2E. Preparation of compound WV-NU-174.

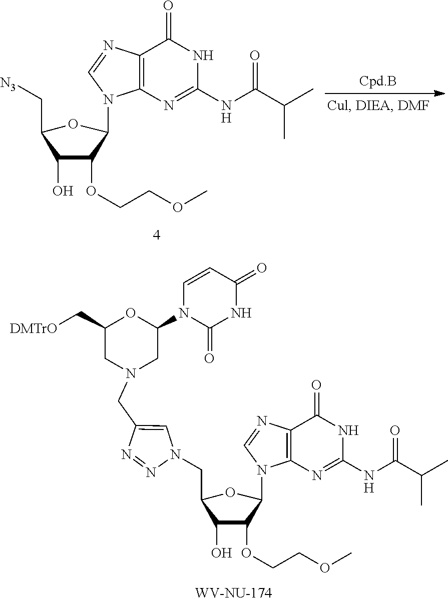

To a solution of compound 4 (2.4 g, 5.50 mmol, 0.8 eq.) and Cpd.B (4.00 g, 6.87 mmol, 1 eq.) and DIEA (1.78 g, 13.75 mmol, 2.39 mL, 2 eq.) in DMF (50 mL) was added iodocopper (261.83 mg, 1.37 mmol, 0.2 eq.) in N2. The mixture was stirred at 15° C. for 12 hr. The reaction mixture was diluted with water 100 mL, extracted with DCM (50 mL * 5) and concentrated under reduced pressure to give a residue. The crude residue were purified by column chromatography (SiO₂, Petroleum ether:Ethyl acetate=1:0to 0:1 to Ethyl acetate: MeOH=10:1). Compound WV-NU-174 (13 g, 12.77 mmol, 43.33% yield) was obtained as a white solid. ¹HNMR (400 MHz, DMSO-d6) δ=12.10 (br s, 1H), 11.58 (br s, 1H), 11.36 (s, 1H), 8.16 (s, 1H), 7.96 (s, 1H), 7.50 (s, 1H), 7.18-7.39 (m, 10H), 6.87 (br d, J=8.50 Hz, 4H), 5.91 (br d, J=5.00 Hz, 1H), 5.59 (dd, J=9.82, 2.19 Hz, 1H), 5.44 (br d, J=4.50 Hz, 1H), 4.65-4.78 (m, 2H), 4.65-4.78 (m, 2H), 4.65-4.78 (m, 2H), 4.65-4.78 (m, 2H), 4.65-4.78 (m, 2H), 4.65-4.78 (m, 2H), 4.65-4.78 (m, 2H), 4.65-4.78 (m, 2H), 4.65-4.78 (m, 2H), 4.65-4.78 (m, 2H), 4.39 (br d, J=3.38 Hz, 2H), 4.27 (br s, 1H), 3.89-3.97 (m, 1H), 3.64-3.77 (m, 9H), 3.52-3.61 (m, 2H), 3.05-3.17 (m, 5 H), 2.86-2.96 (m, 2H), 2.70-2.84 (m, 2H), 2.17 (br t, J=10.32 Hz, 1H), 1.76 (s, 3H), 1.11 (dd, J=6.75, 1.88 Hz, 6H) LCMS (M−H+):1016.3; purity: 96.82%. TLC: (Ethyl acetate : Methanol=5:1), Rf=0.27.

Step 2 F. Preparation of compound WV-NU-174-CEP.

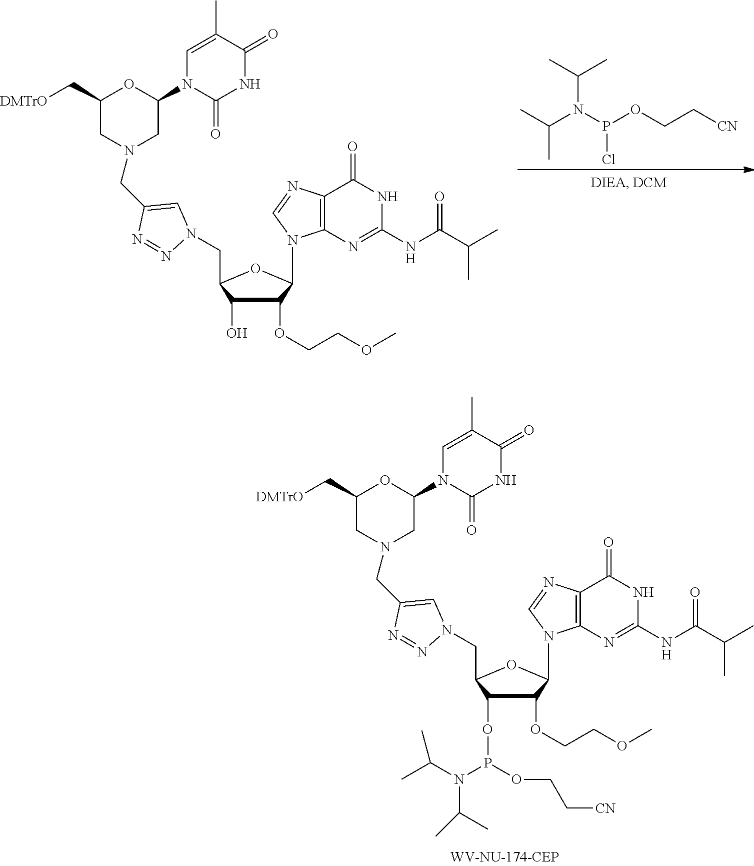

Compound WV-NU-174 (6 g, 5.89 mmol, 1 eq.) was dried by azeotropic distillation on a rotary evaporator with toluene (30 mL*3). To a solution of compound WV-NU-174 (6 g, 5.89 mmol, 1 eq.) in DCM (80 mL) was added DIEA (2.29 g, 17.68 mmol, 3.08 mL, 3 eq.) and then 3[chloro-(diisopropylamino)phosphanyl]oxypropanenitrile (2.79 g, 11.79 mmol, 2 eq.). The mixture was stirred at 20° C. for 2 hr. TLC (Dichloromethane: Methanol=15:1, Rf=0.43) indicated WV-NU-174 was consumed and one new spot formed. The reaction mixture was quenched by addition sat. NaHCO3 aq. (20 mL) at 0° C., and extracted with DCM (30 mL*3), dried over Na₂SO₄, filtered and concentrated under reduced pressure to give a residue. The residue was purified by column chromatography (SiO₂, Petroleum ether/Ethyl acetate=1/0 to 0/1, then EtOAc/ACN=1/0 to 1/1, 5% TEA) to get compound WV-NU-174-CEP (5.2 g, 4.27 mmol, 72.42% yield) as a white solid. HNMR: ET5957-1796—P1A1, PNMR: ET5957-1796—P1A1, LCMS: ET5957-1796—P1B1. ¹HNMR (400 MHz, DMSO-d6) δ=12.15 (br s, 1H), 11.58 (br s, 1H), 11.40 (br d, J=5.5 Hz, 1H), 8.34-8.20 (m, 1H), 8.02 (d, J=7.6 Hz, 1H), 7.52 (s, 1H), 7.42-7.36 (m, 2H), 7.34-7.21 (m, 7H), 6.89 (br d, J=8.8 Hz, 4H), 6.07-5.81 (m, 1H), 5.63 (br d, J=9.9 Hz, 1H), 4.89-4.60 (m, 4H), 4.56-4.35 (m, 1H), 4.01-3.90 (m, 1H), 3.89-3.81 (m, 1H), 3.75 (s, 7H), 3.69-3.52 (m, 5H), 3.46-3.38 (m, 2H), 3.46-3.38 (m, 1H), 3.19-3.10 (m, 4H), 2.98-2.90 (m, 2H), 2.87-2.74 (m, 4H), 2.25-2.14 (m, 1H), 1.24-1.10 (m, 18H), 1.04 (br d, J=6.6 Hz, 3H). ³¹P NMR (162 MHz, DMSO-d6) δ=150.15 (s, 1P), 149.85 (s, 1P).

Useful experimental procedures for certain L-DPSE-Dimer nucleotide amidites for, e.g., n012.

### Synthesis of WV-NU-184Rp-L-DPSE & WV-NU-184Sp-L-DPSE Amidites

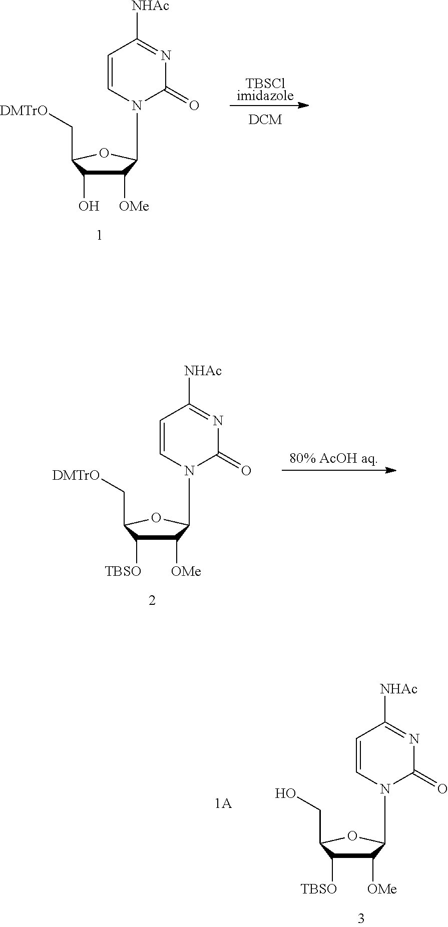

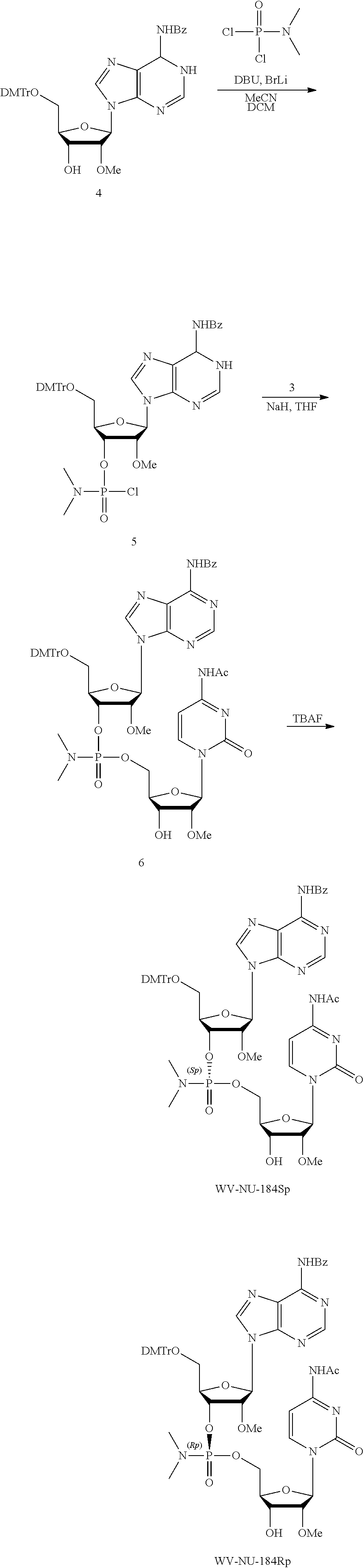

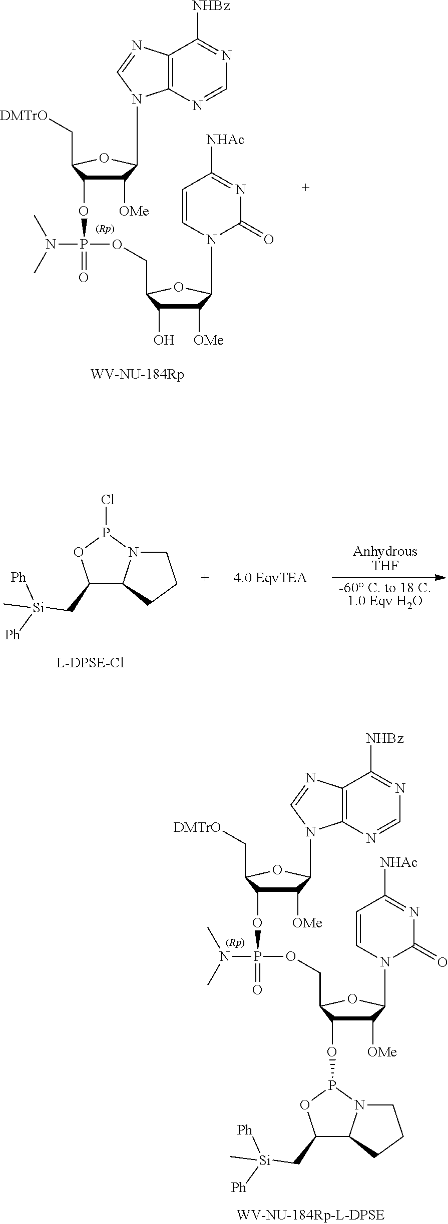

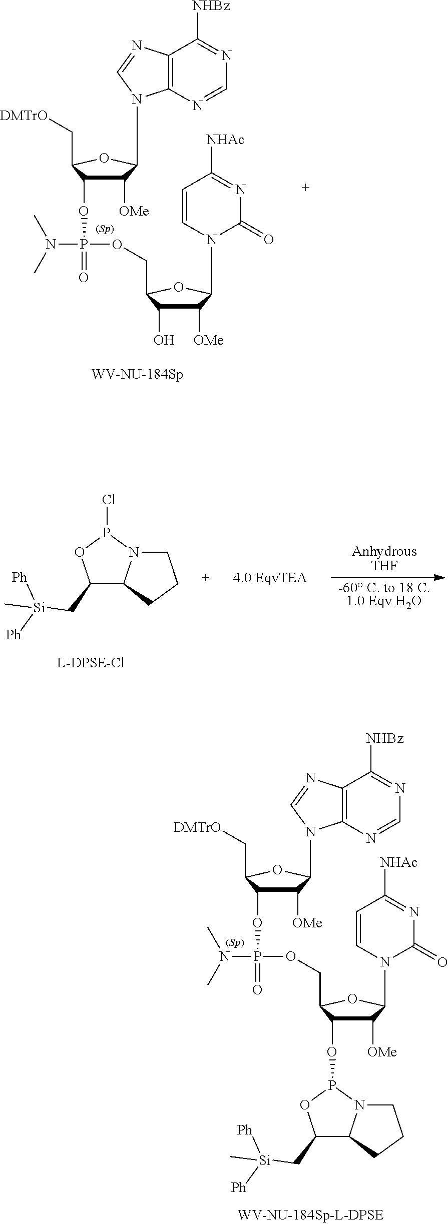

Step 1 G. Synthesis of compound 2.

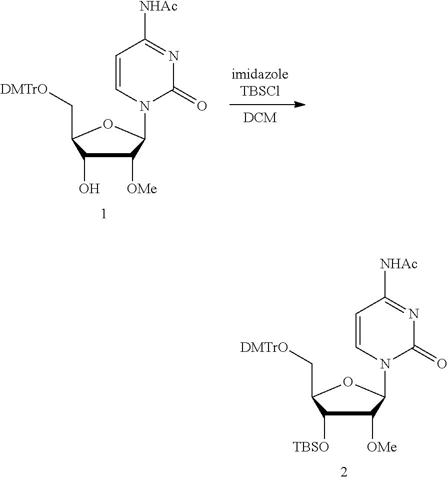

To a solution of compound 1 (27 g, 44.88 mmol, 1 eq.) and imidazole (9.17 g, 134.63 mmol, 3 eq.) in DCM (200 mL) was added to TBSC1 (13.53 g, 89.75 mmol, 11.00 mL, 2 eq.). The mixture was stirred at 25° C. for 12 hr. TLC (Ethyl acetate: Methanol=10:1, Rf=0.59) showed the product was detected. The reaction mixture was quenched by addition H₂O 300 mL, and then extracted with DCM 300 mL* 2, dried and concentrated under reduced pressure to give compound 2 (30 g, crude) as a yellow oil, which was used into the next step without further purification. TLC (Ethyl acetate: Methanol=10:1), Rf=0.59.

Step 2G. Synthesis of compound 3.

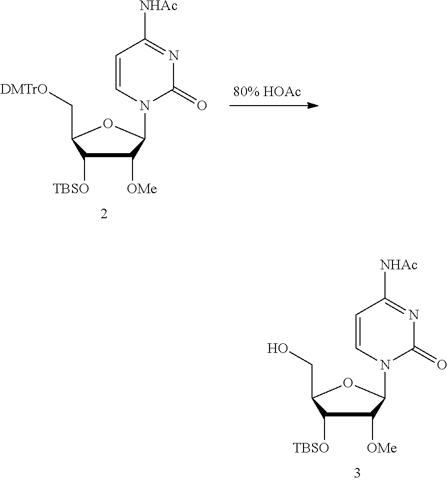

To a solution of compound 2 (35 g, 48.89 mmol, 1 eq.) in CH₃COOH (240 mL) and H₂O (60 mL), the mixture was stirred at 25° C. for 12 hr. The reaction mixture was quenched by addition NaHCO₃ adjust PH to 7 at 0° C., and then extracted with DCM 400 mL * 2. The combined organic layers were dried over and concentrated under reduced pressure to give a residue. The residue was purified by column chromatography (Ethyl acetate: Methanol=1:0 to 0:1) to get compound 3 (15 g, 36.27 mmol, 74.19% yield) as a white solid. LCMS: (M+H⁺) 414.54 . TLC (Ethyl acetate: Methanol=10:1), Rf=0.17.

Step 3G. Synthesis of compound 4A.

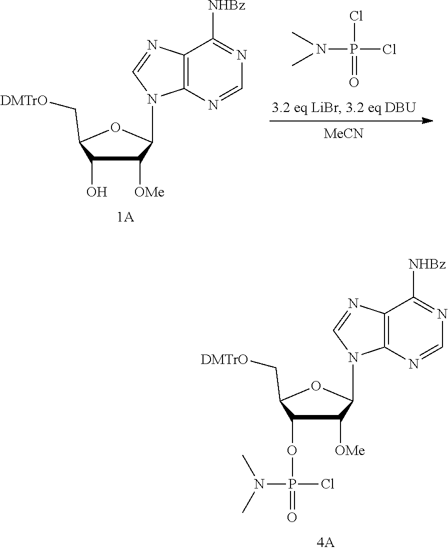

To a solution of compound 1A (20 g, 29.12 mmol, 1 eq.) in MeCN (500 mL) at N2 Protection , then in sequence was added BrLi (8.09 g, 93.19 mmol, 2.34 mL, 3.2 eq.) and DBU (14.19 g, 93.19 mmol, 14.05 mL, 3.2 eq.) at 0° C., N-dichlorophosphoryl-N-methyl-methanamine (7.07 g, 43.68 mmol, 1.5 eq.) was dropped, the mixture was stirred at 0° C. for 1 hr. LCMS showed Reactant 1 was consumed completely and desired m/z was detected. The reaction mixture was filtered and concentrated under reduced pressure at 15° C. to give a residue. The column was first alkalized with triethylamine: Petroleum ether=5:100 (1000 mL), then flush by petroleum ether (1000 mL). The residue was purified by column chromatography (SiO₂, Petroleum ether: Ethyl acetate=1:0 to 0:1). Compound 4A (11 g, 13.54 mmol, 46.50% yield) as a white solid. LCMS: (M−H⁺):811.2. TLC (Petroleum ether: Ethyl acetate=0:1), Rf=0.22.

Step 4G. Synthesis of compound 5.

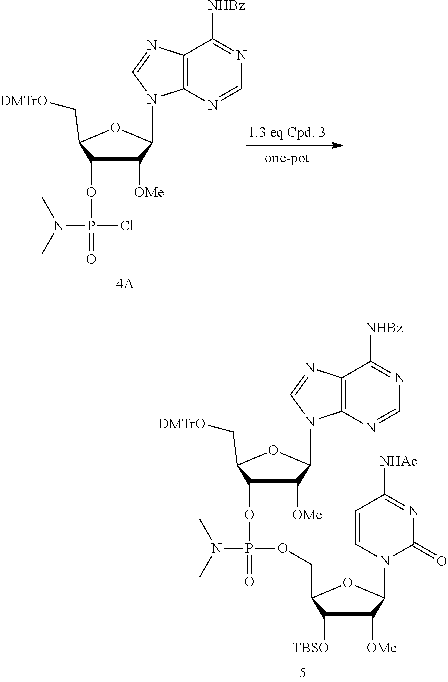

A solution of Cpd.3 (3.13 g, 7.58 mmol, 1 eq.) dissolved in THF (50 mL) at N₂ protection, then NaH (606.05 mg, 15.15 mmol, 60% purity, 2 eq.)was added to the solution at 0° C., the mixture was stirred at 0° C. for 5 min, and then Compound 3 (8 g, 9.85 mmol, 1.3 eq.) dissolved in THF (50 mL) was added, the mixture was stirred at 15° C. for 12 hr. The reaction mixture was diluted with H₂O 200 mL and extracted with DCM 200 mL * 2, dried over Na₂SO₄, filtered and concentrated under reduced pressure to give a residue. The crude product was purified by reversed-phase HPLC (column: Agela DuraShell C18 250*70 mm*10 um; mobile phase: [water (10 mM NH₄HCO₃)-ACN];B%: 65%-82%,@100 mL/min). Compound 5 (7 g, crude) was obtained as a white solid. LCMS: (M−H⁺):1188.5.

Step 5G. Synthesis of WV-NU-184Rp and WV-NU-184Sp.

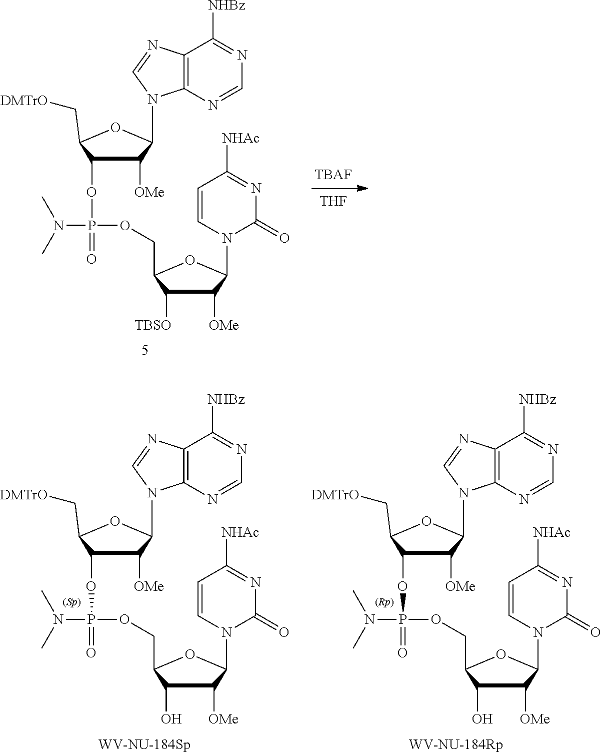

To a solution of Compound 5 (6 g, 5.04 mmol, 1 eq) in THF (60 mL) was added TBAF (1 M, 25.20 mL, 6 eq). The mixture was stirred at 15° C. for 12 hr. The reaction mixture was concentrated under reduced pressure to remove solvent. The residue was extracted with DCM 60 mL* 2. Use Methyl tert-butylether (100 mL) beating, filtered and then collect solids from filter cake. The crude product was purified by reversed-phase HPLC (column: Welch Xtimate C18 250*70 mm#10 um;mobile phase: [water(10 mM NH₄HCO3)-ACN];B%: 25%-65%, @100 mL/min). Compound WV-NU-184Rp (1.2 g, 1.08 mmol, 21.46% yield, 97% purity) and Compound WV-NU-184Sp (140 mg, 126.72 umol, 2.51% yield, 97.4% purity) was obtained as a white solid.

WV-NU-184Rp : ¹HNMR (400 MHz, DMSO-d₆) δ=11.27-11.20 (m, 1H), 10.94-10.90 (m, 1H), 8.67-8.60 (m, 2H), 8.13-8.08 (m, 1H), 8.04 (br d, J=7.3 Hz, 2H), 7.68-7.60 (m, 1H), 7.58-7.51 (m, 2H), 7.36 (br d, J=7.3 Hz, 2H), 7.28-7.15 (m, 8H), 6.83 (dd, J=2.8, 8.9 Hz, 4H), 6.22-6.16 (m, 1H), 5.87-5.83 (m, 1H), 5.37-5.32 (m, 1H), 5.19-5.11 (m, 1H), 4.97-4.88 (m, 1H), 4.37-4.31 (m, 1H), 4.31-4.24 (m, 1H), 4.21-4.13 (m, 1H), 4.11-3.98 (m, 3H), 3.76 (br dd, J=2.2, 4.6 Hz, 1H), 3.71 (s, 6H), 3.46 (s, 2H), 3.41 (s, 3H), 2.60 (s, 3H), 2.57 (s, 3H). LCMS: purity: 97.00%, M - H⁺=1074.3.

WV-NU-184Sp: LCMS: purity: 97.40%M - H⁺=1074.3. ¹H NMR (400 MHz, DMSO-d₆) 6=10.96-10.90 (m, 1H), 8.63 (s, 1H), 8.60 (s, 1H), 8.10 (d, J=7.5 Hz, 1H), 8.04 (br d, J=7.4 Hz, 2H), 7.68-7.62 (m, 1H), 7.59-7.52 (m, 2H), 7.36 (br d, J=7.4 Hz, 3H), 7.23 (br dd, J=4.1, 8.1 Hz, 8H), 6.82 (dd, J=4.3, 8.8 Hz, 5H), 6.19 (d, J=6.1 Hz, 1H), 5.87-5.83 (m, 1H), 5.37 (d, J=6.5 Hz, 1H), 5.10 (td, J=3.8, 7.5 Hz, 1H), 4.94 (br t, J=5.3 Hz, 1H), 4.46-4.41 (m, 1H), 4.24-4.09 (m, 3H), 4.09-3.98 (m, 3H), 3.77 (dd, J=2.4, 4.7 Hz, 2H), 3.71 (s, 7H), 3.62-3.56 (m, 1H), 3.47 (s, 4H), 3.40-3.36 (m, 5H), 2.65 (d, J=10.1 Hz, 9H), 2.35-2.31 (m, 1H), 2.08 (s, 3H), 1.78-1.73 (m, 1H).

Step 6G. Synthesis of WV-NU-184Rp-L-DPSE Amidite.

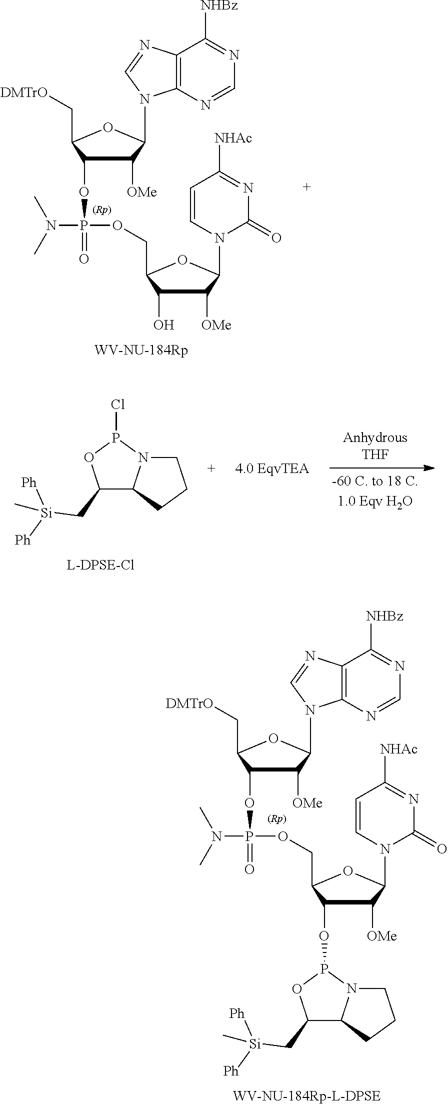

Compound WV-NU-184Rp (2.4 g, 2.23 mmol, 1.0 eq.) in a two necked flask (200 mL) was azeotroped three times with anhydrous toluene (30 mL) and was dried for 24 hrs on high vacuum. To the flask was added anhydrous THF (12 mL) under argon and solution was cooled to -60° C. to the reaction mixture was added triethyl amine (1.25 mL, 8.92 mmol, 4.0 eq.) followed by addition of DPSE-Cl (0.9 M) solution (5 mL, 4.4 mmol, 2.0 eq.) over the period of 5 min. The reaction mixture was warmed to room temperature and reaction progress was monitored by HPLC. After disappearance of starting material, reaction was quenched by addition of water and dried by addition of molecular sieve. The reaction mixture was filtered through fritted glass tube. Reaction flask and precipitate was washed with anhydrous THF (10 mL). Obtained filtrate was collected and solvent was removed under reduced pressure. The residue was purified by column chromatography (SiO₂, 50-100% Ethyl acetate (5% Et3N) in Hexanes) to give WV-NU-184Rp-L-DPSE Amidite (2.6 g, 82% yield) as a white solid. Chemical Formula: C₇₂H₈₀N₁₀O₁₅P₂Si, Cacl. Mass (M−H⁺):1414.52. LCMS: (M−H⁺): 1414.86. ¹H NMR (600 MHz, CDCl₃) δ=8.66 (s, 1H), 8.28 (d, J=7.5 Hz, 1H), 8.23 (s, 1H), 8.07-8.02 (m, 1H), 7.62 (td, J=7.2, 1.3 Hz, 1H), 7.57-7.50 (m, 4H), 7.45-7.41 (m, 1H), 7.41-7.36 (m, 1H), 7.38-7.34 (m, 1H), 7.36-7.30 (m, 4H), 7.32-7.26 (m, 1H), 7.26-7.21 (m, 1H), 6.85-6.79 (m, 3H), 6.21 (d, J=5.9 Hz, 1H), 5.90 (d, J=1.4 Hz, 1H), 5.15 (ddd, J=8.3, 4.9, 3.4 Hz, 1H), 4.94-4.86 (m, 1H), 4.46-4.37 (m, 1H), 4.30-4.17 (m, 2H), 4.14 (q, J=7.1 Hz, 1H), 3.79 (s, 5H), 3.61 (dd, J=10.6, 4.5 Hz, 1H), 3.55 (d, J=2.7 Hz, 4H), 3.42 (dd, J=10.6, 4.2 Hz, 1H), 3.37-3.32 (m, 1H), 3.18-3.12 (m, 1H), 2.67 (d, J=10.2 Hz, 4H), 2.25 (s, 2H), 1.81 (dt, J=8.1, 4.1 Hz, 1H), 1.72-1.63 (m, 1H), 1.45 (dd, J=14.6, 7.4 Hz, 1H), 1.36-1.30 (m, 1H), 1.27 (d, J=7.1 Hz, 1H), 1.25-1.18 (m, 1H), 1.05 (t, J=7.2 Hz, 1H), 0.65 (s, 3H). ¹³C NMR (151 MHz, CDCl₃) δ=171.16, 170.68, 164.64, 162.84, 158.67, 154.82, 152.52, 151.66, 149.57, 144.55, 144.22, 142.38, 136.33, 136.06, 135.43, 134.49, 134.40, 133.74, 132.76, 130.13, 130.10, 129.59, 129.52, 128.87, 128.28, 127.99, 127.96, 127.92, 127.89, 127.12, 123.81, 113.21, 96.42, 89.72, 86.97, 86.79, 83.25, 83.22, 82.98, 82.96, 81.04, 81.02, 80.91, 80.83, 78.97, 78.92, 73.11, 73.08, 68.71, 68.61, 67.43, 67.41, 63.84, 63.81, 62.70, 60.40, 58.87, 58.71, 55.26, 46.81, 46.58, 46.29, 36.72, 36.69, 26.96, 25.93, 25.91, 24.96, 21.07, 17.67, 17.65, 14.22, −3.41. ³¹P NMR (243 MHz, CDCl₃) δ=155.82, 10.35.

Step 7G. Synthesis of WV-NU-184Sp-L-DPSE Amidite.

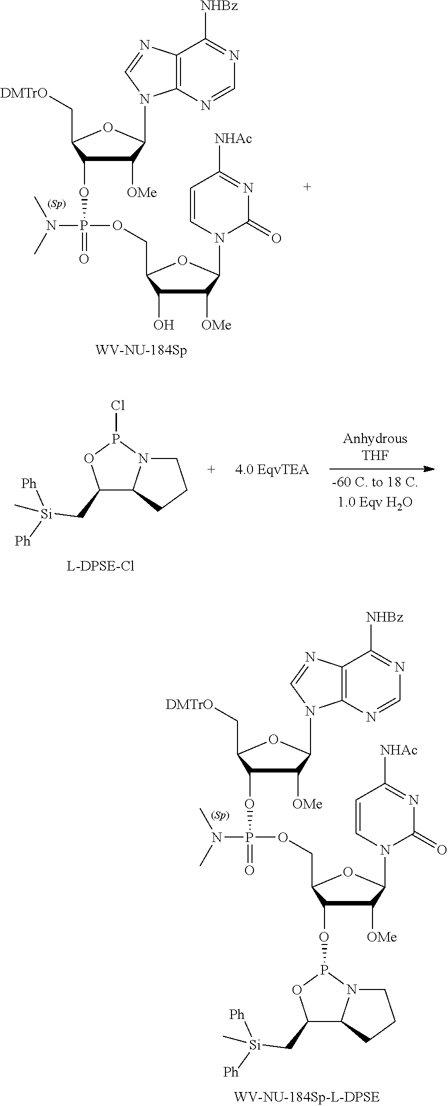

WV-NU-184Sp (510 mg) of compound was converted into WV-NU-184Sp-L-DPSE amidite under similar reaction conditions as WV-NU-184Rp into WV-NU-184Rp-L-DPSE amidite. Chemical Formula: C₇₂H₈₀N₁₀O₁₅P₂Si, Cacl. Mass (M−H⁺):1414.64. LCMS: (M−H⁺): 1414.83. ¹H NMR (600 MHz, CDCl₃) δ 8.91 (s, OH), 8.67 (s, 1H), 8.29 (d, J=2.2 Hz, 1H), 8.19 (d, J=7.5 Hz, 1H), 8.04 (d, J=7.6 Hz, 2H), 7.64 (t, J=7.6 Hz, 1H), 7.55 (dd, J=16.6, 7.4 Hz, 7H), 7.45 (d, J=7.7 Hz, 2H), 7.41-7.32 (m, 13H), 7.28 (d, J=1.9 Hz, 8H), 7.23 (t, J=7.4 Hz, 1H), 6.84-6.79 (m, 4H), 6.22 (d, J=6.7 Hz, 1H), 5.84 (s, 1H), 5.15-5.10 (m, 1H), 4.97 (d, J=6.0 Hz, 1H), 4.90 (q, J=7.0 Hz, 1H), 4.59 (t, J=3.4 Hz, 1H), 4.31 (dd, J=12.1, 4.9 Hz, 1H), 4.29-4.23 (m, 1H), 4.15 (t, J=8.4 Hz, 1H), 4.05 (d, J=11.9 Hz, 1H), 3.84-3.78 (m, 1H), 3.78 (s, 5H), 3.65-3.56 (m, 4H), 3.56-3.48 (m, 5H), 3.37 (t, J=7.4 Hz, 1H), 3.31 (t, J=7.2 Hz, 1H), 3.17 (d, J=11.3 Hz, 1H), 2.75 (dd, J=10.2, 2.2 Hz, 5H), 2.55 (d, J=7.4 Hz, 1H), 2.20 (d, J=2.3 Hz, 3H), 2.07 (t, J=1.8 Hz, 1H), 1.99 (d, J=2.0 Hz, 1H), 1.83 (d, J=10.8 Hz, 1H), 1.68 (dd, J=14.3, 7.5 Hz, 2H), 1.47 (dd, J=14.6, 7.5 Hz, 1H), 1.35 (dd, J=13.7, 7.8 Hz, 1H), 1.31-1.19 (m, 2H), 1.19-1.14 (m, 1H), 1.05 (t, J=7.4 Hz, 1H), 0.71-0.64 (m, 3H). ¹³C NMR (151 MHz, CDCl₃) δ −3.42, −3.28, 14.22, 14.88, 17.73, 17.75, 23.39, 25.02, 25.91, 25.94, 26.99, 34.49, 36.79, 36.81, 46.55, 46.79, 55.23, 55.26, 58.76, 60.41, 62.85, 63.47, 63.50, 67.43, 67.44, 68.59, 68.67, 74.04, 74.07, 76.82, 77.03, 77.25, 78.99, 79.05, 80.71, 80.93, 82.80, 83.63, 83.65, 86.41, 86.87, 90.20, 96.20, 113.20, 123.69, 127.06, 127.86, 127.91, 127.97, 128.00, 128.10, 128.14, 128.32, 128.90, 129.51, 129.57, 130.12, 130.18, 132.80, 133.73, 134.41, 134.50, 134.56, 135.45, 135.49, 136.07, 136.37, 142.60, 144.31, 144.54, 149.51, 151.79, 154.82, 158.60, 158.62, 162.46. ³¹P NMR (243 MHz, CDCl₃) δ=155.38, 11.27.

### Synthesis of WV-NU-185 Sp-L-DPSE & WV-NU-185Rp-L-DPSE Amidites

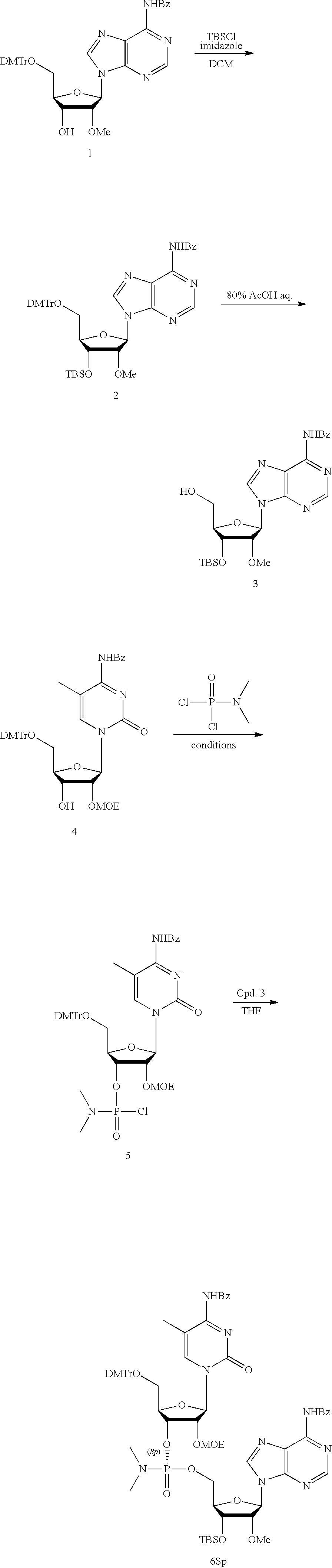

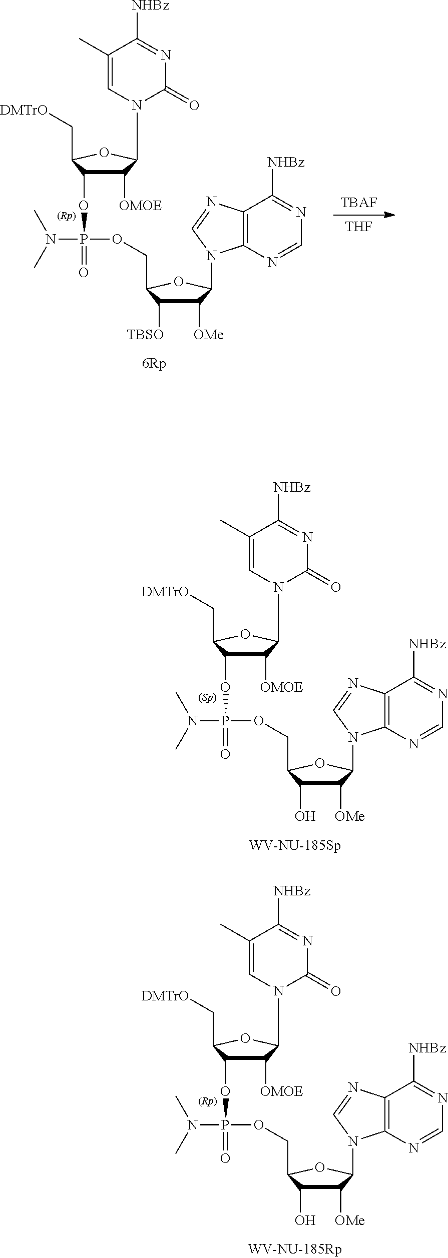

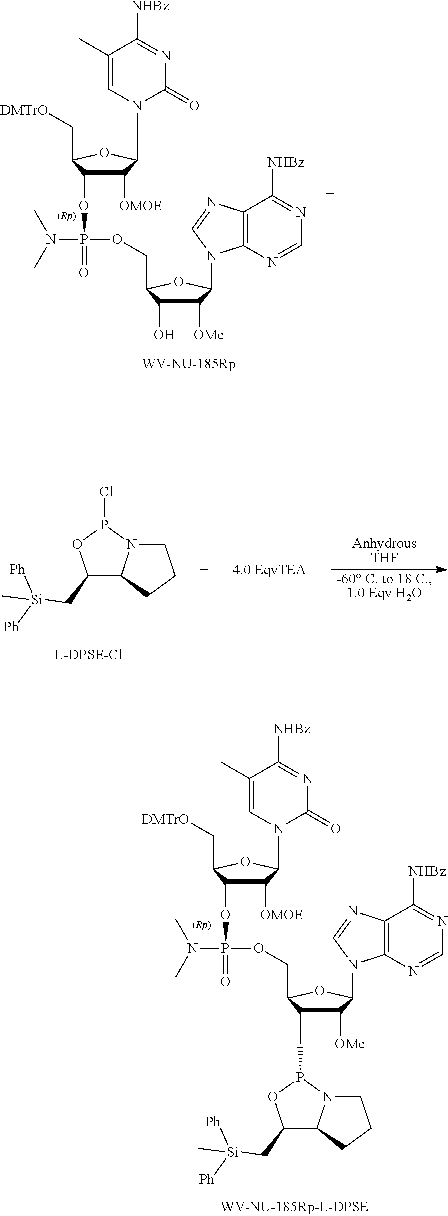

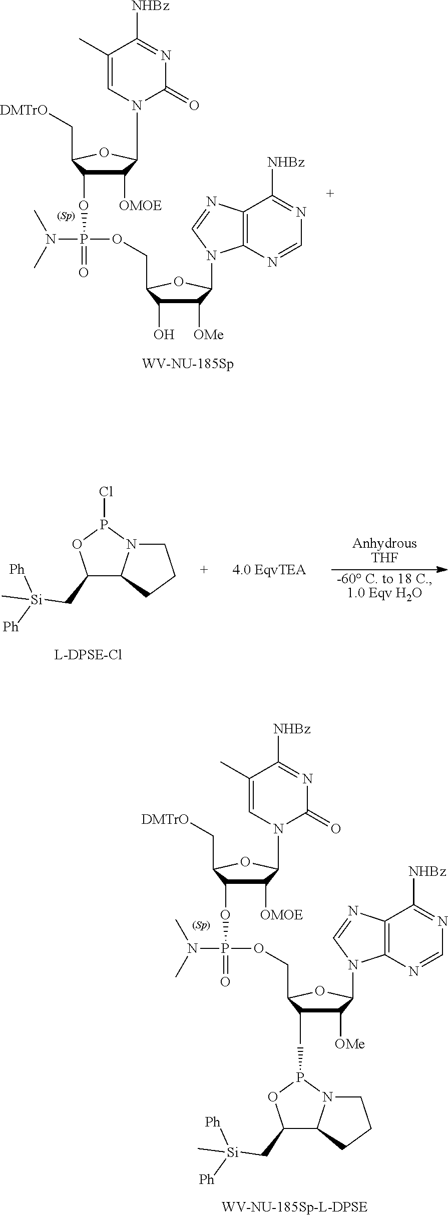

Step 1F. Synthesis of compound 2.

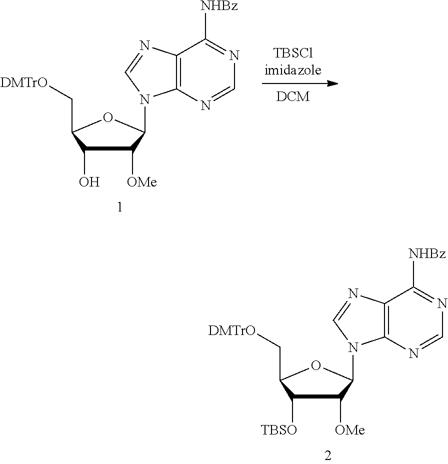

To a solution of compound 1 (35 g, 50.89 mmol, 1 eq.) in DCM (400 mL) was added imidazole (10.39 g, 152.67 mmol, 3 eq.) and TBSC1 (15.34 g, 101.78 mmol, 12.47 mL, 2 eq.). The mixture was stirred at 25° C. for 12 hr. LCMS showed compound 1 was consumed completely and desired mass was detected. The reaction mixture was washed by addition water 200 mL, and then extracted with DCM (200 mL * 3). The combined organic layers were dried over Na₂SO₄ filtered and concentrated under reduced pressure to give a residue. Compound 2 (40.8 g, crude) was obtained as a colorless oil. LCMS (M+H+): 801.3.

Step 2F. Synthesis of compound 3.

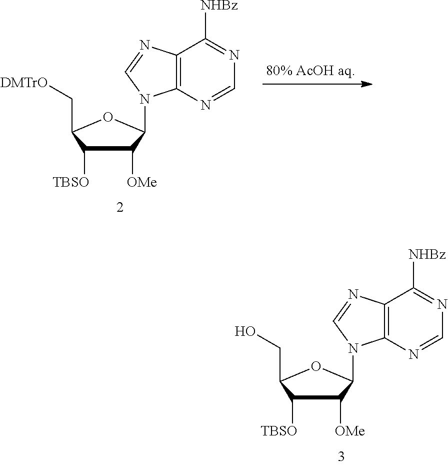

To a solution of compound 2 (40.8 g, 50.87 mmol, 1 eq.) in the mixture of AcOH (240 mL) and H₂O (60 mL), the mixture was stirred at 25° C. for 12 hr. TLC: Petroleum ether: Ethyl acetate=0:1, Rf=0.47. The reaction mixture was quenched by addition NaHCO3 adjust PH=7 at 0° C., and then extracted with DCM (200 mL *4). The combined organic layers were dried over and concentrated under reduced pressure to give a residue. The residue was purified by column chromatography (SiO₂, Petroleum ether: Ethyl acetate=30:1 to 0:1, 5% TEA), after purification Compound 3 (22 g, 44.03 mmol, 86.55% yield) was obtained as a white solid. LCMS (H−M+): 498.2, purity: 89.4%. TLC: Petroleum ether: Ethyl acetate=0:1, Rf=0.47.

Step 3F. Synthesis of compound 4A.

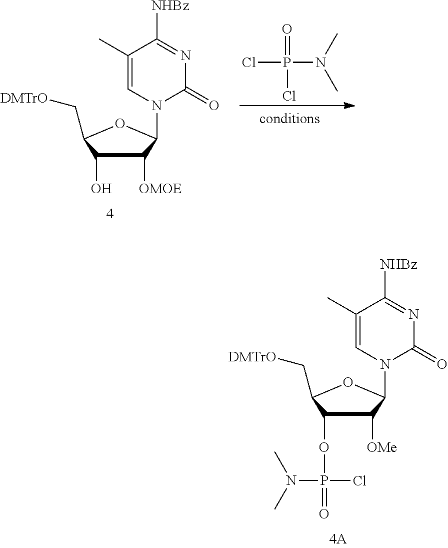

To a solution of compound 4 (6 g, 8.31 mmol, 1 eq.) in DCM (8 mL) and MeCN (24 mL) was added LiBr (2.31 g, 26.60 mmol, 667.70 uL, 3.2 eq.) at 0° C., and then DBU (4.05 g, 26.60 mmol, 4.01 mL, 3.2 eq.) was added, N-dichlorophosphoryl-N-methyl-methanamine (2.15 g, 13.30 mmol, 1.6 eq.) was dropped in N2, the mixture was stirred at 0° C. for 2 hr. TLC (Petroleum ether: Ethyl acetate=0:1, Rf=0.75) indicated compound 4 consumed completely and new spot formed. The reaction mixture was filtered and concentrated under reduced pressure at 20° C. to give a residue. The residue was purified by column chromatography (SiO₂, Petroleum ether: Ethyl acetate=0:1 to 1:1, 5% TEA). Compound 4 A (5 g, 5.90 mmol, 70.99% yield) was obtained as a white oil. TLC: Petroleum ether: Ethyl acetate=0:1, Rf=0.75.

Step 4F. Synthesis of compound WV-NU-185Sp and WV-NU-185Rp.

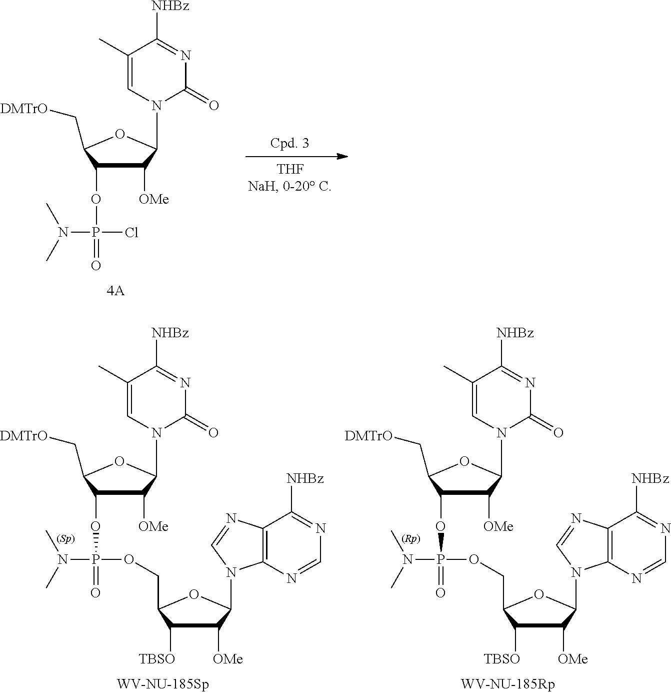

To a solution of compound 3 (2.90 g, 5.81 mmol, 1 eq.) in THF (50 mL) was added NaH (697.13 mg, 17.43 mmol, 60% purity, 3 eq.) at 0° C. for 0.5 hr, and then added compound 4A (5 g, 5.81 mmol, 1 eq.) in N2. The mixture was stirred at 0-20° C. for 2 hr. The reaction mixture was quenched by addition NH₄Cl30 mL at 0° C., and extracted with DCM (50 mL *3). The combined organic layers were dried over Na₂SO₄ filtered and concentrated under reduced pressure to give a residue. The crude product was purified by reversed-phase HPLC (column: C18 20-35 um 100A 100 g; mobile phase: [water (10 mM NH₄HCO3)-ACN]; B%: 70%-95%, 20min, after purification Compound 5Sp (1.4 g, 1.07 mmol, 18.39% yield) was obtained as a white solid. Compound 5Rp (2.1 g, 1.60 mmol, 27.58% yield) was obtained as a white solid. ¹HNMR (400 MHz, DMSO-d₆) δ=12.89 (s, 1H), 11.22 (s, 1H), 8.75 (s, 1H), 8.69 (s, 1H), 8.16 (br d, J=7.4 Hz, 2H), 8.04 (d, J=7.3 Hz, 2H), 7.77 (s, 1H), 7.68-7.46 (m, 6H), 7.40 (br d, J=7.5 Hz, 2H), 7.33-7.18 (m, 7H), 6.88 (dd, J=5.3, 8.8 Hz, 4H), 6.15 (d, J=4.9 Hz, 1H), 5.88 (br d, J=4.4 Hz, 1H), 4.93-4.85 (m, 1H), 4.68-4.61 (m, 2H), 4.38 (br t, J=4.5 Hz, 1H), 4.28 (br d, J=4.1 Hz, 1H), 4.10 (br s, 3H), 3.85-3.78 (m, 1H), 3.71 (d, J=1.9 Hz, 7H), 3.46 (br s, 2H), 3.40-3.27 (m, 11H), 3.18 (s, 3H), 2.07 (s, 1H), 1.53 (s, 2H), 0.89 (s, 9H), 0.15-0.06 (m, 6H). LCMS (M−H⁺): 1309.4; purity: 98.63%. ¹HNMR (400 MHz, DMSO-d₆) δ=12.91 (br s, 1H), 11.22 (s, 1H), 8.75 (s, 1H), 8.65 (s, 1H), 8.16 (br d, J=7.5 Hz, 2H), 8.03 (d, J=7.3 Hz, 2H), 7.81 (s, 1H), 7.67-7.45 (m, 6H), 7.41-7.36 (m, 2H), 7.32 (t, J=7.6 Hz, 2H), 7.24 (dd, J=4.1, 8.8 Hz, 5H), 6.89 (dd, J=2.5, 9.0 Hz, 4H), 6.17 (d, J=5.0 Hz, 1H), 5.89 (br d, J=3.5 Hz, 1H), 5.91-5.86 (m, 1H), 4.85-4.77 (m, 1H), 4.65 (br d, J=3.9 Hz, 1H), 4.62-4.56 (m, 1H), 4.38-4.33 (m, 1H), 4.27-4.20 (m, 2H), 4.17-4.12 (m, 2H), 3.72 (s, 6H), 3.41 (br t, J=4.4 Hz, 2H), 3.35 (s, 3H), 3.17 (d, J=5.3 Hz, 7H), 3.11 (s, 3H), 2.40 (br d, J=10.3 Hz, 6H), 1.58 (s, 2H), 0.91 (s, 9H), 0.12 (d, J=6.3 Hz, 6H). LCMS (M−H⁺): 1309.4; purity: 97.74%.

Step 5F. Synthesis of compound WV-NU185Sp: To a solution of compound 5S (1.40 g, 1.07 mmol, 1 eq.) in THF (14 mL) was added TBAF (1 M, 3.20 mL, 3 eq.). The mixture was stirred at 25° C. for 2 hr. TLC: (Dichloromethane: Methanol=8:1, Rf=0.49) showed the compound 5S consumed completely and one new one spot formed. The reaction mixture was and then diluted with water 20 mL and extracted with DCM 60 mL (20 mL * 3). The combined organic layers were dried over Na₂SO₄ filtered and concentrated under reduced pressure to give a residue. The crude product was triturated with Methyl tert-butyl ether (200 ml at 25 oC for 30 min. And filter to white solid. The crude product was purified by reversed-phase HPLC (Phenomenex Titank C18 Bulk 250*70 mm 10u; mobile phase: [water (10 mM NH₄HCO3)-ACN]; B%: 53%-83%, 20min. Compound WV-NU-185Sp (0.9 g, 752.38 umol, 75.84% yield) was obtained as a white solid. ¹HNMR (400 MHz, DMSO-d₆) δ=8.75 (s, 1H), 8.66 (s, 1H), 8.15 (br s, 1H), 8.04 (br d, J=7.5 Hz, 2H), 7.80 (br s, 1H), 7.67-7.46 (m, 7H), 7.41 (br d, J=7.8 Hz, 2H), 7.33-7.25 (m, 6H), 7.25-7.19 (m, 1H), 6.93-6.83 (m, 4H), 6.16 (d, J=5.3 Hz, 1H), 5.88 (br d, J=4.1 Hz, 1H), 5.54 (d, J=5.6 Hz, 1H), 4.92-4.86 (m, 1H), 4.52 (br t, J=5.1 Hz, 1H), 4.42 (br s, 1H), 4.36 (br s, 1H), 4.31-4.26 (m, 1H), 4.10 (br s, 3H), 3.81 (br s, 2H), 3.71 (s, 6H), 3.46 (br s, 2H), 3.36 (d, J=9.8 Hz, 5H), 3.18 (s, 3H), 2.54 (s, 3H), 2.52-2.50 (m, 6H), 1.52 (br s, 2H). ¹³C NMR (151 MHz, CDCl₃) δ −3.32, 12.57, 14.22, 17.75, 17.78, 25.96, 25.98, 27.25, 36.68, 36.70, 46.56, 46.79, 55.27, 58.73, 59.05, 60.40, 62.50, 64.54, 64.57, 67.88, 67.90, 70.06, 70.13, 70.41, 72.48, 73.74, 73.78, 76.87, 77.08, 77.29, 79.30, 79.36, 81.33, 81.36, 81.81, 81.83, 82.06, 82.11, 82.72, 82.76, 86.69, 87.33, 87.77, 112.28, 113.36, 123.79, 127.29, 127.90, 127.93, 128.00, 128.03, 128.09, 128.12, 128.35, 128.87, 129.41, 129.43, 129.92, 130.25, 132.45, 132.78, 133.75, 134.35, 134.43, 134.51, 134.61, 135.03, 135.20, 135.91, 136.58, 136.80, 137.17, 142.27, 144.10, 148.19, 149.72, 151.25, 152.52, 158.79, 158.83, 159.61, 164.65, 171.15, 179.56. LCMS (M−H⁺):1194.4, purity: 97.70%. TLC: Dichloromethane: Methanol=8:1, Rf=0.49.

Step 6F. Synthesis of compound WV-NU-185Rp: To a solution of compound 5R (2.1 g, 1.60 mmol, 1 eq.) in THF (20 mL) was added TBAF (1 M, 4.81 mL, 3 eq.). The mixture was stirred at 25° C. for 2 hr. The reaction mixture was filtered and concentrated under reduced pressure to give a residue. The reaction mixture was and then diluted with water 20 mL and extracted with Ethyl acetate 60 mL (20 mL * 3). The combined organic layers were dried over Na2SO4 filtered and concentrated under reduced pressure to give a residue. The crude product was triturated with Methyl tert-butyl ether (100 ml at 25 oC for 30min. Filtered the cake and then under reduced pressure to get a white solid. The crude product was purified by reversed-phase HPLC (column: Welch Xtimate C18 250*70 mm#10 um; mobile phase: [water (10 mM NH4HCO3)-ACN]; B%: 55%-90%, 20min. Compound WV-NU-185Rp (1.17 g, 978.10 umol, 41.35% yield) was obtained as a white solid. ¹H NMR (400 MHz, DMSO-d6) δ=8.75 (s, 1H), 8.64 (s, 1H), 8.12 (br d, J=7.3 Hz, 2H), 8.03 (br d, J=7.4 Hz, 2H), 7.83 (br s, 1H), 7.67-7.46 (m, 7H), 7.40-7.29 (m, 5H), 7.24 (br dd, J=2.8, 8.7 Hz, 5H), 6.90 (dd, J=1.8, 8.7 Hz, 4H), 6.18 (d, J=4.9 Hz, 1H), 5.90 (d, J=3.8 Hz, 1H), 5.54 (d, J=5.4 Hz, 1H), 4.87-4.80 (m, 1H), 4.48-4.40 (m, 2H), 4.36 (br t, J=4.4 Hz, 1H), 4.28-4.20 (m, 2H), 4.16 (br d, J=4.1 Hz, 2H), 3.82-3.75 (m, 2H), 3.72 (s, 6H), 3.43 (br t, J=4.4 Hz, 2H), 3.36 (d, J=11.9 Hz, 6H), 3.14 (s, 3H), 2.42 (d, J=10.3 Hz, 6H), 1.55 (s, 3H). LCMS (M+H⁺):1194.3, purity: 97.18%.

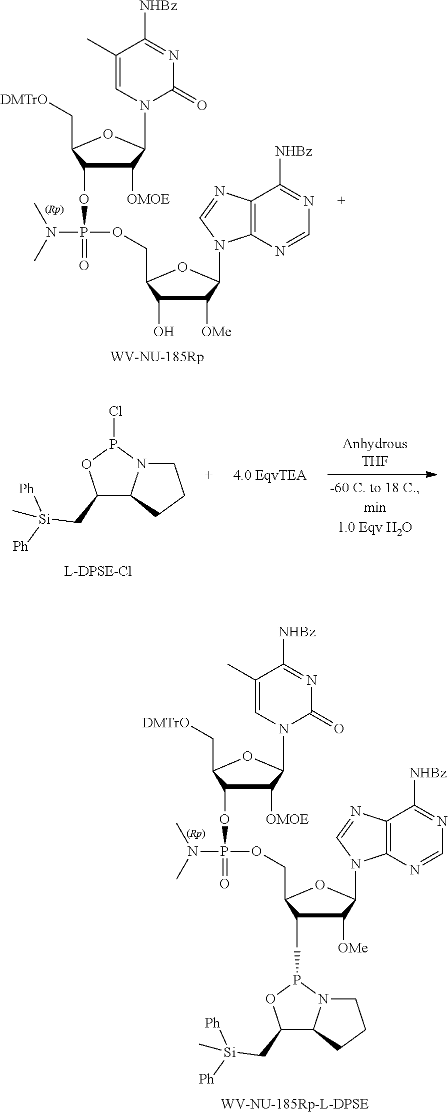

Step 6F. Synthesis WV-NU-185Rp-L-DPSE amidites: Compound WV-NU-185Rp (2.3 g, 2.03 mmol, 1.0 eq.) in a two necked flask (200 mL) was azeotroped three times with anhydrous toluene (30 mL) and was dried for 24 hrs on high vacuum. To the flask was added anhydrous THF (12 mL) under argon and solution was cooled to -60° C. to the reaction mixture was added triethyl amine (1.15 mL, 8.21 mmol, 4.0 eq.) followed by addition of DPSE-Cl (0.9 M) solution (4.56 mL, 4.1 mmol, 2.0 eq.) over the period of 5 min. The reaction mixture was warmed to room temperature and reaction progress was monitored by HPLC. After disappearance of starting material, reaction was quenched by addition of water and dried by addition of molecular sieve. The reaction mixture was filtered through fritted glass tube. Reaction flask and precipitate was washed with anhydrous THF (10 mL). Obtained filtrate was collected and solvent was removed under reduced pressure. The residue was purified by column chromatography (SiO_{2, 50}-100% Ethyl acetate (5% Et₃N) in Hexanes) to give WV-NU-185Rp-L-DPSE Amidite (2.4 g, 80% yield) as a white solid. Chemical Formula: C₈₁H₉₀N₁₀O₁₅P₂Si, Cacl. Mass (M−H⁺):1532.70. LCMS: (M−H⁺): 1532.81. ¹H NMR (600 MHz, CDCl₃) δ 13.07 (s, 1H), 9.01 (s, 1H), 8.67 (s, 1H), 8.24-8.19 (m, 2H), 8.01 (s, 1H), 7.97-7.93 (m, 2H), 7.73 (d, J=1.4 Hz, 1H), 7.51 (td, J=7.2, 1.3 Hz, 1H), 7.46 (d, J=1.4 Hz, 1H), 7.46-7.39 (m, 6H), 7.38-7.31 (m, 3H), 7.31-7.19 (m, 10H), 7.21-7.12 (m, 3H), 6.79-6.72 (m, 3H), 6.15 (d, J=5.6 Hz, 1H), 5.86 (d, J=4.2 Hz, 1H), 4.92 (dt, J=8.4, 4.3 Hz, 1H), 4.87 (dt, J=8.6, 5.9 Hz, 1H), 4.72 (dt, J=9.7, 5.0 Hz, 1H), 4.36-4.29 (m, 2H), 4.20 (t, J=4.6 Hz, 1H), 4.08-3.98 (m, 2H), 3.91-3.79 (m, 2H), 3.75-3.67 (m, 6H), 3.53-3.43 (m, 3H), 3.41 (ddd, J=15.5, 12.3, 6.5 Hz, 1H), 3.34 (dd, J=11.0, 2.2 Hz, 1H), 3.31 (s, 2H), 3.20 (s, 2H), 3.11 (tdd, J=10.3, 8.6, 4.2 Hz, 1H), 2.58 (d, J=10.4 Hz, 4H), 1.79 (ddt, J=12.8, 8.9, 5.1 Hz, 1H), 1.54 (dd, J=14.6, 8.6 Hz, 1H), 1.41-1.32 (m, 4H), 1.24-1.14 (m, 1H), 0.57 (s, 3H). ¹³C NMR (151 MHz, CDCl₃) δ −3.35, −3.25, 12.44, 14.22, 17.70, 17.73, 21.07, 25.95, 25.97, 27.12, 36.53, 36.55, 46.61, 46.85, 55.30, 58.62, 58.63, 58.85, 60.41, 61.88, 65.23, 65.27, 67.75, 67.77, 70.27, 70.33, 70.41, 72.05, 72.08, 72.30, 76.84, 77.05, 77.27, 79.06, 79.12, 81.48, 81.79, 81.84, 82.23, 82.25, 82.41, 82.45, 82.47, 87.18, 87.20, 87.80, 112.18, 113.29, 113.32, 123.61, 127.43, 127.88, 127.94, 127.95, 128.06, 128.11, 128.51, 128.88, 129.45, 129.48, 129.93, 130.32, 130.35, 132.43, 132.74, 133.80, 134.37, 134.40, 134.51, 134.54, 134.98, 135.11, 135.99, 136.50, 136.66, 137.18, 142.17, 143.90, 147.99, 149.60, 151.51, 152.62, 158.90, 158.91, 159.67, 164.65, 171.15, 179.60. ³¹P NMR (243 MHz, CDCl₃) δ=153.87, 11.35.

Step 7F. Synthesis of WV-NU-185Sp-L-DPSE amidite.

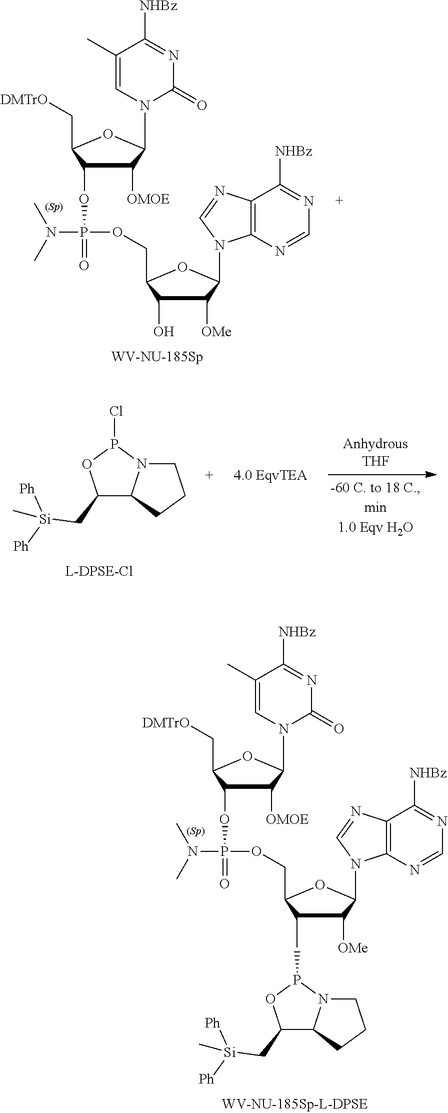

WV-NU-185Sp (750 mg) of compound was converted into WV-NU-185Sp-L-DPSE amidite same as WV-NU-185Rp into WV-NU-185Rp-L-DPSE amidite (680 mg, 70% yield). Chemical Formula: C₈₁H₉₀N₁₀O₁₅P₂Si, Cacl. Mass (M−H⁺):1532.70. LCMS: (M−H⁺): 1532.75. ^{1H} NMR (600 MHz, CDCl₃) δ=13.17 (s, 1H), 9.08 (s, 1H), 8.77 (s, 1H), 8.33-8.29 (m, 2H), 8.11 (s, 1H), 8.05 (d, J=7.6 Hz, 2H), 7.82 (d, J=1.5 Hz, 1H), 7.64-7.58 (m, 1H), 7.58-7.48 (m, 7H), 7.45 (dd, J=15.4, 7.7 Hz, 4H), 7.41-7.29 (m, 9H), 7.26 (td, J=7.4, 5.6 Hz, 3H), 6.88-6.82 (m, 4H), 6.24 (d, J=5.6 Hz, 1H), 5.95 (d, J=4.1 Hz, 1H), 5.02 (dt, J=8.3, 4.3 Hz, 1H), 4.97 (dt, J=8.6, 5.9 Hz, 1H), 4.82 (dt, J=9.6, 5.0 Hz, 1H), 4.46-4.39 (m, 2H), 4.30 (t, J=4.6 Hz, 1H), 4.17-4.07 (m, 2H), 4.00-3.88 (m, 2H), 3.82 (ddd, J=11.4, 5.0, 3.4 Hz, 1H), 3.79 (d, J=4.3 Hz, 6H), 3.63-3.55 (m, 3H), 3.57-3.47 (m, 2H), 3.44 (dd, J=11.0, 2.2 Hz, 1H), 3.41 (s, 3H), 3.30 (s, 3H), 3.25-3.16 (m, 1H), 2.67 (d, J=10.4 Hz, 5H), 1.88 (ddt, J=12.2, 8.0, 3.8 Hz, 1H), 1.79-1.70 (m, 1H), 1.63 (dd, J=14.7, 8.6 Hz, 1H), 1.50-1.42 (m, 1H), 1.34-1.24 (m, 2H), 0.67 (s, 3H). ^{31P} NMR (243 MHz, CDCl₃) δ 153.79, 11.10. ¹³C NMR (151 MHz, CDCl₃) δ −3.32, 12.57, 14.22, 17.75, 17.78, 25.96, 25.98, 27.25, 36.68, 36.70, 46.56, 46.79, 55.27, 58.73, 59.05, 60.40, 62.50, 64.54, 64.57, 67.88, 67.90, 70.06, 70.13, 70.41, 72.48, 73.74, 73.78, 76.87, 77.08, 77.29, 79.30, 79.36, 81.33, 81.36, 81.81, 81.83, 82.06, 82.11, 82.72, 82.76, 86.69, 87.33, 87.77, 112.28, 113.36, 123.79, 127.29, 127.90, 127.93, 128.00, 128.03, 128.09, 128.12, 128.35, 128.87, 129.41, 129.43, 129.92, 130.25, 132.45, 132.78, 133.75, 134.35, 134.43, 134.51, 134.61, 135.03, 135.20, 135.91, 136.58, 136.80, 137.17, 142.27, 144.10, 148.19, 149.72, 151.25, 152.52, 158.79, 158.83, 159.61, 164.65, 171.15, 179.56.

### Synthesis of WV-NU-186Sp-L-DPSE & WV-NU-186Rp-L-DPSE Amidites.

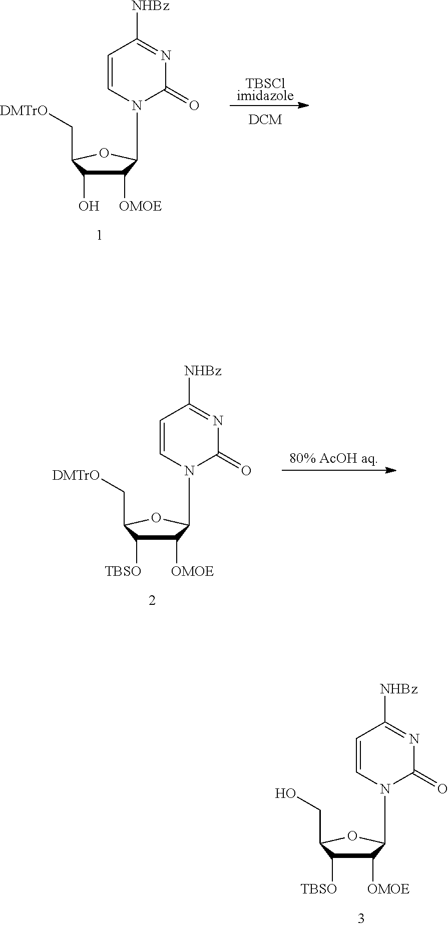

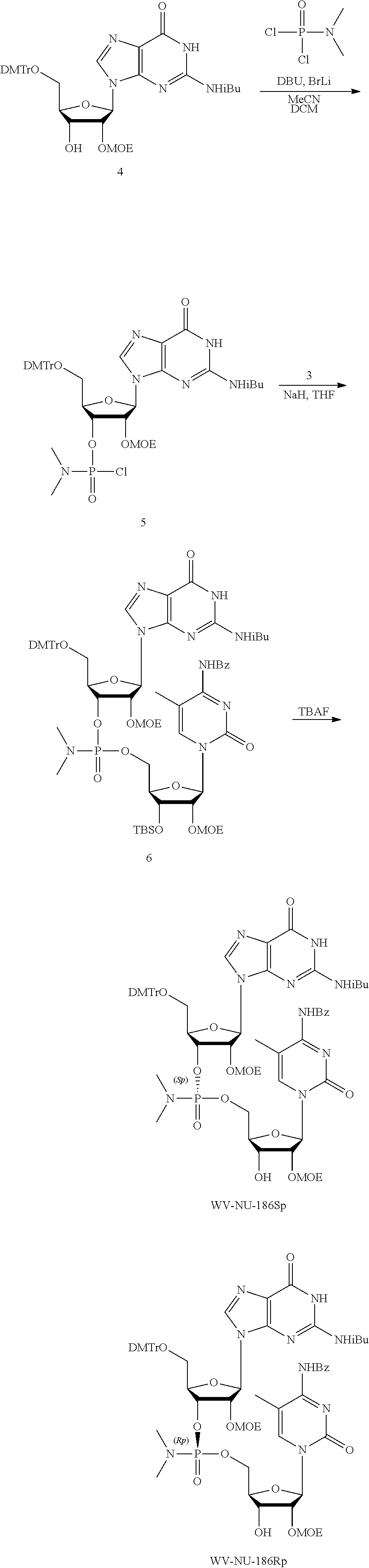

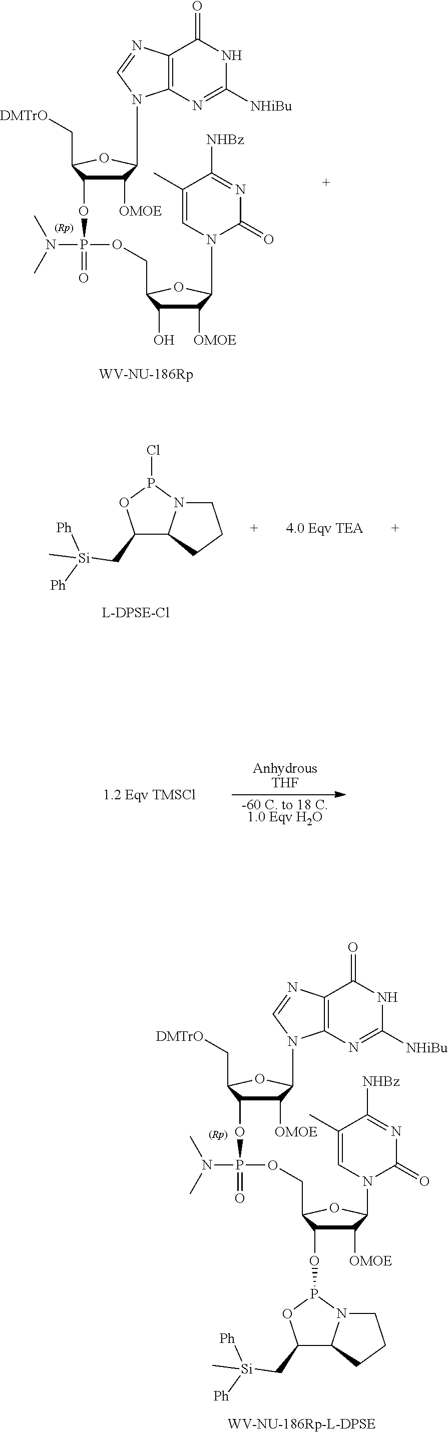

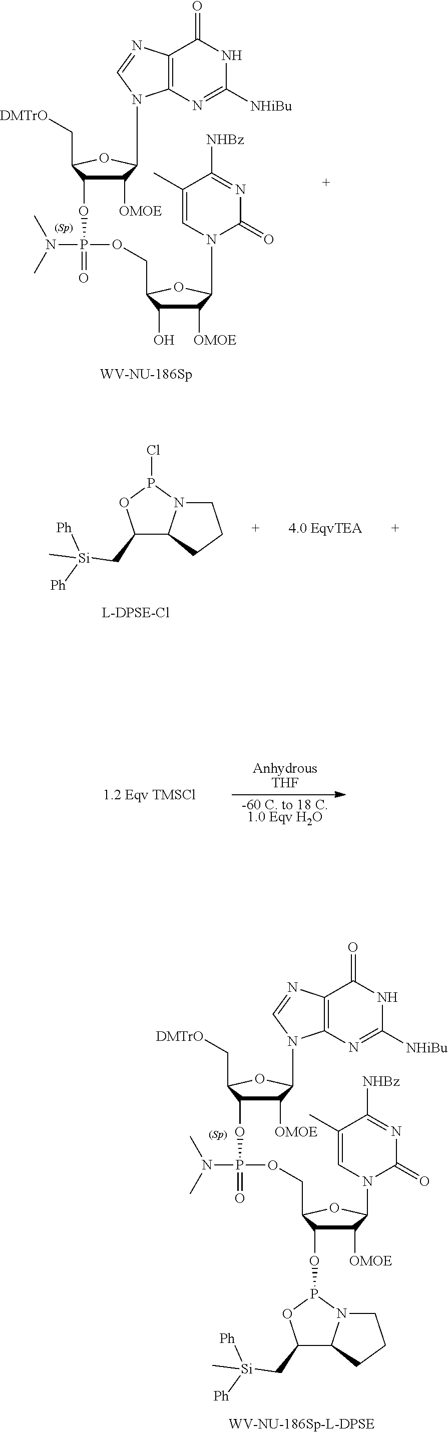

Step 1H. Preparation of compound 2.

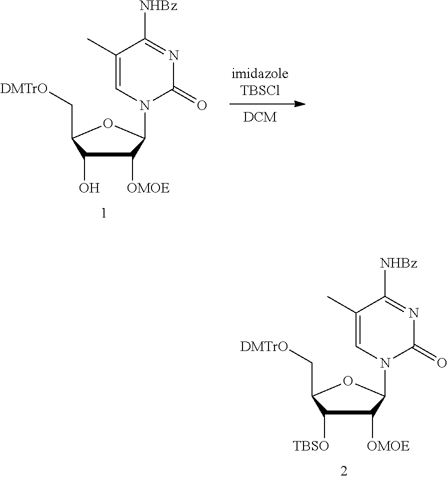

To a solution of compound 1 (32 g, 44.33 mmol, 1 eq.) in DCM (360 mL) was added imidazole (12.07 g, 177.34 mmol, 4 eq.) and TBSC1 (20.05 g, 133.00 mmol, 16.30 mL, 3 eq.). The mixture was stirred at 20° C. for 12 hr. The reaction mixture was diluted with water 360 mL and extracted with DCM (360 mL * 3). The combined organic layers were dried over Na₂SO₄ filtered and concentrated under reduced pressure to give a residue. Compound 2 (30 g, crude) was obtained as a yellow oil was used into the next step without further purification. LCMS: (M−H⁺) 835.05.

Step 2H. Preparation of compound 3.

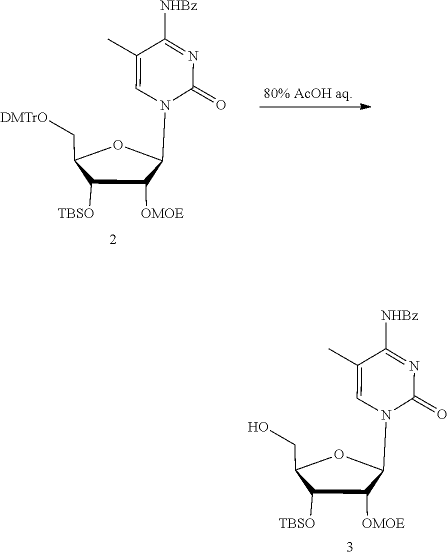

To a solution of compound 2 (30 g, 35.88 mmol, 1 eq.) in CH₃COOH (320 mL) and H₂O (80 mL), the mixture was stirred at 15° C. for 12 hr. TLC (Petroleum ether/Ethyl acetate=0/1) indicated compound 2 was consumed completely and two new spots formed. The reaction mixture was quenched by addition NaHCO3 adjust pH to 7 at 0° C., and then extracted with DCM 400 mL * 2. The combined organic layers were dried over and concentrated under reduced pressure to give a residue. The residue was purified by column chromatography (SiO₂, Petroleum ether/Ethyl acetate=1/0 to 0/1). Compound 3 (17.5 g, 32.79 mmol, 91.38% yield) was obtained as a white solid. TLC (Petroleum ether: Ethyl acetate=0:1), Rf=0.14.

Step 3H. Preparation of compound 4A.

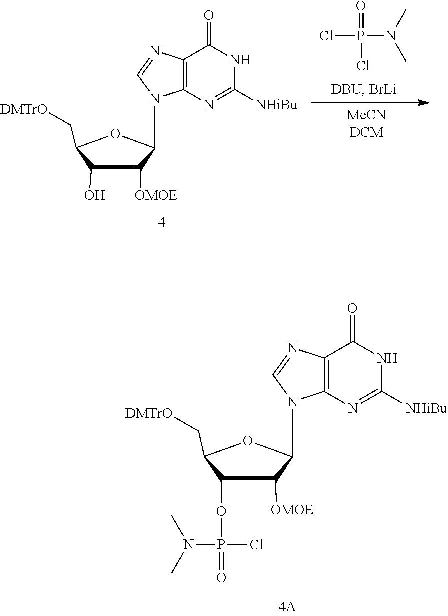

To a solution of compound 4 (10 g, 14.01 mmol, 1 eq.) in DCM (30 mL) and MeCN (90 mL) was added LiBr (3.89 g, 44.83 mmol, 1.13 mL, 3.2 eq.) and DBU (6.83 g, 44.83 mmol, 6.76 mL, 3.2 eq.), then was added N-dichlorophosphoryl-N-methyl-methanamine (3.40 g, 21.02 mmol, 1.5 eq.). The mixture was stirred at 0° C. for 2 hr. The reaction mixture was concentrated under reduced pressure at 0° C. to give a residue. The residue was purified by column chromatography (SiO₂, Petroleum ether/Ethyl acetate=1/0 to 0/1). Compound 4A (7 g, 8.34 mmol, 59.53% yield) was obtained as a white solid. LCMS: (M−H⁺): 837.3. TLC (Petroleum ether: Ethyl acetate=0:1), Rf=0.09.

Step 4H. Preparation of compound 8.

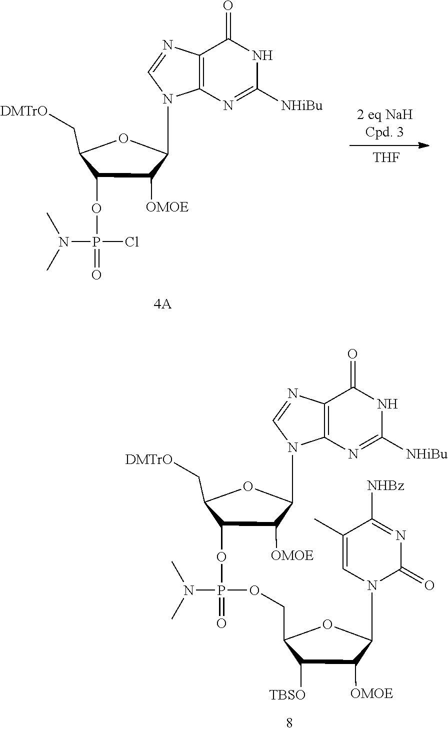

To a solution of compound 4A (4.45 g, 8.34 mmol, 1 eq.) in THF (50 mL) was added NaH (667.18 mg, 16.68 mmol, 60% purity, 2 eq.) at 0° C., then stirred 5min, compound 3 (7 g, 8.34 mmol, 28.20 uL, 1 eq.) in THF (50 mL) was added. The mixture was stirred at 0-15° C. for 2 hr. The reaction mixture was diluted with NH₄C1 20 mL and extracted with DCM 100 mL * 2, then dried over, filtered and concentrated under reduced pressure to give a residue. The crude product was purified by reversed-phase HPLC (column: Agela DuraShell C18 250*70 mm*10 um; mobile phase: [water (10 mM NH₄HCO3)-ACN]; B%: 75%-95%,@100 mL/min). Compound 8 (2.09 g, 1.56 mmol, 18.75% yield) was obtained as a white solid. LCMS: (M−H⁺):1334.1.

Step 5H. Preparation of compound WV-NU-186.

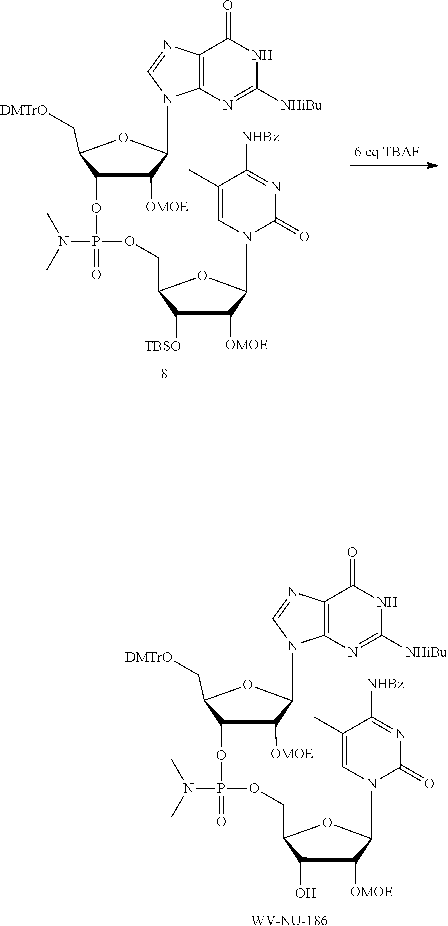

To a solution of compound 8 (10.00 g, 7.48 mmol, 1 eq.) in THF (20 mL) was added TBAF (1 M, 44.89 mL, 6 eq.). The mixture was stirred at 15° C. for 2 hr. The reaction mixture was concentrated under reduced pressure to give a residue. The residue was dissolved with Ethyl acetate 50 mL, then diluted with H₂O 50 mL*3. The combined organic layers were dried over filtered and concentrated under reduced pressure to give a residue. The crude product was purified by reversed-phase HPLC (column: Phenomenex Gemini C18 250*50 mm*10 um; mobile phase: [water (10 mM NH₄HCO₃)-ACN]; B%: 45%-75%,@100 mL/min). Compound WV-NU-186 (4.8 g, 3.93 mmol, 52.49% yield) was obtained as a white solid. LCMS: (M−H⁺): 1221.6.

Step 6H. Preparation of compounds WV-NU-186Sp and WV-NU-186Rp.

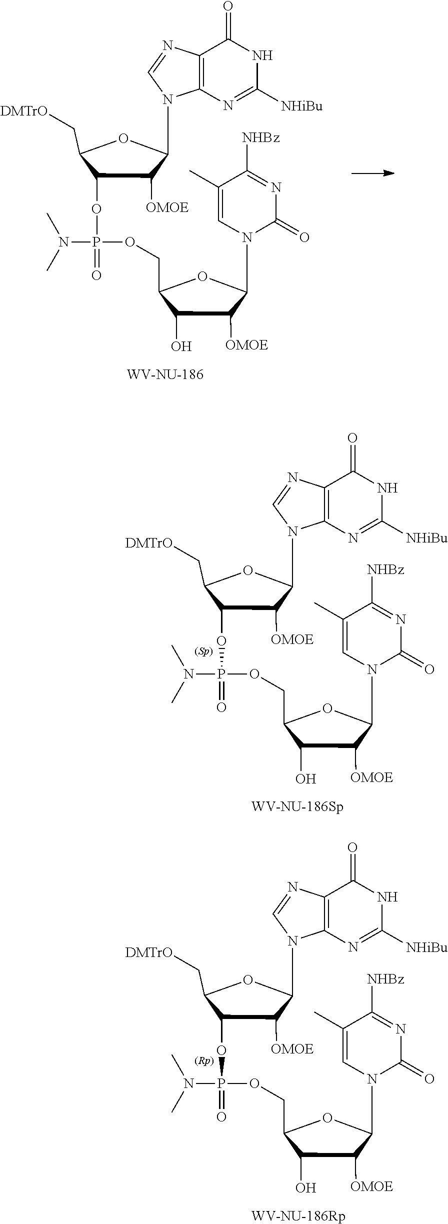

The crude product was purified by reversed-phase HPLC (column: Phenomenex Titank C18 Bulk 250*70 mm 10u; mobile phase: [water (10 mM NH₄HCO₃)-ACN]; B%: 60%-72%, 20min), after purification see. Compound WV-NU-186Rp (3.48 g, 2.85 mmol, 43.50% yield) and compound WV-NU-186Sp (3.06 g, 2.50 mmol, 38.25% yield) were obtained as white solids. WV-NU-186Rp: ⁱH NMR (400 MHz, DMSO-d₆) δ=12.98 (s, 1H), 12.06-11.97 (m, 1H), 11.57 (s, 1H), 8.19-8.11 (m, 3H), 7.75-7.66 (m, 1H), 7.63-7.56 (m, 1H), 7.53-7.45 (m, 2H), 7.37-7.31 (m, 2H), 7.30-7.16 (m, 8H), 6.89-6.79 (m, 5H), 5.93-5.89 (m, 1H), 5.88-5.84 (m, 1H), 5.34-5.29 (m, 1H), 4.98-4.90 (m, 1H), 4.85-4.80 (m, 1H), 4.27-4.20 (m, 3H), 4.09-4.01 (m, 3H), 3.88-3.80 (m, 1H), 3.73-3.69 (m, 8H), 3.45 (td, J=4.6, 17.4 Hz, 5H), 3.33 (s, 5H), 3.31-3.25 (m, 2H), 2.57 (s, 3H), 2.55 (s, 3H), 1.91-1.87 (m, 3H), 1.11 (dd, J=2.9, 6.8 Hz, 7H). LCMS: (M−H⁺): 1220.5. WV-NU-186Sp: ¹HNMR (400 MHz, DMSO-d₆) δ=12.95 (s, 1H), 12.11-12.05 (m, 1H), 11.65-11.55 (m, 1H), 8.23-8.09 (m, 3H), 7.79-7.72 (m, 1H), 7.63-7.45 (m, 3H), 7.39-7.34 (m, 2H), 7.31-7.20 (m, 7H), 6.85 (dd, J=3.8, 8.8 Hz, 4H), 6.00-5.85 (m, 2H), 5.36-5.28 (m, 1H), 4.93-4.78 (m, 2H), 4.41-4.35 (m, 1H), 4.15-3.98 (m, 5H), 3.78-3.66 (m, 9H), 3.65-3.59 (m, 1H), 3.52-3.45 (m, 2H), 3.12 (s, 3H), 2.77-2.70 (m, 1H), 2.66 (d, J=10.3 Hz, 6H), 2.09 (s, 6H), 2.02-1.95 (m, 3H), 1.12 (dd, J=2.9, 6.6 Hz, 6H). LCMS: (M−H⁺):1220.

Step 7H. Synthesis of WV-NU-186Rp-L-DPSE amidite.

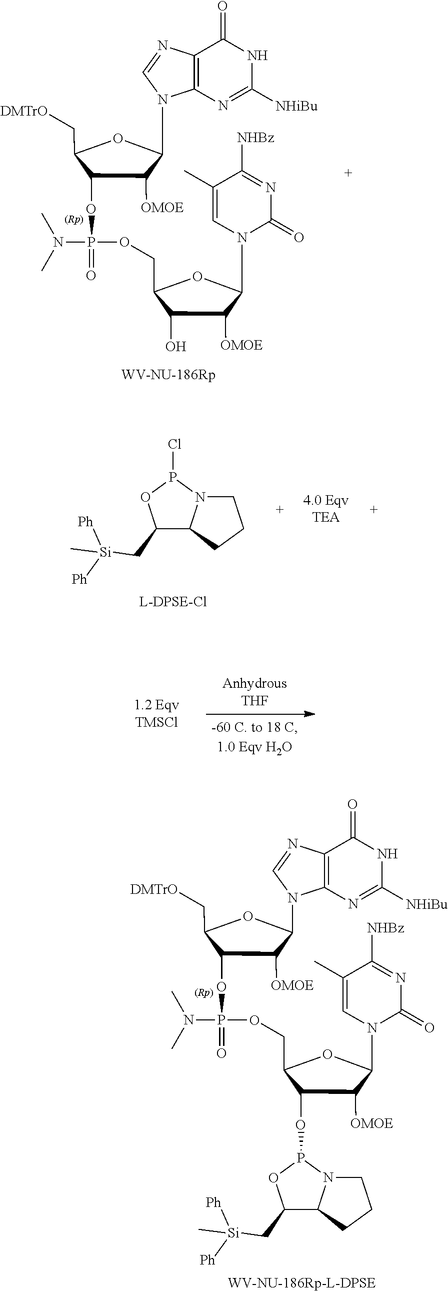

Compound WV-NU-186Rp (3.3 g, 2.7 mmol, 1.0 eq.) in a two necked flask (200 mL) was azeotroped three times with anhydrous toluene (30 mL) and was dried for 24 hrs on high vacuum. To the flask was added anhydrous THF (12 mL) under argon and solution was cooled to -60° C. to the reaction mixture was added triethyl amine (1.25 mL, 8.92 mmol, 4.0 eq.). To the reaction mixture was added TMSC1 (0.34 mL, 2.7 mmol, 1.0 eq.) followed by addition of DPSE-Cl (0.9 M) solution (5.4 mL, 6.0 mmol, 2.0 eq.) over the period of 5 min. The reaction mixture was warmed to room temperature and reaction progress was monitored by HPLC. After disappearance of starting material, reaction was quenched by addition of water and dried by addition of molecular sieve. The reaction mixture was filtered through fritted glass tube. Reaction flask and precipitate was washed with anhydrous THF (10 mL). Obtained filtrate was collected and solvent was removed under reduced pressure. The residue was purified by column chromatography (SiO₂, 50-100% Ethyl acetate (5% Et₃N) in Hexanes) to give WV-NU-186Rp-L-DPSE Amidite (3.3 g, 76% yield) as a white solid. Chemical Formula: C₇₉H₉₄N₁₀O₁₈P₂Si, Cacl. Mass (M−H⁺):1561.71. LCMS: (M−H⁺): 1560.34. ¹H NMR (600 MHz, CDCl₃) δ=8.90 (s, 1H), 8.17-8.12 (m, 2H), 7.61 (s, 1H), 7.39-7.30 (m, 7H), 7.28 (t, J=7.7 Hz, 2H), 7.22-7.12 (m, 8H), 7.09 (s, 3H), 7.11-6.99 (m, 6H), 6.62-6.55 (m, 4H), 5.73 (d, J=4.5 Hz, 1H), 5.65 (d, J=3.9 Hz, 1H), 5.50 (dt, J=9.8, 5.0 Hz, 1H), 4.85 (t, J=4.6 Hz, 1H), 4.73 (td, J=7.1, 5.4 Hz, 1H), 4.37 (dt, J=8.7, 5.4 Hz, 1H), 4.13 (dt, J=5.7, 3.2 Hz, 1H), 4.08 (ddd, J=11.6, 5.1, 2.6 Hz, 1H), 4.03-3.93 (m, 1H), 3.90 (dd, J=5.7, 2.8 Hz, 1H), 3.85 (dd, J=5.2, 3.9 Hz, 1H), 3.76 (ddd, J=11.6, 5.2, 3.0 Hz, 1H), 3.63-3.55 (m, 8H), 3.57-3.48 (m, 1H), 3.36-3.27 (m, 6H), 3.24-3.18 (m, 1H), 3.11 (s, 3H), 3.04 (s, 3H), 2.96 (dt, J=9.5, 3.1 Hz, 2H), 2.34 (d, J=10.3 Hz, 6H), 2.01-1.92 (m, 4H), 1.64 (dt, J=8.1, 4.0 Hz, 1H), 1.47 (td, J=13.7, 7.4 Hz, 2H), 1.29 (dd, J=14.6, 6.8 Hz, 1H), 1.22-1.17 (m, 1H), 1.12-1.02 (m, 1H), 0.90 (d, J=6.9 Hz, 3H), 0.83 (d, J=6.9 Hz, 3H), 0.47 (s, 3H). ¹³C NMR (151 MHz, CDCl₃) δ=178.73, 159.66, 158.77, 158.67, 155.54, 147.78, 147.73, 147.41, 144.39, 138.60, 137.65, 137.03, 136.36, 136.05, 135.59, 135.47, 134.47, 134.41, 134.37, 132.60, 130.11, 130.07, 130.00, 129.98, 129.94, 129.60, 129.54, 128.19, 128.14, 128.10, 128.07, 128.03, 127.99, 127.91, 127.11, 121.88, 113.36, 113.29, 113.17, 111.69, 90.02, 86.61, 86.43, 81.74, 81.69, 81.55, 81.53, 81.48, 81.44, 81.00, 80.99, 78.93, 78.87, 73.52, 73.49, 72.47, 72.36, 72.33, 72.17, 70.94, 70.59, 69.99, 69.90, 67.63, 67.61, 65.11, 65.07, 62.59, 60.40, 59.05, 59.01, 58.94, 58.92, 55.29, 55.28, 46.85, 46.61, 36.66, 36.56, 36.54, 36.27, 27.05, 25.93, 25.91, 21.07, 18.78, 18.71, 17.66, 17.63, 14.22, 13.47, −3.40. ³¹P NMR (243 MHz, CDCl₃) δ=155.61, 10.70.

Step 8H. Synthesis of WV-NU-186Sp-L-DPSE amidite.

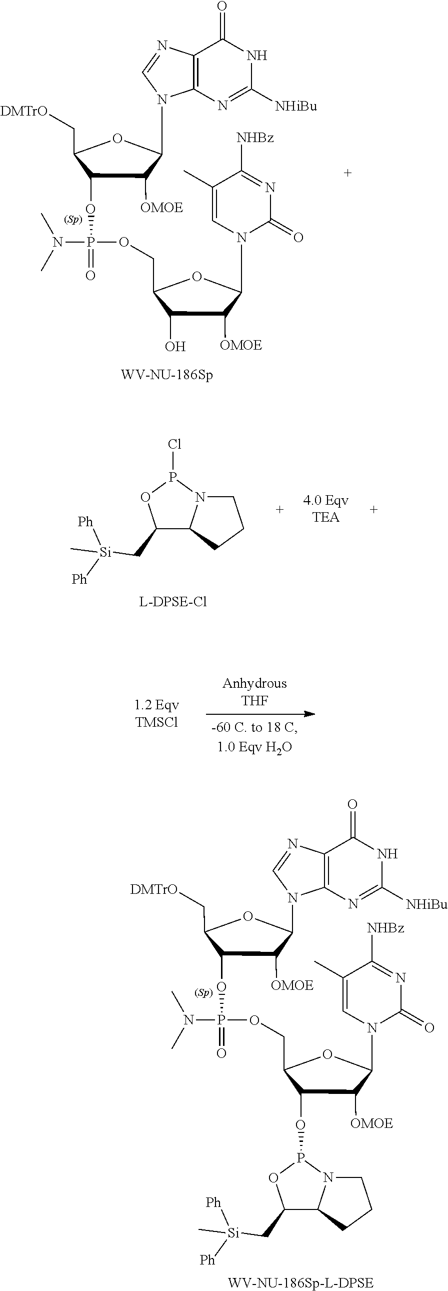

WV-NU-186Sp (3.0 g) of compound was converted into WV-NU-186Sp-L-DPSE amidite same as WV-NU-186Rp into WV-NU-186Rp-L-DPSE amidite (2.46 mg, 62% yield). Chemical Formula: C₇₉H₉₄N₁₀O₁₈P₂Si, Cacl. Mass (M−H⁺):1561.71. LCMS: (M−H⁺): 1560.34. ¹H NMR (600 MHz, CDCl₃) δ=8.12-8.08 (m, 1H), 7.56 (s, 1H), 7.36-7.29 (m, 4H), 7.24 (t, J=7.7 Hz, 2H), 7.18 (d, J=1.8 Hz, 1H), 7.18-7.11 (m, 4H), 7.11 (dd, J=4.1, 2.5 Hz, 2H), 7.05 (dd, J=6.7, 2.2 Hz, 2H), 7.02-6.96 (m, 2H), 6.99-6.93 (m, 1H), 6.56-6.49 (m, 3H), 5.66 (d, J=4.4 Hz, 1H), 5.62-5.58 (m, 1H), 5.36 (d, J=3.4 Hz, 1H), 4.78 (t, J=4.6 Hz, 1H), 4.71 (q, J=6.8 Hz, 1H), 4.29 (dt, J=9.2, 5.8 Hz, 1H), 4.15 (dt, J=5.3, 2.5 Hz, 1H), 3.92 (q, J=7.2 Hz, 1H), 3.83-3.70 (m, 3H), 3.64-3.52 (m, 1H), 3.53 (s, 4H), 3.51-3.40 (m, 1H), 3.37-3.29 (m, 1H), 3.31-3.20 (m, 4H), 3.10 (s, 2H), 3.04 (s, 2H), 3.08-2.97 (m, 1H), 2.94 (ddd, J=9.6, 4.1, 2.2 Hz, 1H), 2.52 (d, J=10.3 Hz, 4H), 1.84 (s, 1H), 1.64 (dt, J=8.2, 4.1 Hz, 1H), 1.51- 1.39 (m, 1H), 1.27 (dd, J=14.7, 6.7 Hz, 1H), 1.22-1.17 (m, 1H), 1.09-1.00 (m, 2H), 0.85 (d, J=6.9 Hz, 2H), 0.74 (d, J=6.9 Hz, 2H), 0.45 (s, 3H). ¹³C NMR (151 MHz, CDCl₃) δ −3.87, −3.39, 13.47, 14.22, 17.81, 17.84, 18.55, 18.79, 19.05, 21.07, 25.95, 25.97, 27.18, 36.20, 36.71, 36.73, 46.45, 46.69, 55.22, 55.25, 58.88, 58.97, 59.00, 59.03, 60.40, 61.98, 64.80, 64.84, 67.73, 69.60, 69.66, 70.45, 70.57, 70.65, 72.13, 72.16, 72.23, 72.32, 73.39, 73.42, 76.85, 76.88, 77.06, 77.09, 77.27, 77.30, 79.24, 79.30, 80.58, 81.20, 81.27, 81.32, 81.59, 81.63, 86.31, 86.59, 91.53, 111.59, 113.12, 113.18, 113.26, 122.04, 127.02, 127.86, 127.91, 127.97, 127.99, 128.12, 128.16, 128.18, 129.48, 129.53, 129.92, 129.97, 129.99, 130.03, 130.07, 130.11, 132.61, 134.37, 134.40, 134.42, 134.53, 135.61, 135.77, 136.01, 136.49, 137.03, 138.26, 138.98, 144.67, 147.37, 147.55, 147.78, 155.60, 158.59, 158.69, 159.68, 171.15, 178.75, 179.66, 180.34. ³¹P NMR (243 MHz, CDCl₃) δ=153.16, 11.22.

Certain useful compounds were prepared and described below:

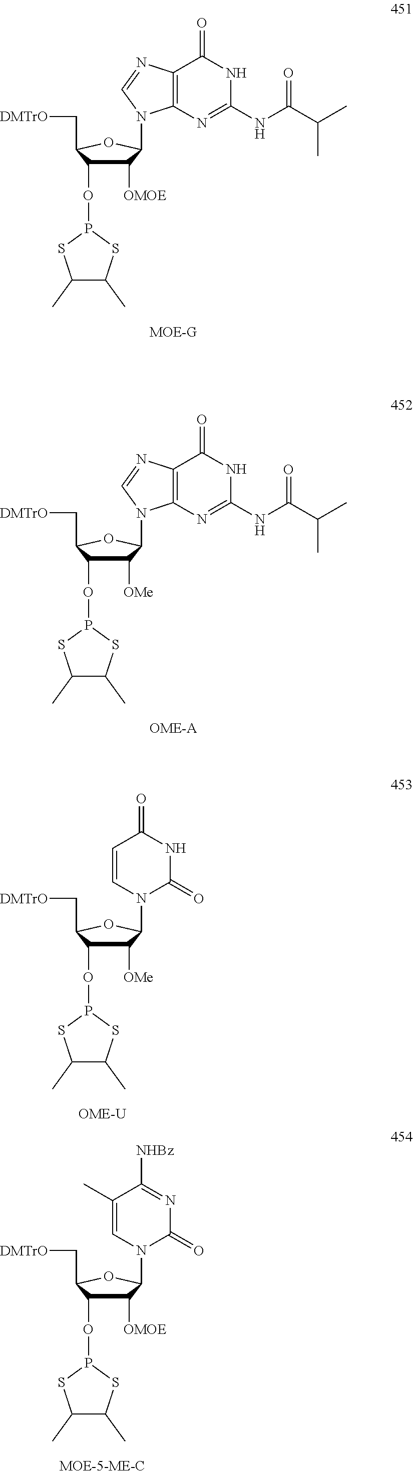

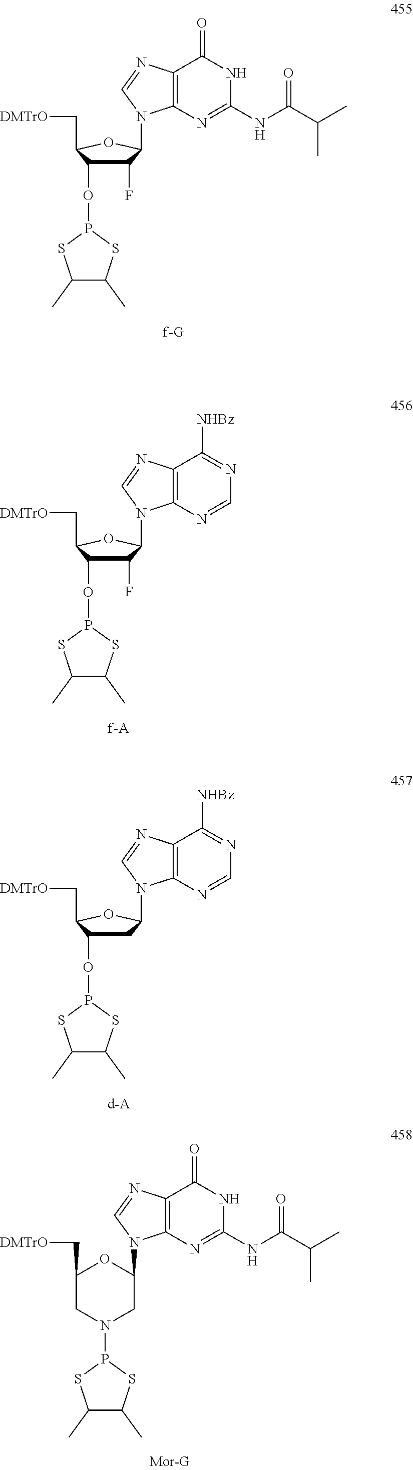

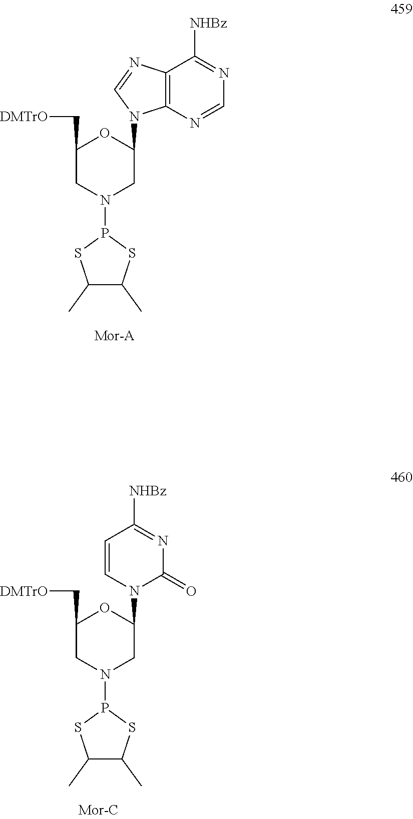

MOE-G monomer 451: Yield 81%. ³¹P NMR (243 MHz, CDCl₃) δ 175.14, 158.52, 150.30, 148.81; MS (ES) m/z calculated for C₄₂H50N5O₉PS₂ [M+H]⁺864.29, Observed: 864.56 [M+H]⁺.

OMe-A monomer 452: Yield 92%. ³¹P NMR (243 MHz, CDCl₃) δ 175.65, 159.27, 151.04, 150.10; MS (ES) m/z calculated for C₄₃H₄₄N₅O₇PS₂ [M+H]⁺838.25, Observed: 838.05 [M+H]⁺.

OMe-U monomer 453: Yield 94%. ³¹P NMR (243 MHz, CDCl₃) δ 175.09, 162.04, 154.12, 153.58; MS (ES) m/z calculated for C₃₅H₃₉N₂O₈PS₂ [M+K]⁺749.15, Observed: 749.06 [M+K]⁺.

MOE—S-Me-C monomer 454: Yield 91%. ³¹P NMR (243 MHz, CDCl₃) δ 175.53, 162.04, 153.78, 153.61; MS (ES) m/z calculated for C₄₅H50N₃O₉PS₂ [M+H]⁺872.28, Observed: 872.16 [M+K]⁺.

f-G monomer 455: Yield 97%. ³¹P NMR (243 MHz, CDCl₃) δ 176.88 (d), 161.94 (d), 154.16 (d), 152.48 (d); MS (ES) m/z calculated for C₃₉H₄₃FN₅O₇PS₂ [M+H]⁺808.24, Observed: 808.65 [M+H]⁺.

f-A monomer 456: Yield 99%. ³¹P NMR (243 MHz, CDCl₃) δ 177.43 (d), 159.63 (d), 149.76 (d), 149.55 (d); MS (ES) m/z calculated for C₄₂H₄₁FN₅O₆PS₂ [M+H]⁺826.23, Observed: 826.56 [M+H]⁺.

dA monomer 457: Yield 98%. ³¹P NMR (243 MHz, CDCl₃) δ 171.85, 154.47, 146.19, 144.48; MS (ES) m/z calculated for C₄₂H₄₂N₅O₆PS₂ [M+K]⁺846.20, Observed: 846.56 [M+K]⁺.

Mor-G monomer 458: Yield 72%. ³¹P NMR (243 MHz, CDCl₃) δ 121.26, 105.98, 93.48, 93.24; MS (ES) m/z calculated for C₃₉H₄₅N₆O₆PS₂[M+K]⁺827.22, Observed: 827.60 [M+K]⁺.

Mor-A monomer 459: Yield 37%. ³¹P NMR (243 MHz, CDCl₃) δ 121.87, 106.17, 93.23, 93.05; MS (ES) m/z calculated for C₄₂H₄₃N₆O₅PS₂ [M+K]⁺845.21, Observed: 845.32 [M+K]⁺.

Mor-C monomer 460: Yield 68%. ³¹P NMR (243 MHz, CDCl₃) δ 122.34, 106.05, 93.33, 92.6116; MS (ES) m/z calculated for C₄₁H₄₃N₄O₆PS₂ [M+K]⁺821.20, Observed: 821.54 [M+K]⁺.

In some embodiments, various stereopure morpholine monomers were prepared as described below:

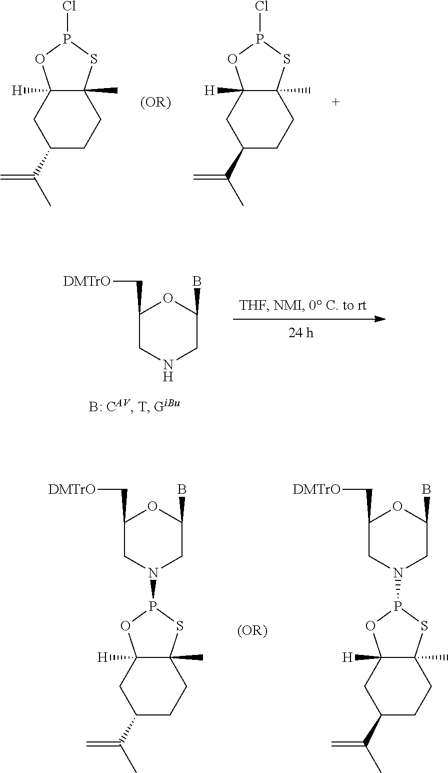

The 5′-ODMTr protected morpholine nucleoside (11.1 mmol) was dried in a three neck 250 mL round bottom flask by co-evaporating with anhydrous toluene (100 mL) followed by under high vacuum for 18 h. The dried nucleoside was dissolved in dry THF (55 mL) under argon atmosphere. Then, 1-methylimidazole (44.2 mmol, 4 equiv.) was added into the reaction mixture, then cooled to ˜−10° C. [at this stage, if B: G^{B}″ chrorotrimethylsilane (0.9 equiv.) was added]. A THF solution of the crude chloro reagent (1 M solution, 1.8 equiv., 19.9 mmol) was added to the above mixture through cannula over ˜3 min, then, gradually warmed to room temperature over about 1 h. LCMS showed that the starting material was consumed. Resulting reaction mixture was stirred for additional 24 h at rt. Then filtered carefully under vacuum/argon and the resulting filtrate was concentrated under reduced pressure to give a yellow foam which was further dried under high vacuum overnight. Crude mixture was purified by silica gel column [Column was pre-deactivated using acetonitrile then ethyl acetate (5% TEA) and then equilibrated using ethyl acetate-hexanes] chromatography using ethyl acetate and hexane as eluents.

Structure of certain stereopure morpholine monomers are described below:

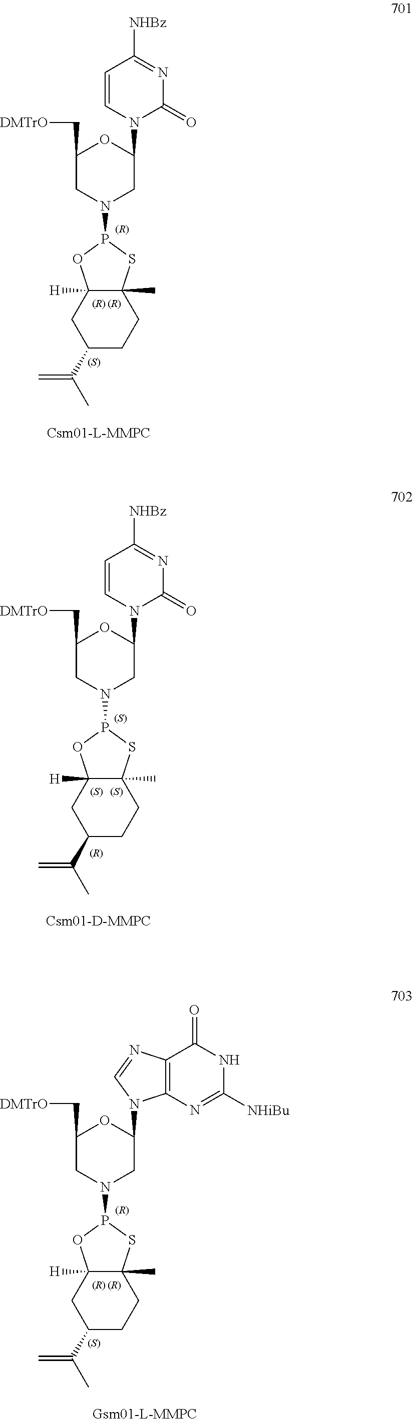

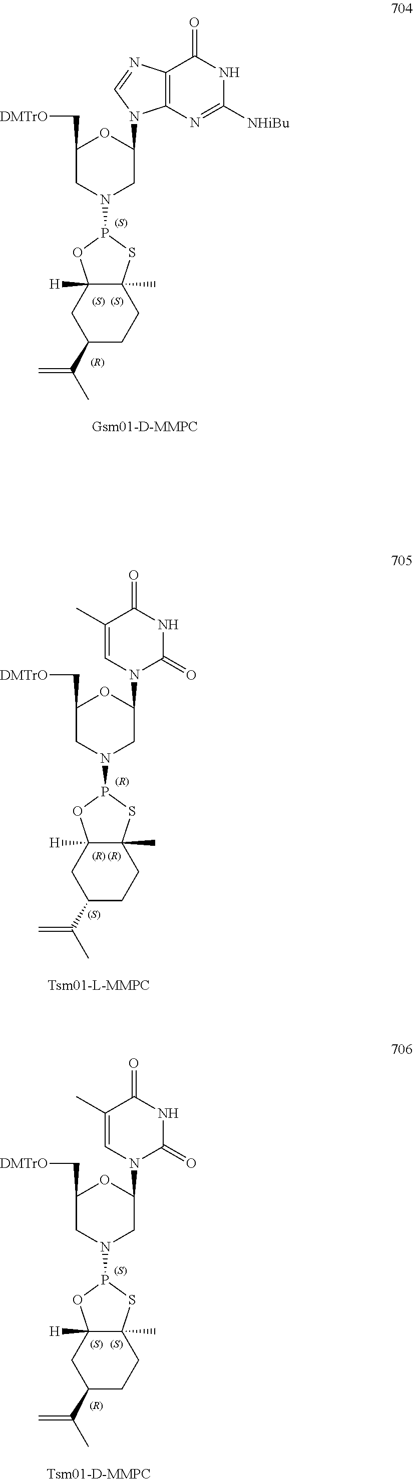

Stereopure (Rp) Csm01-L-MMPC monomer 701: Yield 39%. ³¹P NMR (243 MHz, CDCl₃) δ 137.80; MS (ES) m/z calculated for C₄₇H_{5,}N4O7PS [M+K]⁺885.29, Observed: 885.51 [M+K]⁺.

Stereopure (Sp) Csm01-D-MMPC monomer 702: Yield 28%. ³¹P NMR (243 MHz, CDCl₃) δ 137.42; MS (ES) m/z calculated for C₄₇H₅IN407PS [M+K]⁺885.29, Observed: 885.70 [M+K]⁺.

Stereopure (Rp) Gsm01-L-MMPC monomer 703: Yield 37%. ³¹P NMR (243 MHz, CDCl₃) δ 136.58; MS (ES) m/z calculated for C₄₅H₅₅N₆O₆PS [M+K]⁺891.31, Observed: 891.48 [M+K]⁺.

Stereopure (Sp) Gsm01-D-MMPC monomer 704: Yield 38%. ³¹1³NMR (243 MHz, CDCl₃) δ 136.56; MS (ES) m/z calculated for C₄₅H₅₅N₆O₆PS [M+K]⁺891.31, Observed: 891.67 [M+K]⁺.

Stereopure (Rp) Tsm01-L-MMPC monomer 705: Yield 30%. ³¹P NMR (243 MHz, CDCl₃) δ 138.52; MS (ES) m/z calculated for C₄it148N₂O₇PS [M+Na]⁺780.28, Observed: 780.52 [M+Na]⁺.

Stereopure (Sp) Tsm01-D-MMPC monomer 706: Yield 25%. ³¹P NMR (243 MHz, CDCl₃) δ 137.62; MS (ES) m/z calculated for C₄H₄₈N₂O₇PS [M+Na]⁺780.28, Observed: 780.81 [M+Na]⁺.

In some embodiments, the following abbreviations are used:

### ABBREVIATION

1X reagent: TEA-3HF : TEA : H₂O : DMSO=5.0 : 1.8 : 15.5 : 77.7 (v/v/v/v)
ADIH: 2-azido-1,3-dimethylimidazolium hexafluorophosphate
CMIMT: N-cyanomethylimidazolium triflate
CPG: controlled pore glass
DBU: 1,8-diazabicyclo [5.4.0]undec-7-ene
DCM: dichloromethane, CH₂Cl₂
DIPEA: diisopropylethylamine
DMSO: dimethylsulfoxide
DMTr: 4,4′-dimethoxytrityl
GalNAc: N-acetylgalactosamine
HF: hydrogen fluoride
HATU: 1-[bis(dimethylamino)methylene]-1H-1,2,3-triazolo[4,5-b]pyridinium 3-oxide
hexafluorophosphate
IBN: isobutyronitrile
MeCN: acetonitrile
Melm: N-methylimidazole
TCA: trichloroacetic acid
TEA: triethylamine
XH: xanthane hydride

### Example 25. Certain Additional Chemical Moieties

As described herein, an oligonucleotide of the present disclosure may comprise one or more additional chemical moiety. In some embodiments, an additional chemical moiety is or comprises an ASGPR ligand, e.g.,

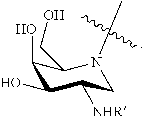

In some embodiments, a ligand is or comprises

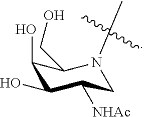

Various technologies are available for preparing and conjugating additional chemical moieties in accordance with the present disclosure. Certain technologies are described below as examples.

### Synthesis of 5-((2R,3S,4R,5S)-5-acetamido-3,4-bis(benzoyloxy)-2-((benzoyloxy)methyl)piperidin-1-yl)-5-oxopentanoic acid (e.g., for Mod152)

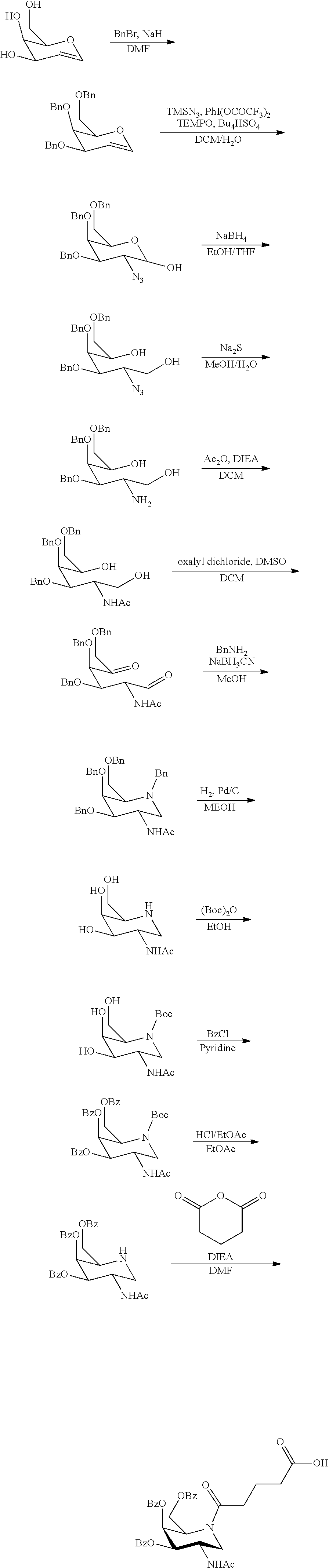

Step 1: Two batches in parallel: To a solution of (2R,3R,4R)-2-(hydroxymethyl)-3,4-dihydro-2H-pyran-3,4-diol (75 g, 513.20 mmol, 1 eq.) in DMF (2250 mL) was added NaH (92.37 g, 2.31 mol, 60% purity, 4.5 eq.) at 0° C., then added BnBr (307.21 g, 1.80 mol, 213.34 mL, 3.5 eq.). The mixture was stirred at 0-20° C. for 0.5 hr. TLC (Petroleum ether : Ethyl acetate=10:1, R_{f}=0.40) indicated starting material was consumed and two new spots formed. The reaction mixture was quenched by sat. NH₄Cl (1500 mL) at 0° C., extracted with MTBE (1500 mL×3), dried over Na₂SO₄, filtered and concentrated under reduced pressure to give a residue. The residue was purified by column chromatography (SiO₂, Petroleum ether/Ethyl acetate=1/0 to 0:1) to get 318 g (2R,3R,4R)-3,4-bis(benzyloxy)-2-((benzyloxy)methyl)-3,4-dihydro-2H-pyran as a yellow solid. MS: 439.1 (M=Na)⁺; TLC (Petroleum ether : Ethyl acetate=10:1) R_{f}=0.40.

Step 2: Fifteen batches in parallel: To a mixture of (2R,3R,4R)-3,4-bis(benzyloxy)-2-((benzyloxy)methyl)-3,4-dihydro-2H-pyran (30 g, 72.03 mmol, 1 eq.) and TMSN₃ (24.89 g, 216.08 mmol, 28.42 mL, 3 eq.) in DCM (1800 mL) was added PIFA (68.83 g, 144.05 mmol, 90% purity, 2 eq.), TEMPO (2.27 g, 14.41 mmol, 0.2 eq.), Bu4NHSO₄ (4.89 g, 14.41 mmol, 0.2 eq.) and H₂O (64.90 g, 3.60 mol, 64.90 mL, 50 eq.) sequentially without any intervening time at 0-5° C. The mixture was stirred at 0-5° C. for 40 mins. TLC (Petroleum ether/Ethyl acetate=3:1, R_{f}=0.35) showed that the starting material was consumed completely. The mixture was quenched by saturated aq. NaHCO3 (1500 mL) and the aqueous phase was extracted with dichloromethane (500 mL×3). The organic phase was washed by H₂O (1000 mL×3) and saturated aq. NaCl (1000 mL×3), dried over Na₂SO₄. The fifteen batches were concentrated under reduced pressure to remove the solvent. The crude product was purified by MPLC (SiO₂, Ethyl acetate/Petroleum ether=0% to 20%) to obtain (2R,3R,4R,5R,6R)-3-azido-4,5-bis(benzyloxy)-6-((benzyloxy)methyl)tetrahydro-2H-pyran-2-ol (280 g, crude) as yellow oil. LCMS: M+Na⁺=498.1, purity: 63.34%; TLC (Petroleum ether/Ethyl acetate=3:1) R_{f}=0.35.

Step 3: Two batches in parallel: To a solution of (2R,3R,4R,5R,6R)-3-azido-4,5-bis(benzyloxy)-6-((benzyloxy)methyl)tetrahydro-2H-pyran-2-ol (140 g, 294.41 mmol, 1 eq.) in EtOH (2000 mL) was added NaBH₄ (16.64 g, 439.86 mmol, 1.49 eq.) at 0-5° C. and the mixture was stirred at 20-25° C. for 1 hr. TLC (Petroleum ether/Ethyl acetate=2:1, R_{f}=0.45) and LCMS showed that the starting material was consumed completely. The mixture was quenched by aq. NH₄Cl (1500 mL) and concentrated under reduced pressure to remove the most solvent, then extracted with ethyl acetate (500 mL×3). The two batches were combined and the organic phase was dried over anhydrous Na₂SO₄ and concentrated under reduced pressure to remove the solvent. The crude product was purified by MPLC (SiO₂, Ethyl acetate/Petroleum ether=20% to 50%) to obtain 2-azido-3,4,6-tris(benzyloxy)hexane-1,5-diol (219 g, crude) as white solid. LCMS: M+Na⁺=500.1; TLC (Petroleum ether/Ethyl acetate=2:1) R_{f}=0.45.

Step 4. Three batches in parallel: To a solution of 2-azido-3,4,6-tris(benzyloxy)hexane-1,5-diol (96 g, 201.03 mmol, 1 eq.) in MeOH (2000 mL) and H₂O (400 mL) was added Na₂S·9H20 (241.41 g, 1.01 mol, 168.82 mL, 5 eq.) and stirred at 70° C. for 12 hrs. TLC (Petroleum ether/Ethyl acetate=2:1, R_{f}=0) showed that the starting material was consumed completely. The mixture was filtered and concentrated under reduced pressure to remove the solvent. The crude product was used for the next step without any purification. 2-amino-3,4,6-tris(benzyloxy)hexane-1,5-diol (272.32 g, crude) was obtained as yellow solid.

Step 5. Three batches in parallel: To a solution of 2-amino-3,4,6-tris(benzyloxy)hexane-1,5-diol (90 g, 199.31 mmol, 1 eq.) in DCM (1000 mL) was added DIEA (51.52 g, 398.62 mmol, 69.43 mL, 2 eq.) at 0-5° C., followed by Ac₂O (26.45 g, 259.11 mmol, 24.27 mL, 1.3 eq.). The mixture was stirred at 5-10° C. for 3 hrs. LCMS showed that the starting material was consumed completely. The mixture was filtered and combined, then concentrated under reduced pressure to remove the solvent. TLC (Petroleum ether/Ethyl acetate=0:1, R_{f}=0.35) showed the desired product. The crude product was purified by MPLC (SiO₂, Ethyl acetate/Petroleum ether=0% to 50%) to obtain N-(3,4,6-tris(benzyloxy)-1,5-dihydroxyhexan-2-yl)acetamide (176 g, 356.57 mmol, 59.63% yield) as white solid. ¹FINMR (400 MHz, CHLOROFORM-d) δ=7.42-7.28 (m, 15H), 6.16 (br d, J=8.7 Hz, 1H), 4.75 (d, J=11.0 Hz, 1H), 4.66-4.43 (m, 5H), 4.43-4.36 (m, 1H), 4.08-4.00 (m, 1H), 3.89 (dd, J=1.6, 7.9 Hz, 1H), 3.75-3.65 (m, 2H), 3.63-3.48 (m, 3H), 2.50 (d, J=8.7 Hz, 1H), 2.41 (dd, J=5.1, 6.8 Hz, 1H), 1.95 (s, 3H); LCMS: M +H⁺=494.1.

Step 6: Three batches in parallel: To a solution of oxalyl dichloride (67.12 g, 528.78 mmol, 46.29 mL, 4.5 eq.) in DCM (450 mL) was added DMSO (55.08 g, 705.04 mmol, 55.08 mL, 6 eq.) in DCM (150 mL) dropwised at −78-68° C. over 15 mins, and the mixture was stirred for 0.5 hr. N-(3,4,6-tris(benzyloxy)-1,5-dihydroxyhexan-2-yl)acetamide (58 g, 117.51 mmol, 1 eq.) in DCM (300 mL) was added to the above mixture dropwised and stirred at −78-68° C. for 0.5 hr. The mixture was quenched by TEA (166.47 g, 1.65 mol, 228.98 mL, 14 eq.) at −78-68° C. and the mixture was stirred for 0.5 hr, then warmed to 5-10° C. (room temperature). LCMS showed that the starting material was consumed completely. The mixture was washed by H₂O (500 mL) and aq. NaCl (500 mL×2). The organic phase was dried over anhydrous Na₂SO₄ and concentrated under redeced pressure to remove the part solvent. The crude product was used respectively for the next step without any purification. N-(3,4,6-tris(benzyloxy)-1,5-dioxohexan-2-yl)acetamide (172.58 g, crude) was obtained as yellow liquid (in DCM). LCMS: M+H⁺=490.1, purity: 34.07%.

Step 7: Three batches in parallel: To a solution of N-(3,4,6-tris(benzyloxy)-1,5-dioxohexan-2-yl)acetamide (57.53 g, 117.51 mmol, 1 eq.) in DCM (900 mL) was added phenylmethanamine (13.85 g, 129.27 mmol, 14.09 mL, 1.1 eq.) in MeOH (900 mL), followed by NaBH₃CN (14.77 g, 235.03 mmol, 2 eq.) at 5-10° C. The mixture was stirred at 5-10° C. for 12 hrs. LCMS showed that the starting material was consumed completely. The mixture was filtered and concentrated under reduced pressure to remove the solvent. The residue was combined. TLC (Petroleum ether/Ethyl acetate=1:1, R_{f}=0.35) showed that the desired product was formed. The product was purified by MPLC (SiO₂, Ethyl acetate/Petroleum ether=30% to 45%) to give N-((3S,4R,5S,6R)-1-benzyl-4,5-bis(benzyloxy)-6-((benzyloxy)methyl)piperidin-3-yl)acetamide (46 g, 75.33 mmol, 21.37% yield, 92.477% purity) as white solid. ¹H NMR (400 MHz, METHANOL-d₄) δ=7.40-7.17 (m, 20H), 4.78-4.42 (m, 5H), 4.34-4.25 (m, 1H), 4.06 (br s, 1H), 3.95-3.87 (m, 1H), 3.82-3.64 (m, 3H), 3.49 (br d, J=6.8 Hz, 1H), 3.12-2.92 (m, 1H), 2.84 (dd, J=3.7, 12.3 Hz, 1H), 2.09 (br dd, J=7.5 , 12.1 Hz, 1H), 1.90-1.84 (m, 3H); LCMS: M +H⁺=565.1, purity: 92.47%.

Step 8: A mixture of N-((3S,4R,5S,6R)-1-benzyl-4,5-bis(benzyloxy)-6-((benzyloxy)methyl)piperidin-3-yl)acetamide (20 g, 35.42 mmol, 1 eq.) and Pd/C (80 g, 10% purity) in MeOH (500 mL) was evacuated in vacuo and backfilled with H₂ (50 Psi) three times, then stirred at 40-45° C. for 24 hrs. LCMS showed that the starting material was consumed completely. The mixture was filtered and concentrated under reduced pressure to remove the solvent. The crude product was used for the next step without any purification. N-((3S,4R,5S,6R)-4,5-dihydroxy-6-(hydroxymethyl)piperidin-3-yl)acetamide (8.02 g, crude) was obtained as gray solid.

Step 9: To a solution of N-((3S,4R,5S,6R)-4,5-dihydroxy-6-(hydroxymethyl)piperidin-3-yl)acetamide (8.02 g, 35.40 mmol, 1 eq.) in EtOH (120 mL) was added Boc2O (8.50 g, 38.94 mmol, 8.95 mL, 1.1 eq.) and stirred at 50° C. for 12 hours. LCMS showed that the starting material was consumed completely. The mixture was concentrated under reduced pressure to remove the solvent. TLC (Methanol/Dichloromethane=10:1, R_{f}=0.30) showed that the desired product was formed. The crude product was purified by MPLC (SiO₂, Methanol/Dichloromethane=0% to 6%) to obtain tert-butyl (2R,3S,4R,5S)-5-acetamido-3,4-dihydroxy-2-(hydroxymethyl)piperidine-1-carboxylate (9.27 g, 30.46 mmol, 86.04% yield) as white solid. LCMS: M+Na⁺=327.1, purity: 92.22%.

Step 10: To a solution of tert-butyl (2R,3S,4R,5S)-5-acetamido-3,4-dihydroxy-2-(hydroxymethyl)piperidine-1-carboxylate (10 g, 32.86 mmol, 1 eq.) in PYRIDINE (100 mL) was added BzCl (15.24 g, 108.43 mmol, 12.60 mL, 3.3 eq.) at 0-5° C. and stirred at 10-15° C. for 1 hr. LCMS showed that the starting material was consumed completely and desired product was detected. The mixture was diluted with ethyl acetate (500 mL) and washed by aq. HCl (1 M, 500 mL×3), saturated aq. NaHCO₃ (500 mL×3) and saturated aq. NaCl (500 mL×3). The organic phase was dried over anhydrous Na₂SO₄ and concentrated under reduced pressure to remove the solvent. TLC (Petroleum ether/Ethyl acetate=1:2, R_{f}=0.35) showed that the desired product was formed. The crude product was purified by MPLC (SiO₂, Ethyl acetate/Petroleum ether=0% to 30%) to obtain (2R,3S,4R,5S)-5-acetamido-2-((benzoyloxy)methyl)-1-(tert-butoxycarbonyl)piperidine-3,4-diyldibenzoate (19.21 g, 31.15 mmol, 94.81% yield) as white solid. LCMS: M−100+H⁺=517.0.

Step 11: To a solution of (2R,3S,4R,5S)-5-acetamido-2-((benzoyloxy)methyl)-1-(tert-butoxycarbonyl)piperidine-3,4-diyl dibenzoate (19.2 g, 31.14 mmol, 1 eq.) in EtOAc (200 mL) was added HCl/EtOAc (4 M, 200 mL, 25.69 eq.) at 0-5° C. and stirred at 5-10° C. for 12 hrs. LCMS showed that the starting material was consumed completely. The mixture was concentrated under reduced pressure to remove the solvent. The crude product was used for the next step without any purification. (2R,3S,4R,55)-5-acetamido-2-((benzoyloxy)methyl)-1-(tert-butoxycarbonyl)piperidine-3,4-diyl dibenzoate (16.34 g, 28.94 mmol, 92.94% yield, 97.937% purity, HCl) was obtained as white solid. ¹HNMR (400 MHz, METHANOL-d₄) δ=8.11 (br d, J=7.3 Hz, 2H), 7.96 (br d, J=7.5 Hz, 2H), 7.80 (br d, J=7.5 Hz, 2H), 7.65-7.49 (m, 3H), 7.43 (br t, J=7.5 Hz, 2H), 7.32 (q, J=7.3 Hz, 4H), 6.31 (br s, 1H), 5.68-5.55 (m, 1H), 5.00-4.88 (m, 1H), 4.78-4.64 (m, 2H), 4.52 (br s, 1H), 3.77 (br dd, J=4.5, 12.5 Hz, 1H), 3.52 (br t, J=12.5 Hz, 1H), 1.91 (s, 3H); LCMS: M +H⁺=517.0, purity: 97.93%.

Step 12: To a mixture of (2R,3S,4R,5S)-5-acetamido-2-((benzoyloxy)methyl)-1-(tert-butoxycarbonyl)piperidine-3,4-diyl dibenzoate (8 g, 14.47 mmol, 1 eq., HCl) and tetrahydropyran-2,6-dione (4.13 g, 36.17 mmol, 2.5 eq.) in DMF (70 mL) was added DIEA (9.35 g, 72.33 mmol, 12.60 mL, 5 eq.) at 5-10° C. The mixture was stirred at 85° C. for 12 hrs. LCMS showed that the starting material was consumed mostly. The mixture was concentrated under reduced pressure to remove the solvent. The crude product was detected by HPLC. The crude product was purified by prep-HPLC (HCl, MeCN/H₂O) to obtain 5-((2R,3 S,4R,5 S)-5-acetamido-3,4-bis (benzoyloxy)-2-((benzoyloxy)methyl)piperidin-1-yl)-5-oxopentanoic acid (5.31 g, 8.41 mmol, 58.13% yield, 99.878% purity) as yellow solid. ¹H NMR (400 MHz, DMSO-d₆) δ=12.05 (br s, 1H), 8.57 (br d, J=7.7 Hz, 1H), 8.08 (br d, J=7.1 Hz, 2H), 7.94-7.80 (m, 4H), 7.76-7.69 (m, 1H), 7.67-7.55 (m, 4H), 7.47 (br d, J=7.3 Hz, 4H), 5.84-5.65 (m, 1H), 5.56-5.22 (m, 2H), 4.99 (br t, J=10.1 Hz, 1H), 4.60 (br d, J=8.4 Hz, 1H), 4.41 (br d, J=14.6 Hz, 1H), 4.29 (br s, 1H), 4.00-3.74 (m, 2H), 2.42-2.31 (m, 1H), 2.24 (br d, J=5.3 Hz, 2H), 1.92 (s, 3H), 1.71 (br d, J=6.4 Hz, 2H); ¹³C NMR (101 MHz, DMSO-d₆) δ=174.77, 172.47, 170.07, 166.04, 165.28, 164.96, 134.36, 134.24, 133.76, 129.65, 129.42, 129.60 (br dd, J=20.9, 45.8 Hz, 1C), 129.02, 70.30, 67.58, 60.59, 49.08, 47.87, 41.40, 33.32, 32.46, 22.92, 20.53; LCMS: M +H⁺=631.3, purity: 99.87%.

### Synthesis of 5-((2R,3 S,4R,5 S)-5-acetamido-3,4-bis (benzoyloxy)-2-((benzoyloxy)methyl)piperidin-1-yl)pentanoic acid (e.g., for Mod154)

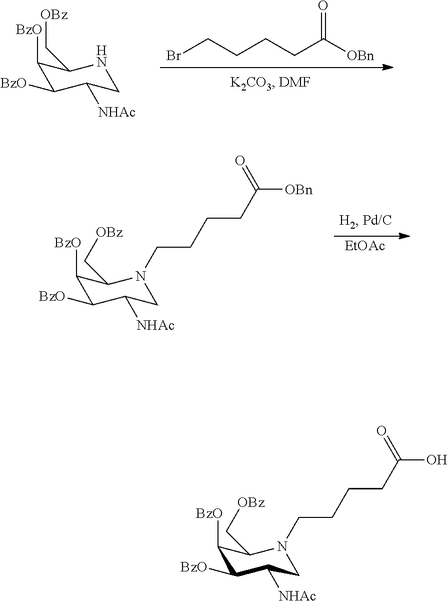

Step 1: To a mixture of (2R,3S,4R,5S)-5-acetamido-2-((benzoyloxy)methyl)piperidine-3,4-diyl dibenzoate (6 g, 10.85 mmol, 1 eq., HCl) and 5-bromopentanoic acid--benzyl 5-bromopentanoate (11.78 g, 32.55 mmol, 3 eq.) in DMF (60 mL) was added KI (360.22 mg, 2.17 mmol, 0.2 eq.) and DIEA (7.01 g, 54.25 mmol, 9.45 mL, 5 eq.) at 5-10° C. The mixture was stirred at 100° C. for 24 hrs. LCMS showed that the starting material was consumed mostly and desired product was detected. The mixture was concentrated under reduced pressure to remove the solvent. The crude product was detected by HPLC and purified by prep-HPLC (HCl, MeCN/H20) to obtain (2R,3S,4R,5S)-5-acetamido-2-((benzoyloxy)methyl)-1-(5-(benzyloxy)-5-oxopentyl)piperidine-3,4-diyl dibenzoate (7.5 g, 9.83 mmol, 90.62% yield, 92.655% purity) as yellow solid. MS: 707.1 (M+H)⁺.

Step 2: A mixture of (2R,3S,4R,5S)-5-acetamido-2-((benzoyloxy)methyl)-1-(5-(benzyloxy)-5-oxopentyl)piperidine-3,4-diyl dibenzoate (7.8 g, 11.04 mmol, 1 eq.) and Pd/C (8 g, 11.04 mmol, 10% purity, 1.00 eq.) in EtOAc (80 mL) was evacuated in vacuo and backfilled with H₂ (15 Psi) three times, then stirred at 10-15° C. for 6 hrs. LCMS showed that the starting material was consumed completely. The mixture was filtered and the filtrate was concentrated under reduced pressure to remove the solvent. The crude product was purified by prep-HPLC (column: Phenomenex luna C18 250*50 mm*10 um; mobile phase: [water (0.05%HCl)-ACN]; B%: 35%-55%, 20min) to obtain 5-((2R,3S,4R,5S)-5-acetamido-3,4-bis(benzoyloxy)-2-((benzoyloxy)methyl)piperidin-1-yl)pentanoic acid (2.83 g, 4.59 mmol, 41.58% yield) as white solid. ¹H NMR (400 MHz, METHANOL-d₄) δ=8.10-8.04 (m, 2H), 7.95-7.90 (m, 2H), 7.82-7.77 (m, 2H), 7.64-7.50 (m, 3H), 7.48-7.42 (m, 2H), 7.40-7.30 (m, 4H), 6.29-6.17 (m, 1H), 5.50-5.38 (m, 1H), 4.86-4.79 (m, 2H), 4.67-4.54 (m, 1H), 4.22-4.04 (m, 1H), 3.75-3.61 (m, 1H), 3.43-3.34 (m, 1H), 3.28-3.11 (m, 2H), 2.43-2.35 (m, 2H), 1.93-1.79 (m, 5H), 1.75-1.62 (m, 2H); ¹³C NMR (101 MHz, METHANOL-d₄) δ=175.50, 172.28, 165.74, 165.61, 165.47, 133.61, 133.28, 129.77, 129.39, 129.22, 128.96, 128.78, 128.65, 128.35, 128.19, 128.16, 68.65, 60.99, 60.42, 53.18, 52.53, 44.62, 32.78, 21.79, 21.22; LCMS: M+H⁺=617.3, purity: 98.62%.

### Synthesis of 1-((2R,3 S,4R,5 S)-5-acetamido-3,4-bis (benzoyloxy)-2-((benzoyloxy)methyl)piperidin-1-yl)-16,16-bis((3-((3-(5-((2R,3S,4R,5S)-5-acetamido-3,4-bis(benzoyloxy)-2-((benzoyloxy)methyl)piperidin-1-yl)pentanamido)propyl)amino)-3-oxopropoxy)methyl)-5,11,18-trioxo-14-oxa-6,10,17-triazanonacosan-29-oic acid (e.g., for Mod 155)

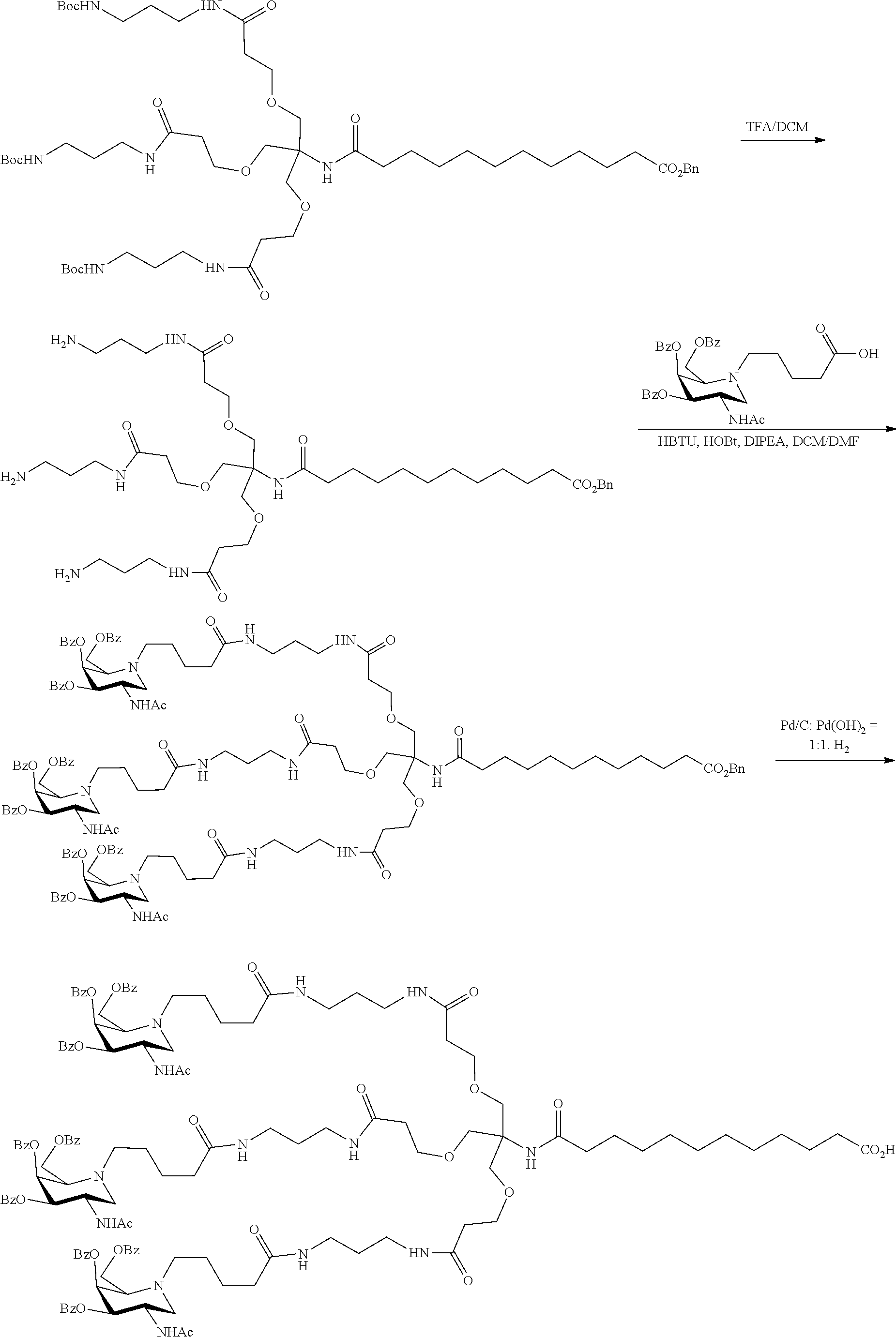

Step 1: To the solution of benzyl 15,15-bis(13,13-dimethyl-5,11-dioxo-2,12-dioxa-6,10-diazatetradecyl)-2,2-dimethyl-4,10,17-trioxo-3,13-dioxa-5 ,9,16-triazaoctacosan-28-oate (144 mg, 0.13 mmol) in DCM (2.4 mL) was added 2,2,2-trifuloroacetic acid (0.48 mL, 6.25 mmol). The reaction mixture was stirred at room temperature overnight. The solvent was evaporated under reduced pressure and crude product was co-evaporated with toluene, triturated with ether, and dried under vacuum overnight. Benzyl 12-((1,19-diamino-10-43-aminopropyl)amino)-3-oxopropoxy)methyl)-5,15-dioxo-8,12-dioxa-4,16-diazanonadecan-10-yl)amino)-12-oxododecanoate was used directly for next step without purification. LCMS calculated for C₄₁H₇₃N₇O₉ [M+H]⁺: m/z 808.56, found: 808.30.

Step 2: To the solution of 5-((2R,3S,4R,5S)-5-acetamido-3,4-bis(benzoyloxy)-2-((benzoyloxy)methyl)piperidin-1-yl)pentanoic acid (320 mg, 0.52 mmol), HATU (209 mg, 0.55 mmol) in DCM (1.5 mL) was added DIPEA (269 mg, 2.09 mmol) and crude benzyl 12-((1,19-diamino-10-43-((3-aminopropyl)amino)-3-oxopropoxy)methyl)-5,15-dioxo-8,12-dioxa-4,16-diazanonadecan-10-yl)amino)-12-oxododecanoate (0.13 mmol) in DMF (0.25 mL). The mixture was stirred at room temperature for 4 h. Solvent was evaporated under reduced pressure to give crude residue which was purified by flash chromatography (5% MeOH in DCM to 30% MeOH in DCM) to give benzyl 1-((2R,3S,4R,5S)-5-acetamido-3,4-bis(benzoyloxy)-2-((benzoyloxy)methyl)piperidin-1-yl)-16,16-bis((3-((3-(5-((2R,3 S,4R,5 S)-5-acetamido-3,4-bis (benzoyloxy)-2-((benzoyloxy)methyl)piperidin-1-yl)pentanamido)propyl)amino)-3-oxopropoxy)methyl)-5,11,18-trioxo-14-oxa-6,10,17-triazanonaco san-29-oate (212 mg, 63% yield) as white solid.

Step 3: To the solution of benzyl 1-((2R,3S,4R,5S)-5-acetamido-3,4-bis(benzoyloxy)-2-((benzoyloxy)methyl)piperidin-1-yl)-16,16-bis((3-((3-(5-((2R,3 S,4R,5 S)-5-acetamido-3,4-bis(benzoyloxy)-2-((benzoyloxy)methyl)piperidin-1-yl)pentanamido)propyl)amino)-3-oxopropoxy)methyl)-5,11,18-trioxo-14-oxa-6,10,17-triazanonacosan-29-oate (106 mg, 0.0407 mmol) in methanol: ethyl acetate (1:1, 2 mL) was added 10% Pd(OH)_{2/}C (2.9 mg, 0.0203 mmol) and Pd/C (2.6 mg, 0.0203 mmol) and purged with argon. The flask was then purged with H2 and stirred under H2 atmosphere. The reaction was stopped after the complete consumption of starting material which was confirmed by LCMS. The reaction mixture was filtered through celite to give 1-((2R,3S,4R,5S)-5-acetamido-3,4-bis(benzoyloxy)-2-((benzoyloxy)methyl)piperidin-1-yl)-16,16-bis ((3-((3-(5-((2R,3 S,4R,5 S)-5-acetamido-3,4-bis(benzoyloxy)-2-((benzoyloxy)methyl)piperidin-1-yl)pentanamido)propyl)amino)-3-oxopropoxy)methyl)-5,11,18-trioxo-14-oxa-6,10,17-triazanonacosan-29-oic acid (82 mg, 80% yield) as white solid. LCMS calculated for C₁₃₈H₁₆₉N₁₃O₃₃ [M/2+H]⁺: m/z 1257.12, found: 1257.77.

### Synthesis of 1-((2R,3 S,4R,5 S)-5-acetamido-3,4-bis (benzoyloxy)-2-((benzoyloxy)methyl)piperidin-1-yl)-16,16-bis((3-((3-(5-((2R,3 S,4R,5 S)-5-acetamido-3,4-bis(benzoyloxy)-2-((benzoyloxy)methyl)pipe ridin-1-yl)-5-oxopentanamido)propyl)amino)-3-oxopropoxy)methyl)-1,5,11,18-tetraoxo-14-oxa-6,10,17-triazanonacosan-29-oic acid (e.g., for Mod153)

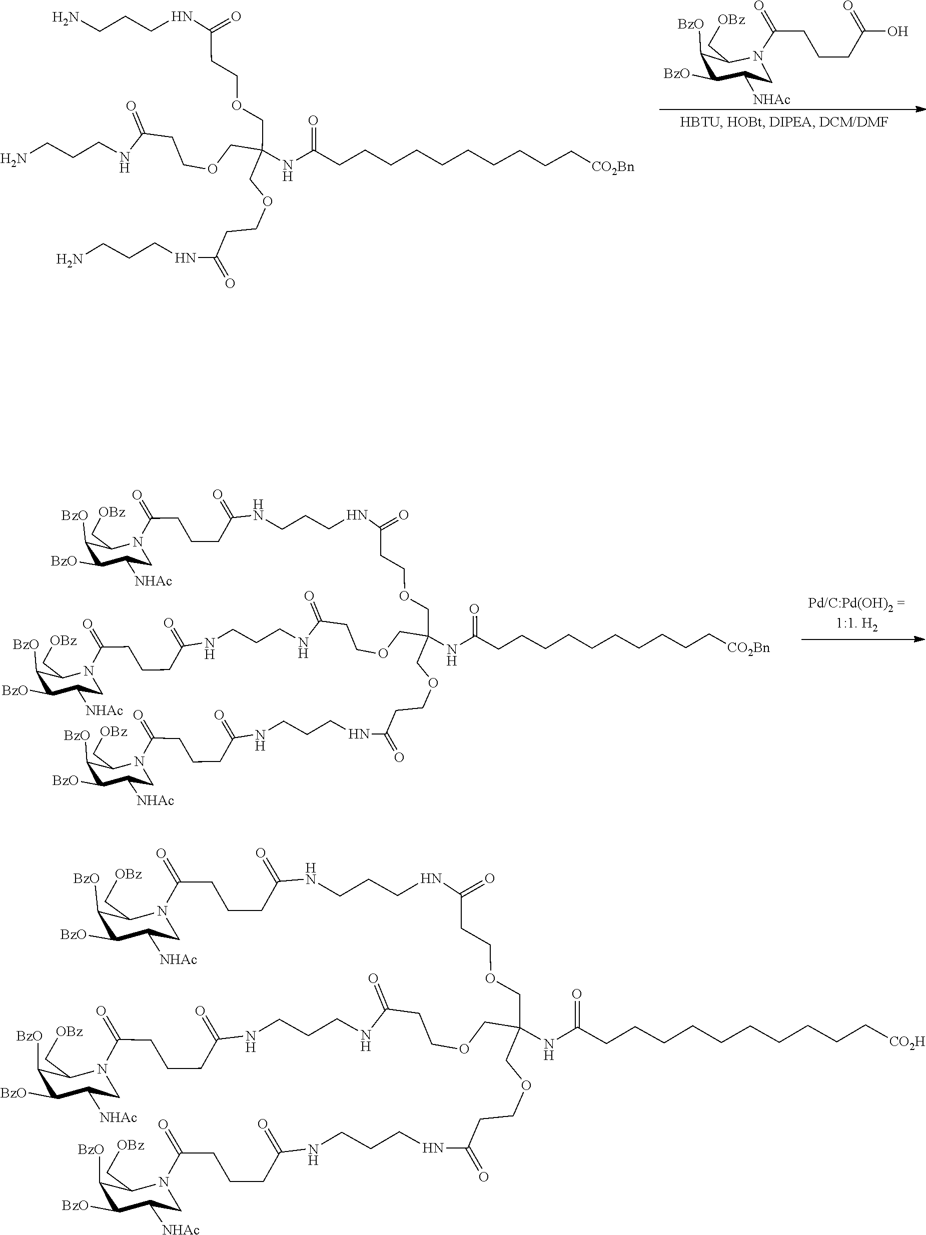

Step 1: To the solution of 5-((2R,3S,4R,5S)-5-acetamido-3,4-bis(benzoyloxy)-2-((benzoyloxy)methyl)piperidin-1-yl)-5-oxopentanoic acid (328 mg, 0.52 mmol), HATU (209 mg, 0.55 mmol) in DCM (1.5 mL) was added DIPEA (269 mg, 2.08 mmol) and benzyl 12-((1,19-diamino-10-43-((3-aminopropyl)amino)-3-oxopropoxy)methyl)-5,15-dioxo-8,12-dioxa-4,16-diazanonadecan-10-yl)amino)-12-oxododecanoate (0.13 mmol) in DMF (0.25 mL). The mixture was stirred at room temperature for 5 hrs. Solvent was evaporated under reduced pressure to give crude residue which was purified by flash chromatography (5% MeOH in DCM to 30% MeOH in DCM) to give benzyl 1-((2R,3 S,4R,5 S)-5-acetamido-3,4-bis (benzoyloxy)-2-((benzoyloxy)methyl)piperidin-1-yl)-16,16-bis Step 2: To the solution of benzyl 1-42R,3S,4R,5S)-5-acetamido-3,4-bis(benzoyloxy)-2-((benzoyloxy)methyl)piperidin-1-yl)-16,16-bis((3-((3-(5-((2R,3S,4R,5S)-5-acetamido-3,4-bis(benzoyloxy)-2-((benzoyloxy)methyl)piperidin-1-yl)-5-oxopentanamido)propyl)amino)-3-oxopropoxy)methyl)-1,5,11,18-tetraoxo-14-oxa-6,10,17-triazanonacosan-29-oate (193 mg, 0.0729 mmol) in methanol: ethyl acetate (1:1, 2 mL) was added 10% Pd(OH)_{2/}C (5.2 mg, 0.03645 mmol) and Pd/C (3.9 mg, 0.03645 mmol) and purged with argon. The flask was then purged with H2 and stirred under H₂ atmosphere. The reaction was stopped after the complete consumption of starting material which was confirmed by LCMS. The reaction mixture was filtered through celite and purified by flash chromatography (5% MeOH in DCM to 30% MeOH in DCM) to obtain 1-((2R,3S,4R,5S)-5-acetamido-3,4-bis(benzoyloxy)-2-((benzoyloxy)methyl)piperidin-1-yl)-16,16-bis ((3-((3-(5-((2R,3 S,4R,5 S)-5-acetamido-3,4-bis (benzoyloxy)-2-((benzoyloxy)methyl)piperidin-1-yl)-5-oxopentanamido)propyl)amino)-3-oxopropoxy)methyl)-1,5,11,18-tetraoxo-14-oxa-6,10,17-triazanonaco san-29-oic acid (124 mg, 67% yield) as white solid. LCMS calculated for C₁₃₆H₁₆₃N₁₃O₃₆ [M/2+H]⁺: m/z 1278.07, found: 1278.08. ((3-((3-(5-((2R,3S,4R,5S)-5-acetamido-3,4-bis(benzoyloxy)-2-((benzoyloxy)methyl)piperidin-1-yl)-5-oxopentanamido)propyl)amino)-3-oxopropoxy)methyl)-1,5,11,18-tetraoxo-14-oxa-6,10,17-triazanonacosan-29-oate (193 mg, 56% yield) as white solid. LCMS calculated for C₁₄₃H₁₆₉N₁₃O₃₆ [M/3+H]⁺: m/z 882.40, found: 882.21.

### Example 26. Certain Useful Reagents for Oligonucleotide Synthesis

In some embodiments, the present disclosure provides reagents for oligonucleotide synthesis. In some embodiments, the present disclosure provides monomers for oligonucleotide synthesis. Certain useful reagents, e.g., acyclic morpholine monomers, and their preparation are described below.

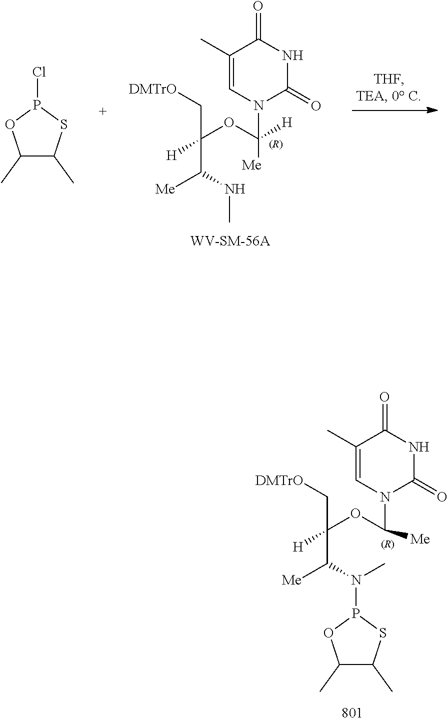

The 5′-ODMTr protected morpholino nucleoside (5.05 mmol) was dried in a three neck 100 mL round bottom flask by co-evaporating with anhydrous toluene (50 mL) followed by under high vacuum for 18 h. The dried nucleoside was dissolved in dry THF (25 mL) under argon atmosphere. Then, triethylamine (17.6 mmol, 3.5 equiv.) was added into the reaction mixture, then cooled to ˜−10° C. A THF solution of the crude chloro reagent (1.4 M solution, 1.8 equiv., 9.09 mmol) was added to the above mixture through cannula over -3 min, then, gradually warmed to room temperature over about 1 h. LCMS showed that the starting material was consumed. Then filtered carefully under vacuum/argon and the resulting filtrate was concentrated under reduced pressure to give a yellow foam which was further dried under high vacuum overnight. Crude mixture was purified by silica gel column [Column was pre-deactivated using acetonitrile then ethyl acetate (5% TEA) and then equilibrated using ethyl acetate-hexanes] chromatography using ethyl acetate and hexane as eluents. Data for 801: Yield 66%. ³¹P NMR (243 MHz, CDCl₃) δ 154.93, 154.65, 154.58, 154.23, 150.54, 150.17, 145.69, 145.26; MS (ES) m/z calculated for C₃₇H₄₆N₂O₇PS [M+K]⁺746.24, Observed: 746.38 [M+K]⁺.

### Synthesis of WV—SM-56a

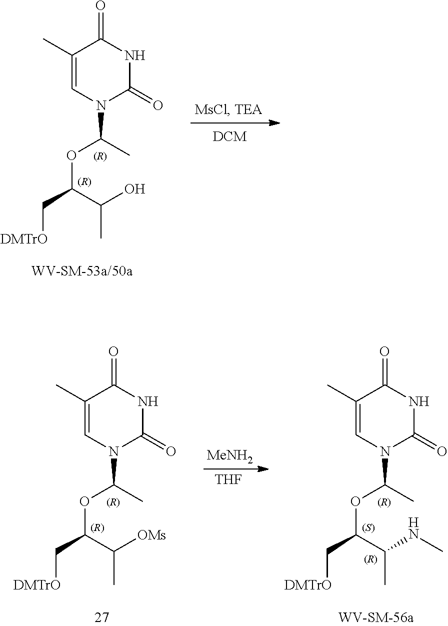

Preparation of compound 27: To a solution of WV—SM-53a/50a (6 g, 10.70 mmol) in DCM (40 mL) was added Et₃N (3.25 g, 32.11 mmol) and MsCl (2.45 g, 21.40 mmol) in DCM (20 mL) at 0° C. The mixture was stirred at 0° C. for 4 hr. TLC showed WV—SM-53a/50a was consumed and one new spot was detected. The reaction mixture was quenched by addition sat. NaHCO₃ (aq., 50 mL), and then extracted with EtOAc (50 mL * 3). The combined organic layers were washed with brine (50 mL), dried over Na₂SO₄, filtered and concentrated under reduced pressure to give a residue. Compound 27 (8.0 g, crude) was obtained as a brown oil. TLC Petroleum ether: Ethyl acetate=1:3, R_{f}=0.50.

Preparation of WV—SM-56a: Two batches: To a solution of compound 27 (3.42 g, 5.35 mmol,) in THF (20 mL) was added methanamine (10 g, 96.60 mmol, 30% purity). The mixture was stirred at 100° C. for 160 hr. LC-MS showed compound 27 was consumed and one main peak with desired MS was detected. And TLC showed one main spot. 2 batches were combined and the reaction mixture was filtered and concentrated under reduced pressure to give a residue. The residue was purified by MPLC (SiO₂, Petroleum ether/Ethyl acetate=5:1 to 0:1, 5% TEA). WV—SM-56a (2.9 g, 47.21% yield) was obtained as yellow solid. ¹H NMR (400 MHz, CHLOROFORM-d) δ=7.29-7.24 (m, 2H), 7.20-7.06 (m, 8H), 6.72 (d, J=8.8 Hz, 4H), 6.08-5.87 (m, 1H), 3.71 (s, 6H), 3.58-3.42 (m, 1H), 3.19-3.05 (m, 1H), 3.05-2.91 (m, 1H), 2.83-2.75 (m, 1H), 2.72 (d, J=4.8 Hz, 2H), 2.31 (s, 3H), 1.61 (dd, J=0.9, 5.9 Hz, 3H), 1.36 (d, J=5.9 Hz, 3H), 0.96-0.77 (m, 3H). ¹³C NMR (101 MHz, CHLOROFORM-d) δ=163.71, 163.62, 158.47, 150.74, 150.58, 144.72, 135.94, 135.89, 135.86, 135.25, 135.15, 130.02, 129.93, 129.89, 127.90 (dd, J=2.9, 22.0 Hz, 1C), 126.83, 126.81, 113.10, 113.08, 111.28, 111.24, 86.45, 86.39, 81.89, 81.82, 81.00, 80.58, 63.39, 63.15, 60.40, 56.02, 55.23, 34.52, 34.17, 26.41, 23.11, 21.66, 21.59, 15.57, 15.09, 14.20, 12.46, 12.41. HPLC purity: 90.87%. LCMS (M+Na⁺): 596.3. SFC: dr=52.46: 47.54. TLC (ethyl acetate: methanol=9:1), R_{f}=0.19.

### Synthesis of WV—SM-53a and WV—SM-50a

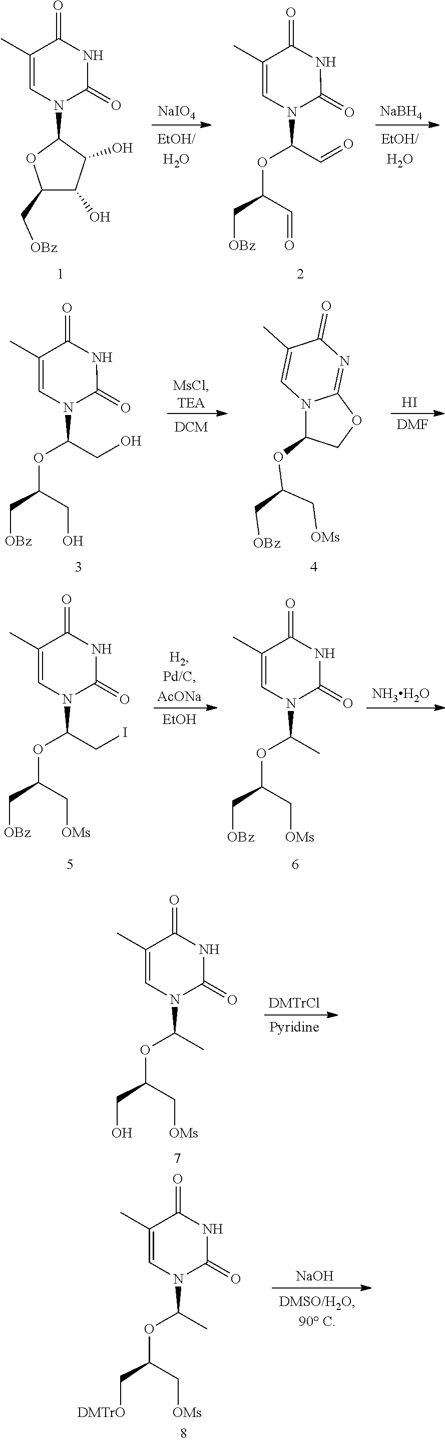

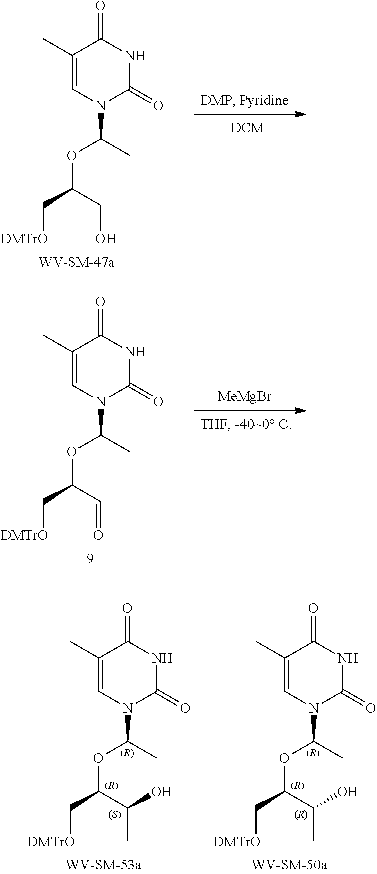

Preparation from Compound 1: 2 batches: To a solution of compound 1 (50 g, 137.99 mmol) in EtOH (1000 mL) was added NaIO₄ (30.00 g, 140.26 mmol) in H₂O (500 mL). The mixture was stirred in dark at 15° C. for 2 hr. TLC indicated compound 1 was consumed and one new spot formed. Compound 2 (99.44 g, crude) was obtained as a white suspension liquid, which was used next step. TLC (Ethyl acetate: Methanol=9:1), R_{f}=0.49.

Preparation of Compound 3: 2 batches: To a stirred solution of compound 2 (49.72 g, 137.99 mmol) in EtOH (1000 mL) and H₂O (500 mL) was added NaBH₄ (10.44 g, 275.98 mmol) in small portions at 0° C. The mixture was stirred at 15° C. for 1 hr. TLC indicated compound 2 was consumed and one new spot formed. 1N HCl was added to pH=7. The solvent was removed to yield a brown solid. The solid was added sat. Na₂SO₃ (aq., 500 mL) and then extracted with EtOAc (500 mL*8). The combined organic phase was dried by Na₂SO₄. Removal of the solvent under reduced pressure gave the product. Compound 3 (86.7 g, 86.22% yield) was obtained as a white solid. LCMS (M+Na⁺) 386.9, purity 96.31%. TLC (Ethyl acetate: Methanol=9:1), R_{f}=0.38.

Preparation of compound 4: To a solution of compound 3 (86.7 g, 237.96 mmol) and TEA (120.40 g, 1.19 mol) in DCM (700 mL) was added MsCl (59.97 g, 523.51 mmol) in DCM (300 mL). The mixture was stirred at 0° C. for 4 hr. TLC indicated compound 3 was consumed, and two new spots formed. The reaction mixture was quenched by addition water (500 mL) and stayed for 36 hr. TLC indicated the intermediate was consumed, and one spot left. The water layer was extracted with DCM (800 mL * 3). The combined organic layers were dried over Na₂SO₄, filtered and concentrated under reduced pressure to give a residue. The residue was purified by column chromatography (SiO₂, Petroleum ether/Ethyl acetate=20/1 to 0:1 and then MeOH/EtOAc=0/1 to 1/10). Compound 4 (75 g, 74.26% yield) was obtained as a white solid. TLC (Petroleum ether: Ethyl acetate=0: 1), R_{f}=0.38; (Ethyl acetate: Methanol=9: 1), R_{f}=0.13.

Preparation of compound 5: To a solution of compound 4 (75 g, 176.71 mmol) in DMF (650 mL) was added HI (100.46 g, 353.42 mmol, 59.09 mL, 45% purity). The mixture was stirred at 15° C. for 0.5 hr. TLC showed compound 4 was consumed and one main spot was detected. The reaction mixture was quenched by sat. NaHCO3 (aq.) to pH=7. The residue was extracted with EtOAc (800 mL * 5). The combined organic layers were washed with brine (600 mL), dried over Na₂SO₄, filtered and concentrated under reduced pressure to give a residue. Compound 5 (91.15 g, crude) was obtained as a brown oil. TLC (Ethyl acetate: Methanol=9:1), R_{f}=0.80.

Preparation of compound 6: A mixture of compound 5 (91 g, 164.75 mmol), Pd/C (28 g, 10% purity) and NaOAc (122.85 g, 1.50 mol) in EtOH (700 mL) was degassed and purged with H₂ for 3 times, and then the mixture was stirred at 15° C. for 24 hr under H₂ atmosphere (15 psi). TLC showed compound 5 was consumed and one main spot was found. The Pd/C was filtered off and the filtrate evaporated. The residue was added with water (500 mL), extracted with EtOAc (500 mL*6). And then the organic layer was washed with brine (500 mL) and dried over Na₂SO₄, filtered and concentrated under reduced pressure to give a residue. Compound 6 (76 g, crude) was obtained as a brown oil. TLC (Petroleum ether: Ethyl acetate=1:3), R_{f}=0.12.

Preparation of compound 7: To a solution of compound 6 (70 g, 164.15 mmol) in MeOH (1000 mL) was added NH₃·H₂O (1.15 kg, 8.21 mol, 1.26 L, 25% purity). The mixture was stirred at 15° C. for 16 hr. TLC indicated compound 6 was consumed and one new spot formed. The reaction mixture was concentrated under reduced pressure to remove MeOH and the water phase was extracted with EtOAc (300 mL*8). The organic phase was dried with Na₂SO₄, filtered and concentrated under reduced pressure to give a residue. The residue was purified by column chromatography (SiO₂, Petroleum ether/Ethyl acetate=20/1 to 0:1). Compound 7 (33 g, 62.37% yield) was obtained as a white solid. TLC (Ethyl acetate: Methanol=9:1), R_{f}=0.39.

Preparation of compound 8: To a solution of compound 7 (33 g, 102.38 mmol) in pyridine (120 mL) was added DMTC1 (41.63 g, 122.85 mmol). The mixture was stirred at 15° C. for 4 hr. TLC indicated compound 7 was consumed and one new spot formed. The reaction mixture was diluted with sat. NaHCO₃ (aq., 100 mL) and extracted with EtOAc (200 mL * 5). The combined organic layers were dried over Na₂SO₄, filtered and concentrated under reduced pressure to give a residue. The residue was purified by column chromatography (SiO₂, Petroleum ether/Ethyl acetate=20/1 to 1/5, 5% TEA). Compound 8 (55 g, 86.00% yield) was obtained as a yellow solid. TLC (Petroleum ether: Ethyl acetate=0:1), R_{f}=0.65.

Preparation of WV—SM-47a: A mixture of compound 8 (55 g, 88.04 mmol) , NaOH (42.26 g, 1.06 mol) in DMSO (300 mL) and Water (300 mL) was degassed and purged with N₂ for 3 times, and then the mixture was stirred at 90° C. for 16 hr under N₂ atmosphere. LCMS and TLC showed the compound 8 was completed, and one main peak with desired MS 545 (NEG, M−H⁺) was found. The reaction mixture was quenched by addition EtOAc (1000 mL), and then diluted with H₂O (1000 mL) and extracted with EtOAc (1000 mL * 4). The combined organic layers were washed with brine (1000 mL), dried over Na₂SO₄, filtered and concentrated under reduced pressure to give a residue. The residue was purified by column chromatography (SiO₂, Petroleum ether/Ethyl acetate=20/1 to 1/3, 5% TEA). WV—SM-47a (37.5 g, 77.92% yield) was obtained as a white solid. LCMS (M-Ft) 545.3. TLC (Petroleum ether: Ethyl acetate=0:1, 5% TEA), R_{f}=0.29.

Preparation of compound 9: To a solution of WV—SM-47a (37.5 g, 68.60 mmol) in DCM (400 mL) was added pyridine (81.40 g, 1.03 mol, 83.06 mL) and Dess-Martin periodinane (34.92 g, 82.33 mmol). The mixture was stirred at 20° C. for 4 hr. LC-MS showed WV—SM-47a was consumed completely and new peak with desired MS was detected. The reaction mixture was quenched by addition sat. NaHCO₃ (aq., 1000 mL) and sat. Na₂SO₃ (aq.) 1000 mL, and then extracted with EtOAc (100 mL * 5). The combined organic layers were washed with brine 500 mL, dried over Na₂SO₄, filtered and concentrated under reduced pressure to give a residue. Compound 9 (43 g, crude) was obtained as a yellow solid. LCMS (M−H⁺) 543.3.

Preparation of WV-NU-53a and WV-NU-50a: To a solution of compound 9 (37.36 g, 68.60 mmol) in THF (300 mL) was added MeMgBr (3 M, 68.60 mL) at −40° C. The mixture was stirred at −40-15° C. for 6 hr. LC-MS showed compound 9 was consumed completely and new peaks with mass was detected. The reaction mixture was quenched by addition water (20 mL) at 0° C., and then extracted with EtOAc (300 mL * 3). The combined organic layers were washed with brine (200 mL), dried over Na₂SO₄, filtered and concentrated under reduced pressure to give a residue. TLC showed one main spot. The residue was purified by column chromatography (SiO₂, Petroleum ether/Ethyl acetate=20/1 to 0/1, 5% TEA). 6 g of the residue was purified by SFC (column: DAICEL CHIRALPAK AD-H(250 mm*30 mm, 5 um);mobile phase: [0.1%NH₃H₂O IPA];B%: 39%-39%,9.33min). And crude WV—SM-50a was purified by prep-HPLC (column: Agela Durashell 10u 250*50 mm;mobile phase: [water(0.04%NH₃H₂O)-ACN];B%: 37%-56%,20min). WV—SM-53a (1.4 g, 23.33% yield) was obtained as a white solid. WV—SM-50a (1.8 g, 30.00% yield) was obtained as a white solid. 0.5 g of WV—SM-53a: ¹H NMR (400 MHz, CHLOROFORM-d) δ=7.37-7.30 (m, 2H), 7.28-7.18 (m, 8H), 7.12 (d, J=1.1 Hz, 1H), 6.80 (d, J=8.6 Hz, 4H), 6.08 (q, J=5.8 Hz, 1H), 4.09-3.99 (m, 1H), 3.79 (d, J=0.9 Hz, 6H), 3.51 (q, J=5.0 Hz, 1H), 3.20-3.05 (m, 2H), 2.70 (q, J=7.1 Hz, 2H), 1.71 (d, J=1.1 Hz, 3H), 1.46 (d, J=6.0 Hz, 3H), 1.14-1.10 (m, 3H). ¹³C NMR (101 MHz, CHLOROFORM-d) δ=163.19, 158.54, 150.48, 144.39, 135.53, 134.91, 129.86, 129.81, 127.90, 127.86, 126.93, 113.15, 111.48, 86.73, 81.44, 81.24, 68.14, 63.45, 55.22, 45.74, 21.45, 18.01, 12.43. HPLC purity: 99.04%. LCMS (M−H⁺): 559.0. SFC dr=99.83: 0.17. TLC (Petroleum ether: Ethyl acetate=1:3), R_{f}=0.28. 0.9 g of WV—SM-53a: ¹FINMR (400 MHz, CHLOROFORM-d) δ=7.36-7.30 (m, 2H), 7.29-7.15 (m, 9H), 7.13 (s, 1H), 6.80 (d, J=8.8 Hz, 4H), 6.08 (q, J=6.0 Hz, 1H), 4.11-3.97 (m, 1H), 3.79 (s, 6H), 3.51 (q, J=4.9 Hz, 1H), 3.13 (dq, J=5.3, 10.1 Hz, 2H), 1.72 (s, 3H), 1.47 (d, J=6.2 Hz, 3H), 1.10 (d, J=6.4 Hz, 3H). ¹³C NMR (101 MHz, CHLOROFORM-d) δ=163.19, 158.54, 150.47, 144.39, 135.50, 134.92, 129.86, 129.81, 127.89, 127.87, 126.94, 113.15, 111.48, 86.73, 81.44, 81.25, 68.14, 63.45, 55.22, 45.19, 21.46, 18.02, 12.44. HPLC purity: 97.56%. LCMS (M−H⁺): 559.1, purity 92.9%. SFC dr=98.49: 1.51. 1.75 g of WV—SM-50a: ¹FINMR (400 MHz, CHLOROFORM-d) δ=8.41 (s, 1H), 7.35-7.31 (m, 2H), 7.26-7.19 (m, 7H), 7.11 (d, J=1.3 Hz, 1H), 6.82-6.77 (m, 4H), 6.00 (q, J=5.7 Hz, 1H), 4.09-4.00 (m, 1H), 3.79 (d, J=0.9 Hz, 6H), 3.51-3.44 (m, 1H), 3.22 (dd, J=5.3, 10.1 Hz, 1H), 3.02 (dd, J=5.3, 10.1 Hz, 1H), 2.20 (br s, 1H), 1.72 (d, J=0.9 Hz, 3H), 1.47 (d, J=6.1 Hz, 3H), 1.17 (d, J=6.6 Hz, 3H). ¹³C NMR (101 MHz, CHLOROFORM-d) δ=163.29, 158.50, 150.43, 144.40, 135.55, 135.45, 134.86, 129.88, 129.84, 127.93, 127.84, 126.94, 113.12, 111.46, 86.55, 82.48, 82.43, 67.59, 63.24, 55.22, 21.40, 19.17, 12.43. HPLC purity: 96.51%. LCMS (M−H⁺): 559.2, purity 93.04%. SFC dr=0.88: 99.12.

### Example 27. Certain Useful Technologies for Preparing Oligonucleotide Compositions

As described herein, various technologies can be utilized to prepare oligonucleotide compositions in accordance with the present disclosure. Certain useful technologies are described in certain useful chirally controlled preparation technologies, including oligonucleotide synthesis cycles, reagents and conditions are described in U.S. Pat. Nos. 9,394,333, 9,744,183, 9,605,019, 9,598,458, 9,982,257, 10,160,969, 10,479,995, US 2020/0056173, US 2018/0216107, US 2019/0127733, U.S. Pat. No. 10,450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the oligonucleotide synthesis technologies of each of which are independently incorporated herein by reference.

A useful procedure for preparing oligonucleotide compositions including chirally controlled oligonucleotide compositions (25 μmol scale) is described below as an example.

Automated solid-phase preparation of oligonucleotide compositions including chirally controlled oligonucleotide compositions was performed according to the cycles shown in the tables below: regular amidite cycle can be utilized for PO linkages; DPSE amidite cycle can be utilized for, e.g., chirally controlled phosphorothioate linkages; and MBR/MMPC amidite cycle can be utilized for, e.g., stereorandom or chirally controlled morpholino PN linkages (e.g., n001); and regular amidite cycle can be utilized for, e.g., sterorandom PN linkages (e.g., n001); and PSM amidite cycle for, e.g., chirally controlled PN linkages (e.g., n001).

Regular Amidite Synthetic Cycle for PO linkages.

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | oxidation | 50 mM I₂/pyridine-H₂O (9:1, v/v) | 2.0 mL | 1 min |
| 4 | cap-2 | 20% Ac₂O, 30% 2,6-lutidine/MeCN | 1.0 mL | 45 s |
| | | 20% MeIm/MeCN | 1.0 mL | |

### DPSE Amidite Synthetic Cycle for Chirally Controlled PS (Phosphorothioate) Linkages

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | cap-1 | 20% Ac₂O, 30% 2,6-lutidine/MeCN | 2.0 mL | 2 min |
| 4 | sulfurization | 0.2M XH/pyridine | 2.0 mL | 6 min |
| 5 | cap-2 | 20% Ac₂O, 30% 2,6-lutidine/MeCN | 1.0 mL | 45 s |
| | | 20% MeIm/MeCN | 1.0 mL | |

MBR/MMPC Amidite Synthetic Cycle (P(V)) for Stereorandom/Chirally Controlled PN Linkages

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 10 min |
| | | 1.0M DBU/MeCN | 1.0 mL | |
| 3 | cap-2 | 20% Ac₂O, 30% 2,6-lutidine/MeCN | 1.0 mL | 45 s |
| | | 20% MeIm/MeCN | 1.0 mL | |

Regular Amidite Synthetic Cycle for Stereorandom PN Linkages

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | imidation | 0.5M ADIH/MeCN | 2.0 mL | 6 min |
| 4 | cap-2 | 20% Ac₂O, 30% 2,6-lutidine/MeCN | 1.0 mL | 45 s |
| | | 20% MeIm/MeCN | 1.0 mL | |

PSM Amidite Synthetic Cycle for Chirally Controlled PN Linkages

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | cap-1 | 20% Ac₂O, 30% 2,6-lutidine/MeCN | 2.0 mL | 2 min |
| 4 | imidation | 0.5M azide reagent/MeCN | 2.0 mL | 6 min |
| 5 | cap-2 | 20% Ac₂O, 30% 2,6-lutidine/MeCN | 1.0 mL | 45 s |
| | | 20% MeIm/MeCN | 1.0 mL | |

In some embodiments, the following procedure was used for C&D (25 μmol scale): After completion of the synthesis, the CPG solid support was dried and transferred into 50 mL plastic tube. The CPG was treated with 1× reagent (2.5 mL; 100 μL/umol) for 3 h at 28° C., then added conc. NH₃ (aqueous solution, 5.0 mL; 200 μL/umol) for 16 h at 45° C. The reaction mixture was cooled to room temperature and the CPG was separated by membrane filtration, washed with 15 mL of H₂O. The crude material (filtrate) was analyzed by LTQ and RP-UPLC.

In some embodiments, the following procedure was used for GalNAc conjugation conditions (1 μmol scale): Into a plastic tube, tri-GalNAc (2.0 eq.), HATU (1.9 eq.), and DIPEA (10 eq.) were dissolved in anhydrous MeCN (0.5 mL). The mixture was stirred for 10 min at room temperature, then the mixture was added into the amino-oligo (1 μmol) in H₂O (1 mL) and stirred for 1 h at 37° C. The reaction was monitored by LC-MS and RP-UPLC. After the reaction was completed, the resultant GalNAc-conjugated oligo was treated with conc. NH₃ (aqueous solution, 2 mL) for 1 h at 37° C. The solution was concentrated under vacuum to remove MeCN and conc. NH₃ aqueous solution. The residue was then dissolved in H₂O (10 mL) for reversed phase purification.

Various oligonucleotide compositions, e.g., various compositions in Table A1, A2, A3 and A4, were prepared. Certain MS data are presented below as examples:

| ID | calcd. [M] | observed [M] |
|---|---|---|
| WV-44364 | 7016.8 | 7020.5 |
| WV-44365 | 7145.8 | 7149.5 |
| WV-44366 | 6914.7 | 6918.7 |
| WV-40387 | 6890.8 | 6892.7 |
| WV-44367 | 7065.0 | 7069.0 |
| WV-44368 | 7194.0 | 7198.0 |
| WV-44369 | 6962.9 | 6965.8 |
| WV-40386 | 7144.0 | 7145.2 |
| WV-44359 | 7318.2 | 7321.4 |
| WV-44360 | 7447.3 | 7450.6 |
| WV-44361 | 7216.2 | 7219.1 |
| WV-8587 | 6813.4 | 6812.8 |
| WV-15562 | 7098.9 | 7099.1 |
| WV-43245 | 7119.0 | 7117.1 |
| WV-43246 | 6992.7 | 6992.7 |
| WV-43247 | 7048.8 | 7048.8 |
| WV-43248 | 7261.1 | 7260.6 |
| WV-43249 | 7140.9 | 7140.6 |
| WV-43250 | 7183.0 | 7182.6 |
| WV-43251 | 7225.1 | 7224.9 |
| WV-43252 | 7309.3 | 7309.7 |
| WV-43253 | 7351.3 | 7351.0 |
| WV-43254 | 7519.7 | 7519.3 |
| WV-43255 | 7309.3 | 7308.8 |
| WV-22749 | 6711.9 | 6711.3 |
| WV-21218 | 6949.1 | 6949.6 |
| WV-43239 | 6991.2 | 6988.8 |
| WV-43240 | 7033.3 | 7031.1 |
| WV-43241 | 7075.4 | 7072.9 |
| WV-43242 | 7159.5 | 7159.2 |
| WV-43243 | 7201.6 | 7200.2 |
| WV-43244 | 7369.9 | 7369.2 |
| WV-43287 | 7111.4 | 7109.0 |
| WV-15885 | 7270.0 | 7272.2 |
| WV-15887 | 7270.0 | 7272.5 |
| WV-24104 | 7399.1 | 7399.1 |
| WV-24105 | 7399.1 | 7399.8 |
| WV-41422 | 7168.0 | 7170.8 |
| WV-41430 | 7168.0 | 7170.7 |
| WV-44357 | 7104.9 | 7103.0 |
| WV-44370 | 7354.3 | 7351.9 |
| WV-45083 | 7183.0 | 7182.9 |
| WV-45084 | 7267.2 | 7267.5 |
| WV-45085 | 7267.2 | 7268.1 |
| WV-45086 | 7363.2 | 7364.4 |
| WV-45087 | 7423.4 | 7424.9 |
| WV-45088 | 7423.4 | 7426.0 |
| WV-38642 | 7082.8 | 7083.9 |
| WV-44962 | 7098.9 | 7099.2 |
| WV-44964 | 6894.6 | 6896.6 |
| WV-44966 | 6813.4 | 6813.6 |
| WV-44468 | 6908.6 | 6909.8 |
| WV-44469 | 6993.7 | 6994.0 |
| WV-44470 | 7297.0 | 7297.9 |
| WV-45140 | 8603.6 | 8605.3 |
| WV-8587 | 6813.4 | 6812.8 |
| WV-40579 | 6892.5 | 6891.3 |
| WV-40831 | 6892.5 | 6891.1 |
| WV-40832 | 6892.5 | 6891.4 |
| WV-40807 | 6756.4 | 6757.5 |
| WV-40808 | 6796.4 | 6797.3 |
| WV-40835 | 6779.5 | 6781.6 |
| WV-30938 | 7082.8 | 7082.3 |
| WV-39402 | 6946.7 | 6947.2 |
| WV-39291 | 6986.7 | 6987.6 |

In some embodiments, internucleotidic linkages were constructed using reagents, conditions, e.g., as described below:

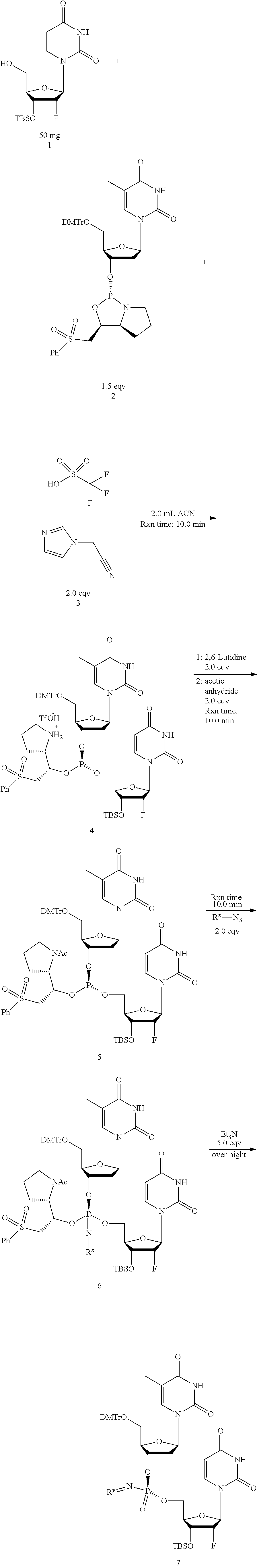

A useful procedure: To a dry 20 mL vial was added amidite 2 (1.5 eq.) and dissolved in anhydrous acetonitrile (1 mL) under argon. To the reaction mixture was added CMMIT 3 (2.0 eq) followed by addition of alcohol 1 (50 mg, 1.5 eq) and reaction was stirred at room temperature. Progress of the reaction was monitored by LCMS. After disappearance of alcohol, to the reaction mixture was added 2,6-lutidine (2.0 eq) and acetic anhydride (2.0 eq). After 10 min stirring at room temperature, to the reaction mixture was added azide (2.0 eq) dissolved in 0.5 mL anhydrous acetonitrile. The progress of reaction was monitored by LCMS. After completion of reaction (reaction time 10 min), triethylamine (5.0 eq) was added and reaction mixture was stirred at room temperature for 5 hrs to 16 hrs to give final product (7). In some embodiments, P^{N} is P═N—R^{x}. In some embodiments, —X—R^{L} is —N═R^{y}.

| | LC-MS | | LC-MS |
|---|---|---|---|
| | (observed) | | (observed) |
| R^{y} = N— (7) | (M − H)⁻ | R = N— (7) | (M − H)⁻ |
| 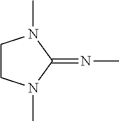 | 1061.21 | 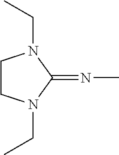 | 1088.79 |
| 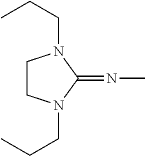 | 1116.65 | 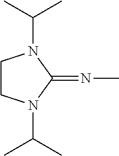 | 1116.65 |
| 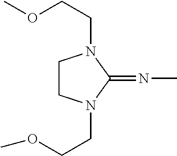 | 1149.06 | 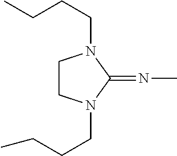 | 1145.08 |
| 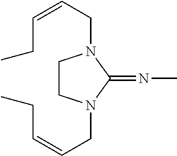 [SMILES: CC/C=C\CN1CCN(C/C=C\CC)C1=NC] | 1168.87 | 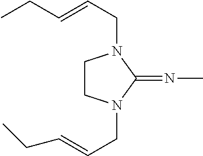 [SMILES: CC/C=C/CN1CCN(C/C=C/CC)C1=NC] | 1168.87 |
| 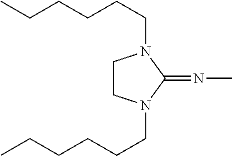 [SMILES: CCCCCCN1CCN(CCCCCC)C1=NC] | 1201.00 | 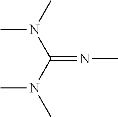 | 1063.21 |
| 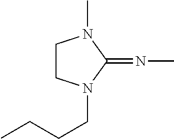 | 1102.81 | 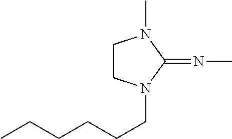 | 1132.31 |
| 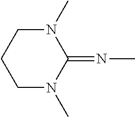 | 1074.86 | 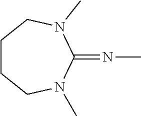 | 1088.79 |
| 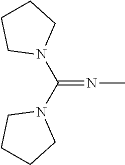 | 1115.32 | 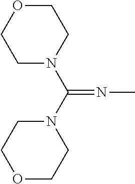 | 1148.36 |
| 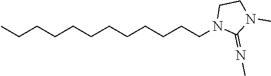 [SMILES: CCCCCCCCCCCCN1CCN(C)C1=NC] | 1214.48 | 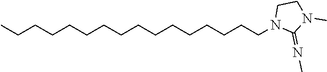 [SMILES: CCCCCCCCCCCCCCCCN1CCN(C)C1=NC] | 1271.57 |
| 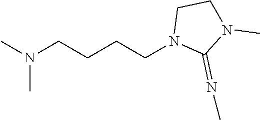 | 1146.64 | 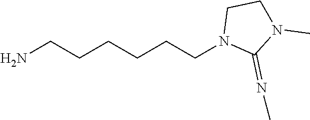 | 1146.53 |
| 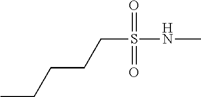 | 1099.43 | 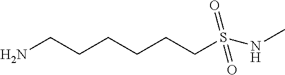 | 1128.32 |
| 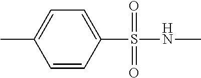 | 1119.32 | 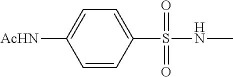 | 1162.31 |
| 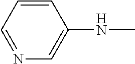 | 1041.69 | 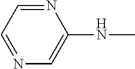 | 1043.21 |
| 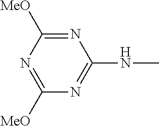 | 1103.86 | 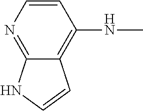 | 1082.73 |
| 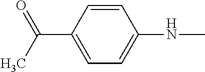 | 1082.72 | 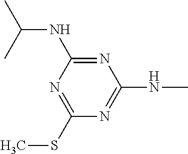 | 1187.06 |
| 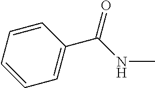 | 1068.21 | 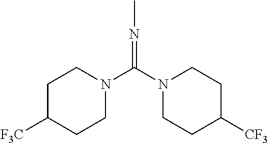 [SMILES: CN=C(N1CCC(C(F)(F)F)CC1)N1CCC(C(F)(F)F)CC1] | 1295.32 |

Various dimers, including those prepared using solid phase synthesis, were characterized and confirmed using, e.g., LC, NMR, MS, Crystallography, etc. (e.g., fCn001RfC, fCn001SfC). Using certain dimers, e.g., Geon001mU, m5Ceon001mU, mGn001mU, mUn001mU, etc., it was confirmed that provided technologies can provide very high stereoselectivity (e.g., in various embodiments when using L- and D- PSM, providing 99% or more Rp or Sp).

Certain data for oligonucleotide and compositions thereof were provided below:

| ID | Calculated MW (Da) | Observed MW (Da) |
|---|---|---|
| WV-8587 | 6813.4 | 6814.0 |
| WV-11533 | 7098.9 | 7099.7 |
| WV-13303 | 7050.7 | 7050.7 |
| WV-13304 | 7336.1 | 7337.3 |
| WV-15562 | 7098.9 | 7097.8 |
| WV-15563 | 7098.9 | 7099.7 |
| WV-24104 | 7402.1 | 7399.1 |
| WV-24109 | 7357.1 | 7355.1 |
| WV-30915 | 7082.8 | 7084.2 |
| WV-30916 | 7082.8 | 7084.9 |
| WV-38634 | 7082.8 | 7085.1 |
| WV-38635 | 7082.8 | 7084.5 |
| WV-38636 | 7161.9 | 7164.3 |
| WV-38637 | 7161.9 | 7163.8 |
| WV-38638 | 7161.9 | 7163.1 |
| WV-43248 | 7261.1 | 7260.6 |
| WV-43249 | 7140.9 | 7140.6 |
| WV-43250 | 7183.0 | 7182.6 |

### Example 28. Provided Technologies Can Provide High Activities

Among other things, provided technologies can provide high activities and/or various desired properties. Many technologies can be utilized to assess provided technologies in accordance with the present disclosure, e.g., in vitro assays, in vivo assays, biochemical assays, cell-based assays, animal models, clinical trials, etc. In some embodiments, oligonucleotides and compositions are assessed by a procedure described below:

An in vitro assay was used to measure knockdown of human MALAT1 mRNA transcript relative to a human HPRT ‘housekeeper’ gene by using antisense oligonucleotides targeting hMALAT1. Human iCell GABA neurons (CDI) are a >95% pure population of cerebral cortical cells derived from induced pluripotent stem cells (iPS) cells. One day before treatment with oligonucleotide to MALAT1, 96 well tissue culture plates were coated with Matrigel and cells were thawed and plated at ˜35,000 cells/well density in complete media. Oligonucleotides were diluted to 10X final treatment concentration in water. On the day of treatment, overnight media was removed, and 180uL fresh media added. Oligonucleotides were added in 20uL volume. Final oligonucleotide concentration ranged from 20 uM -1 pM for dose response experiments and 1, 0.2 and 0.04 uM for 3 point dosing experiments. Four days post-treatment, media was removed, and cells were lysed using Trizol. RNA was extracted using Qiagen 96 well RNA purification plate. Samples were treated with DNAse on the Qiagen RNA purification column. Alternatively, the Promega SV96 Total RNA Isolation kit was also used for RNA extraction. RNA was reverse transcribed using High Capacity cDNA Reverse Transcription kit from Applied Biosystems. Quantitative PCR was performed on Biorad CFX384 Touch Real Time system. Probes to detect human MALAT1 were from Thermo Fisher (Hs00273907_s1, FAM-MGB dye). Human HPRT1 transcript (Hs02800695_ml, VIC-MGB_PL) or Human SRSF9 was used as a normalizer (Forward 5′ TGGAATATGCCCTGCGTAAA 3′, Reverse 5′ TGGTGCTTCTCTCAGGATAAAC, Probe 5′/5HEX/TG GAT GAC A/Zen/C CAA ATT CCG CTC TCA/3IABkFQ/3′. Data was processed and analyzed using CFX Manager 3.1 and the Knime qPCR workflow or calculated and analyzed with GraphPad PRISM8.

Certain assessment results are provided in the Figures as examples. Certain results were presented below.

TABLE 7
Certain IC50 results. GABA neurons, Malat1, dose response 10 uM-128 pM

| | IC50 (uM) |
|---|---|
| WV-8584 | 2.821 |
| WV-8587 | 0.08695 |
| WV-11533 | 0.01096 |
| WV-28299 | 0.0297 |
| WV-28468 | 0.02464 |
| WV-28469 | 0.02426 |
| WV-28470 | 0.01418 |

TABLE 8
Reduction of MALAT1 transcripts. In vitro, in GABA neurons. 4 day treatment.

| | % Remain | % Remain @ | % Remain |
|---|---|---|---|
| ID | @ 1 uM | 0.2 uM | @ 0.04 uM |
| WV-9491 | 108.90 | 105.37 | 153.60 |
| WV-9491 | 101.03 | 107.67 | 96.43 |
| WV-31104 | 67.54 | 90.84 | 122.91 |
| WV-31103 | 63.32 | 90.63 | 122.55 |
| WV-31071 | 72.83 | 86.91 | 110.11 |
| WV-31063 | 55.62 | 85.06 | 111.97 |
| WV-31070 | 50.34 | 85.28 | 100.98 |
| WV-31106 | 54.55 | 79.17 | 100.25 |
| WV-31073 | 48.60 | 78.41 | 89.23 |
| WV-31095 | 36.37 | 80.28 | 101.60 |
| WV-31064 | 45.48 | 71.94 | 87.61 |
| WV-31062 | 36.52 | 60.86 | 102.98 |
| WV-31060 | 30.53 | 74.06 | 93.98 |
| WV-31097 | 28.23 | 57.22 | 91.00 |
| WV-31096 | 24.76 | 53.60 | 98.35 |
| WV-31055 | 25.63 | 56.93 | 85.91 |
| WV-31107 | 21.74 | 56.67 | 100.11 |
| WV-31056 | 25.61 | 55.91 | 82.75 |
| WV-31059 | 19.67 | 53.90 | 87.86 |
| WV-31102 | 17.74 | 53.24 | 92.23 |
| WV-31099 | 22.15 | 45.77 | 81.55 |
| WV-8587 | 15.66 | 57.29 | 84.18 |
| WV-31057 | 16.38 | 47.47 | 92.24 |
| WV-31098 | 16.76 | 43.42 | 84.77 |
| WV-31108 | 16.01 | 46.69 | 81.89 |
| WV-8587 | 15.25 | 43.97 | 89.19 |
| WV-31066 | 16.34 | 48.60 | 74.95 |
| WV-31058 | 12.76 | 42.91 | 89.16 |
| WV-31061 | 14.06 | 37.80 | 89.19 |
| WV-31065 | 9.67 | 39.60 | 67.57 |
| WV-31069 | 9.97 | 35.22 | 71.30 |
| WV-31100 | 10.27 | 33.37 | 69.52 |
| WV-28299 | 8.72 | 28.15 | 73.68 |
| WV-31067 | 8.56 | 33.13 | 59.82 |
| WV-28468 | 9.86 | 25.89 | 61.28 |
| WV-28469 | 8.60 | 24.33 | 60.83 |
| WV-31105 | 4.31 | 20.69 | 59.34 |
| WV-31072 | 4.74 | 17.25 | 45.02 |
| WV-13303 | 4.73 | 14.86 | 50.51 |
| WV-28470 | 3.84 | 18.02 | 48.29 |
| WV-11533 | 2.14 | 10.38 | 42.92 |
| WV-31101 | 2.11 | 9.03 | 30.79 |
| WV-13304 | 2.02 | 7.33 | 32.70 |
| WV-31068 | 1.93 | 7.75 | 29.04 |

In vitro, in GABA neurons. 4 day treatment. Table 9 shows %IC50 of knocking down MALAT1 mRNA in iCell Neurons, dose response [10 uM-1.0 pM, 5-fold dilution]

TABLE 9
Reduction of MALAT1 transcripts.

| ID | IC50 [nM] |
|---|---|
| WV-8587 | 53.4 |
| WV-11533 | 60.6 |
| WV-15562 | 5.6 |
| WV-15563 | 6.1 |
| WV-43083 | 26.0 |
| WV-43084 | 17.3 |
| WV-40386 | 14.7 |
| WV-28299 | 8.4 |

In vitro, in GABA neurons. 4 day treatment. Table 10 shows %IC50 of knocking down MALAT1 mRNA in iCell Neurons, dose response [10 uM-25.6 pM, 5-fold dilution]

TABLE 10
Reduction of MALAT1 transcripts.

| ID | IC50 [nM] |
|---|---|
| WV-8587 | 84.1 |
| WV-11533 | 17.5 |
| WV-15562 | 7.7 |
| WV-40557 | 64.9 |
| WV-40558 | 306.9 |
| WV-40559 | 83.8 |
| WV-40560 | 120.3 |
| WV-40561 | 983.0 |
| WV-40562 | 24.5 |
| WV-40563 | 31.1 |

TABLE 11
Reduction of MALAT1 transcripts.

| ID | IC50 [nM] |
|---|---|
| WV-8587 | 247.1 |
| WV-11533 | 27.7 |
| WV-15562 | 32.4 |
| WV-15563 | 2.0 |
| WV-40386 | 73.9 |
| WV-44359 | 3.62 uM |
| WV-44360 | 648.7 |
| WV-44361 | N.D. |

N.D. = not determined

In vitro, in GABA neurons. 4 day treatment. Table 11 shows %IC50 of knocking down MALAT1 mRNA in iCell Neurons, dose response [10 uM-25.6 pM, 5-fold dilution].

TABLE 12
Reduction of MALAT1 transcripts.

| ID | IC50 [nM] |
|---|---|
| WV-8587 | 197.4 |
| WV-15562 | 3.4 |
| WV-40579 | 3.9 |
| WV-40831 | 145.4 |
| WV-40832 | 45.0 |
| WV-40807 | 229.3 |
| WV-40808 | 156.0 |
| WV-40835 | 16.6 |
| WV-30938 | 35.2 |
| WV-39402 | 23.8 |
| WV-39291 | 32.6 |

In vitro, in GABA neurons. 4 day treatment. Table 12 shows %IC50 of knocking down MALAT1 mRNA in iCell Neurons, dose response [10 uM-25.6 pM, 5-fold dilution].

TABLE 13
Reduction of MALAT1 transcripts.

| ID | IC50 [nM] |
|---|---|
| WV-8587 | 65.9 |
| WV-15562 | 12.0 |
| WV-43245 | N.D. |
| WV-43246 | 40.2 |
| WV-43247 | 326.1 |
| WV-43248 | 17.7 |
| WV-43249 | 10.3 |
| WV-43250 | 5.0 |
| WV-43251 | 106.0 |
| WV-43252 | N.D. |
| WV-43253 | 621.7 |
| WV-43254 | 2.45 uM |
| WV-43255 | 1.22 uM |

In vitro, in GABA neurons. 4 day treatment. Table 13 shows %IC50 of knocking down MALAT1 mRNA in iCell Neurons, dose response [10 uM-25.6 pM, 5-fold dilution].

TABLE 14
Reduction of MALAT1 transcripts.

| Oligo | IC50 [nM] |
|---|---|
| WV-8587 | 198.9 |
| WV-15562 | 19.7 |
| WV-24104 | 237.5 |
| WV-43249 | 14.0 |
| WV-43250 | 52.8 |
| WV-43248 | 25.4 |
| WV-24109 | 15.0 |

In vitro, in GABA neurons. 4 day treatment. Table 14 shows %IC50 of knocking down MALAT1 mRNA in iCell Neurons, dose response [10 uM-1.0 pM, 5-fold dilution].

TABLE 15
Reduction of MALAT1 transcripts.

| ID | IC50 [nM] |
|---|---|
| WV-15562 | 32.4 |
| WV-15563 | 2.0 |
| WV-15885 | 1.4 uM |
| WV-15887 | 2.0 uM |
| WV-24104 | 301.8 |
| WV-24105 | 5.7 uM |
| WV-41422 | 1.0 uM |
| WV-41430 | N.D. |
| WV-44357 | 18.9 |
| WV-44370 | 20.2 |
| WV-43249 | 30.3 |
| WV-43250 | 38.9 |

In vitro, in GABA neurons. 4 day treatment. Table 15 shows %IC50 of knocking down MALAT1 mRNA in iCell Neurons, dose response [10 uM-25.6 pM, 5-fold dilution].

TABLE 16
Reduction of MALAT1 transcripts.

| ID | IC50 [nM] |
|---|---|
| WV-8587 | 162.9 |
| WV-15562 | 21.6 |
| WV-44468 | 63.5 |
| WV-44469 | 152.8 |
| WV-44470 | 226.4 |
| WV-45083 | 34.0 |
| WV-45084 | 139.2 |
| WV-45085 | 161.7 |
| WV-45086 | 26.8 |
| WV-45087 | N.D. |
| WV-45088 | 2.15 uM |
| WV-38642 | 30.7 |
| WV-44962 | 31.5 |
| WV-44964 | 94.9 |
| WV-44966 | 85.7 |

N.D. = not determined.

In vitro, in GABA neurons. 4 day treatment. Table 16 shows %IC50 of knocking down MALAT1 mRNA in iCell Neurons, dose response [10 uM-1 pM, 5-fold dilution].

In some embodiments, reduction of Malatl was observed in iCell neurons, with the following EC50 (uM): WV-8556 (1.4), WV-8587 (0.18), WV-11533 (0.02), WV-13303 (0.04), WV-13304 (0.01), WV-15562 (0.03), and WV-15563 (0.01). As demonstrated, oligonucleotides comprising non-negatively charged internucleotidic linkages as described herein, e.g., n001, can provide improved activity. In some embodiments, it was confirmed that cleavage was directed by RpSpSp in RNasH assay. In some embodiments, WV-8587, WV-11533, WV-13303, and WV-13304 provided two major cleavage sites within a region of a RNA oligonucleotide complementary to DNA portions of these oligonucleotides, while WV-8556 led to more major cutting sites within a region of the RNA oligonucleotide complementary to the DNA portion of WV-8556. In some embodiments, it was observed that non-negatively charged internucleotidic linkages, e.g., PN linkages such as n001, may increase potency. In some embodiments, the following EC50 (uM) were observed in iCell neurons: WV-8556 (1.4) , WV-8587 (0.18), WV-11533 (0.02), WV-15562 (0.03), WV-15563 (0.01), WV-30915 (0.07), WV-30916 (0.05), WV-38634 (0.04), WV-38635 (0.07), WV-38636 (0.03), WV-38637 (0.03), and WV-38638 (0.04). In some embodiments, the following EC50 (uM) was observed in iCell neurons: WV-8587 (0.36), WV-15562 (0.04), and WV-24104 (0.46). In some embodiments, the following EC50 (uM) was observed in iCell neurons: WV-15562 (0.01), WV-43249 (0.01), WV-43250 (0.001), and WV-43248 (0.02).

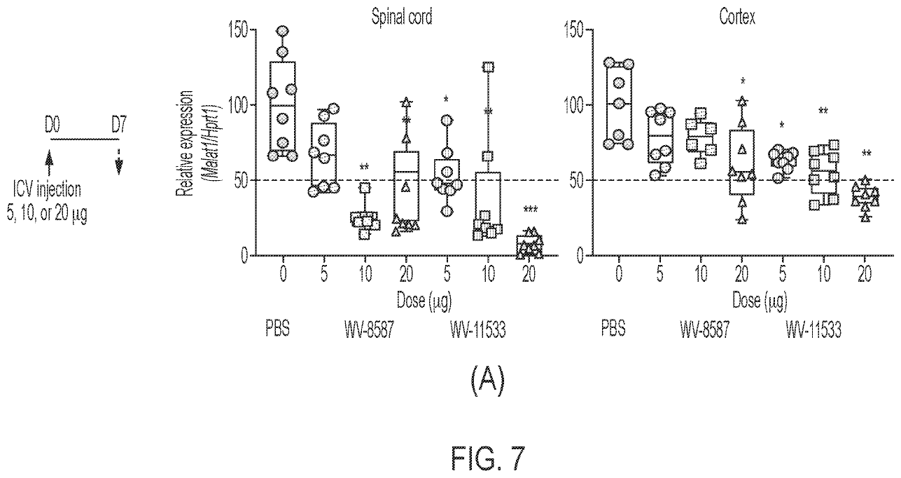

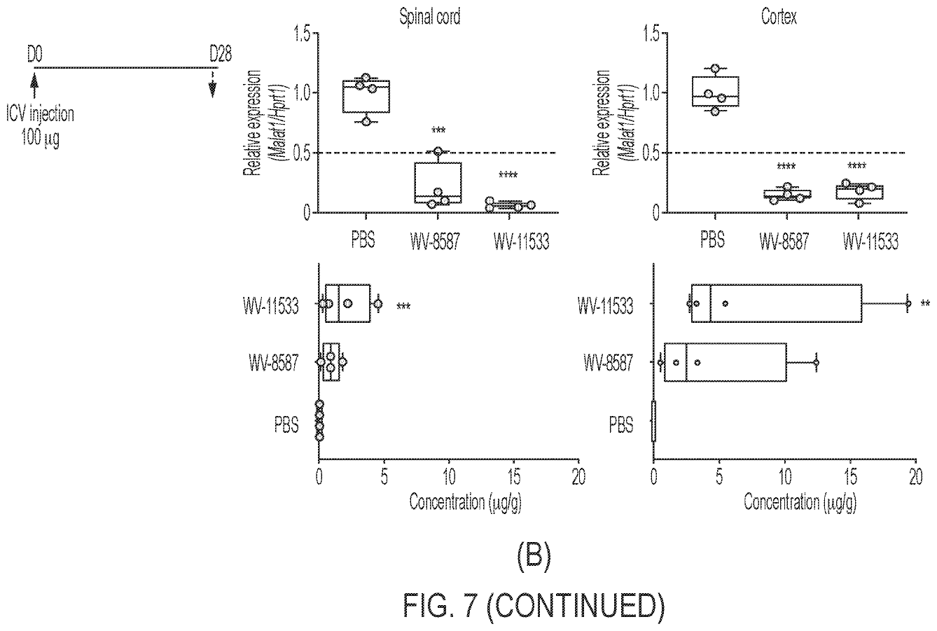

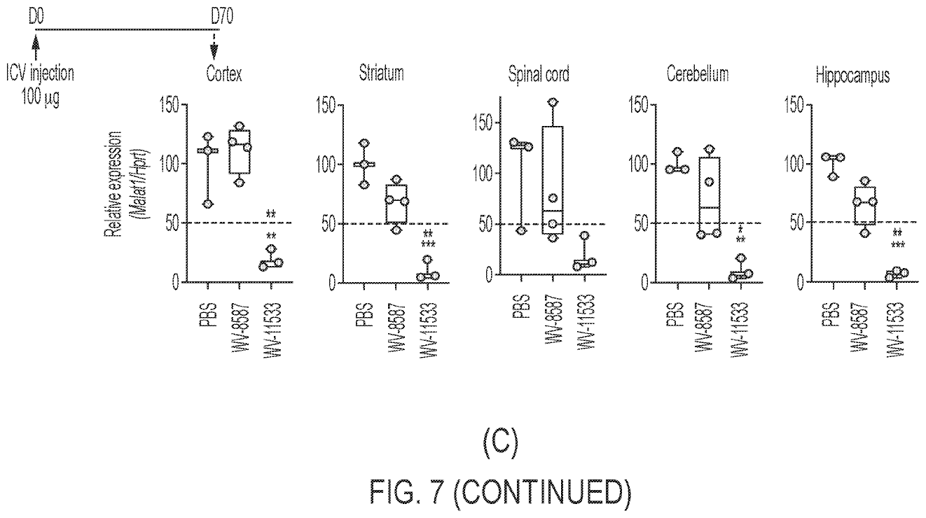

In some embodiments, Malatl expression was evaluated in wild-type mice administered with WV-8587 or WV-11533 in a single-dose, dose-escalation experiment. In some embodiments, activity was assessed in spinal cord and cortex 1-week after dosing. In some embodiments, in spinal cord a single 10 or 20 ug dose of WV-8587 decreased expression of Malatl by 50% or more compared with vehicle treatment. In some embodiments, a single 5 ug dose of WV-11533 was sufficient to decrease expression to a 50% threshold (FIG. 7, (A)), and a 20 ug dose decreased mean Malatl expression to just 8% of vehicle-treated controls. In cortex, a 20 ug dose of WV-8587 decreased expression to approximately 50% threshold (PBS: 100% mean Malatl expression; 20 ug : 61% mean expression), whereas a 5 ug dose of WV-11533 decreased Malatl expression comparably (PBS: 100% mean Malatl expression; 5 ug 63% mean expression). In some embodiments, incorporation of one or more non-negatively charged internucleotidic linkages such as n001 improved potency. To assess distribution in CNS, oligonucleotide concentrations and Malatl expression levels were assessed in spinal cord and cortex 4-weeks after a single 100 ug ICV injection. It was observed both WV-8587 and WV-11533 depleted Malatl RNA expression in spinal cord and cortex by —80% (spinal cord PBS mean 1.0-fold expression, WV-8587 mean 0.21-fold, WV-11533 mean 0.21-fold; cortex PBS mean 1.0-fold expression, WV-8587 mean 0.15-fold, WV-11533 mean 0.18-fold) (FIG. 7, (B)). Oligonucleotide concentrations for spinal cord and cortex from the same single dose experiment was assessed. In some embodiments, average concentration of WV-11533 was —2-fold higher than WV-8587 in both tissues (spinal cord: 2.0 ug/g and 0.9 ug/g, respectively; cortex: 7.7 ug/g and 4.5 ug/g, respectively) (FIG. 7, (B)). In some embodiments, it was observed that WV-11533 concentrations were significantly greater than vehicle-treated negative controls in both tissues (spinal cord P=0.0009, cortex P=0.0078, One-way ANOVA), whereas WV-8587 was not (spinal cord P=0.93, cortex P=0.12, One-way ANOVA). In some embodiments, WV-11533 was more durable. Malatl expression in CNS from wild-type mice 10 weeks after receiving a single 100 ug ICV dose. In some embodiments, differences in the activities of WV-8587 and WV-11533, in some cases, throughout the CNS was observed. In some embodiments, by 10-weeks post-injection in WV-8587-treated animals, Malatl expression had recovered so it was above a 50% threshold in all tissues (spinal cord mean 83% expression, cortex 112%, hippocampus 65%, cerebellum 70%, striatum 68%) and was statistically significantly different from vehicle-treated controls only in the hippocampus (FIG. 7, (C); spinal cord P=0.89, cortex P=0.75, hippocampus P=0.02, cerebellum P=0.29, striatum P=0.07). By contrast in WV-11533-treated animals, Malatl expression remained very low, below 20% of normal expression levels in all tissues evaluated and below 10% in striatum, cerebellum and hippocampus, at 10 weeks (spinal cord mean 20% expression, P=0.17; cortex 19%, P=0.005; hippocampus 6.8%, P=0.0001; cerebellum 10%, P=0.006; striatum 10%, P=0.0005, One-way ANOVA). In some embodiments, Malatl knockdown observed in cortex and spinal cord with WV-11533 at 4 weeks (e.g., about 80%) was unchanged at 10 weeks. Among other things, provided technologies, e.g., oligonucleotide comprising various non-negatively charged internucleotidic linkages such as n001, can be more potent, achieve higher tissue exposure and/or be more durable.

Various oligonucleotides and compositions were also assessed for modulating splicing. In one procedure, H2K cells were differentiated for 4 days, dosed for 3 hours, and then replaced with new media. The cells were further differentiated for 4 days prior to RNA Trizol extraction, cDNA preparation and Taqman multiplex analysis. Skipping values were interpolated from an absolute curve generated using gBlocks. It was observed that certain oligonucleotides and compositions, such as WV-28767, WV-28768, WV-28800 and WV-28801, can provide similar skipping levels to WV-11345, and about 2-fold of those observed for WV-10258. Additional data were provided in the Figures, demonstrating provided technologies can provide effective exon skipping as desired.

Table 17 shows % mouse DMD Ex23 mRNA skipping (at 3, 1, 0.3 and 0.1 uM oligonucleotide treatment) relative to total DMD Ex23 control.

TABLE 17
Certain exon skipping data.

| | 3 uM | | 1 uM | | 0.3 uM | | 0.1 uM | |
|---|---|---|---|---|---|---|---|---|
| ID | Rep1 | Rep2 | Rep1 | Rep2 | Rep1 | Rep2 | Rep1 | Rep2 |
| WV-10258 | 11.6 | 8.6 | 4.9 | 4.1 | 0.7 | 2 | 0.2 | 0.4 |
| WV-11345 | 16.5 | 18.1 | 10.4 | 9 | 3 | 3.3 | 0.8 | 0.9 |
| WV-28471 | N.D. | 17.4 | N.D. | 8.3 | N.D. | 3 | N.D. | 0.8 |
| WV-21218 | 24.7 | 24.3 | 16 | 17.6 | 5.4 | 4.6 | 2 | 1.3 |
| WV-28294 | N.D. | 22.5 | N.D. | 18.6 | N.D. | 4.4 | N.D. | 1.4 |
| WV-28472 | 0.1 | 4 | 0 | 0.7 | 0 | 2 | 0 | 0.1 |
| WV-28475 | 0.1 | 0 | 0 | 0 | 0 | 0 | 0 | 0.3 |
| WV-28476 | 2.2 | 2.5 | 1.1 | 0.2 | 0 | 0 | 0 | 0 |
| WV-28477 | 1.6 | 1.6 | 0.4 | 0.4 | 0.1 | 0 | 0 | 0 |

N = 2.
N.D.: Not determined.,
Rep: replicate

Table 18 shows % mouse DMD Ex23 mRNA skipping (at 3, 1, and 0.3 uM oligonucleotide treatment) relative to total DMD Ex23 control.

TABLE 18
Certain exon skipping data.

| | 3 uM | | 1 uM | | 0.3 uM | |
|---|---|---|---|---|---|---|
| ID | Rep1 | Rep2 | Rep1 | Rep2 | Rep1 | Rep2 |
| WV-22749 | 4.5 | 5.8 | 2.7 | 3.2 | 1.1 | 0.8 |
| WV-21218 | 19.2 | 20.6 | 11 | 13.3 | 4.1 | 6.8 |
| WV-43239 | N.D. | 11 | 5.9 | 6.8 | 1.6 | 3.1 |
| WV-43240 | 15.1 | 17.5 | 7.1 | 6.5 | 2.2 | 3.2 |
| WV-43241 | 4.4 | 6 | 1.7 | 3.9 | 0.1 | 1.9 |
| WV-43242 | 3.2 | 3.1 | 1 | 0.8 | 0.5 | 0.5 |
| WV-43243 | 1.1 | 1.9 | 0.8 | 0.4 | 0.2 | 0.1 |
| WV-43244 | 0 | 0.1 | 0.2 | 0.3 | 0.3 | 0 |
| WV-43287 | 13.9 | 13.3 | 9.9 | 6.1 | 4.5 | 2.2 |

N = 2.
N.D.: Not determined.,
Rep: replicate

### Example 29. Provided Technologies Can Provide High Activities

Among other things, provided technologies can provide high activities and/or various desired properties. Many technologies can be utilized to assess provided technologies in accordance with the present disclosure, e.g., in vitro assays, in vivo assays, biochemical assays, cell-based assays, animal models, clinical trials, etc. Certain useful technologies for assessing oligonucleotide activities and/or properties, and certain data confirming oligonucleotide activities and/or properties, are provided below as examples.

Example protocol for in vitro determination of oligonucleotide activity: For determination of oligonucleotide activity, oligonucleotides at specific concentration were gymnotically delivered to human primary hepatocytes plated at 96-well plates, with 10,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human MALAT1 transcripts, the following qPCR assay were utilized: ThermoFisher Taqman qPCR assay ID Hs00273907_sl. Human SFRS9 was used as normalizer (Forward 5′ TGGAATATGCCCTGCGTAAA 3′, Reverse 5′ TGGTGCTTCTCTCAGGATAAAC 3′, Probe 5′ TGGATGACACCAAATTCCGCTCTCA/3′. mRNA knockdown levels were calculated as %mRNA remaining relative to mock treatment. IC50 (nM): WV-8587: 3.5; WV-39603: 5.0; WV-39604: 7.4; WV-39605: 8.9; WV-12503: 0.17; and WV-39601: 0.17. In another assessment, IC50 (nM): WV-8587: 3.2; WV-44468: 6.4; WV-12503: 0.57; WV-45140: 0.27; WV-44470: 14.9. In some embodiments, two or more (e.g., three) additional moieties (e.g., carbohydrate moieties, ligands, etc.) in oligonucleotide (e.g., WV-12503, WV-39601, etc.) may provide improved delivery and/or efficacy compared to no or fewer carbohydrate moieties.

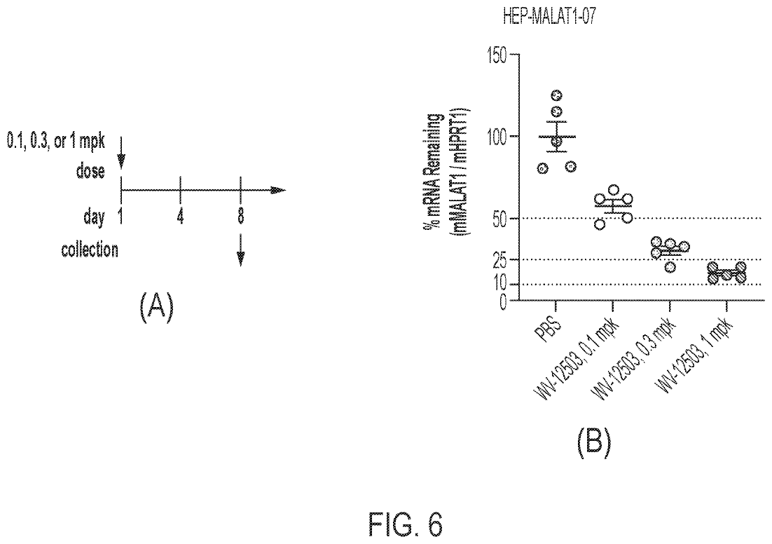

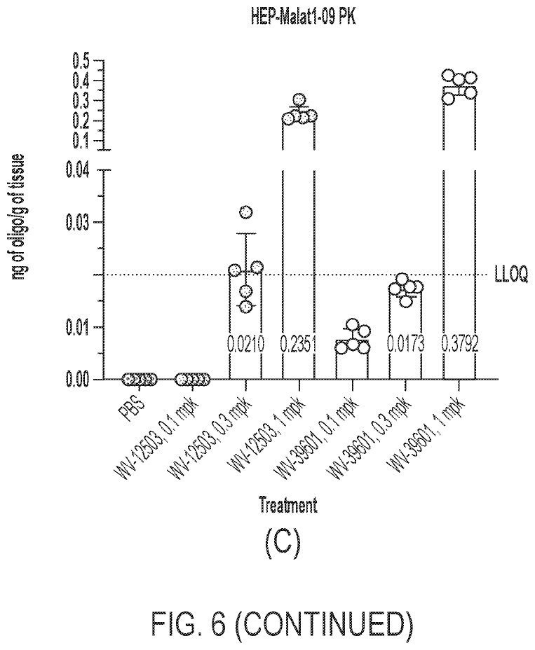

In vivo determination of mouse MALAT1 oligonucleotide activity: All animal procedures were performed under IACUC guidelines. To evaluate the potency and liver exposure of provided oligonucleotides and compositions, male 8-10 weeks of age C57BL/6 mice were dose at 0.1, 0.3 or 1 mg/kg at desired oligonucleotide concentration on Day 1 by subcutaneous administration. Animals were euthanized on Day 8 by CO₂ asphyxiation followed by thoracotomy. After cardiac perfusion with PBS, liver samples were harvested and flash-frozen in dry ice. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse MALAT1 mRNA, the following qPCR assay were utilized: ThermoFisher Taqman qPCR assay ID Mm01227912_sl. Mouse HPRT was used as normalizer (Forward 5′ CAAACTTTGCTTTCCCTGGTT 3′, Reverse 5′ TGGCCTGTATCCAACACTTC 3′, Probe 5′ACCAGCAAGCTTGCAACCTTAACC/3′.0ligonucleotide accumulation in liver was determined by hybrid ELISA. In vivo delivery and activities were confirmed by, e.g., data shown in FIG. 6. Dosing were performed as below:

| | | | | | | Total # | |
|---|---|---|---|---|---|---|---|
| | | | | Dosing | Dose | mice per | Necropsy |
| Group | Test Article | Dose | | Regimen | Volume | group | Timepoint |
| 1 | PBS | n/a | | s.c. (days 1) | 10 ml/kg | 5 | day 8 |
| 2 | WV-12503 | 0.1 | mpk | s.c. (days 1) | 10 ml/kg | 5 | day 8 |
| 3 | WV-12503 | 0.3 | mpk | s.c. (days 1) | 10 ml/kg | 5 | day 8 |
| 4 | WV-12503 | 1 | mpk | s.c. (days 1) | 10 ml/kg | 5 | day 8 |
| 5 | WV-39601 | 0.1 | mpk | s.c. (days 1) | 10 ml/kg | 5 | day 8 |
| 6 | WV-39601 | 0.3 | mpk | s.c. (days 1) | 10 ml/kg | 5 | day 8 |
| 7 | WV-39601 | 1 | mpk | s.c. (days 1) | 10 ml/kg | 5 | day 8 |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. An oligonucleotide having the structure of: 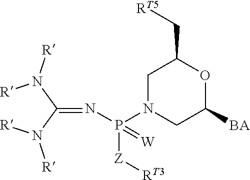
or a salt thereof, wherein:
BA is an optionally substituted or protected nucleobase;
R^{T5} is optionally substituted or protected hydroxyl, an optionally substituted or protected nucleotide moiety, an oligonucleotide moiety, R′, or an additional chemical moiety optionally connected through a linker;
R^{T3} is hydrogen, an optionally substituted or protected or nucleoside nucleotide moiety, an oligonucleotide moiety, R′, or an additional chemical moiety optionally connected through a linker;
W is O, S or Se;
Z is —O—, —S—, —N(R′)—;
each R^{L} is independently -L^{L}-R′ or —N═C(-L^{L}-R′)₂;
Ring A^{s} is an optionally substituted 3-30 membered, monocyclic, bicyclic or polycyclic ring having, in addition to the nitrogen, 0-10 heteroatoms;
each of L^{s}, L^{L1}, L^{L2} and L^{L} is independently L;
-Cy^{IL}- is -Cy-;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)O—, —OP(SR′)—, —OP(NR′)—, —OP(R′)—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

2. The oligonucleotide of claim 1, wherein the oligonucleotide has the structure of 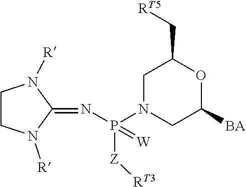
or a salt thereof.

3. An oligonucleotide, wherein the oligonucleotide has the structure of: 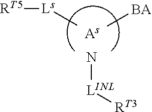
or a salt thereof, wherein:
BA is an optionally substituted or protected nucleobase;
R^{T5} is optionally substituted or protected hydroxyl, an optionally substituted or protected nucleotide moiety, an oligonucleotide moiety, R′, or an additional chemical moiety optionally connected through a linker;
R^{T3} is hydrogen, an optionally substituted or protected or nucleoside nucleotide moiety, an oligonucleotide moiety, R′, or an additional chemical moiety optionally connected through a linker;
L^{INL} is —Y—P^{L}(—X—R^{L})—Z—, —C(O)—O— wherein —C(O)— in bonded to a nitrogen atom,
—C(O)—N(R′)—, or -^{L1}-Cy^{IL}-L^{L2}-,
P^{L} is P, P(═W), P->B(-L^{L}-R^{L})₃, or P^{N};
W is O, N(-L^{L}-R^{L}), S or Se;
P^{N} is P═N—C(-L^{L}-R′)(=L^{N}-R′) or P═N-L^{L}-R^{L};
L^{N} is ═N-L^{L1}-, ═CH-L^{L1}- wherein CH is optionally substituted, or ═N⁺(R′)(Q⁻)-L^{L1}-;
Q⁻ is an anion;
each of X, Y and Z is independently —O—, —S—, —N(-L^{L}-R^{L})-, or L^{L};
each R^{L} is independently -L^{L}-R′ or —N═C(-L^{L}-R′)₂;
Ring A^{s} is an optionally substituted 3-30 membered, monocyclic, bicyclic or polycyclic ring having, in addition to the nitrogen, 0-10 heteroatoms;
each of L^{s}, L^{L1}, L^{L2} and L^{L} is independently L;
-Cy^{IL}- is -Cy-;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)o —, —OP(SR′)—, —OP(NR′)—, —OP(R′)—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in
addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

4. The oligonucleotide of any one of claims 1-3, wherein R^{T5} is optionally protected hydroxyl, an optionally substituted or protected nucleotide moiety or an oligonucleotide moiety, and R^{T3} is a nucleoside, nucleotide or oligonucleotide moiety connected to a solid support optionally through a linker.

5. An oligonucleotide, comprising:
one or more sugar units independently selected from:
a sugar having the structure of: 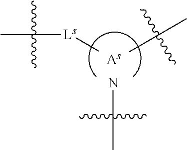
and an acyclic sugar, or
one or more modified internucleotidic linkages each independently having the structure of:
—Y—P^{L}(—X—R^{L})—Z—,
—C(O)—O— wherein —C(O)— in bonded to a nitrogen atom,
—C(O)—N(R′)—,or
-L^{L1}-Cy^{IL}-L^{L2}-,
wherein:
P^{L} is P, P(═W), P->B(-L^{L1}-R^{L})₃, or P^{N};
W is O, N(-L^{L}-R^{L}), S or Se;
P^{N} is P═N—C(-L^{L}-R′)(=L^{N}-R′) or P═N-L^{L}-R^{L};
L^{N} is ═N-L^{L1}-, ═CH-L^{L1}- wherein CH is optionally substituted, or ═N⁺(R′)(Q⁻)-L^{L1}-,
Q⁻ is an anion;
each of X, Y and Z is independently —O—, —S—, —N(-L^{L}-R^{L})—, or L^{L};
each R^{L} is independently -L^{L}-R′ or N═C(-L^{L}-R′)₂;
Ring A^{s} is an optionally substituted 3-30 membered, monocyclic, bicyclic or polycyclic ring having, in addition to the nitrogen, 0-10 heteroatoms;
each of L^{s}, L^{L1}L^{L2} and L;
-Cy^{IL}- is -Cy-;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁₄-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′))O——OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁-₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

6. The oligonucleotide of any one of the preceding claims, comprising a nucleoside unit comprising a morpholine unit, wherein the nitrogen of the morpholine unit is bonded to an internucleotidic linkage having the structure of —P(═W)(—N═C[N(R′)₂]₂)—O—.

7. The oligonucleotide of any one of the preceding claims, wherein an occurrence of P^{L} is P(═O) or P(═S).

8. The oligonucleotide of any one of the preceding claims, wherein an occurrence of P^{L} is P^{N}, P═N—C(-L^{L}-R′)(=L^{N}-R′), or P═N-L^{L}-R^{L}.

9. The oligonucleotide of any one of the preceding claims, wherein an occurrence of Y is a covalent bond, or wherein an occurrence of Y is —O—.

10. The oligonucleotide of any one of the preceding claims, wherein the oligonucleotide comprises a nucleoside unit comprising a morpholine unit, wherein the nucleoside unit has the structure of 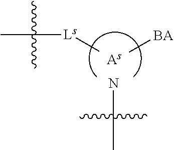
or a salt form thereof, wherein BA is a nucleobase, the N is bond to —C(O)—O—, wherein the —C(O)— is bonded to N.

11. The oligonucleotide of any one of the preceding claims, wherein each Z is —O—, and wherein an occurrence of W is O or S, or wherein each W is O.

12. The oligonucleotide of any one of the preceding claims, wherein an occurrence of -L^{L}-R^{L} is —N(R′)₂ or wherein R^{L} is —N═C(-L^{L}-R′)₂ or —N═C[N(R′)₂]₂.

13. The oligonucleotide of claim 12, wherein two R′ on the same nitrogen are taken together with their intervening atoms to form an optionally substituted, 3-30 membered, monocyclic, bicyclic or polycyclic ring having, in addition to the intervening atoms, 0-10 heteroatoms, or wherein R^{L} is

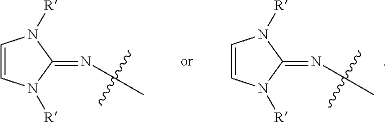

14. The oligonucleotide of any one of the preceding claims, wherein the oligonucleotide comprises 15-50 nucleobases.

15. The oligonucleotide of any one of the preceding claims, wherein the oligonucleotide comprises one or more (e.g., 1, 2, 3, 4, 5, 6, 7, 8, 9, 10, 11, 12, 13, 14, 15, 16, 17, 18, 19, 20) phosphorothioate internucleotidic linkages, and/or wherein at least 60%, 70%, 75%, 80%, 85%, 90% or 95% of all chiral internucleotidic linkages independently chirally controlled, and/or wherein at least 60%, 70%, 75%, 80%, 85%, 90% or 5% of all chirally controlled phosphorothioate internucleotidic linkages are Sp, and/or at least 60%, 70%, 75%, 80%, 85%, 90% or 95% of all chirally controlled non-negatively charged internucleotidic linkages are Rp, and/or wherein at least 60%, 70%, 75%, 80%, 85%, 90% or 95% of all chirally controlled internucleotidic linkages are Sp, and/or wherein at least 60%, 70%, 75%, 80%, 85%, 90% or 95% of all modified internucleotidic linkages are phosphorothioate internucleotidic linkages, and/or at least 60%, 70%, 75%, 80%, 85%, 90% or 95% of all modified internucleotidic linkages are phosphorothioate internucleotidic linkages having a Sp configuration, and/or wherein at least 60%, 70%, 75%, 80%, 85%, 90% or 95% of all internucleotidic linkages are phosphorothioate internucleotidic linkages, and/or wherein at least 60%, 70%, 75%, 80%, 85%, 90% or 95% of all internucleotidic linkages are phosphorothioate internucleotidic linkages having a Sp configuration.

16. The oligonucleotide of any one of the preceding claims, wherein the pattern of backbone chiral centers comprises [(Rp/Op)n(Sp)m]y, wherein each of n, m, and y is independently 1-50, and each Np is independently Rp or Sp, and/or wherein the pattern of backbone chiral centers comprises (Np)t[(Rp/Op)n(Sp)m]y, wherein each oft, n, m, and y is independently 1-50, and each Np is independently Rp or Sp, and/or wherein the pattern of backbone chiral centers comprises (Sp)t[(Rp/Op)n(Sp)m]y, wherein each oft, n, m, and y is independently 1-50, and/or wherein each Op indicates a linkage phosphorus being achiral in a natural phosphate linkage.

17. The oligonucleotide of any one of the preceding claims, comprising one or more (e.g., about or at least about 1, 2, 3, 4, 5, 6, 7, 8, 9, 10, 11, 12, 13, 14, 15, 16, 17, 18, 19, or 20; or about or at least about 5%, 10%, 15%, 20%, 25%, 30%, 35%, 40%, 45%, 50%, 55%, 60%, 65%, 70%, 75%, 80%, 85%, 90%, 95% of all, or all) sugars independently having the structure of 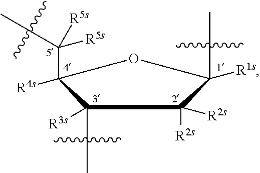
optionally:
wherein an occurrence of R^{5s} is —CH₃, or wherein in one an occurrence of sugar one R^{5s} is —CH₃, and the other is —H, and/or wherein the 5′-carbon is R or wherein the 5′-carbon is S; and/or wherein an occurrence of R^{5s} is —H; and/or each occurrence of R^{4s} is independently —H, or is taken together with a R^{2s} to form a bridge having the structure of -L^{b}-L^{b}-, wherein each L^{b} is independently L; and/or wherein each occurrence of R^{3s} is —H; and/or an occurrence of R^{2s} is —H, —F, —OR, wherein R is optionally substituted C₁₋₆ alkyl, —OMe, or —OCH₂CH₂OCH₃; and/or each occurrence of R^{1s} is —H.

18. The oligonucleotide of any one of the preceding claims, wherein the oligonucleotide consists of or comprises a structure of 5′-a first region-a second region-a third region-3′, wherein each of the regions independently comprises 1-20 (e.g., 1, 2, 3, 4, 5, 6, 7, 8, 9, 10, 11, 12, 13, 14, 15, 16, 17, 18, 19, 20) or more nucleosides.

19. The oligonucleotide of any one of the preceding claims, wherein the first region comprises 2, 3, 4, 5, 6, 7, 8, 9, 10, 11, 12, 13, 14, 15, 16, 17, 18, 19, or 20 or more nucleosides, and/or wherein the second region comprises 8 or more nucleosides, and/or the third region comprises 2, 3, 4, 5, 6, 7, 8, 9, 10, 11, 12, 13, 14, 15, 16, 17, 18, 19, or 20 or more nucleosides, and/or wherein the first region comprises one or more internucleotidic linkages each independently of the structure —O—P(O)[—N═C[N(R′)₂]₂]—O—, and/or wherein the first region comprises one or more n001 internucleotidic linkages, and/or wherein at least 10%, 20%, 30%, 40%, 50%, 60%, 70%, 75%, 80%, 85%, 90%, or 95%, or 100% of all sugars in a second region comprises 2′-OR modified sugars, wherein R is optionally substituted C₁₋₆ aliphatic, and/or each sugar in the second region is independently a natural DNA sugar, and/or wherein at least 10%, 20%, 30%, 40%, 50%, 60%, 70%, 75%, 80%, 85%, 90%, or 95%, or 100% of all sugars in a third region comprises 2′-OR modified sugars, wherein R is optionally substituted C₁₋₆ aliphatic.

20. The oligonucleotide of any one of the preceding claims, wherein the pattern of backbone chiral centers of the oligonucleotide or the second region is or comprises wherein the pattern is or comprises (Np)t[(Rp)n(Sp)m]y or (Sp)t[(Rp)n(Sp)m]y, wherein each oft, n and m is independently 1-50 and y is 1-10.

21. The oligonucleotide of any one of the preceding claims, wherein at least one n is 1 or wherein each n is 1, and/or wherein y is 1 or 2, and/or wherein t is 2 or more, and/or wherein each m is independently 2-20, and/or wherein each Rp in a pattern of backbone chiral centers is independently of a phosphorothioate internucleotidic linkage, and/or wherein each Sp in a pattern of backbone chiral centers is independently of a phosphorothioate internucleotidic linkage.

22. The oligonucleotide of any one of the preceding claims, wherein each nucleobase is independently optionally substituted A, T, C, G or U, or an optionally substituted tautomer of A, T, C, G or U.

23. The oligonucleotide of any one of the preceding claims, wherein the oligonucleotide chain is conjugated with an additional moiety which is or comprises a lipid moiety, a carbohydrate moiety, and/or a targeting moiety, and/or wherein the additional moiety is or comprises

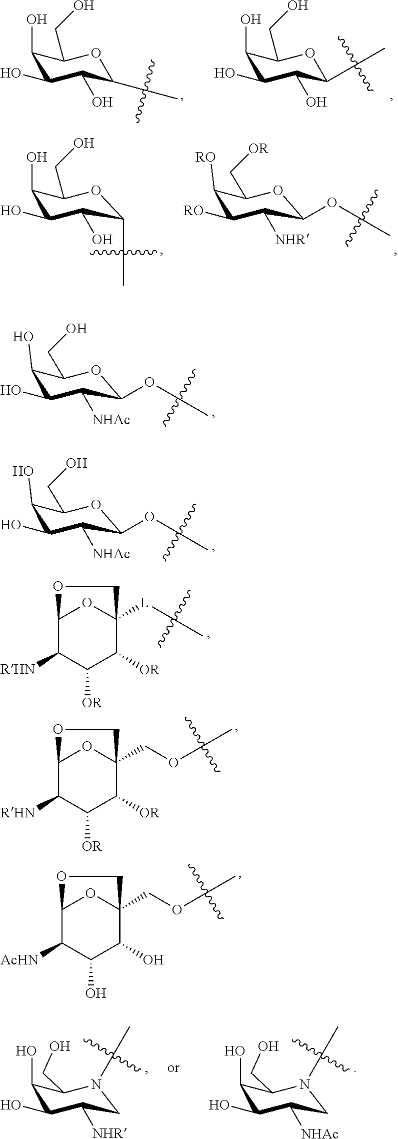

24. An oligonucleotide comprising 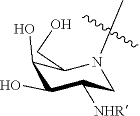
or a compound comprising 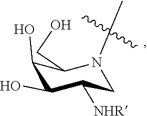
optionally wherein R′ is —Ac.

25. The oligonucleotide of any one of the preceding claims, wherein the oligonucleotide is in a form of a pharmaceutically acceptable salt.

26. The oligonucleotide of any one of the preceding claims, wherein each chirally controlled phosphorothioate internucleotidic linkage of the oligonucleotide independently has a diastereomeric purity of at least 85%, 90%, 95%, 96%, 97%, 98%, or 99%, and/or wherein the oligonucleotide has a diastereomeric purity of at least (DS)^{nc}, wherein DS is 55%-100% (e.g., about or at least about 55%, 60%, 70%, 80%, 85%, 90%, 95%, 96%, 97%, 98%, or 99%), and nc is the number of chirally controlled internucleotidic linkages, and/or wherein the oligonucleotide has a diastereomeric purity of about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%.

27. A chirally controlled oligonucleotide composition comprising a plurality of oligonucleotides, wherein the oligonucleotides share:
1) a common base sequence,
2) a common pattern of backbone linkages, and
3) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),
wherein about 1-100% of all oligonucleotides within the composition that share the common base sequence and common pattern of backbone linkages are the oligonucleotides of the plurality,
each oligonucleotide of the plurality is independently an oligonucleotide of any one of claims 1-25; or
a chirally controlled oligonucleotide composition comprising a plurality of oligonucleotides, wherein the oligonucleotides share:
1) a common constitution, and
2) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),
wherein the composition is enriched, relative to a substantially racemic preparation of oligonucleotides sharing the common constitution, for oligonucleotides of the plurality, and
each oligonucleotide of the plurality is independently an oligonucleotide of any one of claims 1-25; or
a chirally controlled oligonucleotide composition comprising a plurality of oligonucleotides, wherein the oligonucleotides share:
1) a common constitution, and
2) the same linkage phosphorus stereochemistry at one or more (e.g., 1, 2, 3, 4, 5, 6, 7, 8, 9, 10, 11, 12, 13, 14, 15, 16, 17, 18, 19, 20 or more) chiral internucleotidic linkages (chirally controlled internucleotidic linkages),
wherein about 1-100% of all oligonucleotides within the composition that share the common constitution are the oligonucleotides of the plurality, and
each oligonucleotide of the plurality is independently an oligonucleotide of any one of claims 1-25.
an oligonucleotide composition comprising a plurality of oligonucleotides, wherein:
each oligonucleotide of the plurality is independently a particular oligonucleotide or a salt thereof,
about 1-100% of all oligonucleotides within the composition that share the same constitution as the particular oligonucleotide or a salt thereof are oligonucleotides of the plurality, and
the particular oligonucleotide is an oligonucleotide of any one of claims 1-25; or
an oligonucleotide composition comprising a plurality of oligonucleotides, wherein:
each oligonucleotide of the plurality is independently a particular oligonucleotide or a salt thereof,
about 1-100% of all oligonucleotides within the composition that share the same base sequence as the particular oligonucleotide or a salt thereof are oligonucleotides of the plurality, and
the particular oligonucleotide is an oligonucleotide of any one of claims 1-25.

28. The composition of any one of the preceding claims, wherein the percentage is about or more than about (DS)^{nc}, wherein DS is 55%-100% (e.g., about or at least about 55%, 60%, 70%, 80%, 85%, 90%, 95%, 96%, 97%, 98%, or 99%), and nc is the number of chirally controlled internucleotidic linkages among oligonucleotides of the plurality; and/or wherein the percentage is 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-580%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%.

29. The composition of any one of the preceding claims, wherein oligonucleotides of the plurality are each independently in a pharmaceutically acceptable salt form.

30. A pharmaceutical composition comprising or delivering an oligonucleotide or a composition of any one of the preceding claims and a pharmaceutically acceptable carrier.

31. A method for modulating expression, level and/or activity of a target nucleic acid and/or a product thereof, comprising contacting the target nucleic acid with an oligonucleotide or composition of any one of the preceding claims, wherein the base sequence of the oligonucleotide, or the common base sequence of oligonucleotides of a plurality in a composition, is complementary to that of the target nucleic acid; or
a method, comprising administering to a system expressing a target nucleic acid an oligonucleotide or composition of any one of the preceding claims, wherein the base sequence of the oligonucleotide, or the common base sequence of oligonucleotides of a plurality in a composition, is complementary to that of the target nucleic acid.

32. The method of claim 31, wherein expression, level and/or activity of a target nucleic acid and/or a product thereof is reduced, and/or wherein a product is mRNA, and/or wherein a product is a protein; and/or wherein expression, level and/or activity of a product is increased, wherein the product is mRNA or a protein encoded thereby; and/or wherein the mRNA is a product of splicing modulation; and/or wherein the mRNA is a product of exon skipping; and/or wherein the system is a human.

33. A compound of formula AC-I, AC-I-a, AC-I-b, AC-I-c, AC-I-d, AC-I-e or a salt thereof, or a compound selected from

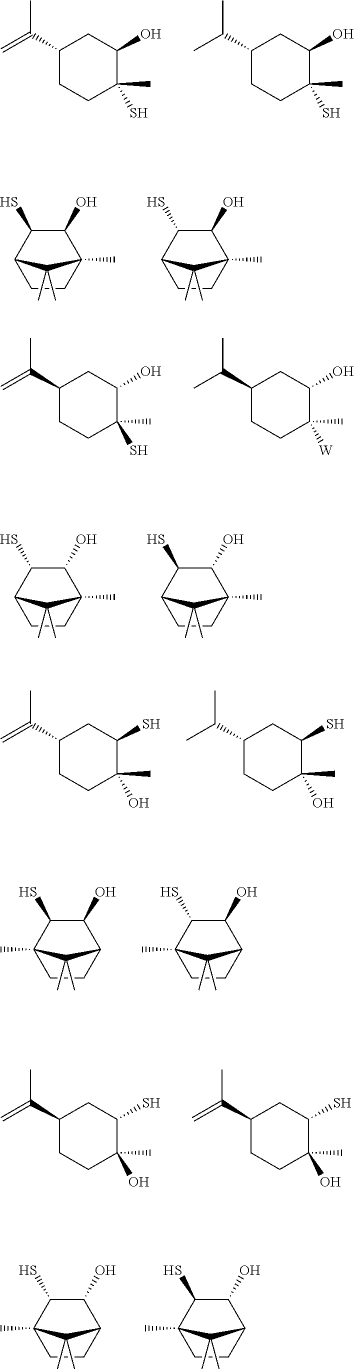

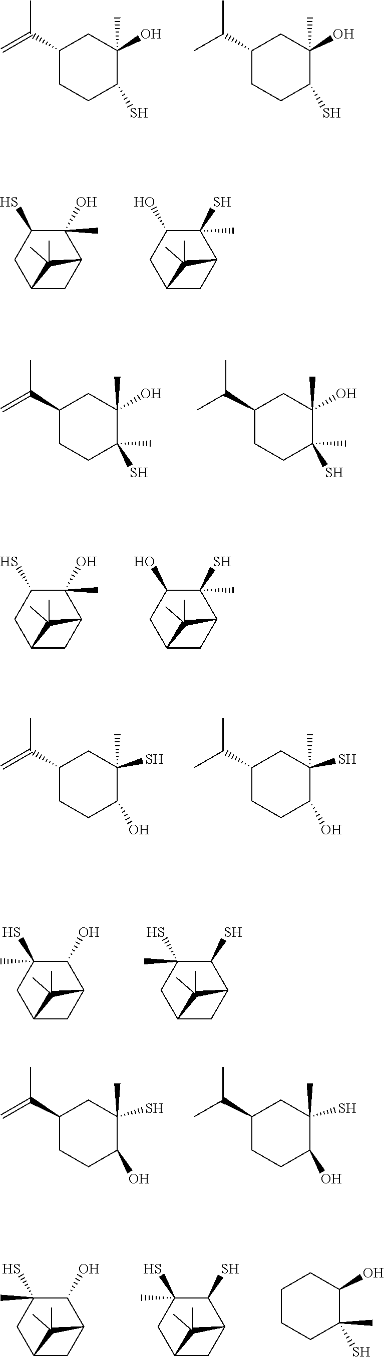

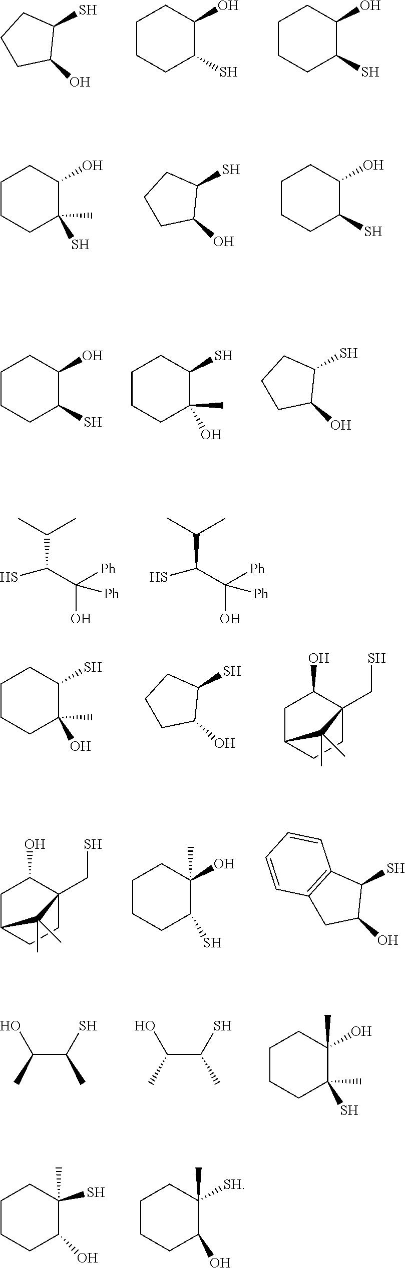

34. A compound having the structure of LG-I: 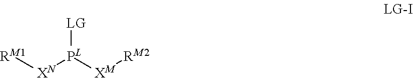
or a salt thereof, wherein:
LG is a leaving group;
each of X^{M} and X^{N} is independently -L-O—, -L-S— or -L-NR^{MN}—;
P^{L} is P, P(═W), P->B(-L^{L}-R^{L})₃, or P^{N};
W is O, N(-L^{L}-R^{L}), S or Se;
P^{N} is P═N—C(-L^{L}-R′)(=L^{N}-R′) or P═N-L^{L}-R^{L};
L^{N} is ═N-L^{L1}-,=CH-L^{L1}- wherein CH is optionally substituted, or ═N⁺(R′)(Q⁻)-L^{L1}-;
each L″ is independently L;
Q⁻ is an anion;
each of R^{M1}, and R^{M2} and R^{MN} is independently -L^{M}-R^{M}.
each R^{L} is independently -L^{L}-R′ or —N═C(-L^{L}-R′)₂,
each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
each of L^{L} and L^{M} is independently L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted, 3-30 membered, monocyclic, bicyclic or polycyclic ring having 0-10 heteroatoms;
each Cy^{L} is independently an optionally substituted, trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

35. The compound of claim 33, wherein X^{M} is —S— or —NO^{MN}—, and/or wherein R^{M1} and R^{M2} are taken together with their intervening atoms to form an optionally substituted, 3-30 membered, monocyclic, bicyclic or polycyclic ring having, in addition to the intervening atoms, 0-10 heteroatoms, and/or wherein R^{M1}, R^{M2} and R^{MN} are taken together with their intervening atoms to form an optionally substituted 3-30 membered, monocyclic, bicyclic or polycyclic ring having 0-10 heteroatoms, and/or wherein LG is —Cl, —N(R′)₂, or —N(iPr)₂.

36. A compound having the structure of LG-II: 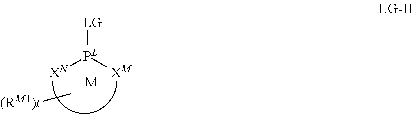
or a salt thereof, wherein:
LG is a leaving group;
each of X^{M} and X^{N} is independently -L-O—, -L-S— or -L-NR^{MN}—;
P^{L} is P, P(═W), P->B(-L^{L}-R^{L})₃, or P^{N};
W is O, N(-L^{L}-R^{L}), S or Se;
each R^{L} is indendently -L^{L}-R′ or —N═C(-L^{L}-R′)₂;
P^{N} is P═N—C(-L^{L}-R′)(=L^{N}-R′) or P═N-L^{L}-R^{L};
L^{N} is ═N-L^{L1}-, ═CH-L^{L1}- wherein CH is optionally substituted, or ═N⁺(R′)(Q⁻)-L^{L1}-;
each L^{L1} is independently L;
Q⁻ is an anion;
each of R^{M1}, R^{M2} and R^{MN} is independently -L^{M}-R^{M};
each R^{L} is independently -L^{L}-R′ or —N═C(-L^{L}-R′)₂;
each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
t is 0-10;
each of L^{L} and L^{M} is independently L;
Ring M is an optionally substituted 3-30 membered, monocyclic, bicyclic or polycyclic ring having 0-10 heteroatoms;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)O—, —OP(SR′)—, —OP(NR′)—, —OP(R′)—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

37. The compound of claim 36, wherein X^{M} is —S— or —NR^{MN}—, and/or Ring M is 5-membered or 6-membered, and/or Ring M is saturated, and/or wherein Ring M has no heteroatoms in addition to the intervening atoms, and/or wherein t is 2; and/or wherein each R^{M1} is independently R; and/or wherein each R^{M1} is independently optionally substituted C₁₋₃₀ aliphatic; and/or wherein 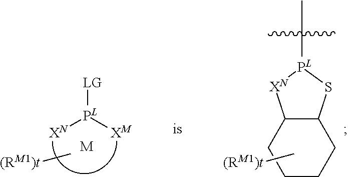
and/or wherein 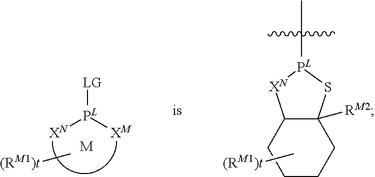
and/or wherein 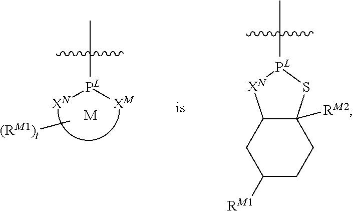
wherein R^{M1} and R^{M2}are trans; and/or wherein 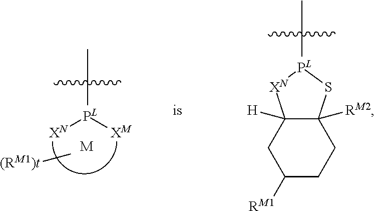
wherein the H and R^{M2} are trans; and/or wherein 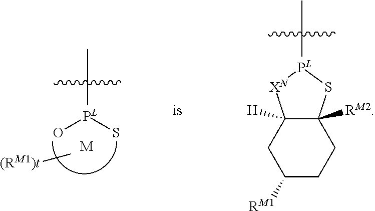
wherein X^{M} is —S—; and/or wherein R^{M1} is —C(CH₃)═CH₂ or —CH₃; and/or wherein 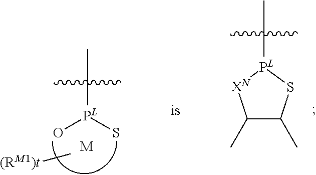
and/or wherein LG is —Cl, —N(R′)₂, or —N(iPr)₂.

38. A compound having the structure of formula M-I: 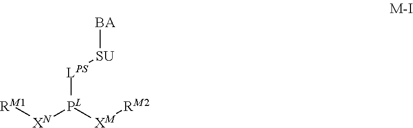
or a salt thereof, wherein:
each of X^{M} and X^{N} is independently -L-O—, -L-S— or -L-NR^{MN}—;
P^{L} is P, P(═W), P->B(-L^{L}-R^{L})₃, or P^{N};
W is O, N(-L^{L}-R^{L}), S or Se;
each R^{L} is indendently -L^{L}-R′ or —N═C(-L^{L}-R′)₂;
P^{N} is P═N—C(-L^{L}-R′)(=L^{N}-R′) or P═N-L^{L}-R^{L};
L^{N} is ═N-L^{L1}-, ═CH-L^{L1}- wherein CH is optionally substituted, or ═N⁺(R′)(Q⁻)-L^{L1}-;
each L^{L1} is independently L;
Q⁻ is an anion;
each of R^{M1}, R^{M2} and R^{MN} is independently -L^{M}-R^{M};
each R^{L} is independently -L^{L}-R′ or —N═C(-L^{L}-R′)₂;
each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
each of L^{L} and L^{M} is independently L;
BA is a nucleobase;
SU is a sugar;
L^{PS} is a L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)—, —OP(O)(SR′)—, —OP(O)(R′)—, —OP(O)(NR′)—, —OP(OR′)O—, —OP(SR′)—, —OP(NR′)—, —OP(R′)—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

39. The compound of claim 38, wherein X^{M} is —S— or —NR^{MN}—, and/or wherein R^{M1} and R^{M2} are taken together with their intervening atoms to form an optionally substituted, 3-30 membered, monocyclic, bicyclic or polycyclic ring having, in addition to the intervening atoms, 0-10 heteroatoms, and/or wherein the formed ring is 5-membered or 6-membered, and/or the formed ring is substituted, and/or wherein R^{M1}, R^{M2} and R^{MN} are taken together with their intervening atoms to form an optionally substituted 3-30 membered, monocyclic, bicyclic or polycyclic ring having 0-10 heteroatoms.

40. A compound having the structure of formula M-II: 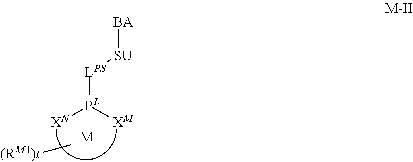
or a salt thereof, wherein:
each of X^{M} and X^{N} is independently -L-O—, -L-S— or -L-NR^{MN}—;
P^{L} is P, P(═W), P->B(-L^{L}-R^{L})₃, or P^{N};
W is O, N(-L^{L}-R^{L}), S or Se;
P^{N} is P═N—C(-L^{L}-R′)(=L^{N}-R′) or P═N-L^{L}-R^{L};
L^{N} is ═N-L^{L1}-, ═CH-L^{L1}- wherein CH is optionally substituted, or ═N⁺(R′)(Q⁻)-L^{L1}-;
each L^{L1} is independently L;
Q⁻ is an anion;
each of R^{M1} and R^{MN} is independently -L^{M}-R^{M};
each R^{L} is independently -L^{L}-R′ or —N═C(-L^{L}-R′)₂;
each R^{M} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
t is 0-10;
each of L^{L} and L^{L1} is independently L;
Ring M is an optionally substituted 3-30 membered, monocyclic, bicyclic or polycyclic ring having 0-10 heteroatoms;
BA is a nucleobase;
SU is a sugar;
L^{PS} is a L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —S(O)—, —S(O)₂—, —S(O)₂N(R′)—, —C(O)S—, —C(O)—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

41. The compound of claim 40, wherein X^{M} is —S— or —NR^{MN}—, and/or Ring M is 5-membered, and/or M is 6-membered, and/or wherein t is 1-10 or t is 2, and/or each R^{M1} is independently R or independently optionally substituted C₁₋₃₀ aliphatic.

42. The compound of claim 40, wherein 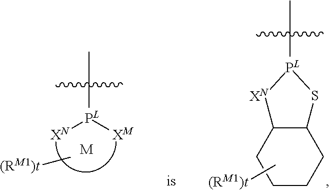
and/or wherein 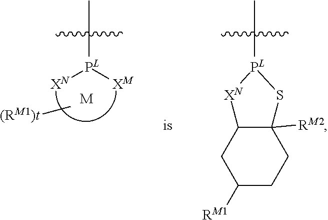
and/or wherein 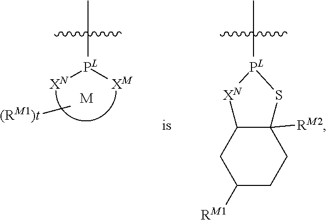
wherein R^{M1}
and R^{M2} are trans, and/or wherein 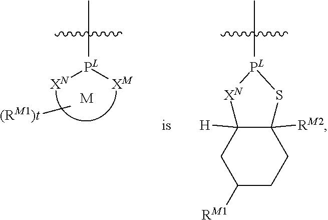
wherein the H and R^{M2} are trans, and/or wherein 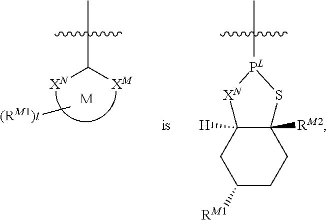
and/or wherein 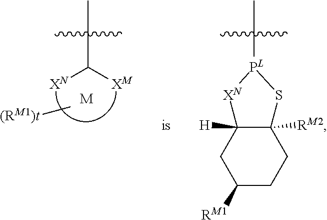
and/or wherein R^{M1} is —C(CH₃)═CH₂, and/or R^{M2} is —CH₃, and/or wherein

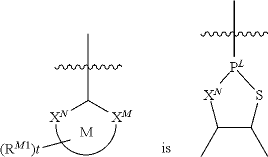

43. The compound of any one of claims 40-42, wherein SU is 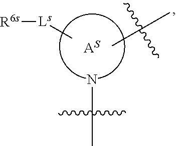
wherein:
R^{6s} is R^{s};
each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
Ring A^{s} is an optionally substituted 3-30 membered, monocyclic, bicyclic or polycyclic ring having, in addition to the nitrogen, 0-10 heteroatoms;
L^{s} is L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

44. The compound of claim 43, wherein the N is bonded to P^{L}, and/or wherein L^{s} is —C(R^{5s})₂, wherein each R^{5s} is independently R^{s}, and/or wherein L^{s} is optionally substituted —CH₂—, and/or wherein L^{s} is —CH₂—, and/or wherein 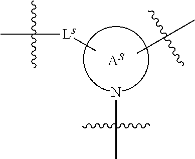
is optionally substituted 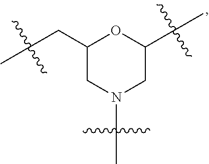 [SMILES: *CC1CN(*)CC(*)O1]
and/or wherein 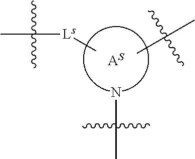
is optionally substituted 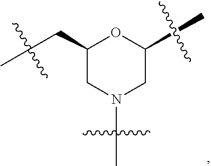
and/or wherein 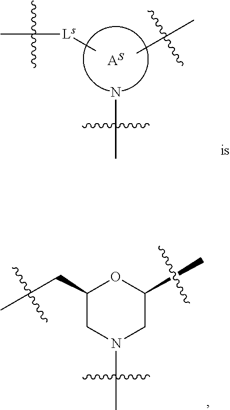
and/or wherein

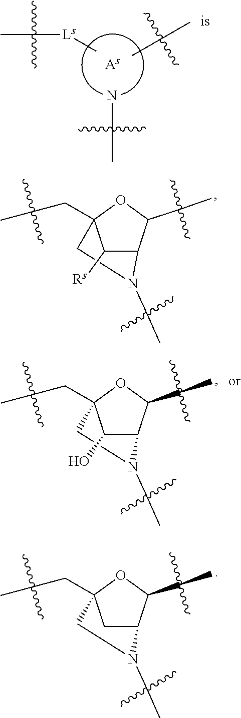

45. The compound of any one of claims 33-42, wherein SU is 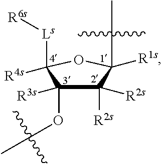
wherein:
each of R^{1s}, R^{2s}, R^{3s}, R^{4s}, R^{5s}, and R^{6s} is independently R^{s};
each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
L^{s} is L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

46. The compound of claim 45, wherein Cl is bonded to BA, and/or wherein L^{s} is —C(R^{5s})₂, wherein each R^{5s} is independently R^{s}, and/or wherein L^{s} is optionally substituted —CH₂—, and/or wherein L^{s} is —CH₂—, and/or wherein R¹s is —H, and/or wherein R^{3s} is —H, and/or wherein SU is 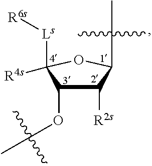
and/or wherein R^{2s} is —H, —OR, wherein R is C₁₋₆ aliphatic, —OMe, or -MOE, and/or R^{4s} is —H, and/or wherein R^{2s} and R^{4s} are taken together to form-L-, and/or wherein R^{2s} and R^{4s} are taken together to form-L-, wherein L is 2′-O—CH₂-4′, wherein the —CH₂— is optionally substituted.

47. The compound of claim 45, wherein SU is an acyclic sugar or -L^{PS}-SU′-R^{6s}, and/or wherein R^{6s} is —OH protected for oligonucleotide synthesis, and/or wherein R^{6s} is DMTrO—.

48. The compound of any one of the preceding claims, wherein X^{N} is —O—, —S—, or —NR^{MN}—.

49. The compound of any one of the preceding claims, wherein P^{L} is P, or P^{L} is P(═W), wherein W is O, or P^{L} is P(═W), wherein W is S, or wherein P^{L} is P(═W), wherein W is Se, or wherein P^{L} is P(═W), wherein W is N(-L^{L}-R^{L}), or wherein P^{L} is P^{N}, or wherein P^{L} is P═N—C(-L^{L}-R′)[═N⁺(R′)(Q⁻)-L^{L1}-R′], or wherein P^{L} is P═N—C(-L^{L}-R′)[═N⁺(R′)(Q⁻)-L^{L1}-R′)], or wherein P^{L} is P═N—C[N(R′)₂][═N⁺(R′)(Q⁹⁻)-L^{L1}-R′], or wherein P^{L} is P═N—C[N(R′)₂][═N⁺(R′)₂(Q⁻)], and/or wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms, and/or wherein one R′ on one N and one R′ or the other N are taken together with their intervening atoms to form an optionally substituted, 5-membered, monocyclic, bicyclic or polycyclic ring having, in addition to the intervening atoms, 0 heteroatoms, and/or wherein the formed ring is saturated, and/or wherein P^{N} is 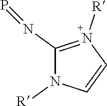
Q⁻, and/or wherein P^{N} is 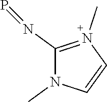
Q⁻, and/or wherein P^{N} is 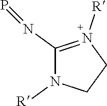
and/or wherein P^{N} is 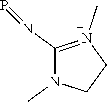
and/or wherein Q⁻ is PF₆⁻.

50. A compound having the structure of formula M-III:
BA-SU—C(O)-LG^{M}, M-III
or a salt thereof, wherein:
BA is a nucleobase;
SU is a sugar; and
LG^{M} is a leaving group.

51. The compound of claim 50, wherein LG^{M} is optionally substituted heteroaryl, and/or wherein LG^{M} is optionally substituted 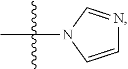 [SMILES: *n1ccnc1]
and/or wherein LG^{M} is 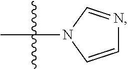 [SMILES: *n1ccnc1]
and/or wherein LG^{M} is optionally substituted 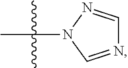 [SMILES: *n1cncn1]
and/or wherein LG^{M} is 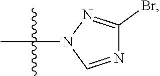 [SMILES: *n1cnc(Br)n1]
and/or wherein SU is 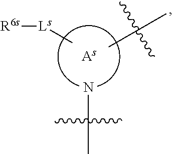
wherein:
R^{6s} is R^{s};
each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
Ring A^{s} is an optionally substituted 3-30 membered, monocyclic, bicyclic or polycyclic ring having, in addition to the nitrogen, 0-10 heteroatoms;
L^{s} is L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

52. The compound of claim 51, wherein the N is bonded to —C(O)—R^{M}, and/or L^{s} is —C(R^{5s})₂, wherein each R^{5s} is independently R^{s}, and/or wherein L^{s} is optionally substituted —CH₂—, and/or wherein L^{s} is —CH₂—, and/or wherein 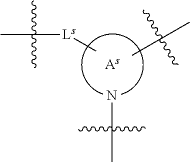
is optionally substituted 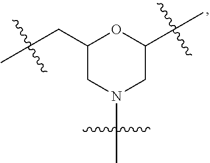 [SMILES: *CC1CN(*)CC(*)O1]
and/or wherein 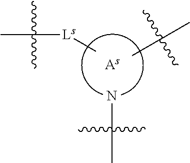
is optionally substituted 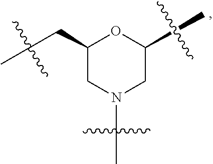
and/or wherein 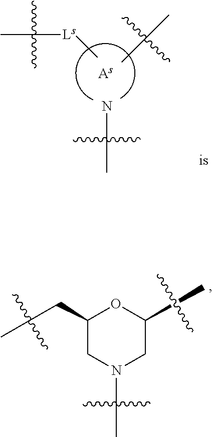
and/or wherein

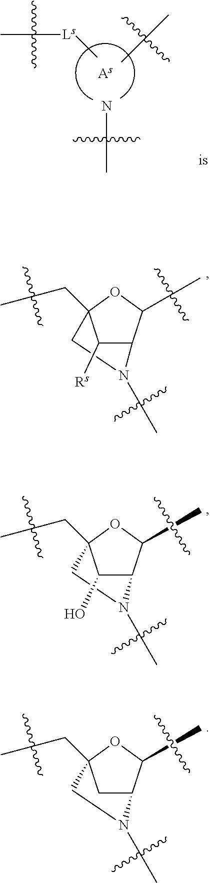

53. The compound of claim 51, wherein SU is 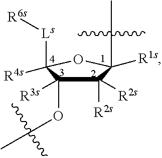
wherein:
each of R^{1s}, R^{2s}, R^{3s}, R^{4s}, R^{5s}, and R^{6s} is independently R^{s};
each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
L^{s} is L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

54. The compound of claim 53, wherein Cl is bonded to BA, and/or wherein L^{s} is —C(R^{5s})₂, wherein each R^{5s} is independently R^{s}, and/or wherein L^{s} is optionally substituted —CH₂—, and/or wherein L^{s} is —CH₂—, and/or wherein R^{1s} is —H, and/or wherein R^{3s} is —H, and/or wherein SU is 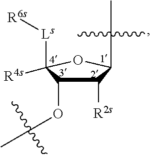
and/or wherein R^{2s} is —H, —OR, wherein R is C₁₋₆ aliphatic, —OMe, or -MOE, and/or R^{4s} is —H, and/or wherein R^{2s} and R^{4s} are taken together to form-L-, and/or wherein R²S and R^{4s} are taken together to form-L-, wherein L is 2′-O—CH₂-4′, wherein the —CH₂— is optionally substituted.

55. The compound of claim 51, wherein SU is an acyclic sugar or -L^{PS}—SU′- R^{6s}, and/or wherein R6S is —OH protected for oligonucleotide synthesis, and/or wherein L^{PS} is —O—, —NR′—, or a covalent bond, and/or wherein R^{6s} is —O-L-R′, and/or wherein R^{6s} is —OH protected for oligonucleotide synthesis, and/or wherein R^{6s} is DMTrO—.

56. A method for preparing a compound of any one of claims 34-37, comprising contacting a compound of claim 33 with a second compound.

57. The method of claim 56, wherein the second compound is PCl₃.

58. A method, comprising a coupling step that comprises:
contacting a first compound with a second compound comprising a hydroxyl group or an amino group, wherein the first compound is a compound any one of claims 49-55; or
a method, comprising a coupling step that comprises:
contacting a first compound with a second compound comprising a hydroxyl group or an amino group in the presence of a base, wherein the first compound is a compound of claim 49.

59. The method of claim 58, wherein the P of the P^{N} in the first compound forms a bond with the O of the —OH of the second compound.

60. A method, comprising a coupling step that comprises:
contacting a first composition with a second compound comprising a hydroxyl group or an amino group, wherein the first composition is prepared by a method comprising contacting a compound of claim 49 with a compound of formula AZ-1:
N₃—C(-L^{L}-R′)[═N⁺(R′)(Q⁻)-L^{L1}-R′]. AZ-I

61. The method of claim 58, wherein a first compound is prepared by contacting a compound of claim 49 with a compound having the structure of AZ-1:
N₃—C(-L^{L}-R′)[═N⁺(R′)(Q⁻)-L^{L1}-R′]. AZ-I

62. The method of claim 58, wherein a first compound is utilized without isolation and/or purification, or the method of any one of claims 60-62, wherein a compound of formula AZ-I is a compound of formula N₃—C(-L^{L}-R′)[═N⁺(R′)(Q⁻)—R′], or the method of any one of claims 60-62, wherein a compound of formula AZ-I is a compound of formula N₃—C[N(R′)₂][═N⁺(R′)(Q⁻)-L^{L1-}R′], or the method of any one of claims 60-62, wherein a compound of formula AZ-I is a compound of formula N₃—C[N(R′)₂][═N⁺(R′)₂(Q⁻)], or the method of any one of claims 60-62, wherein a compound of formula AZ-I is a compound of formula N₃—C(-L^{L}-R′)[═N⁺(R′)₂(Q⁻)].

63. The method of any one of claims 60-62, wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms, and/or wherein one R′ on one N and one R′ or the other N are taken together with their intervening atoms to form an optionally substituted, 5-membered, monocyclic, bicyclic or polycyclic ring having, in addition to the intervening atoms, 0 heteroatoms, and/or wherein the formed ring is saturated, and/or wherein a compound of formula AZ-1 is 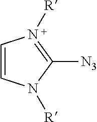
Q⁻, and/or wherein a compound of formula AZ-1 is 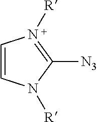
Q⁻, and/or wherein a compound of formula AZ-1 is 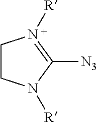
and/or wherein a compound of formula AZ-1 is 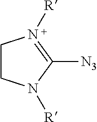
Q⁻, and/or wherein Q⁻ is PF₆⁻.

64. The method of any one of claims 58-63, wherein the contacting produces a third compound comprising —P(═W)(—X-L^{L}-R^{L})—Z—, and/or wherein Z is —O—, and/or wherein W is O, and/or wherein an occurrence of X is a covalent bond, and R^{L} is —N═C(-L^{L}-R′)₂, and/or wherein an occurrence of -L^{L}- is —N(R′)—, and/or wherein R^{L} is N═C[N(R′)₂]₂, and/or wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms, and/or wherein one R′ on one N and one R′ or the other N are taken together with their intervening atoms to form an optionally substituted, 5-membered, monocyclic, bicyclic or polycyclic ring having, in addition to the intervening atoms, 0 heteroatoms, and/or wherein the formed ring is saturated, and/or wherein R^{L} 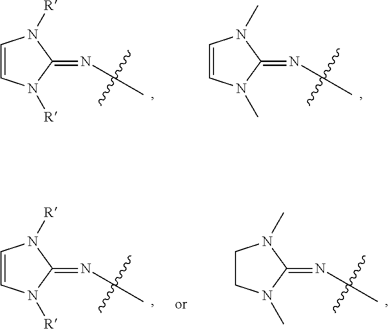
and/or wherein the P of —P(═W)(—X-L^{L}-R^{L})—Z— is the P of the P^{N} of the first compound.

65. The method of any one of claims 58-64, comprising converting P^{L} in a first compound or composition which P^{L} is P═N—C(-L^{L}-R′)[═N⁺(R′)(Q⁻)-L^{L1}-R′] into P—N═C(-L^{L}-R′)[N(R′)-L^{L1}-R′], or comprising converting P^{L} in a first compound or composition which P^{L} is P═N—C(-L^{L}-R′)[═N⁺(R′)(Q⁻)—R′] into P—N═C(-L^{L}-R′)[—N(R′)₂], or comprising converting P^{L} in a first compound or composition which P^{L} is P═N—C[N(R′)₂][═N⁺(R′)(Q⁻)-L^{L1}-R′] into P═N—C[N(R′)₂][—N(R′)-L^{L1}-R′], or comprising converting P^{L} in a first compound or composition which P^{L} is P═N—C[N(R′)₂][═N⁺(R′)₂(Q⁻)] into P═N—C[N(R′)₂][—N(R′)₂], optionally wherein one R′ on one N and one R′ or the other N are taken together with their intervening atoms to form an optionally substituted, 3-30 membered, monocyclic, bicyclic or polycyclic ring having, in addition to the intervening atoms, 0-10 heteroatoms, and/or wherein one R′ on one N and one R′ or the other N are taken together with their intervening atoms to form an optionally substituted, 5-membered, monocyclic, bicyclic or polycyclic ring having, in addition to the intervening atoms, 0 heteroatoms, and/or wherein the formed ring is saturated, and/or wherein R^{L} is 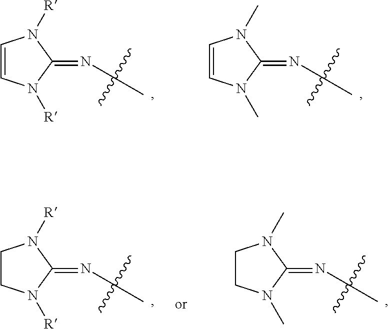
and/or wherein the P of —P(═W)(—X-L^{L}-R^{L})—Z— is the P of the P^{N} of the first compound, and/or wherein Q⁻ is PF₆⁻.

66. A method, comprising a coupling step that comprises:
contacting a first compound with a second compound comprising a hydroxyl group or amino group, wherein the first compound is a compound of claim 49; or
a method, comprising a coupling step that comprises:
contacting a first composition with a second compound comprising a hydroxyl group or an amino group, wherein the first composition is prepared by a method comprising contacting a compound of claim 49 with sulfurization agent.

67. The method of claim 66, wherein a first compound is prepared by contacting a compound of claim 49 with a sulfurization agent, and/or wherein a first compound is utilized without isolation and/or purification; and/or wherein a sulfurization agent is 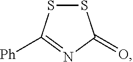 [SMILES: O=c1nc(-c2ccccc2)ss1]
and/or wherein the contacting produces a third compound comprising —P(═W)(—X-L^{L}-R^{L})—Z—, and/or wherein Z is —O—, and/or wherein W is O, and/or wherein —X-L^{L}-R^{L} is —S—H.

68. A method, comprising a coupling step that comprises:
contacting a first compound with a second compound comprising a hydroxyl or amino group, wherein the first compound is a compound of any one of claim 50-55.

69. The method of any one of claims 58-68, wherein the second compound is or comprises a nucleoside comprising a —OH group, and/or wherein the second compound is or comprises an oligonucleotide comprising a —OH group, and/or wherein the second compound is or comprises a nucleoside comprising an amino group, and/or wherein the second compound is or comprises an oligonucleotide comprising an amino group.

70. The method of any one of claims 60-69, wherein the nucleoside or oligonucleotide is or comprises 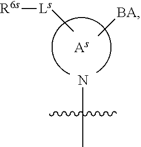
wherein:
R^{6s} is —OH;
Ring A^{s} is an optionally substituted 3-30 membered, monocyclic, bicyclic or polycyclic ring having, in addition to the nitrogen, 0-10 heteroatoms;
each R^{5s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
L^{s} is L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

71. The method of claim 70, where the N is bonded to —C(O)—R^{M}, and/or L^{s} is —C(R^{5s})₂, wherein each R^{5s} is independently R^{s}, and/or wherein L^{s} is optionally substituted —CH₂—, and/or wherein L^{s} is —CH₂—, and/or wherein 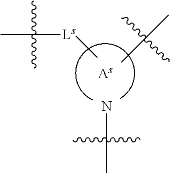
is optionally substituted 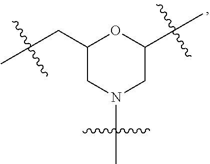 [SMILES: *CC1CN(*)CC(*)O1]
and/or wherein 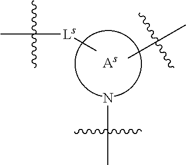
is optionally substituted 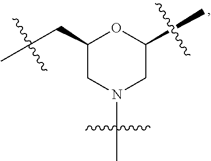
and/or wherein 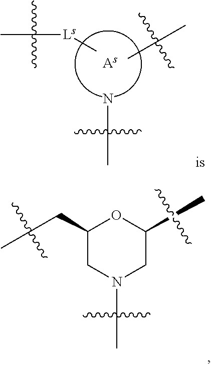
and/or wherein

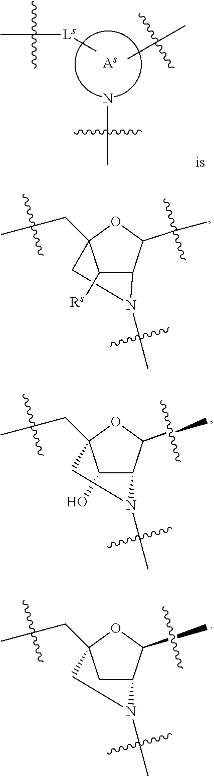

72. The method of any one of claims 60-69, wherein the nucleoside or oligonucleotide is or comprises 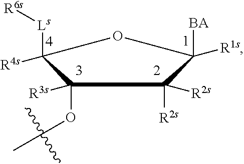
wherein:
R^{6s} is —OH;
each of R^{1s}, R^{2s}, R^{3s}, R^{4s}, and R^{5s} is independently R^{s};
each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
L^{s} is L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

73. The method of claim 72, wherein Cl is bonded to BA, and/or wherein L^{s} is —C(R^{5s})₂, wherein each R^{5s} is independently R^{s}, and/or wherein L^{s} is optionally substituted —CH₂—, and/or wherein L^{s} is —CH₂—, and/or wherein R¹s is —H, and/or wherein R^{3s} is —H, and/or wherein SU is 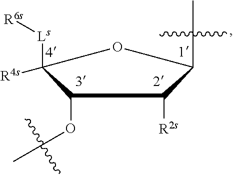
and/or wherein R^{2s} is —H, —OR, wherein R is C₁₋₆ aliphatic, —OMe, or -MOE, and/or R^{4s} is —H, and/or wherein R^{2s} and R^{4s} are taken together to form-L-, and/or wherein R^{2s} and R^{4s} are taken together to form-L-, wherein L is 2′-O—CH₂-4′, wherein the —CH₂— is optionally substituted.

74. The method of any one of claims 60-69, wherein SU is an acyclic sugar or -L^{PS}—SU′-R^{6s}, and/or wherein R^{6s} is —OH protected for oligonucleotide synthesis, and/or wherein L^{PS} is —O—, —NR′—, or a covalent bond, and/or wherein R^{6s} is —O-L-R′, and/or wherein R^{6s} is —OH protected for oligonucleotide synthesis, and/or wherein R^{6s} is DMTrO—.

75. The method of any one of claims 58-74 wherein the second compound is linked to a solid support optionally through a linker, and/or wherein the second compound is a nucleoside or an oligonucleotide linked to a solid support through a linker, and/or the linker comprises one or more —N(R′)—, wherein R′ is not —H, and/or wherein the linker comprises one or more —(CH₂)m—N(R′)—C(O)—(CH₂)n—C(O), wherein R′ is not —H, each of m and n is independently 1-20, and each —CH₂— is independent optionally substituted, and/or wherein the linker comprises one or more —(CH₂)m—N(R′)—C(O)—(CH₂)₂—C(O), wherein R′ is not —H, each of m and n is independently 1-20, and each —CH₂— is independent optionally substituted, and/or the linker comprises one or more —N(R′)—, wherein R′ is optionally substituted C₁₋₆ aliphatic, and/or wherein the linker comprises one or more —N(R′)—, wherein R′ is methyl, and/or wherein the linker comprises no —NH—, and/or wherein the solid support is CPG.

76. The method of any one the preceding claims, wherein the contacting is performed in the presence of a base, optionally wherein the base is DBU, and/or comprising a capping step that comprises a condition under which a —OH group can be capped, and/or comprising a capping step that comprises:
contacting a product of a coupling step with a capping composition comprising a compound having the structure of [R′C(O)]₂, optionally wherein a capping step comprises contacting a product of a coupling step with a capping composition comprising Ac₂O, and/or comprising a deprotection step that comprises a condition under which a protected —OH group can be deprotected, and/or comprising a deprotection step that comprises:
contacting a product of a coupling or a capping step with a deprotection composition comprising an acid, and/or comprising a deprotection step that comprises:
contacting a product of a coupling or a capping step with a deprotection composition comprising an acid, wherein a DMTrO— is converted into —OH.

77. A method, comprising one or more cycles each independently comprising:
a) a coupling step,
b) a capping step, and
c) optionally a deprotection step,
wherein each of the coupling step, capping step, and deprotection step is independently as described in any one of claims 58-76.

78. The method of claim 77, wherein at least one cycle comprise a deprotection step, and/or wherein each one of the one or more cycles independently comprise a deprotection step, and/or wherein the cycle comprise no steps that modify the linkage phosphorus, and/or wherein the coupling step comprises coupling with a compound of any one of claims 49-55.

79. The method of any one of claims 60-78, further comprising one or more cycles each of which independently comprising:
a) a coupling step,
b) optionally a first capping step,
c) a modification step,
d) optionally a second capping step, and
e) optionally a deprotection step,

80. The method of claim 79, wherein the coupling step comprises coupling with a compound of claim 49, and/or comprising a first capping step which comprises an amidation condition, and/or comprising a modification step which modifies a linkage phosphorus form P to P(═O), P═S or P═N—, and/or comprising a second capping step which comprises an esterification condition.

81. A method for preparing a compound of claim 49, comprising contacting a compound of any one of claims 33-37 with a nucleoside.

82. The method of claim 81, wherein the nucleoside is A, T, C or G, optionally protected for oligonucleotide synthesis, and/or wherein the nucleoside is of the structure H—SU—BA or a salt thereof; and/or
wherein SU is 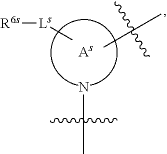
wherein:
R^{6s} is R^{s};
each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
Ring A^{s} is an optionally substituted 3-30 membered, monocyclic, bicyclic or polycyclic ring having, in addition to the nitrogen, 0-10 heteroatoms;
L^{s} is L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy′',
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and/or
wherein the N is bonded to H, and/or L^{s} is —C(R^{5s})₂, wherein each R^{5s} is independently R^{s}, and/or wherein L^{s} is optionally substituted —CH₂—, and/or wherein L^{s} is —CH₂—, and/or wherein 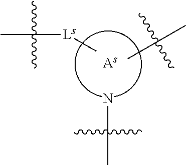
is optionally substituted 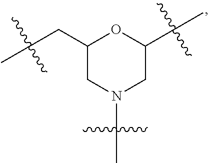 [SMILES: *CC1CN(*)CC(*)O1]
and/or wherein 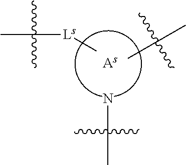
is optionally substituted 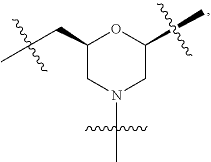
and/or wherein 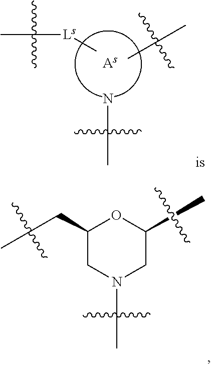
and/or wherein 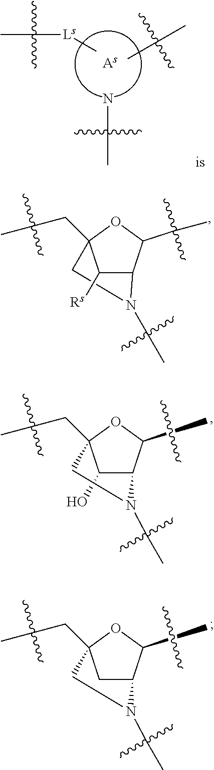
and/or
wherein SU is 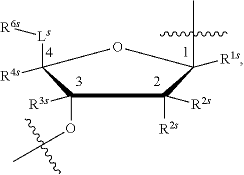
wherein:
each of R^{1s}, R^{2s}, R^{3s}, R^{4s}, R^{5s}, and R^{6s} is independently R^{s};
each R^{s} is independently —H, halogen, —CN, —N₃, —NO, —NO₂, -L-R′, -L-Si(R′)₃, -L-OR′, -L-SR′, -L-N(R′)₂, —O-L-R′, —O-L-Si(R′)₃, —O-L-OR′, —O-L-SR′, or —O-L-N(R′)₂;
L^{s} is L;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and/or
wherein Cl is bonded to BA, and/or wherein L^{s} is —C(R^{5s})₂, wherein each R^{5s} is independently R^{s}, and/or wherein L^{s} is optionally substituted —CH₂—, and/or wherein L^{s} is —CH₂—, and/or wherein R¹s is —H, and/or wherein R^{3s} is —H, and/or wherein SU is 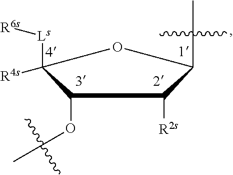
and/or wherein R^{2s} is —H, —OR, wherein R is C₁₋₆ aliphatic, —OMe, or -MOE, and/or R^{4s} is —H, and/or wherein R^{2s} and R^{4s} are taken together to form-L-, and/or wherein R^{2s} and R^{4s} are taken together to form-L-, wherein L is 2′-O—CH₂-4′, wherein the —CH₂— is optionally substituted; and/or
wherein SU is an acyclic sugar or -L^{PS}-SU′- R^{6s}, and/or wherein R^{6s} is —OH protected for oligonucleotide synthesis, and/or wherein L^{PS} is —O—, —NR′—, or a covalent bond, and/or wherein R^{6s} is —O-L-R′, and/or wherein R^{6s} is —OH protected for oligonucleotide synthesis, and/or wherein R^{6s} is DMTrO—.

83. The oligonucleotide, compound, composition or method of any one of the preceding claims, wherein an acyclic sugar has the structure of a′-L^{SA1}-L^{SA2}(-L^{SA3}-)-L^{SA4}-b′, wherein each of L^{SA1}, L^{SA3}, and L^{SA4} is independently optionally substituted bivalent C₁₋₄ aliphatic or C₁₋₄ aliphatic having 1-3 heteroatoms, and L^{SA2} is optionally substituted CH or N, and/or wherein an acyclic sugar has the structure of a′-CH₂—CH(-L^{SA3}-)-CH₂-b′, wherein each of the CH₂ and CH is independently optionally substituted, and -L^{SA3}—- is bonded to a nucleobase, and is —O—CH₂—, wherein the —CH₂— is optionally substituted, and/or wherein an acyclic sugar has the structure of a′-CH₂—CH(—O—CH₂—)—CH₂-b′, wherein each of the CH₂ and CH is independently optionally substituted, and/or wherein an acyclic sugar has the structure of a′—CH₂—CH(—O—CH₂—)—CH(CH₃)-b′, wherein each of the CH₂ and CH is independently optionally substituted, and/or wherein an acyclic sugar has the structure of a′—CH₂—CH(—O—CH(CH₃)—)—CH₂-b′, wherein each of the CH₂ and CH is independently optionally substituted, and/or wherein an acyclic sugar has the structure of a′-CH₂—CH(—O—CH(CH₂OH)—)—CH₂-b′, wherein each of the CH₂ and CH is independently optionally substituted, and/or wherein an acyclic sugar has the structure of a′-CH₂—CH(-L^{SA3}-)-CH₂-b′, wherein each of the CH₂ and CH is independently optionally substituted, and/or wherein an acyclic sugar has the structure of a′-CH₂—CH(O—CH₂—)—CH₂—NHR′-b′, wherein each of the CH₂ and CH is independently optionally substituted, and/or wherein an acyclic sugar has the structure of a′-CH₂—CH(O—CH₂—)—CH₂—N(CH₃)-b′, wherein each of the CH₂ and CH is independently optionally substituted, and/or wherein an acyclic sugar has the structure of a′-CH₂—CH(O—CH(CH₃)—)—CH₂—N(CH₃)-b′, wherein each of the CH₂ and CH is independently optionally substituted, and/or wherein an acyclic sugar has the structure of a′-CH₂—CH(O—CH(CH₂OH)—)—CH₂-b′, wherein each of the CH₂ and CH is independently optionally substituted.

84. The oligonucleotide, compound, composition or method of any one of the preceding claims, wherein each heteroatom is independently boron, oxygen, sulfur, nitrogen, phosphorus, or silicon, and/or wherein each heteroatom is independently oxygen, sulfur, nitrogen, phosphorus, or silicon, and/or wherein each ring heteroatom is independently oxygen, sulfur, or nitrogen.

85. A method for modulating expression, level and or activity of a nucleic acid or a product thereof, comprising contacting the nucleic acid with an oligonucleotide or composition of any one of the any one of the preceding claims, or
a method for modulating expression, level and or activity of a nucleic acid or a product thereof in a system, comprising administering to the system an oligonucleotide or composition of any one of the any one of the preceding claims,
optimally wherein the expression, level and or activity of a nucleic acid or a product thereof is reduced.

86. A method for modulating splicing of a nucleic acid, comprising contacting the nucleic acid with an oligonucleotide or composition of any one of the any one of the preceding claims, or
a method for modulating splicing of a nucleic acid in a system, comprising administering to the system an oligonucleotide or composition of any one of the any one of the preceding claims,
optionally wherein skipping of a target exon is increased, and/or wherein inclusion of a target exon is increased, and/or wherein the nucleic acid is a transcript.

87. The method of any one of claims 85-86, wherein base sequence of the oligonucleotide or oligonucleotides in the composition is complementary or identical to the base sequence of the nucleic acid, and/or wherein a system is an in vitro assay, a cell, a tissue, an organ, an organism, an animal, a subject or a human.

88. The oligonucleotide, composition, compound, or method described in the Specification or of Embodiments 1-743.
